(19) United States
(12) Patent Application Publication
(10) Pub. No.: US 2010/0179765 A1
(43) Pub. Date: Jul. 15, 2010

(54) Novel Methods and Devices for Evaluating Poisons

(76) Inventors: Edwin P. Ching, Woodside, CA (US); Dale E. Johnson, Emeryville, CA (US); Sucha Sudarsanam, Greenbrae, CA (US)

Correspondence Address: WILSON, SONSINI, GOODRICH & ROSATI, 650 PAGE MILL ROAD, PALO ALTO, CA, 94304-1050, US

(21) Appl. No.: 12/572,960

(22) Filed: Oct. 2, 2009

Related U.S. Application Data
(63) Continuation of application No. 11/380,388, filed on Apr. 26, 2006, now abandoned.
(60) Provisional application No. 60/778,133, filed on Mar. 1, 2006.
(60) Provisional application No. 60/675,741, filed on Apr. 27, 2005.

(51) Int. Cl. C12Q 1/68; G06Q 30/00; G06F 19/00

(52) U.S. Cl. 702/19; 435/6; 705/26

(57) ABSTRACT

Methods and devices useful for evaluating poisons or other chemical entities, and for using such methods to forecast unfavorable drug effects.

### PRIORITY CLAIM

This application claims priority to U.S. Provisional Application Ser. No. 60/778,133, filed Mar. 1, 2006 and U.S. Provisional Application Ser. No. 60/675,741, filed Apr. 27, 2005. All the above cited United States provisional applications are expressly incorporated herein by reference in their entirety.

### FIELD OF THE INVENTION

The invention relates to methods and devices for evaluating poisons and other therapeutic entities. Some of the methods and uses are related directly to unfavorable drug effects, and others will be more widely applicable to generic evaluation of pharmacology and therapeutic index.

### BACKGROUND OF THE INVENTION

Developing a new therapeutic drug candidate from initial concept to market sales typically requires from 12 to 15 years of research and development time, and has been estimated to require investing nearly one billion dollars. See, e.g., UBS Warburg Report, Charles River Laboratories, Feb. 28, 2003, pages 7-8; and www.fda.gov. A significant portion of these expenditures occurs during preclinical animal testing, and even more is spent on human clinical safety and efficacy testing. Pharmacology or toxicology problems which remain undetected until later stages of drug development are extremely problematic, both in terms of dollar expenditures and lost time. And the situation is even worse if toxicity problems remain undetected until after market introduction. Thus, early and accurate assessment of safety and efficacy of candidate therapeutic entities, along with proper administration and treatment methods, is indispensable to efficient development of new therapeutic entities. Pharmacology is a science directed to the study of the action of substances, typically chemicals and other entities, on biological systems. This encompasses both pharmacodynamics and pharmacokinetics. See, e.g., Berkow, et al. The Merck Manual Merck and Co.; Hardman, et al (eds. 2001) Goodman and Gilman's: The Pharmacological Basis of Therapeutics (10th Ed.) McGraw-Hill, ISBN: 0071354697; and other academic and professional school textbooks used in teaching pharmacology. The US Food and Drug Administration (FDA) is concerned with virtually all aspects of use of substances in therapeutic or diagnostic contexts. See, e.g., www.fda.gov.

A closely related area of relevance is toxicology, which addresses features and properties of substances which may have specific defined effects on the systems, typically leading to negative or undesirable effects. See, e.g., Klaassen, et al. (eds. 2001) Casarett and Doull's Toxicology: The Basic Science of Poisons (6th ed.) McGraw-Hill, ISBN: 0071347216; and Hayes (ed. 2001) Principles and Methods of Toxicology (4th Ed.) CRC Press, ISBN: 1560328142; and other academic and professional school textbooks used in teaching toxicology.

However, elucidating the pharmacology and toxicology of a substance, e.g., a therapeutic entity or potential new treatment, requires a significant amount of study to determine the optimal means and methods for use in a defined biological context. This is both a costly and laborious process, which requires enormous investment in financial and other resources. This fact is recognized by the US FDA and other national drug regulatory agencies. It has been stated that commercially available tests for safety monitoring (biomarkers) are urgently needed, and such remains the least developed area of pharmacogenomic monitoring and individualized medical treatment.

Citation of documents above and hereafter is not intended as an admission that any is pertinent prior art. All statements as to the date or representation as to the contents of documents is based on the information available to the applicant and does not constitute any admission as to the correctness of the dates or contents of the documents.

### SUMMARY OF THE INVENTION

The present invention is directed to accelerating the speed of development and reducing the resource investment necessary to determine these features for directing use of such substances or treatments to appropriate biological contexts.

The present invention provides lists of biomarkers for analysis, either directly or indirectly, which affect the toxicity pathways. These may be evaluated at many levels, including genetic, genotyping, evaluation of combination pairing of diploid alleles or haplotypes, RNA expression, protein expression, functional activity, post-translational analysis or evaluation, etc. Thus, the biomarkers refer to the corresponding genetic information, RNA, protein, or other structural embodiments thereof. And the means to use these biomarkers, e.g., to evaluate status of toxicity pathways, to evaluate individual risk or susceptibility to various toxic pathways from exposure or therapeutic intervention, to generate test systems for drug development, are all provided by identifying critical and significant contributors to the pathway progression.

The invention further provides methods for detecting the state of a toxicity pathway in a primate, said method comprising evaluating the form or function of a discriminatory biomarker selected from: (a) Table 4, subset 1; (b) Table 4, subset 2; (c) Table 4, subset 3; (d) Table 3A or 6A, subsets 2 or 3; Table 3A or 6A, subset 1; Table 2A or 5A, subsets 2 or 3; and (e) Table 2A or 5A, subset 1. Specific datasets also provide various markers, individually or in various combinations. Various pluralities or combinations of those markers are important in liver or other toxicity pathways. In various embodiments: the toxicity pathway is affected in response to a therapeutic treatment, including administration of a drug or combination of therapies; the primate is a chimpanzee; the form of evaluating is determination of genetic presence of a specific allelic form or specific combination of diploid alleles of said discriminatory biomarker; the form of evaluating is expression at a nucleic acid or protein level, including allelic diploid combinations of said discriminatory biomarker; the form of evaluating is a protein evaluation, including an immunoassay, modification, quantitation, mass spectroscopy, NMR, imaging, or characteristic temporal pattern determination; the form of evaluation is determination of functional activity of said discriminatory biomarker, including a detectable substrate or product of an enzymatic activity affected by said biomarker; the form of evaluation is expression or functional localization of said discriminatory biomarker in said primate, including imaging or localization; the evaluating is from a blood, hair, skin, saliva, or accessible body fluid sample or part; the evaluation includes a plurality of discriminatory biomarkers in said subsets, including biomarkers from a plurality of different subsets, including from Tables 2A/B, 3A/B, 4, 5A/B, or 6A/B; or the evaluation is a non-invasive method, including an imaging agent or detectable or labeled compound.

Based upon these identified biomarkers, the invention provides label, diagnostic reagent, or diagnostic means directed to the identified discriminatory biomarker(s); and various kits comprising such and instructions or devices for using such and/or interpreting the results there from.

In preferred embodiments, the kit: (i) evaluates a multiplicity of biomarkers from Table 4; (ii) is designed to evaluate or distinguish between a plurality of defined liver toxicity pathways; (iii) is designed to further evaluate other toxicity pathways other than in liver; or (iv) is designed to evaluate a status of a toxicity pathway induced by a therapy or drug; or the diagnostic reagent or means: (i) evaluates presence or absence of specific alleles corresponding to said discriminatory biomarker; (ii) evaluates presence or absence of specific diploid combinations of alleles or haplotypes corresponding to said discriminatory biomarker; (iii) evaluates a plurality of said discriminatory biomarkers; (iv) evaluates said discriminatory biomarker over multiple time points; or (v) evaluates at least one other marker or feature.

The invention also provides test systems for chemical or biologic compounds, to screen or evaluate the impact on toxicity or other pathways affected by said compounds. In preferred embodiments, the test system: (a) incorporates a plurality of the identified discriminatory biomarkers; (b) incorporates a plurality of different features of said discriminatory biomarkers; (c) is designed to also evaluate status of non-liver toxicity pathways; or (d) evaluates various features of biomarkers selected from the identified discriminatory biomarkers.

A computer system is further provided which: (a) includes a file which provides listings of discriminatory biomarkers including at least one identified biomarker linked to status of toxicity pathways; (b) is capable of providing output of specific features of identified biomarkers which are indicative of status of toxicity pathways in particular patient subclasses; or (c) includes a file which links appropriate features of appropriate identified biomarkers, in addition to appropriate features of biomarkers for different pathways of toxicity in muscle, neurological, or bone tissue.

In other embodiments, the present invention provides methods of correlating the state of a toxicity pathway to a combination of diploid haplotypes present in a biological system. In certain embodiments, the toxicity pathway is: expressed significantly in liver, muscle, neurological, or bone marrow; expressed primarily in the GI tract, kidney, or skin; induced by a therapeutic treatment; or is induced by administration of one or a combination of drugs; or the combination of diploid haplotypes: represent at least 60% of the allelic combinations found in the US, Western Europe, or Japanese national populations; represent at least 15 different genes; represent at least 7 non-contiguous haplotype blocks; represent at least 4 different non-Y chromosomes; span at least 100 centimorgans; include a plurality which are derived from a vertebrate; are evaluated by characterization of protein features, e.g., by ELISA; or include some haplotypes from a primate; or the biological system is: a soluble test system; a cell line; an organ system; or an animal; or the correlating is: performed on a computer, which collates data to generate a file of particular identified combinations of alleles which exhibit defined categories of risk from said status of said toxicity pathway; or used to develop a set of combination diploid haplotypes which are correlated and validated to be incorporated into a diagnostic product, including one useful to predict toxicity pathway status in a subject.

The invention further provides methods of identifying additional relevant genes as candidate test targets for toxicity pathway evaluation, by taking a first list of candidate targets and identifying a second list of additional candidate targets: (1) which in an interaction database have been reported to interact physically with said targets of list 1, including a physical interaction or 2-hybrid physical interaction; (2) which have been commonly referred to in a reference with a target of list 1, said reference being in the abstract of a paper contained in a literature database; (3) whose gene expression profiles match the expression profiles of those members of list 1 in similar tissues; (4) which have been co-localized in expression analyses in similar tissues; or (5) which are closely located physically on a chromosome. In various embodiments, the toxicity pathway is: expressed significantly in liver, muscle, neurological, or bone marrow; expressed primarily in the GI tract, kidney, or skin; is induced by a therapeutic treatment; or is induced by administration of one or a combination of drugs; or the first subset of candidate targets are derived from some screening methodology, including SNP analysis, gene expression profiling, post-translational modification analysis, and mass spectroscopy; or the second list: contains fewer than three times as many candidates as list 1; contains at least 20 candidate targets; contains at least 20% metabolic enzymes or transporters; is screened to validate members thereof which can classify status of said toxicity pathway into categories of risk; or the interaction database: includes data from the NCBI or PubMed databases; comprises at least 10,000 reports of physical interactions; uses manual collation, gene symbol designation, and/or word term matching; or the literature database: comprises at least 200,000 documents; contains completely abstracts of at least 100 journals since 1990; contains completely abstracts of at least 1000 journals since 1970; contains at least 20 thousand document abstracts; contains at least 500 thousand document abstracts; is available from Ingenuity or GeneGo; includes the NCBI and/or PubMed literature databases; or the gene expression profiles: match in one or more of liver, muscle, brain, bone, GI tract, kidney, skin, or oral mucosa; are in the same organ and physiology as those in list 1; are in a primate; or the additional candidates exhibit at least 2 of the criteria for inclusion into said second list; or the identifying is: performed on a computer which maintains the list 2 of candidate genes, which are subjected to validation to develop a diagnostic product there from; or followed by validation of relevance of candidates as classifier biomarkers to toxicological status, which may lead to efforts to develop a diagnostic product.

Other methods are provided herein of correlating status of a toxicity pathway to temporal patterns of features of classifier biomarkers determined at multiple time points, e.g., within a single individual, wherein said feature is selected from: (1) RNA expression of selected genes; (2) protein expression of selected genes; (3) post translational features of selected genes; (4) metabolic conversions of reactants or products of selected genes; (5) cellular, organ, or tissue localization of a biological product or tracer (including nucleic acid, protein, carbohydrate, phosphorylation, label, or toxin); or (6) features of acute liver metabolic enzymes or transporters. Preferred embodiments include, e.g., those where: the toxicity pathway is: expressed significantly in liver, muscle, neurological, or bone marrow; expressed primarily in the GI tract, kidney, or skin; is induced by a therapeutic treatment; or is induced by administration of one or a combination of drugs; or where the temporal pattern is an increase, decrease, stable then change, increase then decrease, or decrease then increase; or the classifier biomarker is evaluated in a whole organism, including a primate; or the time points span: hours to weeks to months; from before to after one or more toxicity symptom is manifested; or the classifier biomarker is assayed by an imaging agent, a test reagent, or detectable reactant or product; or the correlating is: performed on a computer, which collates data to generate a file of particular identified temporal patterns of features which define categories of risk from said status of said toxicity pathway; or used to develop a set of identified temporal patterns of features which are correlated and validated to be incorporated into a diagnostic product, including one useful to predict toxicity pathway status in a subject.

Yet other methods are provided correlating status of a toxicity pathway to classifier biomarkers, wherein: (1) said markers are monitored in a genetically homogeneous primate population with substantial medical records allowing generation or testing of correlation of said status of toxicity pathway with said biomarkers in said population; or (2) a sufficiently large population of primates with access to: (i) primate biological samples; or (ii) sufficient diagnostic data within the record, such allowing selection of a subset of said population with sufficient numbers to evaluate from said subset correlation of non-therapy related toxicity to classifier biomarkers. Various particular embodiments include where the toxicity pathway is: expressed significantly in liver, muscle, neurological, or bone marrow; expressed primarily in the GI tract, kidney, or skin; not induced by a therapeutic treatment; or induced by administration of a combination of drugs; or the classifier biomarkers: include a plurality of both metabolic enzymes and transporters; or number at least 10 different classifier biomarkers; or the genetically homogeneous population: has accessible medical records and informed consent for at least 30 thousand individuals; is located in the US; is from Finland, Iceland, Sardinia, or Estonia; has essentially full medical records for individuals for at least 5 years previous to testing of biomarkers; has a LD of less than 0.80 on a median intermarker distance of 4.5 KB; or has highly conserved mitochondrial DNA sequence; or the samples are archived or banked; or the subset has phenotypic homogeneity by selection criteria; or the correlating is: performed on a computer, which collates data to generate a file of particular genotypes or other features which define categories of risk from said status of said toxicity pathway; or used to develop a set of genotypes or features which are correlated and validated to be incorporated into a diagnostic product, including one useful to predict toxicity pathway status in a subject.

The invention further provides combinations of the methods, e.g., studying biology of a mammal, comprising combining a method of correlation analysis between phenotype and a diploid haplotype with extending a list of functional candidate entities from list 1 to list 2 by system biology linkage, which may include linkage by physical interaction and/or literature connection by common reference in a published abstract. Exemplary embodiments include wherein (i) a list 1 is extended to a list 2, and said phenotype is further correlated with diploid haplotype combinations corresponding to at least one functional candidate of list 2; (ii) a diploid analysis is performed, and the phenotype is further correlated with another feature of a functional candidate of list 2 resulting from extending of a list 1 of candidates evaluated in said diploid analysis; or (iii) a diploid analysis is performed, and the phenotype is further correlated with another feature of a functional candidate of list 2 resulting from correlation to a list 1 candidate resulting from analysis of a different parameter.

Additional methods are provided, e.g., which combine methods of correlation analysis between phenotype and a combination of diploid haplotypes with evaluating multiple time point features, which may include haploid or combination diploid analysis. Certain embodiments include, e.g., wherein correlation between said biology in said mammal is with: (i) at least one diploid haplotype combination and at least one multiple time point feature; or (ii) a plurality of diploid haplotype combinations and multiple time point features.

The invention further provides combining methods of correlation analysis between phenotype and a plurality of non-adjacent haplotypes with use of a “homogeneous” primate population, which may include genetically homogeneous or phenotypically selected “subclasses” from a larger collection by medical record or other selection criteria. In some embodiments, the population is a genetically homogeneous population; or the biology is not a response to treatment.

Other methods extend a list of functional candidates from list 1 to list 2 by system biology linkage, which may include linkage by physical interaction and/or literature connection by common reference in a published abstract, with evaluating multiple time point features, which may include haploid or combination diploid tracking. Preferred embodiments include where: (i) the correlation between said biology in said mammal is with at least one parameter of a list 2 candidate and one multiple time point feature; (ii) the correlation is with a multiple time point feature of a list 2 candidate; or (iii) the correlation is with a feature of a list 2 candidate resulting from a multiple time point analysis.

Yet another embodiment of the invention results from combining methods to extend a list of functional candidates from list 1 to list 2 by system biology linkage, which may include linkage by physical interaction and/or literature connection by common reference in a published abstract, with methods which use a “homogeneous” primate population, which may include genetically homogeneous or phenotypically selected “subclasses” from a larger collection by medical record or other selection criteria. In preferred forms, this may include where: (i) a correlation of said biology in said mammal to a list 1 candidate leads to said list 2 candidate, which is tested for validation in said primate population; or (ii) a hypothesis generated from said population directed to a list 1 candidate is tested by evaluating a list 2 candidate.

Further methods result from combining methods to evaluate multiple time point features, which may include haploid or combination diploid analysis, with use of a “homogeneous” primate population, which may include genetically homogeneous or phenotypically selected “subclasses” from a larger collection by medical record or other selection criteria. Among such embodiments is where the biology is tested in said homogeneous primate population for correlation with multiple time point features.

Also provided are methods using analysis of genetic makeup of a target individual animal to predict therapeutic outcome from administration of a compound or treatment to the target individual, the method involving: establishing correlation of therapeutic outcomes to various combinations of haplotypes or alleles possessed by various individual animals; determining the combination of haplotypes or alleles possessed by the target individual; and applying the correlation from the combination of haplotypes or alleles to predict the therapeutic outcomes. Alternatively, the methods comprise determining the combination of alleles possessed by the target individual (and previously established as correlated with the therapeutic outcome) and; and applying the correlation from the combination of alleles to predict the therapeutic outcomes.

In certain embodiments of the methods, the analysis of the genetic makeup is qualitative or quantitative determination of common haplotypes or alleles across a population of which the target individual is a member, including analysis of haplotype or allele dosage; the analysis is by nucleic acid (DNA, RNA) sequence or polymorphism analysis, (DNA; RNA) hybridization, protein analysis, or enzyme activity analysis; the genetic makeup includes: duplication or multiple copies of an allele or haplotype, chromosome duplication, amplification of a genetic locus, or multiple related alleles of at least 90% amino acid sequence identity over a length of at least 35 amino acids; the target individual is: a primate, rodent, or canine; a companion, work, or show animal; a quadruped, biped, or aquatic animal; a vertebrate, including one with an exoskeleton; or heterozygous or homozygous with respect to the haplotype or allele; or the therapeutic outcome is: a drug adverse event; no drug adverse event; drug efficacy; or no drug efficacy. Yet other methods include those where the administration is: one or more purified chemical entity or compound; topical, oral, parenterally, inhaled, administered to the eye, an implant, or other means; or repeated; or the correlation: is with a coefficient greater than 0.6; has been established with a statistical reliability measure; has been established by testing of a drug adverse event population of greater than 100 adverse events; is combined with another feature from a medical record of the target individual or with another diagnostic result; or is made in a homogeneous founder population of at least 20K individuals; or the allele is in a: cytochrome P450 locus; transporter/pump locus; or “drug metabolizing enzyme” locus. Further methods include those comprising communicating to a recipient a result of the method, wherein: the communication is: written, oral, coded, digital, analog, or passes through US legal jurisdiction; or where the recipient is: within US legal jurisdiction; a medical patient or veterinary owner; a health care professional, medical or veterinary; a regulatory agency or drug development organization; or a health care insurer or auditor.

In another embodiment, the invention provides diagnostic devices comprising means to determine a substantially full complement of haplotypes or alleles of a biomarker possessed by a target diploid individual, the means providing for identifying what haplotypes or alleles are present in the target individual, and evaluating biological function of the product of those haplotypes or alleles. Often, the devices will be ones wherein: the means: simultaneously determine both what haplotypes are present or absent, and what biological function corresponds to the haplotypes; determine the complete protein sequence encoded by each haplotype; are automated and provide a readout result within about three hours; or include dynamic features and/or multiple time points of evaluating; the complement of haplotypes includes: a heterozygous pair of haplotypes; a gene dosage variation different from a chromosomal pair, including a chromosome duplication resulting in triploidy of the chromosome; a plurality of closely related sequences which exhibit both high sequence identity and overlapping biological function (multiple homologs, e.g., where complement of related enzymes affect selectivity/specificity/kinetics of reaction, or transporters); alleles of enzymatic turnover numbers which differ by at least 30%; or surrogate markers which are accepted as diagnostic for a defined phenotype. Other embodiments may be devices where: the biomarker: comprises a plurality of a cytochrome, enzyme, transporter, and/or structural protein; or is represented by at least 5 different alleles or non-contiguous haplotypes found in a population including the individual; or the diploid individual is: a mammal, including a primate, rodent, feline, or canine; a companion, work, or show animal; or an experimental research animal, including a nematode, water flea, insect, or invertebrate; or the evaluating biological function is: by proteomic or metabolomic analysis; or capable of distinguishing different types of pharmacological dose response curves, including an increasing or decreasing, U shaped, bell shaped, or hormetic situation.

Methods using such devices are provided, e.g., methods comprising predicting outcome from a defined treatment of a target individual by evaluating the complement of alleles possessed by the individual using the described device; or where: the outcome is therapeutic efficacy, therapeutic safety, or risk of an adverse reaction; or results of the evaluating are communicated to a recipient wherein: the communication is written, oral, coded, digital, analog, or passes through US legal jurisdiction; or the recipient is: within US legal jurisdiction; a medical patient or veterinary owner; a health care professional, medical or veterinary; a regulatory agency or drug development organization; or a health care insurer or auditor.

Another alternative embodiment of the invention provides methods of identifying biomarkers useful for predicting response of an individual to therapeutic treatment, comprising: collecting a homogeneous population of individuals having received the treatment with a recorded result from the treatment; evaluating genetic markers in a plurality of the individuals in the population to identify biomarkers which correlate with specific recorded result from the treatment; and correlating the genetic markers with the specific recorded results to identify biomarkers which are predictive of the result. Among various methods encompassed include, e.g., those where: the identifying: allows development of a registered diagnostic test or device which evaluates the biomarker, e.g., to predict an adverse drug response; is communicated by written, oral, coded, digital, analog, or means passing through US legal jurisdiction; or is communicated to a recipient who is within US legal jurisdiction; a medical patient or veterinary owner; a health care professional, medical or veterinary; a regulatory agency or drug development organization; or a health care insurer or auditor; or biomarker includes: a dynamic or temporal component in evaluation; multiple endpoint, concentration, temperature, or other analyses; a genetic analysis; or a proteomic and/or metabolomic analysis; or the prediction has an accuracy of at least 95% over a defined population; or the response is a pharmacological or toxicological response.

Other methods encompassed include some, e.g., where the individual is: a mammal, including a primate, equine, bovine, porcine, canine, feline, rodent, or quadruped; a companion, work, or show animal; an experimental research animal, including a nematode, water flea, insect, or invertebrate; or a plant, fungus, protozoa, or prokaryote; or the treatment is administering one or more therapeutic compounds in a predetermined methodology; or the population: comprises at least 2 million individual primates; has a homogeneity exhibiting fewer than about 300K SNPs of frequency occurrences of at least 1% within the population; has medical records accessibility for at least 30% of the population going back at least 3-5 years; and/or has an adverse drug reaction reporting system.

The invention further provides such methods wherein the genetic markers allow prediction of other biomarkers from pathways correlated to the result, and the other biomarkers from the pathways may be tested by perturbations to optimize or identify what perturbations affect correlation of the biomarkers to the result, thereby identifying high correlation biomarkers for the result. This shall include methods wherein: the perturbations: are in a gene sequence or quantity (regulation); protein sequence, modification, or quantity; substrates or analogs thereof (including inhibitors or regulatory subunits); metabolic intermediates; time of endpoint or analyses; temperature; and/or isotopic variants; are achieved by any of gene expression modifiers (including knockout or transformants), gene suppression (e.g., using RNAi or anti-sense), use of dominant negative forms or suppressors, and activating mutants; are achieved by chemical perturbations, e.g., by varying concentrations of small molecule inhibitors, co-factors (natural or otherwise), or activators; and/or are evaluated as a function of time; and/or the high correlation biomarkers can be: incorporated into an experimental system which can be used to model effect of a therapeutic treatment back to a target individual or subsystem thereof; monitored in an individual to anticipate the timing, severity, or type of a phenotype, e.g., as a pool of surrogate markers; or diagnosed in an individual and predict efficacy or response to a therapeutic treatment, including an adverse drug response.

Further methods are provided, e.g., wherein: the experimental system comprises: a transgenic, transformed, or genetically modified cell; an identified selected genetic, developmental, or physiological variant cell; an in vitro genetic model for a disease; an organism, including a rodent, possessing features characterizing a disease or model; a cell comprising a human gene; a candidate therapeutic entity for treatment of a medical condition; a mammalian stem cell, including a cell derived from a primate; or at least one of the steps occurs outside the United States.

In a drug development context, the method may comprise use of the experimental system to evaluate or prioritize development candidates for pharmacology or toxicology, e.g., in preclinical evaluation.

The invention further provides methods using a combination of cells or systems comprising genetic or physiological variants exhibiting specific high correlation biomarkers for a phenotype, wherein the combination of cell lines is monitored to evaluate therapeutic response to a therapeutic treatment. This will include methods where: the combination of cells or systems: comprise one or more human gene, chromosome, or cell; evaluate effect of different expression levels of one or more haplotype, gene, or phenotype; make use of one or more microfluidic chips, e.g., allowing a series of chips to represent various individuals in a population; provide a model for disease, including an in vitro or in vivo model; provide a surrogate marker for a human or animal phenotype, including toxicity; or are in an intact organism; or where the monitoring evaluates multiple endpoints, a concentration/response, metabolic turnover (including substrate and/or product), a plurality of different assays, and/or multiple genetic variants; or where the phenotype: is in a primate or invertebrate; or allows prediction of therapeutic index of a therapeutic entity in a defined system or animal; or where the cells or systems represent a scope of variation of individuals across a population of the individuals; or where the therapeutic treatment is testing or screening various candidate therapeutic entities, including prioritization of candidates for product development; or where results of evaluation or conclusion resulting is communicated to a recipient, wherein: the communication: is written, oral, coded. digital, or analog, or passes through US jurisdiction; or the recipient is: within US legal jurisdiction; a medical patient or veterinary owner; a health care professional, medical or veterinary; a regulatory agency or drug development organization; or a health care insurer or auditor.

### DETAILED DESCRIPTION OF THE INVENTION

### Outline

### I. Introduction

### II. Gene And Haplotype Pairings Correlated to Phenotype

### III. Correlated Genes to Pathway Hypotheses

### IV. Hypotheses to Optimized Signatures

### V. Optimized Signature Relevance to Humans

### VI. Optimized Biomarkers/Signatures; to Cell Lines, Experimental Models

VII. in vitro and in vivo Disease Models

### VIII. Output Products

### IX Computer Systems

### X. Business Methods

### I. Introduction

As described above, the need for commercially available tests for safety monitoring (biomarkers) are urgently needed. The US FDA and other groups have formed brainstorming discussion groups such as the industry sponsored Pharmacogenetics Working Group to chart out new strategies and development in this area. Similar concerns exist in foreign drug regulatory agencies. Activity in the pharmacogenomics area is great, including release of the Guidance for Industry Pharmacogenomic Data Submissions (November 2003); a draft Preliminary Concept Paper “Drug-Diagnostic Co-Development Concept Paper” (April 2005); and other papers and guidelines scheduled for release from the FDA in the near future. Coupled with a recent focus on toxicology issues of marketed drugs (Vioxx, Bextra, and others), along with slow and costly development for new drugs, pharmacogenomics has become a higher profile endeavor.

The science of drug development is quite complex. It includes, among others, various areas of study of medical sciences and particularly therapeutic effects, including the sciences of pharmacology and toxicology, along with medicinal chemistry, diagnostic principles, statistics, and validation procedures. Pharmacology is directed to the study of the properties and reactions of drugs especially with relation to their therapeutic values. Various aspects of pharmacology include formulation, adsorption, distribution, metabolism, excretion, and such. See, e.g., Evans (2004) A Handbook of Bioanalysis and Drug Metabolism CRC Press, ISBN: 0415275199; Golan, et al. (2004) Principles of Pharmacology: The Pathophysiologic Basis of Drug Therapy Lippincott Williams and Wilkins, ISBN: 0781746787; Minneman (2004) Brody's Human Pharmacology: Molecular To Clinical (4th ed.) Mosby-Year Book; ISBN: 0323032869; van de Waterbeemd, et al. (2003) Drug Bioavailability: Estimation of Solubility, Permeability, Absorption and Bioavailability (Methods and Principles in Medicinal Chemistry) Wiley-VCH, ISBN: 352730438X; Avdeef (2003) Absorption and Drug Development: Solubility, Permeability, and Charge State Wiley-Interscience, ISBN: 0471423653; Allen (2002) The Art, Science, and Technology of Pharmaceutical Compounding (2d ed.), APhA Pub., ISBN: 1582120358; Amiji and Sandmann (2002) Applied Physical Pharmacy McGraw-Hill Med., ISBN: 0071350764; Smith, et al. (2000) Pharmacokinetics and Metabolism in Drug Design (Methods and Principles in Medicinal Chemistry) Wiley-VCH, ISBN: 3527301976; Testa and Mayer (2003) Hydrolysis in Drug and Prodrug Metabolism: Chemistry, Biochemistry, and Enzymology Wiley-VCH, ISBN: 390639025X; Ansel and Stoklosa (2001) Pharmaceutical Calculations (11th ed.) Lippincott Williams and Wilkins, ISBN: 0781731720; Daniels, et al. (eds. 2001) Principles of Clinical Pharmacology Academic Press; ISBN: 0120660601; Gibson and Skett (2001) Introduction to Drug Metabolism (3d ed.) Nelson Thornes, ISBN: 0748760113; Ansel, et al. (1999) Pharmaceutical Dosage Forms and Drug Delivery Systems (7th ed.) Lippincott Williams and Wilkins, ISBN: 0683305727; Cannon (1999) Pharmacology for Chemists (ACS Professional Reference Book) Amer. Chem. Soc., ISBN: 0841235244; Woolf (1999) Handbook of Drug Metabolism Marcel Dekker, ISBN: 0824702298; and Feldman, et al (1996) Principles of Neuropsychopharmacology Sinauer Associates, ISBN: 0878931759. See also Frank and Hargreaves (2003) “Clinical biomarkers in drug discovery and development” Nature Drug Discovery 2:566-580.

Toxicology textbooks include, besides those used in standard academic or professional school courses, Moffat, et al. (2004) Clarke's Analysis of Drugs and Poisons (3d ed.) Pharmaceutical Press, ISBN: 0853694737; Hodgson (ed. 2004) A Textbook of Modern Toxicology (3d ed.) Wiley-Interscience, ISBN: 047126508X; Burczynski (2003) An Introduction to Toxicogenomics CRC Press, ISBN: 0849313341; Boelsterli (2002) Mechanistic Toxicology: The Molecular Basis of How Chemicals Disrupt Biological Targets CRC Press, ISBN: 0415284597; Rossoff (2001) Encyclopedia of Clinical Toxicology: A Comprehensive Guide Taylor and Francis Group, ISBN: 1842141015; Gad, et al. (2001) Regulatory Toxicology (2d ed.) Taylor and Francis STM, ISBN: 0415239192; Hodgson and Smart (2001) Introduction to Biochemical Toxicology (3d ed.) Wiley-Interscience, ISBN: 0471333344; Lewis (2001) Guide to Cytochromes P450: Structure and Function CRC Press, ISBN: 0748408975; Ford, et al. (eds. 2000) Clinical Toxicology Saunders, ISBN: 0721654851; Wexler, et al. (2000) Information Resources in Toxicology (3d ed.) Academic Press, ISBN: 0127447709; Wexler, et al (1998) Encyclopedia of Toxicology (3 Vol.) Acad. Pr., ISBN: 012227220X; Puga and Wallace (1998) Molecular Biology of the Toxic Response CRC Press, ISBN: 1560325925; and Sipes, et al. (eds. 1997) Comprehensive Toxicology (13 vols.) Elsevier, ISBN: 0080423019 (CD-Rom ed. ISBN: 008042306X).

Medicinal chemistry is a critical function in drug development, and is described generally, e.g., in Dingermann, et al. (2004) Molecular Biology in Medicinal Chemistry (Methods and Principles in Medicinal Chemistry) Wiley, ISBN: 3527304312; Silverman (2004) The Organic Chemistry of Drug Design and Drug Action (2d ed.) Acad. Pr., ISBN: 0126437327; Abraham (ed. 2003) Burger's Medicinal Chemistry and Drug Discovery (6 Vols. on Drug Discovery; Drug Discovery and Drug Development; Autocoids, Diagnostics, and Drugs from New Biology; Cardiovascular Agents and Endocrines; Chemotherapeutic Agents; and Nervous System Agents) Wiley-Interscience, ISBN: 0471370320; Lemke (2003) Review of Organic Functional Groups: Introduction to Medicinal Organic Chemistry (4th ed. with CD), Lippincott Williams and Wilkins, ISBN: 0781743818; Wermuth (ed. 2003) The Practice of Medicinal Chemistry (2d ed.) Academic Press; ISBN: 0127444815; Williams, et al. (2002) Foye's Principles of Medicinal Chemistry (5th ed.) Lippincott Williams and Wilkins, ISBN: 0683307371; Patrick (2001) An Introduction to Medicinal Chemistry (2d ed.) Oxford Univ. Pr., ISBN: 0198505337; Smith, et al (2000) Pharmacokinetics and Metabolism in Drug Design (Methods and Principles in Medicinal Chemistry) Wiley-VCH, ISBN: 3527301976; Dickson (1998) Medicinal Chemistry Laboratory Manual: Investigations in Biological and Pharmaceutical Chemistry CRC Press, ISBN: 0849318882; and King (1994) Medicinal Chemistry: Principles and Practice Springer-Verlag, ISBN: 0851864945.

Systems biology analyses and techniques are described, e.g., in Klipp, et al. (2005) Systems Biology in Practice: Concepts, Implementation and Application Wiley, ISBN: 3527310789; Kitano (ed. 2001) Foundations of Systems Biology MIT Press, ISBN: 0262112663; Bower and Bolouri (eds. 2001) Computational Modeling of Genetic and Biochemical Networks (Computational Molecular Biology) MIT Press, ISBN: 0262024810; Voit (2000) Computational Analysis of Biochemical Systems: A Practical Guide for Biochemists and Molecular Biologists (with CD-ROM) Cambridge Univ. Pr., ISBN: 0521785790; and other materials used in leading academic or professional school departments teaching courses in this area. Fundamental principles may include, e.g., coregulation suggesting functional relationship, and others which are based upon information theory and mathematics of complex systems, of which biology is one of the most complex. See, e.g., Abraham, et al. (2004) “High content screening applied to large-scale cell biology” Trends Biotechnol. 22: doi:10.1016/j.tibtech.2003.10.012; directed to cell biology, but systems biology indicates that cell biology affects organ physiology and system response.

Other references relevant to the subject matter of the present invention include, e.g., Cavalli-Sforza and Bodmer (1971) The Genetics of Human Populations Freeman, San Francisco; Babine, et al. (2004) Protein Crystallography in Drug Discovery (Methods and Principles in Medicinal Chemistry) Wiley, ISBN: 3527306781; Kumar, et al. (2004) Robbins and Cotran: Pathologic Basis of Disease (7th ed. with CD) Saunders Co., ISBN: 0721601871; Kubinyi, et al. (2004) Chemogenomics in Drug Discovey: A Medicinal Chemistry Perspective (Methods and Principles in Medicinal Chemistry) Wiley, ISBN: 352730987X; Block, et al. (2004) Wilson and Gisvold's Textbook of Organic Medicinal and Pharmaceutical Chemistry (11th ed.) Lippincott Williams and Wilkins, ISBN: 0781734819; Böhm, et al. (2003) Protein-Ligand Interactions: From Molecular Recognition to Drug Design (Methods and Principles in Medicinal Chemistry) Wiley, ISBN: 3527305211; Seydel, et al. (2002) Drug-Membrane Interactions: Analysis, Drug Distribution, Modeling (Methods and Principles in Medicinal Chemistry, Volume 15) Wiley-VCH, ISBN: 3527304274; Smith, et al (2000) Pharmacokinetics and Metabolism in Drug Design (Methods and Principles in Medicinal Chemistry) Wiley-VCH, ISBN: 3527301976; Wolff (ed. 1997) Therapeutic Agents, Volume 4, Burger's Medicinal Chemistry and Drug Discovery (5th ed.) Wiley-Interscience, ISBN: 0471575593; and Kennewell (1991) Comprehensive Medicinal Chemistry: General Principals Pergamon Pr., ISBN: 0080370578. Additional references of general medical relevance include, e.g., Berkow (ed.) The Merck Manual of Diagnosis and Therapy Merck and Co., Rahway, N.J.; Thorn, et al. Harrison's Principles of Internal Medicine McGraw-Hill, N.Y.; and Weatherall, et al. (eds.) Oxford Textbook of Medicine Oxford Univ. Press, Oxford.

### II. Gene and Haplotype Pairings Correlated to Phenotype

### A. Phenotypic Features

Phenotypes are diverse, and relate e.g., to physiological, metabolic, behavioral, health status, disease state, development, and other functional or structural characteristics of a system. Features may be diverse as size, weight, color, function, histology evaluation, or other distinctive features of the system or parts thereof. The evaluation may be of the entire system together, or of parts thereof, e.g., function of particular organ subsystems or metabolic pathways. Among the phenotypes of interest herein include response to therapy, including efficacy, or toxicological response, including the standard adsorption, distribution, metabolism, excretion, and negative response to an administered drug or therapy. Negative responses are often characterized as adverse drug responses (ADR). The pharmacological features often evaluate the effects or response of the system to administration of a therapy, e.g., a chemical entity. The features will often evaluate over different organs or samples, depending upon accessibility and relevance of the samples. For example, in a lung disease context, samples generally considered relevant include blood, which may comprise cells, serum, or plasma; samples taken before and/or after therapy; biological cell samples, which may be biopsy, tumor, or tissue samples; fluid samples such as lavage or induced sputum samples, or postmortem tissue.

Expression evaluations need not be limited to single sample sites, but may evaluate comparative levels across relevant sample sources, e.g., blood and biopsy, or multiple organs, e.g., imaging of both liver and brain.

### B. Gene Markers

Phenotype correlation to specific genes is the subject of the science of genetics, and of the related fields of molecular biology or molecular genetics. See, e.g., Hedrick (2004) Genetics of Populations (3d ed.) Jones and Bartlett Pub., ISBN: 0763747726; Griffiths, et al. (2004) An Introduction to Genetic Analysis (8th ed.) Freeman, ISBN: 0716749394; Hartwell, et al. (2003) Genetics: From Genes to Genomes (2d ed.) McGraw-Hill, ISBN: 0072462485; Strachan and Read (2003) Human Molecular Genetics (3d ed.) Garland, ISBN: 0815341822; Lewontin, et al. (2002) Modern Genetic Analysis: Integrating Genes and Genomes (2d ed.) Freeman, ISBN: 0716743825; Klug and Cummings (2002) Concepts of Genetics (7th ed.) Prentice Hall, ISBN: 0130929980; Snustad and Simmons (2002) Principles of Genetics (3d ed.) Wiley, ISBN: 0471441805; Hartl, et al. (2002) Essential Genetics (3d ed.) Jones and Bartlett Pub., ISBN: 0763718521; Brown (2002) Genomes (2d ed.) BIOS Sci. Pub.; Nussbaum, et al. (2001) Thompson and Thompson Genetics in Medicine (6th ed.) Saunders and Company, ISBN: 0721669026; Alberts, et al. (2002) Molecular Biology of the Cell (4th ed.) Garland Pub.; Lodish, et al. (2002) Molecular Cell Biology. (4th ed.) Freeman; Haines and Pericak-Vance (eds. 1998) Approaches to Gene Mapping in Complex Human Diseases Wiley-Liss, ISBN: 0471171956; Kwok (2001) “Methods for genotyping single nucleotide polymorphisms” Ann. Rev. Genomics and Human Genetics 2:235-258; and Lin, et al. (2005) “Sequencing drug response with HapMap” The Pharmacogenomics Journal 5:149-156. Moreover, complex statistical methods are used in determining population genetics and correlations of phenotypes to specific genes.

Typically, the focus is on the correlation of phenotype to genetic elements, and typically specific alleles or other genetic haplotype markers. The correlation is measured by standard coefficients, and will typically be high, e.g., at least about 98%, 96%, 94%, 91%, 88%, 84%, 81%, 78%, 70%, 60%, 50%, 40%, etc. In many approaches, the alleles or haplotypes are represented by structural polymorphisms, e.g., which are more easily defined structurally in the form of nucleotide polymorphisms. Often the concept of Single Nucleotide Polymorphisms (SNPs) is substituted for entities which are conceived as genes, whether structural (encoding) or less well defined genetic features. The determination of genetic makeup, or the genetic details of an individual, is useful for such analyses. See, e.g., Lettieri (2006) “Recent applications of DNA microarray technology to toxicology and ecotoxicology” Environ. Health Perspect. 114:4-9; Wei, et al. (2005) “Data-driven analysis approach for biomarker discovery using molecular-profiling technologies” Biomarkers 10:153-172 (PMID: 16076730); Roelofsen, et al. (2004) “Proteomic analyzes of copper metabolism in an in vitro model of Wilson disease using surface enhanced laser desorption/ionization-time of flight-mass spectrometry” J. Cell Biochem. 93:732-740 (PMID: 15660417); Zhao, et al. (2004) “Identification of differentially expressed genes with multivariate outlier analysis” J. Biopharm. Stat. 14:629-646 (PMID: 15468756); Irwin, et al. (2004) “Application of toxicogenomics to toxicology: basic concepts in the analysis of microarray data” Toxicol. Pathol. 32 Suppl 1:72-83 (review; PMID: 15209406); Sarrif, et al. (2005) “Toxicogenomics in genetic toxicology and hazard determination: introduction and overview” Mutat. Res. 575:1-3 (PMID: 15924883); Sarrif, et al. (2005) “Toxicogenomics in genetic toxicology and hazard determination—concluding remarks” Mutat. Res. 575:116-117 (PMID: 15924887); Hamadeh, et al. (2002) “Prediction of compound signature using high density gene expression profiling” Toxicol. Sci. 67:232-240 (PMID: 12011482); Hamadeh, et al. (2002) “Gene expression analysis reveals chemical-specific profiles” Toxicol. Sci. 67:219-231 (PMID: 12011481); Hamadeh, et al. (2002) “An overview of toxicogenomics” Curr. Issues Mol. Biol. 4:45-56 (PMID: 11931569); Hamadeh, et al. (2002) “Detection of diluted gene expression alterations using cDNA microarrays” Biotechniques 32:322, 324, 326-329 (PMID: 11848409); and Hamadeh, et al. (2001) “Discovery in toxicology: mediation by gene expression array technology” J. Biochem. Mol. Toxicol. 15:231-242 (PMID: 11835620). Other polymorphisms than SNPs, e.g., those with lower than 1% frequency in the population, can be similarly valuable markers.

However, the phenotype resulting from a specific gene may be modified by the milieu of its environment, whether physical or biological. The classical Mendelian model of dominant or recessive alleles presumes that phenotypes are determined by single genes, and that the phenotype is not largely multifactorial. In contrast, multifactorial influences will more typically determine a phenotype, and the dominance or recessive feature of an allele or haplotype may be largely affected by the specific other alleles or haplotypes present, including regulatory or other functional determinants of outcome. For example, one allele may be amplified, modified, attenuated, or repressed by such other factors, many of which will be the one or other alleles present. See Yan, et al. (2002) “Allelic variation in human gene expression” Science 297:1143.

Often, alleles are considered to be defined by chromosomal location, and thus “different” alleles may be defined by alternative alleles found positionally on a chromosome. The term allele does not require that the sequence region be coding or “expressed” in a transcriptional or translational context. However, it is well recognized that occasionally gene duplications may occur, and the “duplicated allele” would then be categorized as an allele corresponding to the others. In other circumstances, there may be whole or partial chromosomal duplication (or deletion) effects, where allelic or gene dosage might be affected.

Alternatively, alleles may be entities which are highly related sequence-wise. In this case, a “mutation” would be considered an alternative allele, though they have different but closely related sequences. Thus, sequence relatedness may be another characteristic of allelic relatedness. Such alleles may exhibit, e.g., at least about 98%, 95%, 90%, 85%, 80%, etc., identity over appropriate segments. Identity may be determined using any appropriate method, including, but not limited to, the BLAST algorithm, as described in Altschul et al. (1990) J. Mol. Biol. 215:403-410 (using the published default setting, e.g., parameters w=4, t=17 as a non-limiting example).

The segments may often involve full coding region and adjacent regulatory segments, full coding region, segments of conserved sequence, e.g., domains, portions thereof, or a plurality of segments of appropriate length. Examples of polymorphisms which affect expression have been described. The segment, or plurality of segments, will typically be at least about 30, 40, 60, 80, 100 or more nucleotides, or correspond to at least about 15, 20, 25, 30, 40, 70 or more amino acid codons.

Functional relatedness may be another feature of alternative alleles. Thus, if one allele corresponds to a specific encoded enzyme (e.g., along the “one gene corresponding to one enzyme” model), another enzyme which can substitute functionally or structurally (e.g., in a multisubunit complex; as a related pharmacological binding target, or as a regulatory component) could be considered an alternative allele, even if it is not encoded at the same genetic locus. Thus, it will be useful to evaluate other allelic entities which might share substrate or reaction specificity and/or expression in similar or alternative organ or physical locations. It will be particularly useful to evaluate, e.g., for presence, quality, and/or quantity of, alternative entities which share similar substrate specificities, enzymatic turnover numbers or rates, and the like. These may include proteomic variants, such as glycosylation, phosphorylation, or regulatory variants.

Thus, the multifactorial component of phenotype actually suggests that the “combination of factors (or haplotypes)” is likely really the determinant of the end phenotype. And diagnosis of phenotype will be much more effectively achieved by determining the presence and absence, quantity, and quality of all relevant factors. Thus, correlation of phenotype to individual genes or haplotypes will be inherently less precise than to combinations of relevant haplotypes (typically referred to herein as “complement of haplotypes”). Thus, the statistical analysis goal will be to correlate phenotype to “all relevant factors”, rather than to single genes, or allelic pairings only. And the number of genes, coding regions, or discontinuous haplotype segments to be evaluated may run from about 5, 7, 9, 11, 14, 17, 21, and more. Discriminatory, classification, or substitute marker patterns diagnostic of phenotype will be identified using this process.

### C. Non-Genetic Factors: Penetrance; Environmental; Developmental; Stress; Diet; Behavioral; Medical Records

Penetrance of the “pattern of relevant factors” will also have influences. These will be factors which explain why clonal genetic systems (twins; genetically identical individuals) may exhibit variation in phenotype, perhaps for stochastic reasons. These will include, among many other things, the developmental aspects of the biological system (distant history), the recent history of the system (e.g., current environmental factors which affect the physiology or other biology, e.g., diet, stress, behavioral factors which affect the biology, hormonal factors, circadian factors, etc.), disease processes, medication processes, and other factors which affect the biochemistry, physiology, or other biological features of the relevant environment. In particular, the combination of therapeutic entities will be important, as drug-drug interactions often occur in individuals experiencing complex medical conditions.

However, since features which may be important often cannot be predicted, the best capability to have such documented are those factors which medical practitioners consider to be relevant. Thus, medical records are intended to provide the observations which have statistical likelihood, given prejudices of how biological systems operate, of explaining the phenotype. Thus, medical records are invaluable in the attempt to discover non-genetic factors which contribute to particular phenotypes. Alternatively, features which have been correlated with phenotype in other studies are likely candidates. See, e.g., Frank and Hargreaves (2004) “Clinical biomarkers in drug discovery and development” Drug Discovery 22:15-22 and related articles.

### D. Systems Biology

Systems biology is highly relevant in understanding the systemic aspects of physiology in an intact animal. In particular, most biochemistry is studied in and arguably two dimensional system, in mainly homogeneous solution with a time dimension. However, an organism has significant topological features, including intracellular organelles, internal organs, and interactions between organs which can interact via circulation and lymph. Thus, diverse regions of the body may contribute to toxicity, and also to disease. In particular, although toxicity may be manifested in a particular location, the underlying cause may be in one or several other remote organs or locations. Looking at the site of manifestation of symptoms may be looking at the wrong place.

A first application of systems biology will be to identify additional markers which are relevant to already identified markers. Pathway members upstream or downstream of an identified marker are likely candidates to also be relevant to the toxicity pathway, and are potential block points to progression or control. Pathway related entities may be found from many sources, including (1) biomarkers which have been reported to physically interact or co-localize with a candidate; (2) biomarkers which have been mentioned in a publication with a candidate (suggesting functional or structural similarity, whether a likely off-target functional or binding interaction with a therapeutic compound); (3) biomarkers which are similarly regulated in gene expression studies with a candidate in various organs, suggesting a true coordinate regulation; (4) biomarkers which are similarly localized in various organs, also suggesting coordinate regulation; and (5) biomarkers which are closely located physically on a chromosome, e.g., within thousands, tens of thousands, hundreds of thousands, millions, tens of millions, etc. nucleotides, or 0.1, 0.3, 1, 3, 10, 30, 100, 300, etc., centimorgans. Different combinations of these indicators may be used.

Alternatively, other entities known or reported to interact with a relevant marker are potential targets for regulatory intervention. Other “related” aspects of a marker may take the form of structural or functional variants of the marker, which may serve to change the kinetics or specificity of the pathway progression. Screening methods are available to screen likely aspects of identified biomarkers to evaluate DNA copy number, RNA expression levels, protein expression levels, features of the protein which are likely to affect function, including post-translational and similar modification (e.g., phosphorylation, acetylation, methylation, glycosylation, ubiquitination, etc.), or enzyme turnover numbers, half-life, and other similar features.

Existing expression data may be relevant to determine where to look. For example, many organs may be eliminated as relevant sites for analysis if the biomarker is not expressed in those sites.

### E. Temporal Analysis

Temporal dynamics in biochemistry are often poorly explored. While certain temporal dynamics are well recognized: neurobiology and ion flux changes over millisecond time spans, circadian rhythms of behavior and metabolism, menstrual cycles of hormonal changes over monthly intervals, and seasonal changes of hibernation and migration, the temporal aspects of toxicology have been little investigated. Dynamic aspects of diagnostic assays are often poorly understood, and many vary dramatically over such time periods. Gene expression profiling data will often be subject to much larger noise components than the signal, and the relative expression levels of genes may be lost in such cyclical variation. Thus, studying toxicity pathways in the context of dynamic physiology may uncover a heretofore unrecognized dimension to its understanding.

The dynamics of initiation, progression, and eruption of symptoms are not well understood. Tracking such progression, especially in a single individual, may allow identification of earmark patterns of features related to the biomarkers. Gene expression, protein expression, or metabolic function are features of likely relevance. Once such earmarks are recognized, the features may be used to monitor dynamically progression of the pathway in individual patients to monitor when the eruption of symptoms and predict timing of onset of symptoms. Management of the pathway then becomes more easily manageable, and can determine the timing of necessary actions to prevent or deal with the toxicity. Switching to a different drug or administering a preventative treatment may be in order.

Particular patterns of dynamics can be evaluated, based on sufficient time points and scales. Thus, if the progression of the toxicity pathway takes hours, the evaluation should establish baseline levels, trace it across a sufficient period of time, and probably at least follow through to full manifestation of symptoms. Sufficient numbers of analyses should be performed over the window, e.g., minutes, tens of minutes, fractions of hour, hours, fractions of days, days, weeks, months, or even years. Alternatively, time periods would be in the ranges of 1, 3, 10, 30, 100, 300, 1000, 3000, 10K minutes. Optimally, the manifestation of symptoms is sufficiently separated from earmarks that a monitoring system can identify with reliability, allowing identification of earmarks indicating onset of irreversible progression.

Typical dynamic patterns will encompass, e.g., constantly steady, steady change (increasing or decreasing), increasing and then decreasing, decreasing and then increasing, stable then changing, changing then stable, with time points for inflection being particularly notable. Differences between patterns characteristic of clinical phenotypes are of greatest interest. Often earmarks of events include a combination of patterns of different biomarkers.

### F. Population Homogeneity: Genetic; Environmental; Behavioral; Recordkeeping

As discussed, the penetrance of a defined genetic state is affected by genetic or non-genetic factors. The statistical analyses which can identify meaningful genetic features will be most successful where interfering noise is minimized, i.e., where identification of false positive factors or false negative factors will be minimized. The elimination of population heterogeneity will maximize the opportunity to recognize the signal over noise. Thus, analyses will be greatly improved and will be mathematically most efficient when performed on a homogeneous population of sufficient size. In contrast, the population should exhibit sufficient heterogeneity that the spectrum of phenotypes contained therein is reflective of a “global” population.

Studies in humans would be preferred, which eliminates issues of whether a non-human mechanism is relevant to a human. In human genetics studies, some compromise should be selected between a homogeneous population whose phenotypic variation totally fails to reflect mechanisms which may be present in other populations, and another extreme of a highly diverse a population where the non-genetic factors are such that the penetrance of the genetics might fail to be discernable. Statistical methods in genetic analyses are described, e.g., in the references on genetics described above, and in Ewens (2004) Mathematical Population Genetics (2d ed.) Springer, ISBN: 0387201912; Fernholz, et al. (eds. 2001) Statistics in Genetics and Environmental Sciences Birkhauser, ISBN: 3764365757; Svirezhev and Passekov (1990) Fundamentals of Mathematical Evolutionary Genetics (Mathematics and its Applications) Springer, ISBN: 9027727724; and Halloran and Geisser (eds. 1999) Statistics in Genetics (IMA Volumes in Mathematics and Its Applications) Springer-Verlag Telos, ISBN: 0387988289. Studies using genetically homogeneous populations are described, e.g., in Shifman and Darvasi (2001) “The value of isolated populations” Nat. Genet. 28:309-310; Kruglyak (1999) “Prospects for whole-genome linkage disequilibrium mapping of common disease genes” Nat. Genet. 22:139-144; and similar publications.

Homogeneous and/or large population sources for genetic studies are useful, preferably with medical details allowing subsetting, e.g., East Finland Population (Jurilab); Icelandic population (deCODE Genetics); Ashkenazi Jew population (see, e.g., familystudy@jmhi.edu; or Johns Hopkins School of Medicine); Sardinia (Shardna Life Sciences); Quebec (Genizon Biosciences); Mormon population (Utah Population Database (UPDB) and with Church of Latter Day Saints (LDS) records); Estonian Genome Project (EGen); Iranian Human Mutation Genebank and database (see Najmabadi, et al. (2003) Hum. Mutat. 21:146-50); UK Avon Longitudinal Study of Pregnancy and Childhood (ALSPAC); Costa Rica (see Service, et al. (2001) Hum. Mol. Genet. 10:545-551); Newfoundland (Newfound Genetics); and others. Features of particular interest to genetic homogeneity include, e.g., geographic isolation, homogeneity, founder effect, genetic drift and extended linkage disequilibrium (LD). See also Rahman, et al. (2003) Human Molecular Genetics 12:Review Issue 2 R167-R172; and Heutink and Oostra (2002) Hum. Mol. Genet. 11:2507-2515.

The homogeneity of a population is often difficult to define quantitatively, and the sampling is subject to difficulty in definition by inclusion of outliers, whose data may be disregarded or eliminated later based on results of more careful analysis indicating genetic outsider status. But the invention allows, by testing, means to confirm by SNP or other methods criteria for inclusion into the desired population. Although there are different ways to mathematically define linkage disequilibrium, it relates to how much of the genome is typically coordinately inherited. The means to define such are based upon the selection of markers to evaluate such, generally polymorphisms, generally referred to as single nucleotide polymorphisms (SNP), in nucleic acid sequence of the genome. With a selected set of SNPs, there are measures of the granularity of analysis, e.g., how homogeneous is dispersion of the markers. Both can be evaluated by a quantitative measured of “median” or “mean” intermarker distance. The ranges are typically in the thousands of KB separation, while high throughput microchip technology can provide generally from about 90K to 500K SNPs on a single sample analysis. With such resolution, and the size of the human genome, one gets about 4 KB mean separation. But based on the specific set of SNPs used, the median range may vary depending upon what regions may have higher or lower density SNPs selected.

The values for linkage disequilibrium range from 0 (no unusual linkage) to 1 (highest linkage), and may run in a range from 0.20, 0.25, 0.30, 0.35, 0.40, 0.45, 0.50, 0.55, 0.60, 0.65, 0.70, 0.75, 0.80, 0.85, or 0.90 depending upon the granularity of regions being evaluated. Higher local LD values over greater distances are more significant than higher LD values over shorter distances. See, e.g.,; Shifman, et al. (2003) Hum. Molec. Genetics 12:771-776. The regions of greatest interest in these analyses will be specifically local physically to biomarkers discovered as described, e.g., in Tables 3 and 4 directed to liver toxicity markers.

As such, analyses of populations may also take the form of selecting amongst the data derived from subsets within the populations, with the “most homogeneous populations” being selected subsets of populations considered genetically homogeneous, e.g., with selection excluding individuals identified as being not within the characteristics defining the homogeneity.

A preferred population will be one comprising a founder population having a low number of founders, is traceable back through several generations (preferably at least about 5, 8, 12, 15, 20, or more), and will have comprehensive medical and historical information (preferably some medical records for most, other information on genetic relationships, e.g., church marriage and parentage records), a high rate of “inbred” population expansion, and be large enough to allow for sizable study cohorts. The number of founders preferably is less than about 20K, 15K, 3K, or even about 1500 or 900, often determinable by evaluating mitochondrial DNA or Y chromosome homogeneity. The number of substantially traceable generations preferably will be more than about 5, 10, 15, 20, 50 or more generations. Useful medical records preferably will exist and/or be available for at least 5, 10, 15, 20, 25 or more years, and the familial relationships substantially traceable for 3, 5, 7, 10, 13, 17, or 20 generations. The details of the medical records will range from limited, occasional events, e.g. only hospital admissions, to more frequent clinic visits; and details may range from complete medical records with associated diagnostic test results to limited annotations relating to sex, age, outcome, or the like. Often the particular sample itself provides annotations, e.g., sex organs may inherently subset, or can be readily determined by simple diagnostic procedure on the sample (e.g., presence of Y chromosome). For statistical purposes, the study population derived from the founders will preferably be at least about 70K, 140K, 220K, 300K, 500K, 800K, 1.1 M, 1.5 M, or more, and the phenotype numbers will preferably be large, e.g., at least about 5, 7, 10, 13, 16, 20, 25, 50, 100, 150, 200, or more examples. Adverse event reporting schemes may identify reports of at least about 5, 10, 20, 30, 50, 70, 100, 150, 200, 450, or more putative events.

In particular, genetically homogeneous populations allow for generation of hypotheses of correlations exhibiting low false positive rates, e.g., providing advantageous statistical power. Validating those hypotheses among larger and more heterogeneous sample populations is much simpler and less costly than doing the initial studies in the larger and more heterogeneous population. Statistical power in the latter population is poorer and will lead to many more false positives and spurious signals among the higher background noise statistics.

Alternative means to access samples for evaluation include clinical trial biobanks of sufficient size that phenotypically homogeneous cohorts can be selected. Those biobanks may be derived from clinical trial samples, or outside of a clinical trial context a large enough collection of relatively non-homogeneous samples but with sufficient annotation to select relevant cohort subsets. Biobanks, also known as human tissue banks or biorepositories, include various governmental efforts including the UK, Estonia, Canada, Norway, Sweden, US (including NIH), Iran, Singapore, Japan, Spain, Germany; private biobanks, e.g., deCODE, Oxagen, Galileo; tissue biobanks, e.g., Ardais, Genomics Collaborative, Astand, ILSbio; and disease focus banking efforts, e.g., heart disease, osteoporosis, bone marrow registries, breast cancer registries, foundations (cystic fibrosis, Parkinson's disease, etc.). See, e.g., Zimmerman, et al. (2004) Biobanks: Acclerating Molecular Medicine, IDC #4296; International Society of Biological and Environmental Respositories (www.isber.org); and International Biobank and Cohort Studies Meeting, Feb. 7-8, 2005, Atlanta, Ga. Alternative sample sources may include national, e.g., UK, Canadian, or China health care systems, Health Maintenance Organization samples (e.g., medical records associated with patients with permission to sample), health insurers, or medical centers.

The homogeneous human cohorts may be used either in a training subset, to generate a hypothesis, and/or to validate a hypothesis. The former is often much more difficult, as it makes fewest assumptions about the biology leading to toxicity pathway activation, while the latter can confirm a hypothesis which may have been generated from any source, e.g., animal model data. The number of cohort samples for the training set and/or validation set (separately or in combination) from the genetically homogeneous population is preferably at least 5, 10, 15, 30, 45, 65, 80, 100, 120, 145, 170, 200, 235, 270, 300, 330, 370, 410, 440, 470, 500, 600, or more.

### III. Correlated Genes to Pathway Hypotheses

The initial identified gene or haplotype dataset will identify genes believed to be genetically correlated with phenotype. With tools which allow, e.g., cross species comparisons of structure and presumptive function, the species from which the gene dataset is derived may be different from the species in which the biomarkers are desired. From the understanding of function or pathway networks (pathways and networks will typically be used interchangeably) in one species, structural correlation across species, and functional studies may be used to cross species boundaries. Thus, a gene identified in a rat species dataset would often be expected to have human counterparts, either structurally or functionally. This sets up a hypothesis which can be tested in a human based system, directly or indirectly. Typical pathways likely to be relevant to toxicology will include detoxification pathways (e.g., cytochrome P450s), transporters (influx/efflux), drug metabolizing pathways, and the like.

From a set of identified genes which are believed to be correlated with phenotype, the assortment of genes will point to pathways/networks of interacting genetic entities which relate to the phenotype. Many of the genes will be readily assigned to pathways. Most others can be assigned by use of ontogeny analyses, which will allow the identification of less clearly understood pathways, networks, or mechanisms. The few remaining genes which do not readily get assigned to a network or mechanism can be analyzed using systems biology and cross species structural and functional means to assign probable pathway linkages. This provides the means to apply datasets from one species, e.g., mouse, rat, dog, or primate, to another, e.g., human. However, the identification of a marker (or series thereof) in one specie would need to be applied to counterpart markers in another specie in the context of the pathways and physiology of that second specie.

These pathways and networks are then evaluated, with respect to its members, for the functions which are necessary for the pathway. These will include the development of the structural components, creation and regulation of the various components, and maintenance of the other functional features of the pathway or networks; eachof which relate to the phenotype.

With the identification of the components of the pathways or networks, hypotheses as to which components are critical points which would regulate or control the development or prevent the appearance of the phenotype. These hypotheses will then be testable to identify combinations of genes or biomarkers which contribute to the phenotype. Perturbation analyses and surrogate markers can be applied to determine which biomarkers possess maximum relevance to the phenotype.

### A. Gene Sets to Pathways and Networks

Within the identified sets of genes whose expression is correlated to phenotype, many of the genes will be readily assignable to understood metabolic pathways or networks, and it will often be readily understood how that pathway can mediate the resulting phenotype. This identification can have a dramatic impact on recognizing that different pathways or networks have relevance in the phenotype which had heretofore remained unrecognized. Systems biology interactions between networks and biological systems will become better understood as the relevance of a pathway will be seen to impact seemingly remote phenotypes, e.g., how fundamental metabolic pathways or networks have impacts on multiple organ systems, or how pathways or networks recognized to affect one system actually also impact phenotype in the another system or remote body location. Conversely, many different pathways may affect the same phenotype in similar or related ways.

However, among the identified marker set, some fraction will not be readily assigned to or within a known pathway or network. In these situations, a predictive model will typically be developed in two steps. A first step might be characterized as identifying or developing an initial limited set of “preliminary” signatures for specific data types. A separable second step might be characterized as generating a predictive model that combines different types of signatures, and may incorporate more discriminating and higher resolution features which correlate more closely with the desired prediction.

As an example of a step I, consider gene expression measurements for several thousand genes (or, similarly, a proteomic profile, a metabolomic profile, or a mixture or combination of various forms of profiles) for each sample. An initial step might be data reduction, e.g., intending to focus on and identify a profile of few genes or features that account for a majority of the variation in the data. See, e.g., Joliffe (2002) Principal Component Analysis (2d ed.) Springer, ISBN: 0387954422; Krzanowski (2000) Principles of Multivariate Analysis: A User's Perspective (Oxford Statistical Science Series) Oxford Univ. Pr. ISBN: 0198507089; Gnanadesikan (1997) Methods for Statistical Data Analysis of Multivariate Observations (2d ed.) Wiley-Interscience, ISBN: 0471161195; Ramsay and Silverman (1997) Functional Data Analysis (Springer Series in Statistics) Springer, ISBN: 0387949569; Muirhead (1982) Aspects of Multivariate Statistical Theory (Wiley Series in Probability and Statistics) Wiley-Interscience, ISBN: 0471094420; Mardia et al. (1980) Multivariate Analysis (Probability and Mathematical Statistics) Acad. Pr., ISBN: 0124712525; and other texts on multivariate analysis.

One approach is to use Principal Component Analysis (PCA) to reduce the dimension of the data. The first few components (e.g., select the top 5) may account for a majority (say 90%) of the data. As one adds more components, the modeling improves, but the marginal improvement in the model will decrease with each additional component included (decreasing return).

Another approach is to group the data using a clustering technique. Many such techniques exist, with the objective to bin the data into clusters so as to maximize distance between clusters (e.g., to distinguish each cluster from its neighbors). See, e.g., Anderberg (1973) Cluster Analysis for Applications Academic Press; Hoppner, et al. (1999) Fuzzy Cluster Analysis Wiley, ISBN: 0471988642; Zhao (2004) “Evolutionary Computing and Splitting Algorithms for Supervised Clustering” Masters Thesis, U. of Houston (http://www.es.uhu.edu/˜zhenzhao/zhenghongthesis.zip); and Dettling and Buhlmann (2002) “Supervised clustering of genes” Genome Biology (2002) 3:1-15. Clustering techniques vary widely, and some are more successful than others at separating the various clusters.

Yet another approach is to use supervised clustering techniques. The objective here is to classify data using information other than what is contained in the data itself. For example, knowledge about a group of genes regulated by the same transcription factor can be used to group them together; another is to use medical record data to group data.

The end result of a step I might be an identified handful of genes or patterns accounting for most of the variations contained in the larger dataset. The patterns may be combinations of different markers, different forms of analyses (genotypic, RNA expression, protein expression, post-translational features, and/or metabolic features), different sites or organs, temporal features, and such. See, e.g., Abraham (2004) Trends in Biotechnology 22:15-22; and Frank and Hargreaves (2003) Drug Discovery 2:566-580.

In a step II, a predictive model may be built that combines several of these patterns. This might be characterized as a curve fitting exercise. The end point is the desired clinical outcome correlated back to a pattern or signature.

In one approach to this development of a predictive model, one can work backwards. For example, one may say that if a particular patient (data point) has genotype pattern X, expression profile Y (Y could be a combination of several distinct profiles, e.g., proteomic, or metabolomic), and clinical phenotype Z, then there is an n % chance (correlation coefficient) of toxic event. One simplistic modeling strategy would construct a model with X, Y, and Z as parameters with proper weight factors (correlation coefficients) to fit the population of data points. The weights will be chosen (assigned) to account for the known clinical outcome across the data points.

Alternatively, another approach would be the use of a neural net. See, e.g., Pearson, et al. (eds. 2004) Artificial Neural Nets and Genetic Algorithms: Proceedings of the 6th International Conference in Roanne, France, 2003 Springer, ISBN: 3211007431; Kurkova (ed.), et al. (2001) Artificial Neural Nets and Genetic Algorithms Springer, ISBN: 3211836519; Bishop (1995) Neural Networks for Pattern Recognition Oxford Univ. Pr., ISBN: 0198538642; and Nelson and Illingworth (1994) Practical Guide to Neural Nets Addison Wesley Pub., ISBN: 0201633787. In some such embodiments, the network is trained with part of the data; that training leads to a hypothesis; and the hypothesis is cross-validated against the rest of the data.

Yet another modeling technique to generate a predictive model is the use of decision trees. Here data is split iteratively in a tree like form with subsequent branches explaining more and more of the data and ultimately reaching a class. See, e.g., Mitchell (1997) Machine Learning McGraw-Hill, ISBN: 0070428077; and Duda, et al. (2000) Pattern Classification (2d ed.) Wiley-Interscience, ISBN: 0471056693.

Having applied the various alternatives here, the end result of a step II would be a model (or hypothesis) that weighs the inputs to construct a predictive model relating to a form of related network or pathway. This model would be constructed using part of the data (“training set”); while other parts of the data are used to validate the model.

Yet another means to reach understanding of a pathway will be based upon genomic information, evolutionary phylogeny, and related systems biology. It will often occur that a gene of unknown function has structural, sequence, or regulatory similarity to another in a different species. This may provide insight and testable hypotheses relating to the function or pathway in the original species, or other species may provide greater details into what other features or structures may exist in a pathway or network. Thus, knowledge databases linking data within a species or across species will be useful. There are many such databases, e.g., products similar to those provided by Ingenuity, Entelos, BioVista, Jubilant BioSystems, and the like. Similar offerings should be available from the NCBI, various European or other counterparts, or other websites. Various similar pathway collections should be directed to pathways relevant to specific disease states, physiological conditions, or biological subsystems (e.g., cardiovascular, digestive, respiratory, hormonal, etc.).

### B. Pathway Identification and Elucidation

Identified genes may be correlated with phenotype (e.g., treatment outcome) to identify pathways, but individual genes or features within that pathway will each exhibit differing correlation coefficients from other features. The outcome correlation with such features may vary depending upon frequency of the feature in the selected study population, the frequency of various mechanisms of phenotype outcomes, the number of different mechanisms leading to a similar phenotype, and many other factors. However, preferred biomarkers are those which represent the most common mechanisms or pathways leading to the relevant phenotype, and within each of those networks, those biomarkers which exhibit optimal (e.g., high) correlation coefficients among various alternative markers (gene or otherwise) in the networks. Thus, optimal biomarkers or signatures may involve a plurality of different diagnostic measurements, and may include dynamic features.

Often, understanding of the pathway or network allows identification of features which are diagnostically most relevant to the phenotype, and may allow for identifying parameters and features which are directly relevant to the timing, severity, and progression of the phenotype. Thus, a form of reverse engineering will provide for generating a diagnostic strategy to fit the phenotype, and selecting the appropriate diagnostic parameters within that context.

In other circumstances, less about the pathway is known, and some experimental component may be useful to determine what features or factors are more directly relevant to the phenotype. In other situations, the pathway may be incompletely understood regarding relevant biomarkers, or the interactions or regulatory processes in its physiological function. These can be filled in by some combination of metabolic pathway analysis and systems biology analysis. The basic means to elucidate metabolic pathways follow the principles of elucidation performed in intermediary metabolism decades ago. Systems biology strategies have been referred to above. The pathway elucidation generally need not be performed in any specific species, as most pathways are shared across evolution, and the relevance of related species will typically be great. Thus, the proper species will often be selected on the basis of peculiar biological features in a species, access to tools, materials, and ease in confirming the relevance of that species to others. Fundamental pathways, e.g., those that mediate cell division, signal transduction, or processing of xenobiotics, are often conserved across the animal kingdom and in many cases between fungal and animal kingdoms as well.

The correlation between pathway or network specific features and phenotype will typically be most robust when the measurement directly evaluates a critical signature which is proximal to the phenotype (outcome result), and not a distant one. Being proximal, there will typically be fewer, if any, compensatory factors which can attenuate the end phenotype, and thus the statistical correlation coefficient will be highest (optimized). In addition, certain threshold level features, sensitivity and consistency in measurement within and across individuals and time, sampling/measurement errors, ease and speed of determination, local or centralized analyses, and other features will be technically preferred, providing cleaner measurement signal relative to noise. Other factors for selection of specific features may include economic efficiency of evaluation. As indicated above, combinatorial features will often be much superior to individual features. Temporal features may be identified, which may provide useful insight into the progression and dynamics of phenotype emergence.

Thus, analysis of the genes which correlate with a phenotype can lead to identification of mechanistic pathways which seem to be common, and thus lead to hypotheses as to mechanisms of the phenotype. Testing these hypotheses can be done much more readily with more favorable statistical precision and experimental design than creating a hypothesis from raw data, and various tests to eliminate particular models might be possible from historic or simple experimental procedures. Thus, initial identification of correlated genes to a phenotype (e.g., treatment outcome) will often be a first, but incomplete step to defining and understanding a mechanism to explain such treatment outcome (which may be an adverse drug event).

### IV. Hypotheses to Optimized Signatures

Once gene sets have been identified as correlated with a particular phenotype, analyses of those sets will point to particular pathways which mechanistically should relate to the phenotype. Thus, when the set contains many of genes within a particular functional pathway, the data strongly points to that pathway as an important component causing the phenotype. Typically, a phenotype may be caused by multiple alternative mechanisms, perhaps related or unrelated, but the collection of those mechanisms should teach the various alternative means or pathways which can lead to the phenotype.

Systems biology analysis techniques are then applied to the collection of genes identified by correlation analyses to lead to identification of those pathways. And as much as is known or can be presumed about the pathway itself can be collected. Analysis of those pathways, and understanding the networks of interactions and regulation, will reveal genes and reactions which are critical in the pathway leading to the phenotype. From the defined networks of genes and interactions will be identified features and signatures which can be hypothesized to contribute to the dynamic or temporal development of the conditions resulting in the phenotype. These hypotheses can then be tested, thereby providing signatures (single or combinatorial) with optimized correlation with outcome (particularly including significant genetic contributions), a desired temporal prediction (long before, intermediate, or immediately preceding) relative to phenotype, minimal noise, maximal diagnostic stability, high discrimination, and other desired features.

One means of testing can be by specifically applying diagnostic procedures to monitor a relevant system. Thus, an experimental system designed or recognized to involve the designated pathway may be evaluated to determine which are the bottleneck points or critical points for system stability. In vivo experimental models may be used, and often there exist in vivo models which represent a disease. In certain cases, surrogate markers may be used instead of phenotype readout, e.g., in humans, where ethical considerations may prevent direct observations of a phenotype or progression thereof.

Alternatively, in vitro models which may have surrogate markers may be used. However, the systems biology component will also be useful in pointing to what sample types (e.g., which organ or histological type) may be relevant to the phenotype exhibited by a different organ or system of the animal. The insights provided there may often lead to looking for the markers at a different location from where the phenotype first manifests observed symptoms.

Yet another method will involve experimental perturbation analyses to identify those biomarkers (and allelic variants) which can provide diagnostic measures exhibiting high correlation with phenotype.

In addition, the pathways may be evaluated to determine the main pathways which might cause effects which are manifested in the main organ systems of interest in clinical pathology or treatment. Among those systems, e.g., in the toxicology field, are the digestive, circulatory, respiratory, nervous, endocrine, homeostatic, skin, musculoskeletal, blood, urinary, and reproductive (male or female) systems. The organs comprising such systems can also be defined, e.g., in the area of toxicology the main organs of focus are liver, muscle, GI tract, bone marrow, CNS, respiratory, circulatory, and reproductive systems. Using systems biology analyses, it will become apparent that particular mechanistic pathways may cause phenotypes in different functional systems, and the site or type of manifestation of initial symptoms may depend on certain features which will be determined, as before, by genetic, environmental, physiological, or other factors, depending upon the individual. However, those factors will likely be identified only if they are features which could emerge from the medical record of a particular patient.

From the correlation of genes with phenotype, analyses of the genes will be performed to determine the functional roles of such genes. Typically, the genes can be characterized using software, e.g., Gene Ontology (CNIO bioinformatics unit), as being involved in different functional networks, and categorized among biological process, molecular function, or cellular component dimensions. For example, the genes may be involved in metabolism (e.g., enzymes, or regulation of enzymes and metabolic pathways), cellular physiological processes, cell communication, response to stimulus, regulation of physiological processes, organismal physiological process, morphogenesis, regulation of cellular process, death, cell differentiation, homeostatis, growth, protein synthesis, etc.

Studies in non-human species will provide enormous insight into the pathways which can be applicable to human phenotype. For example, studies on toxicity in rodents can, with these analyses, provide information as to what are important toxicity pathways in those rodents. With the genomic information available today, identification of relevant pathways in the rodent can be tested for equivalent relevance in a different species, e.g., human. The hypothesis that the same pathways are relevant in the second species can be then validated. And the corresponding species counterparts are reasonably easily determinable, especially with so much sequence data available, to allow validation of the hypothesis.

Identified genes which correlate with phenotype should cluster in relevant pathways, often networks of functional or structural features which relate to one another. Means to determine the function of genes can be derived from literature reports, genetic mapping studies, and others. Appropriate databases which link such include the Ingenuity, Entelos, Biovista, and Jubilant Biosystems knowledge management system databases, as described above. Descriptions of database offerings are available from simple interne searches.

With the identification of relevant pathways, identification of the specific “critical” factors or signatures, alone or in combinations, can be effected by perturbation analyses. Perturbation analysis will allow determination of whether a particular component is close to or remote from the critical phenotype determining parameter.

Large scale experimental perturbation analyses can be performed to identify those biomarkers which can provide diagnostic measures which provide high correlation with phenotype. Perturbations to proposed markers (and genetic variants) can be effected in an experimental system. Some modifications can be introduced in gene sequence or quantity (regulation); protein sequence, modification, or quantity; substrates or analogs thereof (including inhibitors or regulatory subunits); metabolic intermediates; time of endpoint or analyses; temperature; and isotopic variants. Other changes can be achieved by any of gene expression modifiers (including knockout or transformants), gene suppression (e.g., using RNAi or anti-sense), use of dominant negative forms or suppressors, and activating mutants. Some perturbations may be chemical perturbations, e.g., by varying concentrations of small molecule inhibitors, co-factors (natural or otherwise), or activators, or perturbations in measurements as a function of time. See, e.g., KineMed (www.kinemed.com), in which kinetic features are studied in fundamental problems in disease management and drug development.

Although it is widely recognized that biological systems are characterized by flux (polymers are synthesized and degraded; metabolites traverse pathways to provide energy; cells die and are replaced), virtually no tests in biomedicine currently measure dynamic fluxes of molecules. Contemporary drug development/diagnostic strategies ignore this approach, analogous to the state of photography before the development of moving pictures, or of moving pictures before the introduction of sound.

This view rests, in part, upon the hypothesis that the operational unit of function in complex biological systems is neither the gene nor the protein, but rather is the flow of molecules through metabolic pathways in fully assembled living systems. In the final analysis, it is the flow of molecules through synthetic, catabolic, and intermediary metabolic pathways that is responsible for phenotype.

For example, assays may combine highly sensitive mass spectrometry and the labeling of critical molecular pathways with stable, non-radioactive isotopes in living organisms, including humans. The development of this technology allows for the measurement of molecular fluxes in metabolic pathways critical to human health and disease. The stable isotopes can be delivered by many routes of administration, are safe for use in humans, and the isotopic enrichments of a number of metabolic pathways may be determined in a high-through put manner. These kinetic assays have been broadly applied to a vast array of human disease states.

Perturbation analysis is described, e.g., in Jansen (2003) “Studying Complex Biological Systems Using Multifactorial Perturbation” Nature Reviews Genetics 4:145-151; Bowr and Bolouri (2001) Computational Modeling of Genetic and Biochemical Networks MIT Press; Kanji (1999) 100 Statistical Tests Sage; Collado-Vides, et al. (1996) Integrative Approaches to Molecular Biology MIT Press; Adriaans and Zantinge (1996) Data Mining Addison-Wesley; and Everitt and Dunn (1991) Applied Multivariate Data Analysis Arnold.

### V. Optimized Signature Relevance to Humans

The optimized signatures may be in humans or non-human species, but will often be essentially surrogate markers for the phenotype. Where the signatures in the model systems have not been directly demonstrated in human systems, validation must be performed. However, given systems biology analyses and genomic data, the optimization might be performed in a non-human or quasi-human context. Further studies necessary for conversion of those signatures into the corresponding human systems may be minimal.

However, the methodology may also work backwards to establish that certain experimental systems can be directly relevant to humans with accepted surrogate markers. When it is established that an experimental system is diagnostic of whole organism human phenotype, the experimental model then can be used to test candidate therapeutic treatments or entities. The “experimental” feature then allows one to test new clinical candidates, rather than being limited to using approved entities in humans for determining phenotype, e.g., therapeutic response.

The experimental systems may be, e.g., in vitro or in vivo, and can be genetic, developmental, physiological, or other systems useful as models of disease or conditions. The models may be based upon the correlation of the optimized signatures back to the similar signatures detected in humans in a whole organism context, where the various functional or structural systems are intact and interacting. This can lead to surrogate markers or signatures applicable to experimental systems, but which are linked to intact human outcomes.

Additional input will derive from computational modeling (e.g., combining chemical structure with biological outcome; e.g., Leadscope technology, Multicase, DEREK, and the like), which will include data mining, structural alert determination, statistical correlations, fragmentation of chemical structures, expert rules, and the like.

Thus, cell lines or systems may be used, including alternative species, or human cell lines. The cell lines or systems may be human, transformed, transfected, or modified to exhibit features characteristic of the human phenotype, including features of human disease or pathological conditions. The cell lines or systems, including derivatives from stem cells, will generally be designed to provide readable signatures, as identified using the processes described, which can provide useful correlation back into the intact human systems. And the curve fitting component of the model building inherently relates back onto intact human data, with medical records and clinical input. Consensus biomarkers can be selected from the lists of markers from the various tables, datsets, and subsets. Particular markers can be selected which are either conserved across different datasets, or are relevant to common mechanisms of toxicity manifesting symptoms among multiple organ systems. For example, certain liver markers evaluate cell types found in the liver, e.g., PBMC, mucosa, or other cell types found also in other sites. Thus, conservation of markers may reflect (a) similar pathways operating in different target organs and/or (b) evaluating markers in different sites may actually be evaluating cells which are commonly found in the both sites. Similarities across samples, e.g., in physiology, function, structure, gene expression, and/or developmental origin (e.g., ectoderm, mesoderm, and endoderm), should often reflect similarities in metabolic pathways and potential for mischief. Thus, gene expression similarities will typically cause similarities in biochemistry and toxicology responses. For example, highly vascularized tissues (which may include liver, kidney, and many immune organs such as the spleen, thymus, bone marrow, lymph nodes) will typically show significant overlap in gene expression and physiological responses to contributing vascular components such as blood, vessels, and related mucosa. Likewise, quickly growing tissues such as bone marrow, immune organs, gastrointestinal tract, and skin will commonly express cell division related pathway components. Thus, biomarkers relevant to one organ would often be expected to be relevant to other organs sharing similar physiology, function, or gene expression of relevant or related pathways.

### VI. Optimized Biomarkers/Signatures; to Cell Lines, Experimental Models

Once signatures are identified, as described, those signatures may be applied to organs or subsystems. The subsystems may include, among many, ex vivo organ or system studies, in vivo non-human organ models, cell lines or collections thereof, e.g., whose physiological or biological outcomes may simulate the range of population diversity, robotic or parallel assay methods, including “laboratory on a chip” systems for testing parameters (as identified within the signatures), and other means to test the range of responses to treatments.

The result of developing experimental models with surrogate markers and endpoints will be systems which allow for testing, screening, and accurate outcome prediction of high cost clinical studies from simpler and better correlated models. Instead of spending tens of millions of dollar at later stages of drug candidate development, reliable experimental feedback at earlier stages will allow prioritization of competing clinical candidates for few clinical development program slots. And reliable feedback early in the process will increase the success rate of candidates entering into the pipeline.

Moreover, with the development of classifier marker signatures, evaluation of test candidates for phenotypic outcome can be more easily performed. This may be applicable to testing new intervention therapeutics, or to develop more targeted toxins, e.g., in targeting tumor physiology. General environmental toxicity may be addressed using primate or other species.

### VII. In Vitro and In Vivo Disease Models

Beyond the capability of using the experimental models for evaluating phenotype, such as toxicity, the models may be developed with specific disease or medical conditions as targets. The impact from the disease state will also be incorporated into the system so that the readouts are taken in the context of the biology and physiology existing in the clinical condition. There will be enormous advantages in performing the assays in the models simulating the context of the desired target biology.

Thus, where there are genetic or physiological models of the clinical condition, the biomarkers will be useful both in helping to determine the relevance of the proposed models and in evaluating such models to determine what treatments have positive effects on the “surrogate” markers. Various technologies may be utilized to evaluate where and when various metabolic systems are important. Imaging methods may be developed to evaluate signatures internally at selected sites to monitor or otherwise identify either adverse responses or to monitor disease development or progression.

It will be recognized that similar methods may be used to focus on mechanisms of specific diseases or conditions, and is not necessarily limited to application to liver toxicity mechanisms. Thus, the methods may be used to study defined phenotypes, whether classified together by medical practice, or clustered together by molecular definition of condition. The ultimate goal is molecular definition of conditions, distinguishing different mechanisms leading to similar symptoms, and reaching the possibility of personalized treatment of defined conditions.

### VIII. Output Products

### A. Methods and Diagnostic Platforms, Tools for Therapeutic Applications

Fixed time point diagnostic products should result from identification of markers, e.g., from non-humans, using genomic data and systems biology to point to human gene, protein, and metabolomic counterparts. Extension from single markers to multiple combination markers, e.g., including evaluation of attenuating factors derived from further genetic correlations, medical records data, non-genetic markers, disease or medical condition factors, behavioral or life style factors, and other correlations will lead to better quality and higher accuracy diagnostic packages. Development of hypotheses in non-human species, along with access to human population phenotype or outcome endpoints will assist in generating hypotheses which will lead to efficient statistical and cost effective validation for diagnostic products and methods. Human studies will also allow identification of new biomarkers and products derived there from.

Dynamic (multiple timepoint) monitoring should result from recognition of the dynamic signatures culminating in phenotype, both immediate and distant future events. This understanding should allow for continuous monitoring diagnostic devices and methods to track the progression of risk development over time. This, combined with the combinatorial analysis, should provide important new perspectives on how diagnostic strategies and patient monitoring will be performed.

Methods and constructs utilizing the biomarkers and pathway and network information will be incorporated into experimental systems. These will be incorporated into in vitro and other assays for testing treatments. Classifier or substitute measures for phenotype will result, along with better refined measures coming from focus on critical, robust, and combinatorial signatures. Dynamic factors will be better understood, and predictive methodologies will be developed.

With the complex combinatorial and dynamic biomarkers identified, as described, adoption into models of disease or condition can be better monitored for signatures correlated to phenotype. Thus, surrogate signatures may be validated and become acceptable means to use experimental subsystems, in vitro systems, or in vivo components for acceptable phenotypic readouts. Such experimental systems may often incorporate human components, e.g., genes, regulatory structures, etc., or be based upon human systems, organs, tissues, or the like, with other components from animals. Mechanical systems may be incorporated to evaluate titrations over time or concentration. High throughput screening or testing systems will evaluate optimized surrogate signatures to provide useful information on human response, or to identify dangers to carefully monitor in the whole animal or human organism context.

Alternative systems include animal cell lines or systems as models for animal testing. Certain ones may incorporate human components, e.g., genes identified as critical points, to evaluate factors in human biomarker interaction. Certain animal disease models can incorporate human features, and ultimately human disease models may be generated, e.g., based on genomics and systems biology. Counterpart human tissue systems or in vitro cell systems may be combinatorially combined to develop information on the behavior of the human systems of relevance to the human disease or medical condition. These systems, alone or in combination, will lead to signatures useful for diagnosis, monitoring, or surrogate readouts for phenotype.

Besides screening or testing, these systems will also be useful for evaluation of therapeutic index of treatments. The treatments may be tested in combinations, thereby providing useful insights into combination drug interactions. As many current drug problems result from peculiar interactions of multiple drugs, these systems provide experimental means to evaluate or model, with some statistics, outcome phenotypes. These will be early attempts at providing useful experimental models and biomarkers useful to model disease situations.

### B. Therapeutic Products

These means for evaluation of phenotype, e.g., system interactions shall lead to applications directed to monitoring individuals undergoing treatment. Thus, knowing contributing genetic factors contributing to a particular phenotype may allow subsetting of patients (categorizing of patients in subsets) or potential patients into those exhibiting low or higher risk from treatment, and even to predict timing of onset of problems. This will be useful in the therapeutic context in determining what alternative treatments would be indicated, when they should be applied, and/or when danger has subsided from primary treatment so return from an alternative is safe.

In addition, the present invention provides means to evaluate or prioritize early drug candidates for clinical success. Early determination of risk for efficacy and toxicity of a drug candidate is important in prioritizing resources for pipeline drug development. Because of the enormous costs of preclinical and clinical testing, any reliable information early on can be critical in early decisions on which candidates to continue and which to terminate, or prioritization among alternatives. Because late termination is so costly, both economically and in lost time, many drug development programs are abandoned after failure of the top (first) candidate. Second or subsequent candidates often never progress even to the preclinical testing required for market approval, and thus are eliminated from the potential pharmacopeia. Thus, reliable indications of outcome of these tests can raise or lower a particular candidate among alternatives in a development program. The present invention will allow this.

Moreover, with the more robust diagnostic signatures indicating phenotype, or outcome, the invention allows for means to rescue drugs at risk for market withdrawal. If accurate and reliable diagnostic signatures can be identified which subset patients or potential patients into low and high risk sets patients for treatment, adverse drug events may become again rare and idiosyncratic situations. Rescue from market withdrawal by capability to identify target groups can often result. Less expensive testing of combinations of drugs and more information on the mechanisms of drug adverse events will result in better understanding of how different individuals respond to particular treatment regimens.

### IX. Computer Systems

Computer systems are important in being able to handle and analyze the enormous amounts of information, and to process and summarize the results. The present invention begins with the means and strategy to identify the likely candidates for large scale genome evaluation. By narrowing the search from some 30K human genes down to a small fraction (0.2-2%) for defined organ toxicity and/or mechanisms, the task of looking for appropriate features corresponding to those markers is dramatically decreased. The computer means to do the correlation have been described in detail, here and elsewhere. Many textbooks and the patent literature describe those in some detail.

Specific forms of toxicity are approached, looking for targets or pathways relevant to specific mechanisms focused, e.g., on liver, muscle, neurologic, bone marrow, gastrointestinal, kidney, skin, immune system, etc. Within each of these mechanisms, the relevant genetic markers are identified. Storing these data into files on a computer then can direct where and how to look for each type of toxicity. With a catalog of the different forms and mechanisms of toxicity, and a map of the relevant classifier biomarkers, the system then provides enormous power to catalog the modes of where to look within the organismic system for the earmarks of the toxicity pathway from initiation, progression, and outcome.

Computer systems applicable to biological applications are described, e.g., in Skierczynski and Schonk U.S. Pat. No. 6,934,636; Nahum and Stanislaw U.S. Pat. No. 6,695,780; Otvos U.S. Pat. No. 6,653,140; Singh U.S. Pat. No. 6,560,541; Stults, et al. US Pat App 20060027744; Hall and Gordon US Pat App 20040253215; and Heller, et al. US Pat App 20040236603, each incorporated herein by reference. Particularly relevant are computer systems which can evaluate features which allow subsetting patients into various risk groups, e.g., using means to determine personal risk with various therapeutic alternatives. Often some features may include medical record data, genetic data, historic medical information, and additional diagnostic features.

Computer systems will incorporate or utilize the files which catalog and link forms or pathways of toxicity, e.g., with data underlying the classifier biomarkers. Scanning through the classifier biomarker sets, common biomarkers which can indicate toxicity in various organs or locations can be identified, leading to selection of features or parameters which can evaluate the status of toxicity pathways across a wide range of locations of biological samples. Samples from different organs or locations may be evaluated on a common evaluation platform to simplify testing.

Other circumstances may require continuous monitoring of particular features. Dynamic patterns of features may show earmarks of lack of toxic effect, initiation, progression, and unavoidable toxic response. Dynamic monitoring may allow identification of when symptoms will become serious, and when certain therapeutic interventions must be substituted or changed as progression approaches irreversibility. Alternatively, pathway progression may be blocked by therapeutic intervention, e.g., with another approved drug, or known intervention (diet, other treatment). Computer systems to identify what to evaluate and when will either contain files which point out critical correlations, or are based on programs inherently using such information.

Thus, the invention provides files which identify or list relevant biomarkers linked to the specific toxicity mechanisms studied. These files will be incorporate into computer systems, directly or indirectly, through software. The patterns of genotypes, gene expression, protein expression, protein modification, post-translational modification, RNA features, and the like will be contained in similar files.

### X. Business Methods

With identified classifier biomarkers, whether based on SNPs, other genetic elements, or other features of expression or function, there should be commercial opportunities for diagnostics based thereon. Diagnostic products, services, and related commercial opportunities will result when the underlying genetic or physiological bases of toxicity are understood. Knowing where, when, and how to look can tell who may experience various categories of risk. Specific testing may subset target patients into those who are more or less likely to respond negatively to a particular therapy or drug regimen.

Also recognizing appropriate markers indicative of toxicity mechanisms will allow use in therapeutic or drug development efforts, e.g., as diagnostic complements to subset patients by efficacy, risk, or toxicity pathway activation. Monitoring over time will allow recognition of the features where the toxicity pathway will erupt. Test systems evaluating identified markers which exhibit high correlation to safety in intact systems may be useful to test compounds early in development efforts. Prioritizing compounds in development before they are administered to humans will allow upgrading of candidates early, and accurate toxicity evaluation long before expensive clinical testing is reached. Moreover, test systems which approximate disease states will result, leading to better predictive systems for both toxicity testing in the context of background disease and test platforms to determine response to relatively rarely occurring clinical situation, e.g., combinations of treatments or response to combination drug therapies.

Understanding mechanisms and/or pathways of toxicity initiation or progression present the possibility of intervening to block the response. Particular therapeutic means to block ADR progression, e.g., by increasing a clearance mechanism, inducing a bypass mechanisms to shunt toxic entities (whether primary compound or metabolically toxified) or blocking a secondary target, may be identified. Alternatively, monitoring the progression in a patient may allow safer continuing administration before termination, e.g., allowing less frequent substitution of alternative therapy or the like.

In addition, better understanding of the pathways of toxicity will provide better insights into the pathways of disease. Similar strategies to understand toxicity pathways will be applicable to understand disease pathways. Methodologies can be developed to analyze toxicity or other pathways, combining (1) the genetic correlation to combinations of diploid haplotype or allelic biomarkers, often in collections of classifier biomarkers; (2) systems biology understanding of the pathways and alternative entities to bypass or continue physiological functions, and recognizing where in the organism (which organs) and the features of biomarkers to evaluate; (3) evaluating dynamic patterns which will be useful to identify earmarks of absence, initiation, progression, or past status of the pathway; and (4) using homogeneous genetic populations with medical records or large sample banks allowing selection of phenotypically homogeneous collections for analysis.

Dosing regimens, or combination drug dosings, may be evaluated or monitored. Threshold toxic levels of combination treatments can be established, monitored, or identified, allowing combination therapies to affect a common target, but having sub-threshold negative effects. Timing aspects of pharmacology may be much better defined and carefully monitored individually, as relevant to specific patients.

Computer simulation may allow prediction of toxicity response in humans, as computer models today allow aerodynamic design formerly requiring wind tunnel tests.

While much of the discussion herein refers to human therapeutic targets, the same applications will be easily used in the context of veterinary treatment. Thus, the methods will not be limited to human analyses, but will be applicable to other groups, e.g., mammals, primates, species typically used in clinical testing, e.g., rats, mice, dogs, cats, chimpanzees and other primates and subprimates; to various types of animal functions, e.g., companion (dogs, cats, rabbits, etc.), food (birds, goats, sheep, cows, pigs, snakes, etc.), work (elephants, camels, ox, llamas, horses, dogs, etc.), and show animals (horses, aquatic animals, etc.); to structural categories, e.g., quadrupeds, bipeds, flying animals, aquatic animals; to particular subsets of species including standard experimental species from fungi (including neurospora, yeast), prokaryotes, protozoa (e.g., malaria, trypanosomes, etc.), in plants, insects (flies, water flea, pests), worms (nematodes, segmented or otherwise), invertebrates or vertebrates, and other creatures. In particular, some applications of the invention to “pests” might be to find more toxic substances which have little or no effect on other species.

In particular, the present invention is directed to various methods, both for analyses and for diagnosis. It is intended that methods where one or more steps are performed outside of the jurisdiction of a country where information is gathered, analyzed, used, or treatment decisions are made. For this reason, methods where the information is communicated to persons within a legal jurisdiction are described, including where the persons are a patient, health care professional (human or veterinary), health care insurer or auditor, or drug marketing or regulatory agency. The information may be transmitted, e.g., in written, oral, or coded forms, or in analog, digital, or encrypted forms.

In addition, devices designed for use in these methods are also encompassed by the invention. Thus, the cell lines, systems, and the like used in these analyses are incorporated; as are kits and diagnostic systems used in manual, automated, robotic, systems. Preferably, the systems will provide results rapidly, reproducibly, and with minimal manual handling, e.g., which will minimize variability and promote diagnostic validation.

Having now generally described the invention, the same will be more readily understood through reference to the following examples which are provided by way of illustration, and are not intended to be limiting of the present invention, unless specified.

### EXAMPLES

### I. General Methods

Methods for genetic analysis are well known, especially in the era of microarray analysis of genes. Specific evaluation of the full intact sequence of alleles is also common, which may include full sequencing, hybridization to selected probes, PCR analysis, and others. General methods of molecular biology are well known. See, e.g., Ausubel (ed.), et al. (2002) Short Protocols in Molecular Biology (Short Protocols in Molecular Biology; 5th ed.) Current Protocols, ISBN: 0471250929); Sambrook, et al. (2001) Molecular Cloning: A Laboratory Manual (vol. 1-3) CSH Lab. Pr. and affiliated www.MolecularCloning.com site that is evolving into an on-line laboratory manual; Cutler (ed. 2004) Protein Purification Protocols (Methods in Molecular Biology; 2d ed.) Humana Press, ISBN: 1588290670; Coligan, et al. (2001) Current Protocols in Protein Science Wiley, ISBN: 0471356808; Dickson and Mendenhall (eds. 2004) Signal Transduction Protocols (Methods in Molecular Biology; 2d ed.) Humana Press, ISBN: 1588292452; Waldman (ed. 2004) Genetic Recombination: Reviews and Protocols (Methods in Molecular Biology) Humana Press, ISBN: 1588292363; Schneider (ed. 2000) Chaperonin Protocols (Methods in Molecular Biology) Humana Press, ISBN: 0896037398; van de Heuvel (ed. 1997) PCR Protocols in Molecular Toxicology CRC-Press, ISBN: 084933344X; Fan (ed. 2002) Molecular Cytogenetics: Protocols and Applications (Methods in Molecular Biology) Humana Press, ISBN: 1588290069; Selinsky (ed. 2003) Membrane Protein Protocols: Expression, Purification, and Characterization (Methods in Molecular Biology) Humana Press, ISBN: 1588291243; Theophilus, et al. (2002) PCR Mutation Detection Protocols: Methods in Molecular Biology (Methods in Molecular Biology) Humana Press, ISBN: 0896036170; Wise (ed. 2002) Epithelial Cell Culture Protocols (Methods in Molecular Biology) Humana Press, ISBN: 0896038939; Brownstein and Khodursky (eds. 2003) Functional Genomics: Methods and Protocols (Methods in Molecular Biology) Humana Press, ISBN: 1588292916; Aguilar (ed. 2004) HPLC of Peptides and Proteins: Methods and Protocols (Methods in Molecular Biology) Humana Press, ISBN: 0896039773; Helfrich and Ralson (eds. 2003) Bone Research Protocols (Methods in Molecular Medicine) Humana Press, ISBN: 1588290441; Janzen (ed. 2002) High Throughput Screening: Methods and Protocols (Methods in Molecular Biology) Humana Press, ISBN: 0896038890; Killeen (ed. 2001) Molecular Pathology Protocols Humana Press, ISBN: 0896036812; Sioud (ed. 2004) Ribozymes and siRNA Protocols (Methods in Molecular Biology; 2d ed.) Humana Press, ISBN: 1588292266; and other volumes in the Humana Press Methods in Molecular Biology/Molecular Medicine series (see www.humanapress.com or BioMedProtocols.com); in the Methods in Enzymology series; in the periodical “Nature Methods”; or the like.

### II. Collection of Samples (Animal/Human)

Biological samples are collected from appropriate subjects, e.g., animal or human. These may be human patients, e.g., persons exhibiting a phenotype, e.g., unfavorable drug effects. Conversely, the persons may be identified as persons experiencing no unfavorable drug effects, i.e., are low risk patients. Subsetting of patients into the classifications of unfavorable or lack of unfavorable drug effects will be useful, and the statistical analysis typically requires both in blinded analyses. Collection of associated medical data or the like is very useful, e.g., including behavioral, life style, and associated medical, disease, or treatment information. Samples are often banked as part of a clinical trial, and associated medical records can be of great annotation value.

Experimental animal subjects may be preferred for certain studies, as many fewer limitations exist for sampling. Animal sampling typically can be both more invasive, generally not limited as to type or amount, and will generally be less expensive. Human studies have limitations provided both by ethical (type, amount, purpose, consent) and economic concerns.

The samplings may include one or more types, e.g., liquid, cellular, serum, tissue, hair, skin, fluid, etc., materials to evaluate genetics, expression, metabolism, or the like, of appropriate biomarkers. Samples will preferably be immediately evaluated, or may be preserved after appropriate treatment for later analysis, e.g., freezing, fixation, or other preservation methods, consistent with the type of analysis to be applied. Animal samples may be easier to evaluate, archive, and evaluate for different parameters at a later date.

Preferably the target or sample population will be a homogeneous population, exhibiting low genetic diversity and minimal introduction of genetic diversity from outsiders. Statistical concerns should be recognized, so the statistical power of the study can provide useful conclusions. Genetic analysis of small numbers of individuals in such a population will point to specific biomarkers which will suggest pathways likely to be implicated in phenotype, e.g., therapeutic outcomes. But the correlations may be weak and indistinguishable from noise. Thus, large homogeneous populations linked to medical records and related data are particularly useful, e.g., the Icelandic or a similar population. Alternatively, selection of banked samples may be based upon similarity in phenotype or genotype.

Alternatively, animal studies may be used, which will be useful in identifying gene markers correlating to particular phenotypes. The relevance of animal studies to human phenotypes is a consideration in study design.

Datasets are accessible evaluating toxicity in various organs. The first is liver toxicity, studied in rat, mouse, and dog. Other organ toxicities of interest include muscle toxicity (fatigue, pain, and cardiovascular muscle problems), CNS, bone marrow (immune system and other effects), GI tract (which similarly has rapid cell replication), kidney (clearance function), skin (fast replication), and lung (enormous surface area).

### III: Sample Evaluation

Many diagnostic methods are useful for evaluating biological samples, preferably using Good Laboratory Practices. See, e.g., Nicoll, et al (2003) Pocket Guide to Diagnostic Tests (LANGE Clinical Science, 4th ed.) McGraw-Hill Medical, ISBN: 0071411844; Gallin (2002) Principles and Practice of Clinical Research Academic Press, ISBN: 0122740653; Daniels (2002) Delmar's Manual of Laboratory and Diagnostic Tests Thomson Delmar Learning, ISBN: 0766862356; Anderson (2002) GLP Essentials: A Concise Guide to Good Laboratory Practice (2d Ed.) CRC Press, ISBN: 1574911384; Weinberg (2002) Good Laboratory Practice Regulations (Drugs and the Pharmaceutical Sciences: a Series of Textbooks and Monographs, 3d ed.) Marcel Dekker, ISBN: 0824708911; Springhouse (2001) Clinical Laboratory Tests: Values and Implications (3d Ed.) Lippincott Williams and Wilkins, ISBN: 1582550816; Abraham, et al. (2004) Trends in Biotechnology 22:15-22; and Frank and Hargreaves (2003) Drug Discovery 2:566-580.

High density microarray evaluation of genes is quite attractive, e.g., using Affymetrix or similar technologies. See, e.g., Baldi, et al. (2002) DNA Microarrays and Gene Expression: From Experiments to Data Analysis and Modeling Cambridge University Press, ISBN: 0521800226; Simon, et al (2004) Design and Analysis of DNA Microarray Investigations Springer, ISBN: 0387001352; Knudsen (2004) Guide to Analysis of DNA Microarray Data (2d ed.) Wiley-Liss, ISBN: 0471656046; Speed (ed. 2003) Statistical Analysis of Gene Expression Microarray Data Chapman and Hall/CRC, ISBN: 1584883278; Draghici (2003) Data Analysis Tools for DNA Microarrays (Bk and CD-ROM) Chapman and Hall/CRC, ISBN: 1584883154; Schena (2002) Microarray Analysis Wiley-Liss, ISBN: 0471414433; and others. Additionally, lower density arrays or individual bead or PCR analyses platforms may be used.

In one embodiment of the invention, once genes of interest have been identified, PCR analysis is performed to determine the “universe” of corresponding alleles in the human population. The region of the alleles can be localized to relatively short segments of chromosomal sequence, perhaps some “few” kb in length. Alternatively, RNA analysis may also be performed with the introns spliced out and evaluate RNA sequences. Identifying the specific alleles being expressed (among a known universe of possibilities) may also include “PCR type” amplification steps to reduce background noise. Appropriate primer are selected and used to address the relevant region of the genome. That region would be amplified, and the other portions of the genome fall out (reducing background noise). For example, selected primers may vary among up to 10-15 different specific allele sequences. Numerous dyes are available to determine which pairs out of the possibilities have been used (e.g., current FACS systems can distinguish over 10-15 different fluorescent wavelengths. Primers which hybridize to each of the polymorphisms, we can incorporate (or hybridize) differently labeled primers to determine the 15 different primers which have been incorporated. The presence of two different alleles forming a diploid pair is confirmed, for example by assigning one set of primers to one allele and another set of primers to the other allele (e.g., primers (by wavelengths) 2, 6 and 14 would assigned to one allele, while 1, 4 and 14 would be assigned to a second, and so on). Such “minisequencing” is described for example in Liljedahl, et al. (2004) BMC Biotechnology 2:24 (doi:10.1186/1472-6750-4-24); Shi (2001) “Enabling large-scale pharmacogenetic studies by high-throughput mutation detection and genotyping technologies” Clin. Chem. 47:164-72.

Exemplary datasets directed to similar problems have been performed, e.g., in Boess, et al. US Pat. App. 20040005547 “Biomarkers and expression profiles for toxicology”; Durham, et al. “Identification of biomarkers for liver toxicity” US Pat. App. 20040265889; Gut, et al. “Individual drug safety” US Pat. App. 20050037366.

Other methodologies may be applied, and may involve analyses which span multiple methodologies. See, e.g., Evans (2004) A Handbook of Bioanalysis and Drug Metabolism CRC Press, ISBN: 0415275199; Matson (2004) Applying Genomic and Proteomic Microarray Technology in Drug Discovery CRC Press; ISBN: 0849314690; and Albala and Humphery-Smith (2003) Protein Arrays, Biochips, and Proteomics Marcel Dekker, ISBN: 0824743121. Many techniques of analyses may be applied to proteomics or metabolomics, as described above.

Genetic analyses may include analyses of, e.g., quantitative DNA levels (genetic copy number; genetic duplication; genetic deletion; etc.), qualitative DNA features (polymorphisms; mutations; variations; regulatory features; and other features of structural or regulatory components), and other structural or functional DNA features (methylation, acetylation, other modifications or features). See, e.g., Fuchs and Podda (2004) Encyclopedia of Medical Genomics and Proteomics (2 vols.), Marcel Dekker, ISBN: 0824755618; Redei (2003) Encyclopedic Dictionary of Genetics, Genomics, and Proteomics (2d ed.), Wiley-Liss, ISBN: 0471268216; and Campbell and Heyer (2002) Discovering Genomics, Proteomics, and Bioinformatics (Bk and CD-Rom ed.) Benjamin Cummings, ISBN: 0805347224.

Haplotype analyses include, e.g., complete haplotype analyses in each individual (considering the entire complement of possible or related haplotypes or alleles exhibited across a population, including functionally related or other variant forms), analyses of genetic copy number and expression regulatory differences (gene duplications, gene amplification analyses), and particularly how specific haplotypes interact with other combinations of haplotypes or related haplotypes which affect biological function of a particular genotype. For example a “dominant” haplotype may be recessive to multiple copies of a “recessive” haplotype, and certain forms of gene dosage effects may depend upon gene copy numbers (e.g., in chromosome duplications) or regulatory segments controlling an allele. In particular, the phenotypic result from peculiar combinations of alleles may not comport with the simplistic Mendelian model of an inherent “dominance” or “recessiveness” of specific alleles. And in circumstances where kinetics are critical, e.g., in metabolic pathways, the flux of reactants may be affected by the reaction or turnover rates of the relevant (source/sink) enzymes, and the ultimate accumulation of particular reactants may depend upon the relative expression levels or turnover numbers of the respective producing or reacting enzymes. In many cases, problems in the turnover number of a particular expressed allele may be compensated by over/under expression of a different upstream or downstream enzymatic function, by activity of a modulating effector, or by the compensating expression of a different allele in the upstream or downstream functionality. Moreover, the presence of a different related (e.g., overlapping) enzymatic function may affect the impact of a specific allele. Thus, correlation with the whole spectrum of related functions will be valuable in understanding that simple genetic correlation to individual alleles will often fail to provide optimum correlation, and may often cause sufficiently poor correlation to effectively assist in therapeutic decision making.

Expression analyses, typically related to mRNA molecules, include, e.g., expression regulation (transcription; mRNA turnover; mRNA lifetime; translation effectors; and splice variants (many of which may exhibit different phenotypic function)). PCR or related methodologies may be used to qualitatively define and quantitate specific allelic forms.

Evaluation of proteins, e.g., by proteomic analyses, will often distinguish between forms which can exhibit different phenotypes, e.g., functional differences. Variants in sequence, amount, modification (glycosylation, phosphorylation, etc.), cofactors, subunit interactions (especially in multiprotein complexes), and such can result in differences in activity, function, or other phenotype.

Many methodologies to evaluate functional biological entities have been described, e.g., Walker (2005) Proteomics Protocols Handbook Humana Press, ISBN: 1588293432; Hamdan and Righetti (2005) Proteomics Today: Protein Assessment and Biomarkers Using Mass Spectrometry, 2D Electrophoresis, and Microarray Technology (Wiley-Interscience Series on Mass Spectrometry), Wiley-Interscience, ISBN: 0471648175; Simpson (2004) Purifying Proteins for Proteomics: A Laboratory Manual CSH Laboratory Press, ISBN: 0879696966; Cheng and Hammar (eds. 2004) Conformational Proteomics of Macromolecular Architecture: Approaching the Structure of Large Molecular Assemblies and Their Mechanisms of Action (with CD-Rom), World Sci. Pub., ISBN: 9812386157; Sanchez (ed. 2004) Biomedical Applications of Proteomics Wiley, ISBN: 3527308075; Twyman (2004) Principles Of Proteomics (Advanced Text Series), BIOS Sci. Pub., ISBN: 1859962734; Conn (2003) Handbook of Proteomic Methods Humana Press, ISBN: 1588293408; Westermeier, et al (2002) Proteomics in Practice: A Laboratory Manual of Proteome Analysis Wiley-VCH, ISBN: 3527303545; Simpson (2002) Proteins and Proteomics: A Laboratory Manual CSH Laboratory Press, ISBN: 0879695544; and Liebler (2001) Introduction to Proteomics: Tools for the New Biology Humana Press, ISBN: 0896039919.

For example, certain modifications (methylation, glycosylation, phosphorylation, acetylation, ubiquitination, and many others) to a protein may affect enzymatic turnover number, biological activity, half-life, turnover, substrate or other (e.g., regulatory) specificity or selectivity, temperature or other environmental sensitivity, enhancer or suppressor efficiency, and many other features which affect ultimate biological function in a particular context. See, e.g., Lee, et al. (2003) Drug Metabolism Enzymes Marcel Dekker, ISBN: 0824742931; and Testa and Mayer (2003) Hydrolysis in Drug and Prodrug Metabolism: Chemistry, Biochemistry, and Enzymology Wiley-VCH, ISBN: 390639025X. Chaparone proteins and protein conformation or folding scaffolds may be important, and often the functional unit will be other than specific genes or gene products. For example, ribosome function probably is dramatically affected by the many protein and RNA components which contribute to its structure.

In situ hybridization methods may evaluate the fidelity of organelle or cellular compartment localization, trafficking integrity and efficiency, and other gross biological functions which may sensitively distinguish or mediate various allelic differences.

Other methodologies exist, which may be direct or indirect, which may be used as surrogate means to evaluate particular features of the biological functions, e.g., PET scans, Gas Chromatography, other non-invasive or invasive spectroscopy or other methods which may be important to describe, which may be used to evaluate biochemical features which could be used herein. See, e.g., Korfmacher (2005) Using Mass Spectrometry for Drug Metabolism Studies CRC Press, ISBN: 0849319633; and Pfleger, et al (2000) Mass Spectral and GC Data of Drugs, Poisons, Pesticides, Pollutants and Their Metabolites (2d Ed.) Wiley-VCH, ISBN: 3527288805.

Potentially, accessible samples will often be most amenable to genetic analyses since samples may exist historically or in archival forms. Blood samples are reasonably common and easily collected. Typically DNA and related methodologies, e.g., PCR, hybridization, sequencing, restriction analyses, and the like, are usually “qualitative”, reasonably sensitive, and reasonably unambiguous in analysis. Moreover, sample handling tends to be simple and reproducible. Samples may be collected by non-invasive methods, e.g., hair/fingernail/skin/mucosal samples. Other non-invasive techniques such as X-ray, MRI, or related imaging methods; stool/urine/saliva/mucous samples; reproductive fluids; tears; exhalation; and external analytical methods may be used.

### IV: Evaluation Interpretation

Statistical correlation analysis of phenotype with analyses will identify, in rank order, those markers exhibiting statistical correlation. Extending the analyses to correlate to haplotype combinations can also be performed where specific haplotypes or alleles are each evaluated. Simplistically, assuming haplotypes or alleles are only pairwise (e.g., diploid only), correlations with specific pairs can be evaluated for phenotype. Extending this further, correlations should include alternative combinations, including situation where one chromosome (or part thereof) may be duplicated, where gene dosage or dramatic regulatory effects may be evaluated, or where functionally equivalent alternative genetic sites may affect penetrance to phenotype.

In many situations, the haplotypes or markers will obviously indicate specific metabolic or enzymatic pathways or networks which correlate to phenotype. Alternatively, various pathways will emerge as being critical, and the members of the pathways or networks can be evaluated more closely.

In the analysis, certain patterns will be identified which account for most of the genetic variations contained in the target population (e.g., experiencing the particular effect). For example, in the context of genetic allele analysis for many genes, preferably the entire structural genome, the presence or absence of allele pairings is evaluated. The evaluation may take many forms, but the principal forms include Principal Component Analysis (PCA), various clustering techniques, supervised clustering techniques, and other statistical methods referred to above. These data will provide information which can be combined with systems biology and genomic cross species correlations to understand what networks and what members of these networks are likely targets to be useful signature factors. These factors will be those which are directly correlated to the features, typically combinations, which together define the phenotype.

### A. Boess Liver Toxicity Dataset

Applying the Gene Ontology software on rat liver toxicity markers from Boess, et al. US Pat. App. 20040005547 “Biomarkers and expression profiles for toxicology” provides the results of Table 1.

Table 2A lists gene ID numbers for primate counterpart biomarker genes derived from the dataset from the Boess, et al. patent. Human and chimpanzee counterparts are identified, and other species can be similarly listed where sufficient information is available on the genome. The human subset 1 provides counterpart human genes, expressly listing the Entrez ID, the accepted symbol, and a short description of the gene corresponding to the marker. See http://www.ncbi.nlm.nih.gov/entrez/query.fcgi?db=gene, which database is described, e.g., in Maglott, et al. (2005) Nucl. Acids Res. 33: database issue doi:10.1093/nar/gkiO31. The human subset 2 are Entrez gene IDs of genes which are reported to interact directly (e.g., by physical association or 2-hybrid interaction) with markers of subset 1, either reported from human or other species counterpart. The human subset 3 are Entrez gene IDs of markers which have been associated by being referred to in a published abstract with one of the markers of subset 1.

Similarly, chimpanzee counterparts are listed by Entrez gene ID numbers.

Table 2B lists Entrez gene ID numbers for counterparts in selected non-primate species. These are provided in dog, rat, and mouse. Similar counterparts can be generated for other species, as the genome sequences of additional species become more complete and counterpart equivalents can be determined. Typically the counterparts are assigned by sequence relatedness, genetic location in closely related species, or functional equivalence.

TABLE 1
Gene Ontology analysis:

| Biological process: | | | |
|---|---|---|---|
| GO: 0000003 | reproduction | Hmgcr Clu | 0.54 |
| GO: 0007154 | cell communication | Erbb3 Ppp5c Glul Hrmt1l2 Ccl2 Socs2 Pik3r1 Apob Enpp2 Pde2a | 24.59 |
| | | Apoe Ripk3 Lamr1 Hspca Pdk1 Nqo1 Npr1 Arl5 Itga7 Igfbp1 Igfbp3 | |
| | | Gja5 App Srd5a1 Srd5a2 Pld2 Nxf Ptpn1 Slc1a1 Il1a Cdh17 Gjb5 | |
| | | Reln Sqstm1 Mme Icam1 Tieg Gcgr Il9 Scarb2 Atp2a2 Ptpns1 Gucy1b2 | |
| | | Ms4a2 Abat Il4r Adora2b Grp Nmu Gpr48 Gria1 Aplp2 Pak2 Tnfrsf1a | |
| | | Tnfrsf1b Slc8a2 Crem Ica1 Rab38 Cdc42bpa Cfl1 Stat3 Ahr Selp Gfra3 | |
| | | Igf1 Dusp6 Psen2 Nr4a2 Grin2d Adam17 Ngfr Kcnh2 Agtr1 Arf4 | |
| | | Txnrd1 Grin3b Kcnmb1 Mapkapk2 Nfkbia Trib3 Cxcl1 Pik3c2g Igfals | |
| | | Cxcl10 Rara Pex11a Kng1 Tdo2 Hmox1 Avpr1a | |
| GO: 0007389 | pattern specification | Pitx2 | 0.27 |
| GO: 0007530 | sex determination | Srd5a1 Srd5a2 | 0.54 |
| GO: 0007548 | sex differentiation | Srd5a1 Srd5a2 Cyp17a1 | 0.81 |
| GO: 0007568 | aging | Tp53 Npm1 | 0.54 |
| GO: 0007611 | learning and/or memory | Apoe | 0.27 |
| GO: 0007622 | rhythmic behavior | Dbp | 0.27 |
| GO: 0007632 | visual behavior | Dncl2a | 0.27 |
| GO: 0008152 | metabolism | Pfkfb1 Gsto1 Hpcl2 Sqle Egr1 Agxt Aldh6a1 Erbb3 Hnrpa1 Ppp5c | 70.54 |
| | | Nr0b2 Thrsp Ptp4a1 Glul Minpp1 Man2b1 Hrmt1l2 Pdhb Ppp1r2 Por | |
| | | Ccl2 Fabp5 Ebp G10 Oazin Eif4ebp1 Gsta5 Pygl Supt5h Apob | |
| | | Enpp2 Enpp3 Ca5a Apoe Ripk3 Myc Sult1a1 Sult1a2 Lamr1 Timp1 | |
| | | Apoa1 Apoa2 Eno1 Hmgcr Hspca Sfpq Pdk1 Rpl13 Ttr Otc Nqo1 Npr1 | |
| | | Atf3 Sult1b1 Ltb4dh Sc4mol Cyp4f2 Ap1b1 Dpp4 Phkg2 Srd5a2 | |
| | | Rpl22 Nap1l1 Ppil3 Rpl28 Fdft1 Pld2 Odc1 G6pc Ptpn1 Cbr1 Pcna | |
| | | Apoc1 Apoc3 Serpinh1 Jun Cyp1a1 Sqstm1 Rpl3 Mme Rpl6 Zfp36 | |
| | | Padi3 Rpl13a Fmo1 Lpl Tpp2 Idi1 Clu Phgdh Nr1d1 Tieg Gcgr Dkc1 | |
| | | Insig1 Ada Nfe2l2 Prss2 Gamt Fasn Nat1 Asl Alpl Tomm20 Atp2a2 | |
| | | Psma1 Tp53 Psma5 Acly Psma7 Bteb1 Casp3 Got1 Akr7a3 Cdc37 Pax4 | |
| | | Junb Cyp7a1 Atp6v1b2 Eif2b1 Gucy1b2 Eif2b3 Pitx2 Atic Ca3 Psmb1 | |
| | | Psmb3 Psmb4 Psmb5 Ugt1a6 Abat Adora2b Gck Aldoa Sfrs5 Cyp7b1 | |
| | | Pnpo Ddit3 Psmc2 Phb Psmc3 Gapd Pak2 Gsr Sgta Copeb Gss Eif5 | |
| | | Bdh Eif2s1 Crem Cat Gtf2f2 Smc1l1 Cpz Dnajb9 Cdc42bpa Onecut1 | |
| | | Btg2 Gusb Cong1 Fads1 Srebf1 Decr2 Kynu Slc3a2 Bzrp Pkm2 Asns | |
| | | Cyb5 Stat3 Abcd2 Acsl3 Lrp2 Foxa2 Ptbp1 Ssr3 Baat Ahr Acox2 Amd1 | |
| | | Apobec1 Pklr Sardh Csda Limk2 Dusp1 Igf1 Apex1 Cd74 Dusp5 Dusp6 | |
| | | Psen2 Cyba Vhl Cyp17a1 Nfia Htatip Nfib Nr4a2 Gtf2ird1 Adam17 | |
| | | Cyp2e1 Cdkn1a Gm2a Gadd45a Cebpb Rps2 Kcnh2 Hao2 Cdipt Rps5 | |
| | | Maob Fdps Camkk1 Bach Calr Txnrd1 Mybbp1a Eef2 Smpd3 Prdx6 | |
| | | Mapkapk2 Bhmt Id1 Npm1 Hmgcs1 Rnase4 Cth Irf3 Gstm1 Cel Dia1 | |
| | | Dbp Nfkbia Trib3 Mat1a Ggps1 H1f0 Gpx1 Tat Rara Sds Prss15 Ctsl | |
| | | SMN1 Cited2 Pmpca Ehhadh Pmpcb Oat Hspa8 Znf354a Aldh2 Dpys | |
| | | Anpep Tdo2 Hmox1 Pck1 Sod1 Avpr1a | |
| GO: 0009653 | morphogenesis | Ccl2 Fabp5 Socs2 Ebp Apoe Csrp2 Hmgcr Cspg5 Ifrd1 Npr1 Itga7 | 12.16 |
| | | Igfbp1 Igfbp3 Srd5a2 Gjb5 Phgdh Tieg Alpl Col1a1 Atp2a2 Tp53 Pax4 | |
| | | Pitx2 Vil2 Copeb Gss Slc3a2 Stat3 Lmna Gfra3 Igf1 Vhl Apaf1 Palm | |
| | | Ngfr Col3a1 Drpla Ghr Myh3 Cxcl1 Cxcl10 Dcc Dcn Anpep Sod1 | |
| GO: 0016265 | death | Ccl2 Apoe Ripk3 Igfbp3 App Il1a Clu Bad Tp53 Casp3 Tnfrsf1a | 7.30 |
| | | Tnfrsf1b Tubb5 Ahr Psen2 Vhl Cdkn1a Apaf1 Gadd45a Ngfr Bcl2l1 | |
| | | Npm1 Nfkbia Trib3 Dcc Kng1 Alb | |
| GO: 0019058 | viral infectious cycle | Ccl2 Nfia | 0.54 |
| GO: 0030154 | cell differentiation | Socs2 Csrp2 Ifrd1 Igfbp3 Tp53 Pax4 Copeb Vhl Ndrg2 | 2.43 |
| GO: 0040007 | growth | Socs2 Csrp2 Npr1 Igfbp1 Igfbp3 Tp53 Slc3a2 Ghr | 2.16 |
| GO: 0042592 | homeostasis | Ccl2 Apoe Myc App Slc12a4 Tf Alb | 1.89 |
| GO: 0043062 | extracellular structure organization and biogenesis | Apoe | 0.27 |
| GO: 0046903 | secretion | Pigr Npr1 Ica1 Agtr1 Cel | 1.35 |
| GO: 0048066 | pigmentation | Hmox1 | 0.27 |
| GO: 0050790 | regulation of enzyme activity | Tp53 Cdc37 Adora2b Pak2 Ccng1 Psen2 Cdkn1a Gadd45a Trib3 | 2.43 |
| GO: 0050791 | regulation of physiological process | Egr1 Nr0b2 Thrsp G10 Eif4ebp1 Supt5h Myc Lamr1 Timp1 Apoa2 | 17.03 |
| | | Hspca Npr1 Atf3 Pcna Jun Sqstm1 Rpl6 Nr1d1 Tieg Gcgr Nfe2l2 | |
| | | Slc12a4 Tp53 Bteb1 Pax4 Junb Pitx2 Ddit3 Fga Phb Copeb Eif5 | |
| | | Eif2s1 Crem Ica1 Gtf2f2 Onecut1 Btg2 Srebf1 Stat3 Foxa2 Ahr | |
| | | Csda Vhl Nfia Htatip Nfib Nr4a2 Gtf2ird1 Cebpb Kcnh2 Calr | |
| | | Mybbp1a Bhmt Id1 Npm1 Irf3 Dbp Trib3 Rara Kng1 Cited2 Znf354a | |
| GO: 0050793 | regulation of development | Socs2 Npr1 Igfbp1 Igfbp3 Tp53 Vhl | 1.62 |
| GO: 0050794 | regulation of cellular process | Ppp5c Ccl2 Socs2 Apoe Ripk3 Timp1 Csrp2 Npr1 Igfbp1 Igfbp3 | 11.08 |
| | | Nap1l1 Il1a Sqstm1 Tieg Il9 Bad Slc12a4 Tp53 Casp3 Ap2s1 Fga | |
| | | Phb Trpv5 Tnfrsf1a Btg2 Btg3 Slc3a2 Igf1 Vhl Cdkn1a Apaf1 Palm | |
| | | Bcl2l1 Npm1 Nfkbia Cxcl1 Cxcl10 Dcc Kng1 Alb Hmox1 | |
| GO: 0050817 | coagulation | Serpind1 Serpine1 Fga Kng1 | 1.08 |
| GO: 0050874 | organismal physiological process | Serpind1 Glul Ccl2 Serpine1 Apob Apoe Apoa1 Apoa2 Nqo1 Npr1 | 15.68 |
| | | Dpp4 Slc1a1 Il1a Lpl Clu Gcgr Il9 Ada Prss2 Slc12a4 Lbp Alpl | |
| | | Col1a1 Tap1 Scnn1b Ms4a2 Abat Il4r Adora2b Nmu Gria1 Fga Copeb | |
| | | Abcc6 Ica1 Stat3 Baat Cd74 Nr4a2 Grin2d Cebpb Kcnh2 Agtr1 | |
| | | Col3a1 Grin3b Kcnmb1 Cel C1qbp Dia1 Rbp4 Cxcl1 Cxcl10 Kng1 Alb | |
| | | Oat Znf354a Tdo2 Avpr1a | |
| GO: 0050875 | cellular physiological process | Slc23a1 Hnrpa1 Slc10a1 Ppp5c Ccl2 Fabp5 Socs2 A2m Supt5h | 40.27 |
| | | Cacna1d Apob Enpp2 Apoe Ripk3 Myc Timp1 Csrp2 Apoa1 Apoa2 | |
| | | Slco2a1 Hmgcr Hspca Cspg5 Ttr Npr1 Itga7 Ap1b1 Igfbp1 Slc6a11 | |
| | | Igfbp3 Gja5 App Nap1l1 Slc6a9 Pld2 Slc1a1 Pcna Inhbe Il1a Apoc1 | |
| | | Apoc3 Cdh17 Jun Atp1b1 Sqstm1 Kcna5 Clu Slc17a1 Tieg Dkc1 Il9 | |
| | | Insig1 Bad Slc20a2 Gamt Slc12a4 Lbp Tomm20 Col1a1 Dnch1 Atp2a2 | |
| | | Tp53 Cltb Ccnd1 Casp3 Tap1 Cdc37 Atp6v1b2 Scnn1b Vil2 Ms4a2 | |
| | | Adora2b Aldoa Abcb1 Ap2s1 Nmu Afm Gria1 Ddit3 Trpv1 Fga Phb | |
| | | Trpv5 Copeb Tnfrsf1a Tnfrsf1b Abcc6 Abcc9 Slc8a2 Hbb Ica1 | |
| | | Smc1l1 Rab38 Btg2 Btg3 Lcn2 Ccng1 Cfl1 Slc3a2 Bzrp Stat3 Abcd2 | |
| | | Abcd3 Dncl2a Lrp2 Tubb5 Ssr3 Pscd2 Ahr Dusp1 Igf1 Lim2 Dusp6 | |
| | | Psen2 Vhl Nfia Htatip Nfib Grin2d Cdkn1a Apaf1 Gadd45a Tf Palm | |
| | | Ngfr Bcl21l Kcnh2 Arf4 Col3a1 Mybbp1a Grin3b Kcnmb1 Serpina6 | |
| | | Slc4a2 Npm1 Ghr Dia1 Nfkbia Trib3 Rbp4 Myh3 Cxcl1 H1f0 Cxcl10 | |
| | | Rara Dcc Pex11a Kng1 Alb | |
| GO: 0050896 | response to stimulus | Serpind1 Hrmt1l2 Ccl2 Enpp2 Apoe Apoa2 Hspca Nqo1 Dpp4 | 17.30 |
| | | Srd5a2 Pcna Il1a Serpinh1 Sqstm1 Clu Gcgr Dkc1 Il9 Ada Lbp Col1a1 | |
| | | Tp53 Tap1 Eif2b3 Pon1 Ms4a2 Ugt1a6 Il4r Adora2b Abcb1 Ddit3 Gsr | |
| | | Copeb Gss Abcc6 Cat Smc1l1 Btg2 Stat3 Ahr Csda Dusp1 Apex1 Cd74 | |
| | | Vhl Htatip Nr4a2 Gadd45a Cebpb Kcnh2 Prdx6 Npm1 C1qbp Nfkbia | |
| | | Rbp4 Cxcl1 Gpx1 Cxcl10 Kng1 Oat Hspa8 Znf354a Dpys Sod1 | |
| GO: 0051098 | regulation of binding | Apex1 | 0.27 |
| Molecular function: | | | |
| GO: 0000146 | microfilament motor activity | Myh3 | 0.27 |
| GO: 0000155 | two-component sensor molecule activity | Kcnh2 | 0.27 |
| GO: 0000166 | nucleotide binding | Pfkfb1 Erbb3 Por Ripk3 Hspca Pdk1 Npr1 Arl5 Phkg2 Myo5b Dnch1 | 12.30 |
| | | Atp2a2 Tp53 Acly Tap1 Atp6v1b2 Eif2b1 Gck Abcb1 Psmc2 Psmc3 | |
| | | Pak2 Gss Eif5 Abcc6 Abcc9 Gtf2f2 Smc1l1 Rab38 Cdc42bpa Abcd2 | |
| | | Abcd3 Tubb5 Limk2 Apaf1 Camkk1 Arf4 Eef2 Mapkapk2 Trib3 Myh3 | |
| | | Mat1a Prss15 Hspa8 Pck1 | |
| GO: 0001871 | pattern binding | Serpind1 Apob Apoe App Rpl22 Lpl Kng1 | 1.91 |
| GO: 0003676 | nucleic acid binding | Egr1 Hnrpa1 Nr0b2 G10 Supt5h Enpp2 Enpp3 Hnrpab Myc Sfpq | 19.40 |
| | | Rpl13 Atf3 Rpl22 Rpl28 Pcna Jun Rpl3 Rpl6 Zfp36 Nr1d1 Tieg Dkc1 | |
| | | Nfe2l2 Psma1 Tp53 Bteb1 Pax4 Junb Eif2b1 Eif2b3 Pitx2 Zfp36l1 | |
| | | Sfrs5 Gpr48 Ddit3 Aplp2 Copeb Eif5 Eif2s1 Crem Gtf2f2 Smc1l1 | |
| | | Onecut1 Btg2 Srebf1 Stat3 Foxa2 Ptbp1 Ahr Apobec1 Csda Apex1 | |
| | | Nfia Nfib Nr4a2 Gtf2ird1 Cebpb Rps2 Rps5 Mybbp1a Eef2 Npm1 | |
| | | Rnase4 Irf3 Ncl Dbp H1f0 Rara SMN1 Cited2 Znf354a | |
| GO: 0003700 | transcription factor activity | Egr1 Nr0b2 G10 Supt5h Myc Atf3 Jun Nr1d1 Tieg Nfe2l2 Tp53 Bteb1 | 9.02 |
| | | Pax4 Junb Pitx2 Zfp36l1 Ddit3 Crem Onecut1 Btg2 Srebf1 Stat3 | |
| | | Foxa2 Ahr Csda Nfia Nfib Nr4a2 Gtf2ird1 Cebpb Irf3 Rara Cited2 | |
| GO: 0003702 | RNA polymerase II transcription factor activity | Jun Junb Gtf2f2 Srebf1 Csda Gtf2ird1 Irf3 Dbp Znf354a | 2.46 |
| GO: 0003711 | transcriptional elongation regulator activity | Supt5h | 0.27 |
| GO: 0003712 | transcription cofactor activity | Nr0b2 Ripk3 Atf3 Nr1d1 Junb Ddit3 Psmc3 Csda Apex1 Htatip Calr | 4.37 |
| | | Npm1 Id3 Irf3 Trib3 Rara | |
| GO: 0003735 | structural constituent of ribosome | Ptp4a1 Lamr1 Rpl13 Rpl22 Rpl28 Rpl3 Rpl6 Rpl13a Phb Rps2 Rps5 | 3.01 |
| GO: 0003777 | microtubule motor activity | Dnch1 Smc1l1 Dncl2a | 0.82 |
| GO: 0004362 | glutathione-disulfide reductase activity | Gsr | 0.27 |
| GO: 0004386 | helicase activity | Gtf2f2 | 0.27 |
| GO: 0004601 | peroxidase activity | Cat Prdx6 Gpx1 | 0.82 |
| GO: 0004791 | thioredoxin-disulfide reductase activity | Txnrd1 | 0.27 |
| GO: 0004857 | enzyme inhibitor activity | Serpind1 Ppp1r2 Oazin Serpine1 A2m Timp1 App Serpinh1 Aplp2 | 3.55 |
| | | Cdkn1a Serpina6 Trib3 Kng1 | |
| GO: 0004872 | receptor activity | Erbb3 Nr0b2 Ebp Lamr1 Npr1 Itga7 Icam1 PVR Nr1d1 Gcgr Slc20a2 | 10.38 |
| | | Scarb2 Ms4a2 Il4r Adora2b Gpr48 Gria1 Trpv1 Trpv5 Tnfrsf1a | |
| | | Tnfrsf1b Abcc9 Cpz Bzrp Lrp2 Ahr Gfra3 Cd74 Nr4a2 Grin2d | |
| | | Ngfr Agtr1 Grin3b Ghr Rara Dcc Anpep Avpr1a | |
| GO: 0005057 | receptor signaling protein activity | Il4r Stat3 | 0.55 |
| GO: 0005102 | receptor binding | Ccl2 Socs2 Apob Apoe Cspg5 Inhbe lI1a Il9 Grp Nmu Tubb5 Gfra3 | 4.37 |
| | | Igf1 Cxcl1 Cxcl10 Kng1 | |
| GO: 0005200 | structural constituent of cytoskeleton | Tubb5 Vim | 0.55 |
| GO: 0005201 | extracellular matrix structural constituent | Col1a1 Col3a1 | 0.55 |
| GO: 0005212 | structural constituent of eye lens | Lim2 | 0.27 |
| GO: 0005275 | amine transporter activity | Slc6a11 Slc6a9 Slc1a1 Slc12a4 | 1.09 |
| GO: 0005319 | lipid transporter activity | Apob Apoe Apoa1 Apoa2 Slco2a1 Apoc1 Apoc3 Lpl Cdipt | 2.46 |
| GO: 0005326 | neurotransmitter transporter activity | Slc6a11 Slc6a9 | 0.55 |
| GO: 0005342 | organic acid transporter activity | Slc10a1 Slc6a11 Slc6a9 Slc1a1 Slc12a4 | 1.37 |
| GO: 0005344 | oxygen transporter activity | Hbb | 0.27 |
| GO: 0005386 | carrier activity | Slc23a1 Slc10a1 A2m Ttr Slc6a11 Slc6a9 Slc1a1 Atp1b1 Slc17a1 | 5.46 |
| | | Slc20a2 Slc12a4 Tomm20 Atp2a2 Atp6v1b2 Afm Slc8a2 Slc3a2 Cyb5 | |
| | | Slc4a2 Alb | |
| GO: 0005489 | electron transporter activity | Por Nqo1 Srd5a1 Srd5a2 Phgdh Akr7a3 Gsr Cyba Maob Txnrd1 Dial | 3.28 |
| | | Aldh2 | |
| GO: 0005496 | steroid binding | Ttr Serpina6 | 0.55 |
| GO: 0005515 | protein binding | Cttn Nr0b2 Eif4ebp1 A2m Enpp2 Apoe Ripk3 Myc Apoa1 Apoa2 | 21.58 |
| | | Hspca Cspg5 Atf3 Itga7 Ap1b1 Igfbp1 Igfbp3 App Phkg2 Cdh17 | |
| | | Lgals3 Serpinh1 Reln Sqstm1 Kcna5 Icam1 Nr1d1 Myo5b Bad Tp53 | |
| | | Cdc37 Junb Vil2 Ms4a2 Il4r Grip2 Ddit3 Psmc3 Trpv5 Sgta | |
| | | Tnfrsf1a Tnfrsf1b Crem Smc1l1 Dnajb9 Cfl1 Lmna Dncl2a Tubb5 | |
| | | Selp Csda Limk2 Apex1 Cd74 Psen2 Vhl Htatip Apaf1 Ngfr Sdc3 | |
| | | Sdc4 Calr Vim Mybbp1a Drpla Npm1 Id3 Irf3 Nfkbia Trib3 Myh3 | |
| | | Igfals Rare Kng1 SMN1 Cited2 Pmpca Hspa8 Avpr1a | |
| GO: 0008047 | enzyme activator activity | Igfbp3 Gm2a Apaf1 Arf4 Cxcl1 | 1.37 |
| GO: 0008135 | translation factor activity, nucleic acid binding | Eif2b1 Eif2b3 Eif5 Eif2s1 Eef2 | 1.37 |
| GO: 0008144 | drug binding | Alb | 0.27 |
| GO: 0008147 | structural constituent of bone | Col1a1 | 0.27 |
| GO: 0008289 | lipid binding | Fabp5 Pik3r1 Apoe Apoa1 Apoa2 Apoc3 Lbp Nsfl1c Bach Alb | 2.73 |
| GO: 0008565 | protein transporter activity | Pigr A2m Tomm20 Ap2s1 | 1.09 |
| GO: 0009975 | cyclase activity | Npr1 Gucy1b2 | 0.55 |
| GO: 0015075 | ion transporter activity | Slc10a1 Slc6a9 Slc1a1 Atp1b1 Slc17a1 Atp2a2 Atp6v1b2 Cyb5 Slc4a2 | 2.46 |
| GO: 0015197 | peptide transporter activity | Tap1 | 0.27 |
| GO: 0015238 | drug transporter activity | Ebp | 0.27 |
| GO: 0015267 | channel or pore class transporter activity | Cacna1d Gja5 Gjb5 Kcna5 Scnn1b Ms4a2 Gria1 Trpv1 Trpv5 Grin2d | 3.55 |
| | | Kcnh2 Grin3b Kcnmb1 | |
| GO: 0015457 | auxiliary transport protein activity | Kcnmb1 | 0.27 |
| GO: 0016301 | kinase activity | Pfkfb1 Erbb3 Ccl2 Pik3r1 Ripk3 Pdk1 Npr1 Prkcdbp Phkg2 Gck Pak2 | 6.01 |
| | | Cdc42bpa Pkm2 Pklr Limk2 Cdkn1a Kcnh2 Camkk1 Mapkapk2 | |
| | | Trib3 Pik3c2g Pck1 | |
| GO: 0016491 | oxidoreductase activity | Gsto1 Sqle Aldh6a1 Pdhb Por Hmgcr Nqo1 Ltb4dh Sc4mol Cyp4f2 | 12.57 |
| | | Srd5a1 Srd5a2 Fdft1 Cbr1 Cyp1a1 Fmo1 Phgdh Fasn Akr7a3 | |
| | | Cyp7a1 Cyp7b1 Pnpo Gapd Gsr Bdh Cat Fads1 Decr2 Cyb5 | |
| | | Acox2 Sardh Apex1 Cyba Cyp17a1 Cyp2e1 Hao2 Maob Txnrd1 | |
| | | Prdx6 Dia1 Gpx1 Ehhadh Aldh2 Tdo2 Hmox1 Sod1 | |
| GO: 0016563 | transcriptional activator activity | Phb Copeb Onecut1 | 0.82 |
| GO: 0016564 | transcriptional repressor activity | Phb | 0.27 |
| GO: 0016740 | transferase activity | Pfkfb1 Gsto1 Agxt Erbb3 Hrmt1l2 Ccl2 Gsta5 Pygl Pik3r1 Ripk3 | 15.30 |
| | | Sult1a1 Sult1a2 Pdk1 Otc Npr1 Sult1b1 Phkg2 Srd5a2 | |
| | | Fdft1 Pcna Dkc1 Gamt Fasn Nat1 Acly Got1 Eif2b3 Atic | |
| | | Ugt1a6 Abat Gck Pak2 Cdc42bpa Pkm2 Acsl3 Baat Pklr | |
| | | Sardh Limk2 Htatip Cdkn1a Kcnh2 Cdipt Fdps Camkk1 | |
| | | Mybbp1a Mapkapk2 Bhmt Hmgcs1 Gstm1 Trib3 Pik3c2g Mat1a | |
| | | Ggps1 Tat Oat | |
| GO: 0016787 | hydrolase activity | Pfkfb1 Ppp5c Ptp4a1 Minpp1 Man2b1 Serpine1 Enpp2 Pde2a Enpp3 | 20.22 |
| | | Dpp4 Pld2 G6pc Ptpn1 Atp1b1 Reln Mme Padi3 Lpl Tpp2 Ada | |
| | | Prss2 Fasn Alpl Dnch1 Atp2a2 Psma1 Tp53 Psma5 Psma7 | |
| | | Casp3 Tap1 Atp6v1b2 Atic Pon1 Psmb1 Psmb3 Psmb4 Psmb5 | |
| | | Abcb1 Psmc2 Psmc3 Eif3 Abcc6 Abcc9 Smc1l1 Cpz Rab38 | |
| | | Gusb Kynu Slc3a2 Abcd2 Abcd3 Tubb5 Baat Apobec1 Dusp1 | |
| | | Apex1 Dusp5 Dusp6 Adam17 Arf4 Bach Smpd3 Prdx6 Rnase4 | |
| | | Cel Myh3 Prss15 Ctsl Pmpca Pmpcb Hspa8 Dpys Anpep | |
| GO: 0016829 | lyase activity | Hpcl2 Ca5a Eno1 Npr1 Odc1 Dkc1 Fasn Asl Gucy1b2 Ca3 Aldoa | 4.92 |
| | | Amd1 Apex1 Cth Tat Sds Ehhadh Pck1 | |
| GO: 0016853 | isomerase activity | Ebp Ppil3 Idi1 Ehhadh | 1.09 |
| GO: 0016874 | ligase activity | Glul Gss Asns Acsl3 | 1.09 |
| GO: 0019207 | kinase regulator activity | Cdkn1a Trib3 Cxcl10 | 0.82 |
| GO: 0019208 | phosphatase regulator activity | Ppp1r2 Igfbp3 | 0.55 |
| GO: 0019825 | oxygen binding | Cyp1a1 Cyp17a1 Cyp2e1 | 0.82 |
| GO: 0019840 | isoprenoid binding | Ttr Rbp4 | 0.55 |
| GO: 0019842 | vitamin binding | Ttr Abat Rbp4 Oat | 1.09 |
| GO: 0030235 | nitric-oxide synthase regulator activity | Hspca | 0.27 |
| GO: 0030246 | carbohydrate binding | Serpind1 Apob Apoe App Rpl22 Lgals3 Lpl Clecsf6 Selp Calr Kng1 | 3.01 |
| GO: 0030695 | GTPase regulator activity | Eif2b1 Cdc42bpa Pscd2 | 0.82 |
| GO: 0042165 | neurotransmitter binding | Bzrp | 0.27 |
| GO: 0042277 | peptide binding | Npr1 Ssr3 Agtr1 Avpr1a | 1.09 |
| GO: 0042562 | hormone binding | Npr1 | 0.27 |
| GO: 0043028 | caspase regulator activity | Apaf1 | 0.27 |
| GO: 0043167 | ion binding | Hpcl2 Egr1 Ppp5c Cacna1d Ca5a Csrp2 Eno1 Ltb4dh Igfbp3 Fdft1 | 13.66 |
| | | Cdh17 Sqstm1 Mme Padi3 Mt1a Idi1 Tieg Prss2 Fasn Alpl | |
| | | Atp2a2 Tp53 Cltb Acly Bteb1 Ca3 Gpr48 Gsr Copeb | |
| | | Pkm2 Acsl3 Lrp2 Apobec1 Pklr Limk2 Apex1 Grin2d | |
| | | Adam17 Tf Cdipt Calr Txnrd1 Grin3b Mat1a Rgn Kng1 | |
| | | Alb Znf354a Anpep Sod1 | |
| GO: 0043176 | amine binding | Otc | 0.27 |
| GO: 0046906 | tetrapyrrole binding | Cyba | 0.27 |
| GO: 0048037 | cofactor binding | Por Fasn Bach | 0.82 |
| GO: 0050824 | water binding | Alb | 0.27 |
| Cellular component: | | | |
| GO: 0000267 | cell fraction | Sqle Cttn Apob Slco2a1 Hmgcr Cspg5 Cyp4f2 Srd5a1 Srd5a2 Slc6a9 | 14.61 |
| | | G6pc Ptpn1 Slc1a1 Cdh17 Cyp1a1 Kcna5 Fmo1 Slc17a1 Slc20a2 | |
| | | Scarb2 Atp2a2 Cyp7a1 Scnn1b Ugt1a6 Grp Abcb1 Cyp7b1 Fga | |
| | | Lcn2 Asns Cyb5 Ssr3 Pscd2 Selp Dusp6 Cyp17a1 Cyp2e1 Kcnh2 | |
| | | Smpd3 Slc4a2 Hmgcs1 Pik3c2g Igfals Cxcl10 Hmox1 | |
| GO: 0005578 | extracellular matrix | Timp1 Reil Col1a1 Col3a1 Dcn | 1.62 |
| GO: 0005615 | extracellular space | Ccl2 Igfbp1 Il1a Reil PVR Lbp Afm Fga Rbp4 Cxcl1 Igfals Alb | 3.90 |
| GO: 0005622 | intracellular | Gsto1 Hpcl2 Egr1 Agxt Aldh6a1 Cttn Hnrpa1 Ppp5c Nr0b2 Thrsp | 71.75 |
| | | Ptp4a1 Minpp1 Man2b1 Hrmt1l2 Pdhb Por Socs2 Fabp5 Ebp G10 | |
| | | Pik3r1 Supt5h Apob Ca5a Apoe Hnrpab Myc Lamr1 Csrp2 Eno1 | |
| | | Hmgcr Hspca Sfpq Cspg5 Pdk1 Rpl13 Otc Nqo1 Atf3 Ltb4dh | |
| | | Sc4mol Ap1b1 Cyp4f2 App Rpl22 Nap1l1 Rpl28 Fdft1 G6pc | |
| | | Ptpn1 Cbr1 Pcna Il1a Lgals3 Serpinh1 Jun Cyp1a1 Sqstm1 | |
| | | Rpl3 Rpl6 Zfp36 PVR Rpl13a Tpp2 Mt1a Idi1 Nr1d1 Tieg | |
| | | Myo5b Dkc1 Bad Nfe2l2 Asl Scarb2 Tomm20 Col1a1 Dnch1 | |
| | | Atp2a2 Psma1 Nsfl1c Tp53 Cltb Psma5 Acly Psma7 Bteb1 | |
| | | Ccnd1 Got1 Akr7a3 Tap1 Pax4 Junb Cyp7a1 Atp6v1b2 Eif2b1 | |
| | | Eif2b3 Pitx2 Ca3 Psmb1 Vil2 Psmb3 Psmb4 Psmb5 Abat | |
| | | Zfp36l1 Sfrs5 Cyp7b1 Ap2s1 Gpr48 Ddit3 Aplp2 Psmc2 Phb | |
| | | Psmc3 Gapd Copeb Gsr Eif5 Bdh Eif2s1 Crem Hbb Cat Gtf2f2 | |
| | | Ica1 Smc1l1 Psmd4 Dnajb9 Onecut1 Gusb Ccng1 Cfl1 Lcn2 | |
| | | Srebf1 Bzrp Pkm2 Cyb5 Stat3 Abcd2 Abcd3 Lmna Dncl2a Lrp2 | |
| | | Foxa2 Ptbp1 Tubb5 Ssr3 Baat Ahr Acox2 Sardh Csda Limk2 | |
| | | Apex1 Dusp5 Dusp6 Psen2 Cyba Vhl Nfia Cyp17a1 Nfib | |
| | | Htatip Nr4a2 Gtf2ird1 Cyp2e1 Cdkn1a Gm2a Apaf1 Gadd45a | |
| | | Palm Bcl2l1 Cebpb Ndrg2 Rps2 Hao2 Rps5 Maob Arf4 Bach | |
| | | Calr Col3a1 Txnrd1 Virn Mybbp1a Drpla Prdx6 Mapkapk2 | |
| | | Id1 Id2 Npm1 Id3 Hmgcs1 Irf3 Ncl Gstm1 C1qbp Dia1 Dbp | |
| | | Nfkbia Trib3 Myh3 Pik3c2g H1f0 Rara Pex11a Prss15 Ctsl | |
| | | SMN1 Cited2 Pmpca Ehhadh Pmpcb Oat Hspa8 Znf354a Aldh2 | |
| | | Hmox1 Pck1 Sod1 Avpr1a | |
| GO: 0005964 | phosphorylase kinase complex | Phkg2 | 0.32 |
| GO: 0016020 | membrane | Slc23a1 Pigr Sqle Cttn Erbb3 Slc10a1 Por Ccl2 Ebp Cacna1d Enpp2 | 40.26 |
| | | Pde2a Enpp3 Lamr1 Slco2a1 Hmgcr Cspg5 Npr1 So4mol Itga7 Ap1b1 | |
| | | Cyp4f2 Slc6a11 Gja5 App Srd5a1 Dpp4 Srd5a2 Slc6a9 Fdft1 Pld2 | |
| | | G6pc Slc1a1 Cdh17 Lgals3 Gjb5 Atp1b1 Cyp1a1 Mme Kcna5 Icam1 | |
| | | PVR Fmo1 Slc17a1 Gcgr Bad Slc20a2 Slc12a4 Lbp Alpl Scarb2 | |
| | | Tomm20 Atp2a2 Cltb Tap1 Cyp7a1 Ptpns1 Atp6v1b2 Scnn1b Vil2 | |
| | | Ms4a2 Ugt1a6 Il4r Adora2b Abcb1 Cyp7b1 Ap2s1 Gpr48 Gria1 | |
| | | Trpv1 Aplp2 Phb Trpv5 Tnfrsf1a Tnfrsf1b Abcc6 Abcc9 Slc8a2 | |
| | | Ica1 Cpz Cdc42bpa Fads1 Srebf1 Bzrp Slc3a2 Cyb5 Abcd2 Abcd3 | |
| | | Lmna Lrp2 Ssr3 Pscd2 Selp Gfra3 Lim2 Cd74 Psen2 Cyba | |
| | | Cyp17a1 Grin2d Adam17 Cyp2e1 Palm Ngfr Bcl2l1 Sdc3 Kcnh2 | |
| | | Sdc4 Cdipt Agtr1 Maob Mybbp1a Grin3b Kcnmb1 Slc4a2 Ghr | |
| | | C1qbp Dia1 Pik3c2g Dcc Pex11a Pmpca Anpep Avpr1a | |
| GO: 0043190 | ATP-binding cassette (ABC) transporter complex | Abcd2 | 0.32 |

TABLE 2A
primate toxicology biomarkers
human subset 1 (b)

| | | |
|---|---|---|
| 2 | A2M | alpha-2-macroglobulin |
| 47 | ACLY | ATP citrate lyase |
| 100 | ADA | adenosine deaminase |
| 126 | ADH1C | alcohol dehydrogenase 1C (class I), gamma polypeptide |
| 213 | ALB | albumin |
| 226 | ALDOA | aldolase A, fructose-bisphosphate |
| 706 | BZRP | benzodiazapine receptor (peripheral) |
| 847 | CAT | catalase |
| 1051 | CEBPB | CCAAT/enhancer binding protein (C/EBP), beta |
| 1056 | CEL | carboxyl ester lipase (bile salt-stimulated lipase) |
| 1543 | CYP1A1 | cytochrome P450, family 1, subfamily A, polypeptide 1 |
| 1628 | DBP | D site of albumin promoter (albumin D-box) binding protein |
| 1728 | NQO1 | NAD(P)H dehydrogenase, quinone 1 |
| 1958 | EGR1 | early growth response 1 |
| 2023 | ENO1 | enolase 1, (alpha) |
| 2539 | G6PD | glucose-6-phosphate dehydrogenase |
| 2645 | GCK | glucokinase (hexokinase 4, maturity onset diabetes of the young 2) |
| 2805 | GOT1 | glutamic-oxaloacetic transaminase 1, soluble (aspartate |
| | | aminotransferase 1) |
| 2876 | GPX1 | glutathione peroxidase 1 |
| 2949 | GSTM5 | glutathione S-transferase M5 |
| 2990 | GUSB | glucuronidase, beta |
| 3005 | H1F0 | H1 histone family, member 0 |
| 3043 | HBB | hemoglobin, beta |
| 3162 | HMOX1 | heme oxygenase (decycling) 1 |
| 3312 | HSPA8 | heat shock 70 kDa protein 8 |
| 3479 | IGF1 | insulin-like growth factor 1 (somatomedin C) |
| 3486 | IGFBP3 | insulin-like growth factor binding protein 3 |
| 3552 | IL1A | interleukin 1, alpha |
| 3725 | JUN | v-jun sarcoma virus 17 oncogene homolog (avian) |
| 3726 | JUNB | jun B proto-oncogene |
| 6940 | ZNF354A | zinc finger protein 354A |
| 4023 | LPL | lipoprotein lipase |
| 6578 | SLCO2A1 | solute carrier organic anion transporter family, member 2A1 |
| 4153 | MBL2 | mannose-binding lectin (protein C) 2, soluble (opsonic defect) |
| 4501 | MT1X | metallothionein 1X |
| 4609 | MYC | v-myc myelocytomatosis viral oncogene homolog (avian) |
| 4621 | MYH3 | myosin, heavy polypeptide 3, skeletal muscle, embryonic |
| 4311 | MME | membrane metallo-endopeptidase (neutral endopeptidase, enkepha- |
| | | linase, CALLA, CD10) |
| 4804 | NGFR | nerve growth factor receptor (TNFR superfamily, member 16) |
| 4881 | NPR1 | natriuretic peptide receptor A/guanylate cyclase A (atrionatriuretic |
| | | peptide receptor A) |
| 4953 | ODC1 | ornithine decarboxylase 1 |
| 5054 | SERPINE1 | serpin peptidase inhibitor, clade E (nexin, plasminogen activator |
| | | inhibitor type 1), member 1 |
| 5207 | PFKFB1 | 6-phosphofructo-2-kinase/fructose-2,6-biphosphatase 1 |
| 5313 | PKLR | pyruvate kinase, liver and RBC |
| 5515 | PPP2CA | protein phosphatase 2 (formerly 2A), catalytic subunit, alpha |
| | | isoform |
| 5516 | PPP2CB | protein phosphatase 2 (formerly 2A), catalytic subunit, beta |
| | | isoform |
| 5770 | PTPN1 | protein tyrosine phosphatase, non-receptor type 1 |
| 5914 | RARA | retinoic acid receptor, alpha |
| 5649 | RELN | reelin |
| 6338 | SCNN1B | sodium channel, nonvoltage-gated 1, beta (Liddle syndrome) |
| 6357 | CCL13 | chemokine (C-C motif) ligand 13 |
| 6385 | SDC4 | syndecan 4 (amphiglycan, ryudocan) |
| 6554 | SLC10A1 | solute carrier family 10 (sodium/bile acid cotransporter family), |
| | | member 1 |
| 6522 | SLC4A2 | solute carrier family 4, anion exchanger, member 2 (erythrocyte |
| | | membrane protein band 3-like 1) |
| 6647 | SOD1 | superoxide dismutase 1, soluble (amyotrophic lateral sclerosis 1 |
| | | (adult)) |
| 189 | AGXT | alanine-glyoxylate aminotransferase (oxalosis I; hyperoxaluria I; |
| | | glycolicaciduria; serine-pyruvate aminotransferase) |
| 12 | SERPINA3 | serpin peptidase inhibitor, clade A (alpha-1 antiproteinase, |
| | | antitrypsin), |
| 6890 | TAP1 | member 3 transporter 1, ATP-binding cassette, sub-family B |
| | | (MDR/TAP) |
| 6898 | TAT | tyrosine aminotransferase |
| 7157 | TP53 | tumor protein p53 (Li-Fraumeni syndrome) |
| 1191 | CLU | clusterin (complement lysis inhibitor, SP-40, 40, sulfated |
| | | glycoprotein 2, testosterone-repressed prostate message 2, |
| | | apolipoprotein J) |
| 7276 | TTR | transthyretin (prealbumin, amyloidosis type I) |
| 7428 | VHL | von Hippel-Lindau tumor suppressor |
| 6822 | SULT2A1 | sulfotransferase family, cytosolic, 2A, dehydroepiandrosterone |
| | | (DHEA)-preferring, member 1 |
| 3827 | KNG1 | kininogen 1 |
| 4189 | DNAJB9 | DnaJ (Hsp40) homolog, subfamily B, member 9 |
| 11145 | HRASLS3 | HRAS-like suppressor 3 |
| 6715 | SRD5A1 | steroid-5-alpha-reductase, alpha polypeptide 1 (3-oxo-5 alpha- |
| | | steroid delta 4-dehydrogenase alpha 1) |
| 1491 | CTH | cystathionase (cystathionine gamma-lyase) |
| 3135 | HLA-G | HLA-G histocompatibility antigen, class I, G |
| 1727 | CYB5R3 | cytochrome b5 reductase 3 |
| 10993 | SDS | serine dehydratase |
| 5284 | PIGR | polymeric immunoglobulin receptor |
| 5644 | PRSS1 | protease, serine, 1 (trypsin 1) |
| 3566 | IL4R | interleukin 4 receptor |
| 1571 | CYP2E1 | cytochrome P450, family 2, subfamily E, polypeptide 1 |
| 5338 | PLD2 | phospholipase D2 |
| 3170 | FOXA2 | forkhead box A2 |
| 9104 | RGN | regucalcin (senescence marker protein-30) |
| 552 | AVPR1A | arginine vasopressin receptor 1A |
| 1647 | GADD45A | growth arrest and DNA-damage-inducible, alpha |
| 3290 | HSD11B1 | hydroxysteroid (11-beta) dehydrogenase 1 |
| 6774 | STAT3 | signal transducer and activator of transcription 3 (acute-phase |
| | | response factor) |
| 4645 | MYO5B | myosin VB |
| 4691 | NCL | nucleolin |
| 1586 | CYP17A1 | cytochrome P450, family 17, subfamily A, polypeptide 1 |
| 2974 | GUCY1B2 | guanylate cyclase 1, soluble, beta 2 |
| 4069 | LYZ | lysozyme (renal amyloidosis) |
| 3175 | ONECUT1 | one cut domain, family member 1 |
| 2690 | GHR | growth hormone receptor |
| 1803 | DPP4 | dipeptidylpeptidase 4 (CD26, adenosine deaminase complexing |
| | | protein 2) |
| 2326 | FMO1 | flavin containing monooxygenase 1 |
| 2593 | GAMT | guanidinoacetate N-methyltransferase |
| 3397 | ID1 | inhibitor of DNA binding 1, dominant negative helix-loop-helix |
| | | protein |
| 5825 | ABCD3 | ATP-binding cassette, sub-family D (ALD), member 3 |
| 2180 | ACSL1 | acyl-CoA synthetase long-chain family member 1 |
| 341 | APOC1 | apolipoprotein C-I |
| 632 | BGLAP | bone gamma-carboxyglutamate (gla) protein (osteocalcin) |
| 5245 | PHB | prohibitin |
| 7069 | THRSP | thyroid hormone responsive (SPOT14 homolog, rat) |
| 339 | APOBEC1 | apolipoprotein B mRNA editing enzyme, catalytic polypeptide 1 |
| 467 | ATF3 | activating transcription factor 3 |
| 836 | CASP3 | caspase 3, apoptosis-related cysteine peptidase |
| 900 | CCNG1 | cyclin G1 |
| 1595 | CYP51A1 | cytochrome P450, family 51, subfamily A, polypeptide 1 |
| 1581 | CYP7A1 | cytochrome P450, family 7, subfamily A, polypeptide 1 |
| 2937 | GSS | glutathione synthetase |
| 3383 | ICAM1 | intercellular adhesion molecule 1 (CD54), human rhinovirus |
| | | receptor |
| 3741 | KCNA5 | potassium voltage-gated channel, shaker-related subfamily, |
| | | member 5 |
| 4774 | NFIA | nuclear factor I/A |
| 4792 | NFKBIA | nuclear factor of kappa light polypeptide gene enhancer in B-cells |
| | | inhibitor, alpha |
| 5046 | PCSK6 | proprotein convertase subtilisin/kexin type 6 |
| 140885 | PTPNS1 | protein tyrosine phosphatase, non-receptor type substrate 1 |
| 6193 | RPS5 | ribosomal protein S5 |
| 10060 | ABCC9 | ATP-binding cassette, sub-family C (CFTR/MRP), member 9 |
| 5701 | PSMC2 | proteasome (prosome, macropain) 26S subunit, ATPase, 2 |
| 3399 | ID3 | inhibitor of DNA binding 3, dominant negative helix-loop-helix |
| | | protein |
| 249 | ALPL | alkaline phosphatase, liver/bone/kidney |
| 3398 | ID2 | inhibitor of DNA binding 2, dominant negative helix-loop-helix |
| | | protein |
| 972 | CD74 | CD74 antigen (invariant polypeptide of major histocompatibility |
| | | complex, class II antigen-associated) |
| 5009 | OTC | ornithine carbamoyltransferase |
| 440 | ASNS | asparagine synthetase |
| 7132 | TNFRSF1A | tumor necrosis factor receptor superfamily, member 1A |
| 347265 | KRT8L1 | keratin 8-like 1 |
| 1773 | DNASE1 | deoxyribonuclease I |
| 2538 | G6PC | glucose-6-phosphatase, catalytic (glycogen storage disease type I, |
| | | von Gierke disease) |
| 2280 | FKBP1A | FK506 binding protein 1A, 12 kDa |
| 336 | APOA2 | apolipoprotein A-II |
| 481 | ATP1B1 | ATPase, Na+/K+ transporting, beta 1 polypeptide |
| 3156 | HMGCR | 3-hydroxy-3-methylglutaryl-Coenzyme A reductase |
| 3484 | IGFBP1 | insulin-like growth factor binding protein 1 |
| 196 | AHR | aryl hydrocarbon receptor |
| 1515 | CTSL2 | cathepsin L2 |
| 5950 | RBP4 | retinol binding protein 4, plasma |
| 348 | APOE | apolipoprotein E |
| 5111 | PCNA | proliferating cell nuclear antigen |
| 4129 | MAOB | monoamine oxidase B |
| 11332 | ACOT7 | acyl-CoA thioesterase 7 |
| 22977 | AKR7A3 | aldo-keto reductase family 7, member A3 (aflatoxin aldehyde |
| | | reductase) |
| 4301 | MLLT4 | myeloid/lymphoid or mixed-lineage leukemia (trithorax homolog, |
| | | Drosophila); translocated to, 4 |
| 6132 | RPL8 | ribosomal protein L8 |
| 8618 | CADPS | Ca2+-dependent secretion activator |
| 1634 | DCN | decorin |
| 203068 | TUBB | tubulin, beta |
| 873 | CBR1 | carbonyl reductase 1 |
| 4781 | NFIB | nuclear factor I/B |
| 6713 | SQLE | squalene epoxidase |
| 9391 | WDR39 | WD repeat domain 39 |
| 3921 | RPSA | ribosomal protein SA |
| 1072 | CFL1 | cofilin 1 (non-muscle) |
| 1562 | CYP2C18 | cytochrome P450, family 2, subfamily C, polypeptide 18 |
| 392437 | LOC392437 | similar to hypothetical protein |
| 10124 | ARL4A | ADP-ribosylation factor-like 4A |
| 136 | ADORA2B | adenosine A2b receptor |
| 1466 | CSRP2 | cysteine and glycine-rich protein 2 |
| 677 | ZFP36L1 | zinc finger protein 36, C3H type-like 1 |
| 871 | SERPINH1 | serpin peptidase inhibitor, clade H (heat shock protein 47), |
| | | member 1, (collagen binding protein 1) |
| 1277 | COL1A1 | collagen, type I, alpha 1 |
| 5693 | PSMB5 | proteasome (prosome, macropain) subunit, beta type, 5 |
| 5062 | PAK2 | p21 (CDKN1A)-activated kinase 2 |
| 58 | ACTA1 | actin, alpha 1, skeletal muscle |
| 5447 | POR | P450 (cytochrome) oxidoreductase |
| 7803 | PTP4A1 | protein tyrosine phosphatase type IVA, member 1 |
| 1649 | DDIT3 | DNA-damage-inducible transcript 3 |
| 3929 | LBP | lipopolysaccharide binding protein |
| 1778 | DNCH1 | dynein, cytoplasmic, heavy polypeptide 1 |
| 2065 | ERBB3 | v-erb-b2 erythroblastic leukemia viral oncogene homolog 3 (avian) |
| 5725 | PTBP1 | polypyrimidine tract binding protein 1 |
| 6560 | SLC12A4 | solute carrier family 12 (potassium/chloride transporters), member |
| | | 4 |
| 6575 | SLC20A2 | solute carrier family 20 (phosphate transporter), member 2 |
| 1822 | ATN1 | atrophin 1 |
| 51702 | PADI3 | peptidyl arginine deiminase, type III |
| 3985 | LIMK2 | LIM domain kinase 2 |
| 5631 | PRPS1 | phosphoribosyl pyrophosphate synthetase 1 |
| 1938 | EEF2 | eukaryotic translation elongation factor 2 |
| 388275 | LOC388275 | similar to Heterogeneous nuclear ribonucleoprotein A1 (Helix- |
| | | destabilizing protein) (Single-strand binding protein) (hnRNP core |
| | | protein A1) (HDP-1) (Topoisomerase-inhibitor suppressed) |
| 2222 | FDFT1 | farnesyl-diphosphate farnesyltransferase 1 |
| 2709 | GJB5 | gap junction protein, beta 5 (connexin 31.1) |
| 7832 | BTG2 | BTG family, member 2 |
| 3157 | HMGCS1 | 3-hydroxy-3-methylglutaryl-Coenzyme A synthase 1 (soluble) |
| 162 | AP1B1 | adaptor-related protein complex 1, beta 1 subunit |
| 6430 | SFRS5 | splicing factor, arginine/serine-rich 5 |
| 5682 | PSMA1 | proteasome (prosome, macropain) subunit, alpha type, 1 |
| 5686 | PSMA5 | proteasome (prosome, macropain) subunit, alpha type, 5 |
| 5688 | PSMA7 | proteasome (prosome, macropain) subunit, alpha type, 7 |
| 5691 | PSMB3 | proteasome (prosome, macropain) subunit, beta type, 3 |
| 5702 | PSMC3 | proteasome (prosome, macropain) 26S subunit, ATPase, 3 |
| 9092 | SART1 | squamous cell carcinoma antigen recognised by T cells |
| 708 | C1QBP | complement component 1, q subcomponent binding protein |
| 9562 | MINPP1 | multiple inositol polyphosphate histidine phosphatase, 1 |
| 488 | ATP2A2 | ATPase, Ca++ transporting, cardiac muscle, slow twitch 2 |
| 776 | CACNA1D | calcium channel, voltage-dependent, L type, alpha 1D subunit |
| 570 | BAAT | bile acid Coenzyme A: amino acid N-acyltransferase (glycine |
| | | N-choloyltransferase) |
| 3779 | KCNMB1 | potassium large conductance calcium-activated channel, subfamily |
| | | M, beta member 1 |
| 5534 | PPP3R1 | protein phosphatase 3 (formerly 2B), regulatory subunit B, |
| | | 19 kDa, alpha isoform (calcineurin B, type I) |
| 2702 | GJA5 | gap junction protein, alpha 5, 40 kDa (connexin 40) |
| 6520 | SLC3A2 | solute carrier family 3 (activators of dibasic and neutral amino |
| | | acid transport), member 2 |
| 9963 | SLC23A1 | solute carrier family 23 (nucleobase transporters), member 1 |
| 338 | APOB | apolipoprotein B (including Ag(x) antigen) |
| 351 | APP | amyloid beta (A4) precursor protein (peptidase nexin-II, |
| | | Alzheimer disease) |
| 761 | CA3 | carbonic anhydrase III, muscle specific |
| 763 | CA5A | carbonic anhydrase VA, mitochondrial |
| 4929 | NR4A2 | nuclear receptor subfamily 4, group A, member 2 |
| 1965 | EIF2S1 | eukaryotic translation initiation factor 2, subunit 1 alpha, 35 kDa |
| 7430 | VIL2 | villin 2 (ezrin) |
| 5169 | ENPP3 | ectonucleotide pyrophosphatase/phosphodiesterase 3 |
| 66002 | CYP4F12 | cytochrome P450, family 4, subfamily F, polypeptide 12 |
| 6038 | RNASE4 | ribonuclease, RNase A family, 4 |
| 1983 | EIF5 | eukaryotic translation initiation factor 5 |
| 30819 | KCNIP2 | Kv channel interacting protein 2 |
| 6868 | ADAM 17 | ADAM metallopeptidase domain 17 (tumor necrosis factor, alpha, |
| | | converting enzyme) |
| 7296 | TXNRD1 | thioredoxin reductase 1 |
| 9406 | ZNF265 | zinc finger protein 265 |
| 26227 | PHGDH | phosphoglycerate dehydrogenase |
| 5692 | PSMB4 | proteasome (prosome, macropain) subunit, beta type, 4 |
| 595 | CCND1 | cyclin D1 |
| 8655 | DNCL1 | dynein, cytoplasmic, light polypeptide 1 |
| 1316 | KLF6 | Kruppel-like factor 6 |
| 51582 | AZIN1 | antizyme inhibitor 1 |
| 2870 | GRK6 | G protein-coupled receptor kinase 6 |
| 435 | ASL | argininosuccinate lyase |
| 51602 | NOP5/NOP58 | 58 nucleolar protein NOP5/NOP58 |
| 4000 | LMNA | lamin A/C |
| 3276 | HRMT1L2 | HMT1 hnRNP methyltransferase-like 2 (S. cerevisiae) |
| 2017 | CTTN | cortactin |
| 10514 | MYBBP1A | MYB binding protein (P160) 1a |
| 1183 | CLCN4 | chloride channel 4 |
| 10874 | NMU | neuromedin U |
| 1528 | CYB5 | cytochrome b-5 |
| 5836 | PYGL | phosphorylase, glycogen; liver (Hers disease, glycogen storage |
| | | disease type VI) |
| 64132 | XYLT2 | xylosyltransferase II |
| 3638 | INSIG1 | insulin induced gene 1 |
| 9512 | PMPCB | peptidase (mitochondrial processing) beta |
| 811 | CALR | calreticulin |
| 6175 | RPLP0 | ribosomal protein, large, P0 |
| 6999 | TDO2 | tryptophan 2,3-dioxygenase |
| 27284 | SULT1B1 | sulfotransferase family, cytosolic, 1B, member 1 |
| 334 | APLP2 | amyloid beta (A4) precursor-like protein 2 |
| 4942 | OAT | ornithine aminotransferase (gyrate atrophy) |
| 388 | RHOB | ras homolog gene family, member B |
| 26063 | DECR2 | 2,4-dienoyl CoA reductase 2, peroxisomal |
| 1967 | EIF2B1 | eukaryotic translation initiation factor 2B, subunit 1 alpha, 26 kDa |
| 415116 | PIM3 | pim-3 oncogene |
| 6158 | RPL28 | ribosomal protein L28 |
| 572 | BAD | BCL2-antagonist of cell death |
| 6449 | SGTA | small glutamine-rich tetratricopeptide repeat (TPR)-containing, |
| | | alpha |
| 6716 | SRD5A2 | steroid-5-alpha-reductase, alpha polypeptide 2 (3-oxo-5 alpha- |
| | | steroid delta 4-dehydrogenase alpha 2) |
| 81631 | MAP1LC3B | microtubule-associated protein 1 light chain 3 beta |
| 1175 | AP2S1 | adaptor-related protein complex 2, sigma 1 subunit |
| 343472 | BARHL2 | BarH-like 2 (Drosophila) |
| 1807 | DPYS | dihydropyrimidinase |
| 6202 | RPS8 | ribosomal protein S8 |
| 317 | APAF1 | apoptotic peptidase activating factor |
| 6720 | SREBF1 | sterol regulatory element binding transcription factor 1 |
| 328 | APEX1 | APEX nuclease (multifunctional DNA repair enzyme) 1 |
| 378 | ARF4 | ADP-ribosylation factor 4 |
| 1535 | CYBA | cytochrome b-245, alpha polypeptide |
| 3053 | SERPIND1 | serpin peptidase inhibitor, clade D (heparin cofactor), member 1 |
| 3294 | HSD17B2 | hydroxysteroid (17-beta) dehydrogenase 2 |
| 7538 | ZFP36 | zinc finger protein 36, C3H type, homolog (mouse) |
| 3483 | IGFALS | insulin-like growth factor binding protein, acid labile subunit |
| 3382 | ICA1 | islet cell autoantigen 1, 69 kDa |
| 2920 | CXCL2 | chemokine (C-X-C motif) ligand 2 |
| 635 | BHMT | betaine-homocysteine methyltransferase |
| 18 | ABAT | 4-aminobutyrate aminotransferase |
| 185 | AGTR1 | angiotensin II receptor, type 1 |
| 262 | AMD1 | adenosylmethionine decarboxylase 1 |
| 290 | ANPEP | alanyl (membrane) aminopeptidase (aminopeptidase N, |
| | | aminopeptidase M, microsomal aminopeptidase, CD13, p150) |
| 368 | ABCC6 | ATP-binding cassette, sub-family C (CFTR/MRP), member 6 |
| 471 | ATIC | 5-aminoimidazole-4-carboxamide ribonucleotide formyl- |
| | | transferase/IMP cyclohydrolase |
| 2875 | GPT | glutamic-pyruvate transaminase (alanine aminotransferase) |
| 2963 | GTF2F2 | general transcription factor IIF, polypeptide 2, 30 kDa |
| 4329 | ALDH6A1 | aldehyde dehydrogenase 6 family, member A1 |
| 5138 | PDE2A | phosphodiesterase 2A, cGMP-stimulated |
| 5298 | PIK4CB | phosphatidylinositol 4-kinase, catalytic, beta polypeptide |
| 6137 | RPL13 | ribosomal protein L13 |
| 6143 | RPL19 | ribosomal protein L19 |
| 6146 | RPL22 | ribosomal protein L22 |
| 6747 | SSR3 | signal sequence receptor, gamma (translocon-associated protein |
| | | gamma) |
| 7071 | KLF10 | Kruppel-like factor 10 |
| 7174 | TPP2 | tripeptidyl peptidase II |
| 7431 | VIM | vimentin |
| 60 | ACTB | actin, beta |
| 10399 | GNB2L1 | guanine nucleotide binding protein (G protein), beta polypeptide |
| | | 2-like 1 |
| 3182 | HNRPAB | heterogeneous nuclear ribonucleoprotein A/B |
| 5710 | PSMD4 | proteasome (prosome, macropain) 26S subunit, non-ATPase, 4 |
| 8532 | CPZ | carboxypeptidase Z |
| 4780 | NFE2L2 | nuclear factor (erythroid-derived 2)-like 2 |
| 5078 | PAX4 | paired box gene 4 |
| 3958 | LGALS3 | lectin, galactoside-binding, soluble, 3 (galectin 3) |
| 6799 | SULT1A2 | sulfotransferase family, cytosolic, 1A, phenol-preferring, member 2 |
| 2224 | FDPS | farnesyl diphosphate synthase (farnesyl pyrophosphate synthetase, |
| | | dimethylallyltranstransferase, geranyltranstransferase) |
| 8531 | CSDA | cold shock domain protein A |
| 55968 | NSFL1C | NSFL1 (p97) cofactor (p47) |
| 7442 | TRPV1 | transient receptor potential cation channel, subfamily V, member 1 |
| 5444 | PON1 | paraoxonase 1 |
| 51179 | HAO2 | hydroxyacid oxidase 2 (long chain) |
| 1281 | COL3A1 | collagen, type III, alpha 1 (Ehlers-Danlos syndrome type IV, |
| | | autosomal dominant) |
| 5168 | ENPP2 | ectonucleotide pyrophosphatase/phosphodiesterase 2 (autotaxin) |
| 225 | ABCD2 | ATP-binding cassette, sub-family D (ALD), member 2 |
| 8835 | SOCS2 | suppressor of cytokine signaling 2 |
| 8800 | PEX11A | peroxisomal biogenesis factor 11A |
| 26061 | HPCL2 | 2-hydroxyphytanoyl-CoA lyase |
| 112464 | PRKCDBP | protein kinase C, delta binding protein |
| 3422 | IDI1 | isopentenyl-diphosphate delta isomerase 1 |
| 8896 | G10 | G10 protein |
| 10212 | DDX39 | DEAD (Asp-Glu-Ala-Asp) box polypeptide 39 |
| 9588 | PRDX6 | peroxiredoxin 6 |
| 5689 | PSMB1 | proteasome (prosome, macropain) subunit, beta type, 1 |
| 55512 | SMPD3 | sphingomyelin phosphodiesterase 3, neutral membrane (neutral |
| | | sphingomyelinase II) |
| 8878 | SQSTM1 | sequestosome 1 |
| 26471 | P8 | p8 protein (candidate of metastasis 1) |
| 54578 | UGT1A6 | UDP glucuronosyltransferase 1 family, polypeptide A6 |
| 2181 | ACSL3 | acyl-CoA synthetase long-chain family member 3 |
| 8476 | CDC42BPA | CDC42 binding protein kinase alpha (DMPK-like) |
| 1757 | SARDH | sarcosine dehydrogenase |
| 27297 | RCP9 | calcitonin gene-related peptide-receptor component protein |
| 10370 | CITED2 | Cbp/p300-interacting transactivator, with Glu/Asp-rich carboxy- |
| | | terminal domain, 2 |
| 11140 | CDC37 | CDC37 cell division cycle 37 homolog (S. cerevisiae) |
| 7919 | BAT1 | HLA-B associated transcript 1 |
| 10020 | GNE | glucosamine (UDP-N-acetyl)-2-epimerase/N-acetylmannosamine |
| | | kinase |
| 9446 | GSTO1 | glutathione S-transferase omega 1 |
| 1026 | CDKN1A | cyclin-dependent kinase inhibitor 1A (p21, Cip1) |
| 1843 | DUSP1 | dual specificity phosphatase 1 |
| 3982 | LIM2 | lens intrinsic membrane protein 2, 19 kDa |
| 6536 | SLC6A9 | solute carrier family 6 (neurotransmitter transporter, glycine), |
| | | member 9 |
| 7076 | TIMP1 | TIMP metallopeptidase inhibitor 1 |
| 5163 | PDK1 | pyruvate dehydrogenase kinase, isozyme 1 |
| 3578 | IL9 | interleukin 9 |
| 1212 | CLTB | clathrin, light polypeptide (Lcb) |
| 1978 | EIF4EBP1 | eukaryotic translation initiation factor 4E binding protein 1 |
| 1848 | DUSP6 | dual specificity phosphatase 6 |
| 9672 | SDC3 | syndecan 3 (N-syndecan) |
| 10076 | PTPRU | protein tyrosine phosphatase, receptor type, U |
| 8942 | KYNU | kynureninase (L-kynurenine hydrolase) |
| 2936 | GSR | glutathione reductase |
| 9266 | PSCD2 | pleckstrin homology, Sec7 and coiled-coil domains 2 (cytohesin-2) |
| 5288 | PIK3C2G | phosphoinositide-3-kinase, class 2, gamma polypeptide |
| 3757 | KCNH2 | potassium voltage-gated channel, subfamily H (eag-related), |
| | | member 2 |
| 10961 | ERP29 | endoplasmic reticulum protein 29 |
| 6128 | RPL6 | ribosomal protein L6 |
| 1015 | CDH17 | cadherin 17, LI cadherin (liver-intestine) |
| 26225 | ARL5A | ADP-ribosylation factor-like 5A |
| 622 | BDH1 | 3-hydroxybutyrate dehydrogenase, type 1 |
| 950 | SCARB2 | scavenger receptor class B, member 2 |
| 8431 | NR0B2 | nuclear receptor subfamily 0, group B, member 2 |
| 10682 | EBP | emopamil binding protein (sterol isomerase) |
| 687 | KLF9 | Kruppel-like factor 9 |
| 526 | ATP6V1B2 | ATPase, H+ transporting, lysosomal 56/58 kDa, V1 subunit B, |
| | | isoform 2 |
| 5261 | PHKG2 | phosphorylase kinase, gamma 2 (testis) |
| 6307 | SC4MOL | sterol-C4-methyl oxidase-like |
| 377 | ARF3 | ADP-ribosylation factor 3 |
| 3934 | LCN2 | lipocalin 2 (oncogene 24p3) |
| 5064 | PALM | paralemmin |
| 6579 | SLCO1A2 | solute carrier organic anion transporter family, member 1A2 |
| 116444 | GRIN3B | glutamate receptor, ionotropic, N-methyl-D-aspartate 3B |
| 81285 | OR51E2 | olfactory receptor, family 51, subfamily E, member 2 |
| 23645 | PPP1R15A | protein phosphatase 1, regulatory (inhibitor) subunit 15A |
| 6568 | SLC17A1 | solute carrier family 17 (sodium phosphate), member 1 |
| 1847 | DUSP5 | dual specificity phosphatase 5 |
| 57447 | NDRG2 | NDRG family member 2 |
| 1962 | EHHADH | enoyl-Coenzyme A, hydratase/3-hydroxyacyl Coenzyme A |
| | | dehydrogenase |
| 8891 | EIF2B3 | eukaryotic translation initiation factor 2B, subunit 3 gamma, |
| | | 58 kDa |
| 83449 | PMFBP1 | polyamine modulated factor 1 binding protein 1 |
| 10897 | YIF1A | Yip1 interacting factor homolog A (S. cerevisiae) |
| 55690 | PACS1 | phosphofurin acidic cluster sorting protein 1 |
| 8644 | AKR1C3 | aldo-keto reductase family 1, member C3 (3-alpha hydroxysteroid |
| | | dehydrogenase, type II) |
| 1565 | CYP2D6 | cytochrome P450, family 2, subfamily D, polypeptide 6 |
| 10524 | HTATIP | HIV-1 Tat interacting protein, 60 kDa |
| 22949 | LTB4DH | leukotriene B4 12-hydroxydehydrogenase |
| 10423 | CDIPT | CDP-diacylglycerol--inositol 3-phosphatidyltransferase (phospha- |
| | | tidylinositol synthase) |
| 10965 | ACOT2 | acyl-CoA thioesterase 2 |
| 5504 | PPP1R2 | protein phosphatase 1, regulatory (inhibitor) subunit 2 |
| 3627 | CXCL10 | chemokine (C-X-C motif) ligand 10 |
| 6319 | SCD | stearoyl-CoA desaturase (delta-9-desaturase) |
| 57761 | TRIB3 | tribbles homolog 3 (Drosophila) |
| 9731 | GlyBP | glycine-, glutamate-, thienylcyclohexylpiperidine-binding protein |
| 57546 | PDP2 | pyruvate dehydrogenase phosphatase isoenzyme 2 |
| 6421 | SFPQ | splicing factor proline/glutamine-rich (polypyrimidine tract |
| | | binding protein associated) |
| 8309 | ACOX2 | acyl-Coenzyme A oxidase 2, branched chain |
| 23682 | RAB38 | RAB38, member RAS oncogene family |
| 9572 | NR1D1 | nuclear receptor subfamily 1, group D, member 1 |
| 64816 | CYP3A43 | cytochrome P450, family 3, subfamily A, polypeptide 43 |
| 9804 | TOMM20 | translocase of outer mitochondrial membrane 20 homolog (yeast) |
| 6690 | SPINK1 | serine peptidase inhibitor, Kazal type 1 |
| 173 | AFM | afamin |
| 2760 | GM2A | GM2 ganglioside activator |
| 55829 | SELS | selenoprotein S |
| 7366 | UGT2B15 | UDP glucuronosyltransferase 2 family, polypeptide B15 |
| 55366 | LGR4 | leucine-rich repeat-containing G protein-coupled receptor 4 |
| 55379 | PRO1855 | hypothetical protein PRO1855 |
| 10237 | SLC35B1 | solute carrier family 35, member B1 |
| 5708 | PSMD2 | proteasome (prosome, macropain) 26S subunit, non-ATPase, 2 |
| 5437 | POLR2H | polymerase (RNA) II (DNA directed) polypeptide H |
| 10808 | HSPH1 | heat shock 105 kDa/110 kDa protein 1 |
| 7965 | JTV1 | JTV1 gene |
| 8662 | EIF3S9 | eukaryotic translation initiation factor 3, subunit 9 eta, 116 kDa |
| 51024 | TTC11 | tetratricopeptide repeat domain 11 |
| 83862 | TMPIT | transmembrane protein induced by tumor necrosis factor alpha |
| 28974 | HSPC023 | HSPC023 protein |
| 55827 | IQWD1 | IQ motif and WD repeats 1 |
| 50486 | G0S2 | G0/G1 switch 2 |
| 64005 | MYO1G | myosin IG |
| 344405 | LOC344405 | similar to RIKEN cDNA 2010316F05 |
| 5162 | PDHB | pyruvate dehydrogenase (lipoamide) beta |
| 26354 | GNL3 | guanine nucleotide binding protein-like 3 (nucleolar) |
| 6142 | RPL18A | ribosomal protein L18a |
| 23403 | FBXO46 | F-box protein 46 |
| 3661 | IRF3 | interferon regulatory factor 3 |
| 8726 | EED | embryonic ectoderm development |
| 6157 | RPL27A | ribosomal protein L27a |
| 3665 | IRF7 | interferon regulatory factor 7 |
| 4728 | NDUFS8 | NADH dehydrogenase (ubiquinone) Fe—S protein 8, 23 kDa |
| | | (NADH-coenzyme Q reductase) |
| 282991 | BLOC1S2 | biogenesis of lysosome-related organelles complex-1, subunit 2 |
| 3133 | HLA-E | major histocompatibility complex, class I, E |
| 8870 | IER3 | immediate early response 3 |
| 3109 | HLA-DMB | major histocompatibility complex, class II, DM beta |
| 7327 | UBE2G2 | ubiquitin-conjugating enzyme E2G 2 (UBC7 homolog, yeast) |
| 56241 | SUSD2 | sushi domain containing 2 |
| 90441 | ZNF622 | zinc finger protein 622 |
| 3875 | KRT18 | keratin 18 |
| 710 | SERPING1 | serpin peptidase inhibitor, clade G (C1 inhibitor), member 1, |
| | | (angioedema, hereditary) |
| 344227 | LOC344227 | similar to POTE2A |
| 10480 | hfl-B5 | dendritic cell protein |
| 85441 | PRIC285 | peroxisomal proliferator-activated receptor A interacting |
| | | complex 285 |
| 23203 | PMPCA | peptidase (mitochondrial processing) alpha |
| 11224 | RPL35 | ribosomal protein L35 |
| 8289 | ARID1A | AT rich interactive domain 1A (SWI- like) |
| 9380 | GRHPR | glyoxylate reductase/hydroxypyruvate reductase |
| 123 | ADFP | adipose differentiation-related protein |
| 51154 | C1orf33 | chromosome 1 open reading frame 33 |
| 866 | SERPINA6 | serpin peptidase inhibitor, clade A (alpha-1 antiproteinase, anti- |
| | | trypsin), member 6 |
| 54539 | NDUFB11 | NADH dehydrogenase (ubiquinone) 1 beta subcomplex, 11, |
| | | 17.3 kDa |
| 3320 | HSPCA | heat shock 90 kDa protein 1, alpha |
| 54529 | NBLA00058 | putative protein product of Nbla00058 |
| 6122 | RPL3 | ribosomal protein L3 |
| 143941 | LOC143941 | similar to CDNA sequence BC021608 |
| 84836 | MGC15429 | hypothetical protein MGC15429 |
| 3326 | HSPCB | heat shock 90 kDa protein 1, beta |
| 5558 | PRIM2A | primase, polypeptide 2A, 58 kDa |
| 53938 | PPIL3 | peptidylprolyl isomerase (cyclophilin)-like 3 |
| 9706 | ULK2 | unc-51-like kinase 2 (C. elegans) |
| 11232 | POLG2 | polymerase (DNA directed), gamma 2, accessory subunit |
| 64978 | MRPL38 | mitochondrial ribosomal protein L38 |
| 84861 | KLHL22 | kelch-like 22 (Drosophila) |
| 1974 | EIF4A2 | eukaryotic translation initiation factor 4A, isoform 2 |
| 10188 | TNK2 | tyrosine kinase, non-receptor, 2 |
| 205717 | KIAA2018 | KIAA2018 |
| 387496 | RASL11A | RAS-like, family 11, member A |
| 222229 | DKFZp434K1815 | hypothetical protein DKFZp434K1815 |
| 84876 | FLJ14466 | hypothetical protein FLJ14466 |
| 113878 | DTX2 | deltex homolog 2 (Drosophila) |
| 90378 | SAMD1 | sterile alpha motif domain containing 1 |
| 51057 | LOC51057 | hypothetical protein LOC51057 |
| 39 | ACAT2 | acetyl-Coenzyme A acetyltransferase 2 (acetoacetyl Coenzyme |
| | | A thiolase) |
| 6829 | SUPT5H | suppressor of Ty 5 homolog (S. cerevisiae) |
| 8924 | HERC2 | hect domain and RLD 2 |
| 10527 | IPO7 | importin 7 |
| 6094 | ROM1 | retinal outer segment membrane protein 1 |
| 3117 | HLA-DQA1 | major histocompatibility complex, class II, DQ alpha 1 |
| 3120 | HLA-DQB2 | major histocompatibility complex, class II, DQ beta 2 |
| 55177 | FAM82C | family with sequence similarity 82, member C |
| 126695 | C1orf172 | chromosome 1 open reading frame 172 |
| 54206 | ERRFI1 | ERBB receptor feedback inhibitor 1 |
| 115708 | C14orf172 | chromosome 14 open reading frame 172 |
| 58525 | WIZ | widely-interspaced zinc finger motifs |
| 23325 | KIAA1033 | KIAA1033 |
| 4193 | MDM2 | Mdm2, transformed 3T3 cell double minute 2, p53 binding protein |
| | | (mouse) |
| 90580 | LOC90580 | hypothetical protein BC011833 |
| 80381 | CD276 | CD276 antigen |
| 4794 | NFKBIE | nuclear factor of kappa light polypeptide gene enhancer in B-cells |
| | | inhibitor, epsilon |
| 9188 | DDX21 | DEAD (Asp-Glu-Ala-Asp) box polypeptide 21 |
| 23521 | RPL13A | ribosomal protein L13a |
| 388205 | LOC388205 | similar to RIKEN cDNA 1520401A03 gene |
| 26001 | RNF167 | ring finger protein 167 |
| 5591 | PRKDC | protein kinase, DNA-activated, catalytic polypeptide |
| 5201 | PFDN1 | prefoldin 1 |
| 3337 | DNAJB1 | DnaJ (Hsp40) homolog, subfamily B, member 1 |
| 1555 | CYP2B6 | cytochrome P450, family 2, subfamily B, polypeptide 6 |
| 3418 | IDH2 | isocitrate dehydrogenase 2 (NADP+), mitochondrial |
| 90865 | C9orf26 | chromosome 9 open reading frame 26 (NF-HEV) |
| 5954 | RCN1 | reticulocalbin 1, EF-hand calcium binding domain |
| 51126 | NAT5 | N-acetyltransferase 5 (ARD1 homolog, S. cerevisiae) |
| 90187 | EMILIN3 | elastin microfibril interfacer 3 |
| 5105 | PCK1 | phosphoenolpyruvate carboxykinase 1 (soluble) |
| 4661 | MYT1 | myelin transcription factor 1 |
| 149466 | MGC52423 | hypothetical protein MGC52423 |
| 8664 | EIF3S7 | eukaryotic translation initiation factor 3, subunit 7 zeta, 66/67 kDa |
| 85459 | KIAA1731 | KIAA1731 |
| 148022 | TICAM1 | toll-like receptor adaptor molecule 1 |
| 90809 | TMEM55B | transmembrane protein 55B |
| 5719 | PSMD13 | proteasome (prosome, macropain) 26S subunit, non-ATPase, 13 |
| 10913 | EDAR | ectodysplasin A receptor |
| 196410 | MGC17301 | hypothetical protein MGC17301 |
| 2940 | GSTA3 | glutathione S-transferase A3 |
| 276 | AMY1A | amylase, alpha 1A; salivary |
| 387907 | LOC387907 | similar to bA271B5.1 (similar to ribosomal protein S7) |
| 151963 | LOC151963 | similar to BcDNA:GH11415 gene product |
| 2212 | FCGR2A | Fc fragment of IgG, low affinity Ha, receptor (CD32) |
| 57612 | KIAA1466 | KIAA1466 gene |
| 8848 | TSC22D1 | TSC22 domain family, member 1 |
| 90592 | ZNF700 | zinc finger protein 700 |
| 119504 | C10orf104 | chromosome 10 open reading frame 104 |
| 4717 | NDUFC1 | NADH dehydrogenase (ubiquinone) 1, subcomplex unknown, |
| | | 1, 6 kDa |
| 54566 | EPB41L4B | erythrocyte membrane protein band 4.1 like 4B |
| 6128 | RPL6 | ribosomal protein L6 |
| 54962 | FLJ20516 | timeless-interacting protein |
| 10075 | HUWE1 | HECT, UBA and WWE domain containing 1 |
| 26231 | LRRC29 | leucine rich repeat containing 29 |
| 83636 | C19orf12 | chromosome 19 open reading frame 12 |

human subset 2 (b)

70; 134; 135; 136; 164; 259; 341; 347; 348; 351; 354; 375; 382; 398; 405; 406; 409; 529; 567; 596; 597; 598; 599; 624; 633; 642; 672; 715; 716; 720;
801; 811; 837; 841; 842; 857; 865; 904; 912; 928; 949; 985; 1019; 1020; 1021; 1025; 1026; 1027; 1029; 1032; 1045; 1052; 1054; 1072; 1147; 1176;
1213; 1215; 1270; 1278; 1282; 1284; 1285; 1287; 1288; 1294; 1316; 1385; 1386; 1387; 1409; 1457; 1460; 1499; 1504; 1506; 1508; 1511; 1536; 1543;
1586; 1600; 1630; 1634; 1639; 1647; 1649; 1655; 1676; 1727; 1756; 1773; 1812; 1822; 1956; 1958; 1975; 1981; 1990; 1991; 2002; 2006; 2010; 2033;
2035; 2064; 2071; 2099; 2147; 2153; 2157; 2161; 2192; 2200; 2217; 2237; 2241; 2244; 2245; 2252; 2260; 2263; 2288; 2308; 2309; 2314; 2316; 2335;
2353; 2475; 2537; 2547; 2549; 2560; 2580; 2597; 2638; 2646; 2677; 2820; 2822; 2869; 2885; 2886; 2887; 2908; 2922; 2923; 2934; 2963; 3020; 3028;
3032; 3050; 3055; 3059; 3064; 3065; 3073; 3084; 3091; 3092; 3105; 3107; 3108; 3109; 3122; 3131; 3148; 3159; 3163; 3175; 3190; 3191; 3192; 3202;
3240; 3297; 3303; 3309; 3312; 3320; 3329; 3336; 3337; 3339; 3416; 3429; 3454; 3480; 3483; 3484; 3485; 3486; 3487; 3488; 3489; 3490; 3547; 3551;
3553; 3556; 3559; 3561; 3572; 3577; 3579; 3581; 3586; 3596; 3597; 3609; 3635; 3638; 3661; 3663; 3665; 3667; 3673; 3674; 3675; 3678; 3683; 3684;
3689; 3708; 3716; 3717; 3718; 3725; 3726; 3727; 3732; 3737; 3805; 3817; 3818; 3821; 3822; 3824; 3827; 3831; 3838; 3848; 3925; 3931; 3949; 3952;
3953; 3958; 3959; 3982; 3984; 3990; 4001; 4035; 4036; 4052; 4087; 4088; 4089; 4094; 4099; 4133; 4137; 4147; 4153; 4170; 4193; 4194; 4215; 4216;
4261; 4282; 4284; 4286; 4287; 4303; 4306; 4313; 4318; 4323; 4435; 4478; 4485; 4488; 4547; 4582; 4595; 4602; 4605; 4609; 4615; 4616; 4617; 4632;
4638; 4654; 4664; 4665; 4666; 4670; 4687; 4688; 4691; 4692; 4719; 4738; 4739; 4756; 4773; 4779; 4780; 4784; 4790; 4791; 4792; 4793; 4794; 4801;
4802; 4803; 4811; 4830; 4842; 4846; 4856; 4869; 4892; 4909; 4921; 4999; 5004; 5015; 5027; 5034; 5036; 5047; 5058; 5079; 5111; 5147; 5155; 5164;
5170; 5201; 5208; 5216; 5217; 5241; 5291; 5292; 5295; 5300; 5307; 5310; 5318; 5320; 5327; 5328; 5329; 5337; 5338; 5340; 5348; 5350; 5360; 5366;
5371; 5411; 5422; 5423; 5424; 5425; 5426; 5429; 5430; 5434; 5440; 5441; 5444; 5447; 5449; 5465; 5468; 5469; 5479; 5481; 5499; 5518; 5519; 5520;
5524; 5530; 5534; 5536; 5549; 5551; 5552; 5553; 5566; 5578; 5580; 5581; 5587; 5589; 5590; 5591; 5592; 5594; 5595; 5598; 5599; 5600; 5601; 5602;
5604; 5610; 5611; 5621; 5635; 5645; 5646; 5648; 5663; 5664; 5683; 5684; 5700; 5704; 5705; 5708; 5711; 5716; 5747; 5753; 5757; 5764; 5777; 5781;
5783; 5788; 5818; 5819; 5824; 5825; 5829; 5865; 5868; 5879; 5880; 5883; 5886; 5887; 5891; 5894; 5906; 5909; 5911; 5925; 5927; 5933; 5934; 5948;
5950; 5957; 5966; 5970; 5971; 5981; 5982; 5983; 5984; 5985; 5999; 6010; 6014; 6016; 6117; 6118; 6125; 6135; 6142; 6159; 6189; 6195; 6198; 6237;
6256; 6257; 6261; 6263; 6271; 6272; 6275; 6281; 6282; 6284; 6300; 6319; 6339; 6340; 6383; 6386; 6401; 6403; 6418; 6421; 6426; 6434; 6441; 6449;
6464; 6497; 6525; 6595; 6597; 6598; 6599; 6602; 6620; 6622; 6624; 6654; 6667; 6678; 6688; 6689; 6709; 6714; 6720; 6721; 6722; 6731; 6732; 6738;
6748; 6772; 6774; 6775; 6776; 6777; 6778; 6801; 6804; 6829; 6850; 6872; 6874; 6876; 6877; 6878; 6880; 6885; 6890; 6891; 6892; 6908; 6915; 6925;
6927; 6929; 6938; 6942; 6949; 7015; 7018; 7021; 7025; 7040; 7041; 7042; 7045; 7046; 7048; 7050; 7052; 7056; 7057; 7067; 7074; 7078; 7080; 7082;
7083; 7088; 7094; 7112; 7114; 7124; 7132; 7133; 7137; 7150; 7153; 7157; 7158; 7159; 7161; 7167; 7169; 7170; 7184; 7185; 7186; 7187; 7189; 7203;
7222; 7251; 7253; 7263; 7265; 7276; 7277; 7278; 7280; 7295; 7297; 7319; 7329; 7337; 7341; 7343; 7373; 7374; 7399; 7414; 7415; 7421; 7428; 7430;
7436; 7448; 7450; 7456; 7486; 7490; 7514; 7515; 7518; 7520; 7528; 7529; 7531; 7532; 7533; 7534; 7704; 7707; 7709; 7804; 7818; 7832; 7846; 7850;
7874; 7915; 7916; 7975; 8021; 8029; 8038; 8050; 8061; 8125; 8140; 8178; 8187; 8202; 8204; 8239; 8290; 8295; 8301; 8317; 8370; 8394; 8396; 8431;
8439; 8452; 8464; 8467; 8470; 8505; 8517; 8522; 8550; 8553; 8554; 8555; 8556; 8567; 8570; 8573; 8575; 8600; 8615; 8626; 8648; 8650; 8651; 8655;
8660; 8666; 8669; 8682; 8717; 8728; 8737; 8766; 8767; 8794; 8795; 8818; 8835; 8837; 8840; 8841; 8850; 8874; 8880; 8883; 8892; 8893; 8894; 8900;
8904; 8945; 8976; 8999; 9013; 9014; 9015; 9020; 9021; 9026; 9031; 9043; 9049; 9056; 9063; 9093; 9111; 9112; 9156; 9181; 9188; 9191; 9219; 9221;
9223; 9229; 9230; 9232; 9242; 9256; 9261; 9314; 9325; 9337; 9351; 9368; 9369; 9379; 9401; 9412; 9423; 9439; 9440; 9444; 9451; 9463; 9468; 9479;
9493; 9510; 9530; 9531; 9532; 9540; 9542; 9546; 9564; 9584; 9586; 9601; 9611; 9612; 9618; 9641; 9656; 9672; 9690; 9697; 9701; 9727; 9748; 9804;
9816; 9817; 9863; 9868; 9908; 9924; 9970; 9973; 9978; 9988; 9992; 10001; 10002; 10006; 10010; 10015; 10017; 10018; 10036; 10049; 10053; 10055;
10062; 10075; 10095; 10096; 10116; 10134; 10153; 10155; 10189; 10210; 10212; 10236; 10243; 10250; 10270; 10273; 10288; 10307; 10309; 10376;
10383; 10399; 10401; 10418; 10419; 10423; 10438; 10454; 10456; 10458; 10474; 10477; 10487; 10492; 10498; 10499; 10513; 10514; 10519; 10521;
10524; 10529; 10538; 10575; 10576; 10580; 10598; 10627; 10656; 10657; 10659; 10694; 10714; 10728; 10747; 10808; 10846; 10848; 10856; 10857;
10859; 10891; 10912; 10915; 10928; 10930; 10952; 10953; 10963; 10971; 10985; 10987; 11005; 11016; 11033; 11035; 11036; 11040; 11059; 11060;
11140; 11142; 11146; 11186; 11187; 11236; 11243; 11244; 11277; 11338; 22800; 22808; 22821; 22841; 22853; 22861; 22870; 22883; 22918; 22927;
22937; 22941; 22948; 23013; 23022; 23032; 23041; 23049; 23054; 23059; 23062; 23075; 23077; 23085; 23113; 23118; 23139; 23203; 23214; 23243;
23268; 23291; 23327; 23345; 23373; 23385; 23413; 23428; 23468; 23512; 23523; 23541; 23582; 23621; 23640; 23645; 23647; 23649; 23650; 23657;
23741; 23762; 23764; 23779; 25792; 25898; 25920; 25932; 25942; 25945; 26034; 26052; 26057; 26073; 26088; 26092; 26258; 26287; 26292; 26354;
26502; 26578; 26986; 27000; 27035; 27040; 27085; 27151; 27236; 27252; 27257; 27301; 27335; 27336; 27343; 27429; 27434; 28964; 28996; 29110;
29124; 29883; 29893; 29927; 29967; 29974; 29978; 29982; 30008; 30011; 30818; 30836; 50484; 50626; 50628; 50650; 50805; 50807; 50813; 50848;
51022; 51065; 51074; 51079; 51086; 51102; 51147; 51177; 51191; 51225; 51246; 51386; 51475; 51535; 51621; 51667; 51684; 51701; 51726; 51729;
51741; 51752; 51754; 51762; 51807; 53353; 53358; 53405; 53822; 54205; 54434; 54464; 54700; 54822; 54861; 54882; 54921; 54938; 54971; 55075;
55140; 55176; 55210; 55215; 55250; 55257; 55291; 55294; 55333; 55379; 55388; 55620; 55629; 55690; 55742; 55860; 56142; 56144; 56288; 56301;
56647; 56654; 56896; 56915; 56923; 56993; 57085; 57099; 57111; 57147; 57325; 57448; 57498; 57619; 57670; 57761; 59286; 59341; 60676; 63897;
63922; 63971; 64112; 64215; 64219; 64324; 64326; 64344; 64386; 64689; 64754; 64780; 65078; 79075; 79139; 79444; 79850; 79902; 79960; 80124;
80854; 81532; 81607; 81628; 81928; 83714; 83737; 83874; 83941; 84172; 84289; 84324; 84570; 84617; 84650; 84687; 84817; 84864; 84875; 84894;
84920; 84941; 84951; 84962; 85406; 85415; 85461; 85480; 90480; 90627; 91768; 92609; 92906; 94241; 96764; 117178; 117581; 118424; 118460;
121504; 121512; 121536; 122953; 125168; 125950; 128178; 128240; 130872; 131034; 136227; 137735; 140767; 140890; 143425; 144245; 145258;
149233; 150684; 151531; 152503; 164656; 165904; 168544; 196403; 204851; 221184; 221692; 253980; 255488; 284217; 339403; 347733; 353376;
387082; 388552; 389941; 404734; 407975; 407977

human subset 3 (b)

2; 10; 12; 13; 19; 25; 26; 27; 30; 33; 38; 41; 43; 51; 52; 58; 59; 60; 69; 70; 71; 73; 75; 81; 82; 86; 87; 88; 100; 102; 118; 123; 124;
125; 126; 131; 133; 134; 135; 136; 137; 140; 142; 148; 154; 155; 156; 157; 160; 161; 162; 163; 164; 166; 174; 176; 177; 182; 183; 185;
186; 196; 197; 207; 208; 210; 211; 213; 215; 217; 225; 226; 228; 229; 230; 238; 239; 240; 242; 246; 248; 249; 250; 251; 259; 263; 267;
273; 274; 277; 278; 279; 280; 281; 286; 290; 291; 302; 308; 309; 310; 317; 319; 320; 321; 322; 323; 324; 325; 328; 329; 330; 331; 332;
333; 334; 335; 336; 337; 338; 339; 341; 342; 344; 345; 347; 348; 351; 354; 355; 356; 358; 360; 367; 373; 374; 375; 376; 377; 378; 379;
380; 381; 382; 383; 384; 387; 388; 389; 391; 396; 397; 398; 405; 406; 408; 409; 410; 427; 430; 432; 433; 440; 462; 463; 466; 467; 468;
472; 473; 476; 478; 479; 480; 482; 485; 487; 525; 529; 535; 545; 551; 552; 553; 554; 567; 571; 572; 573; 578; 580; 581; 590; 595; 596;
597; 598; 599; 602; 604; 613; 624; 631; 632; 633; 637; 638; 639; 640; 641; 642; 644; 649; 650; 655; 660; 661; 665; 666; 671; 672; 673;
675; 677; 678; 682; 685; 688; 694; 705; 708; 711; 712; 715; 716; 717; 718; 720; 727; 728; 730; 732; 735; 759; 760; 764; 765; 766; 768;
771; 773; 775; 780; 781; 782; 783; 784; 799; 801; 805; 811; 815; 817; 818; 819; 820; 821; 823; 824; 831; 834; 835; 836; 837; 838; 839;
840; 841; 842; 843; 844; 845; 847; 857; 860; 861; 862; 864; 867; 871; 874; 887; 890; 891; 892; 894; 896; 898; 900; 901; 904; 908; 911;
912; 914; 920; 921; 924; 925; 928; 929; 939; 940; 941; 943; 948; 949; 958; 959; 960; 961; 962; 965; 966; 967; 968; 969; 972; 973; 978;
983; 984; 985; 990; 991; 995; 997; 998; 999; 1000; 1001; 1002; 1003; 1004; 1016; 1017; 1019; 1020; 1021; 1025; 1026; 1027; 1028; 1029;
1030; 1031; 1032; 1033; 1040; 1045; 1048; 1050; 1051; 1052; 1053; 1054; 1057; 1066; 1071; 1072; 1073; 1075; 1080; 1107; 1109; 1111;
1113; 1129; 1131; 1137; 1147; 1174; 1176; 1178; 1179; 1180; 1181; 1182; 1184; 1185; 1186; 1191; 1192; 1195; 1196; 1207; 1211; 1213;
1215; 1230; 1231; 1232; 1233; 1234; 1241; 1244; 1258; 1263; 1264; 1270; 1272; 1277; 1278; 1280; 1281; 1282; 1287; 1288; 1289; 1290;
1291; 1292; 1294; 1297; 1298; 1299; 1301; 1308; 1312; 1315; 1316; 1356; 1360; 1361; 1380; 1381; 1382; 1385; 1386; 1387; 1394; 1397;
1398; 1401; 1409; 1427; 1432; 1435; 1436; 1437; 1438; 1439; 1440; 1441; 1445; 1452; 1453; 1454; 1457; 1459; 1460; 1462; 1464; 1466;
1467; 1490; 1491; 1495; 1499; 1500; 1501; 1504; 1506; 1508; 1509; 1510; 1511; 1512; 1513; 1514; 1520; 1523; 1528; 1529; 1530; 1531;
1532; 1535; 1536; 1537; 1543; 1544; 1545; 1546; 1548; 1549; 1551; 1554; 1555; 1556; 1557; 1558; 1559; 1562; 1564; 1568; 1571; 1574;
1576; 1577; 1580; 1581; 1583; 1584; 1585; 1586; 1588; 1589; 1593; 1595; 1600; 1601; 1604; 1611; 1612; 1616; 1621; 1630; 1634; 1636;
1639; 1642; 1643; 1645; 1646; 1647; 1648; 1649; 1655; 1660; 1662; 1667; 1673; 1674; 1676; 1718; 1719; 1727; 1728; 1737; 1738; 1739;
1742; 1747; 1748; 1756; 1758; 1759; 1773; 1785; 1786; 1791; 1803; 1812; 1813; 1814; 1820; 1822; 1825; 1831; 1832; 1834; 1839; 1843;
1844; 1846; 1847; 1849; 1853; 1854; 1855; 1869; 1874; 1875; 1877; 1889; 1894; 1896; 1902; 1906; 1910; 1915; 1937; 1938; 1950; 1956;
1958; 1959; 1960; 1961; 1964; 1965; 1967; 1968; 1969; 1973; 1975; 1977; 1978; 1979; 1981; 1984; 1989; 1990; 1991; 1993; 1994; 2000;
2001; 2002; 2005; 2006; 2007; 2010; 2017; 2026; 2030; 2033; 2034; 2035; 2048; 2049; 2052; 2056; 2057; 2058; 2059; 2064; 2065; 2066;
2068; 2071; 2073; 2074; 2078; 2081; 2091; 2099; 2100; 2103; 2113; 2114; 2119; 2120; 2125; 2130; 2145; 2146; 2147; 2149; 2150; 2152;
2153; 2157; 2159; 2160; 2161; 2166; 2169; 2180; 2181; 2182; 2185; 2191; 2192; 2194; 2200; 2208; 2209; 2210; 2211; 2212; 2213; 2214;
2215; 2217; 2224; 2237; 2241; 2242; 2244; 2245; 2247; 2252; 2260; 2261; 2263; 2264; 2266; 2272; 2274; 2280; 2281; 2282; 2283; 2284;
2285; 2286; 2288; 2289; 2296; 2297; 2304; 2305; 2308; 2309; 2312; 2313; 2314; 2315; 2316; 2321; 2322; 2327; 2328; 2329; 2330; 2335;
2353; 2354; 2355; 2357; 2395; 2475; 2492; 2494; 2495; 2512; 2516; 2520; 2521; 2534; 2537; 2539; 2542; 2547; 2549; 2551; 2559; 2560;
2569; 2580; 2596; 2597; 2620; 2623; 2624; 2627; 2629; 2638; 2641; 2643; 2644; 2646; 2648; 2660; 2670; 2671; 2677; 2678; 2686; 2688;
2689; 2690; 2697; 2701; 2707; 2720; 2735; 2760; 2761; 2766; 2768; 2769; 2775; 2778; 2782; 2784; 2801; 2805; 2806; 2810; 2811; 2812;
2814; 2815; 2817; 2820; 2821; 2822; 2833; 2868; 2869; 2871; 2872; 2874; 2875; 2876; 2879; 2885; 2886; 2890; 2902; 2903; 2908; 2919;
2921; 2922; 2923; 2932; 2934; 2936; 2938; 2939; 2941; 2942; 2944; 2946; 2947; 2950; 2952; 2956; 2957; 2958; 2959; 2960; 2961; 2962;
2963; 2965; 2966; 2968; 2969; 2972; 2983; 3001; 3002; 3005; 3010; 3014; 3015; 3020; 3028; 3032; 3039; 3040; 3043; 3045; 3046; 3047;
3048; 3049; 3050; 3055; 3059; 3064; 3065; 3066; 3073; 3074; 3075; 3077; 3078; 3082; 3084; 3087; 3090; 3091; 3092; 3093; 3094; 3099;
3105; 3106; 3107; 3108; 3109; 3111; 3112; 3113; 3115; 3117; 3118; 3119; 3122; 3123; 3124; 3125; 3126; 3127; 3131; 3133; 3134; 3135;
3146; 3148; 3156; 3159; 3162; 3163; 3164; 3169; 3170; 3171; 3172; 3175; 3178; 3181; 3182; 3183; 3184; 3187; 3190; 3191; 3192; 3201;
3202; 3206; 3217; 3219; 3221; 3224; 3240; 3241; 3251; 3265; 3275; 3276; 3280; 3283; 3290; 3291; 3292; 3295; 3297; 3301; 3303; 3304;
3306; 3308; 3309; 3312; 3313; 3315; 3316; 3320; 3322; 3323; 3324; 3325; 3326; 3329; 3336; 3337; 3339; 3364; 3375; 3381; 3383; 3384;
3385; 3397; 3398; 3399; 3416; 3422; 3428; 3429; 3431; 3439; 3454; 3455; 3456; 3458; 3459; 3460; 3476; 3479; 3480; 3481; 3482; 3483;
3484; 3485; 3486; 3487; 3488; 3489; 3490; 3491; 3492; 3516; 3547; 3551; 3552; 3553; 3556; 3557; 3558; 3559; 3560; 3561; 3562; 3565;
3566; 3567; 3569; 3570; 3572; 3575; 3576; 3577; 3579; 3581; 3586; 3587; 3594; 3596; 3597; 3600; 3603; 3606; 3608; 3609; 3611; 3621;
3623; 3627; 3630; 3635; 3636; 3638; 3643; 3646; 3651; 3652; 3654; 3655; 3658; 3659; 3660; 3661; 3662; 3663; 3664; 3665; 3667; 3670;
3672; 3673; 3674; 3675; 3676; 3678; 3682; 3683; 3684; 3685; 3688; 3689; 3690; 3691; 3692; 3693; 3695; 3704; 3708; 3713; 3716; 3717;
3718; 3725; 3726; 3727; 3728; 3732; 3735; 3736; 3737; 3738; 3739; 3742; 3745; 3751; 3752; 3753; 3756; 3764; 3767; 3778; 3782; 3783;
3784; 3791; 3800; 3802; 3803; 3805; 3815; 3816; 3817; 3818; 3821; 3822; 3824; 3827; 3831; 3837; 3838; 3845; 3848; 3852; 3855; 3856;
3859; 3861; 3868; 3875; 3880; 3897; 3908; 3912; 3915; 3921; 3925; 3930; 3931; 3932; 3933; 3934; 3939; 3949; 3952; 3953; 3956; 3957;
3958; 3959; 3960; 3964; 3965; 3972; 3976; 3978; 3981; 3982; 3984; 3985; 3990; 3991; 3998; 4000; 4001; 4002; 4003; 4005; 4018; 4023;
4035; 4036; 4043; 4049; 4050; 4051; 4052; 4055; 4057; 4060; 4067; 4068; 4069; 4070; 4072; 4074; 4082; 4084; 4085; 4086; 4087; 4088;
4089; 4091; 4092; 4094; 4097; 4099; 4100; 4123; 4125; 4128; 4129; 4133; 4134; 4137; 4144; 4149; 4151; 4152; 4153; 4155; 4162; 4168;
4170; 4179; 4192; 4193; 4194; 4205; 4209; 4210; 4214; 4215; 4216; 4217; 4221; 4224; 4233; 4237; 4255; 4256; 4261; 4267; 4270; 4276;
4281; 4282; 4283; 4284; 4286; 4287; 4288; 4292; 4294; 4296; 4297; 4298; 4306; 4311; 4312; 4313; 4314; 4316; 4317; 4318; 4319; 4320;
4322; 4323; 4327; 4330; 4331; 4353; 4360; 4361; 4363; 4435; 4436; 4437; 4478; 4485; 4487; 4488; 4489; 4493; 4494; 4495; 4496; 4499;
4500; 4502; 4507; 4512; 4513; 4521; 4524; 4540; 4547; 4582; 4583; 4586; 4595; 4598; 4601; 4602; 4605; 4609; 4610; 4613; 4615; 4616;
4622; 4625; 4626; 4629; 4632; 4633; 4634; 4638; 4640; 4641; 4644; 4653; 4654; 4656; 4664; 4665; 4666; 4670; 4671; 4681; 4683; 4687;
4688; 4689; 4690; 4691; 4692; 4695; 4700; 4701; 4703; 4707; 4709; 4711; 4714; 4719; 4734; 4738; 4739; 4751; 4756; 4760; 4763; 4771;
4773; 4774; 4778; 4779; 4780; 4781; 4782; 4784; 4790; 4791; 4792; 4793; 4794; 4800; 4801; 4802; 4803; 4809; 4811; 4830; 4831; 4835;
4839; 4841; 4842; 4843; 4846; 4851; 4853; 4856; 4860; 4869; 4878; 4879; 4880; 4882; 4883; 4892; 4898; 4899; 4904; 4907; 4909; 4913;
4914; 4915; 4916; 4921; 4923; 4926; 4928; 4940; 4943; 4946; 4947; 4968; 4976; 4982; 4988; 4998; 4999; 5004; 5008; 5009; 5015; 5020;
5021; 5024; 5027; 5029; 5034; 5036; 5045; 5047; 5052; 5054; 5055; 5058; 5062; 5063; 5068; 5071; 5073; 5074; 5076; 5077; 5079; 5092;
5093; 5094; 5104; 5105; 5108; 5110; 5111; 5113; 5122; 5125; 5126; 5136; 5139; 5144; 5154; 5155; 5156; 5159; 5160; 5161; 5162; 5163;
5164; 5165; 5166; 5167; 5170; 5175; 5176; 5178; 5189; 5193; 5201; 5202; 5203; 5204; 5208; 5209; 5216; 5217; 5241; 5243; 5244; 5245;
5246; 5247; 5248; 5249; 5265; 5268; 5271; 5272; 5290; 5291; 5292; 5294; 5295; 5297; 5300; 5307; 5310; 5311; 5315; 5320; 5321; 5325;
5327; 5328; 5329; 5331; 5333; 5335; 5337; 5338; 5339; 5340; 5345; 5347; 5348; 5350; 5355; 5359; 5360; 5366; 5371; 5378; 5395; 5411;
5422; 5423; 5424; 5425; 5426; 5429; 5430; 5434; 5440; 5441; 5443; 5444; 5445; 5446; 5447; 5449; 5451; 5465; 5468; 5469; 5478; 5479;
5481; 5493; 5494; 5499; 5501; 5505; 5511; 5515; 5516; 5517; 5518; 5519; 5520; 5521; 5522; 5523; 5524; 5525; 5530; 5531; 5532; 5534;
5536; 5537; 5540; 5551; 5552; 5553; 5557; 5562; 5563; 5566; 5567; 5569; 5570; 5576; 5578; 5579; 5580; 5581; 5583; 5584; 5585; 5586;
5587; 5588; 5589; 5590; 5591; 5592; 5594; 5595; 5597; 5598; 5599; 5601; 5602; 5604; 5605; 5607; 5609; 5610; 5611; 5612; 5617; 5618;
5621; 5624; 5629; 5630; 5633; 5634; 5635; 5644; 5645; 5646; 5648; 5649; 5651; 5653; 5654; 5657; 5663; 5664; 5681; 5682; 5683; 5684;
5685; 5686; 5687; 5688; 5689; 5690; 5691; 5692; 5693; 5694; 5695; 5696; 5698; 5699; 5700; 5701; 5702; 5703; 5704; 5705; 5706; 5708;
5710; 5711; 5713; 5715; 5716; 5720; 5721; 5724; 5725; 5727; 5728; 5732; 5734; 5741; 5742; 5743; 5745; 5747; 5753; 5756; 5757; 5764;
5770; 5771; 5774; 5777; 5778; 5781; 5783; 5786; 5788; 5791; 5792; 5795; 5797; 5800; 5803; 5804; 5810; 5818; 5819; 5824; 5825; 5826;
5829; 5830; 5858; 5865; 5867; 5868; 5871; 5876; 5879; 5880; 5883; 5884; 5886; 5887; 5888; 5891; 5894; 5896; 5898; 5903; 5906; 5909;
5911; 5914; 5915; 5916; 5921; 5923; 5925; 5928; 5931; 5933; 5934; 5947; 5950; 5957; 5961; 5962; 5966; 5970; 5971; 5972; 5973; 5976;
5978; 5979; 5981; 5982; 5983; 5984; 5985; 5997; 5999; 6010; 6012; 6037; 6041; 6052; 6053; 6054; 6055; 6056; 6059; 6082; 6093; 6095;
6096; 6117; 6118; 6122; 6125; 6129; 6135; 6142; 6154; 6159; 6165; 6176; 6181; 6188; 6189; 6194; 6195; 6196; 6197; 6198; 6199; 6201;
6203; 6204; 6206; 6218; 6233; 6237; 6240; 6241; 6256; 6257; 6259; 6261; 6262; 6263; 6271; 6272; 6273; 6275; 6282; 6284; 6285; 6286;
6288; 6291; 6297; 6300; 6302; 6303; 6311; 6319; 6331; 6337; 6339; 6340; 6346; 6347; 6348; 6351; 6352; 6354; 6355; 6356; 6358; 6361;
6364; 6366; 6367; 6373; 6375; 6376; 6382; 6383; 6386; 6387; 6390; 6399; 6401; 6402; 6403; 6404; 6416; 6418; 6421; 6426; 6427; 6429;
6431; 6432; 6434; 6436; 6441; 6446; 6449; 6462; 6464; 6497; 6508; 6513; 6514; 6518; 6519; 6520; 6521; 6525; 6528; 6531; 6533; 6548;
6550; 6558; 6573; 6574; 6590; 6595; 6597; 6598; 6599; 6602; 6606; 6607; 6612; 6613; 6620; 6622; 6623; 6624; 6626; 6628; 6647; 6648;
6649; 6654; 6655; 6662; 6667; 6670; 6678; 6688; 6689; 6690; 6693; 6696; 6707; 6708; 6709; 6714; 6715; 6716; 6720; 6721; 6722; 6731;
6736; 6738; 6741; 6748; 6750; 6753; 6772; 6773; 6774; 6775; 6776; 6777; 6778; 6780; 6783; 6788; 6789; 6790; 6794; 6801; 6804; 6811;
6812; 6817; 6818; 6820; 6827; 6829; 6833; 6840; 6850; 6853; 6868; 6872; 6874; 6876; 6877; 6878; 6880; 6885; 6890; 6891; 6892; 6908;
6915; 6916; 6921; 6923; 6927; 6928; 6929; 6935; 6942; 6944; 6950; 6955; 6957; 7001; 7008; 7011; 7012; 7015; 7016; 7018; 7019; 7020;
7021; 7022; 7025; 7026; 7027; 7033; 7039; 7040; 7041; 7042; 7043; 7044; 7045; 7046; 7048; 7050; 7052; 7056; 7057; 7064; 7066; 7067;
7068; 7070; 7072; 7073; 7074; 7076; 7077; 7078; 7079; 7080; 7082; 7083; 7088; 7094; 7097; 7098; 7099; 7111; 7112; 7114; 7124; 7128;
7132; 7133; 7135; 7137; 7146; 7148; 7150; 7153; 7155; 7157; 7158; 7159; 7161; 7167; 7168; 7169; 7170; 7173; 7184; 7185; 7186; 7187;
7188; 7189; 7203; 7216; 7222; 7225; 7248; 7249; 7251; 7253; 7263; 7265; 7266; 7274; 7276; 7277; 7278; 7280; 7291; 7292; 7295; 7296;
7297; 7298; 7314; 7316; 7318; 7319; 7321; 7322; 7323; 7324; 7326; 7329; 7332; 7337; 7341; 7343; 7350; 7351; 7352; 7355; 7361; 7362;
7363; 7364; 7365; 7367; 7368; 7373; 7374; 7376; 7378; 7391; 7392; 7399; 7411; 7412; 7414; 7415; 7416; 7421; 7422; 7428; 7430; 7431;
7436; 7439; 7443; 7447; 7448; 7450; 7454; 7456; 7465; 7469; 7471; 7472; 7486; 7490; 7496; 7498; 7507; 7508; 7514; 7515; 7517; 7518;
7520; 7528; 7529; 7530; 7531; 7532; 7533; 7534; 7536; 7538; 7631; 7632; 7633; 7634; 7702; 7704; 7707; 7709; 7764; 7782; 7804; 7812;
7832; 7841; 7846; 7850; 7852; 7874; 7879; 7915; 7916; 7917; 7918; 7919; 7920; 7941; 7975; 7980; 7982; 8013; 8021; 8022; 8029; 8038;
8041; 8048; 8050; 8061; 8065; 8073; 8078; 8081; 8089; 8091; 8125; 8140; 8165; 8178; 8187; 8189; 8202; 8204; 8239; 8260; 8290; 8295;
8301; 8310; 8312; 8315; 8317; 8349; 8379; 8394; 8396; 8410; 8431; 8435; 8439; 8445; 8451; 8452; 8453; 8462; 8464; 8465; 8467; 8468;
8473; 8488; 8489; 8505; 8514; 8517; 8522; 8529; 8536; 8549; 8553; 8554; 8555; 8556; 8557; 8567; 8569; 8570; 8573; 8574; 8575; 8600;
8607; 8611; 8615; 8626; 8637; 8644; 8648; 8650; 8651; 8655; 8659; 8660; 8661; 8666; 8668; 8669; 8674; 8676; 8679; 8682; 8683; 8694;
8717; 8718; 8720; 8728; 8737; 8739; 8740; 8741; 8743; 8751; 8754; 8760; 8764; 8766; 8767; 8772; 8794; 8795; 8797; 8799; 8812; 8815;
8826; 8835; 8837; 8840; 8841; 8850; 8856; 8867; 8870; 8874; 8880; 8883; 8890; 8891; 8892; 8893; 8894; 8900; 8905; 8906; 8907; 8936;
8941; 8943; 8945; 8976; 8999; 9001; 9013; 9014; 9015; 9020; 9021; 9022; 9026; 9031; 9049; 9052; 9055; 9056; 9057; 9077; 9086; 9088;
9093; 9111; 9112; 9134; 9135; 9136; 9140; 9146; 9152; 9156; 9159; 9170; 9173; 9180; 9188; 9191; 9208; 9218; 9219; 9220; 9221; 9224;
9229; 9230; 9232; 9242; 9246; 9252; 9256; 9261; 9265; 9266; 9267; 9294; 9295; 9311; 9314; 9337; 9338; 9343; 9349; 9351; 9355; 9368;
9370; 9378; 9401; 9402; 9414; 9420; 9423; 9429; 9439; 9444; 9445; 9451; 9467; 9468; 9470; 9474; 9476; 9479; 9480; 9493; 9500; 9507;
9510; 9512; 9518; 9530; 9536; 9540; 9542; 9546; 9563; 9564; 9578; 9584; 9586; 9588; 9595; 9601; 9603; 9611; 9612; 9616; 9622; 9641;
9655; 9656; 9659; 9669; 9672; 9690; 9727; 9733; 9746; 9748; 9782; 9804; 9816; 9817; 9833; 9836; 9868; 9908; 9910; 9931; 9962; 9965;
9970; 9971; 9973; 9975; 9978; 9982; 9988; 9990; 9992; 10000; 10002; 10005; 10006; 10017; 10018; 10024; 10036; 10049; 10052; 10053;
10055; 10059; 10062; 10068; 10075; 10095; 10096; 10097; 10110; 10111; 10116; 10123; 10131; 10134; 10138; 10140; 10148; 10153;
10155; 10163; 10168; 10189; 10197; 10203; 10207; 10210; 10212; 10217; 10221; 10226; 10236; 10241; 10242; 10243; 10250; 10252;
10253; 10260; 10267; 10269; 10270; 10273; 10274; 10288; 10294; 10298; 10307; 10309; 10316; 10326; 10335; 10344; 10376; 10382;
10383; 10395; 10397; 10399; 10401; 10413; 10418; 10419; 10420; 10438; 10452; 10456; 10458; 10459; 10474; 10477; 10482; 10487;
10492; 10498; 10499; 10513; 10514; 10519; 10524; 10527; 10528; 10529; 10538; 10544; 10559; 10568; 10575; 10576; 10580; 10597;
10598; 10599; 10611; 10642; 10654; 10656; 10657; 10658; 10659; 10664; 10672; 10694; 10713; 10714; 10723; 10728; 10733; 10746;
10747; 10763; 10766; 10769; 10788; 10805; 10808; 10846; 10847; 10848; 10850; 10855; 10856; 10857; 10858; 10859; 10891; 10892;
10912; 10919; 10928; 10930; 10935; 10941; 10951; 10952; 10953; 10956; 10963; 10970; 10971; 10987; 10988; 11005; 11010; 11016;
11018; 11034; 11035; 11036; 11040; 11060; 11086; 11096; 11127; 11135; 11140; 11142; 11144; 11146; 11156; 11168; 11177; 11186;
11200; 11201; 11221; 11222; 11224; 11238; 11243; 11266; 11275; 11283; 11309; 11315; 11329; 11337; 11338; 22795; 22800; 22803;
22808; 22809; 22821; 22841; 22853; 22861; 22883; 22918; 22926; 22937; 22941; 22943; 22948; 22954; 22976; 22993; 23012; 23020;
23022; 23032; 23049; 23054; 23059; 23062; 23066; 23072; 23077; 23082; 23085; 23113; 23163; 23192; 23193; 23203; 23241; 23283;
23291; 23305; 23327; 23345; 23368; 23373; 23385; 23411; 23413; 23426; 23428; 23433; 23462; 23468; 23480; 23512; 23522; 23523;
23524; 23541; 23582; 23607; 23621; 23627; 23640; 23643; 23645; 23647; 23649; 23650; 23657; 23671; 23678; 23741; 23762; 23764;
23779; 25792; 25803; 25825; 25826; 25897; 25898; 25900; 25920; 25932; 25942; 25945; 25970; 25987; 26003; 26034; 26088; 26092;
26133; 26135; 26191; 26236; 26238; 26258; 26281; 26287; 26292; 26353; 26355; 26502; 26512; 26574; 26578; 26807; 26986; 26999;
27000; 27018; 27035; 27040; 27085; 27097; 27102; 27113; 27128; 27151; 27236; 27238; 27250; 27252; 27301; 27336; 27343; 27429;
27434; 28227; 28231; 28232; 28234; 28951; 28988; 28996; 29072; 29108; 29110; 29124; 29127; 29782; 29843; 29883; 29893; 29904;
29924; 29927; 29940; 29943; 29952; 29966; 29974; 29978; 30000; 30008; 30011; 30818; 30820; 30835; 30836; 30837; 50484; 50486;
50507; 50508; 50617; 50626; 50628; 50631; 50650; 50652; 50805; 50813; 50848; 51022; 51053; 51074; 51079; 51107; 51126; 51129;
51138; 51141; 51143; 51147; 51176; 51177; 51182; 51191; 51199; 51206; 51213; 51225; 51246; 51284; 51287; 51386; 51393; 51429;
51449; 51475; 51512; 51535; 51561; 51588; 51593; 51621; 51667; 51701; 51703; 51726; 51729; 51733; 51738; 51741; 51752; 51754;
51765; 51807; 53353; 53405; 53841; 53905; 53981; 54093; 54106; 54205; 54206; 54211; 54363; 54434; 54476; 54575; 54577; 54583;
54600; 54657; 54658; 54704; 54760; 54766; 54820; 54822; 54861; 54921; 54938; 54971; 55070; 55176; 55269; 55294; 55300; 55333;
55423; 55503; 55509; 55662; 55663; 55664; 55690; 55800; 55851; 55860; 55969; 56301; 56302; 56547; 56616; 56647; 56729; 56848;
56850; 56893; 56899; 56923; 56940; 56993; 57085; 57099; 57110; 57111; 57126; 57147; 57172; 57190; 57325; 57446; 57467; 57468;
57491; 57492; 57498; 57504; 57521; 57546; 57663; 57679; 57718; 57761; 57834; 58155; 58160; 58484; 58492; 59272; 59286; 59341;
59348; 60401; 60495; 60676; 63915; 63922; 63970; 63971; 64084; 64097; 64100; 64109; 64112; 64127; 64131; 64151; 64215; 64219;
64324; 64326; 64344; 64386; 64395; 64421; 64422; 64689; 64708; 64754; 64772; 64780; 64805; 64976; 65009; 65078; 65268; 79009;
79038; 79054; 79075; 79139; 79400; 79424; 79444; 79634; 79658; 79659; 79753; 79796; 79850; 79902; 79960; 80124; 80142; 80155;
80196; 80213; 80263; 80309; 80316; 80331; 80333; 80777; 80781; 80854; 81033; 81502; 81532; 81555; 81572; 81607; 81608; 81628;
81928; 83464; 83528; 83530; 83714; 83737; 83874; 83877; 83931; 83939; 83941; 84099; 84100; 84124; 84168; 84224; 84231; 84260;
84289; 84309; 84324; 84557; 84570; 84643; 84650; 84687; 84698; 84706; 84817; 84823; 84864; 84875; 84894; 84920; 84941; 84951;
84962; 85389; 85390; 85391; 85406; 85415; 85480; 87178; 89845; 90134; 90480; 91419; 91734; 91768; 93664; 94003; 94025; 94241;
96764; 112398; 112399; 113251; 116443; 116519; 116936; 116937; 116938; 117581; 118424; 119391; 121340; 121504; 121512; 121536;
122888; 122953; 122970; 126328; 128178; 128240; 129446; 130872; 132250; 132946; 133150; 135112; 136227; 137735; 138948; 140564;
140735; 140767; 143425; 144245; 145258; 148022; 149233; 149951; 151531; 152503; 153743; 154754; 158172; 158373; 162989; 164656;
165904; 166070; 166994; 168544; 171017; 171024; 171221; 171515; 192146; 199974; 204010; 204851; 221468; 246181; 246182; 253980;
255488; 284217; 284387; 333926; 339403; 344813; 353128; 353269; 353376; 359805; 359806; 386619; 387082; 387521; 389932; 389941;
391538; 406952; 406982; 407975; 407977; 440275; 440738; 441769; 441792; 442219

chimpanzee subset 1 (b)

454672; 461260; 458887; 451116; 469969; 473081; 456184; 468012; 462097; 457913; 454097; 463443; 450978; 470195; 451629; 463396;
470485; 456893; 468739; 464031; 460711; 466126; 460792; 459027; 472467; 469513; 471633; 464100; 469970; 463635; 453984; 469953;
453001; 463890; 453133; 454227; 464075; 456409; 460916; 451279; 456939; 463179; 458883; 457685; 463797; 454005; 450857; 454454;
465598; 452038; 456929; 457698; 453450; 471560; 459689; 468647; 469912; 457037; 461631; 455205; 466721; 462243; 463527; 464191;
450196; 466930; 467426; 454132; 467778; 450337; 458039; 456347; 463632; 456618; 459009; 462191; 463556; 466934; 464613; 453867;
454703; 458013; 457497; 471516; 463395; 450617; 458081; 465588; 469197; 463135; 452120; 471960; 458531; 464995; 464383; 466666;
458071; 471179; 455009; 452089; 452992; 466899; 460965; 463481; 472046; 452017; 451980; 468635; 454181; 464151; 469178; 465800;
452956; 469648; 461892; 458967; 451040; 458383; 454616; 451172; 451338; 455252; 472997; 462258; 457250; 451271; 459058; 464265;
459669; 452980; 450910; 468754; 465205; 453183; 450692; 459018; 467112; 456940; 457168; 457293; 450269; 464324; 471760; 463444;
459883; 457385; 451391; 468580; 455118; 463839; 463631; 455756; 452301; 471216; 451696; 450803; 459054; 465479; 451291; 468658;
456156; 469376; 464330; 470469; 452153; 465200; 464506; 468068; 454269; 463264; 453903; 460762; 467758; 459925; 467506; 451405;
457291; 454316; 454625; 457891; 460799; 472833; 452645; 450133; 462344; 471431; 459769; 472503; 454013; 465289; 469859; 463547;
459815; 466952; 452127; 453641; 460204; 450988; 450263; 463570; 455783; 469589; 473258; 459974; 463445; 463032; 455704; 465094;
450719; 471742; 456520; 459743; 462288; 459641; 456181; 465321; 452266; 461239; 464033; 451869; 464764; 468634; 473385; 466344;
456191; 462487; 460996; 456543; 468022; 466672; 466669; 470228; 451329; 467369; 454028; 460949; 461242; 450676; 469852; 457238;
454147; 451468; 454644; 472456; 471705; 453691; 461289; 451088; 455120; 460887; 471027; 472269; 463482; 457697; 463387; 470377;
470832; 460431; 464992; 456136; 466901; 466438; 450927; 451375; 462547; 458710; 461736; 461302; 458410; 464862; 464727; 456665;
465099; 465012; 456578; 453131; 473588; 470216; 466799; 460411; 462798; 459866; 454507; 454893; 460917; 460954; 471060; 463928;
459270; 453257; 471974; 462595; 467657; 469787; 467566; 455796; 452196; 469058; 467818; 462730; 470439; 450500; 468168; 464165;
462112; 468747; 456052; 453645; 467272; 458256; 456818; 458797; 451482; 465201; 450913; 470472; 462770; 469397; 452476; 450194;
452573; 468729; 452266; 453525; 465650; 454160; 469071

chimpanzee subset 2 (b)

453307; 457638; 458714; 455009; 454228; 460951; 452892; 457279; 451034; 454450; 450170; 458156; 452796; 467547; 473829; 454555;
449497; 451808; 453100; 455756; 451518; 460110; 456457; 463670; 469524; 451775; 452359; 452025; 463891; 463534; 465021; 464163;
468818; 466666; 450673; 471606; 463542; 459988; 465804; 460347; 450269; 467891; 458589; 454179; 463993; 467413; 456888; 455426;
452120; 459332; 456929; 452017; 457928; 458883; 465557; 453867; 471804; 462097; 451925; 460888; 468637; 473591; 465938; 458862;
456689; 454636; 470526; 463079; 451158; 457500; 462304; 458908; 456208; 451242; 461981; 461558; 451748; 454486; 453047; 450178;
458868; 461516; 461352; 461048; 451783; 461270; 459107; 459364; 459670; 462473; 466217; 463410; 471681; 468551; 453387; 457782;
465649; 459080; 467848; 458163; 464259; 452958; 462532; 449592; 462591; 455140; 461606; 462624; 453880; 453450; 464554; 456012;
457867; 463310; 454233; 464733; 451629; 459852; 452179; 468747; 456606; 450608; 453250; 458516; 453676; 463395; 459932; 463396;
454652; 459933; 451929; 465861; 472749; 450200; 460935; 450884; 461866; 470645; 470644; 469657; 455716; 463839; 450927; 454730;
455111; 451958; 458607; 460134; 456911; 455848; 456893; 468739; 466498; 469413; 450205; 469100; 471375; 450156; 460916; 453188;
454841; 456650; 455722; 449638; 456919; 454930; 463466; 467694; 452006; 459713; 459143; 455407; 468537; 469075; 466126; 457274;
457658; 468286; 463119; 453914; 453114; 473659; 461533; 454094; 465711; 460377; 471397; 456539; 463016; 458262; 460269; 467081;
459915; 460640; 451057; 459832; 452434; 455671; 457573; 453273; 459896; 453729; 469972; 455185; 459769; 468742; 461406; 467426;
456014; 462730; 452191; 456799; 455134; 467139; 462261; 466205; 457467; 459868; 465448; 452927; 452318; 467034; 464845; 451439;
458081; 458844; 468402; 462112; 457687; 454456; 451502; 471079; 462656; 461836; 462072; 454340; 465264; 456587; 472748; 450103;
470996; 454454; 458291; 453736; 452875; 465543; 472654; 462720; 456359; 466307; 463547; 463481; 470861; 460178; 454629; 461574;
468983; 466778; 464784; 456150; 469649; 450512; 450503; 451190; 459203; 452834; 455731; 464165; 454037; 450445; 453526; 459155;
458076; 454903; 472555; 465049; 460925; 467503; 457790; 472653; 454777; 460887; 464699; 465803; 464422; 460522; 463759; 467135;
461359; 457607; 457037; 463253; 466893; 473012; 453176; 460182; 469408; 452629; 452715; 467978; 470942; 450617; 455078; 470380;
466665; 456119; 461174; 463467; 452533; 460918; 457470; 454414; 456680; 457025; 464992; 461020; 468955; 464834; 457325; 457320;
457306; 457994; 457324; 450676; 450189; 464298; 464192; 450191; 468417; 466127; 468658; 457351; 470182; 464965; 458702; 451889;
461375; 459171; 451939; 471709; 458229; 462709; 462640; 457597; 465928; 470721; 459834; 454681; 454679; 452005; 470350; 463463;
465701; 451570; 462888; 471978; 468667; 455432; 468650; 453466; 458876; 471696; 471443; 460710; 462758; 461753; 456060; 467962;
457742; 462081; 472992; 460243; 455312; 469898; 453319; 458784; 452867; 453698; 468342; 465080; 452151; 466934; 457968; 472883;
467668; 451799; 465078; 458000; 452187; 464702; 464876; 453178; 450474; 471294; 451065; 458803; 470654; 450779; 455703; 467865;
453269; 459882; 454726; 452217; 457738; 450531; 451858; 464967; 472732; 456094; 471491; 449614; 454409; 458778; 472830; 451557;
460651; 458006; 469648; 451875; 470455; 453907; 449515; 471965; 464807; 450818; 450420; 458308; 463564; 451725; 469380; 457288;
464193; 459983; 461517; 455075; 460136; 467715; 465251; 455640; 461243; 453014; 453714; 469550; 454154; 453523; 464277; 472713;
452127; 459872; 472862; 460219; 452673; 456968; 467995; 453042; 460881; 458188; 467260; 450685; 463688; 454279; 459022; 454750;
472407; 451371; 455403; 453879; 470714; 453217; 457856; 457378; 450500; 457448; 451255; 465215; 462238; 472790; 455154; 464640;
453505; 462213; 454865; 464469; 465356; 455083; 472124; 472181; 459388; 465542; 451148; 453544; 470057; 464116; 462797; 471648;
454246; 463318; 467150; 454534; 471959; 472637; 458987; 453795; 450716; 455707; 470872; 461244; 458014; 451348; 458860; 472434;
470079; 453004; 459685; 467786; 467685; 459487; 455603; 463847; 455710; 456159; 466507; 465650; 463568; 459156; 456353; 455783;
456615; 452976; 456632; 453803; 462116; 462344; 457223; 451037; 452789; 454028; 458845; 457347; 454951; 460154; 459792; 462866;
453647; 450181; 451329; 452070; 453070; 455314; 464413; 456705; 450298; 458498; 451422; 468949; 469007; 461140; 464585; 462168;
458274; 451286; 470302; 466759; 462173; 467007; 463210; 452820; 473600; 464269; 455704; 460392; 464378; 457388; 464368; 451038;
470938; 452684; 472454; 468451; 457923; 458137; 466694; 461721; 456468; 456965; 467937; 467890; 453987; 451020; 452608; 463192;
456568; 464862; 460206; 455443; 463081; 456372; 457435; 452793; 467011; 458709; 458754; 453373; 451573; 456191; 450994; 451318;
451615; 464902; 461248; 473285; 469226; 453759; 460576; 472166; 457514; 461261; 450655; 469288; 460431; 454995; 464532; 465752;
464361; 455828; 457525; 473625; 456009; 465882; 459339; 463923; 454549; 469081; 464331; 466076; 450488; 451335; 465527; 459401;
457034; 472832; 464466; 460466; 461706; 451792; 469555; 453497; 451119; 455877; 456947; 456690; 457268; 455199; 460345; 466683;
451837; 453286; 463884; 460911; 465307; 454258; 471835; 462739; 465522; 460744; 467923; 453429; 460301; 453547; 464081; 450305;
453636; 455378; 458393; 453003; 455120; 466669; 452933; 468817; 450814; 464882; 471713; 470211; 457954; 453297; 457382; 457502;
458114; 459138; 459010; 461235; 463869; 469852; 455689; 452225; 457543; 468427; 471862; 453122; 450344; 471754; 457541; 460278;
458663; 464364; 468131; 454876; 461484; 450944; 457445; 463600; 466918; 458191; 458749; 456899; 459495; 460085; 451971; 455508;
455246; 460541; 472888; 453764; 468760; 467031; 465382; 459678; 455335; 470353; 468014; 465267; 465327; 453048; 455701; 457841;
457399; 459533; 463622; 472838; 466425; 451831; 467546; 459266; 470566; 461537; 460280; 457964; 454111; 454030; 462463; 471605

chimpanzee subset 3 (b)

453133; 460785; 464802; 463895; 457552; 460268; 459914; 451888; 454895; 458990; 466148; 450133; 453307; 454959; 456010; 453472;
465012; 461396; 471257; 451021; 457638; 458714; 455009; 457785; 472723; 471690; 464113; 451363; 458720; 468956; 458967; 454228;
461258; 453629; 462584; 450104; 460762; 460912; 453202; 456035; 464675; 460416; 461260; 452252; 466952; 464623; 459126; 455242;
455103; 470679; 463363; 459590; 457067; 457067; 467758; 453487; 461466; 462198; 452153; 467203; 472947; 453697; 461184; 461999;
457427; 465200; 451508; 451507; 473765; 451696; 451564; 458013; 466792; 459058; 460951; 466149; 469584; 463330; 465045; 465680;
461251; 451869; 464506; 454713; 452892; 462999; 452985; 459054; 457098; 465311; 457279; 451034; 454450; 458946; 464022; 466319;
468172; 463556; 468024; 452429; 468334; 457152; 452489; 468439; 454019; 470401; 454685; 460745; 469863; 452038; 469656; 450170;
458500; 451291; 465041; 462616; 470638; 460830; 458156; 452796; 467547; 473829; 458635; 470232; 462904; 463991; 453650; 454555;
463366; 464042; 458090; 464069; 470705; 449497; 463781; 452526; 452992; 459185; 461250; 452597; 452121; 455252; 451808; 455644;
464703; 468931; 461902; 456887; 464264; 450162; 457915; 473223; 467701; 454624; 451867; 472445; 453100; 459213; 455756; 461445;
470816; 462330; 451317; 457765; 462348; 451519; 463804; 451518; 450745; 460110; 456457; 459873; 451116; 463670; 472017; 451690;
466899; 458533; 466414; 461467; 461835; 462689; 455914; 462243; 461229; 463421; 457419; 469524; 449512; 450124; 451775; 462118;
470617; 470892; 457960; 452359; 473802; 460569; 457437; 457154; 451969; 468440; 465280; 462191; 456066; 450481; 468234; 456819;
456609; 468509; 458380; 461937; 471585; 452025; 463891; 463534; 465021; 461202; 456062; 455931; 469969; 464163; 468818; 467982;
466666; 452853; 451469; 463674; 450274; 463758; 469725; 469999; 450673; 463615; 471606; 472560; 460890; 457955; 453817; 464075;
462564; 465093; 470810; 470781; 450128; 452818; 454126; 455734; 451836; 463542; 459815; 465804; 460347; 462810; 456791; 450717;
458655; 451039; 450269; 452582; 457690; 467729; 467891; 457428; 458589; 460909; 462643; 457127; 462187; 471627; 458975; 454641;
456756; 462182; 454989; 458829; 461766; 453762; 452089; 463003; 456939; 462100; 451203; 454179; 463993; 457677; 467413; 453574;
457278; 457277; 472471; 468580; 468068; 472893; 453745; 456052; 450909; 450626; 450857; 463579; 464191; 464436; 453433; 471972;
463527; 456888; 461905; 471458; 462613; 464827; 455426; 452120; 454782; 459332; 451234; 451165; 456929; 452017; 457568; 470657;
457928; 461773; 458883; 468012; 466780; 460968; 468444; 455109; 465557; 464766; 453867; 455696; 466167; 459689; 471804; 451553;
470885; 455352; 462420; 471248; 471742; 458184; 454158; 454356; 456594; 460853; 464655; 462437; 452613; 472057; 462097; 464048;
452980; 455519; 455218; 451925; 461391; 450886; 460888; 468637; 465023; 455668; 473591; 465938; 451391; 462728; 458862; 459204;
456689; 456613; 457777; 463609; 455728; 473379; 454636; 451980; 459919; 456124; 470526; 452649; 458548; 456031; 463079; 453056;
460906; 473366; 454896; 454697; 451158; 471510; 461800; 457038; 457500; 452679; 462304; 461631; 459974; 464073; 459691; 458908;
454986; 456434; 450194; 457457; 456208; 451242; 461981; 453654; 461558; 461067; 462301; 461560; 470835; 470327; 466649; 451748;
462639; 472990; 452419; 469493; 454486; 464984; 452508; 457505; 469472; 453047; 456127; 459115; 456254; 472945; 470371; 469631;
468262; 450178; 451599; 458868; 461516; 471725; 461352; 472068; 461048; 460599; 451783; 451081; 465619; 460672; 457361; 461270;
459690; 459107; 454677; 454741; 459364; 470168; 458194; 471560; 469273; 460254; 471705; 462453; 463234; 469986; 467989; 468166;
459670; 455941; 462473; 461082; 466217; 455228; 471714; 454396; 471681; 461255; 468551; 453387; 470895; 462769; 462771; 457128;
451717; 459218; 467523; 453483; 460615; 464098; 455639; 451061; 461830; 462549; 463472; 461873; 471879; 466864; 461400; 457782;
465649; 459080; 450978; 466374; 450979; 467848; 458163; 464259; 462950; 461807; 457602; 462489; 463503; 450612; 452958; 461178;
462040; 462532; 450202; 449592; 471979; 471977; 462598; 462599; 471974; 471976; 462591; 462594; 462594; 455140; 462529; 463179;
452518; 461606; 471516; 462624; 470195; 453880; 456143; 458269; 453450; 461216; 462329; 464554; 456012; 457867; 463310; 451951;
454233; 459026; 466302; 458620; 460942; 457698; 468001; 454689; 462019; 450139; 462060; 464733; 451629; 462099; 463488; 451544;
459852; 452179; 468747; 456606; 463402; 471249; 450196; 469912; 459009; 456618; 450608; 450263; 457424; 453250; 470728; 473197;
458516; 458622; 473183; 449517; 470077; 468423; 453676; 466318; 463112; 463395; 459932; 463396; 454652; 459933; 451929; 456988;
461152; 465861; 472749; 470485; 450200; 460935; 459501; 461472; 450884; 470203; 471626; 449565; 454005; 463288; 469506; 461866;
461922; 471205; 470645; 470644; 469657; 451580; 449644; 457328; 455716; 461242; 450954; 451403; 463839; 455649; 464336; 452507;
465935; 459742; 467731; 461629; 462386; 457701; 450927; 454730; 455111; 470596; 451958; 459807; 450396; 458607; 468295; 458200;
451934; 458057; 460134; 456911; 455848; 456893; 468739; 454667; 466498; 454252; 466928; 469413; 469412; 466477; 469965; 465337;
466450; 456098; 450957; 461315; 470557; 450205; 461316; 468971; 469100; 471375; 450156; 460916; 453188; 454841; 473387; 466983;
468241; 454664; 455042; 472122; 456650; 457776; 464914; 464764; 455722; 449638; 456919; 470204; 454930; 456011; 469155; 454519;
468950; 468936; 452653; 463466; 467694; 455448; 457385; 451113; 463116; 464031; 452006; 459713; 461086; 459143; 451778; 460325;
452119; 472771; 459214; 470395; 461661; 450142; 455407; 468537; 467711; 455411; 468359; 469075; 465588; 452972; 455418; 466126;
465888; 457274; 469663; 457658; 467886; 471543; 468286; 463119; 462745; 463671; 465310; 453914; 465979; 471200; 453114; 450866;
471095; 451587; 469111; 461533; 460792; 451510; 454094; 451511; 451509; 458701; 451513; 458730; 452954; 450333; 451488; 465711;
459215; 471494; 460377; 461092; 454105; 471221; 807866; 807864; 463225; 807860; 471397; 456539; 466199; 463016; 458262; 459043;
460269; 468396; 455074; 459915; 460327; 460640; 452002; 457511; 451057; 459832; 452434; 455671; 464278; 457573; 453273; 462119;
458981; 453115; 459871; 471008; 459896; 467689; 453729; 454566; 469972; 455185; 459769; 464995; 455583; 468742; 461406; 467426;
456014; 462730; 452191; 456799; 458869; 455134; 462395; 451784; 473661; 467139; 455026; 462261; 469801; 470677; 465084; 471581;
466205; 456852; 472513; 470311; 462864; 457467; 458392; 451398; 450971; 452267; 460152; 471042; 464351; 472165; 456857; 459868;
465448; 452927; 470020; 452318; 466702; 467034; 453652; 464845; 456537; 472467; 455460; 451439; 460965; 450118; 459349; 452094;
466103; 459300; 467006; 453132; 464020; 463062; 458081; 461740; 464533; 458106; 459797; 465329; 461860; 458844; 461318; 462188;
465525; 471255; 470832; 459743; 468402; 472448; 463002; 454766; 469030; 463532; 468219; 462112; 457446; 458346; 451938; 457687;
450287; 454456; 451502; 455205; 467541; 468566; 455461; 460994; 471079; 462656; 463649; 461836; 462072; 454340; 471394; 456587;
472748; 450103; 460267; 470996; 454454; 453991; 460757; 458291; 453736; 459827; 452875; 465543; 472654; 462720; 456359; 466307;
459074; 463547; 463549; 463548; 463481; 470861; 460178; 454629; 461574; 453893; 452950; 469247; 471633; 464100; 468983; 466778;
462170; 460715; 464784; 454035; 450898; 456150; 466064; 450512; 450503; 451190; 471564; 469367; 472115; 459203; 450143; 456998;
452834; 450288; 455731; 464165; 454037; 450445; 453526; 459155; 462468; 458076; 459593; 457728; 451877; 454903; 463797; 472555;
465049; 460925; 463635; 458478; 450792; 467503; 457790; 451040; 472653; 453569; 452861; 458383; 473258; 469277; 454616; 457293;
454453; 462596; 463632; 451172; 454777; 452909; 460887; 464699; 454240; 465187; 465803; 461963; 469249; 468635; 465254; 466142;
467459; 450195; 466447; 464713; 464422; 451841; 460522; 463759; 469970; 455504; 469639; 467135; 461359; 457607; 450865; 456828;
451181; 465314; 461928; 457037; 453032; 451812; 465371; 469356; 463253; 466893; 461832; 473012; 453339; 453176; 460182; 466489;
459477; 469408; 452629; 451935; 461762; 452715; 456719; 465518; 467978; 450617; 455078; 466776; 470380; 466665; 456119; 469651;
465932; 456373; 471235; 450415; 461174; 463467; 452533; 460918; 469623; 457470; 469607; 461523; 455326; 472956; 454414; 456680;
470216; 457025; 464232; 464992; 455092; 461020; 453545; 450936; 471846; 463127; 450110; 459002; 456282; 469854; 463008; 453592;
459238; 468955; 459024; 464834; 470931; 457325; 457321; 457320; 457994; 457324; 466459; 452024; 465538; 450676; 451776; 450189;
455004; 454579; 468209; 468208; 467985; 459991; 467984; 469572; 454115; 464298; 464192; 456526; 465995; 450191; 452212; 455065;
468417; 453000; 469946; 459167; 466120; 466127; 463011; 468658; 455215; 457351; 459969; 460848; 457916; 459194; 451271; 470182;
468767; 460196; 471620; 459498; 464965; 458702; 451889; 458605; 463937; 461375; 450574; 456047; 470014; 449634; 459171; 452897;
450101; 451939; 459750; 471709; 467939; 461366; 469496; 458229; 462709; 449510; 457597; 459721; 465928; 471035; 458807; 470721;
459834; 454681; 454679; 452005; 458319; 461284; 472829; 458275; 469980; 468641; 470350; 463463; 451268; 464750; 454013; 468422;
451056; 450383; 465606; 459018; 465701; 451570; 462888; 471978; 468667; 463774; 464236; 453843; 468227; 468650; 450387; 458876;
457287; 463368; 468740; 470222; 471443; 460710; 466079; 462758; 470954; 461753; 467769; 470399; 456060; 467962; 457742; 453054;
457780; 462081; 472992; 460243; 455312; 469898; 453319; 451611; 459303; 450776; 454928; 458784; 460176; 452867; 453698; 468342;
465080; 471325; 471374; 464599; 452151; 472136; 466934; 457968; 469636; 472883; 460232; 467668; 451799; 453495; 465078; 458000;
470287; 452187; 464702; 464876; 453178; 457711; 450474; 471294; 451065; 458803; 454674; 472778; 470654; 469586; 450779; 467112;
455703; 468372; 462108; 467865; 452921; 453269; 459882; 454726; 461504; 451416; 461285; 471218; 471219; 461289; 461446; 452217;
472361; 457443; 468835; 457738; 450531; 471632; 451858; 464967; 466630; 453148; 466440; 461071; 451871; 463673; 472732; 470347;
460195; 456094; 453191; 471491; 449614; 454409; 458778; 472830; 466656; 473065; 470886; 451017; 451557; 460651; 453403; 469648;
451875; 470455; 450130; 453907; 470919; 449515; 471965; 462560; 462562; 462563; 462744; 463540; 463672; 464615; 464807; 450818;
466773; 455150; 450420; 458308; 465931; 463564; 451725; 461115; 469380; 469477; 457288; 464193; 452061; 452694; 459983; 466275;
459023; 461517; 472404; 465702; 455075; 455803; 460153; 460136; 467715; 465251; 454633; 469189; 455640; 469196; 461869; 461243;
453014; 456575; 466216; 456714; 453714; 457513; 469550; 452307; 464460; 454154; 469784; 468050; 468687; 455220; 460852; 457354;
464129; 469872; 457221; 453523; 464277; 451389; 472713; 451340; 452127; 459872; 472862; 460219; 462547; 452673; 456968; 467995;
459099; 456543; 453042; 460881; 458188; 467260; 467407; 468761; 462935; 459953; 450685; 463688; 454279; 459022; 454750; 464921;
472407; 451371; 473369; 453656; 456930; 467880; 453879; 470714; 453217; 464292; 455255; 460407; 462011; 454966; 466470; 457856;
451571; 455878; 459462; 450500; 457448; 460050; 451255; 462238; 472790; 466588; 455154; 456181; 454926; 456937; 472627; 464640;
462213; 454619; 454865; 471032; 464469; 464514; 455083; 471251; 472124; 472181; 452714; 459388; 465542; 460015; 462905; 468961;
451148; 468546; 453544; 465641; 457453; 470057; 463631; 468770; 464116; 473071; 471648; 454246; 453180; 463318; 469589; 463298;
467150; 463301; 469101; 459209; 471959; 456642; 459443; 472637; 453795; 467123; 451811; 450716; 462102; 455707; 465095; 470872;
461244; 458014; 452162; 451348; 460230; 458860; 471149; 472434; 470079; 458288; 467685; 459487; 451878; 455603; 463847; 454736;
455710; 456159; 465266; 466507; 465650; 463568; 462054; 460038; 466958; 468406; 455600; 459156; 456353; 456675; 460307; 459810;
455783; 460265; 456615; 455194; 460857; 452976; 456632; 461476; 467708; 460053; 456790; 453803; 460717; 473274; 462116; 458430;
472417; 468685; 464001; 462344; 457223; 451506; 451037; 452789; 469181; 457347; 454951; 457953; 460154; 459792; 451267; 462866;
453647; 451329; 458047; 458198; 471424; 452070; 453070; 465331; 461385; 457350; 464413; 456705; 451173; 450298; 458498; 451422;
461693; 461480; 458865; 461206; 468949; 453154; 469007; 461140; 468549; 464585; 462572; 465865; 468431; 458274; 452022; 451286;
470302; 466759; 452141; 462173; 467075; 467007; 455719; 458108; 452820; 473600; 471354; 450168; 458813; 455704; 458831; 464426;
452855; 460392; 458733; 455429; 460699; 464727; 457388; 458016; 461590; 466711; 465864; 462647; 451038; 470938; 452684; 472454;
468451; 457923; 458137; 457461; 466694; 450465; 461721; 456965; 467937; 467890; 453987; 463367; 452608; 450696; 463192; 454877;
451258; 464862; 462044; 455443; 463081; 453193; 456372; 457435; 466088; 452793; 467011; 450534; 458709; 453842; 458754; 453373;
451573; 469871; 456191; 450994; 451318; 451615; 464217; 464902; 462627; 458562; 464315; 461248; 473285; 469226; 453759; 460576;
472166; 458196; 456928; 457136; 450655; 469288; 452295; 454995; 452729; 454601; 464532; 465780; 465752; 464361; 463255; 455828;
473603; 450735; 457525; 473625; 465882; 459339; 463923; 458390; 453658; 459038; 463925; 460331; 454067; 469081; 451887; 451862;
454350; 456313; 462957; 469177; 473101; 466076; 455748; 451335; 465527; 459401; 471735; 450160; 457034; 472832; 457697; 451709;
472169; 464466; 460466; 461706; 451792; 469555; 462477; 451119; 455877; 457270; 461425; 457268; 450318; 452900; 456429; 473746;
455199; 460345; 451415; 455014; 472172; 470398; 466683; 458984; 451837; 467036; 463860; 453286; 463884; 460911; 465307; 470744;
454258; 471835; 462739; 467613; 458530; 465522; 469787; 460032; 472819; 455552; 451538; 453946; 453429; 460301; 450305; 471160;
453003; 458036; 466183; 466669; 466810; 455962; 452933; 458218; 468817; 450977; 450814; 468943; 457380; 452157; 471713; 462385;
470211; 457954; 453297; 460938; 457382; 457502; 469667; 456649; 458114; 458219; 460295; 458295; 461686; 459010; 459183; 454147;
469031; 470613; 467540; 469852; 459135; 455578; 465511; 455689; 452225; 473648; 457543; 471862; 470944; 453122; 454083; 471153;
450344; 471754; 457541; 470880; 460278; 460010; 468468; 470139; 458663; 450499; 453192; 460037; 464364; 459753; 471318; 456759;
451542; 468131; 454876; 461484; 467145; 453694; 457158; 460119; 473574; 470006; 454202; 469911; 450944; 462162; 457445; 461324;
463600; 467700; 466918; 458191; 458749; 470965; 456899; 460539; 463573; 467870; 452226; 460085; 453885; 451971; 469922; 468409;
465145; 455246; 468657; 460541; 472888; 453764; 468760; 467031; 465382; 459247; 470573; 452031; 455335; 472494; 452850; 452428;
466791; 467004; 465267; 465327; 453048; 453018; 457841; 457399; 459707; 459533; 462982; 463622; 472838; 466425; 451831; 467546;
470566; 461537; 471716; 460280; 451790; 461456; 456556; 457964; 454030; 462463; 457097; 471605; 458330

TABLE 2B
toxicology biomarkers (non-primate)

dog subset 1 (b)
477699; 607852; 477236; 478489; 403550; 479787; 474475; 403474; 491280; 610935; 481528; 479597; 481088; 486834; 442961; 403831; 610251; 476825; 442987;
479406; 610255; 491618; 403782; 609429; 484927; 403626; 485668; 479260; 403800; 403924; 489504; 477120; 491071; 490455; 475666; 403476; 491903; 490425;
403608; 482873; 477263; 480526; 483273; 489972; 445451; 485893; 480372; 475538; 403559; 607355; 480425; 479665; 403869; 442971; 480167; 494000; 478666;
475286; 476045; 403716; 479991; 474479; 486281; 474357; 489957; 415128; 479473; 485742; 480893; 481142; 449023; 490967; 480220; 608445; 477807; 485435;
474442; 487558; 403721; 478767; 403604; 485085; 609779; 479939; 475631; 476437; 403762; 484431; 485169; 486699; 612911; 403567; 479303; 475225; 486962;
442962; 403975; 403995; 479552; 480291; 606906; 403581; 609452; 476366; 486638; 475896; 403547; 403548; 479329; 480878; 475240; 403634; 485387; 403413;
403492; 608169; 478982; 403966; 479182; 480812; 475251; 403708; 477775; 476438; 477166; 403451; 479589; 431695; 475130; 484705; 403904; 474830; 610164;
474709; 608021; 475743; 477029; 609010; 403410; 490748; 485187; 403651; 480246; 612814; 475923; 489816; 481863; 607439; 485869; 480437; 481105; 485097;
479679; 482838; 611608; 478216; 486363; 481011; 477592; 477362; 482488; 488559; 479344; 486344; 476867; 490123; 404305; 480537; 475980; 483721; 489450;
486462; 403878; 484724; 481635; 403983; 612559; 483155; 483777; 478025; 475680; 403407; 487032; 609174; 478754; 480361; 484056; 483393; 474820; 611048;
480442; 403598; 474536; 490213; 607955; 475848; 449028; 609233; 487151; 475058; 479704; 607667; 480124; 476411; 610283; 489442; 491736; 486100; 611347;
476167; 403738; 493990; 475554; 475897; 476694; 475476; 403636; 477858; 403670; 477447; 476378; 483763; 485067; 403715; 479619; 476427; 611092; 475067;
475381; 490004; 481309; 482620; 403475; 482558; 607073; 489664; 489690; 490069; 475242; 479171; 479856; 481960; 403913; 489993; 488513; 609164; 490779;
609574; 483196; 479612; 403682; 479587; 477124; 481992; 477991; 403580; 480818; 607578; 488792; 478813; 482268; 404021; 403892; 480129; 445457; 475234;
475814; 478835; 482026; 477643; 482603; 488736; 477060; 476831; 479743; 476689; 480069; 475040; 489743; 479793; 403629; 478927; 480107; 491277; 479705;
484010; 484955; 474839; 481607; 477813; 474890; 489117; 611737; 482526; 403816; 476828; 489104; 475590; 482594; 487323; 487328; 483907; 475596; 476423;
403761; 477482; 612604; 487050; 488037; 478435; 612125; 611024; 486137; 607275; 475491; 486562; 491320; 485098; 403520; 485092; 485238; 476419; 486884;
609390; 488110; 475379; 479670; 476014; 483716; 497070; 415120; 483728; 474802; 489940; 490770; 478605; 478432; 486839; 485819; 607774; 489771; 475340;
608181; 489851; 608433; 610385; 475175; 479324; 607791; 480777; 476896; 480549; 478654; 478656; 477322; 479752; 479766; 479728; 479721; 610870; 612864;
480085; 476574; 476672; 480221; 612248; 476412; 476779; 479004; 612865; 481708; 607827; 611581; 486402; 479383; 477601; 475966; 475950; 485973; 480677;
480729; 487352; 612041; 474720; 487406; 490838; 480894; 480438; 478838; 475009; 489382; 484744; 474950; 607430; 490910; 483320; 486433; 488118; 488025; 487980; 486032; 479725; 486261;
479723; 474613; 484063; 476462; 479005; 485383; 483786; 474861; 610049; 487497; 612121; 489636; 490871; 476682; 474533; 403693; 611185; 487638; 611638; 476891; 474176; 606872; 476688;
474177; 479043; 485379; 403810; 475952; 477138; 485877; 403560; 485987; 610810; 608665; 611852; 482559; 475934; 481305; 474390; 610194; 474938; 608392; 608201; 476927; 608546; 608342;
607943; 474797; 607505; 612604; 608546; 480931; 489762; 484611
dog subset 2 (b)
478250; 403961; 403960; 403410; 479666; 481685; 476437; 478604; 476438; 403407; 480487; 490679; 611541; 483185; 476860; 607262; 477740; 478284; 403416;
488770; 403618; 403658; 403905; 480635; 403437; 477707; 486714; 480416; 476694; 403724; 488473; 487432; 403980; 479690; 486564; 611695; 486241; 481131;
475537; 609920; 491325; 474890; 403429; 481563; 611216; 486028; 609010; 477796; 607494; 480578; 490628; 483464; 403824; 403496; 403842; 403466; 403840;
403467; 487151; 607922; 478806; 479866; 487786; 477185; 474844; 477032; 479649; 611100; 486077; 608543; 491825; 477807; 610344; 483976; 403904; 483106;
607439; 480472; 487449; 474479; 606758; 403413; 489110; 611608; 404306; 481528; 477600; 403515; 442980; 491860; 442955; 403883; 476105; 403640; 403875;
489090; 474468; 478293; 476414; 476063; 403754; 475472; 491901; 403915; 475582; 415125; 477726; 477295; 481084; 403845; 490792; 478232; 478170; 474485;
483808; 403755; 475172; 475705; 475769; 478755; 478735; 486915; 483312; 491021; 478047; 478279; 474819; 609164; 480110; 480930; 607926; 485828; 479074;
487309; 487633; 482861; 480348; 489814; 474836; 481732; 607827; 481731; 609564; 486068; 442946; 479864; 487558; 480979; 476466; 480100; 482370; 479668;
475124; 403612; 480726; 479406; 480438; 478854; 476688; 612371; 477768; 609830; 442951; 490069; 480812; 488516; 491618; 608158; 610316; 607374; 608559;
481051; 482839; 403974; 488126; 403870; 403851; 403545; 478906; 478905; 611642; 403628; 442990; 403623; 484950; 475554; 476412; 482264; 486148; 489208;
403789; 486493; 489905; 489928; 476548; 442952; 484185; 610004; 609429; 484927; 609409; 404022; 403967; 475624; 611325; 611360; 478666; 480446; 608024;
444857; 478175; 479680; 403616; 450207; 404021; 483345; 611737; 489800; 478320; 474663; 481124; 478781; 475720; 480144; 610902; 476196; 611884; 488504;
480488; 491431; 479260; 403537; 403693; 478939; 480481; 476262; 490008; 607104; 415126; 480229; 491946; 475461; 403733; 403885; 403823; 607904; 491924;
484768; 403663; 487873; 448784; 482515; 483995; 477232; 403924; 477024; 485069; 482582; 478896; 488012; 611940; 488447; 481122; 613013; 476725; 607700;
480036; 608445; 488693; 478880; 480265; 488220; 487635; 485930; 403692; 478813; 484920; 442859; 486858; 480291; 484519; 475450; 475312; 403402; 488958;
480558; 477498; 403784; 475083; 608196; 477823; 611987; 478987; 478868; 490708; 448778; 483369; 474396; 491264; 607885; 612021; 477166; 403781; 442986;
491075; 479875; 606872; 480011; 403621; 477085; 481772; 487235; 484963; 481513; 606755; 486613; 482840; 403426; 476446; 488167; 479473; 403602; 476487;
485903; 611484; 403907; 480869; 494001; 610047; 482686; 486223; 474915; 479483; 611859; 475234; 489816; 403753; 403606; 480533; 478337; 475481; 403609;
484335; 479444; 477374; 491303; 403486; 612559; 612199; 488561; 489030; 609421; 403556; 490904; 494005; 609934; 609091; 484941; 479577; 474176; 477575;
486758; 489532; 477746; 607019; 474652; 478464; 478347; 611397; 476966; 485783; 608323; 475521; 488121; 403408; 490382; 475870; 480338; 478703; 476459;
480478; 478654; 474703; 481014; 475108; 610128; 475509; 486717; 477488; 403662; 490255; 489363; 611060; 478972; 479939; 608161; 404008; 403955; 483696;
484922; 481656; 490863; 484648; 483225; 476968; 476915; 477727; 477221; 478127; 477089; 477775; 489500; 474606; 483729; 484454; 479112; 489798; 477308;
478667; 477499; 488666; 493763; 480126; 491196; 487339; 479949; 475080; 476672; 476601; 403677; 487353; 480580; 476413; 491278; 481735; 480141; 479915;
403787; 475851; 475852; 490457; 607023; 486839; 489598; 489973; 477945; 482977; 403999; 403994; 608403; 475340; 609693; 478663; 489066; 485067; 490439;
489610; 477542; 476335; 476710; 486407; 476640; 486548; 479279; 478478; 489880; 483042; 486507; 612159; 611255; 403484; 485864; 403475; 481228; 612861;
403944; 474887; 478957; 481082; 488449; 490967; 478845; 490968; 490969; 607840; 476462; 484196; 491950; 477280; 479424; 489846; 480815; 474864; 481740;
611193; 485056; 403949; 486303; 485079; 481224; 403592; 403412; 477072; 403463; 488908; 403998; 489932; 488596; 481519; 481628; 477039; 608918; 485867;
474355; 487486; 403601; 487731; 481289; 403940; 403752; 483343; 476314; 474759; 482618; 403922; 403634; 487437; 403566; 477229; 480525; 403869; 478274;
490397; 489620; 477711; 481598; 484695; 404019; 612709; 480673; 490867; 480123; 483844; 485406; 403968; 481275; 489152; 480167; 478918; 610636; 478701;
476714; 492095; 479010; 478874; 480503; 475085; 486315; 488593; 479252; 481590; 486588; 494000; 484056; 476336; 480621; 399544; 488409; 493997; 609253;
474609; 476447; 488923; 478902; 611171; 477237; 480645; 489818; 475864; 489398; 488017; 607083; 489584; 480127; 488559; 608147; 481330; 479854; 488246;
481713; 489897; 608534; 403767; 475942; 483724; 403534; 489667; 484820; 485915; 478385; 479747; 476780; 479955; 491034; 612125; 477892; 477392; 474922;
475451; 477749; 608189; 477480; 484680; 478350; 476294; 479926; 475982; 485022; 480887; 488423; 609418; 478430; 488125; 475684; 480381; 490006; 609233;
485548; 612971; 487532; 610113; 611262; 489156; 488195; 403957; 477936; 482603; 488471; 482048; 478040; 477052; 490201; 610026; 490789; 488103; 477197;
477302; 477213; 486851; 475213; 478428; 609622; 608850; 490926; 442949; 486251; 479719; 480557; 610467; 490602; 480147; 479865; 476146; 480450; 490370;
403498; 476891; 488649; 476057; 609496; 476565; 480194; 611619; 479307; 486992; 491112; 490263; 481657; 478340; 489162; 479884; 483299; 490812; 483760;
482101; 477664; 489497; 474822; 476769; 606754; 475756; 610191; 491780; 483640; 478355; 607954; 486916; 610506; 610825; 487158; 485053; 479648; 477216;
475266; 482715; 479515; 486242; 478950; 474829; 487323; 482822; 481191; 486876; 479207; 476713; 475904; 478541; 478429; 474404; 488653; 442988; 474494;
475217; 403471; 490763; 487629; 607247; 608092; 477015; 612867; 485038; 608937; 611211; 476425; 483625; 480931; 479744; 403702; 478352; 481080; 483035;
403689; 481577; 476689; 478192; 490739; 478185; 474362; 479892; 484309; 606955; 608864; 491231; 480818; 483160; 476864; 480242; 489940; 612549; 481245;
480134; 403473; 476541; 612823; 475317; 474986; 484947; 486989; 480588; 489442; 479044; 474508; 483728; 477974; 611951; 482271; 474445; 480402; 607207;
475106; 487316; 607310; 478399; 476806; 612760; 477322; 484073; 476418; 481029; 479127; 484198; 478052; 490548; 481788; 404018; 477239; 474837; 607060;
477505; 477901; 478055; 607149; 606942; 608486; 442959; 477930; 479672; 484955; 610925; 479951; 607929; 483399; 609422; 612152; 475089; 608504; 476865;
477082; 476998; 477834; 608946; 489449; 484298; 479600; 606923; 484133; 606873; 477351; 479984; 481141; 479832; 477202; 479868; 608555; 485385; 476952;
486753; 481809; 484028; 482513; 480677; 477058; 477794; 479291; 476189; 484037; 488638; 491294; 490608; 477593; 491158; 486403; 477539; 607896; 489390;
476381; 476419; 610132; 476025; 483821; 608367; 477541; 481255; 480709; 612746; 491230; 487367; 478372; 490341; 491162; 481266; 610317; 478288; 486822;
475323; 480819; 484161; 612751; 492016; 607947; 609080; 484845; 475467; 480067; 475587; 491499; 476468; 481062; 608341; 491180; 481145; 482895; 607659;
404006; 481996; 477581; 491909; 479220; 476020; 612797; 609135; 490154; 481988; 609854; 611189; 476579; 482044; 486722; 478974; 478956; 610569; 475943;
476659; 479988; 478159; 477712; 607927; 478474; 491068; 609054; 474513; 611821; 477639; 488698; 475542; 608531; 491160; 478664; 609794; 610120; 488898;
486762; 476136; 481822; 475258; 477526; 477100; 479838; 478300; 477011; 612748; 484518; 479618; 403704; 477370; 478007; 488731; 480158; 483688; 475465;
490757; 487128; 485041; 483716; 476859; 480337; 607224; 607199; 477953; 484599; 477864; 480668; 483004; 611350; 489170; 610117; 608333; 478243; 490418;
480077; 477146; 475718; 475657; 478438; 482778; 485819; 611310; 486308; 480058; 480598; 490084; 610516; 607587; 480949; 489094; 480060; 476429; 490046; 485594; 481958; 609036; 475086;
485969; 480642; 475913; 483830; 486980; 476079; 611494; 609931; 479734; 612380; 486744; 477199; 486350; 479551; 607562; 610442; 480213; 476910; 491076; 478533; 487660; 484852; 491012;
480244; 474392; 403518; 478760; 607671; 476693; 477311; 476461; 475729; 611710; 477885; 479970; 606935; 489713; 486615; 486656; 611936; 607372; 612116; 485661; 608184; 481999; 478734;
487233; 485365; 609732; 403590; 482645; 608593; 485597; 482787; 475803; 478115; 478723; 489942; 488237; 478702; 612307; 606995
dog subset 3 (b)
477699; 480425; 477115; 481651; 491292; 475536; 480052; 477023; 478895; 489421; 477612; 489828; 483322; 475923; 477587; 403580; 478250; 608134; 484526;
478637; 480369; 479191; 477236; 478321; 488796; 474720; 478489; 403817; 403961; 403960; 403410; 403805; 490385; 403866; 403910; 442979; 483701; 486327;
484375; 475994; 486344; 480605; 479666; 485064; 403551; 403828; 403168; 485769; 403783; 403835; 475251; 607538; 490878; 449021; 474808; 476600; 403550;
612520; 610941; 477643; 479787; 474787; 480622; 483021; 479476; 477753; 489486; 489458; 403736; 403548; 481685; 475877; 483870; 607460; 607460; 482842;
403913; 475630; 403435; 476094; 479325; 479246; 482620; 474405; 488700; 476833; 488831; 479139; 482558; 479464; 489433; 442989; 442936; 476478; 479427;
478982; 489392; 475680; 486699; 476437; 442969; 478604; 476438; 403407; 486469; 442968; 403732; 611792; 403588; 487232; 475176; 480487; 486562; 607073;
490950; 475206; 490679; 474823; 480364; 403954; 403670; 483218; 485217; 475924; 486666; 611541; 483185; 476860; 485189; 607262; 474457; 482897; 483666;
489461; 475240; 480066; 489729; 486545; 612911; 474503; 479450; 479596; 403992; 612463; 403820; 488637; 489479; 479797; 474628; 477740; 607705; 477101;
403579; 481142; 488578; 403804; 478284; 487718; 483763; 611781; 481744; 610190; 403523; 488143; 449028; 403416; 488770; 403618; 612349; 488124; 607482;
403658; 485756; 403762; 403905; 486607; 481947; 486080; 480635; 475867; 486127; 477162; 477270; 491750; 609056; 608552; 477226; 403437; 475526; 474180;
490748; 609810; 482682; 612788; 489450; 478194; 477707; 486714; 476728; 442974; 489221; 479555; 479354; 487031; 477928; 403503; 489772; 611933; 478333;
403516; 474832; 610342; 491030; 487113; 486563; 482306; 480416; 474584; 476694; 612941; 610764; 489050; 474684; 442947; 403908; 483745; 480118; 479148;
482737; 403567; 403724; 487899; 486896; 488473; 487432; 488472; 608401; 483134; 403474; 403980; 487746; 487044; 487366; 489369; 485187; 487748; 485333;
483845; 485338; 475007; 611782; 608847; 484610; 479303; 478442; 486564; 488609; 607448; 403931; 612338; 607675; 403157; 611695; 607076; 611674; 403646;
403765; 487438; 475931; 486241; 403469; 403468; 478552; 488642; 475945; 474391; 489476; 477698; 479329; 484483; 487393; 488997; 480527; 482534; 610456;
403934; 442858; 480169; 611010; 489775; 479376; 489256; 481131; 475537; 609920; 491325; 474890; 403429; 481563; 475359; 611216; 609346; 487840; 486028;
490607; 403643; 609010; 490649; 478608; 492302; 609767; 403858; 403827; 485972; 477796; 607494; 490178; 403723; 488106; 477354; 491882; 478229; 479889;
442971; 474847; 490427; 403971; 474765; 480578; 490628; 484791; 484790; 448802; 403541; 484789; 608461; 403632; 611173; 475380; 484937; 483464; 477641;
403651; 403824; 403826; 478835; 403496; 403466; 403840; 480684; 478836; 403668; 403467; 481873; 607622; 403739; 445450; 476866; 487151; 442963; 403512;
608910; 490269; 612093; 607922; 478806; 479866; 610380; 612710; 487786; 607506; 403856; 611795; 489188; 403923; 609266; 481276; 608246; 607605; 487649;
479331; 475928; 474510; 477185; 478108; 474844; 488922; 487658; 479991; 477032; 483489; 488048; 479649; 611100; 486077; 483662; 488577; 608543; 479065;
608843; 403400; 476167; 489664; 491825; 475121; 494010; 483038; 484494; 484494; 479740; 474177; 415122; 415128; 479740; 415127; 608452; 486962; 478365;
482071; 477807; 494003; 415124; 610489; 475225; 610344; 479353; 609307; 607300; 484142; 474873; 448806; 483976; 403904; 610668; 483106; 476067; 483626;
607439; 480472; 480032; 479448; 478924; 487449; 489573; 609048; 474479; 610935; 489406; 403978; 488036; 489462; 491073; 609214; 606758; 491319; 403413;
484949; 476715; 486815; 478767; 489110; 403701; 487984; 611608; 492057; 487849; 607007; 489117; 611653; 482880; 486884; 484742; 609526; 489593; 485839;
611243; 611103; 487388; 488172; 491935; 474800; 403424; 403862; 403506; 611396; 403657; 404306; 481528; 479219; 486121; 483112; 611715; 480361; 477447;
480866; 487421; 479485; 477600; 487870; 475590; 489027; 489468; 403515; 442980; 481565; 480466; 492129; 608908; 491860; 488574; 610283; 477709; 442966;
474578; 442955; 487380; 488108; 480113; 404002; 484943; 478962; 477686; 403883; 481105; 488508; 493995; 476105; 485540; 477747; 610766; 476460; 403640;
403639; 490798; 489287; 487761; 607474; 486685; 483324; 609786; 480516; 475511; 488942; 488940; 490153; 403875; 476993; 489090; 482510; 487924; 475631;
478927; 481018; 486102; 608176; 474468; 483378; 478293; 403462; 478984; 476414; 480129; 476063; 403754; 609483; 475472; 491901; 403915; 475582; 488808;
415125; 489095; 475474; 474544; 608169; 612965; 483764; 477726; 481759; 486743; 477295; 489286; 403495; 481084; 403727; 486025; 480076; 403603; 490345;
475819; 403845; 490792; 484445; 608412; 478232; 481367; 490252; 479746; 609642; 475032; 478170; 481088; 489375; 474485; 483808; 478394; 489145; 481917;
478572; 403755; 476889; 480906; 484626; 490520; 612206; 475172; 403571; 609393; 487498; 475705; 490971; 403433; 480495; 479885; 475769; 486400; 485847;
403721; 403418; 482485; 482486; 403873; 481126; 479324; 488231; 609567; 485063; 609157; 403943; 403912; 403511; 480708; 486834; 478103; 487351; 608234;
609630; 486197; 478755; 611942; 478735; 491952; 479075; 486915; 612212; 489469; 442961; 483312; 491021; 489168; 494007; 490012; 478047; 478279; 478575;
474819; 475596; 474939; 479912; 479911; 611366; 477556; 474585; 613006; 478327; 479960; 488002; 475597; 476731; 609164; 476881; 478089; 606988; 479714;
490883; 553102; 487207; 490630; 610251; 488266; 489372; 478493; 480110; 480930; 607926; 476825; 480784; 485256; 485255; 485828; 479074; 487309; 475035;
487633; 478100; 478952; 611513; 403441; 482861; 608175; 491198; 480348; 489814; 609531; 474667; 475781; 474836; 474838; 481732; 607827; 474869; 607786;
474861; 474862; 481731; 474860; 474860; 609564; 483594; 403170; 486068; 479182; 442946; 442987; 479864; 403897; 490657; 485742; 484434; 485884; 487558;
475260; 607578; 474964; 478450; 481455; 480979; 476466; 480100; 482369; 482370; 482373; 608962; 608958; 486498; 607052; 479668; 442945; 403735; 480811;
476411; 483146; 449023; 489758; 607570; 474630; 475124; 403612; 403612; 480355; 474680; 480726; 479406; 474697; 403979; 611362; 480438; 478854; 476688;
612371; 606789; 403909; 609146; 403975; 480475; 609779; 403547; 477768; 609830; 487737; 481558; 403801; 476216; 487739; 480950; 610255; 442951; 483664;
484067; 490069; 480812; 488516; 491618; 608158; 610316; 607374; 608559; 479967; 479122; 481051; 482839; 403782; 403974; 488126; 403660; 403989; 403870;
403439; 403851; 403785; 489957; 403790; 403985; 612271; 403545; 612582; 403850; 478906; 478905; 611642; 403628; 610823; 484828; 442990; 403623; 403584;
488762; 403796; 480139; 484950; 476836; 478381; 488540; 478432; 483665; 485209; 475554; 484990; 475070; 493994; 475371; 492247; 478800; 475134; 481500;
475633; 476412; 482264; 480015; 486148; 612954; 489210; 489208; 403789; 488429; 486493; 480659; 489905; 489928; 488437; 477956; 403788; 483318; 477210;
608977; 477598; 609255; 476548; 609423; 442952; 484185; 610004; 609429; 484927; 609409; 480522; 479644; 486732; 404022; 490110; 485427; 486733; 485918;
491424; 403758; 487743; 490277; 486639; 485401; 403984; 490441; 484464; 482154; 403811; 403967; 475624; 611325; 611360; 478666; 480446; 491042; 608024;
444857; 486522; 477602; 486513; 490976; 490978; 477601; 492244; 475882; 480034; 477029; 478175; 490391; 479680; 478151; 403830; 491320; 476882; 403616;
450207; 474514; 404021; 483345; 484521; 479193; 442861; 403449; 485544; 611737; 489800; 486363; 478320; 403607; 476186; 480124; 474663; 609006; 403626;
481124; 478781; 479072; 607183; 481712; 484866; 475720; 486728; 484787; 482599; 477886; 612899; 474442; 610286; 481360; 477700; 612436; 612631; 476070;
475456; 480144; 610902; 476196; 487612; 490570; 611884; 484932; 403450; 403451; 488504; 484781; 480488; 475770; 609969; 608715; 480218; 479260; 476160;
489368; 492176; 403537; 403693; 478939; 490041; 478061; 480481; 476262; 476215; 483758; 481826; 403438; 487416; 442978; 611039; 490008; 609832; 607104;
415126; 480229; 477019; 484504; 483732; 479417; 485023; 475461; 477120; 489428; 403733; 403445; 489432; 489429; 403885; 486408; 403763; 403823; 606853;
486335; 480346; 609986; 487114; 494006; 403453; 607904; 494002; 479164; 491924; 484768; 488828; 403663; 403768; 474729; 804478; 804479; 489894; 478230;
804484; 487873; 448784; 483653; 482515; 486309; 609946; 483995; 477232; 403924; 482978; 477024; 485069; 479502; 403808; 479836; 478896; 403614; 476644;
488012; 474410; 480646; 478312; 490344; 611940; 490224; 488447; 481122; 613013; 476725; 487403; 611315; 607700; 480036; 481277; 485677; 608445; 488693;
478032; 474483; 476722; 478869; 477798; 478880; 608738; 480265; 488220; 480021; 487635; 488431; 480618; 477535; 485930; 479552; 486495; 403692; 478813;
474709; 485061; 484920; 442859; 486858; 480291; 484519; 474897; 475450; 475312; 403402; 474484; 488958; 480558; 480559; 606932; 611645; 612773; 477498;
403822; 403784; 480676; 483148; 475083; 475393; 608196; 608289; 487441; 610169; 474762; 612616; 477823; 475354; 482260; 474472; 474984; 611987; 610981;
490404; 484147; 609087; 478987; 485963; 485213; 476822; 477490; 491866; 476749; 478343; 484666; 477129; 482025; 484041; 475351; 478868; 611921; 480878;
490708; 609301; 484670; 448782; 448778; 485203; 483369; 474396; 488746; 491264; 475375; 403476; 609604; 607885; 612814; 492067; 612316; 479839; 609228;
488544; 612021; 609680; 481417; 477596; 480424; 403560; 475618; 476242; 477166; 479149; 485757; 403652; 487221; 491597; 442986; 442860; 442985; 480858;
476574; 476828; 491075; 480868; 482310; 476153; 479875; 610775; 611276; 476371; 475227; 491139; 606872; 488648; 477267; 477597; 480011; 487139; 403621;
403879; 484431; 480422; 476177; 607205; 488192; 488084; 477085; 481772; 483266; 487235; 486443; 484963; 481513; 606755; 487852; 403874; 480048; 484018;
482840; 403426; 476446; 476034; 485586; 485874; 488167; 479473; 482083; 403602; 611266; 489971; 476487; 480914; 611500; 485903; 611484; 478840; 479751;
403907; 480869; 494001; 610047; 482686; 486223; 474915; 479483; 611859; 403659; 475234; 403855; 475235; 489816; 403753; 490360; 403606; 480533; 606861;
478337; 475481; 490021; 480344; 403609; 403557; 487340; 403608; 482873; 484335; 479444; 477374; 478053; 610262; 485672; 491303; 490280; 403486; 489947;
479248; 612559; 612199; 479534; 489082; 489030; 609421; 607309; 479351; 489571; 403556; 479975; 610744; 484774; 490904; 489968; 494005; 609934; 490722;
478686; 484903; 490174; 609091; 478013; 484941; 479577; 474176; 477575; 486758; 478311; 489532; 477746; 474652; 478464; 478347; 611939; 485004; 611397;
476966; 485182; 488241; 479363; 485783; 476104; 490291; 477618; 476855; 608323; 475521; 488121; 483273; 487700; 476402; 403408; 490382; 476867; 475870;
480338; 475132; 490123; 480290; 404305; 475040; 475338; 480537; 475848; 480246; 607466; 474865; 474867; 489749; 478703; 475896; 475980; 476459; 480478;
478522; 478654; 474703; 479671; 477467; 481014; 480256; 480258; 487336; 485097; 403832; 403797; 403589; 403986; 403544; 442942; 403779; 475108; 486600;
610128; 475509; 477263; 490563; 610692; 486717; 607227; 477488; 403662; 477176; 490255; 609852; 475389; 483617; 486664; 479366; 489363; 611060; 478972;
479939; 490781; 486710; 608161; 479210; 404008; 483759; 612683; 403955; 483696; 478088; 484922; 481656; 403568; 490863; 484648; 483420; 475875; 474539;
483225; 476968; 480526; 477045; 486508; 479158; 488774; 476915; 478149; 480854; 477221; 478127; 477090; 477775; 489500; 403972; 479446; 474606; 483729;
484454; 403838; 612532; 476664; 482163; 403494; 479112; 489798; 477308; 478667; 477499; 488584; 488666; 493763; 490310; 475454; 480181; 478328; 484164;
491196; 487339; 475009; 479949; 477912; 475080; 476672; 608703; 476601; 478597; 478356; 475991; 476804; 403677; 474722; 487353; 612448; 491768; 480580;
610499; 475649; 476387; 477933; 476206; 403681; 474599; 476413; 476823; 482963; 491278; 481735; 477074; 403615; 480141; 479915; 612326; 403787; 475852;
490457; 491615; 610295; 480749; 607023; 403676; 480865; 486270; 486839; 403497; 403650; 489598; 489973; 448788; 403981; 480600; 448786; 403522; 491148;
448792; 480603; 403586; 448790; 490356; 487265; 482986; 477945; 482977; 449622; 608210; 480840; 403999; 480080; 403994; 611673; 489508; 608403; 475340;
609693; 612817; 490754; 475730; 478663; 610540; 489066; 403647; 485067; 479487; 490439; 489610; 488542; 482437; 488165; 489640; 403700; 483777; 490939;
477542; 484830; 609304; 404000; 478171; 481490; 491606; 483066; 476335; 476710; 486407; 476640; 486548; 403896; 403896; 479279; 478478; 489880; 476454;
485806; 403559; 476258; 488855; 483042; 490682; 403464; 486507; 488405; 612159; 611255; 608433; 479781; 478471; 488616; 403484; 485864; 403716; 403715;
403475; 481228; 612861; 403944; 478957; 478787; 481082; 403993; 481272; 488449; 481111; 490967; 478845; 490968; 490969; 477258; 482182; 475045; 477240;
485940; 485088; 607840; 483779; 403892; 484403; 609757; 476462; 485402; 477965; 484196; 491950; 477280; 479424; 489846; 480815; 474864; 481740; 611193;
485056; 482771; 477914; 479873; 486303; 610402; 485079; 477966; 481224; 475843; 484064; 484926; 607883; 482555; 403412; 477072; 488989; 488213; 403463;
485941; 488908; 479026; 607720; 403488; 403998; 489932; 488596; 490796; 490387; 481519; 481628; 477039; 608918; 485867; 474355; 487486; 476748; 607489;
403601; 403599; 489365; 610625; 477841; 487731; 403816; 403633; 481289; 403940; 403752; 483343; 476314; 474759; 448807; 482905; 403417; 474771; 482618;
403922; 484006; 403634; 487437; 403566; 477229; 480525; 477044; 403869; 478274; 490397; 489620; 477711; 481598; 484695; 403521; 404019; 612709; 480673;
490867; 480019; 480123; 483844; 489435; 480688; 479883; 485406; 403968; 481275; 489152; 480519; 403627; 480167; 478918; 610636; 478701; 609156; 490339;
474536; 476714; 476100; 479513; 610457; 608118; 492095; 608578; 612742; 477048; 477572; 479010; 478874; 480503; 403574; 403576; 403575; 403595; 480777;
487910; 475085; 486315; 610057; 488646; 612662; 488593; 481089; 403982; 479252; 481590; 474681; 486588; 403802; 494000; 484056; 477991; 476336; 483791;
490848; 480621; 399544; 491867; 488409; 476847; 488806; 486560; 612141; 493997; 609253; 483028; 481623; 476521; 474609; 476447; 488923; 478902; 611171;
477237; 480645; 489818; 475864; 476031; 485384; 489398; 488017; 607083; 485934; 487539; 489584; 475807; 488559; 483104; 608147; 481330; 483900; 479854;
404007; 488246; 481713; 474842; 481714; 474839; 474845; 403848; 489897; 475230; 475295; 608534; 607584; 403767; 610946; 475942; 483724; 489422; 609835;
486718; 474443; 403534; 489667; 480567; 484820; 476431; 485915; 478385; 610872; 479747; 476780; 488790; 490099; 486271; 479955; 483170; 609673; 491034;
612125; 486511; 477892; 481152; 607683; 477392; 477952; 474922; 475451; 489810; 480955; 477749; 489626; 608189; 484867; 484665; 484680; 478350; 476294;
479926; 608508; 475982; 482509; 485022; 480887; 478210; 488423; 609418; 476512; 607962; 478430; 488125; 497070; 475684; 480381; 490006; 609233; 612452;
485548; 486914; 612971; 478152; 487532; 480074; 484019; 610113; 477510; 482093; 611262; 489632; 489684; 489156; 488195; 612984; 611306; 479484; 490434;
475579; 485780; 479580; 403957; 477936; 611694; 475826; 612481; 611961; 479050; 482603; 488471; 482048; 478040; 477052; 403482; 478403; 481708; 490201;
610026; 475702; 475379; 490789; 488103; 477197; 477302; 611468; 480253; 609840; 475024; 488529; 476750; 486851; 475213; 478428; 607751; 609622; 608850;
490926; 442949; 607042; 486251; 479719; 610467; 486680; 488742; 490602; 490050; 479865; 476146; 480450; 487057; 489451; 474636; 483367; 612095; 490370;
479423; 484802; 611442; 489223; 476891; 488649; 480208; 476057; 609496; 480194; 611619; 479307; 486992; 475967; 490831; 491112; 490263; 609100; 476423;
608079; 484959; 490216; 482801; 481657; 489162; 490935; 403683; 479884; 491340; 483299; 403625; 474589; 482101; 481241; 403854; 486973; 489497; 478472;
474822; 606754; 476916; 475756; 485657; 491780; 488481; 610868; 476406; 483640; 478355; 480923; 488651; 475897; 484822; 607954; 480698; 610825; 487158;
485053; 489642; 479648; 490868; 477216; 475266; 480069; 488360; 482715; 482364; 479515; 486242; 477097; 484354; 478950; 474581; 474829; 475817; 474556;
487323; 482822; 486319; 486711; 486876; 474700; 479207; 476713; 481608; 487524; 478541; 478429; 480064; 490907; 403490; 483681; 488653; 612928; 442988;
485643; 474494; 488818; 475217; 478239; 403471; 442964; 487629; 477247; 607247; 612867; 608844; 485038; 608937; 490936; 611211; 476425; 477649; 483625;
476818; 480931; 479744; 403702; 481396; 610835; 474674; 478352; 490032; 481080; 609790; 485043; 483035; 403689; 487343; 491421; 480512; 488438; 479550;
481577; 476689; 608986; 482039; 485031; 478192; 480545; 488179; 490739; 478185; 483838; 485504; 487608; 482460; 474362; 479892; 485674; 484309; 484513;
606955; 609461; 485389; 448789; 608864; 476730; 491231; 475607; 482049; 480818; 483160; 479465; 476864; 480242; 608657; 612549; 611055; 480134; 403473;
487443; 476541; 612823; 483780; 475317; 474986; 484947; 486989; 480588; 489442; 479044; 483728; 485383; 612871; 477974; 611951; 485848; 403594; 488850;
482271; 474445; 480402; 478482; 491064; 612251; 475106; 487316; 475979; 607310; 479684; 490901; 478399; 475767; 476806; 488069; 612760; 476087; 612902;
490410; 481233; 478063; 479177; 477322; 484073; 479773; 445454; 608707; 476418; 481029; 480432; 479127; 476188; 484198; 474851; 490548; 481788; 608154;
475163; 491051; 404018; 477239; 474419; 474837; 481295; 607060; 477901; 475429; 478055; 474452; 607149; 484948; 477147; 608486; 442959; 479672; 486075;
487713; 474675; 481267; 484955; 610925; 608268; 479951; 606961; 474712; 480287; 607929; 486338; 488673; 477067; 480729; 486453; 612152; 478985; 475490;
485192; 479595; 481766; 479479; 480322; 476865; 477133; 477082; 476998; 477834; 608946; 489449; 479600; 606923; 488660; 609600; 606873; 610956; 482813;
477663; 475742; 477351; 479984; 479832; 477202; 479868; 608555; 484646; 475868; 476952; 477805; 486753; 481809; 483310; 609599; 483784; 480677; 490884;
479291; 474670; 476189; 484037; 490874; 488638; 489012; 491294; 474434; 490608; 474579; 403420; 477593; 404017; 491158; 479254; 486403; 609638; 477539;
607896; 474928; 489390; 485782; 476381; 610524; 476419; 610132; 476025; 483821; 478848; 486981; 608367; 477541; 481255; 480709; 481751; 478418; 475049;
612746; 486725; 491230; 487367; 478372; 612792; 485178; 488395; 481266; 610317; 477207; 479535; 483124; 478288; 606908; 486822; 475323; 403553; 480819;
476905; 480595; 484161; 612751; 479317; 492016; 607947; 609080; 488982; 612153; 481270; 484845; 475467; 480913; 477818; 480067; 491499; 481062; 608341;
607103; 485962; 608833; 403519; 403884; 607412; 476643; 489925; 481145; 486549; 477629; 482895; 607659; 479812; 608964; 404006; 476271; 608443; 607003;
480282; 477581; 491909; 484930; 476020; 612797; 609135; 489125; 490154; 481988; 485144; 484046; 482779; 609854; 611189; 476579; 486722; 478974; 478956;
478739; 475943; 476659; 483181; 477138; 476724; 478455; 610496; 477038; 477712; 478507; 607927; 477996; 478474; 480320; 484303; 491068; 609054; 491743;
474513; 489522; 476254; 481418; 611821; 608855; 477639; 481110; 485049; 479731; 488698; 475542; 491160; 477820; 478664; 609794; 486398; 403587; 610120;
488898; 492140; 486762; 476136; 481822; 403720; 480230; 487747; 403502; 475258; 489636; 485774; 477526; 479760; 488971; 477941; 489412; 488210; 478300;
477011; 484518; 479618; 479036; 478007; 475465; 490757; 485809; 482745; 490283; 609221; 476341; 483716; 610239; 476479; 480337; 485858; 484599; 482744;
610101; 477463; 477864; 611540; 484401; 491871; 490419; 482621; 489170; 610117; 608333; 478243; 478677; 490418; 612424; 480077; 490273; 612211; 477146;
477220; 477036; 485905; 484047; 475657; 475740; 608378; 489771; 478872; 490837; 485819; 490149; 480847; 611310; 486308; 486831; 480058; 609473; 490084;
610516; 477093; 479140; 399530; 491709; 487286; 494008; 479132; 607587; 480949; 489094; 480060; 476429; 490046; 481414; 487123; 478564; 485594; 610156;
483347; 481958; 442958; 477567; 611099; 612801; 480447; 486170; 609036; 475086; 487620; 485969; 488407; 482639; 479461; 475330; 609002; 480642; 475913;
483830; 486980; 609101; 483817; 477470; 479020; 475811; 609866; 491839; 485982; 479129; 610942; 476079; 610684; 608355; 611494; 478048; 477190; 609931;
612980; 479734; 612380; 487589; 486744; 477199; 486350; 482850; 482475; 477112; 479551; 612796; 479741; 612601; 609824; 477521; 607562; 479862; 477201; 610442; 491092; 476910;
491076; 481169; 485070; 478533; 487660; 484852; 491012; 480244; 474392; 403518; 607671; 481376; 481813; 478768; 476693; 474422; 475304; 611710; 477885; 484495; 480286; 477609;
474789; 489393; 606935; 477814; 607857; 486615; 486656; 611936; 490771; 476735; 607372; 612116; 478778; 482161; 476283; 608184; 481999; 480569; 478734; 485365; 611852; 609732;
612903; 482645; 613012; 608593; 485597; 482787; 486723; 611023; 475803; 608568; 489942; 488237; 483217; 611401; 606995; 611659
rat subset 1 (b)
rat subset 2 (b)
29275; 29290; 25369; 29316; 171494; 25377; 25292; 25239; 25728; 54226; 292855; 64310; 79121; 360500; 25242; 29657; 25388; 297566; 24223; 24224; 170929; 24888;
60434; 25245; 25181; 287552; 497672; 312705; 192262; 24242; 64202; 114555; 64044; 58918; 25404; 315291; 25109; 25073; 94201; 140908; 114483; 362110; 114851;
83571; 66019; 25301; 29271; 309361; 302290; 54241; 25627; 25707; 84352; 290905; 306628; 363457; 301012; 58954; 81646; 497663; 116549; 84353; 24291; 117184;
64529; 290257; 66021; 24296; 25146; 266729; 25311; 29139; 29167; 25112; 29467; 287765; 114214; 25035; 25633; 24316; 29515; 24329; 24330; 300253; 287986; 24331;
299606; 314436; 25043; 25437; 170915; 313052; 24337; 291703; 29251; 304929; 306761; 315191; 83727; 29558; 84490; 301737; 24366; 363460; 29348; 25022; 260321;
84482; 294515; 287375; 293860; 25661; 314322; 56718; 25019; 361388; 25450; 81659; 498881; 24384; 81716; 25062; 291132; 59075; 81504; 84427; 498416; 24413;
171101; 29468; 296654; 81674; 63864; 171155; 287168; 25734; 288077; 29424; 297893; 300757; 112400; 29560; 192154; 24974; 414789; 294274; 294273; 294269;
29395; 117062; 79239; 25231; 117282; 80846; 117280; 24464; 294254; 25617; 24468; 299331; 497838; 25462; 361384; 313641; 25700; 360415; 288264; 25718; 79438;
25685; 25662; 24484; 360622; 25285; 25641; 289560; 302822; 84351; 24494; 25466; 25704; 140924; 25205; 54258; 29385; 24500; 25325; 116553; 252963; 84472; 54259;
64194; 292892; 296953; 293624; 25467; 170921; 360606; 315346; 308995; 25021; 309684; 25262; 84598; 24514; 25326; 24516; 24517; 24518; 83628; 25468; 25048;
500338; 25110; 288001; 171041; 85245; 300250; 29332; 24530; 300438; 25608; 24536; 83781; 245955; 114903; 65172; 24538; 497722; 299858; 59107; 29357; 25631;
50554; 54267; 29409; 25595; 299996; 24548; 60430; 314856; 304798; 303604; 308106; 85483; 81683; 25480; 60331; 25672; 81686; 81687; 81707; 64466; 81521; 24566;
25483; 310900; 170841; 498982; 296344; 24577; 301059; 299626; 299766; 56781; 337868; 64824; 314910; 288770; 114553; 364018; 25135; 308690; 301458; 25490;
291044; 81735; 311658; 360610; 83619; 81524; 81736; 309452; 25493; 81525; 316241; 25336; 25337; 310738; 25494; 191575; 24600; 81526; 300303; 307982; 25730;
360265; 301430; 305858; 29665; 25506; 288778; 29431; 25737; 363272; 81530; 81745; 361310; 24640; 64303; 81531; 25154; 85243; 24649; 298696; 113983; 24650;
287925; 29692; 25692; 25619; 25096; 25097; 85253; 58971; 296371; 315713; 368070; 29240; 59294; 289758; 304573; 316235; 363633; 299610; 309224; 84024; 29441;
25517; 25664; 497991; 64367; 501204; 24668; 117281; 315648; 117104; 362102; 24674; 29748; 65179; 84400; 50669; 56782; 58826; 25636; 24680; 170538; 29340;
85421; 300445; 25522; 360748; 116590; 50689; 114509; 50658; 25272; 170851; 63880; 24686; 64390; 24691; 286911; 29192; 81751; 29669; 29670; 117263; 117262;
81827; 287984; 296651; 25614; 366275; 29222; 24924; 116689; 25622; 24699; 315595; 307259; 25270; 81755; 64633; 363875; 366957; 309151; 361381; 298012; 362787;
24703; 295347; 313644; 24708; 81758; 25703; 140936; 305584; 309165; 365510; 89809; 116468; 288414; 288003; 304528; 29480; 24717; 291713; 499652; 287524;
59102; 81763; 290641; 367874; 29288; 81771; 83840; 361568; 25271; 170546; 295214; 83576; 24615; 81778; 445415; 295213; 60352; 246074; 24768; 25615; 83841;
25544; 25651; 307947; 252855; 117259; 64667; 85385; 289734; 171379; 361825; 301020; 363002; 113893; 29219; 313845; 24790; 24791; 366126; 83805; 78968; 300095;
501099; 498351; 361811; 304833; 29435; 25124; 25125; 367264; 24918; 25126; 362896; 29149; 116470; 308472; 25155; 317256; 25123; 294018; 288533; 373541;
313121; 24811; 24812; 25217; 117526; 24816; 84382; 24817; 171046; 25720; 366964; 291571; 301965; 301285; 81808; 59086; 84574; 81809; 116487; 29591; 81810;
316742; 56083; 83580; 81812; 304109; 25358; 25628; 292994; 362533; 313494; 24835; 25625; 497808; 29248; 64550; 24842; 296099; 362675; 500959; 500450; 362862;
311786; 362788; 311245; 295230; 60395; 292925; 25360; 25274; 287208; 24856; 316531; 498736; 116484; 298706; 298317; 25573; 361585; 301442; 25574; 314981;
304577; 498306; 305679; 116643; 24873; 24874; 54319; 25696; 29169; 116669; 117538; 290805; 24883; 85252; 84495; 309995; 363247; 24919; 56011; 29753; 56010;
25576; 25578; 353227; 58820; 313666; 362558; 295238; 29619; 64158; 117022; 360471; 291133; 294250; 246760; 296634; 80848; 309072; 25445; 25379; 50719; 306347;
84584; 304157; 363445; 288471; 89816; 360908; 89812; 117274; 497801; 301555; 501104; 307766; 83507; 309295; 85246; 498183; 79431; 300772; 310806; 94193;
171137; 29647; 311130; 117516; 56042; 246334; 313929; 29419; 252971; 58945; 29376; 298700; 311371; 364052; 246756; 303068; 306886; 81830; 362491; 84607;
117279; 65154; 84578; 301164; 114559; 84019; 84005; 192234; 296302; 295052; 362249; 361765; 361420; 360640; 89829; 368002; 360600; 282827; 83509; 83422;
360481; 311021; 64520; 305000; 310635; 317399; 83631; 361724; 64896; 65040; 79434; 64193; 312897; 287609; 114505; 315769; 363603; 116501; 59114; 497738;
300057; 312849; 114523; 309565; 300367; 499022; 29702; 84591; 286936; 116457; 315740; 79252; 293524; 432361; 83611; 25414; 362251; 500131; 116598; 54299;
360801; 303285; 363984; 309595; 116673; 362294; 300146; 303002; 266601; 292087; 117519; 113970; 304017; 305240; 408200; 65035; 84485; 300084; 114485; 171138;
299180; 497768; 114552; 64547; 363333; 362293; 308384; 58852; 501546; 54227; 81732; 315745; 293852; 362686; 116698; 362501; 89827; 319110; 64845; 116633;
287155; 117026; 500929; 296554; 83427; 83614; 64456; 364382; 192260; 289810; 315139; 117542; 291150; 295686; 64185; 363113; 363026; 83724; 303396; 60571;
81823; 315133; 192218; 307189; 29374; 299809; 501203; 64015; 117268; 29428; 288305; 293144; 64459; 288444; 63885; 291948; 83516; 291005; 307474; 84014;
301226; 298068; 311621; 192277; 25577; 304538; 312916; 361319; 315333; 171097; 246240; 317366; 297930; 291999; 114562; 266709; 289437; 363140; 293619;
315000; 171159; 301014; 308335; 365355; 25482; 29372; 308003; 304286; 360557; 361502; 313717; 84398; 361196; 294864; 298604; 290704; 310960; 314894; 499250;
116464; 363544; 293455; 293410; 290447; 266806; 316228; 308267; 313819; 296588; 293476; 306264; 309362; 303024; 499010; 289231; 65153; 84551; 300266; 94165;
116486; 362201; 29392; 246146; 171071; 293344; 85242; 300656; 310392; 499882; 305475; 366960; 300115; 296639; 289508; 311796; 83718; 363067; 288124; 361474;
171574; 289521; 287544; 300066; 246314; 309374; 290556; 360681; 259275; 171350; 81511; 500870; 60382; 304920; 364624; 289662; 292762; 317612; 361767; 289757;
83709; 299827; 306492; 140938; 80843; 314941; 170912; 317396; 499670; 84357; 65199; 295419; 299976; 315097; 290541; 306655; 314961; 312710; 116479; 114022;
295961; 314759; 295531; 29470; 297597; 301013; 499298; 366975; 499171; 296731; 361769; 497961; 360734; 297695; 292041; 80897; 266688; 500377; 311926; 114858;
94272; 63848; 25309; 304580; 300944; 304714; 499884; 170837; 292759; 292064; 315732; 361273; 307545; 309144; 64531; 287633; 307126; 363334; 171444; 57341;
299121; 393088; 116741; 81918; 114518; 361666; 296562; 65208; 308441; 64042; 300075; 298646; 311299; 310632; 360866; 362224; 313876; 116478; 305230; 312246; 246273; 500954; 66026;
497972; 287146; 308173; 291279; 291369; 306764; 64345; 302963; 497835; 294520; 113912; 299933; 500872; 303317; 287830; 310352; 286761; 499288; 498281; 311703; 502898; 311567; 360968;
362545; 83582; 363292; 362819; 307351; 361054; 84686; 287474; 266670; 300043; 315691; 303517; 85265; 114481; 308516; 311055; 288916; 498130; 307585; 313842; 297822; 312947; 308023;
298689; 307850; 246174; 297705; 116674; 298705; 498769; 367160; 286965; 291129; 56763; 60564; 317715; 295620; 295171; 315388; 316071; 500110; 500847; 365895; 293497; 364681;
316758; 291081
rat subset 3 (b)
24153; 116631; 24795; 57300; 497803; 311860; 65029; 304883; 501072; 25287; 25014; 79123; 50681; 24161; 29437; 25365; 81822; 29275; 303742; 63836; 361925;
81634; 291245; 24165; 29650; 24170; 298199; 24172; 360354; 25026; 29290; 25369; 29316; 25370; 25591; 29412; 24176; 25645; 25238; 25372; 308578; 81637; 29663;
140670; 171494; 29466; 24177; 58968; 81722; 29146; 24179; 81638; 24182; 25690; 25373; 24185; 25233; 25374; 24186; 363516; 84356; 24189; 24190; 24191; 266802;
287454; 25290; 81639; 64621; 25586; 301585; 24197; 25377; 361367; 60668; 499694; 499694; 306570; 81641; 85333; 56611; 25673; 79125; 78963; 500761; 83589;
83610; 29722; 305338; 24205; 29339; 79116; 60371; 78971; 63879; 502317; 64312; 25649; 54225; 25383; 25292; 292697; 25239; 25728; 54226; 292855; 246097; 25385;
25240; 65133; 24208; 81002; 29183; 64310; 140940; 79120; 303559; 79117; 79121; 29221; 29215; 117273; 64373; 295342; 308875; 360678; 362456; 360500; 25242;
29657; 25387; 25388; 315222; 84431; 24209; 24210; 29403; 25612; 304917; 307829; 315305; 25389; 79255; 300711; 116665; 24211; 24213; 171028; 29132; 24214;
312488; 297566; 29757; 24221; 25107; 29462; 25108; 24223; 304127; 64639; 297994; 116502; 64557; 24887; 65036; 58919; 24224; 170929; 24888; 60434; 292700;
303836; 309696; 25245; 25394; 25295; 25181; 64625; 114496; 309871; 364403; 308755; 287552; 116599; 83470; 29373; 85272; 367786; 306012; 140923; 29884; 296321;
497672; 114486; 29344; 298765; 25246; 64022; 84410; 29618; 29681; 298566; 312705; 192262; 24231; 362119; 117517; 313421; 497751; 310218; 54231; 298657;
291819; 313495; 363085; 25398; 24239; 25678; 25399; 50688; 116600; 25297; 116506; 24242; 50663; 64202; 25400; 24246; 171140; 81715; 316010; 29144; 29153;
29154; 25403; 25166; 64314; 25402; 114555; 83584; 64026; 64044; 58918; 29209; 24248; 25404; 367218; 50662; 362489; 156726; 500985; 29345; 304078; 25706;
114494; 114839; 64033; 25729; 25405; 29157; 315291; 288620; 25109; 497761; 24932; 54236; 303747; 24930; 60350; 500318; 25660; 25408; 497809; 25073; 171369;
84349; 25406; 29364; 245962; 29186; 287435; 29187; 25599; 362638; 54237; 360621; 64515; 307511; 64465; 83502; 497718; 116777; 307618; 310174; 94201; 140908;
114483; 362110; 114851; 83571; 246060; 25164; 54238; 289993; 81925; 66019; 24252; 24253; 25410; 25301; 291863; 29271; 366624; 25423; 24255; 307092; 140583;
24258; 81645; 24260; 25590; 309361; 360785; 302290; 308015; 25688; 29232; 84360; 25749; 295586; 29233; 24854; 406864; 301434; 365842; 65160; 83800; 54241;
25627; 57301; 60463; 117027; 171054; 117029; 59264; 58936; 65204; 25707; 117258; 29393; 84352; 25412; 84032; 290905; 363457; 85490; 85250; 294337; 361821;
301012; 305104; 362584; 362285; 25654; 294027; 24267; 114023; 58954; 24271; 113936; 289395; 25061; 29563; 81646; 497663; 58959; 338401; 54245; 25419; 81649;
78965; 307403; 116630; 497822; 25610; 298518; 315707; 113927; 64462; 58822; 116549; 307641; 114122; 81651; 29317; 64032; 24962; 307505; 84353; 311163; 114028;
24291; 117184; 64529; 171293; 25424; 290257; 25425; 29175; 50654; 116639; 64001; 79129; 66021; 300047; 24296; 24297; 171352; 361523; 29296; 293985; 246070;
29277; 25086; 171352; 498198; 24307; 25428; 29680; 353498; 24294; 25146; 25147; 24298; 301517; 25427; 266729; 79128; 64036; 64322; 306722; 140926; 25699;
25311; 29139; 24310; 29167; 64470; 311186; 291283; 291283; 25112; 29467; 287765; 304859; 300710; 363256; 114214; 298298; 24312; 25035; 24314; 81654; 298942;
25252; 497670; 287638; 303469; 25312; 140694; 25633; 25751; 84350; 294051; 25253; 24316; 24318; 29238; 314616; 29515; 307563; 83514; 25254; 25433; 114856;
311406; 60587; 171109; 300980; 497778; 83721; 399489; 307794; 116651; 287114; 94204; 361921; 302424; 116744; 24323; 50672; 171361; 293725; 29565; 25313;
24329; 24330; 114090; 25148; 25129; 302697; 54318; 64514; 299027; 366492; 287436; 300253; 117045; 116636; 361845; 287986; 287444; 24331; 299606; 497806;
363854; 302811; 366142; 314436; 304786; 25043; 25437; 60465; 24334; 63997; 170915; 29452; 313052; 313633; 287989; 25315; 24335; 24336; 289352; 312812; 24337;
29496; 59323; 291703; 301382; 306274; 170909; 498013; 292747; 25149; 24356; 304063; 303828; 303687; 289752; 303547; 312299; 29251; 25439; 116677; 25584;
304929; 29243; 290757; 306761; 29347; 25598; 25288; 114024; 113976; 50646; 192203; 315191; 83727; 171075; 295279; 498277; 289211; 304966; 29558; 83791; 84490;
301737; 361597; 24366; 363460; 54250; 29348; 84489; 25022; 25114; 24367; 63839; 25639; 293702; 260321; 361810; 192274; 58921; 84482; 294515; 310586; 315532;
287375; 293890; 293860; 54251; 246245; 246247; 246248; 25661; 314322; 308411; 292409; 499335; 56718; 25449; 60349; 25319; 83826; 25320; 317385; 25150; 24377;
29573; 25019; 361388; 363735; 29708; 25450; 29694; 81659; 29423; 498881; 499156; 25172; 25159; 29300; 24384; 24952; 29244; 171128; 303539; 29152; 24387; 27100;
81716; 156275; 25235; 24392; 25655; 29585; 316033; 140589; 282838; 117533; 81663; 89788; 50664; 60429; 24400; 60449; 64528; 24401; 25721; 313017; 25259; 25062;
292804; 291132; 84475; 59077; 59075; 299618; 303248; 81670; 24404; 29328; 81504; 84427; 24408; 24409; 24413; 114105; 171551; 171101; 29468; 84027; 296654;
116686; 24422; 501110; 24424; 57298; 64352; 29438; 25260; 366547; 83830; 83828; 81673; 303918; 306516; 316123; 81674; 361580; 294693; 294236; 353256; 299347;
25202; 266708; 171528; 24437; 24438; 500987; 58940; 63864; 171155; 25632; 25632; 24440; 361619; 293267; 502359; 94164; 501681; 287168; 25734; 288077; 29424;
297893; 84577; 300757; 294673; 155012; 29199; 289057; 24446; 112400; 79237; 303310; 29560; 192154; 289623; 500155; 25059; 24974; 414790; 414789; 294274;
294273; 24984; 365542; 24986; 309621; 294269; 294270; 294270; 294228; 309600; 24973; 25459; 29395; 25675; 117062; 24451; 79239; 79240; 25098; 25099; 25100;
25735; 25231; 362361; 83498; 79256; 117282; 80846; 117280; 368057; 497985; 287647; 24459; 368178; 24464; 24465; 502373; 499420; 60421; 29577; 360348; 25116;
25117; 25322; 79244; 65028; 294254; 294254; 60460; 266759; 25617; 24468; 291671; 24471; 161476; 299331; 297852; 301252; 497838; 25462; 361384; 313641; 498411;
24476; 24477; 25464; 360647; 25261; 25587; 25585; 25700; 89784; 304988; 360415; 298205; 288264; 24481; 25712; 116465; 360697; 58845; 24482; 25718; 24483;
25151; 79438; 25685; 25662; 24484; 360622; 25285; 25641; 289560; 83476; 297767; 302822; 84351; 24493; 24494; 25466; 60582; 116562; 25704; 140924; 287287;
25084; 24497; 24498; 24499; 25205; 294797; 54258; 29385; 24500; 25325; 117539; 171333; 116553; 252963; 25670; 116996; 29197; 310612; 84472; 170922; 306626;
24504; 245920; 24506; 54259; 65038; 64194; 24954; 299872; 298439; 363520; 114517; 64831; 24508; 290749; 292892; 291100; 296953; 364081; 293624; 25467; 64444;
25118; 170921; 360606; 311144; 315346; 308995; 25021; 296456; 24511; 309684; 25724; 305506; 25713; 311422; 25262; 60583; 84598; 24514; 25326; 24516; 24517;
24518; 81679; 83628; 292028; 24520; 25468; 29731; 25469; 64358; 25736; 65180; 65195; 25471; 65198; 25472; 83535; 83731; 54263; 65206; 84020; 64030; 24594;
25048; 500338; 25110; 288001; 171041; 24917; 85245; 24525; 300250; 300242; 360625; 303530; 294853; 360626; 50687; 309368; 298941; 117036; 29236; 29332; 24530;
313050; 94106; 170496; 500965; 300438; 25608; 24536; 56646; 171134; 83781; 245955; 25474; 116641; 25476; 60584; 360304; 290907; 114903; 65172; 29524; 24538;
25330; 116666; 60374; 497722; 24539; 299858; 116565; 25008; 361795; 290623; 59107; 297604; 301034; 81682; 81515; 501502; 25211; 494343; 171577; 312689;
294446; 362391; 297176; 25671; 29357; 25631; 50554; 367100; 81516; 54267; 29409; 246136; 361378; 29253; 25750; 25595; 367171; 171347; 60661; 59108; 291439;
24548; 78967; 317598; 60430; 29333; 81517; 314856; 304798; 81518; 58923; 116667; 303604; 308106; 365057; 29417; 25684; 313662; 25332; 25333; 85483; 54252;
81683; 246759; 25480; 60331; 291234; 497834; 308463; 309168; 315606; 301119; 25672; 24590; 300339; 81686; 171045; 25335; 63849; 81687; 117061; 25481; 171052;
81707; 304608; 498194; 266713; 303413; 291327; 64046; 24565; 64466; 81709; 499505; 81521; 24566; 81710; 25483; 26195; 26198; 117260; 362657; 26202; 310900;
170841; 498982; 296344; 24577; 298894; 301059; 299626; 360543; 29557; 56781; 24585; 299509; 65261; 25017; 81523; 337868; 29148; 64824; 314910; 288770; 50594;
85482; 114553; 364018; 500904; 300955; 25135; 308690; 291660; 315167; 299643; 311029; 301427; 294964; 293991; 301458; 25490; 291044; 81735; 29458; 24592;
25744; 311658; 25492; 366998; 360610; 83619; 29227; 29228; 81524; 81736; 309452; 25493; 81525; 316241; 29508; 25336; 25337; 310738; 300092; 25494; 191575;
83782; 291084; 314969; 501589; 24599; 24600; 25496; 29492; 81526; 290029; 300303; 24602; 114593; 116564; 25339; 307982; 25499; 312195; 497735; 58813; 25730;
29541; 59109; 25054; 29613; 360265; 366274; 308870; 81738; 302552; 25502; 81528; 171116; 25341; 25601; 313479; 301430; 289747; 25611; 305858; 25504; 25342;
29665; 29597; 25506; 288778; 497733; 117254; 24617; 60325; 29431; 29432; 29433; 24618; 56816; 360464; 64513; 293992; 114502; 29700; 500242; 65051; 362282;
81740; 25604; 25737; 25204; 25121; 50678; 24627; 25266; 25267; 497724; 306640; 117098; 289950; 116551; 81530; 296849; 89813; 85496; 81745; 287526; 308321;
500006; 116718; 361310; 300257; 24640; 117276; 64303; 81531; 25154; 24891; 25344; 24648; 116589; 288937; 361241; 170911; 85243; 24649; 298947; 64161; 298696;
113983; 24650; 498328; 25630; 29692; 24653; 25157; 25692; 25619; 29322; 24655; 25738; 25096; 25097; 64204; 85253; 287527; 25515; 58971; 302562; 117540; 296371;
315713; 494322; 288479; 368070; 29240; 59294; 289758; 304573; 316235; 363633; 299610; 309224; 24664; 84024; 296851; 312086; 29441; 25517; 171068; 25664;
497991; 64367; 501204; 302934; 24666; 24668; 360258; 313030; 24672; 24673; 117281; 315648; 117104; 60660; 117256; 363122; 362102; 312754; 24674; 171366;
24675; 29748; 65179; 171121; 29471; 50669; 56782; 58826; 65248; 78975; 25636; 293508; 114906; 24678; 29699; 24680; 25023; 170538; 29340; 81749; 84006; 29355;
207122; 85421; 85420; 300445; 25522; 360748; 116590; 50689; 114509; 50658; 25272; 170851; 58960; 29568; 363855; 63880; 308845; 24683; 24684; 24686; 25268;
305066; 24688; 24689; 64390; 25052; 24691; 286911; 24718; 288291; 29245; 65164; 314615; 29192; 81751; 364993; 29668; 29669; 29670; 29671; 29672; 29673; 29674;
94198; 29675; 29676; 58854; 29425; 29666; 85492; 24968; 24967; 291983; 117263; 25581; 29677; 117262; 81827; 289990; 287984; 83499; 296651; 307821; 161475;
29630; 29614; 58949; 29497; 89830; 50557; 81752; 84023; 24694; 497767; 29527; 56813; 25614; 366275; 315265; 29222; 24924; 24697; 117063; 500465; 116689;
246781; 25622; 360370; 24699; 114767; 360406; 497775; 29616; 50677; 497784; 294800; 315595; 307259; 25270; 299196; 312703; 81755; 25532; 64633; 293694; 25533;
363875; 366957; 309151; 310034; 361381; 298012; 499870; 362787; 24703; 84600; 81757; 294429; 295347; 313644; 24705; 24706; 25676; 192213; 24708; 313048;
83712; 81758; 25056; 25703; 140936; 25534; 315655; 305584; 309165; 365510; 24715; 81759; 83618; 24716; 89809; 116468; 288414; 288003; 304528; 84583; 29480;
24717; 192264; 359726; 361390; 81762; 300807; 309288; 287524; 59102; 300079; 81763; 297755; 290641; 287417; 367874; 57809; 140661; 140662; 140654; 29288;
29304; 81771; 117269; 501560; 83840; 81772; 497882; 65139; 367324; 81777; 361568; 365320; 362720; 25271; 140585; 498783; 170546; 295214; 83576; 24615; 445415;
295213; 25742; 365245; 60352; 362890; 302642; 288663; 246074; 25665; 25122; 24768; 287562; 116637; 81780; 287561; 29397; 117518; 29538; 298006; 117551;
305236; 171371; 89808; 25216; 25615; 83841; 24772; 298596; 501550; 25544; 29259; 25651; 307947; 252855; 494445; 362612; 362264; 362687; 117259; 24773; 29517;
64667; 24775; 85385; 24781; 24778; 25351; 65197; 29484; 50567; 24779; 289734; 114613; 24898; 29464; 24782; 24784; 497723; 29723; 81826; 84386; 171379; 361825;
301020; 363002; 64301; 64301; 499417; 113893; 29219; 64347; 292729; 24786; 24787; 25352; 313845; 85384; 24790; 367846; 24791; 366126; 266602; 24796; 25353;
310575; 83805; 24950; 64677; 78968; 300095; 501099; 498351; 304833; 81783; 29435; 24797; 497823; 25124; 288774; 25125; 367264; 24918; 25126; 362896; 84496;
360268; 65189; 311622; 261730; 314621; 29149; 116470; 65134; 25558; 83783; 292915; 287608; 308472; 25559; 361256; 25155; 24949; 57027; 317256; 25123; 294018;
288533; 373541; 313121; 24811; 24812; 25217; 117526; 24816; 24886; 64525; 81807; 24817; 25640; 171046; 25705; 366964; 310661; 24818; 29338; 29362; 64523;
301965; 29460; 83474; 306862; 301285; 362280; 81808; 113984; 361178; 25563; 24827; 59086; 84574; 81809; 25717; 289316; 116487; 29591; 81810; 316742; 56083;
83580; 64517; 81811; 81812; 24831; 24832; 312510; 361655; 304109; 116510; 29543; 25358; 25628; 292994; 362533; 313494; 310553; 29260; 25566; 24835; 25625;
497808; 29388; 29248; 64550; 361100; 24842; 296099; 362675; 500959; 500450; 54314; 362862; 311786; 362788; 311245; 295230; 60395; 89823; 60445; 24855; 292925;
25360; 25274; 287208; 303536; 25571; 24856; 316531; 498736; 85489; 89814; 116484; 58819; 298706; 29261; 301000; 298317; 361831; 81920; 498449; 64631; 25573;
363836; 361585; 301442; 25574; 24860; 54315; 25708; 501907; 83808; 286954; 50555; 314981; 304577; 58851; 289801; 83586; 81817; 498306; 25361; 305679; 116643;
83529; 24873; 83785; 24874; 54319; 81818; 25696; 293735; 362779; 24877; 29169; 116669; 317371; 117538; 308937; 305455; 24881; 114487; 290805; 24883; 497811;
298074; 312560; 85252; 84495; 309995; 363247; 24919; 56011; 29753; 56010; 25576; 25578; 79426; 361627; 353227; 58820; 313666; 311764; 362558; 117180; 29619;
78947; 64158; 117022; 60628; 360471; 29448; 291133; 294250; 94342; 114612; 361796; 301265; 246760; 286926; 296911; 58853; 296634; 80848; 309072; 117505;
25445; 64624; 297593; 299810; 84017; 25379; 50719; 114124; 306347; 292683; 84584; 304157; 363445; 363518; 288471; 89816; 83522; 79257; 360908; 304323; 89812;
117274; 266770; 497801; 314862; 361181; 301555; 361258; 313994; 501104; 307766; 288597; 26295; 83507; 29738; 309295; 85246; 266689; 299802; 79431; 300772;
310806; 94193; 500526; 171137; 29647; 171445; 311130; 117516; 65137; 64369; 56042; 246334; 171516; 313929; 29419; 252971; 58945; 29376; 292148; 298700;
499619; 311371; 364033; 292483; 364052; 288701; 84497; 246756; 500592; 89842; 303068; 306886; 117271; 301133; 287437; 246775; 57025; 361170; 114101; 366518;
81830; 362491; 266610; 364420; 310682; 500715; 114087; 361598; 84607; 117279; 65154; 84578; 301164; 84385; 85238; 294235; 114559; 84019; 117019; 171145;
84005; 192234; 296302; 295052; 302671; 290215; 306332; 294568; 501164; 314633; 361765; 29430; 361420; 360640; 89829; 296566; 368002; 282827; 312790; 308761;
83509; 317163; 287101; 360481; 311021; 64520; 362485; 54190; 363134; 361321; 56084; 171148; 305000; 29606; 498609; 25556; 310132; 317399; 83631; 58857;
361724; 64896; 65040; 79434; 64193; 312897; 295704; 314384; 287609; 116693; 116692; 116691; 29415; 502603; 286920; 114505; 363603; 287739; 29282; 116501;
296706; 59114; 246253; 60391; 300057; 366962; 361743; 25429; 114523; 312382; 309565; 499022; 290364; 29702; 117186; 286936; 363275; 365601; 60575; 116457;
315740; 84469; 66015; 79252; 64198; 29455; 59103; 432361; 83611; 298655; 25414; 113960; 362251; 500131; 94167; 311047; 116598; 312331; 54299; 360801; 408210;
363984; 500616; 309595; 308306; 116673; 362294; 303002; 304582; 497795; 497720; 266601; 292087; 117519; 362510; 304017; 305240; 304914; 287773; 50622;
170582; 65035; 60351; 84485; 300084; 64535; 114485; 362189; 171138; 29414; 170588; 114552; 64547; 300219; 363333; 362293; 308384; 114114; 58852; 84388;
501546; 54227; 81732; 289820; 171497; 64012; 315745; 287069; 293852; 362989; 170842; 362686; 116698; 25029; 362501; 89827; 301056; 78969; 316130; 319110;
294961; 64845; 294981; 306141; 315756; 58965; 313564; 116633; 287155; 315958; 84026; 292756; 117026; 65276; 308899; 500929; 296554; 58834; 299923; 83427;
83614; 363014; 64456; 364382; 170908; 289810; 308416; 117542; 313702; 291150; 295686; 497739; 64185; 363113; 363026; 83724; 303396; 60571; 81823; 192218;
308939; 362214; 307189; 362248; 313139; 84395; 29374; 299809; 64353; 303477; 310645; 64015; 117268; 362160; 29428; 83726; 303634; 288305; 297336; 293144;
308069; 310344; 64459; 25491; 315159; 83722; 288444; 63885; 361652; 362505; 64537; 291948; 362782; 83516; 307366; 291005; 361798; 84014; 301226; 64371; 63867;
360609; 298068; 311621; 362891; 192277; 362859; 25577; 312916; 64370; 361319; 299783; 315333; 315461; 296197; 246240; 317366; 291999; 304135; 315121; 114562;
266709; 362960; 289437; 362930; 313323; 314126; 363140; 114212; 291526; 63995; 300974; 296709; 302669; 64014; 290692; 286904; 140860; 117287; 361813; 58974;
308335; 302248; 365355; 362229; 25482; 282840; 29372; 308003; 304286; 360557; 313717; 84398; 304962; 293897; 294864; 296126; 290704; 310960; 499250; 116464;
363544; 293455; 192226; 291209; 290447; 294007; 266806; 316228; 360658; 316640; 293721; 296588; 309338; 303024; 117243; 499010; 314465; 289231; 309757;
65153; 84016; 84551; 85428; 155437; 300266; 367253; 94165; 302969; 116486; 362201; 497836; 29392; 113910; 246146; 171071; 293344; 85242; 300656; 310392;
363228; 499882; 305475; 366960; 300115; 296639; 288227; 362900; 289508; 362437; 311796; 83718; 363067; 288124; 308843; 113961; 361474; 300066; 246314;
362247; 246303; 295338; 66017; 309374; 113906; 360681; 361057; 114512; 259275; 171350; 117089; 81511; 500870; 307927; 27137; 500906; 60382; 64031; 304920;
289662; 317612; 361767; 289757; 171144; 140915; 58978; 313974; 83527; 316013; 282817; 299827; 315298; 300193; 306492; 140938; 25435; 117277; 80843; 365583;
54282; 83799; 170912; 317396; 304670; 84357; 65199; 65023; 288378; 295419; 287659; 299976; 289388; 85431; 292279; 296981; 315097; 290541; 306655; 312710;
116479; 114022; 291137; 295961; 314759; 365872; 362228; 362850; 360915; 288985; 252902; 297597; 161452; 307133; 299117; 301013; 317468; 499214; 29465;
246302; 499298; 300126; 366975; 155140; 362827; 499171; 296731; 497961; 94340; 360734; 297695; 116593; 59301; 292041; 80897; 317589; 500377; 311926; 94272;
171554; 299256; 245975; 25309; 313729; 311446; 304580; 304294; 308913; 24861; 54705; 315650; 83836; 499884; 170837; 292759; 292064; 308775; 361273; 305419;
64531; 114246; 60462; 309434; 308365; 293886; 171444; 245956; 292788; 299121; 296315; 114518; 116469; 288753; 361666; 246250; 308589; 116493; 310633; 314721;
64042; 300075; 298646; 311299; 288025; 310632; 499099; 360866; 171358; 362224; 499932; 301073; 171373; 498999; 497729; 116478; 287871; 246311; 361495;
363235; 500711; 246273; 503122; 310820; 298784; 302668; 500954; 66026; 296872; 368128; 306868; 287146; 308173; 307514; 291279; 291912; 64133; 305392; 291369;
306764; 64345; 302963; 312516; 259171; 171415; 497835; 363273; 303702; 294520; 64803; 287962; 64457; 113912; 306811; 361848; 362789; 171384; 299933; 500872;
311731; 498938; 497827; 313588; 367083; 303317; 287830; 310352; 286761; 311865; 310399; 282845; 292995; 295323; 316561; 302511; 79130; 259243; 80773; 85251;
116745; 311545; 499288; 361315; 296278; 498281; 289582; 311703; 300802; 502898; 311567; 360968; 290833; 362596; 502531; 362545; 363760; 362393; 360491; 304547; 363292; 497867;
362819; 362245; 287624; 361054; 84686; 366891; 287474; 266670; 300043; 315691; 303517; 85265; 114481; 308516; 360992; 316231; 170739; 288916; 299828; 291278; 307585; 312166; 297822;
312947; 308457; 497816; 140638; 298689; 309465; 300260; 246174; 297705; 116674; 301123; 498769; 289565; 498995; 286965; 140734; 291129; 60564; 317715; 363328; 296285; 295620; 295171;
315388; 316071; 500110; 171018; 499702; 365895; 293497; 364681; 316758; 295341; 362278
mouse subset 1 (b)
232345; 104112; 11486; 11522; 11607; 11657; 11674; 12257; 12359; 12608; 12613; 13076; 68444; 13170; 18104; 13653; 13806; 382044; 14381; 103988; 14718; 14775;
14862; 110006; 14958; 15130; 15368; 15481; 16000; 16009; 16175; 16476; 16477; 21408; 16956; 24059; 17194; 17748; 17869; 17883; 17380; 18053; 18160; 18263;
18787; 18639; 18669; 18770; 19052; 19053; 19246; 19401; 19699; 20277; 20296; 20971; 20493; 20535; 545845; 11611; 20716; 21354; 234724; 22059; 12759; 22139;
100559; 22346; 20865; 16644; 225845; 78925; 107869; 15007; 109754; 231691; 18703; 114228; 52118; 16190; 13106; 18806; 15376; 19733; 54140; 13197; 15483; 20848;
17919; 17975; 13074; 239134; 17105; 15379; 14600; 13482; 14261; 14431; 15901; 19299; 14081; 11812; 12097; 18673; 21835; 11810; 11910; 12367; 12450; 13121;
13122; 14854; 15894; 16493; 18027; 18035; 18148; 18553; 268373; 19261; 20103; 20928; 19181; 15903; 11647; 15902; 16149; 18416; 27053; 21937; 13419; 14377;
14225; 11807; 11931; 15357; 16006; 11622; 13039; 19662; 11816; 18538; 109731; 70025; 26961; 27062; 13179; 22154; 12408; 18028; 20775; 26371; 16785; 17224;
12631; 72082; 545679; 13097; 11861; 11541; 13008; 12192; 12406; 12842; 19173; 224105; 11459; 18984; 19243; 13198; 16803; 13424; 13867; 19205; 20498; 20516;
13498; 18601; 16886; 19139; 13629; 15382; 14137; 14622; 12227; 208715; 11764; 26440; 26442; 26444; 26446; 19182; 20227; 12261; 17330; 11938; 12289; 12012;
16533; 14613; 17254; 20522; 238055; 11820; 12350; 12352; 18227; 13665; 22350; 54399; 64385; 58809; 217869; 80906; 11491; 50493; 236539; 19172; 12443; 56455;
23849; 54375; 109900; 55989; 16905; 15469; 13043; 18432; 12727; 268756; 56183; 109672; 110095; 217119; 231070; 73078; 12317; 434345; 56720; 56362; 18242;
11852; 26378; 209354; 223775; 19943; 12015; 52551; 94224; 67443; 232910; 243187; 64705; 20116; 56722; 20888; 11783; 20787; 11792; 11843; 13057; 15160; 15486;
22695; 16005; 15893; 14825; 268860; 11608; 432454; 16790; 27421; 108147; 76282; 68705; 104776; 207728; 107650; 432861; 19921; 19934; 67437; 21847; 22019;
22352; 11461; 14694; 15384; 19185; 242939; 18024; 18506; 16854; 20887; 432878; 110196; 56449; 386649; 193034; 18979; 56185; 12825; 18606; 26874; 216233; 18631;
56794; 109042; 319554; 68278; 11758; 19170; 58994; 18412; 56312; 394431; 74205; 226751; 192166; 12909; 12539; 53817; 50798; 14873; 12575; 19252; 233187; 14664;
21857; 228026; 16198; 74325; 13685; 67603; 20970; 19273; 70789; 14782; 19158; 18705; 16511; 67397; 432502; 12557; 75423; 71911; 12492; 23957; 13595; 16601;
11966; 68961; 66234; 11842; 16819; 18483; 28250; 170483; 170639; 17872; 20504; 240672; 29811; 234671; 74147; 108067; 56523; 68090; 107975; 105349; 226105;
56448; 81601; 67103; 52858; 171210; 546723; 15945; 20249; 228775; 230967; 382051; 71514; 93732; 72433; 217166; 56388; 67952; 20730; 13118; 280662; 14667;
109815; 13094; 71773; 107515; 98238; 110172; 21762; 245841; 15505; 231872; 27979; 66437; 215210; 101966; 226470; 74106; 14373; 246177; 67939; 68263; 30877;
76808; 545631; 102022; 243867; 13089; 54131; 13626; 433564; 54123; 225887; 545292; 15040; 15937; 14999; 22213; 71733; 52521; 16668; 12258; 98221; 229003;
66865; 66489; 93760; 76238; 11520; 69902; 12401; 104130; 15519; 70396; 216453; 27367; 192653; 76491; 15516; 19076; 70225; 29869; 50776; 60441; 224023; 13682;
51789; 207806; 68895; 71735; 109305; 74198; 546078; 216560; 224530; 20924; 15204; 233726; 19881; 14960; 14961; 67809; 74519; 74155; 328162; 22404; 319277; 17246; 69773; 102657;
18037; 214854; 56200; 22121; 320309; 70510; 19090; 67199; 81489; 13088; 269951; 330627; 77125; 19672; 67877; 280635; 18534; 17932; 66451; 55944; 319675; 106759; 219024; 23997; 13608;
71664; 208718; 474145; 14859; 11722; 17837; 239796; 14131; 546083; 21807; 67607; 277923; 52717; 66377; 54357; 432502; 277923; 66131; 59026; 72244
mouse subset 2 (b)
11464; 11539; 11540; 11541; 11765; 11699; 11812; 11815; 11816; 11820; 16613; 11840; 11845; 14570; 11863; 11865; 216869; 11973; 12010; 12043; 12047; 12048;
12050; 12062; 12111; 104184; 12189; 50909; 317677; 12313; 12317; 12363; 12370; 12371; 12389; 12400; 12455; 12480; 12527; 20778; 12567; 12568; 12571; 107951;
12575; 12576; 12581; 12591; 12609; 12611; 12631; 12675; 11777; 67300; 17228; 12803; 12843; 12826; 12827; 12828; 12830; 94216; 12836; 23849; 12912; 11909; 12914;
12954; 12995; 13001; 12387; 66473; 109660; 13030; 13035; 13058; 13076; 13074; 13131; 13176; 13179; 13191; 13197; 13198; 13207; 13347; 109754; 13405; 13419;
13488; 13498; 13649; 13653; 75705; 208643; 109901; 50701; 13712; 13717; 13726; 328572; 269587; 13866; 13872; 13982; 14061; 14067; 14069; 58992; 14114; 14118;
14132; 14156; 14158; 110135; 14163; 14178; 14182; 14183; 14228; 56458; 56484; 14248; 192176; 14268; 14281; 56717; 14375; 14388; 14400; 231580; 433273; 14473;
231103; 56316; 14571; 14756; 14773; 14784; 14786; 14783; 14815; 225642; 14827; 227753; 68705; 15078; 15108; 231086; 15126; 15162; 15163; 15194; 433759; 15211;
211323; 15251; 215114; 14963; 15006; 14998; 14999; 14968; 217082; 97165; 15361; 15369; 15379; 15387; 15388; 51810; 15402; 15439; 15499; 193740; 14828; 15481;
15519; 15510; 15528; 81489; 15925; 52668; 15975; 16001; 16005; 16006; 16008; 16009; 16010; 16011; 16012; 29817; 209268; 16150; 16176; 16180; 16184; 16186;
16195; 227288; 12765; 16199; 16153; 16163; 16164; 16201; 16331; 231070; 54131; 27056; 54123; 16367; 16398; 16399; 16400; 16402; 16408; 16409; 16414; 16438;
16451; 16452; 16453; 16476; 16477; 16478; 12521; 16490; 16623; 16621; 16642; 16641; 16643; 16644; 16593; 16647; 16678; 16765; 16816; 16835; 16846; 16847; 16854;
19039; 233187; 16885; 15450; 16906; 16971; 545422; 268977; 17126; 17127; 17128; 17136; 17756; 17181; 17194; 17210; 17246; 17248; 26406; 26407; 12265; 545943;
17339; 17342; 110616; 54601; 110784; 17390; 17395; 17387; 12705; 17698; 15235; 17702; 17777; 17829; 70603; 17863; 17865; 17869; 17874; 17873; 17877; 17901;
107589; 17927; 17936; 17937; 17938; 76936; 17969; 17970; 17975; 17984; 227197; 18002; 18003; 18007; 18019; 18023; 18024; 18032; 18033; 18034; 18035; 18036;
18037; 18045; 18046; 18049; 18073; 18102; 18125; 18127; 18133; 434373; 18175; 78405; 18214; 18393; 18406; 18424; 18439; 18453; 18813; 18479; 18507; 18538;
18582; 18604; 18607; 67199; 18640; 18643; 18645; 18667; 74769; 18712; 18708; 23988; 18740; 18763; 67451; 18791; 18792; 18793; 18805; 18806; 18815; 56188; 18830;
18854; 18949; 18968; 18970; 18971; 18972; 18973; 80905; 20020; 66420; 17749; 18979; 18984; 18736; 19016; 19014; 19035; 67738; 19045; 51792; 73699; 71978;
110854; 19055; 19060; 116847; 18646; 19073; 19074; 18747; 18750; 18753; 18754; 18760; 19089; 18762; 19090; 19091; 26413; 26417; 23939; 26419; 19094; 26420;
26414; 26395; 19106; 19107; 19122; 67763; 22074; 67373; 17174; 19164; 19165; 19166; 19167; 19179; 23996; 19184; 21762; 66998; 53380; 14083; 20459; 19231; 19242;
15170; 19247; 19249; 19264; 58235; 19298; 19299; 69908; 271457; 19353; 19354; 19367; 19358; 19359; 26448; 110157; 109905; 110351; 76108; 19645; 214899; 19650;
19651; 19660; 19662; 19674; 19696; 19697; 19698; 19687; 69263; 106344; 72151; 19736; 212541; 19762; 19769; 68275; 19891; 19983; 76808; 545162; 20091; 20111;
20130; 20181; 20182; 20190; 20193; 20661; 20198; 20194; 545244; 20196; 29857; 20249; 20278; 15529; 53378; 20339; 20344; 56086; 71514; 110809; 20462; 20390;
52551; 20416; 20481; 29856; 67155; 20586; 20587; 20588; 83797; 104069; 20617; 14086; 20662; 20683; 20692; 20375; 20779; 20787; 20788; 20807; 66661; 20815;
20822; 20832; 20846; 20848; 20849; 20850; 20851; 20852; 268980; 20907; 20924; 20963; 270627; 228980; 21345; 226182; 21343; 545600; 26409; 21354; 21355; 21356;
21374; 21390; 21413; 21405; 21423; 21406; 21411; 21453; 21752; 22041; 21419; 13865; 21803; 21804; 21808; 21810; 21812; 21813; 21815; 21817; 21824; 21825; 21833;
21844; 21859; 21869; 21872; 21877; 21885; 21894; 21917; 21926; 21937; 21938; 21954; 21969; 21973; 22059; 27223; 209456; 22062; 21991; 22004; 22027; 22029;
22030; 22031; 22034; 12462; 22065; 22088; 22095; 22117; 66827; 22139; 22145; 434428; 22151; 22166; 54721; 22209; 22196; 22215; 22218; 21429; 12818; 22256;
22283; 22330; 269523; 22337; 22346; 22350; 22359; 22370; 22371; 215280; 22427; 22431; 103573; 22594; 108138; 22596; 22632; 54401; 22627; 22628; 22629; 22631;
235320; 22661; 22642; 16975; 65111; 12227; 22142; 16178; 252870; 214579; 53761; 17135; 227720; 65969; 11489; 27402; 14283; 11737; 20539; 13716; 22685; 17979;
268903; 22284; 100683; 233489; 12545; 319161; 18720; 108083; 23957; 18201; 26554; 109115; 93762; 26430; 16151; 14457; 17165; 20893; 56469; 229776; 228355;
12361; 23992; 21943; 56041; 22061; 17977; 18222; 12703; 56455; 384783; 53356; 78655; 18611; 71609; 11492; 19766; 53869; 192656; 13481; 216233; 12633; 22402;
15183; 18519; 54126; 51886; 234664; 217715; 224045; 67204; 12427; 266692; 12234; 73178; 53886; 21341; 21340; 21339; 53859; 12702; 22385; 70834; 11632; 20540;
17344; 83945; 64685; 116870; 26909; 16800; 56200; 21945; 23942; 14924; 224997; 19326; 30939; 17681; 544804; 17164; 16600; 56404; 69125; 65962; 26941; 18191;
18190; 79456; 108098; 18208; 70208; 234959; 19317; 13666; 18693; 236899; 19099; 71819; 11504; 67384; 29810; 213539; 57267; 12927; 170791; 12304; 20185; 20602;
22032; 56489; 240087; 20970; 100763; 71474; 215445; 20874; 67952; 50868; 50791; 28185; 23881; 103135; 12355; 12460; 56438; 23857; 246133; 69792; 23958; 21353;
18571; 12049; 12125; 27221; 11768; 56459; 22259; 59026; 11867; 74117; 14155; 27061; 12607; 21849; 21681; 106021; 68278; 74326; 268566; 51796; 56399; 56424;
225372; 22143; 227613; 14694; 229615; 233744; 27374; 52858; 57316; 66513; 23897; 108100; 101206; 22193; 12331; 56403; 59035; 17978; 66884; 18432; 23991; 67040;
81601; 12464; 12461; 20411; 217737; 67268; 13992; 20218; 14007; 12469; 67967; 17175; 15505; 23984; 20174; 53328; 19017; 23882; 56070; 19765; 11811; 66212;
67145; 20867; 22630; 231659; 26754; 72432; 223922; 231821; 56532; 71828; 18715; 107568; 66894; 12539; 18769; 170823; 56289; 56460; 75841; 67037; 22770; 235610;
66922; 17532; 19414; 231876; 195046; 243819; 65945; 17064; 56541; 12465; 56381; 170822; 67074; 233789; 56406; 76779; 74105; 20947; 105689; 111173; 68652;
17776; 66865; 74204; 105522; 71972; 103583; 83814; 432424; 382056; 59287; 14299; 50934; 12419; 52615; 104248; 67815; 17151; 23821; 66245; 17872; 76932; 18969;
26570; 58521; 74309; 17133; 73167; 68379; 68098; 58202; 29876; 20466; 103967; 81702; 67811; 106039; 208263; 18457; 56642; 30877; 67608; 20409; 18458; 237038;
16797; 105837; 99889; 226541; 67207; 77622; 73830; 72459; 56626; 64704; 54125; 216963; 56480; 18983; 19183; 53421; 239393; 69865; 54609; 50777; 58859; 58194;
56461; 99480; 382985; 276919; 71704; 50916; 13196; 26894; 16456; 69367; 67941; 56369; 67184; 435766; 26922; 28019; 67220; 66610; 66940; 223691; 29866; 223828;
50794; 53312; 24069; 18099; 67838; 60321; 80707; 80898; 235442; 53857; 94217; 20418; 224796; 57780; 13063; 231637; 24127; 106298; 58800; 20623; 71984; 53325;
72565; 74195; 57743; 208836; 58523; 73247; 52036; 50754; 24071; 98238; 70024; 106766; 52830; 107975; 57342; 56444; 93742; 53896; 66165; 18146; 65254; 27998;
216749; 223696; 11610; 74268; 53868; 240880; 228714; 12211; 77480; 27428; 330286; 228775; 435336; 63873; 23850; 217026; 214901; 16553; 64113; 13418; 18744;
18193; 26374; 53417; 240047; 74498; 69726; 171580; 65079; 72107; 67819; 116972; 445007; 269424; 70675; 73251; 101513; 71740; 78829; 252966; 71149; 16396; 103724; 94043; 20017; 66262;
66118; 77018; 67951; 68177; 217124; 52700; 67014; 235402; 209488; 217169; 16475; 74330; 52428; 66860; 102060; 243362; 63955; 66525; 72692; 60599; 116940; 99167; 140499; 72544; 326619;
224014; 11569; 81703; 71766; 171211; 74020; 140709; 223513; 22360; 218543; 60510; 14836; 209590; 17846; 76654; 27059; 71753; 22437; 68910; 80904; 15257; 234577; 218194; 233877; 218215;
16772; 242093; 73710; 225471; 232946; 227580
mouse subset 3 (b)
232345; 17960; 20716; 67758; 11303; 11350; 76507; 11352; 113868; 11363; 110446; 11419; 11423; 11430; 11431; 11459; 11475; 11461; 11464; 71885; 60595; 56456;
11472; 11486; 11487; 11518; 11520; 11522; 11529; 11535; 11539; 11540; 11541; 11542; 11545; 11549; 11555; 11556; 110355; 320129; 11771; 11772; 11764; 71770;
11765; 14797; 11576; 11595; 11596; 16449; 11606; 11608; 11609; 11622; 11625; 11651; 11652; 17025; 11655; 11657; 11666; 11669; 26874; 11674; 230163; 11676;
11682; 11684; 11689; 11686; 11687; 11648; 11647; 11650; 76768; 11699; 23802; 218038; 30948; 11723; 11723; 11733; 16790; 11739; 12306; 11747; 11749; 11750;
11783; 103161; 319924; 11784; 11785; 11787; 11789; 20219; 11792; 11797; 11796; 11798; 11799; 11803; 11806; 11807; 11808; 238055; 11810; 11812; 11813; 11815;
11816; 11820; 16613; 14102; 14103; 11826; 11828; 11835; 81003; 11839; 11840; 11842; 11843; 66182; 11844; 11845; 11846; 11847; 11848; 11852; 11853; 56212;
192662; 11857; 14570; 11863; 11865; 109689; 216869; 11883; 11886; 17173; 11889; 11890; 27053; 11905; 11906; 11908; 11910; 11911; 11920; 68703; 11928; 232975;
192113; 11932; 11937; 110935; 11973; 11975; 11998; 54140; 26361; 12000; 12010; 12013; 12015; 12017; 12018; 12021; 12028; 12038; 12443; 12043; 12047; 12048;
12050; 12051; 12053; 110279; 12062; 12075; 12097; 12111; 12122; 12124; 12142; 12143; 12144; 104184; 109778; 12153; 12156; 12162; 12169; 67065; 12177; 51800;
329547; 12189; 109880; 12190; 12192; 12193; 12215; 12223; 12224; 12226; 53414; 12261; 12259; 50909; 317677; 12263; 12266; 15139; 383055; 110382; 12279; 12346;
12349; 12353; 12354; 230099; 76459; 12286; 12288; 12305; 12293; 12295; 12296; 12297; 12311; 12313; 12314; 12317; 12322; 108058; 12328; 12796; 12330; 12333;
12334; 12380; 12362; 12366; 12367; 12363; 12368; 12369; 12370; 12371; 12372; 12373; 12359; 12389; 12393; 12395; 12402; 12406; 109857; 12426; 12428; 268697;
51813; 12444; 12445; 12447; 12450; 12452; 12455; 12466; 12480; 12481; 12504; 12507; 12516; 12525; 12527; 12475; 21940; 12487; 12519; 21941; 12491; 20778; 21939;
21947; 12505; 16423; 12506; 333883; 12512; 12514; 12515; 16149; 12518; 72269; 12534; 23834; 107995; 12532; 216150; 12540; 12550; 12558; 12560; 12562; 12563;
12567; 12568; 12571; 107951; 12575; 12576; 12577; 12580; 12581; 74596; 12591; 12606; 12608; 12609; 110794; 12611; 104158; 12631; 12632; 13032; 12638; 83702;
12649; 12652; 243764; 12671; 11438; 12675; 11769; 11777; 23844; 12723; 12724; 12725; 12728; 26372; 26373; 12759; 114584; 12748; 12729; 12757; 67300; 17228;
12768; 12772; 12771; 12773; 12774; 16995; 12780; 333329; 12795; 12797; 12803; 12805; 12842; 12843; 12824; 12825; 12826; 12830; 94216; 12831; 12832; 12833;
12834; 12836; 12839; 12840; 12841; 12814; 12821; 12846; 70349; 23849; 12870; 76703; 56373; 12902; 12903; 12904; 12912; 11909; 12914; 12921; 68337; 12928; 12944;
12954; 12970; 26416; 12977; 12978; 12981; 12982; 12984; 12985; 12986; 12988; 93687; 104318; 27373; 12995; 13000; 13001; 13003; 121021; 13008; 14219; 107869;
12385; 12387; 12388; 18163; 66473; 109660; 13030; 13033; 13034; 13035; 13036; 13038; 13040; 13047; 109672; 13057; 13058; 66445; 13076; 13077; 13078; 13113;
13088; 13098; 72303; 13095; 72082; 13106; 13113; 337924; 13120; 13122; 13070; 13072; 13074; 13075; 13079; 104086; 13121; 13131; 13132; 13136; 223453; 69635;
13163; 13166; 13176; 13179; 11421; 13191; 13194; 107986; 77337; 77337; 13197; 13198; 13207; 13211; 11790; 13347; 74754; 13361; 109754; 18104; 235339; 13382;
13383; 13385; 13393; 13394; 13405; 13406; 13429; 13419; 13430; 13433; 21673; 13482; 13488; 13489; 13490; 13496; 13498; 13507; 14605; 109620; 13517; 15200;
19252; 13537; 319520; 240672; 235584; 110074; 13542; 13555; 104394; 13559; 13560; 230857; 13605; 13607; 14745; 13614; 13618; 13627; 67160; 13629; 13645; 13649;
13653; 13654; 13655; 13656; 66235; 13665; 209354; 26905; 13836; 13681; 75705; 13684; 13685; 13688; 208643; 276770; 109901; 50701; 15569; 15568; 56501; 13711;
13712; 13714; 13717; 13726; 13043; 13807; 63959; 328572; 13819; 269587; 13844; 13845; 13849; 13856; 13857; 107508; 13860; 13866; 13867; 13869; 13871; 13872;
22592; 319955; 13876; 78943; 14113; 13982; 13983; 26380; 23871; 23872; 104156; 14011; 14027; 14030; 14055; 14056; 14061; 14062; 14063; 14066; 14067; 14069;
14058; 109821; 58992; 14073; 14079; 14081; 74205; 50790; 19229; 14089; 14114; 14104; 14118; 14128; 14129; 14131; 14130; 246256; 14132; 110196; 14156; 14158;
14159; 110135; 14163; 14173; 14178; 14182; 14184; 14183; 14186; 99571; 14198; 14200; 14225; 14226; 14227; 14228; 14229; 17300; 15229; 110805; 14235; 56458;
56484; 433621; 14247; 14248; 77836; 192176; 14254; 14255; 55990; 14262; 226564; 14263; 14268; 14281; 14282; 14284; 14293; 14297; 56717; 14309; 26424; 14319;
26423; 14459; 233908; 14360; 14381; 14385; 14375; 14388; 14390; 14399; 14400; 14408; 231580; 14432; 433273; 14453; 14460; 14461; 14465; 14466; 14473; 14526;
14528; 320415; 231103; 14534; 17700; 14580; 11692; 56316; 14598; 207182; 14600; 14609; 14612; 14620; 12091; 14632; 14667; 66355; 14673; 14676; 14681; 14683;
14688; 14695; 99412; 14718; 14719; 55948; 14723; 14724; 14729; 54368; 14733; 14571; 14751; 14756; 12766; 14772; 14773; 17347; 216860; 76282; 14775; 14779;
14784; 14786; 14799; 14810; 14811; 14815; 20310; 330122; 225642; 14827; 56637; 227753; 14782; 14863; 14864; 14866; 14871; 17688; 83602; 229906; 74197; 68153;
98053; 68705; 14884; 23894; 14885; 14886; 72308; 54195; 14938; 14939; 14958; 107970; 15270; 51788; 15078; 15108; 231086; 15122; 15122; 15130; 15129; 15135;
15132; 544763; 15126; 15162; 15163; 15194; 433759; 15182; 15211; 15212; 12628; 15216; 50702; 15234; 211323; 15242; 15248; 15251; 215114; 53323; 15277; 14963;
110557; 15006; 14998; 14999; 15001; 15002; 14960; 14968; 14969; 14969; 217082; 15040; 15013; 15007; 15289; 97165; 15357; 15361; 15368; 15369; 15370; 15375;
15376; 15377; 15378; 15379; 225307; 53379; 15384; 15381; 59013; 15387; 15388; 51810; 15401; 15402; 15395; 15415; 15417; 15423; 15426; 15439; 15452; 15461;
15468; 15469; 15205; 15492; 15483; 15484; 15485; 15488; 15499; 15502; 193740; 193740; 15512; 15525; 14828; 15481; 15526; 15507; 69253; 15519; 15516; 15510;
15528; 81489; 15574; 15874; 15891; 15894; 15896; 15901; 15902; 15903; 15925; 319554; 15951; 52668; 545342; 15974; 15975; 15976; 15977; 15978; 15979; 15980;
18518; 16000; 16001; 16002; 16004; 16005; 16006; 16008; 16009; 16010; 16011; 16012; 29817; 16007; 19664; 209268; 16150; 16175; 16176; 16180; 16181; 16183;
16184; 16185; 16186; 16189; 16190; 16191; 16193; 16194; 16195; 16197; 227288; 12765; 16199; 16153; 16154; 16161; 16163; 16164; 16168; 16170; 16173; 67781;
16201; 16202; 26356; 16322; 15945; 16334; 16331; 16332; 231070; 16341; 18609; 16351; 16179; 16403; 546132; 16362; 16363; 54131; 16364; 27056; 54139; 54123;
16367; 16392; 16398; 16399; 16400; 16401; 16402; 16407; 16408; 16409; 16410; 16412; 16414; 16416; 192897; 16418; 16419; 16421; 16434; 16438; 16447; 16451;
16452; 16453; 16476; 16477; 16478; 16480; 12521; 85305; 16485; 16490; 16491; 16492; 16494; 16500; 16508; 56543; 16509; 16510; 16523; 16514; 16531; 140493;
16534; 16535; 16542; 16590; 16623; 16621; 16642; 16641; 16643; 16644; 16593; 16211; 16647; 16653; 16678; 110308; 110310; 16691; 268482; 16666; 16668; 16669;
16728; 16773; 16777; 226519; 16785; 16765; 98386; 16816; 16818; 16819; 16828; 16835; 16846; 16847; 16852; 107753; 16854; 19039; 16855; 56048; 16859; 16878;
16881; 319583; 233187; 16885; 16886; 15450; 16890; 70361; 16905; 16906; 16909; 16956; 16971; 545422; 16976; 16992; 16994; 72054; 268977; 17000; 17002; 17022;
17096; 20400; 17105; 56753; 17075; 17113; 17118; 17119; 56150; 17125; 17126; 17127; 17128; 17130; 17131; 17136; 73744; 17159; 17161; 109731; 17756; 17758;
232087; 17187; 17189; 17190; 17194; 84004; 109904; 17210; 17221; 17242; 17246; 17248; 17258; 17261; 54483; 26401; 26406; 26407; 26408; 17283; 17287; 17295;
17150; 17314; 17313; 12265; 17318; 545943; 17329; 17339; 17342; 110616; 17345; 17350; 269881; 26403; 214162; 64144; 110784; 17380; 83995; 17390; 17392; 17393;
17394; 17395; 17384; 17385; 17386; 17387; 58223; 433938; 17420; 17523; 17533; 17535; 17250; 12705; 17685; 17686; 17698; 15235; 17701; 17702; 17750; 66902;
17708; 17709; 17766; 17769; 17721; 17777; 17829; 546017; 70603; 17855; 17859; 17863; 17865; 17869; 18109; 17874; 17873; 140781; 17880; 17901; 17906; 17897;
107589; 432516; 17913; 17918; 17926; 17927; 17928; 17936; 17937; 17938; 76936; 17940; 17965; 27354; 17969; 17970; 17972; 17973; 17975; 17984; 17991; 67130;
66416; 66495; 66046; 67264; 227197; 18002; 18003; 18007; 18012; 18015; 18016; 18019; 18027; 18022; 18023; 18024; 18028; 18029; 18032; 18033; 18034; 18035;
18036; 18037; 18044; 18045; 18046; 18049; 434401; 18073; 18102; 18103; 18105; 110109; 53610; 18125; 18126; 18127; 18128; 18129; 18133; 434373; 230899; 18159;
230103; 18162; 18175; 230598; 18181; 22608; 23959; 78405; 18207; 18211; 18212; 18213; 18214; 18216; 101706; 269966; 246727; 209815; 18245; 18247; 18294; 74143;
18383; 18390; 18392; 18393; 18406; 18413; 18416; 18424; 18429; 18430; 228139; 18439; 18442; 18453; 18813; 18550; 18477; 18787; 18788; 18479; 224105; 18481;
18489; 50873; 74108; 114774; 18504; 18505; 18507; 13180; 23983; 18521; 268591; 18534; 18536; 18537; 18538; 18548; 18549; 18573; 54611; 238871; 18590; 18595;
18596; 18597; 18598; 68263; 228026; 18604; 236900; 27273; 18605; 18607; 18613; 20317; 18616; 71382; 103737; 67199; 18637; 109054; 56612; 18640; 170768; 18643;
18645; 18667; 18671; 18670; 18673; 20704; 20724; 20725; 20723; 18706; 74769; 18712; 30955; 18708; 224020; 23988; 18740; 18763; 18764; 18746; 18783; 18791;
18792; 18793; 18797; 18799; 18803; 18805; 18806; 18810; 18815; 18816; 18817; 56188; 18824; 433328; 18830; 18854; 227099; 18861; 18949; 18968; 18970; 18971;
18972; 18973; 80905; 20020; 66420; 17749; 18976; 18979; 330260; 269823; 18984; 18736; 18986; 19016; 19014; 433914; 19035; 67738; 19041; 19042; 19045; 19047;
100336; 19052; 19053; 51792; 73699; 71978; 72930; 269643; 235542; 110854; 226849; 19055; 56420; 19056; 19060; 67857; 19065; 18646; 19073; 19074; 19075; 105787;
108079; 18747; 18749; 18767; 18768; 19087; 18750; 18751; 18753; 18754; 18755; 18759; 320795; 109333; 18760; 18761; 19089; 18762; 19090; 19091; 26413; 26417;
50772; 23939; 26419; 26420; 26414; 26395; 26396; 23938; 26400; 19106; 19107; 72981; 19109; 19116; 19122; 19123; 19130; 19132; 110639; 67763; 114228; 22074;
67373; 17174; 19699; 19146; 19144; 56213; 19152; 19164; 19165; 244631; 26440; 19166; 19167; 26441; 26442; 26443; 26444; 19170; 26445; 26446; 19172; 19173;
19175; 19177; 16913; 16912; 19171; 19179; 19181; 19182; 23996; 19184; 67089; 21762; 19185; 66998; 17463; 67151; 53380; 19186; 19188; 19204; 19205; 19206; 19211;
19217; 19219; 19226; 19224; 19225; 19228; 14083; 20459; 19230; 19231; 19242; 19246; 19255; 15170; 320139; 19247; 19249; 19262; 19264; 19267; 19268; 19271;
19274; 19277; 19283; 19355; 58235; 19298; 19299; 19300; 19305; 69908; 19341; 271457; 26412; 19352; 19353; 19354; 19367; 19356; 19358; 19359; 19361; 26448;
110157; 19373; 56044; 19386; 109905; 110351; 76108; 19401; 218772; 19411; 218397; 19417; 19645; 19646; 245688; 19650; 19651; 19659; 19662; 19674; 19133; 19684;
19696; 19697; 19698; 19702; 19703; 19704; 19712; 19713; 19687; 69263; 106344; 72151; 19735; 19736; 212541; 93726; 24014; 24015; 19877; 19883; 225998; 68275;
19891; 27367; 19983; 76808; 19941; 545162; 57808; 56040; 67186; 27050; 20091; 20104; 20111; 20112; 110651; 58988; 20115; 545151; 20042; 20068; 78294; 20130;
20133; 20135; 20181; 20182; 20187; 20190; 20191; 20193; 20661; 20198; 545244; 20196; 20203; 20208; 20211; 29857; 67125; 20229; 20239; 20249; 20271; 20276;
20278; 20293; 20303; 20304; 20306; 20292; 20295; 20297; 20298; 20299; 56066; 16963; 20312; 20969; 15529; 53378; 20315; 67680; 66226; 20339; 20343; 20344; 20345;
26398; 56086; 71514; 110809; 20382; 67996; 225027; 20462; 20387; 20390; 20393; 52551; 20415; 20416; 20481; 20536; 20525; 20526; 56485; 20532; 17254; 20533;
29856; 114479; 13162; 21366; 20544; 105243; 20496; 20509; 20515; 20568; 67155; 20586; 20587; 20588; 83797; 20595; 20595; 20610; 434241; 104069; 20617; 20618;
14086; 53607; 20638; 545845; 20656; 20657; 20662; 20663; 20682; 20683; 20687; 20692; 20375; 20730; 20737; 20750; 20766; 20739; 20779; 78925; 94224; 20787;
20788; 20807; 66661; 20822; 20823; 20832; 20604; 20607; 20846; 20847; 20848; 20849; 20850; 20851; 20852; 20853; 20860; 56274; 58231; 20878; 20869; 268980;
20907; 56389; 20910; 20887; 54200; 20922; 20924; 225115; 20963; 20964; 11491; 270627; 228980; 21345; 226182; 21343; 545600; 26409; 21354; 21355; 21356; 21374;
21390; 21391; 67923; 67673; 21405; 21410; 21423; 21417; 21411; 21427; 21454; 21672; 21685; 21745; 21752; 21754; 22041; 21780; 21418; 21419; 21420; 13865; 11819;
21781; 21786; 21802; 21803; 21804; 21808; 21809; 320202; 21810; 21812; 21813; 21815; 21817; 21824; 21825; 50492; 21832; 21833; 21834; 21838; 21841; 21843;
21844; 21857; 21858; 21859; 110595; 21869; 21872; 21877; 21885; 21894; 24088; 142980; 21898; 21916; 21917; 21926; 21929; 21937; 21938; 21952; 21954; 81877;
21969; 21973; 21974; 22059; 27223; 209456; 22062; 21991; 22004; 22018; 22027; 22029; 22030; 22031; 22033; 22034; 12462; 56191; 22065; 22068; 64930; 22084;
22088; 22095; 22117; 66827; 56354; 50500; 22139; 22145; 434428; 22151; 22160; 22164; 22166; 50493; 54721; 22171; 547099; 22190; 74153; 22209; 216080; 56550;
66105; 22194; 67128; 22196; 22195; 22215; 22218; 21429; 22227; 22228; 22229; 22232; 100727; 231396; 243085; 71773; 22239; 12818; 22256; 22260; 22271; 22278;
22282; 22283; 22327; 22329; 22330; 269523; 22333; 22337; 22339; 22346; 22350; 22352; 22359; 24115; 22367; 26950; 22370; 22371; 22376; 215280; 22390; 24116;
22408; 22413; 22427; 22431; 22436; 22590; 22591; 103573; 22594; 74335; 108138; 22596; 22632; 54401; 22627; 22628; 22629; 22631; 22668; 22695; 20841; 235320;
22661; 22642; 228913; 22785; 16975; 229663; 12227; 57377; 22142; 16178; 12767; 252870; 19349; 214579; 53761; 224727; 434585; 53817; 193742; 27226; 17135;
21789; 64213; 18124; 227720; 16871; 65969; 11489; 13009; 27402; 14283; 75717; 19244; 22225; 64050; 15364; 11737; 20539; 11640; 13716; 22685; 68188; 17979;
268903; 22284; 56292; 100683; 233489; 80911; 12005; 72399; 12545; 17120; 18720; 108083; 23957; 223920; 18201; 69181; 99375; 26554; 71745; 194655; 109115;
93762; 94244; 108155; 26430; 16498; 16151; 14457; 170716; 52163; 14160; 20893; 56469; 229776; 21393; 228355; 17346; 12361; 110198; 23992; 21943; 56505; 19012;
56041; 22061; 108112; 105349; 17977; 18222; 12703; 56455; 212647; 384783; 13669; 53356; 54709; 78655; 53330; 74732; 18611; 108014; 13350; 71609; 85030; 56453;
11492; 19766; 12123; 50930; 69583; 22035; 11490; 11502; 110911; 230979; 53869; 192656; 14082; 21933; 18632; 12454; 23825; 29875; 216233; 12633; 22402; 15183;
18519; 18171; 104015; 15937; 54126; 51886; 234664; 13667; 108067; 217715; 224045; 67204; 12427; 108012; 11766; 11767; 83767; 12570; 11776; 12234; 73178; 53886;
15114; 21341; 21340; 21339; 53859; 12702; 69454; 22385; 11632; 232431; 233406; 20540; 13664; 268930; 83945; 64685; 116870; 12448; 54189; 27966; 67526; 15239;
104245; 26909; 18554; 53978; 17082; 18414; 56200; 21945; 30960; 23942; 224997; 19326; 30939; 17681; 56791; 73086; 544804; 17164; 19159; 19158; 19157; 14739;
69207; 16600; 69125; 20624; 65019; 65962; 16870; 26941; 11450; 18189; 79456; 17444; 21873; 13123; 18208; 26357; 70208; 19317; 16432; 13666; 24056; 236899;
26987; 11793; 16541; 19099; 71819; 94275; 240913; 11504; 73078; 23886; 67384; 64292; 57267; 100198; 12927; 217866; 170791; 11758; 227929; 12304; 18025; 20185;
20602; 56640; 56489; 56468; 240087; 433631; 20970; 100763; 215445; 53890; 232370; 20874; 17184; 67952; 50868; 17279; 329504; 28185; 23881; 29809; 78455; 54338;
14170; 12355; 20186; 12460; 353187; 56438; 14181; 23857; 107723; 246133; 23797; 23958; 170789; 12049; 12125; 78733; 27221; 14615; 11768; 56459; 74006; 22259;
16068; 59026; 11867; 74117; 66713; 27219; 19360; 14155; 320982; 68015; 27061; 67057; 22057; 50498; 12607; 21849; 242687; 21681; 19192; 54598; 12695; 106021;
68278; 69274; 211770; 66905; 74326; 72413; 268566; 51796; 24063; 24064; 51801; 230709; 56399; 56424; 20842; 56445; 70584; 225372; 14767; 320832; 17540; 22143;
22153; 227613; 50768; 17988; 14694; 229615; 22601; 233744; 27374; 230661; 57316; 53333; 23897; 108100; 71890; 101206; 22193; 53319; 12331; 56403; 59035; 17978;
66884; 18432; 23991; 81601; 233726; 67134; 19124; 24060; 20531; 12464; 12461; 20411; 217737; 56376; 140486; 68603; 13992; 20218; 13046; 14007; 13018; 14674;
12469; 28035; 67967; 20499; 20873; 26405; 17175; 18008; 57259; 20620; 15505; 23984; 20301; 15442; 20174; 53328; 13116; 19017; 240354; 23882; 110147; 19765;
11811; 11757; 94215; 12412; 66212; 67145; 216440; 20867; 216197; 22630; 26754; 56307; 72432; 73690; 223922; 56431; 56532; 71828; 18715; 66894; 244486; 23794;
16568; 104445; 12539; 18769; 13404; 170823; 19245; 101739; 217578; 56289; 50883; 26447; 63953; 94062; 66489; 56078; 67037; 80915; 77113; 106648; 101488; 57320;
106504; 56486; 18074; 66922; 24128; 17532; 107503; 19414; 231876; 195046; 65945; 17064; 226641; 13380; 12465; 69807; 55982; 232533; 320632; 170822; 233789;
56406; 76779; 74105; 67088; 94253; 105689; 226169; 111173; 260302; 66615; 14376; 66865; 217893; 83410; 103583; 216739; 83814; 432424; 21981; 382056; 59287;
93759; 14299; 74053; 50934; 104215; 15213; 12419; 20335; 52615; 104248; 75956; 67815; 17151; 12488; 23821; 26434; 66245; 17087; 17872; 76932; 18969; 26570;
56363; 170755; 58521; 74309; 17133; 73167; 68379; 30051; 56175; 30945; 68098; 58202; 29876; 20466; 20399; 244152; 70231; 106039; 208263; 56407; 66870; 19260;
18457; 80857; 56642; 80888; 67608; 18130; 56321; 20409; 18458; 76884; 12070; 237038; 16797; 105837; 102162; 15467; 170770; 72318; 99889; 209416; 18569; 226541;
77622; 72459; 56626; 64704; 54125; 108115; 108116; 28253; 217410; 13169; 66824; 56480; 26934; 223870; 18983; 19183; 13631; 13854; 53421; 212898; 18599; 83768;
69865; 54609; 212999; 58859; 58194; 56461; 70357; 69165; 99480; 192157; 382985; 14373; 50490; 224480; 140494; 276919; 71704; 50916; 26894; 16456; 69367; 57441;
56369; 67184; 226548; 67877; 57875; 26891; 72999; 235661; 28019; 16842; 67220; 50497; 18080; 243816; 66610; 66940; 170743; 68185; 223691; 22368; 66616; 66881;
29866; 29870; 223828; 83430; 59004; 83701; 50794; 53312; 18099; 433256; 67838; 60321; 103149; 58991; 80707; 80898; 70415; 53857; 94217; 224796; 99439; 51786;
224440; 81897; 13063; 74155; 15112; 231637; 108086; 381511; 56057; 58800; 20623; 71984; 53325; 76375; 57743; 50754; 67073; 24071; 64177; 381319; 269870; 67072;
107975; 235281; 66340; 56444; 67388; 53896; 194352; 66593; 66165; 57264; 56632; 54645; 94232; 77531; 216749; 223696; 11610; 74268; 27281; 53868; 68891; 240880;
215303; 228714; 29812; 74770; 57138; 11624; 239985; 77480; 116871; 74370; 382051; 57775; 74018; 74521; 228775; 56195; 268973; 70008; 435336; 63873; 245527;
23850; 17828; 214901; 16553; 64085; 13824; 77684; 57914; 64113; 257632; 233781; 13418; 18744; 18193; 26374; 53417; 240047; 23885; 227525; 67841; 74498; 26895;
69726; 217364; 171580; 70839; 18100; 234593; 65079; 75607; 94213; 68520; 171382; 72107; 67819; 74616; 78514; 110350; 76793; 102580; 116972; 445007; 269424;
70675; 96979; 74838; 80751; 68634; 229644; 77629; 67395; 13002; 80334; 66427; 12822; 73251; 192775; 14950; 101513; 67180; 68559; 71740; 66899; 78829; 252966;
208117; 71149; 16396; 103724; 69742; 74178; 229317; 94043; 56297; 67235; 69538; 224619; 77832; 66262; 66911; 66118; 66734; 77018; 73470; 68177; 217124; 353025; 108682; 52700; 16907;
67014; 235402; 209488; 217169; 16475; 74330; 52428; 71701; 224814; 170738; 102060; 68876; 63955; 320405; 60599; 116940; 112406; 112407; 207214; 66113; 140499; 68214; 170574; 326619;
224014; 11569; 81703; 171282; 69875; 171211; 241431; 384185; 211329; 140709; 223513; 68097; 22360; 60510; 14836; 106759; 209590; 99311; 76654; 27059; 67379; 71753; 22437; 68910; 118449;
15257; 71138; 233877; 218215; 16772; 544954; 71887; 242093; 225471; 407243; 227580

TABLE 3A
Other toxicity biomarkers tcdd; hl, ez, hg
human subset 1 (tcdd)

| | | |
|---|---|---|
| 440837 | LOC440837 | similar to Apolipoprotein A-I precursor (Apo-AI) |
| 10018 | BCL2L11 | BCL2-like 11 (apoptosis facilitator) |
| 839 | CASP6 | caspase 6, apoptosis-related cysteine peptidase |
| 948 | CD36 | CD36 antigen (collagen type I receptor, thrombospondin receptor) |
| 960 | CD44 | CD44 antigen (homing function and Indian blood group system) |
| 1543 | CYP1A1 | cytochrome P450, family 1, subfamily A, polypeptide 1 |
| 2052 | EPHX1 | epoxide hydrolase 1, microsomal (xenobiotic) |
| 2194 | FASN | fatty acid synthase |
| 2619 | GAS1 | growth arrest-specific 1 |
| 2729 | GCLC | glutamate-cysteine ligase, catalytic subunit |
| 2768 | GNA12 | guanine nucleotide binding protein (G protein) alpha 12 |
| 2805 | GOT1 | glutamic-oxaloacetic transaminase 1, soluble (aspartate aminotransferase 1) |
| 221357 | GSTA5 | glutathione S-transferase A5 |
| 3120 | HLA-DQB2 | major histocompatibility complex, class II, DQ beta 2 |
| 3127 | HLA-DRB5 | major histocompatibility complex, class II, DR beta 5 |
| 972 | CD74 | CD74 antigen (invariant polypeptide of major histocompatibility complex, class II antigen-associated) |
| 2171 | FABP5 | fatty acid binding protein 5 (psoriasis-associated) |
| 4023 | LPL | lipoprotein lipase |
| 4609 | MYC | v-myc myelocytomatosis viral oncogene homolog (avian) |
| 4616 | GADD45B | growth arrest and DNA-damage-inducible, beta |
| 1728 | NQO1 | NAD(P)H dehydrogenase, quinone 1 |
| 4851 | NOTCH1 | Notch homolog 1, translocation-associated (Drosophila) |
| 5105 | PCK1 | phosphoenolpyruvate carboxykinase 1 (soluble) |
| 8737 | RIPK1 | receptor (TNFRSF)-interacting serine-threonine kinase 1 |
| 9249 | DHRS3 | dehydrogenase/reductase (SDR family) member 3 |
| 7127 | TNFAIP2 | tumor necrosis factor, alpha-induced protein 2 |
| 7358 | UGDH | UDP-glucose dehydrogenase |
| 7498 | XDH | xanthine dehydrogenase |
| 10007 | GNPDA1 | glucosamine-6-phosphate deaminase 1 |
| 11001 | SLC27A2 | solute carrier family 27 (fatty acid transporter), member 2 |
| 55502 | HES6 | hairy and enhancer of split 6 (Drosophila) |
| 9663 | LPIN2 | lipin 2 |
| 57406 | ABHD6 | abhydrolase domain containing 6 |
| 2645 | GCK | glucokinase (hexokinase 4, maturity onset diabetes of the young 2) |
| 874 | CBR3 | carbonyl reductase 3 |

human subset 2 (tcdd)
841; 843; 972; 1277; 1278; 1445; 1462; 1758; 1832; 1839; 1956; 2035; 2066; 2247; 2534; 2646; 3064; 3108; 3109; 3303; 3320; 3329; 3486; 3932; 4001; 4035; 4036; 4067;
4145; 4282; 4316; 4318; 4691; 4771; 4801; 4802; 4856; 4869; 4999; 5111; 5208; 5294; 5366; 5371; 5430; 5465; 5467; 5468; 5518; 5536; 5552; 5578; 5595; 5598; 5599;
5604; 5891; 5894; 5922; 5925; 5933; 5970; 6278; 6401; 6598; 6667; 6696; 6714; 6908; 6915; 7006; 7021; 7046; 7048; 7057; 7074; 7161; 7253; 7277; 7278; 7373; 7410; 7430; 7436; 7520; 7525; 7528; 7709;
7846; 8174; 8202; 8295; 8464; 8650; 8738; 8772; 8788; 8795; 8797; 8837; 8850; 8878; 8880; 9013; 9014; 9015; 9020; 9043; 9111; 9138; 9794; 9826; 10376; 10475; 10524; 11035; 11214; 22938; 23077;
23136; 23365; 26292; 50484; 51176; 51807; 54476; 54567; 55294; 57448; 83737; 84864; 84875; 90480; 113878; 125168; 407975
human subset 3 (tcdd)
10; 43; 86; 100; 102; 133; 155; 174; 182; 196; 207; 238; 240; 274; 320; 322; 335; 344; 345; 348; 351; 355; 356; 387; 429; 571; 572; 578; 581; 595; 596; 598; 604; 633; 637;
638; 672; 696; 835; 836; 840; 841; 842; 843; 859; 873; 891; 898; 904; 912; 914; 928; 949; 958; 960; 963; 972; 983; 990; 999; 1000; 1003; 1013; 1019; 1026; 1027; 1029;
1050; 1051; 1071; 1129; 1137; 1272; 1277; 1278; 1282; 1387; 1437; 1445; 1457; 1462; 1499; 1500; 1504; 1528; 1543; 1544; 1545; 1555; 1559; 1571; 1576; 1586; 1647;
1728; 1758; 1816; 1839; 1840; 1869; 1874; 1875; 1915; 1950; 1956; 1958; 1969; 2017; 2035; 2053; 2064; 2066; 2068; 2099; 2100; 2119; 2149; 2168; 2169; 2247; 2272;
2309; 2315; 2321; 2353; 2495; 2534; 2548; 2551; 2623; 2626; 2646; 2648; 2678; 2697; 2730; 2770; 2776; 2806; 2875; 2885; 2896; 2932; 2939; 2944; 2946; 2950; 2952;
2962; 2968; 2969; 3002; 3015; 3020; 3064; 3075; 3082; 3091; 3108; 3109; 3117; 3119; 3122; 3123; 3124; 3125; 3126; 3161; 3162; 3172; 3183; 3184; 3263; 3265; 3267;
3280; 3303; 3320; 3329; 3336; 3384; 3458; 3481; 3482; 3486; 3491; 3492; 3516; 3562; 3576; 3630; 3667; 3673; 3674; 3676; 3678; 3683; 3684; 3685; 3688; 3689; 3690;
3691; 3714; 3725; 3845; 3875; 3932; 3949; 3955; 3990; 3991; 4035; 4036; 4043; 4067; 4084; 4087; 4088; 4089; 4137; 4145; 4149; 4162; 4170; 4193; 4215; 4216; 4233;
4242; 4255; 4257; 4282; 4288; 4292; 4297; 4313; 4314; 4316; 4318; 4323; 4363; 4436; 4478; 4524; 4582; 4601; 4602; 4605; 4610; 4613; 4683; 4691; 4706; 4771; 4780;
4790; 4800; 4801; 4802; 4830; 4831; 4835; 4846; 4853; 4854; 4855; 4856; 4869; 4904; 4907; 4916; 5045; 5054; 5058; 5093; 5094; 5111; 5130; 5204; 5208; 5294; 5320;
5321; 5328; 5335; 5465; 5468; 5493; 5518; 5536; 5552; 5567; 5578; 5585; 5590; 5595; 5598; 5599; 5601; 5604; 5617; 5618; 5663; 5728; 5743; 5747; 5871; 5879; 5891;
5894; 5921; 5922; 5925; 5933; 5934; 5962; 5970; 6199; 6278; 6304; 6319; 6401; 6403; 6431; 6464; 6500; 6514; 6521; 6598; 6648; 6667; 6670; 6693; 6696; 6711; 6714;
6720; 6772; 6817; 6868; 6915; 6927; 7006; 7012; 7015; 7020; 7021; 7027; 7037; 7040; 7046; 7048; 7057; 7074; 7088; 7099; 7124; 7128; 7132; 7133; 7150; 7157; 7161;
7185; 7186; 7187; 7188; 7189; 7277; 7278; 7341; 7373; 7391; 7392; 7410; 7412; 7416; 7422; 7424; 7430; 7431; 7436; 7486; 7515; 7517; 7525; 7528; 7704; 7709; 7763;
7764; 7846; 8061; 8174; 8209; 8295; 8379; 8410; 8431; 8464; 8465; 8473; 8488; 8489; 8517; 8607; 8650; 8655; 8714; 8717; 8738; 8743; 8767; 8772; 8775; 8788; 8795; 8797; 8837; 8850; 8878; 8880; 9020;
9111; 9138; 9253; 9354; 9370; 9603; 9612; 9794; 9826; 9891; 10138; 10260; 10277; 10376; 10475; 10524; 10575; 10642; 10664; 10672; 10855; 10892; 10912; 10998; 11035; 11086; 11124; 11213; 11214;
22938; 23013; 23077; 23136; 23175; 23365; 23671; 26292; 27097; 27113; 27349; 28514; 28965; 51129; 51176; 51807; 54093; 54476; 54567; 55294; 55759; 64900; 83737; 84864; 84875; 87178; 90480; 94025;
121504; 132789; 376497; 386619; 406952; 406982; 407975
chimpanzee subset 1 (tcdd)
451564; 459487; 457777; 454986; 462776; 463234; 462595; 462594; 462191; 472619; 464031; 468012; 469814; 471171; 470347; 462155; 453423; 455317; 470830; 458533
chimpanzee subset 2 (tcdd)
460110; 459873; 462191; 463542; 461766; 462420; 456689; 459919; 459107; 449592; 459852; 463396; 452006; 459713; 472771; 455592; 451509; 452191; 456799;
462261; 459868; 458081; 457687; 463649; 453736; 460178; 468983; 456150; 450503; 454037; 450445; 453526; 453176; 460182; 460741; 452715; 466665; 469501;
450191; 458702; 451939; 461366; 458229; 468667; 461343; 462758; 472992; 460243; 453319; 470654; 464967; 449614; 455322; 451875; 458308; 463564; 453014;
452128; 451389; 467556; 459872; 460219; 456968; 454279; 459022; 454750; 470714; 456067; 471873; 451329; 452820; 453616; 453073; 452608; 455309; 451618;
469288; 472832; 461425; 453344; 459138; 458191; 460541; 472888
chimpanzee subset 3 (tcdd)
453472; 451021; 464113; 461258; 453202; 459126; 459590; 472947; 451564; 466149; 469584; 458500; 451291; 462616; 458156; 473829; 458635; 470232; 449497;
471892; 463804; 450745; 460110; 456457; 459873; 458531; 461835; 455914; 469524; 449512; 451775; 452359; 462191; 450481; 468234; 468509; 468076; 452025;
465021; 455931; 469969; 467982; 463758; 469999; 451836; 463542; 467891; 471627; 461766; 451203; 468580; 453745; 456052; 450626; 450857; 456929; 468012;
473340; 458184; 454158; 472057; 462097; 451391; 456689; 464074; 454636; 459919; 456124; 463079; 460906; 471510; 470835; 464984; 453047; 454940; 465619;
459107; 454677; 470168; 467989; 454728; 470895; 462769; 451717; 455639; 462549; 463472; 461400; 457782; 457602; 463503; 452958; 449592; 471974; 462591;
462594; 470195; 458269; 461216; 450992; 466302; 459990; 460942; 459852; 452179; 449517; 466318; 463112; 463396; 456988; 461152; 471626; 471205; 450954;
454730; 470596; 451958; 459807; 450396; 458607; 468295; 456893; 473387; 455722; 472272; 467694; 452006; 459713; 461086; 472771; 470395; 455407; 468537;
455592; 452972; 457274; 468286; 463119; 463671; 458811; 473380; 450866; 471095; 451587; 454094; 451511; 451509; 459215; 466199; 463016; 458262; 459043;
464278; 459769; 461406; 452191; 456799; 455134; 462395; 455792; 462587; 462261; 470311; 462864; 453652; 472467; 451439; 459300; 467006; 458081; 471049;
451938; 457687; 463649; 456587; 460178; 453893; 468983; 456150; 450503; 469367; 454037; 450445; 453526; 462468; 467503; 466142; 464422; 463253; 453176;
460182; 461762; 460741; 452715; 467978; 466776; 466665; 469501; 460213; 450676; 450191; 469946; 457351; 462063; 460848; 458702; 449634; 451939; 459750;
467939; 461366; 459234; 458229; 470721; 454013; 459018; 468667; 461343; 471443; 462758; 460956; 456060; 472992; 460243; 453319; 472136; 466934; 457968; 472883; 464702; 464876; 453178; 457711;
450474; 470654; 459882; 457443; 468835; 471632; 471358; 464967; 472732; 456094; 453191; 455322; 451557; 464527; 451875; 458598; 463564; 465702; 453014; 455128; 454154; 452128; 460852; 464277;
451389; 456168; 467556; 459872; 460219; 456968; 454750; 470714; 456067; 451586; 471032; 463298; 467150; 466958; 467708; 471873; 451329; 461206; 468549; 464585; 456351; 452820; 471354; 456846;
467058; 453616; 453073; 456468; 452608; 455309; 459036; 451618; 469288; 470233; 465121; 462038; 461425; 458530; 453344; 459888; 458191; 460541; 472888; 459247; 455841

human subset 1 (hl)

| | | |
|---|---|---|
| 30 | ACAA1 | acetyl-Coenzyme A acyltransferase 1 (peroxisomal 3-oxoacyl-Coenzyme A thiolase) |
| 47 | ACLY | ATP citrate lyase |
| 2539 | G6PD | glucose-6-phosphate dehydrogenase |
| 3162 | HMOX1 | heme oxygenase (decycling) 1 |
| 3417 | IDH1 | isocitrate dehydrogenase 1 (NADP+), soluble |
| 3939 | LDHA | lactate dehydrogenase A |
| 4190 | MDH1 | malate dehydrogenase 1, NAD (soluble) |
| 5207 | PFKFB1 | 6-phosphofructo-2-kinase/fructose-2,6-biphosphatase 1 |
| 23600 | AMACR | alpha-methylacyl-CoA racemase |
| 2538 | G6PC | glucose-6-phosphatase, catalytic (glycogen storage disease type I, von Gierke disease) |
| 3795 | KHK | ketohexokinase (fructokinase) |
| 3157 | HMGCS1 | 3-hydroxy-3-methylglutatyl-Coenzyme A synthase 1 (soluble) |
| 5214 | PFKP | phosphofructokinase, platelet |
| 4942 | OAT | ornithine aminotransferase (gyrate atrophy) |
| 2342 | FNTB | farnesyltransferase, CAAX box, beta |
| 7086 | TKT | transketolase (Wernicke-Korsakoff syndrome) |
| 1891 | ECH1 | enoyl Coenzyme A hydratase 1, peroxisomal |
| 3294 | HSD17B2 | hydroxysteroid (17-beta) dehydrogenase 2 |
| 9415 | FADS2 | fatty acid desaturase 2 |
| 6888 | TALDO1 | transaldolase 1 |
| 6799 | SULT1A2 | sulfotransferase family, cytosolic, 1A, phenol-preferring, member 2 |
| 2224 | FDPS | farnesyl diphosphate synthase (farnesyl pyrophosphate synthetase, dimethylallyltranstransferase, geranyltranstransferase) |
| 3992 | FADS1 | fatty acid desaturase 1 |
| 1666 | DECR1 | 2,4-dienoyl CoA reductase 1, mitochondrial |
| 6307 | SC4MOL | sterol-C4-methyl oxidase-like |
| 10449 | ACAA2 | acetyl-Coenzyme A acyltransferase 2 (mitochondrial 3-oxoacyl-Coenzyme A thiolase) |
| 6319 | SCD | stearoyl-CoA desaturase (delta-9-desaturase) |

human subset 2 (hl)
2260; 4842; 8655; 10060; 11244; 51691
human subset 3 (hl)
19; 51; 90; 100; 207; 210; 211; 535; 571; 644; 847; 1026; 1231; 1385; 1386; 1432; 1528; 1535; 1543; 1571; 1655; 2052; 2145; 2237; 2247; 2260; 2308; 2339; 2341; 2343;
2542; 2597; 2646; 2766; 2803; 2819; 2821; 3043; 3091; 3163; 3172; 3184; 3251; 3265; 3283; 3292; 3295; 3303; 3422; 3576; 3596; 3897; 3940; 3945; 3952; 3992; 3995;
4191; 4654; 4780; 4842; 4921; 4943; 5209; 5211; 5213; 5468; 5594; 5595; 6309; 6342; 6647; 6667; 6670; 6817; 6818; 6889; 7001; 7015; 7040; 7041; 7052; 7124; 7422;
7439; 7702; 8083; 9415; 9941; 9942; 9943; 10060; 10599; 10654; 10805; 54658; 158222; 283226; 338025; 441769
chimpanzee subset 1 (hl)
460268; 454672; 470195; 459271; 461953; 454703; 470335; 461892; 450266; 450803; 452971; 460443; 454269; 454013; 451243; 455414; 450676
chimpanzee subset 2 (hl)
467139; 464368
chimpanzee subset 3 (hl)
454895; 470565; 453202; 464675; 460416; 454685; 458500; 463366; 451116; 462643; 468580; 468068; 450857; 457777; 454697; 451242; 472929; 451599; 451783;
459107; 462453; 460264; 455941; 450978; 452958; 453880; 458269; 461216; 466302; 454689; 462019; 450263; 471205; 449638; 451243; 451246; 463484; 451057;
459769; 467139; 457467; 450287; 458602; 451863; 460178; 454037; 451624; 456862; 451939; 459750; 454013; 468740; 471443; 456060; 467962; 466630; 451017;
470791; 465331; 457350; 460032

human subset 1 (ez)

| | | |
|---|---|---|
| 216 | ALDH1A1 | aldehyde dehydrogenase 1 family, member A1 |
| 1543 | CYP1A1 | cytochrome P450, family 1, subfamily A, polypeptide 1 |
| 1728 | NQO1 | NAD(P)H dehydrogenase, quinone 1 |
| 3659 | IRF1 | interferon regulatory factor 1 |
| 4501 | MT1X | metallothionein 1X |
| 6890 | TAP1 | transporter 1, ATP-binding cassette, sub-family B (MDR/TAP) |
| 581 | BAX | BCL2-associated X protein |
| 5698 | PSMB9 | proteasome (prosome, macropain) subunit, beta type, 9 (large multifunctional peptidase 2) |
| 5696 | PSMB8 | proteasome (prosome, macropain) subunit, beta type, 8 (large multifunctional peptidase 7) |
| 1727 | CYB5R3 | cytochrome b5 reductase 3 |
| 10993 | SDS | serine dehydratase |
| 6772 | STAT1 | signal transducer and activator of transcription 1, 91 kDa |
| 27232 | GNMT | glycine N-methyltransferase |
| 2729 | GCLC | glutamate-cysteine ligase, catalytic subunit |
| 2052 | EPHX1 | epoxide hydrolase 1, microsomal (xenobiotic) |
| 4255 | MGMT | O-6-methylguanine-DNA methyltransferase |
| 900 | CCNG1 | cyclin G1 |
| 1410 | CRYAB | crystallin, alpha B |
| 972 | CD74 | CD74 antigen (invariant polypeptide of major histocompatibility complex, class II antigen-associated) |
| 22977 | AKR7A3 | aldo-keto reductase family 7, member A3 (aflatoxin aldehyde reductase) |
| 1634 | DCN | decorin |
| 7298 | TYMS | thymidylate synthetase |
| 6223 | RPS19 | ribosomal protein S19 |
| 199 | AIF1 | allograft inflammatory factor 1 |
| 9518 | GDF15 | growth differentiation factor 15 |
| 7832 | BTG2 | BTG family, member 2 |
| 516 | ATP5G1 | ATP synthase, H+ transporting, mitochondrial F0 complex, subunit c (subunit 9), isoform 1 |
| 1520 | CTSS | cathepsin S |
| 10095 | ARPC1B | actin related protein ⅔ complex, subunit 1B, 41 kDa |
| 302 | ANXA2 | annexin A2 |
| 595 | CCND1 | cyclin D1 |
| 2621 | GAS6 | growth arrest-specific 6 |
| 4942 | OAT | ornithine aminotransferase (gyrate atrophy) |
| 388 | RHOB | ras homolog gene family, member B |
| 80273 | GRPEL1 | GrpE-like 1, mitochondrial (E. coli) |
| 635 | BHMT | betaine-homocysteine methyltransferase |
| 6906 | SERPINA7 | serpin peptidase inhibitor, clade A (alpha-1 antiproteinase, antitrypsin), member 7 |
| 7431 | VIM | vimentin |
| 6232 | RPS27 | ribosomal protein S27 (metallopanstimulin 1) |
| 3084 | NRG1 | neuregulin 1 |
| 54578 | UGT1A6 | UDP glucuronosyltransferase 1 family, polypeptide A6 |
| 1026 | CDKN1A | cyclin-dependent kinase inhibitor 1A (p21, Cip1) |
| 7076 | TIMP1 | TIMP metallopeptidase inhibitor 1 |
| 2212 | FCGR2A | Fc fragment of IgG, low affinity IIa, receptor (CD32) |
| 2936 | GSR | glutathione reductase |
| 27165 | GLS2 | glutaminase 2 (liver, mitochondrial) |
| 10669 | CGREF1 | cell growth regulator with EF-hand domain 1 |
| 3627 | CXCL10 | chemokine (C—X—C motif) ligand 10 |
| 3122 | HLA-DRA | major histocompatibility complex, class II, DR alpha |
| 3127 | HLA-DRB5 | major histocompatibility complex, class II, DR beta 5 |
| 3324 | HSPCAL3 | heat shock 90 kDa protein 1, alpha-like 3 |
| 51382 | ATP6V1D | ATPase, H+ transporting, lysosomal 34 kDa, V1 subunit D |
| 3120 | HLA-DQB2 | major histocompatibility complex, class II, DQ beta 2 |
| 4193 | MDM2 | Mdm2, transformed 3T3 cell double minute 2, p53 binding protein (mouse) |

human subset 2 (ez)
350; 398; 596; 597; 598; 634; 637; 672; 801; 1019; 1020; 1021; 1026; 1027; 1029; 1032; 1147; 1316; 1385; 1387; 1409; 1410; 1431; 1457; 1460; 1499; 1508; 1647; 1655;
1956; 2006; 2033; 2071; 2099; 2200; 2308; 2309; 2316; 2335; 2580; 2810; 2885; 2959; 3055; 3065; 3091; 3108; 3109; 3123; 3127; 3303; 3315; 3429; 3661; 3732; 3827;
3958; 3984; 4089; 4155; 4170; 4193; 4194; 4267; 4282; 4286; 4303; 4582; 4605; 4609; 4616; 4790; 5111; 5147; 5245; 5292; 5295; 5300; 5320; 5327; 5335; 5340; 5366;
5371; 5425; 5430; 5433; 5469; 5524; 5566; 5684; 5699; 5716; 5925; 5933; 5934; 5970; 5981; 6125; 6135; 6275; 6277; 6281; 6317; 6318; 6418; 6441; 6464; 6597; 6667;
6714; 6772; 6773; 6774; 6850; 6872; 6892; 6908; 7040; 7057; 7067; 7083; 7124; 7132; 7133; 7157; 7161; 7251; 7301; 7317; 7329; 7341; 7373; 7416; 7421; 7514; 7528; 7534; 7709; 7874; 8202; 8290; 8291;
8317; 8452; 8554; 8648; 8650; 8655; 8717; 8840; 8850; 8900; 8996; 9063; 9111; 9150; 9261; 9337; 9412; 9439; 9463; 9466; 9774; 9978; 9988; 10017; 10097; 10109; 10270; 10379; 10399; 10434; 10461;
10524; 10589; 10912; 10923; 10971; 23118; 23582; 23650; 23741; 23762; 25766; 25792; 27040; 27085; 27113; 27257; 29124; 29883; 30851; 50650; 51100; 51191; 51365; 51535; 51729; 51762; 54205;
55250; 55388; 56647; 56654; 64112; 64219; 64375; 64780; 79902; 85415; 121504; 151531; 168544; 255488
human subset 3 (ez)
2; 10; 25; 43; 60; 71; 102; 124; 125; 142; 147; 196; 197; 207; 217; 218; 238; 246; 291; 293; 301; 303; 304; 305; 308; 309; 324; 328; 332; 350; 351; 355; 356; 367; 374; 387;
388; 389; 391; 396; 398; 408; 409; 410; 467; 471; 472; 506; 515; 517; 518; 520; 522; 523; 545; 558; 567; 571; 572; 578; 581; 595; 596; 597; 598; 602; 613; 631; 632; 633;
634; 637; 638; 640; 649; 652; 654; 660; 665; 667; 672; 685; 694; 712; 728; 801; 811; 817; 818; 823; 831; 834; 835; 836; 839; 840; 841; 842; 847; 860; 861; 864; 871; 890;
891; 892; 894; 896; 898; 900; 901; 912; 920; 940; 945; 958; 959; 960; 967; 972; 983; 997; 999; 1003; 1017; 1019; 1020; 1021; 1025; 1026; 1027; 1028; 1029; 1030; 1031;
1032; 1033; 1045; 1050; 1051; 1062; 1072; 1080; 1129; 1147; 1191; 1231; 1232; 1233; 1234; 1277; 1278; 1280; 1316; 1385; 1386; 1387; 1401; 1409; 1410; 1415; 1420;
1427; 1431; 1432; 1434; 1435; 1457; 1460; 1462; 1466; 1471; 1473; 1491; 1495; 1499; 1504; 1508; 1509; 1512; 1513; 1514; 1528; 1543; 1544; 1545; 1555; 1559; 1571;
1576; 1586; 1616; 1639; 1647; 1649; 1655; 1674; 1718; 1719; 1727; 1728; 1742; 1803; 1806; 1832; 1839; 1869; 1874; 1890; 1950; 1956; 1958; 1977; 1993; 1994; 2006;
2017; 2033; 2053; 2059; 2064; 2065; 2066; 2067; 2068; 2099; 2100; 2113; 2125; 2147; 2149; 2168; 2176; 2200; 2208; 2209; 2210; 2211; 2213; 2214; 2215; 2237; 2247;
2260; 2261; 2264; 2316; 2335; 2353; 2475; 2520; 2551; 2580; 2597; 2626; 2643; 2670; 2678; 2688; 2689; 2706; 2720; 2730; 2735; 2744; 2810; 2833; 2876; 2879; 2885;
2908; 2923; 2932; 2938; 2944; 2950; 2952; 2961; 2969; 3055; 3065; 3066; 3067; 3087; 3091; 3094; 3105; 3106; 3108; 3109; 3112; 3113; 3115; 3117; 3119; 3122; 3123;
3124; 3125; 3126; 3127; 3131; 3133; 3134; 3135; 3159; 3162; 3190; 3192; 3219; 3224; 3265; 3276; 3297; 3299; 3303; 3315; 3316; 3320; 3322; 3323; 3325; 3381; 3394;
3429; 3438; 3439; 3458; 3459; 3479; 3480; 3486; 3490; 3551; 3565; 3567; 3569; 3574; 3576; 3586; 3587; 3592; 3593; 3596; 3606; 3621; 3630; 3636; 3638; 3654; 3659;
3660; 3661; 3663; 3665; 3673; 3688; 3716; 3717; 3718; 3725; 3728; 3732; 3791; 3815; 3827; 3836; 3845; 3848; 3856; 3875; 3932; 3952; 3958; 3964; 3984; 3985; 4035;
4049; 4050; 4058; 4060; 4067; 4070; 4085; 4088; 4133; 4152; 4155; 4170; 4172; 4174; 4192; 4193; 4194; 4216; 4221; 4233; 4237; 4255; 4257; 4261; 4282; 4283; 4292;
4297; 4312; 4313; 4314; 4316; 4317; 4318; 4319; 4320; 4323; 4363; 4436; 4478; 4489; 4493; 4494; 4495; 4496; 4499; 4500; 4502; 4512; 4524; 4599; 4605; 4609; 4613;
4616; 4666; 4683; 4687; 4739; 4760; 4780; 4790; 4791; 4795; 4800; 4828; 4831; 4835; 4843; 4899; 4915; 4927; 4943; 5008; 5045; 5058; 5062; 5093; 5111; 5170; 5175;
5178; 5241; 5243; 5245; 5292; 5295; 5300; 5320; 5327; 5329; 5335; 5339; 5340; 5347; 5366; 5371; 5451; 5465; 5468; 5493; 5521; 5524; 5536; 5562; 5566; 5578; 5580;
5581; 5583; 5585; 5591; 5594; 5595; 5597; 5599; 5600; 5610; 5617; 5621; 5657; 5684; 5686; 5689; 5693; 5695; 5696; 5698; 5699; 5715; 5716; 5720; 5721; 5728; 5743;
5747; 5770; 5771; 5777; 5781; 5879; 5903; 5906; 5915; 5916; 5925; 5933; 5934; 5970; 5981; 5982; 5983; 5984; 5985; 6059; 6060; 6093; 6125; 6135; 6170; 6275; 6277;
6281; 6294; 6297; 6302; 6303; 6317; 6318; 6319; 6346; 6347; 6348; 6351; 6352; 6354; 6355; 6356; 6361; 6366; 6367; 6373; 6375; 6376; 6387; 6401; 6403; 6418; 6427;
6441; 6464; 6470; 6573; 6590; 6597; 6598; 6609; 6622; 6640; 6647; 6648; 6667; 6670; 6688; 6696; 6708; 6709; 6714; 6722; 6750; 6753; 6772; 6773; 6774; 6775; 6776;
6777; 6778; 6817; 6850; 6872; 6890; 6891; 6892; 6929; 6996; 7015; 7039; 7040; 7048; 7057; 7067; 7076; 7077; 7078; 7079; 7083; 7097; 7099; 7124; 7126; 7132; 7133;
7153; 7157; 7159; 7161; 7184; 7251; 7272; 7273; 7291; 7295; 7296; 7301; 7302; 7317; 7322; 7323; 7329; 7341; 7361; 7364; 7373; 7378; 7402; 7412; 7415; 7416; 7417;
7421; 7422; 7424; 7428; 7450; 7454; 7465; 7471; 7486; 7494; 7514; 7515; 7517; 7525; 7528; 7534; 7709; 7852; 7874; 7916; 8061; 8091; 8202; 8290; 8295; 8312; 8315;
8379; 8451; 8452; 8467; 8473; 8536; 8554; 8574; 8626; 8648; 8650; 8651; 8714; 8717; 8743; 8795; 8797; 8840; 8850; 8900; 9021; 9063; 9111; 9134; 9170; 9252; 9261;
9337; 9338; 9349; 9365; 9401; 9402; 9466; 9540; 9542; 9545; 9601; 9603; 9774; 9836; 9927; 9978; 9988; 10017; 10018; 10068; 10093; 10094; 10096; 10097; 10109;
10140; 10155; 10270; 10288; 10290; 10379; 10399; 10403; 10413; 10417; 10434; 10461; 10524; 10668; 10766; 10848; 10859; 10912; 10953; 10971; 11135; 11140; 11177; 11186; 11200; 11275; 22943; 22974;
23113; 23368; 23433; 23464; 23582; 23650; 23741; 23762; 25792; 25855; 25937; 26133; 26281; 26574; 27018; 27040; 27085; 27113; 27250; 29108; 29124; 29883; 30851; 50650; 50848; 51079; 51100; 51176;
51191; 51365; 51535; 51588; 51729; 54106; 54205; 54434; 54476; 54575; 54577; 54600; 54657; 54658; 54766; 55503; 55856; 55929; 56302; 56647; 56904; 63970; 64112; 64219; 64375; 64395; 64780; 79753;
79902; 80205; 80309; 80781; 84062; 84168; 85415; 85480;121504; 140691; 151531; 168544; 171017; 221468; 246181; 246182; 246778; 255488; 267012; 442219
chimpanzee subset 1 (ez)
464529; 468012; 462596; 458883; 470721; 462703; 462776; 457777; 462243; 462191; 469197; 452120; 456065; 468770; 469648; 455195; 457277; 463568; 453487;
450803; 459054; 471135; 465791; 457344; 450194; 451998; 470336; 461242; 462591; 462594; 452979; 462595; 470901
chimpanzee subset 2 (ez)
458156; 458635; 449497; 453100; 452025; 463891; 463534; 465021; 450673; 450269; 467891; 458589; 455850; 463993; 456929; 458862; 470526; 463079; 461048;
458163; 464259; 452958; 449592; 462594; 462594; 463488; 453250; 466498; 460916; 463466; 468537; 457274; 457658; 465979; 473659; 458262; 461406; 458081;
455205; 462656; 461836; 456587; 472748; 453736; 472654; 470528; 454629; 464784; 465803; 452715; 467978; 466665; 461174; 457025; 457320; 457306; 455459;
466127; 457351; 451939; 458229; 470721; 465701; 456060; 453319; 468342; 466934; 457968; 451065; 453352; 465601; 467865; 459882; 471632; 451858; 472830;
453907; 458308; 469380; 459983; 467715; 453014; 454154; 472862; 460219; 467260; 455403; 470714; 462213; 465356; 472124; 456370; 458860; 472434; 467685;
462344; 459492; 451329; 451337; 464585; 470302; 453373; 451615; 464902; 464361; 469081; 460466; 470894; 451837; 465307; 465522; 455378; 450814; 464882;
453122; 451979; 454876; 465382; 470566
chimpanzee subset 3 (ez)
464802; 450133; 454959; 453472; 461396; 457785; 460912; 453202; 452252; 468186; 459126; 472953; 461466; 462198; 461999; 465200; 466149; 469584; 465680;
461251; 459054; 457098; 454450; 458946; 459925; 451999; 457109; 451943; 470584; 458487; 471061; 460745; 456057; 450170; 458500; 451291; 462616; 458156;
473829; 458635; 470232; 463991; 464042; 464069; 449497; 461250; 452121; 468931; 453100; 455756; 461445; 451317; 462348; 451519; 463804; 450745; 460110;
456457; 451116; 472017; 466899; 461467; 461835; 462689; 455914; 462243; 461229; 469524; 450124; 470617; 473802; 451969; 462191; 450481; 452025; 463891;
463534; 465021; 455931; 469969; 461413; 466666; 463674; 463758; 450673; 464075; 470810; 470781; 450128; 463542; 450269; 467891; 457428; 458589; 460909;
455850; 462643; 458318; 457127; 461766; 452089; 456939; 462100; 463993; 453574; 457278; 468580; 453745; 456052; 450626; 450857; 462613; 459332; 456929;
452017; 470657; 461773; 458883; 468012; 468444; 459689; 457047; 462420; 458184; 454158; 458941; 462097; 461391; 465023; 455668; 451391; 458862; 464074;
473379; 454636; 451980; 459919; 450179; 456124; 463079; 454896; 451158; 471510; 465257; 456434; 457457; 451242; 461067; 462301; 453047; 468262; 461048;
451783; 454741; 470168; 467220; 460254; 470606; 471681; 453387; 470895; 451717; 464098; 463472; 458163; 464259; 462950; 453425; 450612; 452958; 462040;
462532; 450202; 449592; 471977; 462598; 462599; 471974; 462591; 462594; 462594; 455140; 462529; 463179; 462624; 470195; 464554; 457867; 466302; 463488;
451544; 471249; 454294; 453250; 473197; 449517; 453676; 463396; 472749; 449565; 463288; 464245; 471205; 469657; 451580; 460816; 471723; 450954; 451403;
463839; 465935; 461629; 450927; 450396; 456911; 455848; 456893; 454667; 466498; 461315; 461316; 460916; 460631; 473387; 449638; 469155; 463466; 452006;
453350; 452119; 472771; 461661; 455418; 457274; 462763; 458790; 457658; 463119; 463671; 473380; 453914; 471200; 471095; 451587; 451510; 454094; 451511;
451509; 458701; 459215; 454105; 471221; 807866; 458565; 458262; 459043; 452434; 464278; 459769; 461406; 462395; 455026; 472513; 453652; 451439; 460965;
459300; 458081; 454766; 469030; 451502; 455205; 462656; 461836; 456587; 472748; 450103; 453991; 453736; 460178; 453893; 462170; 464784; 456150; 471564;
459203; 464165; 454037; 450445; 459155; 462468; 458076; 473258; 462596; 465187; 465803; 461963; 466142; 464422; 469970; 455504; 467135; 463253; 459477;
469408; 451935; 452715; 467978; 466665; 461174; 463467; 452533; 460918; 461523; 455326; 457025; 465828; 457320; 457306; 452024; 465538; 455459; 450676;
455004; 454579; 468209; 468208; 467985; 467984; 469572; 454115; 450191; 466127; 457351; 458702; 450990; 461375; 449634; 451939; 459750; 461366; 458229;
462709; 471035; 458807; 470721; 459834; 454681; 454679; 452005; 454013; 465701; 471978; 468650; 452188; 471443; 470399; 456060; 460243; 453319; 454928;
458784; 460176; 468342; 471325; 466934; 457968; 452187; 451065; 462844; 459783; 450779; 467112; 453352; 465601; 462108; 467865; 459882; 471218; 472361;
471632; 450537; 451858; 471358; 466440; 451871; 472732; 458735; 456094; 453191; 455322; 472830; 450130; 453907; 471965; 458308; 463564; 452694; 459983; 461517; 465702; 460153; 467715; 469196;
453014; 455128; 454154; 460852; 472862; 460219; 467260; 455403; 470714; 464292; 455878; 462213; 454619; 464469; 456370; 471648; 468721; 463298; 458860; 472434; 467685; 459487; 460155; 468406;
456353; 462344; 455320; 451506; 459492; 451329; 452916; 458865; 469007; 464585; 458274; 470302; 458813; 455704; 452855; 460392; 458733; 461590; 450465; 458157; 463192; 453193; 458820; 453373;
451615; 464902; 458196; 454601; 465780; 464361; 450735; 454067; 469081; 460466; 469555; 455877; 461425; 470894; 451837; 465307; 465522; 460032; 451538; 450814; 464781; 453122; 451979; 456759;
454876; 460119; 465382; 467799; 470566; 451790; 462463

human subset 1 (hg)

| | | |
|---|---|---|
| 847 | CAT | catalase |
| 2538 | G6PC | glucose-6-phosphatase, catalytic (glycogen storage disease type I, von Gierke disease) |
| 3399 | ID3 | inhibitor of DNA binding 3, dominant negative helix-loop-helix protein |
| 249 | ALPL | alkaline phosphatase, liver/bone/kidney |
| 5105 | PCK1 | phosphoenolpyruvate carboxykinase 1 (soluble) |
| 2328 | FMO3 | flavin containing monooxygenase 3 |
| 2326 | FMO1 | flavin containing monooxygenase 1 |
| 54578 | UGT1A6 | UDP glucuronosyltransferase 1 family, polypeptide A6 |
| 1066 | CES1 | carboxylesterase 1 (monocyte/macrophage serine esterase 1) |
| 3557 | IL1RN | interleukin 1 receptor antagonist |
| 1515 | CTSL2 | cathepsin L2 |
| 4599 | MX1 | myxovirus (influenza virus) resistance 1, interferon-inducible protein p78 (mouse) |
| 183 | AGT | angiotensinogen (serpin peptidase inhibitor, clade A, member 8) |
| 27294 | DHDH | dihydrodiol dehydrogenase (dimeric) |
| 4501 | MT1X | metallothionein 1X |
| 7422 | VEGF | vascular endothelial growth factor |
| 7076 | TIMP1 | TIMP metallopeptidase inhibitor 1 |
| 2335 | FN1 | fibronectin 1 |
| 3576 | IL8 | interleukin 8 |
| 4318 | MMP9 | matrix metallopeptidase 9 (gelatinase B, 92 kDa gelatinase, 92 kDa type IV collagenase) |
| 6799 | SULT1A2 | sulfotransferase family, cytosolic, 1A, phenol-preferring, member 2 |
| 23471 | TRAM1 | translocation associated membrane protein 1 |
| 6746 | SSR2 | signal sequence receptor, beta (translocon-associated protein beta) |
| 6745 | SSR1 | signal sequence receptor, alpha (translocon-associated protein alpha) |
| 57007 | CMKOR1 | chemokine orphan receptor 1 |
| 3383 | ICAM1 | intercellular adhesion molecule 1 (CD54), human rhinovirus receptor |
| 6347 | CCL2 | chemokine (C-C motif) ligand 2 |
| 3569 | IL6 | interleukin 6 (interferon, beta 2) |
| 29927 | SEC61A1 | Sec61 alpha 1 subunit (S. cerevisiae) |
| 1565 | CYP2D6 | cytochrome P450, family 2, subfamily D, polypeptide 6 |
| 1557 | CYP2C19 | cytochrome P450, family 2, subfamily C, polypeptide 19 |
| 1571 | CYP2E1 | cytochrome P450, family 2, subfamily E, polypeptide 1 |
| 1555 | CYP2B6 | cytochrome P450, family 2, subfamily B, polypeptide 6 |
| 7086 | TKT | transketolase (Wernicke-Korsakoff syndrome) |
| 5950 | RBP4 | retinol binding protein 4, plasma |

human subset 2 (hg)
185; 186; 2934; 2990; 3371; 3486; 3559; 3561; 3570; 3572; 3577; 3579; 3675; 3676; 3678; 3683; 3684; 3689; 3694; 3695; 3959; 4018; 4052; 4099; 4142; 4147; 4224; 4225;
4318; 4478; 4617; 4653; 4654; 4790; 4856; 5068; 5079; 5310; 5430; 5447; 5549; 5553; 5744; 5781; 5858; 5970; 6351; 6374; 6382; 6383; 6387; 6423; 6720; 6863; 6925;
6929; 7045; 7052; 7057; 7058; 7078; 7220; 7222; 7223; 7224; 7225; 7253; 7276; 7280; 7356; 7428; 7430; 7448; 7850; 8190; 8290; 8434; 8516; 8658; 8828; 8829; 8841;
9510; 9612; 11036; 23549; 53371; 57113; 90070; 116844; 164656
human subset 3 (hg)
2; 25; 27; 30; 51; 102; 118; 125; 126; 133; 134; 135; 136; 142; 176; 177; 185; 186; 207; 210; 213; 217; 238; 248; 250; 251; 259; 283; 284; 285; 325; 338; 348; 354; 355; 356;
367; 374; 383; 387; 466; 468; 472; 535; 595; 596; 598; 624; 627; 632; 641; 649; 652; 660; 672; 673; 685; 712; 718; 799; 819; 820; 821; 836; 841; 847; 857; 860; 861; 871;
904; 929; 940; 941; 943; 944; 947; 952; 958; 959; 960; 962; 965; 967; 968; 972; 984; 985; 998; 999; 1017; 1025; 1027; 1050; 1051; 1113; 1114; 1118; 1137; 1215; 1230;
1231; 1232; 1234; 1238; 1277; 1278; 1280; 1281; 1282; 1284; 1291; 1292; 1294; 1305; 1356; 1387; 1398; 1401; 1432; 1435; 1437; 1440; 1445; 1466; 1470; 1490; 1499;
1508; 1509; 1514; 1543; 1544; 1546; 1548; 1549; 1554; 1555; 1556; 1557; 1558; 1559; 1562; 1564; 1565; 1568; 1571; 1574; 1576; 1577; 1580; 1588; 1604; 1616; 1634;
1636; 1649; 1655; 1728; 1803; 1813; 1814; 1827; 1843; 1890; 1903; 1906; 1909; 1910; 1915; 1942; 1950; 1956; 1969; 1977; 1978; 1981; 1991; 1994; 2000; 2006; 2022;
2034; 2050; 2052; 2056; 2064; 2091; 2099; 2101; 2113; 2145; 2147; 2150; 2152; 2160; 2162; 2165; 2185; 2192; 2194; 2199; 2208; 2244; 2247; 2250; 2260; 2266; 2277;
2308; 2321; 2323; 2324; 2326; 2327; 2328; 2329; 2330; 2335; 2337; 2353; 2354; 2358; 2475; 2521; 2532; 2542; 2668; 2673; 2678; 2688; 2771; 2778; 2784; 2817; 2826;
2876; 2885; 2902; 2908; 2919; 2932; 2934; 2936; 2938; 2944; 2950; 2952; 2971; 2990; 3002; 3082; 3091; 3099; 3162; 3169; 3170; 3171; 3184; 3274; 3297; 3320; 3329;
3336; 3339; 3352; 3371; 3383; 3384; 3385; 3397; 3398; 3439; 3456; 3458; 3479; 3480; 3481; 3484; 3485; 3486; 3488; 3490; 3547; 3551; 3552; 3553; 3554; 3557; 3558;
3559; 3561; 3562; 3565; 3569; 3570; 3572; 3574; 3575; 3576; 3577; 3579; 3581; 3586; 3587; 3589; 3593; 3596; 3605; 3606; 3624; 3627; 3630; 3643; 3655; 3659; 3667;
3673; 3675; 3676; 3678; 3680; 3683; 3684; 3685; 3687; 3688; 3689; 3690; 3691; 3693; 3694; 3695; 3717; 3725; 3727; 3778; 3791; 3815; 3827; 3914; 3931; 3932; 3934;
3952; 3958; 3976; 4012; 4018; 4035; 4050; 4052; 4055; 4087; 4088; 4089; 4099; 4142; 4152; 4170; 4179; 4224; 4225; 4233; 4254; 4261; 4270; 4282; 4283; 4288; 4306;
4312; 4313; 4314; 4316; 4317; 4318; 4319; 4320; 4323; 4360; 4478; 4489; 4493; 4494; 4495; 4496; 4499; 4500; 4502; 4513; 4582; 4600; 4609; 4615; 4653; 4654; 4665;
4681; 4687; 4690; 4772; 4773; 4790; 4791; 4811; 4830; 4843; 4846; 4851; 4856; 4868; 4878; 4893; 4921; 4938; 4982; 4988; 5008; 5020; 5054; 5068; 5074; 5079; 5154;
5155; 5175; 5176; 5189; 5196; 5228; 5268; 5292; 5303; 5310; 5321; 5327; 5328; 5329; 5337; 5338; 5340; 5447; 5465; 5467; 5468; 5473; 5550; 5553; 5563; 5578; 5579;
5580; 5581; 5592; 5594; 5595; 5599; 5603; 5604; 5610; 5617; 5624; 5700; 5728; 5732; 5734; 5743; 5744; 5745; 5747; 5764; 5771; 5781; 5788; 5829; 5830; 5858; 5879;
5887; 5921; 5925; 5934; 5970; 5971; 5972; 5979; 6037; 6095; 6198; 6261; 6280; 6287; 6288; 6289; 6346; 6347; 6348; 6351; 6352; 6354; 6355; 6356; 6357; 6361; 6364;
6367; 6372; 6374; 6376; 6382; 6383; 6387; 6401; 6402; 6403; 6405; 6416; 6423; 6446; 6464; 6520; 6564; 6574; 6623; 6647; 6648; 6667; 6670; 6678; 6714; 6720; 6722;
6729; 6734; 6770; 6772; 6773; 6774; 6778; 6817; 6818; 6863; 6929; 7010; 7013; 7018; 7020; 7033; 7040; 7042; 7044; 7045; 7046; 7052; 7056; 7057; 7058; 7066; 7073;
7075; 7076; 7077; 7078; 7079; 7097; 7099; 7100; 7124; 7132; 7133; 7157; 7186; 7187; 7188; 7189; 7253; 7276; 7280; 7292; 7295; 7356; 7361; 7364; 7376; 7412; 7421; 7422; 7423; 7424; 7428; 7430; 7432;
7448; 7450; 7850; 7852; 7869; 8061; 8190; 8431; 8434; 8516; 8531; 8570; 8600; 8651; 8658; 8660; 8718; 8809; 8828; 8829; 8837; 8856; 8870; 8877; 8900; 8936; 9021; 9112; 9170; 9232; 9370; 9407; 9507;
9510; 9672; 9966; 9970; 9971; 10062; 10096; 10097; 10544; 10575; 10664; 10850; 10890; 10952; 11036; 22795; 23428; 23480; 23491; 23516; 23549; 23566; 26525; 27018; 27087; 27189; 27230; 27236; 29108;
29127; 29949; 30835; 51247; 54567; 54575; 54577; 54600; 54657; 54658; 55176; 55303; 55729; 56547; 56848; 56895; 57085; 59272; 60675; 80309; 80351; 80781; 83939; 84432; 90070; 116844; 164656;
282616; 282618; 284217; 391533
chimpanzee subset 1 (hg)
451116; 454703; 456618; 457505; 459501; 458565; 450104; 456194; 471205; 454013; 464227; 457379; 462417; 460122; 450196; 463288; 470228; 450857; 456052; 460443; 450617
chimpanzee subset 2 (hg)
460762; 463443; 463396; 450884; 469506; 461866; 470645; 470644; 455111; 470596; 451958; 458607; 451934; 454930; 463116; 459143; 469075; 472176; 462745;
468515; 467081; 457511; 451057; 461406; 459349; 454340; 463481; 469649; 451190; 473393; 467135; 465371; 466665; 455004; 471203; 464298; 461556; 455432;
468650; 462081; 453319; 463138; 458784; 460747; 471294; 452544; 466634; 470455; 456048; 450323; 463982; 460111; 450397; 470057; 467150; 459964; 461240;
451963
chimpanzee subset 3 (hg)
464802; 457552; 460268; 454895; 453472; 461396; 451021; 457638; 458714; 455009; 457785; 453629; 462584; 460762; 453202; 464675; 461260; 452252; 459126;
470679; 464334; 457427; 459058; 466149; 469584; 465680; 461251; 462999; 452429; 468334; 454685; 458156; 467547; 453650; 464042; 449497; 463781; 461250;
455644; 472445; 470816; 462330; 460110; 451116; 463670; 472017; 466899; 462118; 470617; 470892; 457960; 464686; 457692; 473802; 457437; 457154; 451969;
468440; 462191; 456609; 455931; 469969; 470034; 457641; 469999; 470810; 450128; 460298; 463542; 459815; 460347; 450508; 467891; 457428; 462643; 457127;
471627; 454641; 452089; 458144; 463003; 463993; 453745; 456052; 450909; 450626; 470228; 450857; 463579; 453433; 462613; 452120; 454782; 452017; 468012;
459689; 451553; 470885; 471742; 458941; 464580; 462437; 452613; 472057; 457356; 461391; 460888; 468637; 455668; 459204; 463610; 457777; 463609; 454636;
456031; 463079; 466651; 454697; 451158; 461800; 457038; 462413; 464073; 458908; 454986; 456434; 461558; 471190; 461560; 456206; 462339; 457505; 469472;
453047; 456127; 451599; 471566; 459296; 470168; 469986; 471681; 470895; 451717; 463443; 463503; 452958; 470195; 456143; 461216; 471750; 459852; 452179;
456606; 469219; 450196; 469912; 459009; 473197; 473183; 449517; 453676; 466318; 463395; 459932; 463396; 459933; 465861; 472749; 470485; 450200; 459460;
459501; 461472; 450884; 471626; 449565; 463288; 469506; 461866; 464245; 461922; 471205; 470645; 470644; 469657; 451580; 471723; 472029; 463371; 461242;
450954; 455649; 459742; 455111; 470596; 451958; 470789; 459807; 454070; 450396; 458607; 468295; 451934; 456893; 461315; 461316; 460916; 469668; 464764;
449638; 461960; 463116; 452006; 459143; 451778; 455407; 468537; 469075; 472176; 455418; 457274; 469663; 462745; 468515; 463671; 452114; 453914; 471200;
450866; 461533; 451510; 454094; 451511; 451509; 458701; 450333; 454105; 471221; 807864; 458564; 460269; 457511; 451057; 455485; 469972; 461406; 455134;
455026; 468841; 469801; 457467; 464351; 472165; 470020; 472467; 459349; 452094; 458844; 454766; 463532; 453041; 468566; 462656; 454340; 472748; 450103;
470996; 454454; 463481; 460178; 462903; 451190; 459203; 454037; 450445; 462644; 453526; 459155; 462468; 459593; 466142; 467459; 450195; 473393; 464422;
463759; 455504; 467135; 457607; 451812; 465371; 463253; 473012; 461762; 452715; 467978; 466665; 456119; 469651; 450415; 457998; 466459; 455004; 454579;
468209; 468208; 467985; 459991; 467984; 471204; 471203; 454115; 464298; 450191; 460387; 455065; 461556; 463011; 457351; 451271; 467307; 459498; 450574;
449634; 451939; 459750; 471709; 458229; 462709; 452858; 466844; 470721; 452005; 454013; 468650; 465028; 472793; 460710; 456060; 457742; 457780; 462081; 472992; 469898; 453319; 463138; 450776;
469308; 454928; 458784; 460176; 471325; 457762; 466934; 457968; 464876; 453178; 457711; 450474; 469586; 450779; 466634; 471218; 451858; 466648; 471358; 472164; 470455; 450130; 456048; 450323;
465289; 455640; 463982; 469784; 459463; 460111; 450397; 459872; 462547; 467260; 462935; 453217; 455878; 462238; 471032; 471225; 457453; 470057; 473031; 458014; 452162; 466507; 458198; 470332;
452793; 459964; 456981; 465780; 466859; 468067; 454067; 451887; 450160; 462103; 453344; 460032; 450305; 473374; 450977; 468943; 457954; 465511; 460119; 470965; 451963

TABLE 3B
non-primate markers, tcdd; hl, ez, hg

dog subset 1 (tcdd)
479427; 612867; 487899; 475931; 480113; 483378; 607496; 609822; 609567; 486834; 610304; 476006; 474860; 479329; 477923; 403626; 403924; 485069; 610935; 480676; 403560; 488195; 487436; 480440;
479107; 483028; 478043; 478298; 607298; 607640; 484712; 487748
dog subset 2 (tcdd)
488473; 488472; 479329; 403651; 403824; 487649; 488922; 607007; 404306; 442955; 488508; 475032; 475705; 479074; 481732; 607827; 403612; 480438; 478854;
491618; 478151; 474663; 481124; 478781; 477886; 611999; 489432; 403885; 608445; 477535; 475450; 475312; 475083; 608196; 478868; 477166; 480011; 483266;
611484; 479483; 481756; 403606; 484335; 612199; 609421; 490904; 486758; 489532; 477746; 478347; 490863; 484648; 485692; 476915; 477221; 483729; 403999;
486407; 486507; 478471; 485864; 611193; 485056; 482140; 403463; 481628; 477039; 487486; 487731; 489620; 403968; 478918; 610636; 475085; 608060; 484056; 476336; 478902; 490532; 611171; 607083;
608147; 485103; 485915; 479747; 474922; 480381; 482604; 611694; 490860; 488471; 477052; 490201; 609622; 608850; 490926; 480557; 476146; 481457; 490402; 608864; 488257; 483728; 608486; 479032;
609035; 476952; 479409; 475323; 481988; 478507; 486762; 479760; 607575; 475465; 475718; 477199; 478533; 476693; 479723
dog subset 3 (tcdd)
489828; 478637; 477236; 478321; 403817; 442979; 403551; 485769; 475251; 490878; 483021; 477753; 483870; 476833; 479427; 442969; 476438; 403407; 486469;
442968; 403954; 482628; 487718; 483763; 481744; 403523; 449028; 403416; 403618; 488124; 403905; 486607; 403437; 488282; 482737; 403567; 486896; 488473;
487432; 488472; 484671; 610164; 485338; 484610; 486564; 607448; 611695; 486241; 403469; 610454; 479329; 488997; 480527; 442858; 480169; 489775; 479613;
481131; 474890; 403429; 481563; 403858; 485972; 477641; 403651; 403824; 403496; 479866; 403923; 487649; 477185; 488922; 477032; 483489; 479649; 476167;
494010; 483038; 474177; 415128; 479740; 477807; 610935; 488824; 607007; 486276; 485839; 611243; 611103; 403506; 403657; 404306; 481528; 487421; 610283;
442955; 477373; 403883; 488508; 493995; 403640; 403639; 607474; 488942; 403619; 487924; 403495; 403727; 490792; 475032; 483352; 478394; 480906; 486079;
475705; 490971; 486400; 403418; 612283; 478227; 478103; 483312; 480501; 478575; 479912; 611366; 477556; 476731; 606988; 479714; 490630; 478493; 480110;
479074; 478952; 403441; 480348; 481732; 607827; 474861; 474862; 481731; 474860; 611838; 442987; 485884; 474964; 478450; 610982; 403735; 477396; 403612;
480438; 478854; 480475; 403801; 483664; 484067; 491618; 479967; 479122; 403850; 483665; 486148; 489208; 403789; 488429; 486493; 489905; 489928; 488437;
477956; 403788; 483318; 609429; 477601; 478151; 478320; 403607; 481124; 478781; 479072; 477886; 612631; 480144; 610902; 476196; 480488; 611999; 609969;
489368; 403537; 403693; 480481; 476262; 403438; 610303; 442978; 477683; 477019; 479417; 403733; 403445; 489432; 403885; 403823; 403453; 494002; 491924;
478230; 448784; 609946; 483995; 477232; 482978; 611315; 608445; 479805; 477535; 478813; 442859; 474897; 475450; 475312; 480558; 480559; 606932; 403784;
483148; 610439; 481726; 475083; 608196; 474472; 474984; 609087; 488746; 403476; 607885; 481417; 477596; 477166; 478600; 477597; 480011; 483266; 480048;
403426; 485874; 403606; 490021; 484335; 612199; 609421; 479975; 490904; 484903; 479577; 486758; 489532; 477746; 474652; 478347; 488241; 479363; 403408;
403832; 442942; 475108; 483759; 403955; 490863; 484648; 479158; 485692; 476915; 477221; 478127; 479446; 483729; 610499; 485650; 486839; 403999; 403994;
490439; 474682; 488165; 490939; 486407; 476258; 486507; 488405; 479781; 478471; 403743; 485864; 403475; 488449; 403892; 485056; 486303; 482140; 403412;
488213; 403463; 607720; 403703; 403998; 481628; 477039; 487486; 487731; 476314; 403417; 403922; 484006; 403634; 487437; 477229; 403869; 489620; 612709;
480673; 490867; 480019; 478918; 610636; 478874; 475085; 488646; 612662; 608060; 403982; 474681; 403802; 482932; 484056; 477991; 476336; 493997; 476447;
490532; 611171; 489398; 607083; 476324; 485934; 608147; 483724; 485103; 611316; 479747; 609673; 612125; 474922; 480955; 476512; 480381; 609233; 491084; 611262; 482604; 477936; 611694; 612960;
490860; 488471; 477052; 490201; 490926; 476146; 612491; 479418; 403625; 482364; 486242; 481457; 490402; 481296; 609790; 487608; 479603; 608864; 488257; 483728; 482271; 491064; 479684; 490901;
608707; 476188; 484198; 608675; 608486; 486075; 608231; 481150; 479032; 609035; 476952; 475670; 479409; 478848; 475323; 488982; 612153; 609532; 474666; 476724; 478507; 486762; 487747; 479760;
607575; 475465; 478876; 477199; 478533; 481376; 476693; 608049; 484835
rat subset 1 (tcdd)
64547; 83584; 25406; 24296; 25315; 25283; 81663; 24401; 494499; 500661; 24426; 309622; 294270; 25599; 293963; 24539; 24577; 299626; 24314; 25496; 362282; 306886; 313689; 299339; 83472; 497811;
316626; 316737; 305795; 24385; 304078; 503160
rat subset 2 (tcdd)
64044; 25599; 29393; 84352; 315707; 114122; 25312; 25433; 24329; 313052; 59323; 54250; 25150; 29424; 294274; 294273; 294254; 299331; 497838; 24484; 313050;
497722; 299858; 81515; 60450; 81683; 25335; 81687; 25135; 25744; 25336; 25337; 81526; 300303; 301430; 25737; 24640; 298947; 315713; 363633; 25682; 25664;
117281; 65179; 56782; 24680; 50689; 114509; 170851; 362787; 24703; 25597; 24708; 309165; 25544; 361825; 24790; 25353; 83805; 117526; 24816; 84492; 301285;
29591; 81810; 304109; 362675; 25360; 316531; 314981; 296603; 54319; 25696; 363247; 24884; 24919; 313666; 64158; 54266; 84584; 288471; 501104; 29419; 314756;
266610; 114587; 364420; 117279; 301164; 113894; 361420; 360640; 360600; 311021; 60323; 303101; 78966; 500929; 192218; 246240; 293024; 500695; 290447; 116724;
367072; 299976; 161452; 500377; 304294; 311332; 313876; 311567; 266670; 300043; 288916; 304591
rat subset 3 (tcdd)
116631; 361925; 24165; 29650; 25026; 25645; 24177; 29146; 25690; 24185; 266802; 25290; 83589; 29722; 292697; 25728; 54226; 246097; 25385; 117273; 64186;
304127; 64639; 116502; 24887; 58919; 24224; 24888; 303836; 25181; 64625; 114496; 497672; 306956; 64314; 25402; 64026; 64044; 58918; 29161; 29224; 25729;
315291; 25109; 497761; 25073; 171369; 25406; 24251; 25599; 54237; 360621; 83502; 497718; 307618; 361432; 94201; 114851; 83571; 24252; 24253; 81645; 25590;
117258; 29393; 84352; 290905; 497663; 116630; 315707; 116549; 114122; 84353; 311163; 24291; 64001; 24296; 24297; 361523; 246070; 25086; 171352; 25146; 25112;
24314; 25312; 25195; 25433; 399489; 307794; 116651; 171361; 25313; 24329; 24330; 366492; 60465; 313052; 65030; 24337; 59323; 25149; 303828; 25439; 24360;
25598; 54250; 294515; 293890; 54251; 314322; 25319; 25150; 367562; 363735; 25172; 54254; 303539; 24392; 29739; 25686; 81666; 25721; 81670; 81504; 29143; 84027;
501110; 24424; 57298; 29438; 25260; 316123; 294236; 353256; 171528; 58940; 29424; 155012; 24446; 29560; 294274; 294273; 309621; 294269; 294270; 25460; 24451;
25735; 79256; 58917; 502373; 363266; 29577; 294254; 299331; 497838; 25462; 360647; 25712; 24483; 25151; 24484; 83476; 297767; 24506; 25467; 170921; 311144;
315346; 308995; 25021; 296456; 24511; 309684; 25724; 29147; 24516; 24525; 294853; 313050; 300438; 170905; 24538; 25330; 299858; 116565; 81515; 362391; 29357;
25631; 50554; 60450; 60661; 78967; 60430; 314856; 303604; 308106; 315119; 25332; 171341; 81683; 291234; 497834; 315606; 81686; 171045; 25335; 81687; 81707;
24565; 81709; 81521; 362657; 498982; 296344; 298894; 85482; 25135; 293453; 25744; 83619; 81736; 29508; 25336; 25337; 191575; 83782; 291084; 24600; 29492;
56761; 406162; 81526; 300303; 497735; 58813; 29613; 497733; 24617; 29431; 500242; 25737; 140544; 300257; 24640; 298947; 29692; 24653; 25619; 25738; 25664;
302934; 117281; 65179; 56782; 293508; 24680; 29355; 25522; 50689; 114509; 50658; 170851; 24683; 24684; 29192; 50557; 29527; 25614; 293694; 363875; 362787;
24703; 25676; 25597; 24708; 81758; 315655; 309165; 316164; 246074; 25544; 25651; 362264; 85385; 287280; 25351; 24779; 361825; 24787; 24790; 367846; 24796;
25353; 305614; 83805; 78968; 25124; 83783; 57027; 24816; 24817; 84492; 301965; 306862; 301285; 361178; 64678; 59086; 29591; 81810; 304109; 362533; 29260;
24835; 25625; 497808; 64550; 24842; 362675; 311786; 362788; 311245; 316531; 301442; 314981; 83586; 81817; 296603; 25361; 83529; 83785; 114111; 54319; 81818;
25696; 290805; 84495; 24884; 24919; 353227; 313666; 293960; 311764; 64158; 25445; 54266; 294326; 288471; 304323; 117274; 501104; 26295; 309295; 65137; 29419; 58945; 140668; 246756; 314756; 246775;
362491; 266610; 140673; 114587; 364420; 117279; 301164; 113894; 360640; 311021; 60323; 292732; 315608; 246253; 312331; 360801; 303101; 78966; 299694; 362989; 315756; 298652; 500929; 192218; 29374;
303477; 83726; 303634; 64537; 307366; 291005; 79111; 246240; 140657; 314870; 293024; 500695; 298604; 290447; 116724; 313977; 367072; 363228; 307927; 315173; 84010; 291582; 362850; 161452; 500377;
245975; 304294; 311332; 363237; 311567; 266670; 300043; 360992; 288916; 289608; 94172
mouse subset 1 (tcdd)
mouse subset 2 (tcdd)
12370; 16149; 12842; 12843; 12988; 13003; 13406; 109620; 15200; 13649; 269587; 13869; 14173; 14360; 231103; 15194; 14998; 14999; 193740; 15519; 15510; 16009;
16818; 16906; 16971; 545422; 17096; 17179; 545943; 17393; 17395; 17975; 18016; 18045; 18046; 18133; 434373; 18393; 18538; 18640; 30955; 18854; 20020; 19015;
19016; 51792; 19060; 19073; 18750; 26417; 23939; 26419; 26395; 26448; 110157; 114713; 19645; 19650; 19697; 20339; 20587; 20683; 20750; 20779; 21374; 21390;
21682; 21419; 21812; 21813; 21825; 21844; 22062; 22095; 22145; 434428; 12818; 22325; 22350; 22359; 22596; 22612; 22632; 22642; 22142; 17123; 17979; 100683;
109115; 18222; 12905; 14082; 13386; 21933; 12633; 18519; 18412; 51886; 21341; 21340; 21339; 53859; 70834; 64685; 16801; 103806; 213498; 22143; 214158; 81601;
56532; 66354; 105689; 69632; 382985; 16842; 53857; 108086; 54485; 50754; 12211; 16396; 67014; 102060; 74198
mouse subset 3 (tcdd)
17960; 11423; 56456; 11486; 11487; 11535; 11556; 11576; 16449; 11622; 11651; 11682; 11689; 30948; 319924; 11785; 11806; 11813; 11816; 11820; 14102; 14103;
11848; 17172; 12013; 12015; 12018; 12028; 12443; 12043; 12048; 12053; 12111; 12122; 12124; 12189; 12231; 12366; 12367; 12369; 12370; 12371; 12391; 12408;
268697; 12447; 12455; 12480; 12481; 12527; 20778; 21939; 12505; 12508; 16149; 12534; 23834; 12550; 12558; 12562; 12555; 12567; 12575; 12576; 12606; 12608;
243764; 11438; 12805; 12842; 12843; 12826; 12914; 12981; 12988; 12995; 13003; 12387; 12388; 66473; 109672; 13076; 13077; 13078; 13088; 13095; 13106; 13113;
13074; 13197; 18104; 13406; 13492; 15200; 14357; 13555; 104394; 13559; 13627; 13645; 13649; 13653; 13836; 13043; 269587; 13850; 13866; 13869; 13871; 13982;
13983; 104156; 14062; 14080; 14079; 14173; 14198; 56484; 77836; 14254; 14281; 14319; 14360; 14387; 14390; 14460; 14463; 231103; 14534; 14598; 14609; 14630;
14677; 14682; 14719; 76282; 14784; 14824; 56637; 14863; 14864; 14871; 98053; 14885; 14886; 14939; 51788; 15078; 15194; 12628; 15234; 15251; 14998; 14999; 14960;
14968; 14969; 15366; 15368; 15378; 15381; 15458; 15461; 15463; 15205; 193740; 15519; 15510; 15528; 15896; 15978; 16002; 16004; 16009; 16007; 19664; 16334;
16367; 16398; 16399; 16401; 16402; 16408; 16409; 16410; 16412; 16414; 16416; 192897; 16450; 16476; 16653; 16668; 16818; 16835; 16848; 15450; 16890; 16971;
545422; 16976; 17096; 17119; 17126; 17127; 17128; 17179; 17187; 84004; 17210; 17246; 26406; 26407; 17295; 17305; 17314; 56615; 545943; 17345; 17350; 214162;
17390; 17392; 17393; 17395; 17387; 17250; 17685; 17698; 17769; 17829; 17859; 17863; 17865; 18109; 27354; 17975; 70316; 18016; 18024; 18033; 18044; 18045; 18046;
18102; 18103; 18105; 18127; 18129; 18131; 18132; 18133; 434373; 22608; 23959; 18213; 18550; 18787; 18479; 23983; 18521; 18538; 13026; 56612; 18640; 30955;
18783; 18792; 18803; 19016; 19041; 51792; 19060; 19073; 18749; 18750; 320795; 18762; 26417; 23939; 26419; 26420; 26395; 19109; 19116; 19164; 19211; 19225;
14083; 26412; 19353; 26448; 110157; 218397; 114713; 19645; 19650; 19651; 19684; 19697; 58988; 20230; 20249; 20339; 20344; 67996; 20416; 21402; 20526; 20533;
20587; 20656; 20683; 20687; 20737; 20750; 20742; 20779; 20787; 20846; 20887; 11491; 21390; 21405; 21682; 21752; 21418; 21419; 21781; 22042; 21803; 21812; 21813;
21825; 21844; 21885; 21898; 21926; 21929; 21937; 21938; 21969; 22059; 22062; 22029; 22030; 22031; 22033; 22034; 22145; 434428; 22218; 12818; 22278; 22282; 22325; 22329; 22333; 22339; 22341; 22350; 22352;
22359; 22427; 22594; 74335; 22612; 22632; 235320; 22642; 22682; 228913; 22142; 14283; 17123; 28295; 100683; 17120; 23957; 109115; 108155; 16151; 56505; 18222; 56455; 76408; 71609; 12905; 22035; 192656;
14082; 108124; 13386; 21933; 12633; 18519; 18412; 51886; 53859; 64685; 16801; 18223; 140630; 11450; 18025; 20602; 103806; 213498; 77976; 67057; 63958; 22143; 214158; 81601; 12464; 140486; 13018; 14674;
15442; 240354; 23882; 26459; 56532; 244486; 14084; 73914; 66354; 56381; 105689; 14245; 69632; 56363; 102162; 170770; 223722; 13388; 225579; 57875; 16842; 53857; 224440; 108086; 54485; 50754; 57750;
16396; 67014; 71701; 102060; 326619; 67980; 26457
dog subset 1 (hl)
477023; 607852; 481088; 610683; 442987; 478889; 476882; 474614; 491903; 612603; 403492; 479344; 478019; 477858; 490735; 476588; 476467; 489690; 483792; 475937; 403892; 480129; 483793;
477938; 475491; 486839
dog subset 2 (hl)
475582; 477498; 609233; 486638; 475089
dog subset 3 (hl)
481651; 483322; 478757; 477236; 490878; 474808; 476600; 607705; 487718; 475867; 403474; 474890; 484790; 607922; 478806; 403856; 476167; 489664; 415128;
480472; 480113; 480516; 476063; 475582; 477295; 475566; 489375; 403755; 475705; 488231; 607942; 611942; 476825; 480348; 479864; 485884; 478450; 442945;
403735; 483146; 607570; 474630; 403612; 403850; 442990; 492244; 477675; 403616; 483793; 476062; 482945; 611940; 478813; 477498; 478987; 491866; 487139;
487797; 403849; 403606; 477575; 486758; 610411; 479564; 403559; 486507; 488405; 403892; 484926; 403412; 403998; 489932; 485867; 403922; 403802; 483791;
485384; 483792; 485591; 609126; 607809; 486638; 612251
rat subset 1 (hl)
rat subset 2 (hl)
58945; 25560; 171159;
rat subset 3 (hl)
497803; 50681; 79558; 24165; 24185; 25374; 29757; 304127; 116599; 24248; 114851; 60463; 81646; 81649; 64001; 79129; 24296; 25086; 287765; 25315; 303547; 84490;
54250; 84482; 25318; 29573; 498881; 117533; 501069; 60666; 292804; 24440; 29560; 79239; 25735; 79256; 24465; 502373; 360348; 25322; 79244; 294254; 89784;
116553; 50687; 24534; 25608; 84575; 286922; 81829; 337868; 83619; 360265; 302552; 117276; 25741; 65152; 25664; 116590; 50689; 114100; 25541; 24786; 24790; 367846; 83783; 29338; 301965; 59086; 84574;
56083; 24835; 83785; 293735; 361627; 83512; 301062; 316064; 25560; 310645; 24861
mouse subset 1 (hl)
113868; 104112; 14381; 432452; 15368; 15926; 16828; 17449; 18639; 17117; 14377; 16548; 208715; 56421; 18242; 110606; 21881; 51798; 15486; 56473; 21351; 20887;
110196; 76267; 67460; 66234; 52538; 20249; 13119
mouse subset 2 (hl)
14182; 18125; 56455; 20928; 22770
mouse subset 3 (hl)
11303; 11430; 11477; 11486; 11651; 17025; 11655; 11975; 12013; 109778; 12359; 12575; 12772; 12912; 11909; 26416; 109672; 13057; 13076; 13106; 13207; 13849;
14055; 14156; 14173; 14182; 56458; 14272; 14385; 433273; 231103; 66355; 54214; 14555; 14751; 15130; 15251; 15369; 15378; 15452; 15461; 15492; 15485; 15488;
193740; 319554; 16163; 16728; 16832; 16846; 76267; 60527; 17448; 17927; 18024; 18125; 18214; 209815; 170768; 18641; 18642; 19016; 26413; 26417; 235293; 20280;
545845; 20683; 20687; 20887; 21672; 21752; 21803; 21804; 21817; 21926; 22339; 24115; 20841; 56473; 208194; 102448; 108737; 20928; 68603
dog subset 1 (ez)
476323; 610935; 481500; 403800; 403523; 474867; 474865; 474479; 486281; 488449; 474905; 609822; 480113; 442978; 479303; 479441; 479329; 403904; 476100;
476450; 474841; 484822; 488559; 491061; 403400; 479744; 403435; 449028; 607745; 486100; 477858; 403670; 488784; 479171; 481007; 477991; 612291; 482861;
403629; 474890; 403816; 475596; 483006; 478432; 481731; 474860; 480360; 403693; 478584
dog subset 2 (ez)
403945; 611541; 403416; 488770; 403618; 484472; 486607; 403437; 480416; 481131; 475537; 609920; 474890; 403429; 481563; 611216; 477796; 487151; 607922;
479866; 487786; 479441; 474403; 477185; 474844; 477032; 486077; 480472; 404306; 476105; 403640; 478293; 477295; 481084; 403845; 487351; 483312; 479960;
485828; 487309; 480348; 481732; 607827; 474860; 474860; 403612; 403979; 476412; 478666; 404021; 489800; 476196; 476160; 403537; 403693; 478939; 609832;
415126; 491946; 448784; 477232; 403924; 485069; 442859; 477166; 403781; 484431; 481772; 487235; 484963; 482840; 485874; 403602; 611484; 482686; 479483;
483863; 480533; 491303; 403556; 480338; 489749; 481014; 476915; 477221; 478127; 483729; 479112; 479949; 475080; 403787; 480143; 475851; 483954; 608403;
489066; 490439; 476710; 486507; 485864; 488449; 481111; 490967; 484196; 491950; 481740; 611193; 403998; 487486; 403601; 483343; 403922; 403634; 487437;
403869; 489620; 485406; 487509; 480896; 478874; 475085; 474681; 486588; 474609; 611171; 475864; 607083; 479854; 485915; 483121; 479955; 477392; 478350; 475684; 480381; 609233; 611262; 482048; 477052;
477302; 611249; 480147; 476146; 476158; 490263; 489162; 477664; 474822; 610191; 484907; 483999; 474494; 475217; 481396; 478907; 474362; 607899; 480818; 609018; 483060; 483728; 476018; 484198; 610407;
607060; 484028; 607896; 608367; 477541; 477615; 480709; 607947; 609080; 612153; 475587; 482895; 491221; 476579; 612505; 478474; 477639; 609794; 475258; 480158; 487128; 477864; 480668; 480949; 481104;
481958; 475913; 403518; 482787
dog subset 3 (ez)
477699; 491292; 489828; 403580; 608134; 478321; 490385; 403962; 475251; 607538; 490878; 610941; 489526; 483021; 489458; 475630; 480830; 476322; 476094;
479325; 479139; 482558; 442936; 403945; 403407; 486469; 442968; 403588; 475176; 403954; 403670; 483218; 485217; 475924; 611541; 485189; 607262; 474457;
612911; 488513; 479450; 403669; 479901; 477595; 478807; 478393; 487981; 477101; 484490; 478284; 487718; 483763; 481744; 403523; 449028; 403416; 488770;
403618; 612349; 607482; 485756; 403762; 403905; 484472; 486607; 486080; 486127; 490695; 491750; 608552; 403437; 482682; 478194; 442974; 480416; 476694;
610764; 489050; 483745; 479148; 482737; 403567; 487899; 486896; 488473; 487432; 403474; 487746; 487366; 485187; 483845; 485338; 475007; 611782; 608847;
484610; 479303; 478442; 403931; 403646; 403469; 403468; 474391; 479329; 488997; 442858; 489775; 481131; 475537; 609920; 491325; 474890; 403429; 481563;
475359; 611216; 609346; 486028; 610125; 609010; 492302; 403858; 477796; 442971; 484790; 448802; 403541; 484789; 403651; 403824; 403826; 487151; 607922;
478806; 479866; 487786; 479441; 607506; 474403; 403856; 477257; 611795; 477185; 474844; 488922; 479991; 477032; 479649; 486077; 483662; 479065; 608843;
476167; 494010; 483038; 474177; 415128; 479740; 477807; 474873; 483106; 607439; 480472; 478924; 489573; 609048; 474479; 610935; 489462; 478767; 479935;
607007; 485839; 611243; 403657; 404306; 481528; 487870; 481565; 480466; 610283; 477373; 477686; 403883; 481105; 488508; 612282; 493995; 403640; 403639;
489287; 483324; 488942; 403619; 607277; 478293; 403462; 478984; 476063; 475582; 488808; 489095; 481084; 403845; 490792; 478232; 478394; 403755; 486079;
609393; 480495; 486400; 403570; 403873; 612283; 481126; 488448; 487351; 491952; 442961; 483312; 478047; 478279; 478575; 479912; 611366; 477556; 475597;
479714; 485828; 487309; 475035; 487551; 608175; 480348; 474667; 474836; 474838; 481732; 607827; 607786; 474861; 474862; 481731; 474860; 474860; 609564;
483594; 442946; 442987; 480979; 480100; 608958; 607052; 403735; 476411; 475124; 489766; 403612; 403979; 611362; 480438; 609146; 489673; 403801; 476216;
610255; 442951; 491618; 608559; 482839; 403785; 403790; 403985; 403850; 403628; 610823; 403977; 403976; 442990; 403796; 478381; 483665; 485209; 475554;
492247; 481500; 475633; 476412; 482264; 489208; 477956; 442952; 484185; 610004; 609429; 480522; 482154; 403811; 478666; 478585; 444857; 486513; 477601;
478151; 403616; 404021; 479193; 489800; 486363; 481124; 607183; 481712; 482599; 477886; 610286; 476070; 610902; 488504; 480218; 476160; 403537; 481839;
610519; 403693; 478939; 476262; 483758; 403438; 487416; 442978; 477683; 490008; 477019; 479417; 489428; 403733; 403445; 489432; 489429; 403885; 486408;
403823; 403453; 494002; 491924; 403768; 804478; 478230; 403745; 477232; 403924; 482978; 485069; 613013; 611315; 607700; 488220; 488431; 478813; 442859;
486858; 481711; 474897; 479051; 480559; 606932; 403822; 482260; 484147; 491866; 611921; 488746; 607885; 612814; 481417; 477166; 479875; 610775; 476371;
403621; 403879; 484431; 481772; 487235; 484963; 482840; 476446; 485874; 482083; 403602; 489971; 611484; 490360; 403606; 490021; 478053; 491303; 612199;
479351; 403556; 490904; 494005; 609934; 490722; 484903; 474176; 477575; 486758; 478311; 477746; 607019; 611397; 488241; 485783; 480338; 490123; 475040;
480246; 474865; 474867; 489749; 477467; 481014; 480256; 480258; 403832; 442942; 475108; 477263; 490563; 486717; 477488; 403955; 474539; 483225; 477045;
486508; 476915; 477221; 478127; 483729; 479112; 489798; 477308; 478667; 477499; 475454; 480181; 479949; 475080; 403787; 480143; 475851; 485028; 403676;
480865; 483954; 486839; 448788; 403981; 480600; 448786; 403522; 491148; 448792; 403586; 490356; 487265; 449622; 403999; 403994; 608403; 612817; 489066;
490439; 489536; 491606; 476710; 486407; 485334; 478478; 485837; 403559; 476258; 486507; 488405; 611255; 478471; 488616; 403484; 485864; 612861; 403993;
481272; 488449; 481111; 490967; 478845; 490968; 490969; 403892; 484196; 491950; 474864; 481740; 485079; 475448; 403412; 403998; 477039; 487486; 403601;
403816; 403633; 481289; 483343; 448807; 403417; 403922; 491161; 403634; 487437; 480525; 403869; 490397; 489620; 404019; 485406; 481895; 609156; 474536;
487509; 480896; 612742; 478874; 475085; 442965; 403982; 481590; 474681; 479255; 486588; 403802; 482932; 494000; 399544; 491867; 476847; 486560; 493997;
474609; 476447; 490532; 611171; 475864; 607083; 483900; 479854; 481713; 483724; 485915; 479747; 490099; 486271; 609673; 607683; 477392; 475451; 480955;
484665; 478350; 478210; 488125; 475684; 480381; 490006; 491084; 611262; 482048; 477052; 477302; 442949; 480147; 476146; 487057; 484802; 490831; 490263;
489162; 403683; 482101; 481241; 484907; 610825; 487158; 476708; 482715; 482364; 483999; 487524; 487439; 474494; 475217; 612867; 476818; 476540; 477474;
403702; 481396; 478907; 403689; 474362; 484309; 607899; 480818; 480190; 479465; 609018; 483060; 483728; 480328; 481233; 484198; 477239; 607060; 481267; 484955; 480287; 607929; 486338; 475490; 609600;
477186; 481809; 490874; 474579; 481262; 607896; 608367; 477541; 480709; 611920; 609743; 477207; 606908; 480595; 607947; 609080; 612153; 477818; 489925; 482895; 491221; 476579; 478974; 476659; 612505;
478507; 478474; 477639; 485049; 609794; 403502; 475258; 477526; 479760; 489412; 482745; 610883; 475385; 482744; 477864; 491306; 480949; 481104; 481414; 481958; 475330; 475913; 478128; 609866; 610315;
612601; 403518; 607671; 482787; 486723; 608568; 607880; 488237
rat subset 1 (ez)
rat subset 2 (ez)
287774; 360500; 24224; 170929; 24888; 81613; 64625; 497672; 24242; 94201; 140908; 114483; 114851; 83571; 309361; 58954; 81646; 497663; 25420; 170587; 116549;
84353; 64529; 25112; 287765; 24329; 25043; 170915; 291703; 83727; 84482; 294515; 293860; 25661; 81659; 313017; 81504; 81673; 25734; 297893; 29560; 294274;
294273; 294270; 294270; 294254; 24471; 360415; 292892; 83628; 288001; 83781; 65172; 50554; 60430; 314856; 304798; 81683; 296344; 24577; 299626; 81736; 25737;
363272; 25344; 24649; 298696; 29692; 25692; 25738; 85253; 315713; 289758; 363633; 364834; 497991; 362102; 25636; 29670; 291983; 24708; 81758; 309165; 89809;
81763; 24615; 85247; 81778; 498209; 307947; 85385; 171379; 24790; 83805; 25124; 288774; 25125; 25155; 317256; 25217; 117526; 59086; 81812; 24835; 25625;
497808; 24842; 362675; 292925; 25232; 25573; 301442; 314981; 83529; 24873; 85252; 24919; 25578; 313666; 360471; 84584; 312492; 360908; 301555; 300772; 313929;
29419; 58945; 246756; 65154; 301164; 295052; 85383; 83422; 311021; 291414; 363603; 312849; 309565; 84591; 288905; 293017; 300084; 114485; 114552; 289820;
301511; 116633; 305896; 83427; 25514; 65037; 192218; 293674; 291005; 192269; 25577; 308267; 362201; 300656; 499882; 305475; 296639; 81511; 500870; 364624;
306492; 360564; 290541; 292156; 85311; 366975; 297695; 266688; 25309; 307545; 307126; 361666; 296562; 291279; 304606; 294520; 287830; 308516; 295620; 500110
rat subset 3 (ez)
24153; 116631; 311860; 81822; 303742; 29650; 25591; 24173; 25690; 25373; 24185; 25375; 266802; 81639; 85333; 498885; 25380; 25673; 79125; 24205; 79116; 287774;
54226; 246097; 25385; 24208; 29183; 117273; 64373; 295342; 308875; 360678; 360500; 25387; 25388; 315222; 25389; 81643; 300711; 171374; 171375; 171082; 114630;
94271; 360716; 83625; 24223; 304127; 64639; 116502; 24887; 58919; 24224; 170929; 24888; 292700; 309696; 25394; 25295; 25181; 81613; 64625; 114496; 364403;
83470; 25296; 25644; 367786; 140923; 497672; 64022; 29618; 298566; 24242; 64202; 24246; 171140; 29153; 25403; 25166; 64314; 25402; 83584; 64026; 64044; 58918;
24248; 367218; 50662; 156726; 29345; 114494; 114839; 64033; 25729; 25405; 29157; 25109; 24932; 25660; 361584; 171369; 84349; 25406; 29186; 25599; 54237; 83502;
307618; 94201; 140908; 114483; 362110; 114851; 83571; 246060; 25164; 54238; 289993; 66019; 24252; 24253; 499712; 29271; 24255; 81645; 309361; 24854; 60463;
117027; 171054; 117029; 29393; 84352; 25412; 58954; 81646; 497663; 25419; 25420; 24277; 170587; 81649; 362273; 78965; 116549; 114122; 29317; 25307; 366219;
24962; 307505; 84353; 24291; 64529; 171293; 25425; 29175; 64001; 24296; 24297; 361523; 246070; 25086; 171352; 25146; 140926; 29167; 25112; 29467; 287765;
363256; 298298; 24312; 25035; 24314; 497670; 25253; 25433; 399489; 307794; 315219; 25313; 24329; 24330; 117045; 497806; 363854; 25043; 60465; 170915; 65030;
312812; 24337; 29496; 59323; 292673; 25149; 24356; 303687; 29251; 25439; 24360; 24361; 83727; 171075; 295279; 289211; 304966; 84490; 54250; 84489; 25114;
293860; 25661; 314322; 56718; 25320; 363735; 81659; 498881; 54254; 29244; 24387; 394266; 316033; 29739; 140589; 24398; 313017; 84475; 24404; 29328; 81504;
24413; 29468; 84027; 24422; 24424; 29438; 25260; 306516; 353256; 25734; 297893; 84577; 24443; 79237; 29560; 500155; 24974; 414790; 294274; 294273; 365542;
24986; 309621; 294269; 294270; 294270; 294228; 309600; 24973; 117062; 24451; 117282; 117280; 287647; 368178; 502373; 60421; 291960; 294254; 24471; 161476;
299331; 24477; 292060; 360415; 298205; 25712; 116465; 24482; 25718; 24484; 289560; 84351; 287287; 24497; 24498; 25325; 117539; 84405; 64546; 116553; 29197;
306626; 24506; 65038; 64194; 363520; 24508; 290749; 292892; 296953; 293624; 170921; 24511; 84598; 24514; 25326; 24516; 81679; 83628; 64030; 288001; 288064;
24525; 300250; 294853; 313050; 25608; 83781; 116641; 65172; 29524; 299858; 25008; 361795; 311337; 81682; 81515; 494343; 297176; 25631; 25595; 291439; 60430;
316273; 291885; 81517; 314856; 304798; 308106; 29417; 313662; 25332; 171341; 85483; 81683; 246759; 497834; 315606; 300339; 81686; 171045; 25335; 63849; 81687;
117061; 25481; 81707; 24565; 81709; 81521; 26195; 362657; 286918; 296344; 24577; 298894; 299626; 288770; 85482; 114553; 291044; 29458; 83619; 81736; 309452;
361794; 29508; 499194; 83782; 291084; 24599; 312195; 25054; 113929; 302552; 289747; 497733; 29431; 29432; 500242; 25737; 81745; 308321; 25154; 25344; 24649;
298696; 29692; 25692; 25738; 64204; 85253; 25515; 315713; 171068; 25664; 302934; 60660; 362102; 65179; 65248; 25636; 24680; 170538; 29340; 81749; 29355;
360748; 116590; 50689; 24683; 24686; 314615; 29670; 29672; 94198; 29425; 85492; 24968; 24967; 291983; 161475; 29630; 29614; 50557; 29527; 25614; 24697; 117063;
116689; 25622; 363875; 294429; 295347; 24706; 24708; 81758; 309165; 89809; 116468; 288414; 288003; 304528; 361390; 81762; 81763; 25347; 24615; 85247; 81778;
64196; 362890; 302642; 498209; 246074; 287562; 116637; 81780; 287561; 29397; 117518; 298006; 117551; 305236; 171371; 89808; 24772; 25544; 25651; 307947;
494445; 85385; 287379; 29723; 84386; 171379; 361825; 308909; 29219; 362242; 24786; 24787; 24790; 367846; 366126; 25353; 83805; 501099; 24797; 497823; 25124;
288774; 25125; 367264; 24918; 25126; 362896; 83783; 25155; 317256; 24811; 24812; 25217; 171046; 114521; 301965; 24827; 59086; 81810; 81812; 116510; 29543;
25358; 310553; 29260; 24835; 287543; 25625; 497808; 24842; 362675; 362862; 292925; 315852; 85489; 116484; 58819; 25232; 81920; 25573; 301442; 83808; 314981;
289801; 25600; 25361; 116643; 83529; 83531; 24873; 83785; 114111; 24874; 116669; 317371; 308937; 24881; 290805; 289754; 85252; 84495; 24884; 24919; 25578;
313666; 60628; 360471; 294250; 25445; 84017; 84584; 288471; 79257; 304323; 361181; 301555; 307766; 26295; 300772; 171445; 246334; 313929; 29419; 252971;
140668; 246756; 246775; 364420; 65154; 301164; 295052; 89829; 83422; 311021; 362485; 498609; 314384; 363603; 29282; 83504; 300057; 366962; 288905; 432361; 140665; 116598; 312331; 293017; 64476;
300084; 114485; 114552; 64547; 84388; 297518; 288669; 81732; 289820; 301511; 170842; 116698; 116633; 305896; 83427; 301701; 363014; 25514; 65037; 192218; 116679; 315159; 291005; 311621; 25577;
315121; 114562; 314126; 363140; 114212; 290692; 293897; 311546; 316228; 314465; 85428; 362201; 300656; 499882; 305475; 296639; 293668; 295062; 362247; 66017; 114512; 117089; 81511; 500870; 64031;
282817; 306492; 360564; 290541; 116479; 314759; 292156; 161452; 85311; 366975; 362827; 297695; 25309; 304580; 304294; 24861; 315650; 114246; 291135; 500533; 116469; 361666; 291279; 304606; 312516;
294520; 313588; 287830; 307727; 316561; 85251; 362393; 308516; 295620; 500110; 171018; 365368; 364681
mouse subset 1 (ez)
11668; 13076; 18104; 16362; 17748; 18669; 21354; 12028; 16912; 16913; 109754; 231691; 20846; 14711; 14629; 13849; 17314; 12450; 12955; 16149; 13179; 22171;
20085; 11629; 23886; 12227; 11951; 13040; 11867; 12306; 12443; 14456; 268756; 18242; 11852; 17713; 331535; 22352; 57294; 211323; 394431; 12575; 21857; 14131;
14782; 234671; 216456; 68567; 15945; 14968; 14969; 73834; 14961; 17246; 70186
mouse subset 2 (ez)
11818; 14570; 12043; 12047; 12048; 12122; 12189; 12313; 12567; 12568; 12571; 12575; 12576; 12581; 12675; 23849; 12912; 12914; 12954; 12955; 12974; 12995; 13001;
12387; 13030; 13197; 13207; 13649; 13717; 328572; 13872; 13982; 14118; 56458; 56484; 192176; 14268; 231580; 55948; 14784; 229906; 15162; 433759; 15251; 14998;
14999; 14969; 14969; 193740; 15507; 52668; 54131; 12521; 16644; 16854; 16885; 17128; 17210; 17246; 17248; 545943; 17342; 54601; 17829; 17865; 17869; 17873;
18033; 18538; 18582; 18673; 18712; 18708; 23988; 18791; 18803; 18815; 18854; 18972; 20020; 69241; 19014; 110854; 18747; 19167; 19171; 53380; 19645; 19650;
19651; 19697; 19687; 19983; 20198; 20200; 20194; 394252; 20248; 56086; 20390; 20416; 20586; 20683; 20779; 20846; 20847; 20848; 20963; 270627; 21356; 21374;
21803; 21825; 21833; 21877; 21926; 21937; 21938; 22059; 22062; 22088; 22174; 22201; 22196; 22218; 12818; 22333; 22337; 103573; 22632; 22631; 22642; 252870;
17979; 26903; 12545; 26554; 56469; 17977; 18222; 56455; 71609; 22402; 18519; 12427; 78688; 17344; 64685; 67655; 17164; 69125; 108098; 70208; 18693; 50931;
72567; 56438; 23857; 12049; 66713; 76709; 56399; 16391; 14694; 18777; 17289; 81601; 66556; 23882; 20024; 22630; 68652; 17151; 58521; 74309; 54614; 68379; 16797;
105837; 170770; 67207; 18983; 76281; 71704; 54673; 85031; 223828; 60321; 235442; 13063; 58523; 70024; 66165; 18146; 64113; 18744; 22781; 171580; 445007; 52428;
326619; 76654; 68910
mouse subset 3 (ez)
232345; 17960; 11350; 11423; 11461; 71885; 11487; 11545; 11548; 11622; 11625; 11651; 11669; 11670; 11682; 11687; 11739; 433923; 16952; 11747; 11749; 11789;
11792; 11799; 11818; 11820; 14102; 14103; 11835; 11839; 11848; 11852; 11853; 56212; 192662; 14570; 109689; 216869; 11883; 11910; 108147; 11920; 11947; 11950;
67942; 228033; 11957; 11964; 26362; 12010; 12013; 12015; 12018; 12028; 12443; 12043; 12047; 12048; 12051; 110279; 12075; 12097; 12111; 12122; 12124; 12143;
12153; 12159; 12161; 12169; 12177; 12189; 12223; 12226; 12259; 12313; 12317; 108058; 12333; 12380; 12362; 12366; 12367; 12368; 12369; 12370; 12371; 12359;
12393; 12406; 12428; 268697; 51813; 12444; 12445; 12447; 12450; 12452; 12480; 12504; 12487; 233274; 21939; 21947; 12505; 12512; 16149; 12534; 216150; 12550;
12562; 12567; 12568; 12571; 107951; 12575; 12576; 12577; 12580; 12581; 12591; 12606; 12608; 229841; 12631; 12638; 243764; 12675; 12759; 12772; 12771; 12773;
12774; 12842; 12843; 12824; 23849; 12912; 11909; 12914; 12944; 12954; 12955; 12961; 12966; 12970; 12974; 26416; 110750; 12977; 12995; 13001; 13003; 13008;
13010; 58214; 107869; 12385; 12387; 66473; 13030; 13033; 13036; 13038; 109672; 13076; 13077; 13078; 13088; 13095; 13106; 13113; 13074; 13163; 13191; 13197;
13198; 13207; 11790; 74754; 13361; 109754; 18104; 13385; 13482; 99586; 109620; 15200; 13555; 104394; 72962; 13645; 13649; 13653; 13684; 15569; 15568; 13717;
13043; 328572; 13850; 13860; 13866; 13867; 13869; 13870; 13871; 13982; 13983; 23871; 14027; 14061; 14062; 14080; 14088; 14118; 14128; 14129; 14130; 246256;
14156; 14173; 14182; 14184; 14186; 192176; 14268; 14281; 56717; 14459; 14390; 231580; 433273; 14463; 14528; 14580; 14598; 14619; 12091; 14630; 14632; 14660;
55948; 12766; 14775; 14779; 14784; 14815; 14827; 56637; 14863; 14871; 68153; 14886; 15162; 433759; 15182; 15186; 15242; 15251; 14963; 110557; 14998; 14999;
15002; 14960; 14968; 14969; 14969; 217082; 15040; 15013; 15007; 15361; 15368; 15387; 51810; 15417; 15426; 15461; 15469; 15499; 26386; 193740; 15507; 69253;
15519; 15891; 15900; 52668; 15974; 15978; 15979; 16000; 16001; 16009; 29817; 16150; 16189; 16191; 16193; 16196; 16153; 16154; 16159; 16160; 16163; 16173; 26356;
16334; 16332; 231070; 16179; 16362; 16363; 54131; 27056; 54123; 16398; 16412; 16451; 16452; 16453; 16476; 16480; 12521; 16542; 16590; 16644; 16646; 16653;
16678; 16691; 16668; 16818; 16846; 16854; 56048; 16885; 16886; 16971; 16992; 16994; 17005; 17022; 17096; 56753; 56150; 17127; 17756; 17190; 17210; 17215; 17218;
17242; 17246; 17248; 26407; 17283; 17295; 17150; 17314; 56615; 12265; 545943; 17329; 17350; 214162; 83995; 17390; 17392; 17393; 17394; 17395; 17384; 17385;
17387; 17250; 17685; 17698; 17750; 17708; 17769; 17858; 17865; 17869; 18109; 17873; 17938; 27354; 17969; 18003; 18012; 18024; 18033; 18034; 18038; 18044; 68039;
18103; 18105; 18126; 18181; 18212; 19069; 209815; 18413; 18550; 18479; 224105; 23983; 18538; 18607; 18613; 18616; 18667; 18671; 18673; 18712; 18708; 23988;
18791; 18793; 18803; 18810; 18815; 18817; 18854; 18986; 19016; 19041; 72930; 110854; 19060; 105787; 18747; 18750; 18753; 18754; 18755; 320795; 19090; 26413;
26417; 50772; 26419; 19094; 19106; 19109; 19122; 19152; 19167; 26442; 19170; 19173; 19177; 16913; 16912; 19171; 67151; 53380; 19186; 19188; 19211; 19225; 14083;
19246; 19255; 15170; 19247; 19353; 19386; 109905; 218772; 19411; 19645; 19650; 19651; 19697; 19687; 69263; 106344; 72151; 24015; 19877; 19983; 67248; 20198;
20200; 20194; 224903; 67125; 20229; 394252; 20248; 20249; 20293; 20303; 20304; 20306; 20292; 20295; 20298; 20299; 56066; 16963; 20312; 20315; 20339; 20344;
56086; 20382; 20390; 20416; 20509; 20568; 20586; 20587; 20597; 20617; 20648; 545845; 20656; 20683; 20687; 20375; 20750; 20739; 20779; 20807; 20604; 20607;
20846; 20847; 20848; 20849; 20850; 20851; 20852; 20887; 20963; 270627; 21354; 21355; 21356; 21423; 21752; 21802; 21803; 21813; 21825; 21833; 21857; 21858;
21859; 110595; 21877; 24088; 21898; 21926; 21927; 21937; 21938; 21973; 22059; 209456; 22062; 22027; 22088; 22137; 22138; 22160; 22166; 50493; 22174; 22201;
56550; 66105; 22196; 22218; 231396; 12818; 22271; 22288; 22329; 269523; 22333; 22334; 22337; 22339; 22341; 22346; 22371; 22376; 22390; 22408; 22427; 22433;
103573; 22594; 74335; 22612; 22632; 22631; 22642; 12767; 252870; 53761; 14283; 15364; 17979; 100683; 12005; 72399; 17120; 99375; 26554; 93762; 108155; 52163;
56469; 110198; 22061; 17977; 18222; 12703; 76408; 71609; 22035; 21933; 22402; 18519; 12427; 12702; 17344; 64685; 12448; 53978; 73086; 17164; 69125; 65019; 16591; 79456; 17444; 50931; 19340; 12304;
18025; 72567; 329504; 170731; 56438; 23857; 12049; 12125; 16068; 68089; 56378; 74117; 66713; 76709; 22057; 21849; 56399; 16391; 14694; 67052; 22601; 100689; 18777; 17289; 81601; 68755; 57259;
23882; 67145; 22630; 104445; 12539; 217578; 56289; 50883; 77113; 13380; 72119; 21981; 104215; 26912; 17151; 58521; 74309; 68379; 107392; 97064; 56407; 80857; 56321; 12070; 16797; 105837; 170770;
18569; 66824; 18983; 76281; 71704; 16456; 67184; 54673; 16842; 85031; 223828; 59004; 60321; 81897; 13063; 231637; 108086; 56057; 64177; 66834; 66233; 194352; 66165; 227700; 64113; 18744; 22781; 23885;
171580; 76793; 445007; 109151; 77629; 12822; 94245; 69538; 52428; 326619; 76654; 68910; 71138; 246779; 218215
dog subset 1 (hg)
dog subset 2 (hg)
403835; 474819; 403831; 481689; 491618; 403870; 403851; 612271; 403545; 478906; 478905; 488429; 486493; 489905; 489928; 488368; 477598; 483345; 475720;
611884; 484066; 491431; 481826; 490497; 403885; 491924; 482582; 490344; 611940; 442859; 475083; 612021; 606755; 479483; 489816; 488561; 403987; 477488;
483729; 448786; 611927; 482986; 477945; 449622; 475471; 403475; 475239; 403949; 485079; 481519; 485867; 487486; 484087; 481289; 485698; 483844; 486004;
492072; 489435; 403968; 480167; 478701; 476060; 494000; 484056; 480621; 481330; 484496; 403477; 487119; 475603; 488487; 477955; 478040; 486242; 478922;
481521; 611876
dog subset 3 (hg)
477699; 491292; 480052; 477023; 483322; 478321; 488796; 478489; 403817; 403961; 403960; 403410; 490385; 403828; 403168; 403835; 490878; 474808; 403550;
610941; 483021; 403736; 481685; 403656; 607616; 475680; 476438; 486469; 442968; 403588; 475176; 474823; 403954; 486545; 474503; 479450; 607705; 449028;
403416; 403618; 403658; 403461; 403762; 486127; 490695; 491750; 403437; 475526; 482682; 478194; 476728; 482306; 474684; 442947; 403908; 403567; 488473;
403474; 403980; 487746; 485187; 486564; 607076; 403646; 403765; 487438; 612608; 415130; 403756; 403469; 403468; 488642; 474391; 489476; 479329; 403934;
442858; 491325; 403429; 477165; 485972; 490628; 484791; 484790; 448802; 484789; 485618; 403651; 403824; 403826; 478835; 403496; 403668; 403467; 609516;
442963; 479866; 403856; 611795; 403923; 608246; 487649; 477032; 486077; 483662; 494010; 484494; 484494; 474177; 415122; 415120; 415128; 479740; 415127;
608452; 494003; 609307; 474873; 403904; 610668; 607439; 480472; 610935; 478767; 403701; 487984; 607887; 489117; 484199; 403424; 450187; 403862; 403506;
490431; 403657; 404306; 487421; 487870; 475590; 442980; 480466; 492129; 609166; 474578; 489830; 480113; 404002; 403883; 476460; 403640; 403169; 489287;
480516; 488940; 490153; 478711; 490261; 486102; 474468; 483378; 484634; 475472; 608459; 475582; 475474; 491749; 477295; 403727; 442938; 481464; 403604;
480076; 403603; 490345; 475819; 403845; 490792; 484445; 611696; 478232; 478969; 489375; 489224; 474624; 486400; 442957; 403943; 403511; 486197; 607528;
442961; 483312; 494007; 478047; 478575; 474819; 475596; 479912; 611366; 477556; 477329; 403831; 490630; 403441; 480348; 475781; 442987; 490657; 485742;
484434; 478450; 403812; 475124; 480438; 478854; 612371; 403963; 481689; 403975; 480475; 609779; 481558; 403801; 610255; 442951; 483664; 480812; 488516;
491618; 610316; 608559; 481051; 482839; 403782; 403974; 481328; 403660; 403989; 403870; 403851; 403785; 403985; 612271; 403545; 612582; 403850; 478906;
478905; 611642; 403628; 610823; 611309; 403976; 442990; 481837; 403796; 483245; 478432; 483665; 484990; 478800; 481500; 486148; 489208; 488429; 486493;
477021; 489905; 489928; 488437; 489929; 477956; 403788; 483318; 608977; 488368; 477598; 484185; 609429; 609409; 403984; 482154; 403811; 478666; 442953;
479680; 478151; 491320; 403616; 404021; 403449; 488896; 481124; 481712; 475720; 486728; 480144; 610902; 476196; 611884; 484066; 480218; 403537; 481826;
490497; 403438; 403507; 490008; 475461; 489428; 403733; 403445; 489432; 489429; 403885; 486408; 403823; 487114; 491924; 403768; 804479; 448784; 403744;
403924; 477024; 490344; 611940; 481122; 487403; 607700; 485677; 483925; 485930; 442859; 486858; 488958; 480558; 403822; 403784; 480676; 475083; 484571;
608289; 403872; 478987; 608778; 482025; 484041; 611921; 609301; 403476; 612021; 491597; 442986; 610775; 611276; 475227; 611916; 476177; 481772; 607090;
606755; 480048; 482840; 403426; 476446; 488167; 479473; 403602; 489816; 481756; 403606; 482196; 481945; 489571; 490904; 489968; 494005; 609934; 477575;
486758; 477746; 612821; 478347; 611397; 488241; 476104; 478703; 403832; 403797; 403589; 442942; 403987; 403779; 475108; 475509; 490563; 477488; 490255;
486710; 403955; 481656; 479158; 476915; 478127; 483729; 484454; 403838; 403494; 478328; 480580; 490463; 480749; 476879; 448788; 403981; 480600; 448786;
403522; 491148; 448792; 445451; 403586; 448790; 611927; 487265; 482986; 477945; 449622; 403999; 480080; 403994; 484763; 489508; 475471; 403647; 490439;
483777; 403561; 483066; 403559; 476258; 486507; 488405; 612159; 485864; 403475; 612861; 403953; 489293; 475588; 488449; 481111; 490967; 403892; 475239;
485079; 477072; 488213; 403488; 403998; 488596; 490387; 481519; 481628; 485867; 474355; 487486; 484087; 607489; 477841; 482535; 403816; 403633; 481289;
448807; 403417; 488605; 403922; 403634; 487437; 403869; 480673; 490867; 480019; 403968; 480167; 478701; 490339; 476060; 610057; 403982; 486588; 403802; 482932; 494000; 484056; 484038; 480621;
399544; 481330; 483900; 484762; 483724; 484496; 612125; 403477; 487119; 485022; 609418; 490006; 475603; 485548; 489632; 488487; 477955; 488471; 403482; 481708; 483329; 477302; 475024; 442949;
480450; 484802; 479307; 403625; 611678; 488651; 487323; 481688; 488653; 612928; 483625; 403702; 481396; 485848; 482271; 479684; 445454; 403958; 404018; 480322; 490608; 404017; 486124; 478922;
490187; 611869; 608988; 475467; 489925; 486549; 490265; 478024; 607575; 478007; 475533; 486674; 610101; 484401; 612306; 608333; 480847; 609866; 477767; 477112; 490113; 611876; 608593;
612543; 612539
rat subset 1 (hg)
24248; 25634; 25585; 25586; 362282; 25256; 113992; 291863; 60582; 25697; 286918; 24179; 24567; 83785; 116510; 25661; 81687; 83783; 312903; 295235; 361233;
84348; 25464; 287562; 24498; 80843; 171522; 29296; 25086; 361523; 64524; 25703
rat subset 2 (hg)
81638; 24182; 296654; 24434; 24484; 25704; 140924; 24499; 25205; 54258; 29385; 360606; 311144; 315346; 308995; 25021; 309684; 311061; 25713; 245955; 59107;
29409; 25153; 299996; 25684; 81687; 81521; 299766; 81523; 337868; 81736; 81526; 24618; 24650; 363633; 29441; 84400; 58826; 24695; 25622; 309165; 116637; 25216;
25615; 24772; 310552; 78968; 24806; 84382; 171046; 116487; 56083; 292406; 25358; 497832; 60395; 84494; 140933; 89823; 25360; 24856; 498736; 25575; 24874;
54319; 29169; 117022; 81510; 364786; 290794; 81527; 246331; 84578; 79252; 360801; 301529; 53372; 282822; 367455
rat subset 3 (hg)
24153; 311860; 304883; 501072; 50681; 29650; 24170; 24172; 25026; 29290; 25369; 29316; 25591; 58968; 81722; 81638; 24182; 24185; 25374; 24186; 266802; 64621;
301585; 24197; 25377; 89807; 89805; 29339; 54225; 25728; 292855; 246097; 25385; 24208; 29183; 29221; 117273; 315305; 79255; 300711; 29757; 58919; 24224; 24888;
25245; 24225; 25295; 308755; 83470; 25296; 367786; 497672; 114486; 64022; 298566; 116506; 81715; 316010; 29144; 25402; 64044; 24248; 25404; 367218; 50662;
29345; 315291; 60350; 25660; 25408; 497809; 305081; 25668; 171369; 84349; 25406; 245962; 29186; 287435; 25599; 64465; 83502; 362110; 83571; 24252; 24253;
24258; 24259; 289032; 25590; 25627; 57301; 60463; 117027; 117029; 140473; 29393; 84352; 25412; 84032; 290905; 306628; 294337; 361821; 301012; 499431; 497663;
54245; 25419; 81649; 78965; 116630; 25610; 315707; 29317; 64032; 84353; 64529; 171293; 24296; 24297; 361523; 29296; 293985; 246070; 29277; 171522; 25086;
171352; 498198; 24307; 25147; 64036; 140926; 29139; 24310; 29467; 287765; 24314; 25253; 24318; 29238; 114856; 315219; 306792; 24323; 24326; 50672; 171361;
94268; 25313; 24329; 366492; 117045; 116636; 287986; 299606; 363854; 302811; 25043; 29452; 25315; 24335; 24337; 292747; 293701; 24356; 303547; 29251; 116677;
25584; 290757; 60327; 289055; 50646; 315191; 282583; 171075; 24366; 54250; 60662; 24367; 360457; 84482; 54251; 499148; 497666; 25256; 246245; 246247; 246248;
25661; 314322; 308411; 292413; 56718; 317385; 29573; 25453; 297417; 81664; 60429; 60449; 363682; 24404; 81504; 24408; 24413; 114105; 84027; 296654; 116686;
24422; 24424; 29438; 25260; 24434; 171528; 24446; 29560; 25059; 24451; 25098; 25099; 25100; 79256; 25461; 299331; 497838; 25462; 313641; 25323; 25464; 360647;
25261; 25587; 298205; 24481; 25712; 24482; 25718; 24483; 25685; 25662; 24484; 25285; 289560; 302822; 84351; 24493; 24494; 25663; 60582; 116562; 25704; 140924;
287287; 24498; 24499; 25205; 294797; 54258; 29385; 24500; 25325; 117539; 360303; 64546; 116553; 301289; 29197; 29200; 245920; 24506; 24954; 114517; 24508;
25467; 170921; 360606; 311144; 315346; 308995; 25021; 296456; 499271; 24511; 309684; 25724; 311061; 25713; 24514; 24516; 24518; 83731; 64030; 288001; 305078;
24530; 313050; 170496; 25608; 83781; 60584; 171105; 299858; 361795; 59107; 297604; 29357; 25631; 50554; 29409; 25153; 291439; 60430; 29333; 25684; 60427;
85483; 81683; 246759; 291234; 25672; 300339; 81686; 171045; 25335; 63849; 81687; 117061; 25481; 81707; 291327; 81521; 26198; 24577; 301059; 81523; 337868;
314910; 50594; 114553; 300955; 307231; 311658; 81736; 309452; 25494; 191575; 24599; 24600; 25496; 81526; 64563; 24602; 24605; 360265; 192281; 25341; 25601;
289747; 25504; 24617; 24618; 64513; 25266; 287526; 500006; 94203; 116589; 24649; 24650; 24653; 25692; 25619; 25096; 25097; 85253; 29441; 25682; 25664; 246358;
83471; 58826; 78975; 24680; 25023; 170538; 29340; 116590; 50689; 29513; 170851; 24683; 25268; 117263; 50557; 81752; 84023; 29527; 24695; 56813; 25614; 24924;
117063; 25622; 24699; 312703; 363875; 298012; 25676; 24708; 81758; 309165; 365510; 24715; 24716; 192264; 300807; 83840; 94195; 287562; 116637; 81780; 287561;
29397; 24770; 117518; 29538; 117551; 60665; 89808; 25216; 25615; 24772; 25544; 29259; 25651; 315996; 310552; 29517; 85385; 50567; 81826; 64347; 24786; 24787;
24790; 367846; 24791; 83805; 78968; 501099; 116650; 315548; 25557; 25124; 288774; 25125; 362896; 83783; 24806; 171046; 89804; 297758; 306862; 25563; 59086;
81809; 289316; 116487; 29591; 56083; 83580; 292406; 81811; 361655; 89806; 116510; 29543; 25358; 310553; 29260; 289337; 24835; 25625; 497808; 24842; 311786;
362788; 311245; 25360; 24856; 498736; 89814; 116484; 25575; 83808; 58851; 25361; 24873; 83785; 114111; 24874; 54319; 117064; 29169; 116669; 117022; 60628;
363142; 25445; 81510; 117274; 364786; 83807; 171137; 117516; 252971; 290794; 29376; 500592; 301365; 81527; 246331; 117279; 84385; 294235; 170897; 295052; 294568; 89829; 64520; 498609; 64193;
246253; 64565; 66015; 79252; 116673; 252878; 65035; 60351; 58852; 81732; 289820; 362248; 29374; 83726; 362505; 50993; 298068; 302248; 84551; 367253; 306009; 301529; 360355; 311783; 117089;
117108; 80881; 60382; 282817; 315298; 363983; 288378; 293469; 311332; 24861; 361273; 286938; 312800; 308589; 170919; 298646; 302668; 192206; 316561; 309512; 85251; 502531; 192205; 367455;
315388; 316758
mouse subset 1 (hg)
12359; 14377; 15903; 11647; 18534; 14262; 14261; 394431; 104158; 16181; 13039; 17858; 11606; 71755; 17748; 22339; 21857; 14268; 17395; 20887; 72265; 66256;
107513; 12778; 15894; 20293; 16193; 53421; 56448; 13098; 13106; 11814; 13088; 21881; 19662
mouse subset 2 (hg)
11608; 11609; 227753; 110006; 21923; 16009; 16184; 16186; 16194; 16195; 227288; 12765; 16400; 16401; 16402; 16408; 16409; 16414; 16420; 16421; 19039; 268977;
17136; 17171; 17181; 17287; 17288; 17395; 17698; 17877; 17926; 17927; 18033; 18133; 18489; 18507; 18763; 20020; 18984; 116847; 19074; 19227; 19247; 19697;
20303; 20969; 15529; 20315; 20319; 20787; 21333; 21413; 21423; 21810; 21817; 21825; 21826; 21859; 22063; 22065; 22066; 22067; 22068; 22095; 22139; 22151; 22287;
22346; 22350; 22370; 16178; 12587; 53614; 241226; 21951; 18187; 18186; 15183; 11504; 20602; 71828; 13437; 269113; 26946; 76905
mouse subset 3 (hg)
232345; 11350; 11352; 113868; 11430; 11487; 11518; 11522; 11535; 11539; 11540; 11541; 11545; 11595; 11596; 11608; 11609; 11651; 17025; 11657; 11669; 11682;
11648; 11650; 76768; 11699; 11727; 11600; 11601; 20219; 238055; 11816; 16613; 14102; 14103; 11835; 11839; 11846; 11848; 11908; 11911; 11920; 11975; 12443;
12043; 12048; 12062; 12064; 12097; 12144; 12153; 12159; 12169; 12189; 109880; 12223; 12259; 12266; 12311; 12328; 12796; 12330; 12367; 12370; 12359; 12389;
12393; 12406; 12455; 12475; 12487; 12519; 21941; 21949; 12490; 12494; 21939; 21947; 12505; 12506; 12512; 12514; 16149; 12540; 12550; 107951; 12576; 12606;
12608; 12652; 12653; 71884; 11438; 17228; 12768; 12772; 12771; 12774; 59289; 12842; 12843; 12824; 12825; 12826; 12827; 12833; 12834; 12836; 12817; 12870; 12914;
12928; 12944; 26416; 12977; 12981; 12985; 12988; 13008; 14219; 12387; 13030; 13033; 13076; 13077; 13088; 13098; 72303; 13095; 72082; 56448; 13106; 13113;
337924; 13120; 13075; 13136; 13163; 13179; 11421; 13198; 13207; 18104; 13482; 13489; 13490; 19252; 72962; 13610; 13614; 13617; 13618; 13627; 13636; 13645;
13649; 13836; 13684; 13685; 208643; 50701; 15568; 56501; 13717; 13805; 13819; 13846; 13849; 13856; 13866; 14113; 13982; 26379; 23871; 14055; 14061; 14063;
14066; 109821; 74145; 14060; 19229; 14114; 14104; 14115; 14128; 110135; 14173; 14176; 14182; 99571; 14205; 56458; 14254; 14256; 14257; 14261; 55990; 14262;
226564; 14263; 14268; 14281; 14282; 14289; 56717; 233908; 13349; 14385; 14573; 14583; 14598; 14678; 14683; 14695; 14733; 12777; 14775; 14784; 14810; 14815;
20310; 56637; 227753; 14782; 14863; 14871; 110006; 14939; 15234; 15251; 15277; 15368; 15375; 15376; 15377; 15466; 15499; 15519; 15510; 15528; 15552; 21923;
15894; 15896; 15901; 15902; 15974; 15977; 15978; 16000; 16001; 16002; 16006; 16008; 16009; 16011; 29817; 209268; 16150; 16175; 16176; 16177; 16181; 16183;
16184; 16186; 16189; 16193; 16194; 16195; 16196; 16197; 227288; 12765; 16199; 16153; 16154; 16156; 16160; 16163; 16171; 16173; 16323; 15945; 16334; 16403;
16362; 16367; 16398; 16400; 16401; 16402; 104099; 16408; 16409; 16410; 16411; 16412; 16414; 16416; 192897; 16419; 16420; 16421; 16452; 16476; 16478; 16531;
16542; 16590; 16644; 16780; 16816; 16818; 16819; 16846; 16854; 16878; 240028; 16971; 16994; 268977; 17000; 17126; 17127; 17128; 17136; 17171; 17190; 17210;
17221; 17287; 17288; 17295; 17311; 12265; 545943; 17329; 17345; 110784; 83995; 17390; 17392; 17393; 17394; 17395; 17384; 17385; 17387; 17533; 17698; 17750;
17709; 17829; 17869; 17874; 17926; 17927; 17937; 17965; 17969; 17973; 18018; 18019; 18033; 18034; 18073; 18102; 18126; 18127; 18128; 18133; 54631; 230899;
18176; 18214; 246730; 18383; 18390; 18413; 18429; 18787; 18489; 114774; 18507; 18590; 18613; 20317; 71382; 18654; 20724; 18712; 69713; 18763; 18783; 18791;
18792; 18793; 18805; 18806; 18815; 18984; 19015; 19016; 57349; 19072; 19074; 108079; 18750; 18751; 18753; 18754; 19091; 26413; 26417; 26419; 26415; 26395;
19106; 19109; 19123; 19179; 19211; 19217; 19219; 19225; 19227; 19228; 14083; 19242; 19255; 19247; 19264; 19305; 19353; 19359; 218397; 19645; 19651; 19697;
19698; 19702; 19713; 93726; 19883; 20190; 20202; 20208; 20210; 20293; 20303; 20304; 20306; 20292; 20296; 20295; 20297; 20299; 20311; 20312; 20969; 15529; 20315;
20339; 20343; 20344; 20350; 26398; 20319; 20393; 20416; 17254; 56643; 20515; 20618; 545845; 20656; 20683; 20687; 20692; 20779; 20787; 20807; 24067; 67398;
20845; 20846; 20847; 20848; 20852; 20887; 21333; 21423; 21687; 21749; 22041; 21418; 21786; 21803; 21808; 320202; 21810; 21812; 21817; 21824; 21825; 21826;
21832; 21843; 21846; 21857; 21858; 21859; 110595; 24088; 21898; 53791; 21926; 21937; 21938; 22059; 22030; 22031; 22033; 22034; 22095; 22139; 22151; 22164;
22166; 22287; 231396; 22260; 22329; 22337; 22339; 22340; 22341; 22346; 22350; 22353; 22370; 22371; 16178; 12767; 20347; 14283; 12587; 23957; 53614; 241226; 56449; 21943; 12703; 21951; 384783; 85030;
16182; 18187; 18186; 12633; 18171; 15937; 20698; 12427; 83767; 12702; 116870; 53978; 30939; 11450; 231382; 240913; 11504; 20970; 326623; 12355; 20186; 22259; 74117; 66713; 19124; 12464; 13018; 20301;
19325; 66212; 71828; 18074; 50934; 20335; 213053; 13437; 65086; 54450; 12070; 76898; 234836; 28146; 99889; 66824; 26934; 329244; 69165; 67869; 54485; 57743; 107526; 54343; 56632; 68262; 11610; 70008;
50501; 77629; 74493; 12822; 229317; 246691; 76905; 71753; 338374; 16772

TABLE 4
particularly preferred human toxicity markers

| subset 1; | | |
|---|---|---|
| 2 way, not b | | |
| 972 | CD74 | CD74 antigen (invariant polypeptide of major histocompatibility complex, class II antigen-associated) |
| 1543 | CYP1A1 | cytochrome P450, family 1, subfamily A, polypeptide 1 |
| 1728 | NQO1 | NAD(P)H dehydrogenase, quinone 1 |
| 2052 | EPHX1 | epoxide hydrolase 1, microsomal (xenobiotic) |
| 2194 | FASN | fatty acid synthase |
| 2538 | G6PC | glucose-6-phosphatase, catalytic (glycogen storage disease type I, von Gierke disease) |
| 2729 | GCLC | glutamate-cysteine ligase, catalytic subunit |
| 3120 | HLA-DQB2 | major histocompatibility complex, class II, DQ beta 2 |
| 3127 | HLA-DRB5 | major histocompatibility complex, class II, DR beta 5 |
| 4501 | MT1X | metallothionein 1X |
| 4942 | OAT | ornithine aminotransferase (gyrate atrophy) |
| 5105 | PCK1 | phosphoenolpyruvate carboxykinase 1 (soluble) |
| 5207 | PFKFB1 | 6-phosphofructo-2-kinase/fructose-2,6-biphosphatase 1 |
| 6799 | SULT1A2 | sulfotransferase family, cytosolic, 1A, phenol-preferring, member 2 |
| 7076 | TIMP1 | TIMP metallopeptidase inhibitor 1 |
| 7086 | TKT | transketolase (Wernicke-Korsakoff syndrome) |
| 54578 | UGT1A6 | UDP glucuronosyltransferase 1 family, polypeptide A6 |
| subset 2; | | |
| 3 way wb | | |
| 972 | CD74 | CD74 antigen (invariant polypeptide of major histocompatibility complex, class II antigen-associated) |
| 1543 | CYP1A1 | cytochrome P450, family 1, subfamily A, polypeptide 1 |
| 1728 | NQO1 | NAD(P)H dehydrogenase, quinone 1 |
| 2538 | G6PC | glucose-6-phosphatase, catalytic (glycogen storage disease type I, von Gierke disease) |
| 3120 | HLA-DQB2 | major histocompatibility complex, class II, DQ beta 2 |
| 4501 | MT1X | metallothionein 1X |
| 4942 | OAT | ornithine aminotransferase (gyrate atrophy) |
| 5105 | PCK1 | phosphoenolpyruvate carboxykinase 1 (soluble) |
| 5207 | PFKFB1 | 6-phosphofructo-2-kinase/fructose-2,6-biphosphatase 1 |
| 6799 | SULT1A2 | sulfotransferase family, cytosolic, 1A, phenol-preferring, member 2 |
| 7076 | TIMP1 | TIMP metallopeptidase inhibitor 1 |
| 7086 | TKT | transketolase (Wernicke-Korsakoff syndrome) |
| 54578 | UGT1A6 | UDP glucuronosyltransferase 1 family, polypeptide A6 |
| subset 3; | | |
| 2 way, with b | | |
| 47 | ACLY | ATP citrate lyase |
| 249 | ALPL | alkaline phosphatase, liver/bone/kidney |
| 388 | RHOB | ras homolog gene family, member B |
| 595 | CCND1 | cyclin D1 |
| 635 | BHMT | betaine-homocysteine methyltransferase |
| 847 | CAT | catalase |
| 900 | CCNG1 | cyclin G1 |
| 972 | CD74 | CD74 antigen (invariant polypeptide of major histocompatibility complex, class II antigen-associated) |
| 1026 | CDKN1A | cyclin-dependent kinase inhibitor 1A (p21, Cip1) |
| 1515 | CTSL2 | cathepsin L2 |
| 1543 | CYP1A1 | cytochrome P450, family 1, subfamily A, polypeptide 1 |
| 1555 | CYP2B6 | cytochrome P450, family 2, subfamily B, polypeptide 6 |
| 1565 | CYP2D6 | cytochrome P450, family 2, subfamily D, polypeptide 6 |
| 1571 | CYP2E1 | cytochrome P450, family 2, subfamily E, polypeptide 1 |
| 1634 | DCN | decorin |
| 1727 | CYB5R3 | cytochrome b5 reductase 3 |
| 1728 | NQO1 | NAD(P)H dehydrogenase, quinone 1 |
| 2052 | EPHX1 | epoxide hydrolase 1, microsomal (xenobiotic) |
| 2194 | FASN | fatty acid synthase |
| 2212 | FCGR2A | Fc fragment of IgG, low affinity IIa, receptor (CD32) |
| 2224 | FDPS | farnesyl, diphosphate synthase (farnesyl pyrophosphate synthetase, dimethylallyltranstransferase, geranyltranstransferase) |
| 2326 | FMO1 | flavin containing monooxygenase 1 |
| 2538 | G6PC | glucose-6-phosphatase, catalytic (glycogen storage disease type I, von Gierke disease) |
| 2539 | G6PD | glucose-6-phosphate dehydrogenase |
| 2645 | GCK | glucokinase (hexokinase 4, maturity onset diabetes of the young 2) |
| 2729 | GCLC | glutamate-cysteine ligase, catalytic subunit |
| 2805 | GOT1 | glutamic-oxaloacetic transaminase 1, soluble (aspartate aminotransferase 1) |
| 2936 | GSR | glutathione reductase |
| 2940 | GSTA3 | glutathione S-transferase A3 |
| 3120 | HLA-DQB2 | major histocompatibility complex, class II, DQ beta 2 |
| 3127 | HLA-DRB5 | major histocompatibility complex, class II, DR beta 5 |
| 3157 | HMGCS1 | 3-hydroxy-3-methylglutaryl-Coenzyme A synthase 1 (soluble) |
| 3162 | HMOX1 | heme oxygenase (decycling) 1 |
| 3294 | HSD17B2 | hydroxysteroid (17-beta) dehydrogenase 2 |
| 3383 | ICAM1 | intercellular adhesion molecule 1 (CD54), human rhinovirus receptor |
| 3399 | ID3 | inhibitor of DNA binding 3, dominant negative helix-loop-helix protein |
| 3627 | CXCL10 | chemokine (C—X—C motif) ligand 10 |
| 4023 | LPL | lipoprotein lipase |
| 4193 | MDM2 | Mdm2, transformed 3T3 cell double minute 2, p53 binding protein (mouse) |
| 4501 | MT1X | metallothionein 1X |
| 4609 | MYC | v-myc myelocytomatosis viral oncogene homolog (avian) |
| 4942 | OAT | ornithine aminotransferase (gyrate atrophy) |
| 5009 | OTC | ornithine carbamoyltransferase |
| 5105 | PCK1 | phosphoenolpyruvate carboxykinase 1 (soluble) |
| 5207 | PFKFB1 | 6-phosphofructo-2-kinase/fructose-2,6-biphosphatase 1 |
| 5950 | RBP4 | retinol binding protein 4, plasma |
| 6307 | SC4MOL | sterol-C4-methyl oxidase-like |
| 6319 | SCD | stearoyl-CoA desaturase (delta-9-desaturase) |
| 6799 | SULT1A2 | sulfotransferase family, cytosolic, 1A, phenol-preferring. member 2 |
| 6817 | SULT1A1 | sulfotransferase family, cytosolic, 1A, phenol-preferring, member 1 |
| 6890 | TAP1 | transporter 1, ATP-binding cassette, sub-family B (MDR/TAP) |
| 6898 | TAT | tyrosine aminotransferase |
| 7076 | TIMP1 | TIMP metallopeptidase inhibitor 1 |
| 7086 | TKT | transketolase (Wernicke-Korsakoff syndrome) |
| 7276 | TTR | transthyretin (prealbumin, amyloidosis type I) |
| 7431 | VIM | vimentin |
| 7832 | BTG2 | BTG family, member 2 |
| 10993 | SDS | serine dehydratase |
| 22977 | AKR7A3 | aldo-keto reductase family 7, member A3 (aflatoxin aldehyde reductase) |
| 54578 | UGT1A6 | UDP glucuronosyltransferase 1 family, polypeptide A6 |

TABLE 5A
primate toxicology biomarkers
human subset 1 (f)

| | | |
|---|---|---|
| 302 | ANXA2 | annexin A2 |
| 55326 | AGPAT5 | 1-acylglycerol-3-phosphate O-acyltransferase 5 (lysophosphatidic acid acyltransferase, epsilon) |
| 5644 | PRSS1 | protease, serine, 1 (trypsin 1) |
| 65018 | PINK1 | PTEN induced putative kinase 1 |
| 5319 | PLA2G1B | phospholipase A2, group IB (pancreas) |
| 342898 | SYCN | syncollin |
| 714 | C1QC | complement component 1, q subcomponent, C chain |
| 5745 | PTHR1 | parathyroid hormone receptor 1 |
| 10630 | PDPN | podoplanin |
| 627 | BDNF | brain-derived neurotrophic factor |
| 23102 | TBC1D2B | TBC1 domain family, member 2B |
| 9289 | GPR56 | G protein-coupled receptor 56 |
| 3394 | IRF8 | interferon regulatory factor 8 |
| 1357 | CPA1 | carboxypeptidase A1 (pancreatic) |
| 3767 | KCNJ11 | potassium inwardly-rectifying channel, subfamily J, member 11 |
| 10743 | RAI1 | retinoic acid induced 1 |
| 23597 | ACOT9 | acyl-CoA thioesterase 9 |
| 80148 | PQLC1 | PQ loop repeat containing 1 |
| 3835 | KIF22 | kinesin family member 22 |
| 64981 | MRPL34 | mitochondrial ribosomal protein L34 |
| 10563 | CXCL13 | chemokine (C—X—C motif) ligand 13 (B-cell chemoattractant) |
| 114880 | OSBPL6 | oxysterol binding protein-like 6 |
| 23109 | DDN | dendrin |
| 60485 | SAV1 | salvador homolog 1 (Drosophila) |
| 55287 | TMEM40 | transmembrane protein 40 |
| 6391 | SDHC | succinate dehydrogenase complex, subunit C, integral membrane protein, 15 kDa |
| 57619 | SHRM | shroom |
| 55671 | KIAA2010 | KIAA2010 |
| 10055 | SAE1 | SUMO-1 activating enzyme subunit 1 |
| 81847 | RNF146 | ring finger protein 146 |
| 1950 | EGF | epidermal growth factor (beta-urogastrone) |
| 9670 | IPO13 | importin 13 |
| 6539 | SLC6A12 | solute carrier family 6 (neurotransmitter transporter, betaine/GABA), member 12 |
| 3839 | KPNA3 | karyopherin alpha 3 (importin alpha 4) |
| 55847 | C10orf70 | chromosome 10 open reading frame 70 |
| 144110 | TMEM86A | transmembrane protein 86A |
| 11146 | GLMN | glomulin, FKBP associated protein |
| 83541 | C20orf55 | chromosome 20 open reading frame 55 |
| 34 | ACADM | acyl-Coenzyme A dehydrogenase, C-4 to C-12 straight chain |
| 23564 | DDAH2 | dimethylarginine dimethylaminohydrolase 2 |
| 374618 | TEX9 | testis expressed sequence 9 |
| 3043 | HBB | hemoglobin, beta |
| 3048 | HBG2 | hemoglobin, gamma G |
| 3046 | HBE1 | hemoglobin, epsilon 1 |
| 3045 | HBD | hemoglobin, delta |
| 79345 | OR51B2 | olfactory receptor, family 51, subfamily B, member 2 |
| 3047 | HBG1 | hemoglobin, gamma A |
| 51097 | SCCPDH | saccharopine dehydrogenase (putative) |
| 401 | PHOX2A | paired-like (aristaless) homeobox 2a |
| 4501 | MT1X | metallothionein 1X |
| 84830 | C6orf105 | chromosome 6 open reading frame 105 |
| 55591 | VEZT | vezatin, adherens junctions transmembrane protein |
| 149466 | C1orf210 | chromosome 1 open reading frame 210 |
| 84833 | USMG5 | upregulated during skeletal muscle growth 5 homolog (mouse) |
| 23305 | ACSL6 | acyl-CoA synthetase long-chain family member 6 |
| 29968 | PSAT1 | phosphoserine aminotransferase 1 |
| 64598 | MOSPD3 | motile sperm domain containing 3 |
| 94107 | MGC14327 | hypothetical protein MGC14327 |
| 57136 | C20orf3 | chromosome 20 open reading frame 3 |
| 9125 | RQCD1 | RCD1 required for cell differentiation1 homolog (S. pombe) |
| 81609 | SNX27 | sorting nexin family member 27 |
| 4931 | NVL | nuclear VCP-like |
| 4942 | OAT | ornithine aminotransferase (gyrate atrophy) |
| 54795 | TRPM4 | transient receptor potential cation channel, subfamily M, member 4 |
| 4069 | LYZ | lysozyme (renal amyloidosis) |
| 1066 | CES1 | carboxylesterase 1 (monocyte/macrophage serine esterase 1) |
| 1066 | CES1 | carboxylesterase 1 (monocyte/macrophage serine esterase 1) |

human subset 2 (f)
350; 634; 1387; 1388; 1508; 1956; 2064; 2065; 2099; 2250; 2885; 2990; 3050; 3084; 3240; 3659; 3660; 4267; 4684; 4846; 4908; 4909; 4915; 4999; 5111; 5245; 5296; 5327; 5340; 5430; 5806; 6275; 6277;
6281; 6477; 6688; 6714; 6833; 7263; 7448; 7520; 8291; 9318; 9351; 9368; 9373; 9464; 10434; 10477; 11005; 23286; 29124; 51365
human subset 3 (f)
2; 60; 71; 142; 207; 301; 303; 304; 305; 308; 309; 350; 367; 408; 498; 506; 595; 634; 643; 654; 660; 685; 712; 713; 823; 861; 862; 867; 960; 999; 1017; 1019; 1027; 1052;
1080; 1104; 1213; 1230; 1231; 1277; 1312; 1358; 1359; 1360; 1361; 1368; 1385; 1387; 1388; 1406; 1432; 1436; 1490; 1495; 1499; 1508; 1511; 1621; 1748; 1814; 1845;
1956; 1964; 1991; 2000; 2002; 2006; 2017; 2060; 2064; 2065; 2067; 2099; 2101; 2113; 2120; 2147; 2150; 2158; 2159; 2180; 2181; 2182; 2194; 2247; 2249; 2250; 2252;
2261; 2271; 2280; 2288; 2353; 2475; 2516; 2534; 2539; 2549; 2597; 2623; 2668; 2697; 2833; 2885; 2891; 2932; 2990; 3039; 3040; 3043; 3045; 3046; 3047; 3048; 3049;
3050; 3067; 3082; 3084; 3131; 3148; 3240; 3479; 3481; 3486; 3549; 3553; 3558; 3565; 3569; 3576; 3592; 3593; 3659; 3660; 3663; 3667; 3725; 3764; 3837; 3840; 3841;
3868; 3933; 3934; 3952; 4057; 4058; 4116; 4150; 4224; 4232; 4233; 4308; 4353; 4489; 4493; 4494; 4495; 4496; 4499; 4500; 4502; 4647; 4666; 4684; 4738; 4763; 4803;
4828; 4846; 4902; 4904; 4905; 4908; 4909; 4915; 4943; 4950; 5007; 5068; 5077; 5080; 5111; 5245; 5264; 5265; 5271; 5294; 5296; 5320; 5321; 5322; 5327; 5328; 5329;
5335; 5337; 5340; 5345; 5371; 5430; 5451; 5465; 5468; 5575; 5578; 5580; 5594; 5595; 5598; 5645; 5728; 5741; 5744; 5806; 5905; 5925; 5982; 5983; 5984; 5985; 6125;
6197; 6256; 6275; 6277; 6281; 6311; 6364; 6366; 6368; 6387; 6389; 6390; 6392; 6469; 6505; 6590; 6612; 6613; 6662; 6667; 6688; 6690; 6714; 6720; 6772; 6774; 6777;
6788; 6789; 6833; 6908; 6929; 6957; 7020; 7032; 7039; 7040; 7042; 7052; 7099; 7124; 7157; 7181; 7182; 7223; 7263; 7301; 7302; 7329; 7341; 7411; 7415; 7422; 7428;
7450; 7515; 7532; 7852; 7879; 8290; 8398; 8399; 8530; 8554; 8720; 8839; 8929; 9113; 9133; 9318; 9351; 9365; 9368; 9373; 9464; 9545; 9820; 9885; 9939; 10054; 10060; 10096; 10097; 10379; 10417; 10434;
10477; 10499; 10664; 10817; 10891; 10920; 11005; 22803; 22925; 22974; 23286; 23435; 23491; 23576; 23607; 23633; 26031; 26524; 26958; 29124; 51138; 51365; 51703; 51765; 55503; 56180; 56302; 56894;
56895; 56972; 57223; 58160; 80325; 81537; 93979; 114876; 114879; 114881; 146691; 154754; 158747; 170302; 171515; 192343; 497258
chimpanzee subset 1 (f)
453487; 463969; 463797; 456486; 456612; 461008; 456453; 453563; 454294; 463728; 466450; 459775; 452899; 460183; 450605; 461235; 456159; 462985; 463347;
469311; 452722; 450472; 466461; 467691; 450978; 450979; 466374; 469737; 462435; 452138; 456818; 462044; 461061; 464542; 472464; 458145; 457299; 457771;
450803; 456197
chimpanzee subset 2 (f)
468459; 467891; 463993; 454636; 451980; 463079; 471190; 463443; 467848; 454233; 461629; 465979; 466782; 466931; 459868; 458081; 455205; 455855; 472748;
460807; 457320; 457306; 465153; 458229; 451056; 458803; 449614; 453417; 454865; 465026; 459792; 462173; 471729; 469081
chimpanzee subset 3 (f)
450133; 454959; 457785; 453202; 472953; 461466; 462198; 468459; 465680; 455397; 451999; 461250; 469214; 451317; 451690; 452025; 464163; 463674; 456685;
458655; 463726; 452582; 467891; 468934; 462643; 462187; 463003; 462100; 463993; 467413; 464827; 455109; 470885; 468271; 468637; 473591; 456850; 454636;
451980; 463079; 466651; 473366; 451158; 461800; 465887; 452679; 461631; 459974; 454986; 466692; 471190; 461067; 451748; 453047; 461516; 451783; 465619;
471566; 461570; 470895; 463443; 450978; 466374; 450979; 467848; 453425; 463503; 455140; 461606; 454233; 466318; 463396; 470652; 450200; 461472; 449565;
463288; 471205; 460816; 471723; 461629; 456893; 465337; 470982; 462963; 455042; 464914; 464764; 449638; 460325; 453350; 456864; 462745; 463729; 463671;
453284; 454105; 471221; 452434; 466782; 454566; 468680; 470311; 466931; 451213; 459349; 458081; 455205; 450310; 467541; 455461; 463649; 455855; 456587;
456588; 472748; 450103; 470996; 453736; 460178; 454037; 472555; 466142; 466447; 473393; 460807; 458980; 452715; 463467; 452533; 460918; 457025; 450110;
464834; 457320; 457306; 459991; 456526; 451547; 472586; 458605; 463937; 450101; 451939; 458229; 470721; 454679; 472829; 458275; 451056; 468650; 470096; 470399; 456060; 457742; 460199; 452544;
458803; 453352; 467865; 459882; 456094; 450130; 470919; 467715; 468050; 458271; 463060; 453478; 453417; 454865; 465026; 468721; 457983; 468823; 459792; 472005; 461140; 460043; 462173; 458157;
471729; 456987; 456709; 463296; 463730; 469081; 470894; 465870; 458595; 467479; 472515; 468500; 454481; 465505

human subset 1 (mp)

| | | |
|---|---|---|
| 1066 | CES1 | carboxylesterase 1 (monocyte/macrophage serine esterase 1) |
| 51081 | MRPS7 | mitochondrial ribosomal protein S7 |
| 6128 | RPL6 | ribosomal protein L6 |
| 440926 | LOC440926 | H3 histone, family 3A pseudogene |
| 6228 | RPS23 | ribosomal protein S23 |
| 6307 | SC4MOL | sterol-C4-methyl oxidase-like |
| 10449 | ACAA2 | acetyl-Coenzyme A acyltransferase 2 (mitochondrial 3-oxoacyl-Coenzyme A thiolase) |
| 6579 | SLCO1A2 | solute carrier organic anion transporter family, member 1A2 |
| 5604 | MAP2K1 | mitogen-activated protein kinase kinase 1 |
| 506 | ATP5B | ATP synthase, H+ transporting, mitochondrial F1 complex, beta polypeptide |
| 1565 | CYP2D6 | cytochrome P450, family 2, subfamily D, polypeptide 6 |
| 201294 | UNC13D | unc-13 homolog D (C. elegans) |
| 23558 | WBP2 | WW domain binding protein 2 |
| 2 | A2M | alpha-2-macroglobulin |
| 174 | AFP | alpha-fetoprotein |
| 213 | ALB | albumin |
| 226 | ALDOA | aldolase A, fructose-bisphosphate |
| 1544 | CYP1A2 | cytochrome P450, family 1, subfamily A, polypeptide 2 |
| 1580 | CYP4B1 | cytochrome P450, family 4, subfamily B, polypeptide 1 |
| 2266 | FGG | fibrinogen gamma chain |
| 2944 | GSTM1 | glutathione S-transferase M1 |
| 2949 | GSTM5 | glutathione S-transferase M5 |
| 3029 | HAGH | hydroxyacylglutathione hydrolase |
| 3162 | HMOX1 | heme oxygenase (decycling) 1 |
| 4363 | ABCC1 | ATP-binding cassette, sub-family C (CFTR/MRP), member 1 |
| 6138 | RPL15 | ribosomal protein L15 |
| 4953 | ODC1 | ornithine decarboxylase 1 |
| 6554 | SLC10A1 | solute carrier family 10 (sodium/bile acid cotransporter family), member 1 |
| 6678 | SPARC | secreted protein, acidic, cysteine-rich (osteonectin) |
| 12 | SERPINA3 | serpin peptidase inhibitor, clade A (alpha-1 antiproteinase, antitrypsin), member 3 |
| 7157 | TP53 | tumor protein p53 (Li-Fraumeni syndrome) |
| 1191 | CLU | clusterin |
| 6853 | SYN1 | synapsin I |
| 3290 | HSD11B1 | hydroxysteroid (11-beta) dehydrogenase 1 |
| 4069 | LYZ | lysozyme (renal amyloidosis) |
| 5816 | PVALB | parvalbumin |
| 23600 | AMACR | alpha-methylacyl-CoA racemase |
| 2180 | ACSL1 | acyl-CoA synthetase long-chain family member 1 |
| 341 | APOC1 | apolipoprotein C-I |
| 2052 | EPHX1 | epoxide hydrolase 1, microsomal (xenobiotic) |
| 7253 | TSHR | thyroid stimulating hormone receptor |
| 1401 | CRP | C-reactive protein, pentraxin-related |
| 5034 | P4HB | procollagen-proline, 2-oxoglutarate 4-dioxygenase (proline 4-hydroxylase), beta polypeptide |
| 5009 | OTC | ornithine carbamoyltransferase |
| 1515 | CTSL2 | cathepsin L2 |
| 3691 | ITGB4 | integrin, beta 4 |
| 22977 | AKR7A3 | aldo-keto reductase family 7, member A3 (aflatoxin aldehyde reductase) |
| 173 | AFM | afamin |
| 350 | APOH | apolipoprotein H (beta-2-glycoprotein I) |
| 79637 | ARMC7 | armadillo repeat containing 7 |
| 51155 | HN1 | hematological and neurological expressed 1 |
| 79902 | NUP85 | nucleoporin 85 kDa |
| 9400 | RECQL5 | RecQ protein-like 5 |
| 2584 | GALK1 | galactokinase 1 |
| 347733 | TUBB2B | tubulin, beta 2B |
| 7448 | VTN | vitronectin |
| 1066 | CES1 | carboxylesterase 1 (monocyte/macrophage serine esterase 1) |
| 203068 | TUBB | tubulin, beta |
| 2643 | GCH1 | GTP cyclohydrolase 1 (dopa-responsive dystonia) |
| 6189 | RPS3A | ribosomal protein S3A |
| 6194 | RPS6 | ribosomal protein S6 |
| 325 | APCS | amyloid P component, serum |
| 6157 | RPL27A | ribosomal protein L27a |
| 3303 | HSPA1A | heat shock 70 kDa protein 1A |
| 191 | AHCY | S-adenosylhomocysteine hydrolase |
| 3875 | KRT18 | keratin 18 |
| 1938 | EEF2 | eukaryotic translation elongation factor 2 |
| 2705 | GJB1 | gap junction protein, beta 1, 32 kDa (connexin 32, Charcot-Marie-Tooth neuropathy, X-linked) |
| 51382 | ATP6V1D | ATPase, H+ transporting, lysosomal 34 kDa, V1 subunit D |
| 60386 | SLC25A19 | solute carrier family 25 (mitochondrial deoxynucleotide carrier), member 19 |
| 9772 | KIAA0195 | KIAA0195 |
| 7050 | TGIF | TGFB-induced factor (TALE family homeobox) |
| 1397 | CRIP2 | cysteine-rich protein 2 |
| 23163 | GGA3 | golgi associated, gamma adaptin ear containing, ARF binding protein 3 |
| 57409 | MIF4GD | MIF4G domain containing |
| 3993 | LLGL2 | lethal giant larvae homolog 2 (Drosophila) |
| 29115 | SAP30BP | SAP30 binding protein |
| 85451 | ZC3H5 | zinc finger CCCH-type containing 5 |
| 2243 | FGA | fibrinogen alpha chain |
| 4076 | GPIAP1 | GPI-anchored membrane protein 1 |
| 127700 | C1orf102 | chromosome 1 open reading frame 102 |
| 736 | C9 | complement component 9 |
| 7280 | TUBB2A | tubulin, beta 2A |
| 2948 | GSTM4 | glutathione S-transferase M4 |
| 7430 | VIL2 | villin 2 (ezrin) |
| 3700 | ITIH4 | inter-alpha (globulin) inhibitor H4 (plasma Kallikrein-sensitive glycoprotein) |
| 302 | ANXA2 | annexin A2 |
| 2946 | GSTM2 | glutathione S-transferase M2 (muscle) |
| 435 | ASL | argininosuccinate lyase |
| 6124 | RPL4 | ribosomal protein L4 |
| 6152 | RPL24 | ribosomal protein L24 |
| 4942 | OAT | ornithine aminotransferase (gyrate atrophy) |
| 58473 | PLEKHB1 | pleckstrin homology domain containing, family B (evectins) member 1 |
| 6202 | RPS8 | ribosomal protein S8 |
| 3053 | SERPIND1 | serpin peptidase inhibitor, clade D (heparin cofactor), member 1 |
| 2885 | GRB2 | growth factor receptor-bound protein 2 |
| 4282 | MIF | macrophage migration inhibitory factor (glycosylation-inhibiting factor) |
| 1036 | CDO1 | cysteine dioxygenase, type I |
| 5127 | PCTK1 | PCTAIRE protein kinase 1 |
| 6125 | RPL5 | ribosomal protein L5 |
| 6146 | RPL22 | ribosomal protein L22 |
| 6205 | RPS11 | ribosomal protein S11 |
| 6281 | S100A10 | S100 calcium binding protein A10 (annexin II ligand, calpactin I, light polypeptide (p11)) |
| 60 | ACTB | actin, beta |
| 442245 | LOC442245 | glutathione S-transferase M1 pseudogene |
| 10399 | GNB2L1 | guanine nucleotide binding protein (G protein), beta polypeptide 2-like 1 |
| 400963 | LOC400963 | hypothetical gene supported by AB082925; BC019021; NM_002952 |

human subset 2 (mp)
70; 259; 348; 350; 351; 354; 529; 567; 634; 712; 722; 813; 966; 1191; 1215; 1234; 1401; 1409; 1504; 1506; 1508; 1511; 1773; 1990; 1991; 2010; 2064; 2153; 2161; 2209;
2212; 2213; 2214; 2217; 2244; 2266; 2314; 2335; 2537; 2547; 2644; 2706; 2820; 2885; 2886; 2889; 2934; 2946; 2947; 2990; 3020; 3064; 3075; 3183; 3192; 3265; 3273;
3309; 3337; 3383; 3553; 3560; 3586; 3635; 3655; 3667; 3674; 3684; 3690; 3691; 3692; 3702; 3716; 3717; 3791; 3815; 3817; 3855; 3931; 3952; 3953; 4035; 4036; 4133;
4217; 4233; 4261; 4267; 4481; 4485; 4486; 4582; 4687; 4803; 4811; 4842; 4892; 4914; 4950; 4952; 5004; 5027; 5047; 5058; 5104; 5111; 5144; 5148; 5155; 5159; 5201;
5216; 5217; 5245; 5287; 5295; 5296; 5310; 5318; 5327; 5335; 5337; 5338; 5339; 5340; 5345; 5411; 5422; 5444; 5552; 5573; 5581; 5610; 5621; 5716; 5747; 5757; 5764;
5770; 5777; 5781; 5782; 5786; 5788; 5791; 5830; 5879; 5880; 5894; 5913; 5921; 5950; 5979; 6159; 6196; 6198; 6275; 6277; 6281; 6300; 6352; 6402; 6442; 6452; 6461;
6464; 6523; 6597; 6625; 6640; 6654; 6655; 6662; 6709; 6714; 6722; 6774; 6850; 6853; 6854; 6927; 7010; 7038; 7040; 7046; 7048; 7052; 7057; 7132; 7133; 7157; 7158;
7159; 7169; 7170; 7187; 7251; 7265; 7275; 7280; 7295; 7319; 7329; 7337; 7341; 7399; 7409; 7410; 7422; 7430; 7436; 7448; 7454; 7462; 7486; 7490; 7528; 7529; 7531;
7532; 7533; 7534; 7707; 7804; 7874; 7916; 8029; 8038; 8178; 8224; 8291; 8467; 8471; 8516; 8554; 8555; 8556; 8575; 8605; 8626; 8631; 8649; 8651; 8655; 8660; 8717;
8728; 8751; 8794; 8795; 8805; 8850; 8871; 8904; 8936; 8974; 8976; 9013; 9014; 9015; 9020; 9101; 9112; 9131; 9185; 9191; 9219; 9232; 9344; 9351; 9368; 9401; 9419;
9440; 9510; 9530; 9531; 9532; 9540; 9564; 9649; 9656; 9672; 9712; 9722; 9804; 9846; 9868; 10019; 10040; 10049; 10055; 10075; 10095; 10153; 10163; 10188; 10210;
10252; 10253; 10273; 10399; 10434; 10451; 10461; 10474; 10498; 10527; 10529; 10576; 10657; 10672; 10694; 10817; 10818; 10825; 10848; 10915; 10963; 10987; 11184;
11186; 11187; 11244; 22821; 22895; 22927; 22948; 23022; 23049; 23059; 23113; 23118; 23162; 23214; 23513; 23607; 23640; 23645; 25898; 25932; 25942; 25970; 26191;
26287; 26354; 26578; 26986; 27036; 27040; 27240; 27250; 28996; 29124; 29760; 29967; 29979; 30001; 30008; 30011; 30837; 50484; 51147; 51206; 51246; 51365; 51517; 51604; 51726; 51741; 51754; 51762;
53358; 53405; 54464; 54700; 54812; 54822; 54900; 54971; 55140; 55207; 55257; 55333; 55503; 55824; 55914; 56605; 57085; 57147; 57504; 57787; 58480; 64215; 64326; 64386; 64756; 79797; 80306; 80854;
81928; 83874; 83988; 84172; 84289; 84962; 85415; 85461; 90070; 91647; 91768; 92799; 94241; 118460; 121504; 125168; 131034; 137735; 139716; 140890; 143425; 149428; 157310; 202559; 204851; 221184;
259215; 283208; 283455
human subset 3 (mp)
9; 10; 13; 25; 33; 43; 51; 58; 59; 60; 69; 70; 71; 73; 75; 82; 86; 87; 100; 125; 126; 135; 136; 142; 147; 154; 156; 164; 174; 185; 196; 207; 208; 210; 211; 213; 226; 228; 229;
230; 238; 239; 240; 242; 259; 273; 301; 302; 303; 304; 305; 308; 309; 310; 317; 324; 325; 328; 332; 336; 338; 339; 342; 344; 348; 350; 351; 354; 355; 356; 367; 368; 369;
375; 377; 383; 387; 388; 396; 397; 409; 427; 432; 433; 467; 472; 498; 506; 507; 508; 522; 523; 529; 545; 558; 567; 571; 572; 573; 578; 580; 581; 590; 595; 596; 597; 598;
602; 604; 613; 632; 634; 637; 641; 644; 654; 665; 666; 667; 672; 673; 675; 705; 712; 718; 722; 727; 729; 730; 731; 732; 735; 768; 800; 805; 811; 813; 821; 823; 834; 836;
839; 840; 841; 842; 847; 857; 861; 862; 867; 868; 890; 891; 898; 908; 928; 933; 939; 940; 958; 960; 965; 966; 971; 972; 973; 977; 983; 990; 995; 999; 1003; 1017; 1019;
1020; 1021; 1026; 1027; 1029; 1030; 1048; 1050; 1051; 1071; 1072; 1080; 1081; 1107; 1111; 1131; 1147; 1191; 1207; 1213; 1215; 1231; 1234; 1244; 1263; 1277; 1278;
1305; 1308; 1380; 1385; 1386; 1387; 1398; 1399; 1401; 1409; 1432; 1436; 1437; 1439; 1441; 1442; 1445; 1452; 1453; 1454; 1457; 1459; 1460; 1462; 1466; 1475; 1487;
1490; 1499; 1504; 1506; 1508; 1510; 1511; 1513; 1523; 1535; 1537; 1543; 1544; 1545; 1551; 1555; 1557; 1558; 1559; 1562; 1564; 1565; 1568; 1571; 1574; 1576; 1577;
1580; 1583; 1586; 1601; 1605; 1612; 1616; 1630; 1639; 1643; 1647; 1649; 1652; 1655; 1662; 1667; 1673; 1718; 1728; 1730; 1739; 1758; 1759; 1773; 1786; 1813; 1814;
1820; 1825; 1832; 1834; 1840; 1843; 1844; 1854; 1869; 1877; 1938; 1950; 1956; 1958; 1969; 1977; 1978; 1981; 1984; 1990; 1991; 1994; 2000; 2001; 2006; 2010; 2017;
2033; 2052; 2053; 2057; 2064; 2065; 2068; 2071; 2074; 2099; 2113; 2114; 2119; 2146; 2147; 2152; 2153; 2159; 2161; 2162; 2165; 2168; 2176; 2181; 2182; 2185; 2205;
2209; 2212; 2213; 2214; 2217; 2243; 2244; 2247; 2252; 2260; 2261; 2263; 2266; 2272; 2274; 2309; 2314; 2317; 2321; 2322; 2324; 2335; 2353; 2475; 2492; 2520; 2534;
2537; 2547; 2549; 2585; 2596; 2597; 2626; 2644; 2670; 2671; 2688; 2690; 2697; 2706; 2719; 2729; 2735; 2776; 2778; 2782; 2810; 2820; 2861; 2874; 2885; 2886; 2887;
2888; 2908; 2911; 2932; 2934; 2938; 2939; 2944; 2946; 2947; 2948; 2949; 2950; 2952; 2953; 2956; 2957; 2958; 2965; 2969; 2972; 2990; 3002; 3014; 3020; 3035; 3053;
3064; 3065; 3067; 3075; 3090; 3091; 3093; 3131; 3146; 3148; 3162; 3163; 3172; 3178; 3183; 3184; 3192; 3202; 3265; 3273; 3276; 3291; 3297; 3298; 3301; 3303; 3304;
3305; 3308; 3309; 3312; 3313; 3315; 3320; 3326; 3337; 3339; 3351; 3371; 3375; 3383; 3385; 3428; 3439; 3455; 3456; 3458; 3460; 3479; 3480; 3481; 3482; 3485; 3486;
3488; 3491; 3507; 3512; 3516; 3552; 3553; 3562; 3565; 3569; 3570; 3572; 3576; 3578; 3586; 3596; 3620; 3621; 3630; 3635; 3643; 3655; 3659; 3664; 3667; 3673; 3674;
3675; 3676; 3678; 3684; 3685; 3687; 3688; 3689; 3690; 3691; 3692; 3693; 3699; 3702; 3713; 3716; 3717; 3725; 3777; 3791; 3815; 3817; 3827; 3837; 3845; 3848; 3852;
3855; 3856; 3859; 3861; 3880; 3911; 3921; 3931; 3932; 3933; 3934; 3937; 3952; 3953; 3973; 4000; 4035; 4036; 4057; 4058; 4063; 4068; 4072; 4074; 4082; 4085; 4087;
4088; 4089; 4100; 4125; 4130; 4133; 4134; 4135; 4137; 4152; 4155; 4162; 4170; 4179; 4193; 4194; 4212; 4214; 4216; 4217; 4233; 4257; 4261; 4288; 4292; 4294; 4296;
4297; 4312; 4313; 4314; 4316; 4318; 4331; 4353; 4361; 4436; 4478; 4481; 4485; 4486; 4487; 4496; 4502; 4507; 4524; 4547; 4582; 4583; 4599; 4602; 4609; 4610; 4613;
4629; 4641; 4658; 4666; 4683; 4687; 4690; 4691; 4692; 4751; 4771; 4780; 4790; 4791; 4800; 4801; 4802; 4803; 4811; 4828; 4830; 4835; 4839; 4842; 4843; 4846; 4851;
4867; 4869; 4892; 4904; 4913; 4914; 4915; 4928; 4931; 4943; 4946; 4947; 4950; 4951; 4952; 4968; 4973; 5004; 5027; 5037; 5045; 5047; 5054; 5058; 5062; 5071; 5092;
5104; 5111; 5129; 5144; 5148; 5154; 5155; 5156; 5159; 5174; 5178; 5201; 5216; 5217; 5240; 5243; 5244; 5245; 5265; 5268; 5287; 5295; 5296; 5300; 5310; 5325; 5327;
5328; 5329; 5335; 5336; 5337; 5338; 5339; 5340; 5345; 5360; 5366; 5371; 5378; 5395; 5409; 5411; 5422; 5423; 5430; 5443; 5444; 5451; 5468; 5469; 5478; 5479; 5494;
5515; 5536; 5552; 5562; 5566; 5573; 5576; 5578; 5579; 5580; 5581; 5591; 5592; 5594; 5595; 5596; 5599; 5601; 5602; 5604; 5605; 5606; 5608; 5609; 5610; 5612; 5624;
5653; 5657; 5663; 5664; 5672; 5716; 5727; 5728; 5743; 5747; 5764; 5770; 5771; 5777; 5781; 5782; 5786; 5788; 5791; 5792; 5797; 5805; 5817; 5819; 5830; 5858; 5860;
5868; 5873; 5874; 5879; 5880; 5886; 5887; 5888; 5894; 5898; 5899; 5915; 5918; 5921; 5925; 5928; 5931; 5934; 5950; 5962; 5970; 5976; 5979; 5982; 5983; 5984; 5985;
6052; 6053; 6054; 6055; 6056; 6083; 6093; 6095; 6125; 6135; 6154; 6159; 6176; 6188; 6194; 6195; 6196; 6197; 6198; 6199; 6202; 6206; 6207; 6234; 6240; 6241; 6271;
6273; 6275; 6277; 6278; 6281; 6282; 6285; 6286; 6300; 6317; 6342; 6347; 6352; 6364; 6383; 6386; 6401; 6402; 6403; 6404; 6416; 6442; 6452; 6461; 6464; 6523; 6528;
6531; 6533; 6548; 6550; 6565; 6583; 6590; 6597; 6598; 6606; 6625; 6640; 6647; 6648; 6654; 6655; 6662; 6667; 6670; 6678; 6693; 6696; 6709; 6714; 6721; 6722; 6736;
6750; 6753; 6772; 6774; 6789; 6790; 6794; 6804; 6840; 6850; 6853; 6854; 6872; 6880; 6890; 6897; 6902; 6903; 6904; 6905; 6907; 6908; 6927; 6929; 6950; 7010; 7011;
7012; 7015; 7018; 7019; 7020; 7022; 7027; 7038; 7039; 7040; 7041; 7045; 7046; 7048; 7052; 7056; 7057; 7067; 7068; 7076; 7082; 7100; 7114; 7124; 7132; 7133; 7135;
7150; 7153; 7155; 7156; 7157; 7158; 7159; 7161; 7169; 7170; 7173; 7177; 7184; 7187; 7216; 7248; 7249; 7251; 7252; 7265; 7276; 7277; 7278; 7280; 7283; 7291; 7295;
7296; 7298; 7301; 7302; 7319; 7322; 7323; 7329; 7337; 7341; 7350; 7356; 7399; 7409; 7410; 7412; 7415; 7422; 7428; 7430; 7431; 7436; 7443; 7447; 7448; 7450; 7454;
7456; 7462; 7464; 7486; 7490; 7508; 7515; 7528; 7529; 7531; 7532; 7533; 7534; 7535; 7536; 7707; 7804; 7827; 7832; 7846; 7874; 7982; 8029; 8061; 8178; 8224; 8290;
8295; 8312; 8317; 8339; 8349; 8379; 8411; 8431; 8439; 8451; 8471; 8473; 8516; 8551; 8552; 8554; 8555; 8556; 8557; 8574; 8605; 8607; 8626; 8631; 8647; 8649; 8655;
8659; 8660; 8714; 8717; 8728; 8733; 8743; 8772; 8794; 8795; 8805; 8812; 8837; 8850; 8870; 8871; 8900; 8905; 8906; 8936; 8940; 8944; 8974; 8976; 9013; 9014; 9015;
9022; 9052; 9055; 9093; 9101; 9112; 9131; 9154; 9185; 9191; 9200; 9208; 9219; 9220; 9232; 9261; 9314; 9344; 9349; 9351; 9365; 9368; 9370; 9401; 9402; 9423; 9429;
9457; 9500; 9507; 9510; 9518; 9531; 9540; 9545; 9563; 9564; 9601; 9611; 9622; 9635; 9649; 9656; 9672; 9712; 9722; 9804; 9846; 9868; 9910; 9961; 10009; 10018; 10019;
10024; 10026; 10049; 10055; 10057; 10075; 10095; 10096; 10097; 10111; 10121; 10146; 10153; 10155; 10163; 10188; 10210; 10221; 10252; 10253; 10254; 10257; 10273;
10274; 10376; 10382; 10383; 10397; 10399; 10417; 10434; 10452; 10461; 10474; 10482; 10498; 10524; 10529; 10531; 10544; 10576; 10599; 10657; 10694; 10728; 10733;
10769; 10817; 10818; 10825; 10848; 10856; 10891; 10928; 10953; 10957; 10963; 10987; 11010; 11096; 11144; 11183; 11184; 11186; 11200; 11252; 11276; 11309; 22795;
22821; 22895; 22948; 22974; 22976; 23022; 23048; 23049; 23059; 23062; 23071; 23113; 23162; 23305; 23368; 23411; 23491; 23513; 23559; 23562; 23621; 23640; 23645;
25898; 25932; 25942; 26085; 26088; 26191; 26257; 26287; 26578; 26986; 27036; 27040; 27085; 27113; 27130; 27240; 27250; 27343; 28231; 28232; 28234; 28996; 29108;
29124; 29760; 29899; 29904; 29979; 30001; 30008; 30011; 30837; 50484; 50486; 51053; 51138; 51143; 51147; 51206; 51246; 51365; 51512; 51517; 51555; 51604; 51703;
51726; 51741; 51754; 53358; 54812; 54822; 54900; 54971; 55103; 55207; 55243; 55333; 55503; 55651; 55800; 55856; 55914; 55929; 56302; 56605; 57085; 57147; 57172; 57504; 57761; 58480; 58484; 64151;
64215; 64326; 64386; 64714; 64756; 64805; 65981; 79023; 79038; 79634; 79796; 80205; 80306; 80854; 81572; 81928; 81929; 83874; 83931; 84168; 84289; 84623; 84699; 84962; 85415; 90070; 91419; 91647;
91768; 94005; 94025; 94241; 114655; 121504; 122876; 127829; 129401; 132250; 137735; 143425; 149428; 153090; 157310; 162989; 170589; 171017; 171024; 171221; 192343; 201161; 202559; 203068; 204851;
221357; 246181; 246182; 259215; 283208; 344148; 347733; 348995; 391538; 405754; 440533; 654443
chimpanzee subset 1 (mp)
468329; 452266; 457782; 461767; 455414; 473385; 453526; 451999; 470228; 454891; 461258; 461260; 453745; 461560; 453828; 470195; 460228; 459027; 453001;
471709; 453133; 464075; 465606; 457698; 461953; 461631; 457777; 457428; 469197; 468459; 454876; 463949; 471960; 457427; 466438; 465696; 452979; 454879;
454882; 455312; 454877; 454878; 468330; 454890; 461559; 456755; 454883; 457094; 463949; 460437; 453487; 463444; 460561; 450803; 466704; 462010; 465602;
457025; 457891; 461780; 457306; 450133; 472094; 462344; 464617
chimpanzee subset 2 (mp)
453307; 468459; 458633; 450170; 457688; 463707; 464075; 450128; 457428; 458589; 454179; 463993; 467413; 453867; 468637; 465938; 454636; 457500; 462304;
450194; 457457; 456208; 461558; 461560; 454486; 450178; 458868; 467220; 459670; 457128; 463443; 457782; 457602; 457867; 466302; 460914; 464733; 468747;
450196; 450200; 470203; 469657; 459742; 454730; 468295; 458200; 456911; 461315; 461316; 469100; 466983; 449638; 456919; 452006; 459713; 463671; 453914;
465979; 464003; 460377; 467139; 466205; 465851; 465448; 452318; 464845; 451439; 453132; 458081; 461860; 454962; 458844; 462188; 462112; 454456; 455205;
457657; 461836; 455855; 454340; 465264; 472748; 470996; 454454; 452875; 465543; 463547; 450503; 454845; 459203; 459155; 458076; 465803; 464422; 460522;
463759; 469970; 467135; 463494; 457607; 450865; 451812; 463253; 460182; 461762; 450617; 450415; 461020; 463127; 457320; 457306; 468403; 461080; 457351;
458779; 459171; 452897; 450101; 458229; 462709; 465606; 465028; 464407; 456060; 472992; 460243; 453319; 466934; 457968; 467668; 465078; 458000; 453178;
451065; 466436; 450779; 467865; 453269; 459882; 457738; 455647; 464967; 472732; 454409; 458778; 472830; 460651; 453907; 471965; 450818; 461517; 473846;
450323; 467715; 465251; 468432; 461399; 454154; 457354; 472713; 463762; 460219; 463094; 462935; 463688; 454279; 459022; 454750; 453428; 453217; 465855;
457448; 451255; 462238; 454865; 464469; 459208; 470057; 464116; 462797; 454246; 471959; 466011; 457463; 470872; 452245; 455138; 463847; 456159; 465650;
463568; 459156; 456675; 460954; 461476; 453803; 462344; 457076; 459492; 460154; 452070; 456705; 458498; 472005; 467064; 462168; 451286; 466759; 460392;
464368; 452684; 461721; 467937; 467890; 463192; 453825; 464451; 456191; 461248; 469226; 453759; 457136; 450655; 460431; 464532; 468976; 450735; 469081; 464331; 464550; 451335; 465527; 472832;
456429; 460345; 470894; 460351; 460911; 454258; 462739; 460744; 467923; 459277; 453429; 464081; 460137; 458393; 453003; 471527; 469720; 457954; 457502; 459183; 456123; 450344; 457541; 456558;
451157; 461133; 458749; 464321; 459495; 460085; 465382; 459678; 451963; 454483; 455335; 456041; 468014; 472838; 473837; 466425; 457281; 464049; 457964; 454111
chimpanzee subset 3 (mp)
464023; 460785; 464802; 459914; 454895; 466148; 450133; 453307; 454959; 461396; 458714; 455009; 457785; 471690; 451363; 454228; 461258; 460762; 453202;
456035; 464675; 460416; 461260; 464623; 459126; 455242; 455103; 463363; 472953; 453487; 461466; 462198; 452153; 461999; 457427; 465200; 458013; 459058;
468459; 466149; 469584; 465680; 451869; 462999; 459054; 454450; 464022; 468172; 455397; 451999; 458487; 471061; 458633; 460745; 456057; 450170; 458500;
451291; 465041; 462616; 470638; 460830; 458156; 458635; 453650; 463366; 464069; 470705; 449497; 463781; 452526; 455644; 457688; 464703; 449612; 461902;
456886; 456887; 473223; 459213; 455756; 463707; 462330; 451317; 451519; 450745; 460110; 456457; 451116; 463670; 451690; 460566; 461467; 461835; 455914;
463421; 451775; 470617; 457154; 465075; 462191; 456066; 450481; 468234; 452025; 463891; 463534; 465021; 456062; 455931; 469969; 467982; 466666; 463674;
462868; 469725; 450673; 464075; 450128; 463542; 450508; 450717; 457690; 467891; 470146; 457428; 458589; 462643; 462187; 471627; 458975; 456756; 454774;
462182; 454989; 458829; 461766; 452089; 463003; 454179; 463993; 457677; 467413; 457278; 472471; 468068; 472893; 453745; 456052; 450909; 450626; 470228;
450857; 463579; 461905; 460372; 462613; 455426; 459332; 451165; 456929; 452017; 468012; 460968; 464766; 453867; 455696; 451553; 470885; 455352; 462420;
471248; 471742; 458184; 454356; 462097; 461391; 460888; 468637; 455668; 465938; 458862; 457777; 464074; 455728; 454636; 451980; 456124; 470526; 463079;
460906; 451158; 457038; 457500; 452679; 462304; 462413; 465257; 459974; 464073; 469538; 450194; 457457; 456208; 461559; 461558; 461067; 461560; 470835;
454486; 452508; 462339; 453047; 470371; 468262; 450178; 458868; 461516; 467678; 460599; 451783; 454741; 471560; 467220; 465866; 462776; 469986; 459670;
455228; 463410; 459695; 471681; 470895; 462769; 457128; 457094; 451717; 458962; 459218; 467523; 453483; 451061; 463472; 463443; 466864; 457782; 462122;
464259; 453425; 457602; 452958; 461178; 455140; 452518; 461606; 470195; 453880; 458269; 461216; 457867; 463310; 466302; 460914; 468001; 462974; 471967;
462060; 464733; 451629; 462099; 463488; 468747; 456606; 462833; 450196; 457424; 473197; 458622; 473183; 449517; 470077; 453676; 466318; 463112; 459932;
463396; 459933; 456988; 461276; 461152; 470485; 450200; 471626; 449565; 463288; 469506; 461866; 471205; 469657; 464134; 450954; 455649; 459742; 457701;
454730; 455111; 470596; 451958; 459807; 454070; 450396; 458607; 468295; 458200; 460436; 456911; 456893; 470334; 461315; 461316; 469100; 460916; 473387;
466983; 468241; 454664; 464914; 464764; 471734; 449638; 456919; 457385; 452006; 459713; 460325; 453350; 457438; 459214; 461661; 455407; 468537; 453380;
451428; 455418; 457274; 469663; 457658; 471543; 463119; 463671; 473380; 453914; 450866; 471095; 451587; 451510; 454094; 451511; 451509; 452954; 451488;
459215; 464003; 460377; 461092; 471221; 471397; 458565; 463016; 459043; 452434; 464278; 453273; 459769; 461406; 452191; 456799; 455134; 462395; 451784;
467139; 455026; 459483; 462261; 466205; 470311; 450971; 457771; 465851; 460152; 473362; 465448; 452318; 453652; 464845; 472467; 451439; 460965; 466103;
453132; 458081; 457669; 461860; 454962; 458844; 461318; 462188; 457979; 469030; 462112; 454456; 455205; 467541; 468566; 457657; 461836; 455855; 454340;
472748; 450103; 454268; 470996; 454454; 458291; 453736; 459827; 452875; 465543; 459074; 463547; 460178; 454629; 452950; 471633; 456150; 450503; 471564;
454845; 459203; 464165; 454037; 468536; 450445; 453526; 454515; 468310; 459155; 459593; 467503; 457790; 465803; 465254; 466142; 464422; 463759; 469970;
455504; 467135; 463494; 457607; 450865; 456828; 456112; 451812; 465371; 453455; 468542; 463253; 473012; 453339; 460182; 470523; 461762; 452715; 456719;
465518; 467978; 450617; 466776; 466665; 456373; 450415; 463467; 452533; 460918; 455326; 457025; 455092; 461020; 453545; 471846; 463127; 450110; 463008;
451051; 459024; 457325; 457321; 457320; 469501; 457306; 457994; 455459; 456862; 459991; 464298; 464192; 450191; 452212; 455065; 468403; 461080; 457351;
458779; 468767; 460196; 460627; 458702; 449634; 459171; 452897; 450101; 451939; 459750; 471709; 467939; 461366; 458229; 462709; 449510; 471035; 458807;
470721; 458275; 469980; 468641; 463463; 450383; 465606; 465701; 461951; 465560; 462699; 455000; 469714; 468650; 463368; 465028; 471443; 460710; 466079;
470954; 464407; 470399; 456060; 467962; 462081; 472992; 460243; 469898; 453319; 453698; 457762; 466934; 457968; 469636; 472883; 460232; 454488; 467668;
465078; 458000; 470287; 452187; 453178; 451065; 470654; 472356; 450779; 467112; 453352; 462108; 467865; 453269; 459882; 461504; 466634; 457738; 455647;
464967; 453148; 472732; 460195; 456094; 454409; 458778; 472830; 459427; 466656; 460651; 469648; 451875; 453907; 463672; 463564; 469380; 471883; 469477;
467212; 464193; 452694; 473846; 465702; 450323; 467715; 465251; 454633; 469196; 468432; 461399; 456575; 455128; 454154; 472892; 460852; 451389; 472713;
463762; 459872; 460219; 462547; 463094; 467260; 467407; 462935; 463688; 454279; 459022; 464921; 473369; 453656; 453879; 453428; 453217; 465855; 457448;
450331; 460050; 451255; 462238; 464640; 454619; 454865; 471032; 464469; 455083; 471251; 472076; 465641; 457453; 470057; 468770; 468721; 454246; 468370;
469101; 471959; 466011; 457463; 470872; 465831; 459487; 452245; 451878; 456962; 463847; 456159; 460878; 465650; 463568; 462054; 466191; 471711; 459156;
456353; 456675; 460954; 461476; 459661; 453803; 460717; 468685; 462344; 459492; 460154; 451267; 451329; 450267; 458198; 452070; 465331; 456705; 458498;
461480; 472005; 467064; 468949; 461140; 458274; 451286; 466759; 467075; 458831; 467451; 460392; 458733; 470231; 454606; 466711; 452684; 461721; 458157; 467937; 467890; 453987; 464616; 463192;
453825; 462044; 453193; 466088; 464451; 470085; 451573; 456191; 461248; 469226; 453759; 457136; 467600; 450655; 464532; 468976; 464361; 473146; 450735; 458390; 453658; 454067; 469081; 454350;
464550; 451335; 465527; 472832; 457697; 462477; 456429; 460345; 470894; 458984; 460351; 471010; 460911; 454258; 459277; 453429; 457549; 460137; 453003; 462318; 466810; 471527; 469720; 457954;
457502; 469667; 459183; 469852; 459135; 471153; 450344; 457541; 456558; 465366; 452180; 453192; 459753; 451542; 461133; 468602; 458749; 460085; 468672; 465382; 451963; 452031; 454483; 455335;
455029; 467477; 459802; 472838; 466425; 457281; 464049; 461456; 455013; 471960; 457964; 460093; 471856

human subset 1 (i)

| | | |
|---|---|---|
| 1555 | CYP2B6 | cytochrome P450, family 2, subfamily B, polypeptide 6 |
| 4616 | GADD45B | growth arrest and DNA-damage-inducible, beta |
| 8848 | TSC22D1 | TSC22 domain family, member 1 |
| 84445 | LZTS2 | leucine zipper, putative tumor suppressor 2 |
| 572 | BAD | BCL2-antagonist of cell death |
| 23764 | MAFF | v-maf musculoaponeurotic fibrosarcoma oncogene homolog F (avian) |
| 3479 | IGF1 | insulin-like growth factor 1 (somatomedin C) |
| 3481 | IGF2 | insulin-like growth factor 2 (somatomedin A) |
| 3484 | IGFBP1 | insulin-like growth factor binding protein 1 |
| 3488 | IGFBP5 | insulin-like growth factor binding protein 5 |
| 9489 | PGS1 | phosphatidylglycerophosphate synthase 1 |
| 1026 | CDKN1A | cyclin-dependent kinase inhibitor 1A (p21, Cip1) |
| 900 | CCNG1 | cyclin G1 |
| 4616 | GADD45B | growth arrest and DNA-damage-inducible, beta |
| 1958 | EGR1 | early growth response 1 |
| 2272 | FHIT | fragile histidine triad gene |
| 51741 | WWOX | WW domain containing oxidoreductase |
| 1956 | EGFR | epidermal growth factor receptor (erythroblastic leukemia viral (v-erb-b) oncogene homolog, avian) |
| 3624 | INHBA | inhibin, beta A (activin A, activin AB alpha polypeptide) |
| 3726 | JUNB | jun B proto-oncogene |
| 1154 | CISH | cytokine inducible SH2-containing protein |

human subset 2 (i)
596; 597; 598; 599; 1020; 1027; 1029; 1387; 1457; 1460; 1647; 1655; 2033; 2057; 2059; 2060; 2064; 2065; 2069; 2099; 2185; 2241; 2308; 2309; 2353; 2547; 2549; 2580;
2771; 2885; 2886; 2887; 2888; 3064; 3065; 3084; 3091; 3320; 3371; 3480; 3483; 3484; 3485; 3486; 3487; 3488; 3489; 3490; 3547; 3560; 3563; 3625; 3626; 3636; 3643;
3678; 3725; 3732; 3855; 3856; 3872; 3875; 4088; 4089; 4170; 4193; 4286; 4303; 4582; 4609; 4616; 4664; 4665; 4690; 4773; 4779; 4856; 5058; 5111; 5159; 5287; 5292;
5295; 5296; 5306; 5307; 5335; 5338; 5339; 5359; 5425; 5465; 5467; 5468; 5469; 5499; 5524; 5566; 5573; 5578; 5599; 5684; 5747; 5753; 5770; 5777; 5781; 5795; 5921;
5925; 5970; 6004; 6281; 6418; 6464; 6597; 6622; 6633; 6642; 6643; 6654; 6655; 6667; 6696; 6714; 6772; 6774; 6776; 6777; 7018; 7039; 7049; 7057; 7082; 7083; 7157;
7161; 7251; 7329; 7409; 7410; 7448; 7529; 7531; 7533; 7534; 7709; 8038; 8061; 8317; 8440; 8560; 8649; 8651; 8723; 8737; 8882; 8900; 9020; 9021; 9046; 9063; 9412;
10017; 10045; 10188; 10272; 10451; 10538; 10912; 10971; 11040; 11184; 23214; 23624; 25792; 25884; 53358; 56647; 58533; 81628; 83481; 90480; 121504; 122953;
255488
human subset 3 (i)
2; 25; 58; 90; 91; 92; 93; 94; 102; 142; 154; 156; 160; 161; 163; 185; 207; 268; 308; 317; 328; 332; 355; 356; 367; 374; 378; 387; 388; 466; 467; 472; 572; 578; 581; 595;
596; 597; 598; 599; 604; 633; 637; 638; 639; 650; 655; 657; 660; 664; 665; 672; 673; 685; 800; 801; 805; 808; 815; 816; 818; 819; 834; 836; 846; 847; 857; 859; 860; 864;
867; 868; 887; 891; 892; 896; 898; 901; 929; 949; 958; 959; 960; 961; 983; 993; 999; 1000; 1017; 1019; 1020; 1021; 1025; 1026; 1027; 1028; 1029; 1030; 1031; 1032; 1033;
1045; 1050; 1051; 1113; 1191; 1232; 1277; 1280; 1281; 1387; 1398; 1401; 1432; 1435; 1445; 1457; 1460; 1490; 1491; 1499; 1508; 1543; 1546; 1554; 1556; 1558; 1559;
1562; 1571; 1576; 1580; 1634; 1636; 1647; 1655; 1745; 1796; 1839; 1869; 1890; 1950; 1956; 1958; 1959; 1960; 1961; 1978; 1993; 1994; 2002; 2033; 2035; 2056; 2057;
2059; 2060; 2064; 2065; 2066; 2067; 2069; 2078; 2099; 2100; 2147; 2149; 2150; 2152; 2155; 2185; 2237; 2241; 2247; 2252; 2260; 2261; 2263; 2272; 2274; 2308; 2335;
2353; 2354; 2355; 2475; 2512; 2547; 2549; 2580; 2660; 2672; 2688; 2690; 2719; 2720; 2771; 2810; 2833; 2852; 2885; 2886; 2887; 2888; 2889; 2890; 2922; 2925; 2932;
3064; 3065; 3066; 3082; 3084; 3091; 3162; 3190; 3206; 3265; 3269; 3274; 3320; 3371; 3397; 3416; 3429; 3458; 3479; 3480; 3481; 3482; 3483; 3484; 3485; 3486; 3487;
3488; 3489; 3490; 3547; 3553; 3554; 3560; 3567; 3569; 3572; 3576; 3577; 3579; 3623; 3625; 3626; 3630; 3636; 3643; 3659; 3667; 3678; 3685; 3688; 3716; 3717; 3725;
3727; 3732; 3816; 3837; 3845; 3897; 3918; 3932; 3949; 3952; 3953; 3964; 4067; 4088; 4089; 4097; 4129; 4137; 4152; 4170; 4193; 4216; 4233; 4292; 4313; 4314; 4316;
4318; 4322; 4327; 4436; 4502; 4582; 4602; 4605; 4609; 4610; 4616; 4654; 4656; 4664; 4665; 4690; 4734; 4763; 4773; 4778; 4779; 4780; 4790; 4801; 4817; 4839; 4843;
4853; 4856; 4899; 4904; 4914; 4915; 5021; 5029; 5047; 5058; 5069; 5077; 5093; 5111; 5156; 5159; 5170; 5243; 5287; 5292; 5295; 5296; 5300; 5306; 5307; 5324; 5328;
5329; 5335; 5338; 5340; 5347; 5359; 5467; 5468; 5499; 5524; 5536; 5540; 5562; 5563; 5566; 5573; 5578; 5580; 5581; 5583; 5591; 5594; 5595; 5597; 5599; 5604; 5605;
5606; 5618; 5657; 5684; 5728; 5731; 5734; 5737; 5743; 5747; 5753; 5770; 5777; 5781; 5788; 5795; 5796; 5879; 5894; 5915; 5921; 5925; 5970; 5981; 5997; 6004; 6012;
6195; 6196; 6275; 6297; 6387; 6418; 6446; 6456; 6462; 6464; 6597; 6598; 6622; 6642; 6654; 6655; 6667; 6670; 6678; 6696; 6714; 6756; 6760; 6772; 6774; 6776; 6777;
6794; 6850; 6868; 6929; 7015; 7039; 7040; 7046; 7049; 7056; 7057; 7082; 7083; 7097; 7124; 7153; 7157; 7161; 7223; 7251; 7291; 7329; 7352; 7409; 7410; 7421; 7422;
7428; 7430; 7448; 7515; 7525; 7529; 7531; 7534; 7709; 7841; 7879; 8027; 8038; 8200; 8202; 8290; 8315; 8412; 8440; 8467; 8503; 8560; 8626; 8649; 8651; 8660; 8737;
8795; 8797; 8839; 8841; 8856; 8878; 8882; 8900; 9020; 9021; 9046; 9134; 9146; 9401; 9518; 9522; 9536; 9564; 9655; 9712; 9836; 9891; 9970; 10006; 10017; 10018; 10044; 10045; 10067; 10135; 10188; 10253;
10254; 10272; 10325; 10399; 10401; 10413; 10468; 10538; 10642; 10657; 10658; 10664; 10818; 10855; 10891; 10912; 10919; 11040; 11177; 11186; 23426; 23624; 25759; 25792; 25884; 26018; 26512; 26574;
27063; 27113; 27250; 29117; 30011; 51214; 51738; 51741; 53358; 54206; 56547; 56647; 57099; 60495; 60676; 63970; 64975; 81628; 90480; 116092; 121504; 122809; 122953; 140885; 152189; 171017; 255488;
283120; 286343; 442219; 492304
chimpanzee subset 1 (i)
456052; 452573; 466256; 451291; 466318; 463395; 459933; 462243; 462097; 470835; 454258; 463371; 468739; 470819
chimpanzee subset 2 (i)
458156; 452796; 463891; 465021; 467891; 456929; 458862; 455728; 473379; 456850; 454636; 451980; 463079; 464073; 461981; 453047; 458868; 461516; 461048;
463410; 459695; 464259; 452958; 453676; 463395; 459932; 463396; 454652; 459933; 451929; 465861; 470203; 470524; 467043; 451403; 455649; 451958; 456893;
466498; 466983; 468261; 468537; 457274; 473659; 459832; 469972; 455185; 451439; 458081; 462188; 457657; 462656; 461836; 455855; 462072; 454454; 460757;
472654; 460178; 454629; 464784; 454845; 450445; 464422; 469970; 467135; 451181; 461762; 452715; 466665; 469608; 457306; 457351; 461375; 456134; 462022;
459171; 452897; 451939; 461366; 458229; 470721; 454681; 454679; 460710; 470399; 457016; 453319; 453698; 468342; 451065; 467865; 455647; 454409; 458778;
472830; 450818; 469380; 459472; 457770; 461399; 460652; 467260; 454750; 464036; 455403; 465356; 467685; 468688; 460954; 455531; 457076; 464585; 470302;
473600; 464902; 466898; 450814; 463600; 453048
chimpanzee subset 3 (i)
464802; 470565; 451911; 459646; 451910; 453472; 457785; 471690; 451363; 468956; 460762; 453202; 455569; 461466; 452153; 465200; 466149; 469584; 465680;
461251; 464506; 459054; 452429; 451291; 462616; 458156; 452796; 473829; 458635; 470232; 462904; 458090; 450572; 450831; 464069; 449497; 463781; 461250;
453100; 459213; 456153; 451519; 451116; 463670; 472017; 451690; 460566; 466414; 461835; 462689; 455914; 461229; 462118; 452359; 473802; 460569; 450481;
460341; 468509; 452025; 463891; 463534; 465021; 455931; 469969; 464075; 470810; 459815; 467891; 457428; 462643; 457127; 463003; 456939; 463993; 450909;
450626; 450857; 452120; 454782; 456929; 459740; 458184; 458941; 462097; 464048; 465023; 455668; 473591; 458862; 456689; 463609; 455728; 473379; 456850;
454636; 451980; 459919; 458548; 463079; 451158; 471510; 461800; 457038; 464073; 451242; 461981; 461067; 470835; 453047; 456127; 459115; 458868; 461516;
461048; 471560; 465866; 460254; 463410; 459695; 471714; 468551; 465514; 470895; 464259; 462950; 463503; 452958; 470195; 464554; 466302; 470746; 471750;
469912; 450608; 453250; 449517; 453676; 466318; 463112; 463395; 459932; 463396; 454652; 459933; 451929; 465861; 450200; 459460; 470203; 463288; 461866;
471205; 470645; 470644; 470524; 467043; 450954; 451403; 455649; 451958; 459807; 450396; 456911; 456893; 466498; 468971; 473387; 457571; 455722; 449638;
456919; 469155; 472771; 468537; 468359; 465588; 455418; 457274; 463119; 463671; 471095; 454094; 451511; 451509; 451513; 459215; 471221; 463016; 458262;
451057; 459832; 467689; 454566; 469972; 455185; 459769; 461406; 452191; 451784; 455026; 472513; 470311; 466702; 464845; 451439; 464690; 459300; 458081;
461318; 462188; 457657; 462656; 461836; 455855; 462072; 464185; 450103; 454454; 453991; 460757; 460178; 464784; 456150; 466064; 471564; 454845; 459203;
464165; 454037; 450445; 453526; 454515; 466142; 450195; 456970; 464422; 469970; 467135; 457607; 451181; 463253; 460182; 461762; 452715; 466665; 461174;
469623; 469608; 463127; 457320; 463011; 465008; 455215; 457351; 458702; 461375; 459171; 452897; 451939; 459750; 471709; 461366; 458229; 473615; 455345;
470721; 454681; 454679; 468641; 459018; 468650; 471443; 470399; 456060; 472992; 457016; 469898; 453319; 453698; 468342; 471325; 452544; 451065; 467865;
455647; 451858; 456094; 455322; 454409; 472830; 470919; 450818; 458308; 457032; 459472; 461517; 456565; 457770; 461399; 458271; 467260; 454750; 464036; 464292; 454966; 464469; 468770; 461788;
473071; 454246; 459209; 466011; 458014; 467685; 459487; 464753; 468688; 457363; 463648; 460954; 459661; 455531; 462344; 457223; 451506; 461879; 456705; 451173; 467064; 461206; 461140; 464585;
462572; 473600; 452855; 460392; 464902; 466898; 460490; 452729; 454601; 450735; 465162; 465527; 470744; 454258; 469787; 450977; 450814; 453297; 457543; 463600; 469955; 452919; 453048; 458039;
460249; 462463; 472042

human subset 1 (l)

| | | |
|---|---|---|
| 5127 | PCTK1 | PCTAIRE protein kinase 1 |
| 1510 | CTSE | cathepsin E |
| 2958 | GTF2A2 | general transcription factor IIA, 2, 12 kDa |
| 1513 | CTSK | cathepsin K (pycnodysostosis) |
| 7534 | YWHAZ | tyrosine 3-monooxygenase/tryptophan 5-monooxygenase activation protein, zeta polypeptide |
| 7142 | TNP2 | transition protein 2 (during histone to protamine replacement) |
| 89777 | SERPINB12 | serpin peptidase inhibitor, clade B (ovalbumin), member 12 |
| 7257 | TSNAX | translin-associated factor X |
| 5020 | OXT | oxytocin, prepro-(neurophysin I) |
| 3939 | LDHA | lactate dehydrogenase A |
| 5334 | PLCL1 | phospholipase C-like 1 |
| 50486 | G0S2 | G0/G1switch 2 |
| 4942 | OAT | ornithine aminotransferase (gyrate atrophy) |
| 1544 | CYP1A2 | cytochrome P450, family 1, subfamily A, polypeptide 2 |
| 1891 | ECH1 | enoyl Coenzyme A hydratase 1, peroxisomal |
| 26061 | PHYH2 | phytanoyl-CoA 2-hydroxylase 2 |
| 1776 | DNASE1L3 | deoxyribonuclease I-like 3 |
| 3158 | HMGCS2 | 3-hydroxy-3-methylglutaryl-Coenzyme A synthase 2 (mitochondrial) |
| 1581 | CYP7A1 | cytochrome P450, family 7, subfamily A, polypeptide 1 |
| 1831 | TSC22D3 | TSC22 domain family, member 3 |
| 5601 | MAPK9 | mitogen-activated protein kinase 9 |
| 10280 | OPRS1 | opioid receptor, sigma 1 |
| 6822 | SULT2A1 | sulfotransferase family, cytosolic, 2A, dehydroepiandrosterone (DHEA)-preferring, member 1 |
| 55365 | HCA112 | hepatocellular carcinoma-associated antigen 112 |
| 1544 | CYP1A2 | cytochrome P450, family 1, subfamily A, polypeptide 2 |
| 6447 | SCG5 | secretogranin V (7B2 protein) |
| 4830 | NME1 | non-metastatic cells 1, protein (NM23A) expressed in |
| 488 | ATP2A2 | ATPase, Ca++ transporting, cardiac muscle, slow twitch 2 |
| 2921 | CXCL3 | chemokine (C—X—C motif) ligand 3 |
| 5096 | PCCB | propionyl Coenzyme A carboxylase, beta polypeptide |
| 6446 | SGK | serum/glucocorticoid regulated kinase |
| 2244 | FGB | fibrinogen beta chain |
| 55365 | HCA112 | hepatocellular carcinoma-associated antigen 112 |
| 5734 | PTGER4 | prostaglandin E receptor 4 (subtype EP4) |
| 284541 | CYP4A22 | cytochrome P450, family 4, subfamily A, polypeptide 22 |
| 284541 | CYP4A22 | cytochrome P450, family 4, subfamily A, polypeptide 22 |
| 6581 | SLC22A3 | solute carrier family 22 (extraneuronal monoamine transporter), member 3 |
| 23600 | AMACR | alpha-methylacyl-CoA racemase |
| 3145 | HMBS | hydroxymethylbilane synthase |
| 9807 | IHPK1 | inositol hexaphosphate kinase 1 |
| 7295 | TXN | thioredoxin |
| 3665 | IRF7 | interferon regulatory factor 7 |
| 1581 | CYP7A1 | cytochrome P450, family 7, subfamily A, polypeptide 1 |
| 9153 | SLC28A2 | solute carrier family 28 (sodium-coupled nucleoside transporter), member 2 |
| 4780 | NFE2L2 | nuclear factor (erythroid-derived 2)-like 2 |
| 7832 | BTG2 | BTG family, member 2 |
| 10280 | OPRS1 | opioid receptor, sigma 1 |
| 6822 | SULT2A1 | sulfotransferase family, cytosolic, 2A, dehydroepiandrosterone (DHEA)-preferring, member 1 |
| 5601 | MAPK9 | mitogen-activated protein kinase 9 |

human subset 2 (l)
596; 2266; 2353; 2960; 3297; 3577; 3579; 3725; 4018; 4615; 4790; 4791; 4811; 4832; 5104; 5275; 5350; 5468; 5894; 5925; 5970; 6095; 6096; 6236; 6271; 6317; 6418;
6790; 6874; 6908; 7074; 7132; 7133; 7157; 7186; 7189; 7531; 7532; 7533; 7534; 7975; 8550; 8655; 8660; 8717; 9181; 9261; 9337; 9351; 9451; 9463; 9467; 9519; 9564;
9610; 9759; 9817; 10060; 10142; 10256; 10438; 10628; 10746; 10749; 11016; 11036; 11243; 23162; 23327; 23353; 23764; 25932; 28954; 29883; 50813; 53616; 54434;
54836; 54961; 55815; 58497; 80824; 84817; 85464; 142679; 353376
human subset 3 (l)
2; 15; 60; 100; 150; 151; 152; 183; 207; 287; 328; 408; 468; 487; 551; 571; 572; 596; 613; 673; 694; 811; 815; 834; 836; 844; 845; 867; 890; 891; 941; 960; 993; 994; 995;
998; 999; 1003; 1020; 1027; 1233; 1236; 1277; 1371; 1386; 1387; 1432; 1437; 1439; 1452; 1499; 1509; 1511; 1514; 1519; 1520; 1543; 1545; 1551; 1557; 1558; 1559; 1562;
1565; 1571; 1576; 1577; 1579; 1580; 1586; 1593; 1728; 1759; 1774; 1775; 1906; 1956; 1958; 2004; 2033; 2048; 2064; 2099; 2100; 2153; 2192; 2235; 2243; 2266; 2308;
2309; 2335; 2353; 2494; 2495; 2516; 2597; 2627; 2671; 2697; 2745; 2801; 2810; 2811; 2812; 2908; 2919; 2920; 2932; 2947; 2957; 2960; 2961; 2962; 2963; 3001; 3002;
3010; 3020; 3148; 3150; 3156; 3162; 3169; 3170; 3171; 3172; 3177; 3178; 3184; 3219; 3224; 3276; 3290; 3297; 3383; 3480; 3485; 3553; 3569; 3576; 3581; 3586; 3620;
3630; 3632; 3659; 3660; 3661; 3663; 3664; 3667; 3678; 3688; 3710; 3725; 3726; 3727; 3732; 3752; 3753; 3814; 3827; 3838; 3875; 3940; 3945; 3984; 4018; 4097; 4137;
4193; 4215; 4217; 4303; 4316; 4609; 4613; 4615; 4641; 4654; 4687; 4689; 4734; 4751; 4772; 4773; 4775; 4776; 4778; 4779; 4790; 4791; 4811; 4831; 4832; 4843; 4899;
4943; 5021; 5052; 5095; 5104; 5111; 5126; 5129; 5132; 5163; 5170; 5208; 5243; 5275; 5294; 5345; 5350; 5373; 5443; 5468; 5498; 5550; 5562; 5578; 5587; 5598; 5599;
5600; 5602; 5605; 5629; 5728; 5731; 5732; 5733; 5737; 5743; 5747; 5783; 5894; 5923; 5925; 5970; 5998; 6095; 6096; 6236; 6262; 6271; 6300; 6317; 6337; 6338; 6340;
6416; 6418; 6464; 6507; 6512; 6513; 6523; 6530; 6532; 6571; 6580; 6582; 6667; 6678; 6688; 6783; 6790; 6820; 6869; 6872; 6874; 6880; 6908; 6916; 7014; 7054; 7074;
7100; 7124; 7128; 7141; 7157; 7161; 7166; 7186; 7189; 7247; 7249; 7296; 7350; 7351; 7352; 7389; 7422; 7431; 7529; 7531; 7532; 7533; 7809; 7841; 7975; 8125; 8382;
8452; 8551; 8552; 8600; 8660; 8839; 8850; 9022; 9154; 9181; 9261; 9270; 9337; 9351; 9411; 9420; 9451; 9467; 9479; 9519; 9564; 9759; 9817; 9965; 9971; 9978; 10010; 10018; 10060; 10110; 10140; 10256;
10397; 10438; 10472; 10494; 10512; 10587; 10628; 10746; 10749; 10766; 10783; 10855; 10891; 10923; 10971; 10987; 10991; 11016; 11036; 11037; 11183; 11221; 11243; 23162; 23327; 23678; 23764; 25855;
25932; 27185; 28959; 29883; 30819; 50813; 51284; 51447; 53616; 53841; 54766; 54836; 55450; 56302; 57172; 57447; 58497; 58531; 64283; 64708; 80824; 84817; 117283; 117583; 158222; 283458; 286749
chimpanzee subset 1 (l)
465602; 457677; 453483; 457278; 472830; 467904; 468567; 457819; 470020; 470611; 457697; 450803; 453745; 460204; 460477; 457169; 464191; 465062; 456409;
463949; 463949; 463833; 453745; 453292; 455134; 461255; 460716; 463011; 461558; 463833; 450195; 461953; 460379; 450779; 450927; 464191; 453400; 459769;
469648; 465062; 456409; 463949
chimpanzee subset 2 (l)
461560; 453047; 460615; 470645; 470644; 456893; 463116; 460269; 461406; 453132; 460178; 460182; 452715; 466665; 472956; 457325; 455459; 469980; 466934;
457968; 464876; 450474; 454409; 458778; 472830; 454154; 457378; 462213; 459388; 463010; 454246; 466675; 455707; 472440; 457986; 454459; 467007; 457388;
453825; 455443; 472263; 469226; 458153; 451792; 463517; 473027; 451365; 454174; 465297; 455246; 454551
chimpanzee subset 3 (l)
468336; 450133; 466201; 459410; 450104; 453202; 465200; 468334; 454019; 469863; 458500; 451291; 463781; 452121; 455756; 451519; 451690; 461467; 461835;
470892; 460341; 458064; 456609; 463891; 470781; 454654; 460544; 467891; 462643; 471627; 458975; 462182; 467413; 461567; 457277; 450909; 450626; 470228;
450857; 463579; 468012; 464766; 462437; 462097; 467100; 458862; 456613; 454636; 463079; 457500; 458908; 461559; 461560; 453047; 469631; 451783; 468166;
471681; 470895; 457128; 467523; 460615; 464098; 455639; 461873; 471879; 457782; 461606; 458552; 471516; 470195; 456143; 458269; 451345; 461216; 457698;
450196; 453676; 459932; 450200; 463288; 471205; 469657; 464134; 450954; 461629; 457701; 451958; 450396; 456893; 468739; 466498; 457654; 460916; 454841;
463466; 463116; 468359; 468286; 473659; 451509; 459043; 460269; 451057; 467689; 455485; 469972; 452821; 455185; 461406; 455026; 472513; 456537; 452641;
453132; 458081; 458106; 457669; 469614; 459743; 457687; 463649; 453905; 459074; 460178; 457450; 462903; 471564; 452834; 450445; 457728; 466142; 467459;
456970; 464422; 461359; 460182; 452715; 466665; 464676; 472956; 457325; 455459; 451776; 453984; 455065; 457351; 461918; 469329; 458779; 454096; 466213;
472177; 463113; 451939; 471709; 461284; 469980; 470412; 465701; 463774; 454201; 450955; 457762; 472136; 466453; 464876; 450474; 459586; 467112; 461504; 451416; 456541; 454409; 458778; 462089;
459983; 458271; 460219; 464921; 457378; 462213; 457036; 459388; 451148; 463010; 454246; 455707; 452162; 458860; 459685; 459487; 468406; 469733; 470704; 472427; 458654; 457986; 454459; 458865;
473058; 461206; 461140; 470302; 466759; 467007; 467451; 457388; 453825; 455443; 464217; 469226; 457820; 450692; 451792; 460352; 463517; 451538; 473027; 469667; 461808; 460010; 465297; 455246

TABLE 5B
toxicology biomarkers (non-primate)

dog subset 1 (f)
403435; 404011; 612545; 487382; 403779; 403886; 403461; 488776; 487261; 489673; 475192; 485401; 489541; 480864; 489938; 610139; 608156; 478816; 608911; 480319; 478983; 478438; 612974; 476425;
476288; 403657; 475388; 404013; 476909; 479212; 485407; 479951; 609631; 490207; 474846; 487572; 476825; 485255; 485256; 480784; 485258; 480105; 485208; 403800; 478722; 612906; 610810; 474670;
610745; 476318; 608423; 491235; 608089; 610449; 483200; 480117; 477858; 484385; 474442; 403643; 403643
dog subset 2 (f)
403945; 484472; 479866; 474855; 486077; 404306; 403883; 481105; 403640; 608459; 483312; 403831; 482861; 479668; 481500; 475633; 609832; 479435; 403784; 486731; 611987; 484147; 478868; 477166;
484431; 609956; 482840; 403602; 479483; 403787; 480143; 475851; 487293; 611255; 485864; 485402; 481275; 480621; 478902; 483121; 478295; 479884; 483299; 474733; 607431; 609018; 612823;
478055; 479297
dog subset 3 (f)
477699; 403580; 608134; 490385; 490878; 476322; 476094; 479325; 403945; 403588; 485189; 403669; 449028; 484472; 489378; 491750; 482682; 478194; 487381;
483745; 487746; 487044; 489369; 442858; 481131; 403429; 492302; 487332; 480578; 484791; 484790; 403651; 445450; 475193; 485707; 403512; 608910; 607922;
479866; 474855; 403577; 403856; 489188; 477032; 486077; 608543; 448806; 609214; 487984; 480505; 404306; 611715; 442980; 492129; 491860; 610283; 475356;
403883; 481105; 612282; 403640; 403169; 489287; 486685; 488940; 404015; 476993; 475631; 478927; 481018; 483378; 483680; 608459; 403915; 488808; 480092;
608169; 477726; 490792; 478232; 609642; 475032; 481088; 483808; 403755; 480906; 489224; 403418; 491952; 483312; 482667; 478575; 403831; 476825; 480784;
485256; 485255; 487551; 403441; 482861; 609564; 486068; 479668; 610255; 483664; 491618; 403974; 403989; 403785; 403985; 403850; 403977; 403976; 481500;
475633; 482264; 486148; 609429; 486639; 491042; 478680; 484094; 490978; 403830; 491320; 403616; 484787; 479562; 607693; 481826; 607717; 403438; 488699;
609986; 403768; 485174; 613013; 479435; 480265; 480618; 403402; 479051; 403784; 611764; 474472; 490921; 486731; 611987; 484147; 491866; 403844; 483448;
488544; 477166; 484431; 478001; 480422; 607205; 483266; 609956; 480048; 478207; 482840; 403426; 476446; 485874; 488167; 403602; 611266; 611484; 479483;
490360; 403606; 480805; 490904; 494005; 477575; 486758; 489532; 403832; 403986; 403987; 476915; 489798; 477308; 478667; 477499; 479949; 491768; 491278;
403787; 480143; 475851; 486270; 448790; 449622; 478634; 608210; 479438; 608860; 403751; 403464; 486507; 611255; 608433; 485864; 403475; 488449; 490967;
490969; 475045; 477240; 485402; 611193; 485079; 488213; 403489; 403998; 488596; 485867; 403417; 403922; 403869; 611980; 484641; 486004; 481275; 487509;
478874; 481089; 481590; 403802; 494000; 399544; 476447; 489818; 483900; 404007; 481256; 489997; 485563; 478350; 489684; 610928; 610284; 476241; 478324;
478295; 479884; 483299; 474733; 607431; 476708; 481807; 477283; 475827; 476490; 486638; 403702; 481396; 607899; 609018; 612823; 486989; 479684; 481794;
479127; 477417; 478055; 477133; 488367; 477186; 479297; 490182; 474928; 487314; 482363; 477343; 475189; 478455; 477820; 492140; 482745; 481058; 482744;
612306; 474603; 609299; 490728; 482257; 480761; 483671; 491045; 489540; 491747; 608251
rat subset 1 (f)
rat subset 2 (f)
287774; 81613; 497663; 406169; 64529; 24329; 24337; 29496; 60662; 81504; 24434; 287168; 112400; 24464; 24508; 290749; 24586; 24600; 81737; 25730; 25054; 301430; 25737; 25344; 29741; 25692;
85253; 363633; 24615; 85247; 81778; 140941; 366126; 83805; 25559; 25274; 29169; 363247; 312492; 261736; 116501; 59114; 116645; 25514; 295686; 361319; 303039
rat subset 3 (f)
24153; 81822; 303742; 25591; 24185; 25380; 25673; 79125; 287774; 24208; 25387; 65262; 171374; 58919; 81613; 29363; 25644; 367786; 64022; 298566; 29687; 29153;
50662; 362489; 500985; 25406; 83502; 94201; 83571; 24255; 54241; 57301; 60463; 29393; 24267; 296959; 54242; 24271; 113936; 314855; 81646; 497663; 406169;
60446; 81649; 307403; 64032; 307505; 84353; 64529; 290257; 25699; 303469; 29238; 498003; 24329; 302697; 299606; 302811; 314436; 25043; 60465; 313474; 24337;
29496; 292673; 293701; 24356; 29251; 116677; 24946; 29243; 25288; 114024; 113976; 54250; 116499; 60662; 29348; 84489; 24368; 260321; 314322; 56718; 83826;
25150; 24377; 361388; 24383; 25172; 25453; 24392; 84475; 81504; 29627; 84027; 24434; 25632; 25632; 24440; 361619; 293267; 502359; 94164; 501681; 287168; 24443;
24446; 112400; 29395; 24464; 24482; 24483; 24484; 24494; 116562; 287287; 24498; 84405; 64546; 24508; 290749; 296953; 25467; 24516; 25472; 24917; 361959;
303530; 94106; 170496; 25608; 301034; 311337; 298385; 25684; 361586; 303413; 266714; 288770; 24586; 25490; 24592; 310738; 499194; 24600; 84423; 60355; 81737;
25730; 25054; 302552; 83497; 365410; 24618; 114502; 25509; 25737; 25344; 114209; 24648; 288937; 298947; 29741; 29692; 24653; 29354; 25692; 25619; 25738; 25096;
85253; 287527; 315713; 363633; 171068; 25664; 360774; 24680; 170538; 116590; 50689; 114509; 24691; 50557; 24694; 24695; 362965; 24708; 116468; 288414; 288003;
304528; 81763; 501560; 25271; 24615; 85247; 81778; 288663; 29538; 298006; 24772; 157074; 298596; 363061; 29499; 25550; 84386; 499417; 287993; 24790; 366126;
266602; 83805; 78968; 25124; 25125; 25126; 65189; 311622; 25559; 117526; 171046; 306862; 116592; 24827; 59086; 81809; 56083; 29260; 24835; 24842; 252924;
50659; 84494; 25274; 25232; 301442; 116643; 83785; 24874; 116669; 84495; 56010; 60628; 29448; 310678; 360426; 29359; 296257; 300772; 89842; 29576; 364152;
308265; 363088; 261736; 116501; 83504; 59114; 116645; 140665; 295284; 308508; 25560; 81732; 289820; 305896; 25514; 295686; 83724; 83726; 316213; 83516;
363283; 361319; 362229; 295631; 311546; 303039; 497836; 294392; 362360; 305922; 360915; 85311; 94340; 317589; 114246; 501648; 116469; 294324; 170919; 297432;
81536; 408212; 361686; 303497; 360537; 363463
mouse subset 1 (f)
12306; 52123; 547443; 68943; 18778; 68416; 12262; 19228; 14726; 12064; 67016; 14766; 15900; 109697; 16514; 19377; 56360; 66943; 110033; 94065; 55985; 99031;
13199; 64010; 94346; 66052; 27428; 68734; 56459; 68031; 13645; 230673; 14411; 16648; 52637; 67893; 170823; 73847; 11364; 51793; 21778; 15130; 15132; 15135;
15129; 18366; 109232; 11859; 17748; 109254; 215008; 66451; 625052; 216739; 59003; 107771; 107272; 68929; 227615; 71881; 58184; 76742; 67459; 18242; 68667;
17105; 382044; 104158; 104158
mouse subset 2 (f)
11818; 545936; 12914; 12915; 13030; 13649; 13866; 13867; 13982; 14176; 14784; 110006; 15126; 211323; 15439; 16362; 16363; 17967; 18127; 18205; 78405; 18212; 18393; 18538; 18673; 18709; 18791;
18815; 20020; 19288; 20198; 20200; 20194; 20437; 20375; 20779; 20927; 22117; 22370; 22596; 26903; 12848; 65962; 26941; 18786; 15111; 18777; 22193; 72432; 211652
mouse subset 3 (f)
232345; 11461; 71885; 11545; 11651; 16952; 11747; 11749; 11818; 11835; 109689; 11946; 11947; 12443; 545936; 12145; 12161; 12169; 12223; 12259; 12260; 12333;
12395; 12402; 12505; 12550; 12567; 12576; 12609; 12638; 100088; 67300; 12768; 12772; 12842; 12846; 232680; 12873; 76703; 56373; 70574; 12912; 12914; 12915;
12951; 26416; 12978; 14219; 12385; 12387; 13030; 13035; 13166; 13394; 13490; 72349; 13649; 66235; 50701; 56501; 13712; 13717; 13043; 13858; 13866; 13867; 13870;
13982; 26379; 23871; 14011; 14061; 14063; 14071; 14058; 14081; 74205; 50790; 14104; 14173; 14175; 14176; 14178; 14184; 14194; 14225; 14228; 14281; 56717; 26423;
14360; 14381; 14388; 622339; 14460; 14573; 14609; 12766; 14784; 14800; 56637; 110006; 15122; 15122; 15130; 15129; 15135; 15132; 544763; 15126; 15186; 15234;
211323; 217082; 627468; 15439; 16000; 16002; 16009; 16147; 16176; 16183; 16189; 16193; 16159; 16160; 16362; 16363; 27056; 16367; 16476; 16523; 16211; 16649;
16666; 16819; 16846; 17002; 17005; 17149; 17188; 17287; 17294; 17295; 17364; 17523; 17750; 17921; 17938; 17967; 18002; 18015; 18049; 68039; 18127; 18188; 18195;
18205; 78405; 18212; 209815; 18260; 76303; 18489; 18505; 18508; 18538; 18673; 16922; 20704; 20725; 30955; 18709; 18783; 18784; 18791; 18792; 18793; 18803;
18805; 18815; 18816; 18854; 20020; 18986; 19016; 19085; 18750; 18753; 26413; 26417; 23939; 22074; 19211; 19226; 19227; 19288; 19387; 19645; 19718; 69263;
106344; 627490; 110651; 20181; 20198; 20200; 20194; 20239; 20297; 20315; 66945; 67680; 66925; 20423; 20510; 20568; 20610; 170930; 20682; 20683; 20375; 20730;
20779; 20787; 20846; 20848; 20851; 56274; 58231; 20927; 21374; 21423; 21418; 21785; 21802; 21803; 21808; 21817; 21898; 21926; 22059; 22025; 22026; 22066; 22117;
22174; 546265; 22218; 22327; 269523; 22339; 22346; 22371; 22594; 22628; 12767; 19349; 382523; 53357; 26565; 13011; 56469; 56453; 22403; 18935; 16798; 12442;
12848; 65962; 16591; 26941; 18786; 15111; 19340; 66515; 228983; 60365; 50995; 20928; 74117; 66713; 16391; 100689; 18777; 22193; 17978; 13018; 107971; 19017;
108679; 72432; 24128; 18779; 72119; 211652; 230908; 69219; 12488; 16650; 71720; 50523; 54160; 26891; 85031; 433256; 70415; 64177; 70380; 194352; 28169; 68262;
104570; 80283; 81535; 74649; 64291; 79196; 71240; 216810; 76763; 11878
dog subset 1 (mp)
403643; 609623; 612604; 480110; 479159; 475491; 403520; 478347; 403669; 415120; 483319; 608477; 477699; 403551; 403550; 479787; 494010; 608452; 475474;
479912; 479888; 442987; 403453; 477046; 475666; 480372; 612159; 480425; 403869; 442971; 449023; 474442; 481278; 612603; 475631; 611458; 476437; 480113;
403968; 483369; 480878; 403708; 483318; 475175; 403945; 483307; 608659; 450237; 475913; 483317; 403694; 478702; 480621; 403643; 479011; 474830; 609393;
476022; 403677; 474722; 479004; 403612; 477198; 477601; 480953; 480360; 483311; 483313; 608918; 612710; 483310; 608610; 609568; 608519; 444855; 475473;
607420; 475334; 479354; 483316; 478701; 484056; 607088; 403435; 479704; 478547; 477858; 608340; 475381; 483312; 474637; 480897; 479949; 479587; 403684;
475851; 403580; 480818
dog subset 2 (mp)
478250; 481685; 476438; 403945; 403407; 477740; 478284; 484472; 478194; 475201; 475945; 442971; 490628; 484789; 487786; 479649; 611100; 486077; 608543;
403413; 403515; 442980; 403883; 489090; 403462; 478984; 476414; 475472; 475474; 403845; 478170; 474485; 487498; 403570; 478755; 483312; 491021; 491286;
474819; 479911; 403831; 480110; 479074; 478952; 474964; 480100; 403735; 480726; 476688; 403975; 403974; 403439; 403628; 478800; 486148; 403789; 489928;
403788; 483318; 477210; 489159; 442952; 484185; 482154; 403811; 403967; 477602; 479680; 403616; 450207; 481124; 478781; 488504; 476215; 403438; 490008;
609832; 482891; 484768; 444854; 448784; 607700; 403402; 488958; 477498; 477823; 490404; 403844; 492123; 448778; 607885; 480424; 477166; 487221; 403911;
442986; 442985; 606872; 484431; 478938; 487235; 609956; 606755; 486613; 482840; 485874; 488167; 479473; 482083; 403602; 611266; 403907; 480869; 475234;
609421; 480459; 609934; 611397; 485783; 481014; 475108; 610128; 475509; 477263; 486717; 477488; 483277; 477176; 490255; 609852; 486710; 403955; 484648;
609725; 479158; 477775; 403494; 476601; 612448; 480580; 403787; 480143; 475851; 607023; 403522; 480080; 609265; 488797; 481619; 490439; 492299; 476710;
484388; 485837; 483042; 490682; 403464; 403484; 485864; 612861; 490967; 484196; 484652; 486303; 482047; 403998; 481628; 477039; 485867; 487486; 403634;
487437; 403869; 478274; 490397; 481598; 484695; 490867; 485406; 489152; 485377; 478701; 492095; 479010; 478874; 488593; 485016; 608060; 403802; 484056;
476336; 480621; 491867; 607740; 493997; 609253; 611171; 477237; 480645; 489818; 475864; 488017; 489584; 479854; 481713; 403767; 484820; 481290; 483121;
475451; 492059; 487119; 478350; 476294; 479926; 488423; 488125; 480543; 478492; 490006; 609233; 485548; 611262; 489156; 490434; 477052; 484052; 477213;
475024; 474672; 475213; 609622; 608850; 490926; 478299; 480450; 481048; 491756; 488649; 476057; 479307; 489943; 479884; 483299; 482101; 609976; 476769;
607954; 486916; 610506; 610825; 479648; 474829; 487323; 487132; 485167; 478541; 491093; 608937; 476425; 480931; 479744; 483035; 487343; 488025; 481577;
483838; 485504; 479892; 480818; 609018; 479921; 483060; 476541; 484947; 485383; 477974; 474445; 487316; 490901; 478399; 481794; 474444; 485195; 478052;
474837; 477901; 484527; 607929; 483399; 475089; 476998; 611038; 484133; 606873; 477351; 479832; 479868; 481809; 484028; 474928; 476381; 476419; 612746;
487367; 478372; 612792; 483124; 486822; 484161; 612751; 607947; 611919; 477818; 486814; 481996; 476312; 490691; 476020; 612797; 481988; 477712; 484303;
609054; 484776; 477244; 478664; 610120; 481822; 477100; 479838; 474618; 478300; 479618; 477370; 612944; 490757; 482745; 610895; 478082; 488956; 608333;
480077; 475740; 484449; 479211; 607587; 480060; 490046; 610408; 483630; 488813; 476079; 612380; 486350; 475053; 607562; 480244; 403518; 478760; 489539;
476455; 611710; 489713; 485661; 481999; 487233; 485365; 483190; 608950; 481859; 475803; 478115; 609381
dog subset 3 (mp)
477115; 491292; 478895; 489828; 483322; 475923; 477587; 403580; 478250; 608134; 478637; 480369; 477236; 478489; 403960; 403410; 490385; 403962; 403910;
483701; 479666; 403551; 475251; 490878; 449021; 474808; 476600; 403550; 479787; 474787; 480622; 483021; 479476; 477753; 489486; 481685; 475877; 476322;
403435; 476094; 479325; 479246; 482620; 479139; 482558; 442936; 478982; 475680; 486699; 442969; 476438; 403945; 403407; 486469; 442968; 403588; 489993;
491857; 480487; 486562; 474823; 403954; 403670; 475924; 486666; 607262; 482897; 489461; 612911; 479450; 403669; 478393; 487981; 477740; 477101; 484490;
478284; 487718; 483763; 611781; 481744; 610190; 403523; 488143; 449028; 403416; 488770; 403618; 612349; 488124; 607482; 403762; 484472; 486607; 475867;
608552; 403437; 475526; 474180; 478194; 476728; 479348; 489221; 479556; 479555; 479354; 611933; 474584; 476694; 475201; 403908; 483745; 403567; 487899;
486896; 488473; 487432; 403474; 403980; 487746; 487044; 489369; 487965; 483845; 485338; 484610; 480059; 611695; 476485; 611674; 403646; 403469; 475945;
481595; 479329; 484483; 475992; 488997; 480527; 610456; 442858; 489775; 481131; 475537; 609920; 474890; 403429; 481563; 609010; 492302; 403483; 609767;
403827; 477796; 442971; 403971; 480578; 490628; 484790; 484789; 403632; 475380; 403651; 403824; 609516; 403739; 490269; 607922; 478806; 479866; 608125;
487786; 403856; 489188; 403923; 481276; 607605; 487649; 479331; 475928; 474510; 477185; 478108; 474844; 488922; 488008; 612240; 477032; 479649; 611100;
486077; 488577; 608543; 608843; 489664; 475121; 494010; 483038; 479740; 474177; 415122; 415120; 415128; 479740; 415127; 608452; 478365; 477807; 479353;
476623; 484142; 474873; 483976; 483106; 483626; 607439; 607589; 480472; 479448; 489573; 610935; 488036; 491319; 403413; 476715; 403701; 487984; 487849;
486276; 489117; 611653; 609526; 485839; 403657; 404306; 481528; 487421; 487870; 475590; 489468; 403515; 442980; 480466; 492129; 608908; 610283; 480113;
477373; 484943; 403883; 481105; 493995; 476105; 477747; 403640; 489287; 487761; 607474; 475511; 490153; 476993; 489090; 478711; 490261; 403619; 607277;
478927; 481018; 486102; 478970; 403462; 478984; 476414; 475473; 475472; 403915; 475582; 488808; 415125; 475474; 474544; 484713; 403727; 486025; 481464;
403845; 490792; 478232; 481387; 475032; 478170; 474485; 483808; 487548; 478572; 403755; 486079; 487498; 480495; 479885; 403721; 403418; 403570; 481056;
609822; 481126; 403943; 403912; 487351; 478755; 482275; 489469; 483312; 491021; 607755; 478770; 478047; 484024; 478575; 474819; 479912; 479911; 611366;
477556; 474585; 613006; 478327; 476881; 479714; 490883; 403831; 490630; 489372; 480110; 478034; 479074; 487309; 487551; 478952; 491198; 480348; 609531;
609564; 403170; 486068; 442987; 479864; 485884; 474964; 478450; 480100; 482370; 403735; 476411; 489758; 475124; 476276; 403612; 403612; 474850; 474680;
480726; 479406; 474697; 403979; 480438; 476688; 612371; 481689; 403909; 403975; 487737; 481558; 403801; 487739; 610255; 442951; 483664; 484067; 488516;
491618; 610316; 479967; 475166; 479122; 403782; 403974; 403785; 403985; 612271; 403545; 403850; 403628; 442990; 475574; 478381; 483665; 484990; 478800;
481500; 480015; 486148; 489208; 403789; 488429; 486493; 489928; 488437; 489929; 477956; 403788; 483318; 477210; 608977; 491368; 489159; 609423; 442952;
484185; 609429; 483002; 482154; 403811; 403967; 478666; 491042; 444857; 486522; 477602; 486513; 490976; 442954; 477029; 479680; 478151; 403830; 491320;
489126; 403616; 450207; 481365; 480124; 481124; 478781; 484787; 488643; 612899; 481360; 477700; 612436; 476070; 480144; 610902; 476196; 484932; 478275;
488504; 484781; 476803; 480488; 480218; 476160; 489368; 403537; 403693; 478939; 612759; 478061; 476262; 476215; 403438; 477683; 490008; 477019; 484504;
483732; 479417; 489428; 403733; 403445; 489432; 403885; 480346; 609986; 494006; 494002; 491924; 482891; 484768; 444854; 488828; 403768; 474729; 478230;
487873; 448784; 483653; 403745; 483995; 403924; 482978; 479836; 480646; 613013; 611315; 607700; 485677; 608445; 488693; 480021; 477535; 478813; 442859;
486858; 474897; 475450; 475312; 403402; 488958; 479051; 480558; 606932; 611645; 477498; 403822; 403784; 480676; 403780; 608196; 477823; 474472; 610981;
490404; 484147; 476822; 480117; 491866; 476749; 478343; 403844; 489870; 492123; 484666; 486694; 448778; 477501; 488746; 403476; 607885; 612814; 612316;
609680; 480424; 477166; 488573; 487221; 403911; 491597; 442986; 442860; 442985; 475823; 476371; 606872; 403879; 484431; 480422; 476177; 478938; 487235;
609956; 484963; 606755; 484018; 482840; 403426; 476446; 485874; 489692; 488167; 479473; 482083; 403602; 611266; 485903; 611484; 478840; 479751; 491023;
403907; 480869; 494001; 479483; 403659; 475234; 490360; 403606; 480533; 606861; 478337; 480344; 403608; 612199; 609421; 479351; 403556; 480459; 484774;
490904; 489968; 494005; 609934; 474176; 477575; 486758; 491690; 477746; 474652; 478464; 478347; 611939; 489547; 480454; 485004; 611397; 485182; 476104;
476402; 403408; 490382; 481014; 403832; 442942; 475108; 475509; 477263; 490563; 486717; 477488; 483277; 477176; 490255; 609852; 475389; 611326; 476439;
611060; 486710; 479082; 404008; 608699; 483974; 403955; 484922; 481656; 403568; 484648; 475875; 483881; 477045; 612298; 479158; 476915; 478149; 480854;
478127; 477775; 479446; 483729; 476664; 403494; 489798; 477308; 478667; 477499; 480181; 478328; 479949; 475080; 476601; 478356; 476804; 474722; 487353;
612448; 491768; 480580; 610499; 475381; 476206; 610820; 485010; 476823; 482963; 480141; 612326; 403787; 480143; 475851; 475852; 491615; 610295; 607023;
479564; 403981; 403522; 448790; 477945; 482977; 403999; 480080; 403994; 611673; 489508; 609265; 488797; 481619; 490439; 492299; 484830; 609304; 404000;
478171; 488006; 474673; 476710; 486407; 403896; 484388; 485837; 403559; 476258; 483042; 490682; 403464; 486507; 488405; 612159; 479781; 478471; 403484;
485864; 481228; 612861; 403993; 481272; 488449; 490967; 477240; 485940; 485088; 607840; 477965; 484196; 484652; 491950; 480815; 479370; 479173; 481802;
491683; 479197; 611017; 611193; 486303; 485079; 484064; 482555; 403412; 477072; 488989; 488213; 485941; 607720; 482047; 403998; 489932; 481519; 481628;
477039; 485867; 474355; 487486; 403601; 403599; 403816; 403752; 488605; 403922; 403634; 487437; 477229; 480525; 477044; 489537; 403869; 478274; 490397;
489620; 481598; 484695; 403521; 490080; 404019; 490867; 480688; 479883; 485406; 403973; 489152; 480167; 478918; 490228; 478701; 403695; 609156; 474536;
607417; 487509; 492095; 612742; 479010; 478874; 403574; 476060; 488593; 485016; 608060; 403982; 481590; 403802; 494000; 484056; 477991; 476336; 490848;
480621; 399544; 491867; 488409; 607740; 481625; 493997; 609253; 476521; 476447; 611171; 477237; 480645; 489818; 475864; 476031; 488017; 489584; 490323;
488559; 608147; 479854; 475295; 403767; 483724; 484820; 481290; 479747; 490099; 479955; 488309; 483169; 609673; 475424; 612125; 477892; 607683; 492059;
480955; 487119; 478350; 476294; 479926; 608508; 478210; 476512; 488125; 480543; 488390; 478492; 609233; 612452; 485548; 491084; 611262; 489156; 475120;
611694; 612481; 488471; 477052; 481708; 484052; 477302; 611468; 480253; 475024; 477576; 474672; 475213; 609622; 608850; 607042; 486680; 488742; 479865;
478299; 480450; 481048; 609634; 491756; 488649; 574011; 476057; 479307; 490263; 481657; 489943; 403683; 479884; 483299; 403625; 482101; 481241; 489497;
478472; 475000; 480923; 488651; 484822; 486916; 610825; 476708; 489642; 479648; 482715; 479515; 484354; 612510; 474829; 487323; 487132; 485167; 478541;
480064; 479783; 612867; 608844; 479985; 608937; 476425; 478648; 480931; 479744; 403702; 481396; 474674; 403791; 479322; 483035; 403689; 487343; 488025;
481577; 482039; 483838; 485504; 476150; 485523; 479892; 485674; 608864; 476730; 491231; 482049; 480818; 609018; 611055; 483060; 476541; 483780; 484947;
483728; 477974; 478018; 485848; 474445; 487316; 478399; 612760; 476087; 478063; 481794; 474444; 485195; 476418; 479127; 490548; 477239; 474837; 477901;
474452; 487713; 608268; 490685; 484527; 607929; 486338; 480597; 485192; 480322; 476998; 611038; 606873; 477186; 482813; 477351; 480695; 479832; 479868;
608555; 481809; 474670; 490874; 489012; 475787; 487751; 489390; 476381; 476419; 612746; 487367; 478372; 484349; 481266; 483124; 609776; 486822; 484161;
612751; 607947; 609080; 612153; 442950; 611919; 477818; 608341; 485962; 608833; 403519; 489925; 486814; 490127; 479812; 476312; 490691; 476020; 612797;
481988; 478739; 478455; 477038; 477712; 484303; 609054; 608855; 484776; 488088; 477244; 477820; 478664; 610120; 474618; 478300; 479618; 480054; 612944;
490757; 482745; 474644; 610239; 610883; 478082; 475385; 482744; 488956; 608333; 480077; 490273; 475740; 485819; 479211; 479132; 607587; 480060; 490046;
610408; 442958; 477653; 475445; 480447; 488407; 609002; 478128; 488813; 476079; 477190; 612380; 490564; 486350; 482475; 612601; 607562; 489289; 485046;
480244; 403518; 474422; 489539; 491170; 611710; 490726; 478830; 481999; 485365; 483190; 491356; 608950; 613012; 612085; 486723; 611023; 489523; 481859;
474830; 475803; 609381; 483893; 478702; 484031
rat subset 1 (mp)
rat subset 2 (mp)
29275; 25377; 25728; 287774; 54226; 408242; 297566; 24223; 81613; 298566; 24235; 360380; 24854; 25627; 117029; 25419; 24291; 117184; 64529; 290257; 25633;
24331; 299606; 25437; 24337; 304929; 306761; 295279; 498277; 289211; 304966; 29558; 24366; 24367; 287375; 25661; 25019; 171128; 394266; 25062; 81504; 84427;
296654; 57298; 64352; 24434; 117056; 29424; 155012; 117280; 502373; 171016; 25617; 361384; 25464; 24494; 25325; 54259; 114517; 25467; 25021; 25724; 305506;
363577; 84598; 24514; 64030; 300242; 24530; 25608; 24536; 299858; 25595; 365057; 85483; 498638; 24566; 300999; 114553; 310738; 25494; 307982; 59109; 83497;
317576; 29665; 29431; 65051; 25737; 24627; 497724; 361310; 64303; 81531; 25344; 289021; 29741; 24650; 287925; 25692; 25738; 25096; 25097; 64204; 85253; 287527;
368070; 84024; 56782; 29340; 24686; 25614; 29222; 24924; 24697; 116689; 25622; 360370; 24699; 114767; 312703; 363875; 366957; 24703; 362161; 25676; 25703;
24716; 364985; 117269; 83840; 24615; 85247; 81778; 60352; 81780; 29259; 303468; 305450; 362513; 85385; 171379; 361574; 362242; 313845; 85384; 83805; 501099;
25125; 25155; 24949; 29179; 24817; 89804; 24826; 59086; 29591; 81810; 56083; 25625; 497808; 24842; 296099; 500450; 362788; 292925; 287208; 498736; 116484;
298317; 361585; 301442; 25156; 296603; 83785; 54319; 25696; 29169; 317371; 317676; 290805; 24883; 24919; 56011; 29753; 56010; 25576; 25578; 58820; 362558;
360471; 294250; 80848; 309072; 306347; 29130; 312492; 307766; 315350; 364786; 300772; 310806; 311130; 292645; 246334; 286975; 362045; 252971; 58945; 29376;
246756; 303068; 57025; 301164; 84018; 362249; 294568; 360526; 361420; 360640; 296121; 64520; 83533; 363466; 83631; 361724; 64193; 116501; 59114; 300057;
56725; 300367; 79252; 293524; 25414; 309595; 116673; 291309; 192363; 266601; 84477; 304017; 287622; 362293; 308384; 54227; 362686; 303882; 362501; 294981;
306141; 287155; 83427; 25514; 295378; 65037; 291150; 363026; 308939; 307189; 299809; 117268; 303634; 288305; 316213; 314850; 117185; 307474; 192277; 312916;
292763; 363140; 293619; 171159; 29372; 171039; 361196; 294864; 290704; 499250; 363544; 316228; 308267; 302983; 293476; 362938; 497836; 246146; 171071;
289508; 83718; 363067; 295338; 309374; 290556; 259275; 171350; 292846; 81511; 500449; 64031; 499356; 314941; 114590; 171562; 84357; 287659; 299976; 297597;
301013; 85311; 301009; 296360; 360734; 292041; 266688; 114858; 94272; 300944; 304714; 305544; 499884; 498232; 292064; 500282; 64531; 114246; 365661; 364755;
298646; 360866; 291369; 302963; 311192; 305391; 311703; 360968; 83582; 363292; 85265; 308516; 311055; 303190; 307585; 292735; 297822; 307850; 367160; 286965;
56763; 60564; 361994; 170843; 365895; 361612
rat subset 3 (mp)
116632; 116631; 57300; 311860; 25287; 50681; 29437; 25365; 81822; 29275; 303742; 361925; 81634; 24165; 24172; 25369; 29316; 25591; 24173; 24176; 25238; 171494;
24177; 81638; 25690; 24185; 25233; 25374; 24186; 24189; 24190; 24191; 266802; 287454; 25290; 25377; 60668; 25380; 56611; 25673; 79125; 78963; 24205; 29339;
79116; 25649; 54225; 25383; 292697; 25728; 287774; 54226; 408242; 246097; 25385; 24208; 81642; 64363; 64310; 140940; 29221; 117273; 64373; 360678; 362456;
25388; 84431; 24210; 29403; 25389; 300711; 65262; 171374; 94271; 360716; 297566; 83625; 24223; 304127; 64639; 297994; 116502; 64557; 24887; 65036; 58919;
24224; 170929; 24888; 303836; 25295; 81613; 64625; 308755; 116599; 25644; 140923; 29884; 497672; 114486; 298566; 24235; 362119; 24237; 117517; 298288; 313421;
497751; 313495; 50663; 64202; 360380; 29144; 29153; 25166; 25402; 83584; 64026; 64044; 58918; 24248; 25404; 50662; 362489; 500985; 171136; 114494; 25729;
288620; 308501; 500318; 25660; 171369; 25406; 313498; 25599; 64315; 54237; 360621; 307511; 83502; 307618; 94201; 140908; 114483; 114851; 83571; 25164; 24252;
24253; 24255; 116700; 140583; 24260; 309361; 24854; 65160; 54241; 25627; 60463; 117029; 58936; 29393; 84352; 499431; 294027; 289395; 81646; 497663; 54245;
287942; 25419; 81649; 307403; 116630; 497822; 298518; 315707; 113927; 64462; 58822; 116549; 307641; 114122; 29317; 288067; 29382; 64032; 84353; 24291; 117184;
64529; 25424; 290257; 29175; 116639; 79129; 300047; 24296; 24297; 171352; 361523; 246070; 293985; 29277; 29296; 171522; 25086; 171352; 286929; 24307; 29680;
25146; 79128; 114489; 306722; 140926; 25311; 29167; 311186; 25112; 29467; 29318; 287765; 300710; 298298; 24314; 317189; 25252; 25312; 140694; 25633; 84350;
24318; 29238; 314616; 307563; 25254; 114856; 311406; 497778; 399489; 287114; 29565; 25313; 24329; 24330; 366492; 117045; 116636; 287986; 287444; 24331;
299606; 363854; 302811; 366142; 25043; 25437; 60465; 170915; 25315; 65030; 24336; 24337; 29496; 291703; 306274; 24356; 304063; 303828; 312299; 29251; 25584;
304929; 29243; 306761; 60327; 289055; 24360; 24361; 114024; 113976; 50646; 25047; 295279; 498277; 289211; 304966; 29558; 361969; 24366; 54250; 29348; 84489;
25022; 24367; 63839; 294515; 287375; 306204; 54251; 497666; 25661; 314322; 56718; 25449; 25320; 25150; 25019; 361388; 502664; 29423; 24383; 54254; 171128;
24387; 27100; 25235; 24392; 394266; 25236; 25283; 140589; 81666; 60429; 24400; 313017; 25062; 117549; 303248; 81504; 84427; 498416; 497769; 24413; 24414;
84027; 296654; 24421; 501110; 24423; 57298; 64352; 499689; 24424; 29438; 25260; 29487; 366547; 83830; 83828; 361580; 353256; 299347; 24434; 171528; 500987;
117056; 79224; 29424; 297893; 24443; 155012; 303310; 29560; 289623; 499092; 29395; 24451; 79239; 25735; 79256; 117280; 502373; 171016; 60421; 25117; 64441;
65028; 294254; 294254; 266759; 25617; 291671; 24471; 299331; 301252; 361384; 313641; 25075; 24476; 25464; 304988; 298205; 24481; 25712; 360697; 24482; 25718;
24483; 25151; 25662; 24484; 25285; 83476; 360922; 297767; 24493; 24494; 287287; 24498; 24499; 25205; 116558; 25325; 116553; 66029; 306626; 24506; 54259; 24954;
114517; 24508; 364081; 25467; 170921; 360606; 311144; 315346; 25021; 296456; 499271; 24511; 309684; 25724; 305506; 50693; 363577; 60583; 84598; 24514; 24516;
29553; 64030; 288001; 24917; 24525; 300250; 300242; 360625; 360626; 140433; 24530; 313050; 94106; 170496; 155918; 25608; 24536; 25477; 60374; 299858; 301034;
311337; 289227; 501502; 171577; 312689; 294446; 297176; 29357; 25631; 50554; 361378; 25152; 25595; 367171; 29457; 291439; 78967; 60430; 29333; 314856; 304798;
311311; 116667; 308106; 365057; 171341; 85483; 291234; 497834; 308463; 309168; 315606; 300339; 81686; 171045; 25335; 81687; 266713; 303413; 64046; 81709;
81521; 498638; 24566; 300999; 81710; 362657; 310900; 286918; 498982; 24577; 298894; 65261; 288770; 85482; 114553; 300955; 25135; 308690; 25744; 83619; 81736;
309452; 29508; 25336; 25337; 310738; 25494; 499194; 191575; 291084; 314969; 24599; 24600; 25496; 296136; 300303; 307982; 29541; 59109; 25054; 81738; 289323;
302552; 25502; 83497; 25503; 317576; 81528; 140914; 29665; 29542; 497733; 24617; 29431; 29432; 56816; 29700; 65051; 25737; 289019; 24627; 25266; 25267; 497724;
65144; 308321; 361310; 64303; 81531; 24891; 25344; 24648; 116589; 289021; 29741; 298696; 24650; 25157; 25692; 25619; 25738; 29337; 25096; 25097; 64204; 85253;
287527; 296371; 315713; 494322; 288479; 368070; 29240; 363633; 24664; 84024; 171068; 25664; 497991; 64367; 24666; 24672; 65179; 56782; 65248; 25636; 29699;
24680; 25023; 170538; 29340; 360748; 116590; 50689; 54268; 50658; 25272; 170851; 58960; 303200; 114495; 363855; 308845; 25268; 29245; 314615; 29192; 81751;
89830; 50557; 29527; 25614; 24924; 24697; 117063; 116689; 25622; 360370; 24699; 114767; 360406; 29616; 29498; 312703; 64192; 64633; 50645; 84590; 363875;
366957; 361381; 298012; 499870; 24703; 81757; 116546; 24706; 310486; 25676; 24708; 313048; 83712; 81758; 25703; 315655; 309165; 24716; 116468; 288414; 288003;
304528; 81762; 300807; 81763; 287417; 364985; 140661; 140654; 29304; 81771; 117269; 501560; 83840; 65136; 65139; 161477; 50718; 365320; 362720; 295214; 24615;
85247; 81778; 445415; 25742; 60352; 25541; 287562; 81780; 29538; 25615; 83841; 25544; 29259; 25651; 303468; 305450; 362513; 85385; 114613; 24898; 29464; 24782;
24784; 60577; 84386; 171379; 361825; 64301; 361574; 362242; 24786; 24787; 313845; 85384; 24790; 367846; 24791; 24796; 25353; 83805; 300095; 501099; 24797;
497823; 25124; 25125; 311622; 261730; 314621; 116470; 361256; 25155; 24949; 29179; 317256; 373541; 24811; 294810; 503174; 316221; 363309; 361255; 302711;
117526; 24817; 171046; 24818; 89804; 64523; 301965; 83474; 306862; 362280; 361178; 24826; 24827; 59086; 84574; 116487; 29591; 81810; 56083; 83580; 81812;
24831; 116510; 292994; 289337; 24835; 25625; 497808; 29388; 64550; 361100; 303194; 24842; 296099; 362675; 500450; 54314; 29268; 362862; 362788; 60445; 24855;
292925; 25653; 287208; 24856; 316531; 500363; 498736; 252921; 85489; 116484; 58819; 29261; 25232; 298317; 81920; 361585; 301442; 24860; 25575; 25156; 296603;
25361; 116643; 83785; 24874; 54319; 81818; 25696; 362779; 24877; 29169; 116669; 317371; 117538; 317676; 313235; 290805; 24883; 312560; 84495; 24919; 56011;
29753; 56010; 25576; 25578; 301348; 58820; 362558; 170672; 29619; 64158; 360471; 296911; 80848; 25445; 306347; 29130; 310678; 288471; 79257; 360908; 64647;
304323; 314764; 117274; 497801; 361181; 315350; 26295; 364786; 300772; 310806; 171445; 292645; 65137; 246334; 286975; 362045; 58945; 29376; 140668; 246756;
303068; 300046; 246775; 266610; 500715; 117279; 301164; 294235; 84018; 295052; 302671; 290215; 294568; 287930; 360526; 361420; 296566; 312790; 308761;
360481; 296121; 64520; 83533; 116642; 363466; 83631; 361724; 64193; 114505; 29282; 116501; 83504; 59114; 246253; 300057; 366962; 114523; 312382; 297954;
84469; 66015; 79252; 29455; 293524; 140665; 298655; 25414; 116598; 54299; 408210; 308016; 309595; 116673; 291309; 192363; 266601; 84477; 304017; 304914;
64681; 501506; 64547; 300219; 295543; 362293; 308384; 116721; 54227; 81732; 289820; 64012; 294010; 171092; 362686; 116698; 303882; 362501; 78969; 294981;
306141; 311030; 287155; 315958; 500929; 296554; 299923; 83427; 25514; 308416; 65037; 291150; 497739; 363026; 192218; 307189; 307081; 362248; 299809; 117268;
288305; 310344; 83722; 316213; 314850; 117185; 83516; 84014; 311621; 286988; 192277; 312916; 299783; 304135; 362960; 503027; 292763; 363140; 114212; 124461;
84479; 140860; 302248; 29372; 171039; 294864; 311546; 290704; 192348; 499250; 363544; 293455; 298066; 316228; 302983; 117243; 314465; 309757; 362938; 297381;
304073; 29392; 246146; 171071; 289508; 83718; 363067; 292848; 300066; 295338; 299061; 309374; 259275; 171350; 292846; 81511; 500870; 313228; 500449; 64031;
361767; 171144; 140915; 58978; 282817; 499356; 362021; 25435; 114590; 171562; 84357; 287659; 299976; 289388; 291137; 360915; 252902; 297597; 301013; 85311;
300126; 301009; 286937; 296360; 94340; 360734; 292041; 114858; 305544; 499884; 498232; 292064; 304887; 500282; 64531; 114246; 287273; 245956; 291135; 365661;
500533; 116469; 364755; 298646; 360866; 171358; 246273; 298784; 305392; 291369; 302963; 287164; 64803; 299706; 362789; 311731; 367083; 305391; 296278;
311703; 360968; 362596; 362393; 363292; 315546; 314638; 85265; 308516; 299828; 303190; 307585; 303277; 297822; 366668; 498234; 295692; 286965; 60564; 361994;
192126; 171158; 171018; 499702; 501702; 170843; 29214; 365895; 24422; 361612; 363974; 291081
mouse subset 1 (mp)
104158; 50529; 627889; 15078; 66475; 66234; 52538; 28250; 26395; 11947; 56448; 70450; 22378; 232345; 11576; 11657; 11674; 13077; 13120; 382044; 99571; 14862;
14863; 14651; 15368; 17250; 66480; 18263; 20493; 20692; 20714; 20716; 22059; 12759; 20964; 404195; 15483; 17105; 19293; 17117; 14081; 11287; 11812; 13849;
22095; 12944; 18453; 18416; 13039; 192897; 280662; 11818; 432613; 15374; 445007; 170472; 14635; 73710; 22370; 104158; 22608; 22154; 14528; 12631; 20091;
623245; 20219; 432798; 193740; 545302; 16668; 13629; 14618; 17837; 73834; 67283; 71947; 21815; 68337; 260302; 69674; 217325; 57230; 217331; 14161; 53872;
230751; 12279; 217328; 22151; 14865; 22350; 16427; 12306; 14864; 109900; 67891; 68193; 18242; 27276; 20116; 15160; 14784; 17319; 12583; 18555; 627490; 19934;
27207; 20194; 11461; 68312; 14694; 627160
mouse subset 2 (mp)
11464; 11699; 11816; 11818; 11820; 16617; 11973; 12010; 545936; 12259; 12321; 333883; 12759; 17228; 12774; 12944; 12954; 66473; 109660; 13030; 13035; 13419;
109901; 50701; 13726; 13866; 14067; 58992; 14129; 14131; 14130; 246256; 14132; 110135; 99571; 14248; 14268; 14375; 320415; 14619; 14571; 14784; 14786; 107746;
227753; 14864; 14866; 110006; 15078; 15194; 12628; 15381; 51810; 15461; 94175; 14828; 81489; 15894; 16176; 16185; 16153; 16331; 16403; 16367; 16399; 16409;
16416; 192897; 16418; 16428; 16451; 16452; 16542; 16590; 16623; 110310; 16816; 16846; 16847; 16971; 14725; 17756; 26408; 17295; 12265; 20288; 15235; 19882;
17829; 17969; 18049; 18073; 18125; 18175; 18211; 18260; 320634; 18406; 18439; 18479; 268591; 18538; 238871; 18588; 18596; 67199; 18643; 18645; 18673; 240752;
18708; 18709; 18763; 67451; 18791; 18803; 18805; 18806; 18810; 18815; 18816; 18949; 18968; 18979; 19073; 19084; 18754; 19106; 19122; 53380; 14083; 19231; 19242;
19246; 15170; 19247; 19248; 19262; 19264; 19267; 19305; 19353; 19354; 110157; 19400; 218397; 19662; 19713; 545683; 20112; 20198; 20200; 20194; 29857; 20304;
20343; 20391; 24055; 230126; 20416; 20537; 20586; 20637; 20648; 20662; 20663; 20682; 381358; 20779; 20807; 20848; 20963; 20964; 20965; 21405; 21687; 21819;
21803; 21812; 21813; 21817; 21825; 21937; 21938; 22059; 27223; 209456; 22004; 621054; 22031; 22088; 66827; 22141; 22151; 22166; 22209; 546265; 22215; 22218;
22324; 22325; 22339; 22350; 22359; 22370; 22376; 56743; 22427; 22431; 22632; 54401; 22627; 22628; 22629; 22631; 22661; 16975; 252870; 53761; 65969; 11489;
13716; 27204; 26903; 93762; 16370; 241226; 56469; 229776; 23992; 232889; 22061; 78473; 12703; 56455; 384783; 71609; 11492; 11490; 21848; 18519; 20975; 266692;
83767; 18452; 73178; 21341; 21340; 53859; 84092; 116870; 26926; 194590; 21945; 23942; 30939; 65962; 26941; 79456; 56724; 234959; 11504; 67384; 29810; 213539;
12927; 240087; 20970; 98910; 67952; 14389; 28185; 16923; 56459; 59026; 11867; 12607; 242687; 51789; 106021; 24063; 24064; 56424; 14694; 18777; 57257; 17289;
101206; 59035; 233726; 12461; 20218; 14674; 12469; 107971; 327826; 50877; 56070; 20867; 26754; 26411; 56289; 56460; 22770; 19414; 19894; 56541; 12465; 233789;
76779; 78309; 68652; 30957; 74204; 105782; 12488; 66245; 17872; 68098; 29876; 20466; 20399; 19260; 56642; 30877; 20409; 18458; 83382; 16797; 54390; 18569;
17060; 239393; 56085; 50527; 58859; 58194; 192157; 382985; 28019; 243816; 66940; 85031; 80987; 78928; 67838; 80707; 235442; 20418; 224796; 24127; 106298;
216549; 58800; 240754; 53325; 74195; 67166; 73247; 24071; 64177; 94212; 59079; 67475; 11610; 240880; 116871; 232944; 69581; 13418; 621205; 240047; 230649;
381410; 66999; 73251; 252966; 103724; 52589; 20017; 66262; 16475; 52428; 66860; 246782; 63955; 192192; 60599; 72544; 319161; 74020; 223513; 210710; 218543;
60510; 171388; 73523; 170771; 15257; 234577; 320452
mouse subset 3 (mp)
17961; 17960; 67758; 11350; 11363; 11423; 11430; 11459; 11475; 11461; 11464; 71885; 56456; 109711; 11486; 11522; 11540; 11541; 11545; 11548; 11555; 110355;
11765; 11576; 11608; 11622; 11651; 11652; 17025; 11655; 11657; 11674; 230163; 11676; 11682; 11684; 11689; 11686; 11699; 218038; 16952; 12306; 11747; 11749;
11750; 11783; 11789; 20219; 11792; 11799; 11807; 238055; 11810; 11813; 11816; 11818; 11820; 16617; 14102; 14103; 11835; 27421; 11836; 11840; 11842; 11846;
11848; 11852; 192662; 11857; 216869; 11886; 11889; 11890; 11910; 11920; 11946; 11947; 11957; 11964; 11973; 26362; 12010; 12013; 12015; 12017; 12018; 12021;
12028; 12038; 12443; 12043; 12047; 12048; 12051; 12053; 110279; 12096; 545936; 12122; 12144; 109778; 12161; 12177; 51800; 12189; 12190; 53414; 12259; 12266;
15139; 12274; 230558; 110382; 12279; 230099; 12314; 12317; 12321; 12330; 12333; 12362; 12367; 12368; 12369; 12370; 12371; 12359; 12389; 12395; 12402; 208650;
12428; 12447; 12466; 12527; 12483; 21940; 12487; 21939; 12505; 333883; 12517; 16149; 12518; 12476; 12534; 23834; 12532; 12550; 12562; 12567; 12568; 12571;
12575; 12576; 12606; 12608; 620772; 12638; 12640; 12649; 12671; 12675; 12759; 12729; 67300; 17228; 12772; 12774; 12780; 12795; 12842; 12843; 12817; 12821;
12902; 12912; 11909; 12914; 12928; 12929; 12944; 12954; 26416; 12978; 12981; 12984; 12986; 12988; 93687; 104318; 27373; 12995; 13000; 13001; 13003; 13008;
13016; 14219; 12387; 66473; 109660; 13030; 13034; 13035; 13038; 13047; 13057; 66445; 13076; 13077; 13078; 13113; 13088; 13095; 72303; 72082; 13098; 56448;
13106; 13113; 337924; 13120; 13070; 13074; 13132; 13138; 69635; 13163; 13176; 13191; 107986; 13197; 13198; 13202; 13207; 77591; 74754; 18104; 54004; 13383;
13406; 13429; 13419; 13433; 13489; 13490; 13496; 13507; 109620; 13517; 14357; 19252; 13537; 110074; 13555; 13560; 13629; 13645; 13649; 13653; 13836; 13684;
13685; 208643; 276770; 109901; 50701; 15568; 56501; 13711; 13717; 13726; 13043; 328572; 13849; 13850; 13857; 13866; 13867; 13871; 13872; 319955; 13982; 23871;
23872; 104156; 14056; 14061; 14066; 14067; 14058; 58992; 74145; 14060; 14080; 14088; 74205; 50790; 19229; 14125; 14129; 14131; 14130; 246256; 14132; 14161;
110135; 14173; 14178; 14182; 14184; 14183; 99571; 14198; 14200; 56484; 14248; 286940; 14254; 14255; 14257; 14268; 14281; 56717; 14309; 14459; 14360; 14375;
14388; 69976; 14432; 622339; 14463; 320415; 14580; 11692; 14600; 14609; 14619; 14734; 14629; 14632; 14682; 14683; 14688; 55948; 14571; 14763; 216860; 14784;
14786; 14783; 50915; 14815; 14816; 56637; 227753; 14862; 14864; 14866; 14865; 14863; 14871; 14872; 17688; 83602; 14884; 14886; 72308; 110006; 14939; 15270;
15078; 15115; 15160; 15194; 433759; 15186; 12628; 15248; 15251; 53323; 217082; 433238; 627468; 15368; 15369; 15378; 225307; 15381; 51810; 15402; 15461; 94175;
15469; 15484; 15499; 15500; 15502; 193740; 193740; 15482; 15525; 14828; 624853; 15526; 15507; 15519; 15516; 81489; 15551; 21923; 15874; 15894; 15951; 15974;
15976; 15977; 15978; 15980; 16000; 16001; 16002; 16004; 16008; 16009; 16011; 16007; 16069; 19664; 16175; 16176; 16189; 16193; 16194; 16195; 16198; 16153; 16163;
15930; 26356; 16334; 16331; 16337; 16403; 16362; 54139; 16367; 16398; 16399; 16400; 16401; 16402; 16409; 16410; 16411; 16412; 16414; 16416; 192897; 16418;
16419; 16426; 16428; 16447; 16451; 16452; 16476; 16527; 16542; 16590; 16623; 16644; 16211; 16653; 16678; 110308; 110310; 16691; 268482; 16669; 16776; 432548;
16816; 16818; 16819; 16822; 16846; 16847; 16867; 16905; 16971; 14725; 17002; 17005; 17085; 20400; 17075; 17113; 17118; 56150; 17126; 17127; 17128; 17159; 17756;
17758; 17760; 17190; 17196; 84004; 17210; 17221; 17246; 17248; 17536; 26401; 26407; 26408; 17295; 56615; 12265; 17345; 17350; 269881; 26403; 214162; 83995;
17390; 17392; 17393; 17395; 17420; 17523; 17535; 17685; 17698; 20288; 15235; 19882; 17701; 17750; 66902; 17769; 17777; 17829; 623609; 17858; 17863; 17869;
18109; 17880; 17913; 17938; 27354; 17969; 17973; 17975; 17984; 18016; 18024; 18033; 18034; 18044; 18045; 18046; 18049; 18073; 68039; 18102; 18105; 110109;
18125; 18126; 18127; 18128; 53885; 18175; 18207; 18211; 18212; 67459; 209815; 18245; 18247; 18260; 18261; 320634; 18294; 108078; 18406; 18439; 624438; 18550;
18787; 18479; 224105; 50873; 13180; 268591; 18538; 18557; 238871; 18588; 18590; 18595; 18596; 59020; 18616; 67199; 18643; 18645; 18671; 18670; 18673; 20704;
20724; 240752; 18708; 18709; 23988; 18763; 18791; 18792; 18793; 18803; 234779; 18805; 18806; 18810; 18815; 18816; 18830; 18854; 227099; 18861; 18948; 18949;
18968; 18970; 20020; 18976; 18979; 18986; 19016; 19014; 622262; 19035; 19042; 19052; 19060; 19073; 105787; 18747; 19084; 19087; 18750; 18751; 18753; 18754;
19090; 19091; 26413; 26417; 225724; 26419; 26420; 26414; 26395; 26396; 26397; 26399; 26400; 19106; 72981; 19123; 19144; 19152; 19164; 19165; 53380; 19206;
19211; 19225; 14083; 19242; 19246; 19255; 15170; 19247; 19248; 19262; 19264; 19267; 19268; 19274; 19286; 19305; 110391; 271457; 11891; 80718; 19353; 19354;
19358; 19359; 19361; 110157; 56044; 64143; 218772; 109222; 218397; 19645; 19646; 245688; 19651; 19662; 19684; 19697; 19704; 19713; 19718; 69263; 106344; 19877;
19883; 627490; 624324; 545683; 56040; 27050; 623245; 20111; 20112; 110651; 58988; 20116; 20042; 625298; 54127; 20133; 20135; 20193; 20198; 20200; 20194; 20203;
29857; 394252; 20280; 20293; 20304; 20297; 15529; 53378; 20339; 20343; 20344; 20345; 26398; 20391; 24055; 230126; 20416; 20537; 114479; 13162; 21366; 20544;
105243; 57738; 30805; 20568; 20586; 20587; 20595; 20637; 20648; 545845; 20656; 20662; 20663; 20682; 20683; 20687; 20692; 20737; 20750; 381358; 20779; 20788;
20807; 20604; 20607; 20846; 20848; 58231; 20878; 20869; 20907; 225115; 20963; 20964; 20965; 270627; 545600; 21354; 110960; 620313; 72726; 108903; 70430; 21372;
21374; 21405; 21423; 21454; 21687; 21745; 21752; 22041; 21780; 21418; 21420; 21781; 21819; 21802; 21803; 21804; 21810; 21812; 21813; 21817; 21824; 21825; 21833;
21834; 21857; 21872; 53791; 21926; 21937; 21938; 21952; 21969; 21973; 21974; 21975; 22059; 27223; 209456; 22062; 22004; 621054; 22018; 17229; 22027; 22031;
56191; 64930; 22084; 22088; 22094; 66827; 22139; 22145; 22147; 22151; 103733; 22160; 22166; 50493; 22171; 22174; 22209; 56550; 66105; 546265; 22215; 22218;
22227; 22287; 22324; 22325; 22329; 269523; 22339; 22346; 22350; 22352; 22359; 22367; 26950; 22370; 22371; 22376; 215280; 56743; 107684; 22427; 22431; 22591;
22594; 22632; 54401; 22627; 22628; 22629; 22631; 22637; 22668; 22661; 16975; 170484; 12227; 22142; 252870; 64213; 65969; 14283; 13716; 27204; 382523; 100683;
12005; 12545; 319182; 17120; 216238; 23957; 18201; 99375; 16370; 108155; 241226; 56469; 229776; 21393; 110198; 232889; 56505; 22061; 78473; 27413; 56455;
212647; 384783; 76408; 71609; 11492; 14731; 22035; 14082; 21848; 12454; 12633; 18519; 15937; 20975; 12427; 108012; 11766; 83767; 21976; 18452; 73178; 21341;
21340; 69454; 232431; 233406; 83945; 84092; 116870; 26926; 434203; 194590; 21945; 30963; 23942; 30939; 17164; 16600; 65019; 65962; 16591; 26941; 11450; 79456;
17444; 18208; 26357; 57756; 94275; 240913; 11504; 23886; 29810; 19340; 100198; 12927; 12304; 20185; 56640; 229933; 240087; 20970; 98910; 67952; 14389; 28185;
29809; 78388; 56805; 12125; 16923; 78733; 329777; 56459; 27416; 59026; 11867; 74117; 66713; 19360; 54130; 27041; 12607; 21849; 242687; 51789; 106021; 211770;
24063; 24064; 56324; 239273; 56424; 20842; 22143; 22153; 227613; 17988; 14694; 100689; 18777; 53333; 17289; 101206; 53319; 59035; 81601; 69617; 19124; 12461;
20218; 12469; 20873; 20620; 107971; 327826; 50877; 20174; 19017; 19765; 67145; 108767; 20867; 26754; 73690; 23794; 13404; 399510; 26411; 56289; 50883; 23970;
217030; 101488; 18074; 19414; 19894; 12465; 72119; 55982; 14269; 233789; 76779; 74105; 76299; 78309; 30957; 216739; 21981; 93759; 105782; 22377; 56173; 23821;
66245; 17872; 68098; 29876; 20466; 626834; 106039; 19260; 18094; 56642; 20409; 18458; 83382; 16797; 105837; 170770; 16348; 54390; 18569; 56626; 108115; 108116;
28253; 66824; 17060; 76123; 13631; 56085; 50527; 58859; 58194; 192157; 382985; 14373; 57441; 26891; 235661; 28019; 243816; 66940; 85031; 29870; 80987; 58869;
78928; 433256; 67838; 80707; 20418; 216549; 58800; 240754; 53325; 78255; 67166; 24071; 64177; 52530; 235281; 66834; 59079; 66233; 194352; 67475; 11610; 240880;
215303; 116871; 228775; 69581; 268973; 13418; 621205; 240047; 69191; 230649; 70839; 232560; 69736; 68520; 74616; 102580; 109151; 66999; 73251; 68559; 252966;
103724; 74178; 69538; 66262; 208677; 16475; 52428; 68876; 246782; 63955; 276846; 60599; 319161; 217674; 68724; 69482; 223513; 60510; 171388; 69601; 73523;
67379; 170458; 269800; 118449; 73139; 170771; 22154; 15257; 14857; 320452; 210356; 73710; 69912
dog subset 1 (i)
474177; 485069; 476927; 486846; 483763; 481255; 610255; 483664; 480812; 610316; 475920; 474890; 479303; 485069; 481528; 610120; 404306; 483245; 484927;
484757
dog subset 2 (i)
403416; 488770; 403618; 475537; 403429; 481563; 479866; 477185; 474844; 480472; 484943; 477686; 475356; 403883; 481105; 611946; 403640; 486102; 403754;
477295; 490792; 474485; 483808; 442957; 483312; 491021; 607755; 478770; 479074; 487309; 482861; 480348; 480438; 481689; 442951; 490069; 480812; 488516;
491618; 608158; 610316; 607374; 608559; 481051; 403439; 609293; 607422; 485209; 484990; 486493; 609429; 477602; 486513; 490977; 477601; 610902; 476196;
403537; 403693; 415126; 491946; 448784; 403924; 485069; 488447; 481122; 485677; 485930; 403692; 475083; 607885; 477166; 442985; 478938; 481772; 487235;
609956; 480648; 481513; 485874; 479473; 482083; 611500; 482686; 481756; 403606; 480533; 403609; 491303; 403556; 480459; 490904; 477746; 480338; 475108;
477263; 486717; 477488; 483617; 479158; 476915; 483729; 475851; 608403; 490439; 476710; 478478; 478336; 474657; 483042; 490682; 486507; 478471; 485864;
488449; 490967; 490968; 490969; 477072; 490163; 487486; 403752; 483343; 403869; 489620; 485406; 485016; 608060; 480621; 477237; 480645; 475864; 607083;
483724; 479955; 481319; 490395; 478492; 490006; 478595; 488195; 479428; 477302; 490926; 442949; 486133; 480147; 477664; 485017; 488025; 479921; 611951;
484198; 607060; 442959; 484527; 612343; 480709; 609312; 477864; 490648; 479734; 476693; 611936
dog subset 3 (i)
477699; 491292; 475923; 478757; 486537; 476140; 485590; 477603; 478321; 490385; 403910; 483701; 484375; 475994; 480605; 490878; 476094; 482620; 482558;
442936; 486469; 442968; 403588; 475176; 607073; 403954; 403670; 486545; 612911; 479450; 483763; 481744; 403523; 449028; 403416; 488770; 403618; 488124;
403905; 486607; 481947; 477162; 477270; 489077; 491750; 607408; 608552; 403437; 475526; 482682; 480416; 474584; 476428; 612941; 489050; 474684; 403567;
488007; 403474; 403980; 484671; 487366; 489369; 487965; 485333; 485338; 475007; 608847; 484610; 478442; 607076; 486241; 403469; 403468; 478552; 488997;
484780; 442858; 480169; 481131; 475537; 609920; 491325; 474890; 403429; 481563; 475359; 611216; 609346; 486028; 442971; 448802; 403651; 403826; 478835;
479866; 403856; 611795; 487649; 477185; 474844; 479991; 477032; 486077; 415128; 479740; 608452; 403904; 610668; 480472; 607766; 483100; 607007; 485839;
403657; 404306; 481528; 479219; 486121; 483112; 475590; 481565; 480466; 491860; 442955; 404002; 484943; 477686; 475356; 403883; 481105; 488508; 612282;
611946; 403640; 403639; 488942; 488940; 490153; 607661; 486102; 476063; 403754; 403915; 475582; 488808; 415125; 474544; 477295; 403845; 490792; 484445;
608412; 478232; 479746; 474485; 483808; 403433; 490156; 403721; 481056; 403873; 442957; 487351; 491952; 480808; 483312; 491021; 607755; 478770; 491286;
489168; 491754; 478575; 479074; 487309; 475035; 403441; 482861; 480348; 442987; 480979; 482373; 403735; 403813; 403812; 480438; 481689; 609779; 477768;
403801; 610255; 442951; 483664; 484067; 490069; 480812; 488516; 491618; 608158; 610316; 607374; 608559; 481051; 403974; 481328; 403439; 403790; 403985;
403545; 403850; 478906; 478905; 488540; 607422; 483665; 485209; 484990; 481500; 486148; 486493; 488437; 477956; 442952; 484185; 609429; 609409; 491042;
492244; 442939; 478151; 403616; 450207; 479193; 477886; 610902; 476196; 403451; 480488; 480218; 403537; 403693; 476262; 403438; 477019; 403733; 403445;
489432; 403885; 403763; 606853; 494002; 403768; 448784; 483995; 477232; 403924; 485069; 611940; 490224; 488447; 481122; 485677; 608738; 480618; 485930;
486495; 403692; 478813; 442859; 475450; 478977; 611645; 403822; 483148; 475083; 482260; 474472; 490404; 484147; 484670; 485203; 607885; 481692; 488544;
481417; 477166; 442860; 442985; 479875; 403879; 478938; 481772; 487235; 609956; 484963; 480648; 481513; 486960; 403426; 476446; 485874; 479473; 403602;
489971; 611500; 481756; 403606; 403609; 491303; 612199; 489082; 479351; 489571; 403556; 480459; 490904; 494005; 609934; 490722; 474176; 477575; 486758;
478311; 477746; 478347; 611939; 489547; 479363; 480338; 403832; 610774; 403589; 479981; 442942; 475108; 477263; 486717; 477488; 490255; 483617; 476290;
403955; 484648; 477045; 479158; 476915; 483729; 479112; 488584; 487353; 612448; 403787; 449622; 608403; 403647; 611239; 479487; 490439; 476710; 486407;
478478; 478336; 483042; 490682; 486507; 488405; 612159; 478471; 485864; 612524; 488449; 490967; 490968; 490969; 485088; 484196; 485079; 403412; 403998;
481628; 490163; 474355; 487486; 403752; 483343; 448807; 403922; 480525; 403869; 489620; 486004; 485406; 609156; 403575; 485016; 608060; 486588; 403802;
494000; 484056; 480621; 476447; 490532; 477237; 480645; 475864; 607083; 483104; 404007; 477990; 485850; 485915; 486271; 479941; 481319; 475451; 482512;
490395; 488125; 478492; 490006; 485548; 488195; 610928; 478040; 403482; 479428; 477302; 490926; 442949; 486133; 487057; 483367; 482101; 484822; 479172;
480698; 479648; 474581; 487132; 487524; 481296; 488653; 607247; 612867; 491326; 485017; 610337; 483267; 488025; 485504; 476150; 491907; 480818; 483160;
479465; 479336; 611951; 491064; 487316; 475979; 479684; 474444; 608707; 479127; 484198; 474851; 442959; 480287; 607929; 474434; 612343; 611955; 480709;
609312; 484698; 476905; 480595; 477764; 612153; 477818; 478130; 612797; 403587; 610120; 489636; 610101; 477864; 478243; 486831; 480058; 491225; 479734;
476693; 477246; 611936; 609452; 485625; 486723; 488237; 610949
rat subset 1 (i)
361523; 299626; 498545; 365458; 64639; 366960; 24482; 24483; 25685; 25285; 292152; 303698; 114851; 25405; 299626; 24330; 292041; 24329; 29200; 24517; 83681
rat subset 2 (i)
24224; 170929; 24888; 60434; 140908; 83571; 497663; 116549; 25112; 287765; 170915; 24336; 312812; 313474; 24337; 29496; 59325; 50646; 301737; 84482; 294515;
314322; 25019; 361388; 81659; 81664; 81504; 84427; 498416; 497769; 29424; 297893; 112400; 29560; 299331; 25718; 79438; 25685; 25662; 24484; 360622; 25285;
25641; 289560; 302822; 246144; 25196; 64549; 65038; 24954; 315346; 24516; 83628; 300242; 287702; 294853; 25631; 50554; 60430; 314856; 24577; 299626; 64824;
314910; 300955; 311658; 360610; 81526; 29431; 25737; 497724; 289021; 24649; 29741; 29525; 113983; 25738; 25097; 64204; 117540; 289758; 25682; 25664; 497991;
24668; 362102; 25636; 24680; 29670; 25614; 366275; 24697; 116689; 25622; 497775; 25676; 24708; 309165; 360857; 81778; 307947; 85385; 171379; 29219; 292688;
84471; 291464; 313845; 85384; 24790; 25353; 83805; 25124; 25125; 24918; 25126; 24827; 29610; 292994; 24842; 362675; 292925; 25156; 296603; 29169; 56011; 29753;
25576; 25578; 313666; 309072; 25445; 360908; 316369; 58970; 362045; 252971; 360725; 306886; 500989; 295052; 360640; 89829; 290361; 83422; 312849; 114552;
303882; 114031; 295378; 291005; 25577; 317366; 292763; 293476; 292699; 296639; 308854; 114858; 361666; 362738; 288916; 116674
rat subset 3 (i)
24153; 311860; 29437; 79558; 29381; 29263; 25366; 25237; 29650; 25591; 24176; 25238; 308578; 81637; 140670; 81638; 24185; 25378; 25673; 78963; 79116; 246097;
25385; 24208; 29183; 79120; 117273; 64373; 315305; 25389; 300711; 64639; 116502; 24887; 58919; 24224; 170929; 24888; 60434; 303836; 25181; 64625; 114496;
309871; 29373; 85272; 81507; 367786; 84480; 140923; 497672; 114486; 64022; 24242; 50663; 24244; 25400; 24245; 171140; 81715; 25166; 25402; 24247; 24248; 25404;
29161; 367218; 156726; 500985; 171136; 25706; 114839; 25729; 29157; 60350; 25073; 171369; 84349; 25406; 29364; 54237; 171102; 83502; 497718; 94201; 140908;
114483; 362110; 114851; 83571; 246060; 25164; 54238; 289993; 66019; 24252; 24253; 24258; 24854; 117027; 29393; 25412; 84032; 497663; 54245; 25419; 81649;
78965; 315707; 116549; 64032; 24962; 84353; 64529; 24296; 293985; 29277; 29296; 25086; 171352; 24307; 29139; 24310; 25112; 287765; 296500; 312477; 25433;
399489; 315219; 25313; 24329; 24330; 114090; 25148; 25129; 116636; 497806; 363854; 314436; 170915; 313052; 24335; 24336; 312812; 313474; 24337; 29496; 59323;
292673; 59325; 170909; 25149; 29251; 25439; 116677; 25584; 260320; 50646; 84490; 301737; 54250; 29348; 84489; 25022; 63839; 84482; 25661; 314322; 308411;
56718; 29292; 25019; 361388; 81659; 29152; 24388; 25235; 25236; 316033; 81664; 313017; 84475; 171104; 81504; 84427; 498416; 497769; 171101; 24938; 84027;
29424; 297893; 84577; 24446; 112400; 29560; 24451; 117282; 368057; 502373; 24448; 25461; 299331; 25261; 25700; 360415; 25712; 24482; 25718; 24483; 25151;
79438; 25685; 25662; 24484; 360622; 25285; 25641; 289560; 302822; 24494; 25663; 24497; 24498; 25205; 54258; 29385; 24504; 25196; 64549; 24506; 65038; 24954;
24508; 25467; 315346; 296456; 24511; 84598; 24514; 24516; 24518; 83628; 292855; 24917; 24525; 50687; 192362; 313050; 300438; 25608; 24536; 116641; 81515;
25631; 50554; 25750; 291439; 60430; 314856; 308106; 497834; 81686; 171045; 25335; 81687; 171052; 304608; 81709; 498982; 296344; 24577; 299626; 337868; 29148;
64824; 314910; 300955; 24592; 311658; 366998; 360610; 83619; 81736; 25336; 289222; 314969; 24599; 29492; 81526; 312195; 59109; 25054; 25342; 29597; 29431;
313262; 114502; 500242; 25737; 25267; 497724; 81745; 289021; 24649; 29741; 298696; 29525; 113983; 297804; 25619; 25738; 25097; 85253; 25515; 117540; 25682;
25664; 24668; 362102; 65179; 29471; 65248; 78975; 25636; 24680; 170538; 29340; 81749; 360748; 116590; 50689; 170851; 58960; 303200; 24684; 314615; 29670;
50557; 25637; 84023; 25652; 29527; 25614; 366275; 24697; 116689; 25622; 24699; 497775; 363875; 24703; 24706; 25676; 24708; 309165; 89809; 84583; 360857; 81771;
117269; 24615; 24772; 307947; 29517; 116743; 24775; 85385; 171379; 361825; 29219; 84471; 313845; 85384; 24790; 367846; 24791; 25353; 83805; 361295; 25124;
25125; 24918; 25126; 314621; 25155; 57027; 171046; 301985; 24827; 59086; 29591; 29610; 83580; 292994; 310553; 24835; 24842; 362675; 84494; 292925; 85489;
25708; 25156; 296603; 24873; 83785; 24874; 54319; 29169; 84495; 24884; 56011; 29753; 25578; 313666; 78947; 29448; 498798; 309072; 84584; 310678; 310838;
316369; 307766; 60664; 58970; 246334; 362045; 252971; 29376; 306886; 364420; 29576; 84578; 84385; 113894; 500989; 295052; 360640; 89829; 290361; 362485;
56084; 300057; 29455; 29521; 59103; 25414; 500616; 291309; 299694; 65035; 114552; 64547; 362111; 65169; 297508; 303882; 306141; 311030; 114031; 117043; 83427;
83614; 363014; 24373; 303477; 117268; 362160; 83726; 314850; 64537; 83516; 291005; 361798; 317366; 314126; 363140; 84016; 292699; 314612; 296639; 308854;
312574; 361057; 114512; 27064; 64031; 361374; 84357; 59301; 292041; 114858; 313729; 361666; 311299; 368128; 296551; 288916; 171437; 305828; 116674; 25528;
301045; 171018; 364681; 362535
mouse subset 1 (i)
mouse subset 2 (i)
12043; 12047; 12048; 12050; 12568; 12576; 12914; 12995; 13001; 13197; 13207; 328572; 13857; 13860; 13858; 13866; 13867; 13874; 13982; 19229; 14158; 56458;
56484; 14281; 14375; 14388; 231580; 14678; 14784; 14786; 14783; 50915; 15194; 433759; 211323; 15251; 15519; 21923; 16001; 16005; 16006; 16008; 16009; 16010;
16011; 16012; 29817; 209268; 16185; 16188; 16324; 16325; 16332; 16337; 16402; 16476; 12521; 110310; 16691; 16667; 16668; 17127; 17128; 17210; 17246; 17342;
54601; 17829; 17869; 17873; 17936; 17937; 17973; 18019; 18023; 18133; 18479; 18538; 18596; 240752; 18712; 18708; 18709; 18738; 18740; 18803; 18806; 18810;
433328; 18972; 19015; 19016; 19014; 19045; 110854; 18747; 19084; 18750; 26419; 19167; 14083; 20459; 19246; 15170; 19247; 19271; 218397; 19645; 19697; 19734;
20194; 56086; 20416; 20586; 20617; 107686; 56440; 67804; 20662; 20663; 20683; 20750; 20779; 20846; 20848; 20850; 20851; 22041; 21802; 21814; 21825; 21872;
619653; 22059; 22062; 22088; 546265; 22324; 22325; 22370; 54401; 22627; 22629; 22631; 22642; 11489; 14283; 12545; 17974; 13244; 12703; 69150; 19766; 22687;
12427; 53859; 12702; 13449; 17344; 108098; 12049; 51789; 83554; 57257; 23882; 22630; 18715; 26411; 74204; 80794; 68379; 69121; 20418; 66165; 72183; 78829;
102060; 319161; 81703
mouse subset 3 (i)
232345; 11350; 11459; 11477; 11479; 11480; 11481; 11482; 11487; 11545; 11555; 110355; 11771; 11772; 71770; 11608; 11651; 11705; 11747; 11783; 11792; 11799;
14102; 14103; 11835; 11839; 11843; 11848; 11852; 11908; 11910; 11920; 12015; 12018; 12028; 12443; 12043; 12047; 12048; 12050; 12053; 12111; 12122; 12124; 12142;
12156; 12162; 12166; 12169; 624862; 12177; 12189; 12223; 12313; 12314; 12315; 12322; 12323; 12325; 12328; 12362; 12367; 12374; 12359; 12389; 12391; 12393;
12399; 12402; 208650; 12426; 51813; 12445; 12447; 12452; 12475; 20778; 21939; 21947; 12505; 16423; 12534; 12530; 12550; 12558; 12567; 12568; 12571; 107951;
12575; 12576; 12577; 12580; 12581; 72391; 12591; 12606; 12608; 12652; 12759; 12771; 12842; 12824; 12825; 12914; 12928; 12944; 26416; 12977; 12988; 12995; 13001;
14219; 107869; 12387; 13030; 13076; 72303; 72082; 13098; 13106; 13113; 13120; 13179; 11421; 13197; 13207; 13390; 13448; 15200; 13555; 72962; 13645; 13649;
13653; 13654; 13655; 13656; 13685; 15569; 15568; 13712; 328572; 269587; 13856; 13857; 13860; 13858; 13866; 13867; 13869; 13870; 13874; 13876; 13982; 13983;
14061; 14062; 14063; 14066; 14068; 19229; 14156; 14158; 14173; 14178; 14182; 14184; 14183; 14198; 14200; 56458; 14268; 14281; 14282; 14284; 56717; 14337; 14375;
14388; 231580; 17700; 14581; 14600; 14734; 12091; 14678; 55948; 12766; 76854; 14784; 14786; 14783; 50915; 107746; 14799; 225642; 14829; 56637; 15194; 433759;
15182; 15234; 211323; 15251; 15368; 15387; 15395; 15461; 15465; 15466; 15519; 21923; 15901; 15925; 52668; 15978; 16000; 16001; 16002; 16004; 16005; 16006;
16008; 16009; 16010; 16011; 16012; 29817; 209268; 16176; 16177; 16185; 16191; 16193; 16195; 227288; 12765; 16322; 16324; 16325; 16334; 16332; 16337; 16362;
16367; 16402; 16410; 16412; 16451; 16452; 16476; 16478; 12521; 18050; 16211; 16653; 16728; 16782; 16818; 16835; 16846; 16847; 56048; 17096; 17127; 17128;
109731; 17190; 17210; 17246; 26407; 17295; 17350; 17390; 17392; 17393; 17395; 17386; 58223; 17685; 17750; 17829; 17863; 17865; 17869; 17873; 17927; 17928;
17936; 17937; 17973; 18015; 18019; 18022; 18023; 18024; 18033; 18045; 27045; 110109; 18126; 18129; 18133; 18181; 18211; 18212; 18430; 18442; 18479; 18491;
18505; 23983; 18538; 18595; 18596; 18607; 18671; 240752; 18712; 18708; 18709; 23988; 18738; 18740; 56711; 18792; 18793; 18803; 18806; 18815; 18817; 433328;
19015; 19016; 19045; 110854; 19060; 19065; 105787; 108079; 18747; 19084; 18750; 18753; 18754; 18755; 19090; 26413; 26417; 50772; 26419; 26395; 26396; 26397;
19116; 19152; 19167; 19211; 19216; 19219; 19220; 19225; 14083; 20459; 19246; 15170; 19247; 19264; 19271; 19272; 19353; 110157; 218772; 218397; 19645; 19697;
19687; 19735; 19734; 20111; 20112; 20198; 20315; 56086; 20393; 20404; 20415; 20416; 20586; 20587; 20617; 56440; 20662; 20663; 20683; 20687; 20692; 20750; 20779;
625480; 268996; 20846; 20848; 20850; 20851; 20869; 20963; 11491; 21423; 21752; 21802; 21803; 21812; 21814; 21824; 21825; 21872; 619653; 24088; 21926; 21973;
22059; 22062; 22066; 22088; 22160; 546265; 22229; 22324; 22325; 22337; 22339; 22346; 22350; 22370; 22594; 22612; 54401; 22627; 22631; 22642; 57377; 19349;
20844; 11489; 14563; 17979; 382523; 72399; 29815; 17974; 93762; 18710; 13244; 22061; 12703; 384783; 19766; 21933; 22403; 15183; 18171; 18412; 22687; 12427;
53859; 12702; 13449; 12448; 15239; 79456; 23886; 107767; 64292; 12927; 56468; 98910; 329504; 77976; 12355; 11308; 12049; 12125; 27387; 24045; 59027; 51789;
24064; 56324; 83554; 245670; 14694; 229615; 22601; 14313; 140486; 20218; 13046; 13018; 327826; 15442; 19017; 23882; 110147; 18715; 217578; 56289; 74053; 80794;
216148; 68379; 69121; 16206; 18130; 56321; 107765; 170770; 18569; 26992; 58194; 58991; 80707; 20418; 74155; 66165; 74268; 545291; 23850; 107733; 78829; 102060;
76233; 319161; 67296; 81703; 19261; 70031; 269800; 218215; 52829
dog subset 1 (l)
480897; 488577; 478327; 608843; 475864; 479849; 479203; 609301; 476882; 478858; 477858; 494010; 476467; 477060; 476576; 607923; 486962; 492057; 474652; 481587; 475535; 494010; 478249; 480558;
403878; 477076; 403647; 475472; 475535; 403589; 482506; 482506; 476261; 612603; 489373; 484765; 486962; 478813; 488559; 481587; 474652
dog subset 2 (l)
403416; 475474; 490792; 488002; 475124; 478906; 478905; 609429; 477024; 442859; 486858; 488958; 611104; 480424; 483955; 403606; 484648; 476915; 483729;
478328; 484164; 611304; 480141; 608403; 485940; 611193; 487731; 403634; 487437; 403869; 480673; 480645; 489818; 475864; 489897; 477480; 609233; 485548;
611262; 403498; 490263; 489162; 479884; 475756; 610191; 485657; 609509; 479648; 611928; 607662; 476713; 486638; 475224; 487359; 612549; 475829; 612902;
489453; 607149; 612152; 476189; 480806; 481255; 487367; 485823; 482895; 486722; 482286; 477526; 481682; 483699; 479682; 483189; 486683; 491183; 488433
dog subset 3 (l)
477699; 483331; 403580; 477236; 486888; 611658; 488793; 403783; 490878; 482558; 485189; 474503; 479797; 403579; 487718; 483763; 403416; 607482; 475526;
476694; 612941; 403567; 608401; 483134; 489369; 483845; 485338; 403765; 484780; 485790; 610456; 403934; 442858; 489775; 475537; 403429; 403541; 491011;
403651; 487948; 478806; 479866; 403856; 403923; 481276; 479331; 477032; 483662; 608543; 482665; 403400; 483038; 479740; 415122; 415120; 415128; 479740;
415127; 482506; 608452; 477807; 610489; 610935; 491319; 492250; 403424; 404306; 481528; 482613; 487380; 403883; 403640; 403639; 474468; 610360; 475473;
475474; 477295; 403845; 490792; 490252; 609642; 403755; 490520; 479885; 403418; 609246; 480708; 487351; 608234; 478047; 478575; 479911; 613006; 488002;
475597; 476731; 609164; 487207; 490630; 488266; 480110; 486068; 608119; 479182; 442987; 490657; 485742; 484434; 485884; 611913; 478450; 608958; 607052;
476411; 449023; 475124; 403975; 442951; 488516; 403974; 403985; 403850; 611642; 403628; 475574; 483665; 403937; 481500; 475633; 476412; 482264; 480015;
486148; 486493; 477956; 481746; 609429; 484927; 609409; 403758; 487743; 478666; 608024; 477601; 477675; 489800; 480488; 403693; 480481; 476215; 491946;
489432; 403924; 482978; 477024; 480646; 611940; 607700; 481277; 608738; 480021; 483925; 485930; 479678; 480272; 486495; 403692; 442859; 486858; 488958;
480559; 611104; 403822; 482260; 491866; 484670; 475375; 476975; 480424; 477166; 485757; 488573; 403624; 476828; 479875; 480011; 403879; 483955; 483266;
611266; 490013; 403659; 403606; 478980; 481945; 479351; 490904; 609091; 489532; 477746; 607019; 478464; 611939; 490291; 403832; 610774; 403797; 479981;
442942; 475108; 403662; 484648; 488774; 476915; 483729; 478328; 484164; 611304; 403615; 480141; 607023; 403650; 489972; 489973; 489508; 608403; 490439;
403748; 482437; 492299; 487272; 491184; 486908; 484068; 403655; 486507; 612159; 611255; 482182; 485940; 484403; 403815; 491950; 480815; 611193; 482771;
489736; 403444; 487731; 488605; 403922; 484006; 403869; 489620; 611956; 480673; 483876; 479883; 474536; 403574; 403576; 403575; 475378; 403802; 477991;
477237; 480645; 489818; 483104; 489897; 403534; 610421; 477392; 609418; 485548; 610928; 477052; 607042; 609634; 403498; 490263; 475660; 489162; 479884;
479940; 486973; 475756; 485657; 483640; 609509; 479648; 607662; 476713; 483681; 612928; 474494; 608092; 612867; 486638; 610835; 487359; 482049; 612549;
490376; 475930; 608155; 475829; 612902; 489453; 481233; 609003; 608707; 479127; 610407; 607060; 477901; 476617; 607149; 490685; 488673; 612152; 476189;
486981; 481255; 611920; 487367; 488970; 610914; 482895; 403598; 486722; 491743; 612831; 482286; 489412; 481682; 482744; 490273; 609390; 483189; 610156;
486683; 478879
rat subset 1 (l)
rat subset 2 (l)
24224; 24367; 314322; 303918; 54258; 29385; 24516; 301059; 81736; 309452; 25494; 85269; 65051; 304690; 25664; 24703; 24708; 309165; 300807; 309288; 83521; 295214; 307947; 261730; 117526; 304109;
25625; 497808; 24842; 311786; 311245; 29753; 56010; 25576; 25578; 246760; 498183; 58945; 29376; 246756; 310635; 363603; 116501; 29702; 84591; 117186; 292173; 25414; 207119; 363287; 117519;
25560; 298545; 117514; 113886; 315333; 302983; 360773; 366960; 83718; 366232; 306492; 312710; 304580; 64027; 365396; 498944; 310664; 297682; 287474; 303342; 311151
rat subset 3 (l)
24153; 25120; 81822; 24165; 24174; 24175; 24179; 24185; 79116; 25387; 79255; 24221; 304127; 64639; 24224; 114486; 29618; 64202; 25400; 25166; 25402; 29209;
500985; 114494; 25408; 25406; 171102; 171103; 307511; 64465; 83502; 307618; 140908; 83571; 171054; 287673; 29393; 304024; 497663; 81649; 116630; 497822;
113927; 84353; 171293; 290257; 50654; 24296; 171352; 246070; 293985; 29277; 29296; 171522; 25086; 171352; 286929; 50549; 24307; 25146; 301517; 24314; 140694;
363522; 24323; 24329; 24330; 362871; 170915; 313633; 24337; 25149; 304929; 315191; 361338; 361969; 24367; 84482; 294515; 25661; 314322; 60349; 25319; 83826;
24383; 29300; 27100; 24392; 64045; 64528; 313017; 24413; 114105; 81503; 84027; 64352; 83830; 303918; 306516; 316123; 81674; 266708; 171528; 24438; 117056;
29395; 360704; 25675; 24451; 25098; 25099; 25100; 25735; 65194; 79256; 287647; 368178; 60421; 25116; 25464; 25718; 25662; 24494; 24498; 24500; 25325; 66029;
24506; 365382; 24508; 290749; 292892; 296953; 364081; 25467; 315346; 24511; 25679; 24516; 24517; 24518; 83628; 65195; 25471; 289023; 288001; 85245; 294853;
24534; 65172; 314856; 303604; 365057; 25335; 24577; 298894; 301059; 65261; 337868; 114553; 500904; 307231; 311658; 361400; 305897; 366998; 360610; 81736;
309452; 25494; 83782; 85269; 24599; 312195; 302552; 25342; 117254; 65051; 25737; 25121; 289019; 25343; 116551; 81745; 24640; 304690; 298947; 287527; 302915;
24664; 25664; 289219; 83471; 65248; 24680; 85421; 114509; 25272; 58960; 305066; 50557; 25637; 81752; 24929; 25652; 29527; 25614; 24703; 192213; 24708; 309165;
54293; 300807; 309288; 83521; 498783; 295214; 60352; 25122; 24767; 24768; 307947; 85385; 29483; 366432; 24778; 83511; 25553; 25549; 24904; 29503; 24790; 24791;
366126; 360268; 261730; 292915; 24807; 317256; 373541; 117526; 24886; 361403; 25085; 304109; 289337; 24835; 24839; 24842; 362675; 24848; 311786; 311245;
60381; 24855; 58819; 24860; 54315; 25708; 29421; 83785; 81818; 56011; 29753; 56010; 25576; 192675; 78947; 246760; 25379; 301555; 117516; 29376; 29576; 301164;
296566; 116642; 310635; 298914; 363603; 116501; 310833; 25429; 29702; 117186; 116457; 292173; 25414; 363287; 117519; 170582; 60351; 300084; 497768; 64547;
25560; 171497; 170842; 298545; 299923; 64619; 373542; 296787; 50551; 117514; 113886; 315159; 360161; 64537; 83516; 192269; 25577; 312916; 252919; 315333;
503027; 63995; 302983; 366960; 293668; 83718; 307940; 171411; 306492; 56817; 312710; 317468; 59268; 171554; 315650; 64027; 287005; 116469; 171358; 171114;
310664; 361882; 363273; 297682; 287474; 499405
mouse subset 1 (l)
18555; 13034; 13038; 22631; 21959; 71869; 53424; 18429; 16828; 227120; 20708; 14373; 18242; 13077; 51798; 56794; 13421; 15360; 13122; 14605; 26420; 18391; 434264; 11997; 404195; 66058; 13077;
20394; 18102; 11938; 330122; 66904; 20393; 110135; 66058; 19219; 624900; 624900; 20519; 17117; 15288; 27399; 22166; 54123; 13122; 269346; 57248; 546643; 57248; 18024; 12227; 18391; 404195;
434264; 11997; 26420
mouse subset 2 (l)
12043; 99571; 14281; 74197; 15499; 227288; 12765; 16476; 17874; 18033; 18034; 18073; 79059; 268591; 241196; 19016; 110157; 19645; 19697; 19883; 225998; 56437; 20193; 394252; 56086; 20878;
228980; 21374; 21844; 21937; 21938; 22059; 22030; 22034; 22627; 22628; 22629; 22631; 17135; 17165; 56455; 384783; 71609; 16800; 17164; 69125; 65962; 13666; 18693; 24056; 237336; 12927;
225870; 208727; 50868; 20928; 100986; 194231; 56338; 26405; 16562; 223922; 71828; 67037; 30957; 83814; 77053; 17133; 29876; 19700; 18983; 26894; 11496; 231637; 192120; 245857; 72236;
229589; 70686; 52700; 237860; 68082
mouse subset 3 (l)
232345; 11298; 11461; 11486; 11551; 11552; 11553; 11606; 11651; 11792; 109689; 11911; 11937; 11998; 12013; 12015; 12043; 110279; 12226; 12317; 12322; 12362;
12367; 12372; 12373; 12402; 12428; 12519; 12505; 12530; 12531; 12532; 12540; 12550; 12562; 12568; 12576; 12773; 12775; 12842; 12892; 11909; 12914; 26416; 12981;
12984; 93687; 12387; 13033; 13035; 229445; 13040; 13076; 13078; 13113; 13095; 72303; 72082; 13098; 56448; 13106; 13113; 337924; 624900; 13120; 13074; 104086;
18104; 13429; 69537; 66705; 13614; 13649; 13653; 13713; 328572; 13844; 13866; 13982; 13983; 14067; 14114; 14151; 14161; 99571; 56458; 56484; 14268; 14281;
26424; 14319; 26423; 622339; 14465; 11692; 14609; 93692; 99412; 55948; 14723; 14724; 14815; 20310; 14825; 56637; 14866; 83602; 74197; 68153; 98053; 68705;
14938; 14939; 107970; 15078; 627468; 15312; 15357; 15368; 15375; 15376; 15377; 15378; 13340; 225307; 15417; 15426; 15469; 15483; 15499; 15894; 16001; 16008;
16176; 16193; 16199; 16153; 15930; 16334; 212111; 16362; 16363; 54131; 27056; 54139; 16367; 16402; 16412; 16440; 16476; 16477; 16478; 12521; 56543; 16509;
280287; 16644; 16647; 16668; 16832; 16885; 17246; 26406; 26408; 54601; 17393; 17869; 18109; 17874; 17913; 17927; 17969; 17972; 18018; 18019; 18021; 73181;
18022; 18023; 18033; 18034; 18073; 18103; 79059; 18126; 18181; 209815; 18430; 18477; 110821; 268591; 18538; 18549; 18557; 20028; 228026; 18607; 18640; 18671;
241196; 30955; 18816; 54128; 18976; 19016; 19044; 19072; 105787; 18750; 18760; 23939; 26419; 19094; 26414; 26396; 19130; 19211; 19216; 19217; 19218; 19220;
19225; 14083; 19249; 110157; 19417; 19645; 19697; 50780; 19883; 225998; 56437; 20191; 20193; 29857; 394252; 20276; 20277; 20278; 26398; 56086; 20416; 20512;
242607; 20525; 20537; 20538; 15567; 214084; 20517; 20518; 20683; 20692; 20375; 20860; 20878; 54200; 21336; 270627; 228980; 545600; 21374; 21391; 21750; 21823;
21844; 53791; 21926; 21929; 21958; 22059; 22062; 21990; 22030; 22034; 22099; 22084; 50493; 22227; 22228; 22229; 22275; 22339; 22352; 54401; 22627; 22628; 22629;
140475; 57377; 17135; 11737; 75533; 26554; 21943; 384783; 22403; 18519; 69454; 434203; 16800; 17164; 16413; 69125; 65962; 214137; 13123; 13666; 24056; 19099;
237336; 12927; 208727; 50868; 14170; 20186; 56438; 21353; 12125; 20928; 27219; 22057; 194231; 17988; 30928; 59041; 20348; 26462; 56338; 26405; 16562; 57259;
59126; 15442; 19017; 20024; 22630; 26754; 76257; 223922; 71828; 77057; 399510; 63953; 67037; 30957; 83814; 170755; 17133; 107392; 29876; 244667; 65963; 18983;
80906; 26894; 170743; 76500; 11496; 56057; 192120; 66259; 194352; 215303; 29811; 229589; 110596; 26895; 70686; 52700; 271424; 72823

TABLE 6A
primate toxicology biomarkers

human subset 1 (j)

| | | |
|---|---|---|
| 56140 | PCDHA8 | protocadherin alpha 8 |
| 4297 | MLL | myeloid/lymphoid or mixed-lineage leukemia (trithorax homolog, Drosophila) |
| 113419 | TEX261 | testis expressed sequence 261 |
| 80157 | FLJ21511 | hypothetical protein FLJ21511 |
| 57092 | PCNP | PEST proteolytic signal containing nuclear protein |
| 57092 | PCNP | PEST proteolytic signal containing nuclear protein |
| 65109 | UPF3B | UPF3 regulator of nonsense transcripts homolog B (yeast) |
| 5373 | PMM2 | phosphomannomutase 2 |
| 79651 | RHBDF2 | rhomboid 5 homolog 2 (Drosophila) |
| 6224 | RPS20 | ribosomal protein S20 |
| 206358 | SLC36A1 | solute carrier family 36 (proton/amino acid symporter), member 1 |
| 5300 | PIN1 | protein (peptidylprolyl cis/trans isomerase) NIMA-interacting 1 |
| 84067 | C11orf56 | chromosome 11 open reading frame 56 |
| 397 | ARHGDIB | Rho GDP dissociation inhibitor (GDI) beta |
| 11313 | LYPLA2 | lysophospholipase II |
| 29766 | TMOD3 | tropomodulin 3 (ubiquitous) |
| 55892 | MYNN | myoneurin |
| 246 | ALOX15 | arachidonate 15-lipoxygenase |
| 54870 | QRICH1 | glutamine-rich 1 |

human subset 2 (j)
399; 998; 5347; 5364; 5430; 5770; 5867; 5879; 5880; 5894; 5929; 6045; 6829; 6880; 7153; 7157; 7161; 7409; 7430; 9088; 9585; 10200; 10450; 11091; 23645; 51678; 84148; 115426; 140890
human subset 3 (j)
239; 240; 243; 245; 330; 356; 387; 396; 398; 399; 595; 596; 648; 836; 867; 983; 995; 996; 998; 1025; 1026; 1387; 1457; 1459; 1460; 1487; 1499; 1601; 1728; 2033; 2322;
2353; 2547; 2664; 2665; 2885; 3065; 3066; 3204; 3205; 3224; 3265; 3479; 3596; 3725; 3899; 4137; 4211; 4221; 4298; 4299; 4300; 4303; 4609; 4686; 4734; 4773; 5301;
5302; 5364; 5372; 5374; 5430; 5580; 5594; 5743; 5867; 5879; 5880; 5894; 5929; 5962; 5970; 5976; 6045; 6446; 6455; 6457; 6477; 6478; 6550; 6655; 6772; 6774; 6829;
6885; 7013; 7044; 7111; 7153; 7157; 7161; 7297; 7409; 7430; 8028; 8178; 8202; 8289; 8301; 9070; 9088; 9585; 9939; 10006; 10243; 10434; 10450; 10482; 10509; 10801;
10921; 11091; 11200; 11252; 22916; 23048; 23157; 23645; 23659; 26019; 29124; 29767; 29929; 51096; 55617; 57492; 57667; 65110; 80312; 84148; 115426; 152503;
153201; 285641; 389168
chimpanzee subset 1 (j)
471652; 451587; 459307; 461336; 460559; 460559; 465954; 453905; 454905; 464184; 471708; 456622; 467683; 460363
chimpanzee subset 2 (j)
471176; 456609; 453991; 460344; 469970; 463253; 460182; 457581; 455647; 467880; 450592; 454270; 456784; 456191; 472317; 473164
chimpanzee subset 3 (j)
455242; 451507; 469584; 471176; 466023; 451690; 450481; 454815; 456609; 467891; 461905; 468012; 458862; 452508; 453047; 458868; 450155; 450283; 464259; 462950; 466302; 456893; 470450; 469111; 461362;
473659; 464601; 467689; 469972; 460344; 458867; 463253; 460182; 466776; 466665; 456373; 457581; 463011; 453602; 465153; 460781; 452897; 470721; 462888; 472793; 457780; 464599; 455703; 455647;
458308; 456665; 451725; 464115; 467880; 450592; 457983; 452976; 456784; 451267; 453646; 453832; 458733; 470231; 460966; 465827; 456191; 454188; 450304; 469081; 467682; 456906; 452689; 450497;
473164; 461537; 462200
human subset 1 (cd)

| | | |
|---|---|---|
| 4501 | MT1X | metallothionein 1X |
| 4878 | NPPA | natriuretic peptide precursor A |
| 3162 | HMOX1 | heme oxygenase (decycling) 1 |
| 6279 | S100A8 | S100 calcium binding protein A8 (calgranulin A) |
| 8829 | NRP1 | neuropilin 1 |
| 3672 | ITGA1 | integrin, alpha 1 |
| 894 | CCND2 | cyclin D2 |
| 2885 | GRB2 | growth factor receptor-bound protein 2 |
| 1915 | EEF1A1 | eukaryotic translation elongation factor 1 alpha 1 |
| 1938 | EEF2 | eukaryotic translation elongation factor 2 |
| 713 | C1QB | complement component 1, q subcomponent, B chain |
| 1739 | DLG1 | discs, large homolog 1 (Drosophila) |
| 963 | CD53 | CD53 molecule |
| 3459 | IFNGR1 | interferon gamma receptor 1 |

human subset 2 (cd)
714; 715; 1019; 1020; 1021; 1667; 1740; 1741; 2035; 2066; 2678; 2886; 2889; 2890; 2898; 2903; 2977; 3064; 3183; 3265; 3273; 3460; 3558; 3560; 3565; 3635; 3655; 3667;
3674; 3688; 3691; 3702; 3716; 3717; 3739; 3759; 3766; 3768; 3791; 3815; 3932; 4036; 4130; 4133; 4146; 4233; 4486; 4582; 4646; 4842; 4881; 4883; 4914; 4952; 5058;
5144; 5148; 5159; 5287; 5295; 5296; 5335; 5573; 5621; 5716; 5747; 5770; 5771; 5777; 5781; 5782; 5786; 5788; 5791; 5806; 5913; 5921; 5979; 6280; 6300; 6352; 6402;
6442; 6452; 6461; 6464; 6640; 6654; 6655; 6714; 6776; 6850; 6853; 6868; 6885; 7010; 7094; 7132; 7157; 7178; 7275; 7407; 7409; 7410; 7430; 7453; 7454; 7462; 7881;
7916; 8038; 8471; 8573; 8626; 8631; 8651; 8660; 8717; 8751; 8871; 8882; 8936; 8976; 9101; 9112; 9185; 9228; 9229; 9419; 9495; 9564; 9611; 9612; 9615; 9649; 9712;
9846; 9988; 10019; 10040; 10163; 10188; 10252; 10253; 10270; 10371; 10451; 10461; 10657; 10817; 10818; 10825; 11184; 22803; 22839; 23095; 23129; 23303; 23607;
25970; 26191; 27036; 27040; 27240; 29760; 29950; 30011; 30837; 51517; 53358; 54512; 54822; 54900; 54910; 54938; 55824; 55872; 56654; 57504; 57510; 57596; 58480;
65993; 80306; 84962; 85461; 92799; 93643; 139716; 166647; 202559; 259215; 284217
human subset 3 (cd)
25; 58; 71; 88; 100; 133; 154; 156; 207; 210; 211; 240; 274; 302; 351; 356; 374; 432; 491; 493; 558; 571; 595; 597; 604; 613; 644; 671; 712; 714; 715; 721; 730; 800; 815;
847; 867; 868; 896; 898; 911; 912; 914; 928; 931; 933; 940; 951; 960; 967; 971; 999; 1003; 1019; 1020; 1021; 1026; 1027; 1028; 1029; 1032; 1084; 1231; 1280; 1281; 1288;
1293; 1295; 1305; 1385; 1386; 1392; 1398; 1399; 1432; 1436; 1441; 1442; 1445; 1457; 1495; 1499; 1535; 1571; 1601; 1605; 1615; 1639; 1667; 1668; 1730; 1740; 1741;
1742; 1759; 1814; 1840; 1843; 1933; 1936; 1950; 1956; 1969; 1978; 2017; 2035; 2052; 2057; 2064; 2066; 2185; 2260; 2261; 2263; 2308; 2309; 2321; 2322; 2324; 2332;
2475; 2534; 2549; 2678; 2852; 2886; 2888; 2890; 2898; 2901; 2903; 2977; 2983; 3064; 3091; 3163; 3172; 3183; 3265; 3273; 3303; 3329; 3336; 3339; 3439; 3458; 3460;
3480; 3558; 3565; 3566; 3567; 3572; 3576; 3578; 3594; 3596; 3617; 3620; 3635; 3643; 3655; 3667; 3673; 3674; 3675; 3676; 3678; 3683; 3688; 3690; 3691; 3702; 3716;
3717; 3725; 3739; 3741; 3759; 3766; 3768; 3791; 3815; 3845; 3932; 3937; 3952; 4036; 4063; 4125; 4130; 4133; 4146; 4155; 4233; 4297; 4486; 4489; 4493; 4494; 4495;
4496; 4499; 4500; 4502; 4582; 4609; 4646; 4687; 4688; 4690; 4771; 4780; 4790; 4842; 4879; 4881; 4883; 4914; 4915; 4952; 5058; 5105; 5148; 5156; 5159; 5166; 5174;
5228; 5287; 5295; 5296; 5335; 5336; 5361; 5494; 5566; 5573; 5580; 5594; 5595; 5599; 5716; 5728; 5747; 5770; 5771; 5777; 5781; 5782; 5786; 5788; 5791; 5792; 5806;
5868; 5877; 5894; 5898; 5899; 5915; 5921; 5925; 5979; 6273; 6274; 6278; 6280; 6282; 6283; 6300; 6347; 6348; 6352; 6402; 6405; 6442; 6452; 6461; 6464; 6513; 6517;
6640; 6654; 6655; 6667; 6670; 6714; 6772; 6776; 6833; 6850; 6853; 6868; 7010; 7015; 7040; 7041; 7057; 7094; 7124; 7132; 7157; 7178; 7352; 7407; 7409; 7410; 7422;
7423; 7430; 7454; 7456; 7462; 7535; 7827; 7869; 7881; 8290; 8411; 8471; 8573; 8626; 8631; 8660; 8828; 8871; 8882; 8936; 8976; 9101; 9112; 9185; 9402; 9495; 9564;
9611; 9612; 9615; 9649; 9712; 9787; 9846; 9988; 10019; 10096; 10097; 10163; 10188; 10252; 10253; 10254; 10270; 10371; 10461; 10512; 10575; 10657; 10699; 10817;
10818; 10825; 10928; 10957; 11183; 11184; 11186; 11234; 11252; 23048; 23139; 23303; 25960; 26191; 27036; 27040; 27240; 29760; 29904; 29950; 30011; 30837; 51455;
51517; 53358; 54822; 54900; 54910; 54938; 55103; 55243; 55872; 57504; 57510; 57596; 58480; 65993; 80306; 84623; 84962; 85320; 85476; 92304; 93643; 122011;
166647; 202559; 259215; 284217; 344148
chimpanzee subset 1 (cd)
469801; 470195; 457319; 450397; 472057; 469214; 460968; 463949
chimpanzee subset 2 (cd)
456612; 452025; 463891; 463534; 456689; 459919; 470168; 471714; 454396; 466769; 466302; 460914; 470077; 461472; 470203; 449565; 459742; 454730; 450396;
456911; 466477; 468312; 461315; 461316; 459713; 453380; 463671; 467139; 471581; 465851; 451439; 461860; 454962; 462188; 457657; 461836; 455855; 454845;
458076; 465803; 464422; 469970; 455504; 467135; 463494; 457607; 450865; 460807; 461762; 450415; 457998; 468403; 461080; 457351; 459171; 452897; 458229;
454681; 465606; 459018; 462888; 465028; 465080; 466934; 452575; 466436; 462568; 455647; 471965; 450818; 473846; 468432; 454154; 457354; 463094; 462935;
463688; 453428; 453217; 459208; 467480; 454246; 468370; 467150; 472950; 466011; 472434; 452245; 455138; 456675; 460954; 461476; 457076; 459492; 456705;
472005; 467064; 469930; 457136; 468976; 456037; 465527; 460351; 453429; 459423; 464496; 464882; 459183; 461133; 459678; 456041; 463194; 473837
chimpanzee subset 3 (cd)
464802; 454959; 451021; 471690; 451363; 453202; 464675; 460416; 459590; 453487; 469584; 461251; 456057; 458500; 463366; 456612; 462577; 461902; 451116;
451690; 460566; 462689; 455914; 457419; 469524; 449512; 451775; 451223; 470617; 456200; 451969; 465075; 452025; 463891; 463534; 465021; 459815; 465804;
450508; 464215; 470146; 462643; 462187; 456756; 454774; 462100; 468068; 450857; 461905; 460372; 459634; 459332; 468444; 464766; 470885; 471742; 465545;
464444; 456689; 457777; 455728; 454636; 459919; 464073; 461067; 452508; 462339; 465894; 461516; 470168; 459695; 471714; 454396; 466769; 452958; 453880;
458269; 466302; 460914; 459852; 452179; 456606; 473197; 449517; 470077; 453676; 461472; 449565; 454005; 461866; 471205; 462832; 464134; 455649; 459742;
454730; 455111; 470596; 451958; 450396; 468295; 456911; 456893; 466477; 466930; 468312; 461315; 461316; 473387; 471734; 449638; 459713; 457438; 453380;
463671; 451587; 454105; 471221; 457573; 459769; 461406; 467139; 471581; 465851; 451439; 454962; 461318; 462188; 472448; 457979; 453041; 457657; 461836;
455855; 454268; 452950; 454845; 454037; 450445; 465803; 466142; 464422; 469970; 455504; 467135; 463494; 457607; 450865; 456828; 460807; 457633; 460182;
470523; 461762; 452715; 450415; 457321; 469501; 457998; 457994; 469835; 460387; 468403; 461080; 457351; 468443; 459171; 452897; 451939; 459750; 458229;
470721; 454681; 451056; 465606; 459018; 465028; 471443; 456060; 467962; 453319; 465080; 466934; 452575; 462568; 455647; 466648; 459427; 467212; 473846;
468432; 460111; 463094; 462935; 463688; 453428; 453217; 467480; 454246; 468370; 467150; 472950; 466011; 452920; 472434; 452245; 456675; 460954; 461476;
459661; 459492; 472427; 456705; 461350; 472005; 467064; 467451; 460392; 451060; 470231; 456568; 472739; 457136; 468976; 454350; 456037; 465527; 460351;
453429; 459423; 457549; 464496; 459183; 461133; 473275; 460812; 463194; 467264
human subset 1 (cpm)
human subset 2 (cpm)
351; 354; 409; 624; 637; 638; 666; 949; 972; 1045; 1173; 1215; 1270; 1277; 1278; 1326; 1445; 1462; 1499; 1504; 1506; 1508; 1511; 1536; 1758; 1839; 1956; 1990; 1991;
2035; 2064; 2066; 2099; 2153; 2158; 2159; 2185; 2192; 2199; 2200; 2201; 2209; 2219; 2247; 2260; 2263; 2264; 2266; 2289; 2309; 2534; 2719; 2770; 2776; 2822; 2885;
2934; 3115; 3240; 3309; 3320; 3339; 3350; 3371; 3481; 3486; 3551; 3554; 3556; 3559; 3561; 3576; 3581; 3593; 3595; 3596; 3597; 3630; 3635; 3663; 3665; 3667; 3675;
3676; 3678; 3681; 3683; 3684; 3688; 3689; 3694; 3695; 3716; 3717; 3718; 3817; 3827; 3838; 3915; 3921; 3932; 3949; 3958; 3959; 3976; 3977; 3998; 4015; 4018; 4035;
4036; 4043; 4050; 4052; 4055; 4059; 4067; 4069; 4082; 4099; 4133; 4137; 4147; 4160; 4192; 4224; 4225; 4237; 4296; 4316; 4318; 4478; 4485; 4609; 4637; 4653; 4687;
4688; 4692; 4734; 4771; 4790; 4792; 4793; 4794; 4803; 4811; 4856; 5008; 5054; 5068; 5104; 5155; 5196; 5264; 5267; 5275; 5294; 5295; 5296; 5310; 5327; 5328; 5329;
5335; 5336; 5340; 5345; 5360; 5366; 5422; 5443; 5549; 5552; 5580; 5587; 5588; 5591; 5594; 5595; 5621; 5624; 5627; 5644; 5657; 5699; 5747; 5770; 5777; 5781; 5788;
5825; 5829; 5868; 5879; 5886; 5887; 5921; 5925; 5947; 5970; 6128; 6223; 6281; 6284; 6317; 6318; 6347; 6348; 6349; 6351; 6352; 6354; 6355; 6356; 6357; 6358; 6360;
6376; 6383; 6386; 6387; 6401; 6402; 6404; 6407; 6423; 6461; 6464; 6550; 6643; 6645; 6678; 6690; 6696; 6714; 6774; 6775; 6776; 6778; 6850; 6863; 6885; 6892; 7038;
7042; 7045; 7046; 7048; 7049; 7052; 7056; 7057; 7074; 7088; 7094; 7123; 7128; 7132; 7133; 7157; 7170; 7186; 7203; 7253; 7276; 7278; 7294; 7297; 7329; 7341; 7356;
7369; 7373; 7391; 7392; 7409; 7410; 7415; 7422; 7424; 7428; 7430; 7436; 7448; 7450; 7520; 7525; 7529; 7531; 7532; 7534; 7804; 7850; 7852; 7980; 8029; 8100; 8174;
8190; 8425; 8503; 8516; 8517; 8518; 8530; 8539; 8651; 8655; 8658; 8660; 8668; 8723; 8726; 8739; 8764; 8772; 8804; 8828; 8829; 8835; 8839; 8878; 8915; 8945; 8974;
8999; 9020; 9021; 9138; 9166; 9180; 9223; 9270; 9342; 9368; 9479; 9510; 9530; 9533; 9612; 9672; 9702; 9722; 9737; 9784; 9898; 9982; 10009; 10010; 10135; 10148;
10226; 10332; 10399; 10451; 10456; 10459; 10477; 10487; 10539; 10563; 10574; 10627; 10659; 10672; 10755; 10758; 10930; 10938; 10970; 10989; 11018; 11140; 11151;
11186; 11218; 11260; 22795; 22927; 22937; 22948; 23062; 23136; 23163; 23193; 23365; 25759; 25865; 26047; 26088; 26119; 27035; 27040; 27113; 29894; 29974; 30835;
50512; 51141; 51561; 51705; 51726; 53353; 54476; 55333; 55607; 55690; 55805; 55816; 56034; 56897; 56899; 57085; 57763; 58533; 59307; 60681; 63827; 64112; 64215;
64599; 79613; 79784; 80781; 84433; 84687; 85406; 85445; 90070; 91179; 92304; 114990; 116844; 117156; 133396; 147945; 149233; 154796; 164656; 201299; 284217;
353376; 414062
human subset 3 (cpm)
2; 12; 19; 20; 25; 26; 31; 38; 39; 43; 49; 52; 58; 60; 71; 81; 90; 100; 102; 118; 133; 142; 154; 155; 162; 166; 174; 175; 176; 177; 181; 183; 185; 186; 199; 207; 208; 213; 214;
241; 248; 259; 283; 284; 285; 290; 301; 302; 307; 308; 322; 325; 329; 332; 335; 336; 337; 338; 341; 344; 345; 347; 348; 350; 351; 354; 355; 356; 384; 387; 392; 401; 408;
409; 434; 445; 462; 467; 468; 472; 498; 506; 545; 552; 553; 554; 558; 567; 572; 578; 581; 590; 595; 596; 598; 599; 624; 627; 632; 633; 637; 638; 641; 642; 650; 651; 652;
654; 655; 660; 671; 672; 673; 695; 710; 712; 718; 728; 730; 731; 732; 733; 768; 773; 780; 794; 811; 816; 820; 821; 834; 835; 836; 837; 838; 841; 857; 860; 861; 871; 890;
894; 908; 911; 912; 920; 921; 925; 929; 930; 934; 939; 941; 942; 943; 945; 947; 948; 949; 950; 952; 958; 959; 960; 961; 962; 963; 965; 968; 972; 973; 974; 975; 999; 1003;
1017; 1019; 1025; 1026; 1027; 1029; 1045; 1050; 1051; 1071; 1075; 1080; 1111; 1113; 1147; 1158; 1173; 1179; 1191; 1213; 1215; 1218; 1230; 1231; 1232; 1234; 1235;
1236; 1237; 1238; 1241; 1270; 1271; 1277; 1278; 1280; 1281; 1282; 1284; 1289; 1290; 1291; 1292; 1293; 1294; 1302; 1305; 1308; 1312; 1316; 1325; 1326; 1356; 1357;
1358; 1359; 1361; 1363; 1364; 1371; 1387; 1388; 1398; 1399; 1401; 1404; 1414; 1432; 1435; 1437; 1440; 1445; 1457; 1459; 1460; 1462; 1471; 1473; 1475; 1476; 1489;
1490; 1497; 1499; 1500; 1504; 1506; 1508; 1509; 1511; 1512; 1513; 1514; 1520; 1524; 1535; 1536; 1544; 1557; 1571; 1579; 1584; 1585; 1601; 1604; 1605; 1616; 1634;
1636; 1641; 1649; 1660; 1667; 1672; 1673; 1675; 1719; 1739; 1740; 1741; 1742; 1757; 1758; 1759; 1785; 1803; 1813; 1815; 1816; 1822; 1827; 1832; 1839; 1869; 1889;
1890; 1901; 1906; 1909; 1910; 1942; 1943; 1950; 1956; 1958; 1969; 1977; 1978; 1981; 1990; 1991; 1992; 1994; 2011; 2017; 2022; 2023; 2029; 2033; 2034; 2035; 2056;
2064; 2065; 2066; 2081; 2091; 2099; 2100; 2101; 2113; 2114; 2118; 2119; 2147; 2149; 2150; 2152; 2153; 2157; 2158; 2159; 2160; 2162; 2165; 2168; 2169; 2185; 2192;
2194; 2199; 2200; 2201; 2205; 2207; 2208; 2209; 2212; 2214; 2219; 2224; 2235; 2237; 2239; 2242; 2243; 2244; 2246; 2247; 2249; 2250; 2251; 2252; 2253; 2254; 2255;
2258; 2260; 2261; 2263; 2264; 2266; 2268; 2272; 2277; 2297; 2321; 2324; 2331; 2335; 2337; 2339; 2342; 2353; 2354; 2355; 2357; 2495; 2526; 2534; 2539; 2547; 2618;
2621; 2623; 2626; 2638; 2650; 2670; 2673; 2678; 2688; 2689; 2694; 2719; 2770; 2771; 2776; 2778; 2784; 2803; 2811; 2817; 2822; 2829; 2833; 2838; 2885; 2887; 2888;
2889; 2902; 2908; 2920; 2921; 2923; 2932; 2934; 2950; 2952; 2969; 3002; 3014; 3036; 3037; 3038; 3055; 3064; 3065; 3075; 3077; 3078; 3082; 3087; 3091; 3099; 3105;
3106; 3107; 3108; 3112; 3113; 3115; 3117; 3118; 3119; 3122; 3123; 3133; 3134; 3135; 3145; 3146; 3161; 3162; 3163; 3164; 3169; 3170; 3171; 3172; 3178; 3240; 3263;
3265; 3273; 3276; 3297; 3303; 3309; 3315; 3316; 3320; 3329; 3339; 3350; 3351; 3358; 3362; 3371; 3375; 3381; 3383; 3384; 3385; 3386; 3394; 3397; 3399; 3439; 3454;
3456; 3458; 3459; 3479; 3480; 3481; 3482; 3485; 3486; 3487; 3488; 3489; 3490; 3493; 3500; 3514; 3547; 3551; 3552; 3553; 3554; 3556; 3557; 3558; 3559; 3560; 3561;
3562; 3563; 3565; 3566; 3567; 3569; 3570; 3572; 3573; 3574; 3576; 3577; 3579; 3581; 3586; 3587; 3588; 3589; 3590; 3592; 3593; 3694; 3595; 3596; 3597; 3598; 3600;
3603; 3604; 3605; 3606; 3624; 3627; 3630; 3635; 3638; 3643; 3645; 3654; 3655; 3659; 3660; 3663; 3665; 3667; 3672; 3673; 3674; 3675; 3676; 3678; 3680; 3681; 3683;
3684; 3685; 3687; 3688; 3689; 3690; 3691; 3693; 3694; 3695; 3696; 3716; 3717; 3718; 3725; 3727; 3728; 3764; 3767; 3778; 3782; 3783; 3791; 3815; 3816; 3817; 3818;
3827; 3838; 3856; 3897; 3908; 3909; 3910; 3911; 3912; 3913; 3914; 3915; 3916; 3918; 3920; 3921; 3931; 3932; 3934; 3949; 3952; 3953; 3957; 3958; 3976; 3977; 3984;
3990; 3998; 4012; 4015; 4018; 4023; 4035; 4036; 4037; 4040; 4041; 4043; 4049; 4050; 4051; 4052; 4054; 4055; 4059; 4067; 4069; 4074; 4082; 4087; 4088; 4089; 4091;
4092; 4099; 4128; 4133; 4137; 4140; 4153; 4155; 4157; 4158; 4159; 4160; 4161; 4162; 4170; 4179; 4192; 4193; 4203; 4205; 4214; 4217; 4221; 4224; 4225; 4233; 4237;
4254; 4261; 4270; 4276; 4282; 4283; 4286; 4288; 4296; 4306; 4311; 4312; 4313; 4314; 4316; 4317; 4318; 4319; 4320; 4321; 4322; 4323; 4327; 4353; 4360; 4361; 4478;
4485; 4496; 4524; 4548; 4582; 4583; 4585; 4586; 4589; 4590; 4609; 4615; 4653; 4661; 4665; 4683; 4687; 4688; 4689; 4690; 4692; 4704; 4734; 4739; 4763; 4771; 4772;
4778; 4790; 4791; 4792; 4793; 4794; 4795; 4801; 4803; 4811; 4830; 4838; 4839; 4842; 4843; 4844; 4845; 4846; 4851; 4853; 4856; 4868; 4904; 4923; 4948; 4958; 4982;
4985; 4986; 4988; 5008; 5021; 5026; 5027; 5028; 5029; 5031; 5032; 5045; 5048; 5054; 5055; 5058; 5068; 5069; 5082; 5104; 5111; 5119; 5126; 5144; 5154; 5155; 5159;
5163; 5167; 5169; 5175; 5176; 5196; 5228; 5241; 5243; 5264; 5265; 5267; 5268; 5269; 5271; 5272; 5273; 5274; 5275; 5286; 5290; 5292; 5294; 5295; 5296; 5303; 5310;
5320; 5321; 5327; 5328; 5329; 5335; 5336; 5338; 5340; 5342; 5343; 5345; 5346; 5347; 5360; 5422; 5423; 5443; 5444; 5445; 5446; 5465; 5467; 5468; 5551; 5552; 5563;
5566; 5575; 5578; 5579; 5580; 5581; 5583; 5584; 5585; 5587; 5588; 5590; 5591; 5594; 5595; 5599; 5600; 5601; 5603; 5604; 5605; 5606; 5617; 5621; 5624; 5627; 5644;
5649; 5651; 5653; 5657; 5663; 5684; 5686; 5689; 5693; 5695; 5696; 5698; 5699; 5720; 5721; 5724; 5728; 5732; 5734; 5739; 5742; 5743; 5747; 5764; 5770; 5777; 5781;
5788; 5813; 5825; 5829; 5834; 5858; 5859; 5868; 5879; 5884; 5886; 5887; 5888; 5893; 5894; 5901; 5915; 5921; 5925; 5934; 5947; 5962; 5970; 5972; 5984; 6019; 6037;
6041; 6093; 6095; 6101; 6117; 6128; 6161; 6163; 6173; 6187; 6195; 6198; 6223; 6235; 6240; 6256; 6257; 6272; 6279; 6280; 6281; 6284; 6285; 6303; 6311; 6314; 6317;
6318; 6319; 6337; 6346; 6347; 6348; 6349; 6351; 6352; 6354; 6355; 6356; 6357; 6358; 6360; 6361; 6364; 6367; 6369; 6372; 6373; 6374; 6376; 6382; 6383; 6385; 6386;
6387; 6401; 6402; 6403; 6404; 6405; 6407; 6416; 6423; 6428; 6431; 6435; 6439; 6440; 6446; 6461; 6464; 6480; 6497; 6513; 6520; 6530; 6531; 6532; 6541; 6546; 6550;
6571; 6590; 6616; 6645; 6646; 6648; 6653; 6667; 6670; 6678; 6688; 6690; 6693; 6696; 6711; 6714; 6720; 6721; 6722; 6736; 6750; 6751; 6752; 6753; 6754; 6755; 6768;
6772; 6774; 6775; 6776; 6778; 6794; 6850; 6862; 6863; 6868; 6885; 6890; 6891; 6892; 6901; 6915; 6927; 6928; 6947; 6950; 7006; 7010; 7013; 7014; 7018; 7020; 7033;
7035; 7037; 7038; 7040; 7041; 7042; 7043; 7045; 7046; 7048; 7049; 7052; 7056; 7057; 7058; 7060; 7064; 7066; 7067; 7073; 7074; 7075; 7076; 7077; 7078; 7080; 7087;
7088; 7096; 7097; 7098; 7099; 7100; 7114; 7123; 7124; 7128; 7130; 7132; 7133; 7146; 7148; 7157; 7161; 7166; 7184; 7185; 7186; 7187; 7188; 7253; 7276; 7280; 7292;
7294; 7295; 7297; 7299; 7329; 7341; 7356; 7358; 7369; 7373; 7376; 7391; 7392; 7402; 7409; 7410; 7412; 7415; 7416; 7421; 7422; 7423; 7424; 7428; 7430; 7432; 7436;
7448; 7449; 7450; 7454; 7456; 7465; 7476; 7490; 7494; 7520; 7525; 7528; 7529; 7531; 7532; 7534; 7535; 7538; 7702; 7704; 7804; 7818; 7850; 7852; 7869; 7881; 7916;
7917; 7918; 7919; 7920; 7940; 7941; 7980; 8029; 8061; 8081; 8174; 8190; 8202; 8263; 8398; 8425; 8435; 8459; 8460; 8471; 8503; 8516; 8517; 8518; 8530; 8531; 8539;
8567; 8570; 8600; 8607; 8626; 8637; 8651; 8658; 8660; 8668; 8681; 8694; 8698; 8717; 8720; 8726; 8737; 8741; 8743; 8751; 8754; 8764; 8772; 8792; 8793; 8794; 8795;
8804; 8808; 8809; 8823; 8828; 8829; 8835; 8837; 8839; 8848; 8853; 8877; 8878; 8900; 8907; 8915; 8928; 8936; 8945; 8999; 9020; 9021; 9023; 9034; 9076; 9135; 9138;
9156; 9158; 9159; 9170; 9173; 9180; 9185; 9218; 9220; 9223; 9232; 9242; 9252; 9261; 9267; 9294; 9367; 9368; 9370; 9372; 9401; 9451; 9466; 9507; 9508; 9509; 9510;
9530; 9533; 9536; 9569; 9588; 9619; 9622; 9635; 9641; 9672; 9702; 9737; 9784; 9836; 9970; 9971; 9982; 10000; 10005; 10053; 10057; 10060; 10062; 10148; 10211;
10226; 10242; 10250; 10257; 10288; 10319; 10332; 10333; 10344; 10347; 10365; 10376; 10381; 10383; 10399; 10413; 10423; 10454; 10456; 10477; 10486; 10487; 10492;
10516; 10539; 10542; 10544; 10563; 10574; 10576; 10628; 10631; 10659; 10663; 10664; 10672; 10749; 10755; 10758; 10776; 10788; 10797; 10803; 10818; 10850; 10855;
10858; 10859; 10890; 10911; 10919; 10930; 10970; 10987; 11009; 11018; 11095; 11140; 11151; 11162; 11171; 11186; 11213; 11253; 11260; 11283; 11329; 22795; 22861;
22914; 22926; 22937; 22948; 22973; 23062; 23136; 23163; 23213; 23365; 23428; 23468; 23529; 23566; 23586; 23621; 23643; 23673; 23683; 23705; 23729; 25759; 25865;
25900; 25913; 26085; 26088; 26119; 26133; 26156; 26279; 26511; 26512; 26525; 26585; 27035; 27040; 27124; 27185; 27339; 27347; 29108; 29110; 29942; 29974; 30000;
30835; 30851; 43849; 50507; 50508; 50512; 50616; 50943; 51141; 51176; 51199; 51213; 51214; 51247; 51284; 51311; 51360; 51561; 51645; 51705; 51726; 51738; 53353;
53637; 53905; 54106; 54361; 54472; 54567; 54577; 54882; 55311; 55554; 55607; 55612; 55690; 55827; 55922; 56034; 56547; 56848; 56897; 57085; 57154; 57663; 57699;
57763; 57834; 59082; 59272; 60528; 60675; 60681; 63036; 64112; 64215; 64750; 66002; 79400; 80142; 80310; 80351; 80781; 81565; 81608; 81693; 81793; 83737; 83740;
83881; 83903; 83939; 84432; 84433; 84624; 84687; 84870; 85406; 85480; 90070; 90411; 90865; 91179; 92304; 92552; 94025; 112399; 114548; 114990; 116519; 116844;
117156; 133396; 138948; 140564; 147945; 148022; 149233; 154796; 164656; 169714; 199974; 201288; 201299; 246778; 259197; 284043; 284217; 285189; 326625;
344697; 353125; 386653; 391533; 399687; 404734; 414062; 474381; 474382; 474383; 474384; 544447
chimpanzee subset 1 (cpm)
455700; 456213; 466763; 454451; 451628; 453088; 453946; 454258; 452904; 453130; 461261; 456778; 457461; 461473; 473153; 470418; 458198; 454478; 454329; 453177; 468770; 470057; 459462;
459571; 455803; 457280; 471247; 462744; 471965; 467481; 472732; 457762; 453319; 469898; 456060; 463368; 451056; 469897; 464032; 470182; 468208; 451181; 457558; 462596; 450288; 450503;
457450; 463548; 463549; 463547; 472748; 455461; 462393; 467541; 454766; 463002; 461318; 453132; 455460; 473656; 455026; 462637; 461717; 452965; 464003; 451509; 453114; 469978; 453184;
459713; 460365; 472122; 468971; 461315; 462258; 470789; 460816; 461866; 454005; 459501; 450200; 470485; 463112; 453676; 450196; 456606; 462599; 462598; 460081; 461558; 469107; 464580;
454782; 464827; 468068; 460281; 457277; 453574; 463993; 450128; 450673; 451230; 466792; 460762; 450164; 453133
chimpanzee subset 2 (cpm)
454450; 467547; 458635; 470232; 470705; 452359; 462191; 463542; 450384; 461766; 454179; 463993; 467413; 468637; 456689; 454636; 459919; 463079; 457500;
465887; 452679; 464073; 458908; 471621; 464840; 462301; 461560; 462639; 465866; 462473; 462599; 454233; 464733; 456606; 471533; 466318; 463396; 472749;
459460; 460935; 450884; 471205; 471723; 469348; 450954; 450927; 455111; 470596; 451958; 468103; 450396; 458607; 451934; 456911; 455848; 469100; 460916;
454841; 455722; 454930; 455448; 471614; 463116; 452006; 459713; 461086; 459143; 451778; 456118; 472771; 469075; 468556; 462745; 468515; 451509; 460377;
457511; 457573; 453273; 467689; 461406; 467426; 456014; 462730; 472467; 459349; 453132; 458844; 450310; 467544; 463649; 461836; 455855; 454340; 472748;
450103; 454268; 458291; 465543; 459074; 469649; 450503; 452834; 450288; 464165; 454037; 458076; 459593; 460532; 463797; 464422; 469970; 467135; 457607;
457037; 463253; 473012; 461762; 452715; 466665; 452266; 456065; 457306; 457324; 455459; 455004; 454579; 468209; 468208; 455038; 454115; 464298; 464192;
450191; 452212; 461556; 457351; 462022; 471709; 461366; 458229; 459834; 454681; 452005; 462888; 464407; 457742; 462081; 472992; 460243; 457016; 469898;
453319; 465080; 460314; 472136; 466934; 457968; 458000; 464876; 461344; 455703; 467865; 459882; 466634; 457443; 468835; 455647; 471358; 464967; 449614;
455322; 454409; 472830; 470455; 450130; 463540; 452460; 456048; 456044; 456565; 450323; 451131; 463982; 456714; 460652; 466901; 457221; 451389; 460111;
450397; 452127; 458271; 450685; 454750; 456067; 464339; 465215; 458669; 454865; 451148; 470057; 464116; 467150; 451497; 457463; 459100; 457346; 471149;
465831; 459685; 463648; 455600; 455661; 462344; 457076; 457347; 457953; 459792; 450828; 455314; 450298; 455719; 455704; 460392; 452046; 461721; 453987;
455309; 454877; 451258; 451618; 469237; 465752; 466076; 450160; 467036; 461402; 460911; 453003; 463546; 466669; 471371; 452157; 457954; 461810; 454668;
453122; 450344; 463607; 463235; 467031; 451963; 471545; 469019; 468223; 471605
chimpanzee subset 3 (cpm)
453133; 464802; 463895; 454602; 458950; 458990; 450133; 454959; 456010; 470565; 453472; 451021; 457785; 471690; 464113; 458967; 461258; 461616; 453629;
462584; 468002; 450104; 460762; 453202; 456035; 461260; 460565; 452520; 464334; 467758; 472953; 453487; 461466; 457427; 451508; 451564; 458013; 466792;
459058; 460951; 468459; 466149; 469584; 452985; 451156; 454450; 473074; 468334; 455397; 451999; 460745; 452038; 469656; 456057; 450170; 451291; 462616;
460830; 458156; 452796; 467547; 473829; 458635; 470232; 453650; 454555; 458090; 449497; 463781; 461302; 455644; 468931; 461902; 456886; 456887; 473223;
455756; 470816; 462330; 451519; 463804; 451518; 460110; 463670; 472017; 466899; 461467; 463421; 457419; 469524; 450124; 462118; 454020; 470892; 470900;
457960; 457692; 452359; 461239; 473802; 460569; 457437; 457154; 468440; 462191; 456066; 449635; 452025; 465021; 455931; 469969; 467982; 451469; 463674;
450673; 464075; 470810; 450128; 472188; 454654; 470795; 460298; 452818; 473218; 463542; 459815; 460347; 450508; 450717; 458655; 450269; 457927; 450384;
463728; 463726; 452582; 461592; 463464; 460544; 467891; 470146; 457428; 461765; 458727; 462643; 457127; 471627; 454641; 461766; 458593; 467955; 463003;
468161; 451203; 454179; 463993; 467413; 453574; 457278; 457277; 460281; 468068; 453745; 450857; 464436; 461905; 460372; 462613; 452120; 454782; 473733;
452017; 457568; 461773; 460968; 468444; 464766; 459689; 451553; 473340; 462420; 458184; 456594; 458941; 457063; 462437; 452613; 457356; 468644; 462097;
461391; 460888; 468637; 471841; 455668; 457913; 457275; 458862; 459204; 456689; 463609; 454636; 451980; 459919; 456031; 463079; 466651; 454715; 460906;
451158; 471510; 461800; 457038; 457500; 465887; 452679; 462413; 464073; 458908; 454986; 471621; 469538; 458012; 456434; 450194; 457457; 464840; 451242;
453654; 461559; 461558; 462158; 466692; 471190; 466925; 467226; 471553; 465886; 461067; 462301; 461560; 470835; 462339; 457644; 472929; 452971; 453047;
456127; 459115; 456254; 449507; 458868; 458518; 465619; 461270; 472959; 454741; 459296; 470168; 465866; 469986; 460264; 468166; 462473; 460321; 450102;
463410; 459695; 471681; 461255; 453387; 470895; 451717; 463472; 466864; 472850; 454195; 458163; 464259; 457602; 462489; 463503; 450612; 452958; 462532;
450202; 449592; 471977; 462598; 462599; 471974; 471976; 462591; 462594; 462529; 463179; 452518; 470195; 453880; 456143; 458269; 454233; 450992; 466302;
460914; 464733; 463488; 451544; 459852; 456606; 471533; 462833; 469199; 471249; 450196; 455698; 454294; 469912; 456618; 473197; 458516; 473183; 449517;
453676; 466318; 463112; 459932; 483396; 454652; 459933; 451929; 465861; 472749; 470485; 450200; 459460; 460935; 459501; 461472; 450884; 470203; 471626;
449565; 454005; 463288; 469506; 461866; 464245; 471205; 470645; 470644; 469657; 451580; 473219; 460816; 471723; 469348; 465815; 449644; 472029; 463371;
461242; 450954; 463839; 455649; 465935; 459742; 461629; 450927; 454730; 455111; 470596; 451958; 470789; 468103; 459807; 454070; 450396; 458607; 468295;
451934; 456911; 455848; 456893; 454667; 465337; 466450; 456098; 461315; 461316; 468971; 469100; 471375; 460916; 454841; 472122; 462946; 460365; 469668;
457571; 464764; 455722; 449638; 456919; 470204; 463466; 467694; 455448; 461960; 471614; 463116; 464031; 452006; 459713; 455929; 451380; 461086; 459143;
451778; 456118; 472771; 455407; 468537; 467711; 455411; 469075; 453184; 466126; 455500; 469978; 468556; 468599; 457274; 469663; 471543; 462745; 468515;
463671; 452114; 453914; 471200; 450866; 461533; 460792; 451510; 454094; 451511; 451509; 458701; 451512; 451513; 450333; 451488; 460377; 457844; 460952;
460269; 457511; 464278; 457573; 453273; 451766; 467689; 454566; 455485; 461406; 467426; 456014; 462730; 452191; 455134; 466100; 451784; 467139; 455026;
468841; 470311; 458392; 464351; 472761; 472165; 454434; 452318; 470970; 466702; 451410; 453652; 454422; 472467; 455460; 451439; 459349; 464690; 473052;
453132; 458081; 458106; 461860; 458844; 462188; 459743; 463002; 454766; 453041; 451502; 450310; 467541; 467544; 468566; 462393; 455461; 468570; 460834;
460994; 462656; 463649; 461836; 455855; 454340; 456587; 472748; 450103; 454268; 454454; 467755; 453991; 458291; 465543; 459074; 463547; 463549; 463548;
460178; 450512; 450503; 459203; 450143; 452834; 450288; 464165; 454037; 450445; 462644; 453526; 454515; 462468; 458076; 459593; 460532; 463797; 463635;
458478; 467503; 473258; 462596; 469249; 466142; 467459; 450195; 456154; 464713; 464422; 463759; 469970; 467135; 457607; 471649; 457037; 469908; 465371;
463253; 461832; 473012; 453339; 466911; 460182; 452414; 461762; 452715; 467978; 466776; 466665; 469651; 460918; 449633; 469607; 455326; 472766; 454414;
452266; 450727; 465760; 454344; 456065; 464834; 457319; 457998; 457306; 457324; 465538; 465220; 455459; 450676; 451776; 455004; 454579; 468209; 468208;
455038; 467985; 459991; 467984; 472418; 471204; 471203; 454115; 464298; 469953; 464192; 450191; 452212; 460387; 455065; 461556; 469946; 466120; 464041;
463011; 457351; 460922; 451271; 454096; 452516; 466213; 458096; 457553; 449634; 451939; 459750; 471709; 467939; 461366; 459234; 458229; 462709; 449510;
471035; 467434; 454854; 458807; 469897; 453812; 451667; 470721; 459834; 454681; 452005; 468641; 472186; 459018; 462888; 471978; 468667; 468227; 451215;
463368; 461343; 465028; 472793; 454201; 460710; 470603; 460956; 464407; 456060; 467962; 457742; 453054; 462081; 472992; 460243; 457016; 469898; 453319;
463138; 450172; 450776; 469308; 454928; 458784; 452867; 461167; 471325; 471374; 457762; 460314; 472136; 466934; 457968; 466453; 452187; 464702; 464876;
453178; 457711; 469586; 461344; 450779; 455703; 467865; 459882; 466634; 471171; 457443; 468835; 455647; 471632; 451858; 466648; 471358; 472164; 464967;
466440; 458735; 449614; 455322; 454409; 472830; 459427; 451017; 451557; 458006; 470455; 450130; 471965; 462560; 462562; 462563; 462744; 463540; 456048;
458308; 456044; 458726; 472398; 473846; 456565; 450323; 465289; 451131; 455640; 463982; 456714; 464460; 454154; 468050; 466901; 455220; 460852; 457354;
464129; 457221; 451389; 468560; 472715; 472713; 459463; 473676; 460111; 450397; 452127; 459872; 458271; 452573; 467260; 468761; 464468; 462935; 450685;
454750; 460324; 460934; 455255; 456067; 457856; 451571; 455878; 459462; 465215; 462238; 472790; 454926; 465497; 454865; 471032; 456855; 464469; 459388;
456370; 457453; 461267; 462325; 470057; 464116; 473071; 463471; 469589; 469101; 451497; 459100; 458014; 452162; 471149; 458288; 455710; 460878; 466507;
455600; 471961; 460857; 456632; 464804; 455661; 472417; 455538; 454329; 462344; 451506; 454028; 458845; 457347; 459792; 462454; 462866; 453112; 450828;
457120; 458198; 452070; 457986; 452543; 450298; 454459; 453444; 459331; 467064; 461206; 453154; 469007; 470332; 462572; 466759; 457683; 455719; 466852;
455704; 461473; 465315; 460392; 467058; 473099; 452046; 462647; 468451; 457461; 461721; 453987; 455309; 454877; 451618; 452793; 467011; 456981; 465035;
451573; 456678; 466790; 468160; 463692; 469237; 458196; 469200; 461320; 452729; 467642; 465752; 457820; 451228; 459711; 454067; 472731; 466076; 455748;
450160; 451709; 472169; 461425; 452900; 473746; 462103; 473522; 467036; 462652; 461402; 460911; 470744; 450943; 453344; 468972; 463546; 458088; 466669;
450977; 468943; 457954; 469031; 462651; 461810; 465511; 450109; 454668; 469129; 453122; 450344; 454761; 468754; 466765; 461324; 453179; 458191; 470965;
463235; 472218; 462984; 467031; 451963; 459210; 452850; 469743; 466791; 471545; 469019; 464853; 456556; 468223; 449613; 452403; 454542
human subset 1 (dm)

| | | |
|---|---|---|
| 408 | ARRB1 | arrestin, beta 1 |
| 6376 | CX3CL1 | chemokine (C—X3—C motif) ligand 1 |
| 2550 | GABBR1 | gamma-aminobutyric acid (GABA) B receptor, 1 |
| 4843 | NOS2A | nitric oxide synthase 2A (inducible, hepatocytes) |
| 9378 | NRXN1 | neurexin 1 |

human subset 2 (dm)
1213; 1231; 1234; 1445; 1855; 1856; 2268; 2798; 3579; 4792; 4905; 4985; 5144; 5744; 5745; 5879; 5900; 6553; 7201; 9266; 9368; 9568; 11248; 11249; 22809; 22871; 22895; 23026; 23037; 23499; 26037; 27111;
30010; 54413; 54843; 57555; 57586; 84958; 94120; 127833; 143425; 148281; 152789
human subset 3 (dm)
81; 102; 153; 154; 156; 163; 185; 332; 382; 407; 409; 468; 596; 613; 728; 857; 1213; 1231; 1234; 1237; 1238; 1316; 1445; 1524; 1544; 1571; 1855; 1856; 1956; 2118; 2150; 2268; 2353; 2357; 2554;
2566; 2776; 2798; 2911; 3265; 3329; 3458; 3480; 3552; 3553; 3557; 3565; 3569; 3579; 3586; 3600; 4193; 4288; 4301; 4524; 4790; 4792; 4842; 4844; 4845; 4846; 4905; 4985; 5144; 5590; 5594;
5595; 5599; 5600; 5734; 5739; 5743; 5744; 5745; 5879; 5900; 5970; 6361; 6367; 6541; 6553; 6714; 6772; 7020; 7073; 7076; 7124; 7157; 7201; 7422; 9266; 9368; 9369; 9379; 9568; 9832; 11009;
22809; 22871; 54413; 55922; 57586; 152789; 201288; 344697
chimpanzee subset 1 (dm)
454115; 455026
chimpanzee subset 2 (dm)
450128; 455236; 471226; 470644; 467426; 461860; 473393; 461008; 463253; 454163; 472842; 456181; 454865; 455105; 470999; 452655; 471579; 456778; 458032; 473660; 451723; 468441; 466499; 456673;
469641; 466425
chimpanzee subset 3 (dm)
456010; 453472; 466206; 471690; 451363; 460762; 452892; 454450; 468334; 468931; 463670; 450128; 470795; 460298; 450269; 460281; 453745; 450857; 455236; 454715; 461800; 453047; 456254;
462241; 462242; 471226; 466302; 459852; 449517; 453676; 470485; 450200; 459501; 449565; 463288; 470644; 469657; 450866; 463135; 461406; 467426; 467139; 461860; 454037; 450445; 450195;
456154; 473393; 461008; 463253; 466665; 467985; 467984; 452516; 454163; 458229; 470721; 450776; 472842; 456181; 454865; 462172; 457683; 470999; 473660; 466499; 471130
human subset 1 (k)

| | | |
|---|---|---|
| 51129 | ANGPTL4 | angiopoietin-like 4 |
| 6696 | SPP1 | secreted phosphoprotein 1 (osteopontin, bone sialoprotein I, early T-lymphocyte activation 1) |
| 10457 | GPNMB | glycoprotein (transmembrane) nmb |
| 1191 | CLU | clusterin |
| 7076 | TIMP1 | TIMP metallopeptidase inhibitor 1 |
| 3958 | LGALS3 | lectin, galactoside-binding, soluble, 3 (galectin 3) |
| 2244 | FGB | fibrinogen beta chain |
| 2243 | FGA | fibrinogen alpha chain |
| 4175 | MCM6 | MCM6 minichromosome maintenance deficient 6 (MIS5 homolog, S. pombe) (S. cerevisiae) |
| 6357 | CCL13 | chemokine (C-C motif) ligand 13 |
| 3827 | KNG1 | kininogen 1 |
| 960 | CD44 | CD44 molecule (Indian blood group) |
| 3627 | CXCL10 | chemokine (C—X—C motif) ligand 10 |
| 7345 | UCHL1 | ubiquitin carboxyl-terminal esterase L1 (ubiquitin thiolesterase) |
| 9966 | TNFSF15 | tumor necrosis factor (ligand) superfamily, member 15 |
| 1191 | CLU | clusterin |
| 54658 | UGT1A1 | UDP glucuronosyltransferase 1 family, polypeptide A1 |
| 1728 | NQO1 | NAD(P)H dehydrogenase, quinone 1 |
| 57016 | AKR1B10 | aldo-keto reductase family 1, member B10 (aldose reductase) |
| 2944 | GSTM1 | glutathione S-transferase M1 |
| 90637 | ZFAND2A | zinc finger, AN1-type domain 2A |
| 2940 | GSTA3 | glutathione S-transferase A3 |
| 2877 | GPX2 | glutathione peroxidase 2 (gastrointestinal) |
| 467 | ATF3 | activating transcription factor 3 |
| 3315 | HSPB1 | heat shock 27 kDa protein 1 |
| 83982 | FAM14A | family with sequence similarity 14, member A |
| 51768 | TM7SF3 | transmembrane 7 superfamily member 3 |
| 3158 | HMGCS2 | 3-hydroxy-3-methylglutaryl-Coenzyme A synthase 2 (mitochondrial) |
| 6713 | SQLE | squalene epoxidase |
| 302 | ANXA2 | annexin A2 |
| 6281 | S100A10 | S100 calcium binding protein A10 (annexin II ligand, calpactin I, light polypeptide (p11)) |
| 6277 | S100A6 | S100 calcium binding protein A6 (calcyclin) |
| 10568 | SLC34A2 | solute carrier family 34 (sodium phosphate), member 2 |
| 2980 | GUCA2A | guanylate cyclase activator 2A (guanylin) |
| 1559 | CYP2C9 | cytochrome P450, family 2, subfamily C, polypeptide 9 |
| 9390 | SLC22A13 | solute carrier family 22 (organic cation transporter), member 13 |
| 3176 | HNMT | histamine N-methyltransferase |
| 4942 | OAT | ornithine aminotransferase (gyrate atrophy) |
| 4953 | ODC1 | ornithine decarboxylase 1 |
| 1036 | CDO1 | cysteine dioxygenase, type I |
| 9104 | RGN | regucalcin (senescence marker protein-30) |
| 2974 | GUCY1B2 | guanylate cyclase 1, soluble, beta 2 |
| 2638 | GC | group-specific component (vitamin D binding protein) |
| 9833 | MELK | maternal embryonic leucine zipper kinase |
| 1733 | DIO1 | deiodinase, iodothyronine, type I |
| 29948 | OKL38 | pregnancy-induced growth inhibitor |
| 1733 | DIO1 | deiodinase, iodothyronine, type I |
| 29948 | OKL38 | pregnancy-induced growth inhibitor |
| 6622 | SNCA | synuclein, alpha (non A4 component of amyloid precursor) |
| 64222 | TOR3A | torsin family 3, member A |
| 25891 | DKFZP586H2123 | regeneration associated muscle protease |

human subset 2 (k)
350; 634; 972; 1277; 1278; 1445; 1462; 1508; 1649; 1758; 1839; 1956; 2035; 2066; 2244; 2247; 2266; 2534; 2547; 2885; 2946; 2984; 3273; 3303; 3486; 3661; 3674; 3690;
3725; 3727; 3848; 3932; 3952; 3953; 3959; 3982; 4018; 4036; 4088; 4176; 4217; 4267; 4316; 4318; 4771; 4790; 4811; 5104; 5111; 5245; 5327; 5329; 5340; 5345; 5444;
5447; 5552; 5621; 5970; 6275; 6277; 6281; 6285; 6401; 6696; 6714; 7046; 7048; 7054; 7057; 7074; 7157; 7345; 7373; 7410; 7430; 7436; 7448; 7529; 7531; 7804; 7846;
7916; 8029; 8174; 8291; 8550; 8605; 8878; 9138; 9261; 9479; 9627; 10048; 10248; 10434; 10987; 11319; 22918; 23059; 23136; 26353; 27101; 29124; 50626; 50628;
51365; 51684; 54205; 54675; 55388; 55503; 64386; 81620; 84570; 90441; 339403
human subset 3 (k)
9; 10; 25; 26; 43; 58; 59; 60; 70; 71; 102; 125; 126; 142; 196; 207; 231; 238; 301; 302; 303; 304; 305; 308; 309; 310; 311; 350; 351; 387; 440; 466; 468; 472; 498; 506; 571;
572; 581; 595; 598; 613; 624; 632; 633; 634; 649; 650; 654; 699; 718; 728; 730; 732; 735; 801; 811; 823; 824; 836; 842; 860; 861; 871; 920; 940; 958; 960; 963; 967; 968;
972; 994; 999; 1003; 1026; 1027; 1029; 1051; 1129; 1178; 1231; 1232; 1234; 1277; 1278; 1282; 1371; 1386; 1401; 1409; 1410; 1415; 1420; 1432; 1435; 1445; 1453; 1457;
1460; 1462; 1466; 1504; 1508; 1511; 1513; 1514; 1520; 1543; 1544; 1555; 1557; 1558; 1562; 1565; 1571; 1573; 1576; 1577; 1628; 1634; 1649; 1728; 1734; 1735; 1758;
1803; 1806; 1839; 1861; 1950; 1956; 1958; 1981; 1991; 1994; 2002; 2006; 2017; 2033; 2035; 2052; 2053; 2064; 2066; 2068; 2100; 2119; 2147; 2153; 2160; 2161; 2162;
2192; 2212; 2243; 2244; 2247; 2266; 2268; 2335; 2348; 2512; 2534; 2539; 2547; 2551; 2597; 2670; 2729; 2811; 2814; 2815; 2833; 2869; 2870; 2878; 2879; 2885; 2908;
2934; 2938; 2939; 2941; 2942; 2944; 2946; 2947; 2948; 2949; 2950; 2952; 2979; 2981; 2984; 3064; 3067; 3075; 3082; 3131; 3161; 3169; 3170; 3171; 3172; 3178; 3184;
3263; 3273; 3303; 3304; 3309; 3316; 3351; 3375; 3381; 3383; 3384; 3397; 3458; 3480; 3486; 3488; 3490; 3565; 3569; 3576; 3586; 3587; 3596; 3606; 3623; 3661; 3673;
3674; 3676; 3678; 3680; 3683; 3685; 3688; 3689; 3690; 3693; 3713; 3725; 3727; 3777; 3816; 3818; 3848; 3859; 3897; 3920; 3932; 3952; 3953; 3956; 3957; 3959; 3960;
3965; 3982; 4018; 4023; 4035; 4036; 4058; 4067; 4085; 4088; 4131; 4137; 4171; 4172; 4173; 4174; 4176; 4193; 4214; 4217; 4233; 4255; 4283; 4287; 4288; 4297; 4312;
4313; 4314; 4316; 4317; 4318; 4319; 4320; 4323; 4478; 4502; 4507; 4514; 4524; 4582; 4583; 4666; 4683; 4771; 4774; 4780; 4790; 4811; 4828; 4830; 4835; 4851; 4929;
4943; 4946; 4947; 4982; 5045; 5054; 5071; 5104; 5111; 5243; 5245; 5320; 5327; 5328; 5329; 5335; 5337; 5340; 5345; 5444; 5447; 5457; 5465; 5467; 5552; 5580; 5581;
5585; 5587; 5594; 5599; 5608; 5653; 5663; 5732; 5734; 5743; 5747; 5879; 5915; 5962; 5970; 5982; 5983; 5984; 5985; 6036; 6198; 6271; 6275; 6277; 6278; 6281; 6282;
6285; 6286; 6346; 6347; 6348; 6351; 6352; 6354; 6355; 6356; 6361; 6366; 6367; 6373; 6375; 6376; 6387; 6401; 6402; 6403; 6431; 6531; 6569; 6620; 6622; 6623; 6632;
6647; 6667; 6678; 6693; 6696; 6711; 6714; 6767; 6772; 6774; 6779; 6817; 6850; 6929; 7018; 7037; 7040; 7046; 7048; 7052; 7054; 7057; 7074; 7077; 7078; 7079; 7080;
7099; 7114; 7124; 7133; 7157; 7172; 7177; 7276; 7301; 7302; 7345; 7350; 7356; 7361; 7364; 7371; 7373; 7410; 7412; 7421; 7422; 7424; 7430; 7436; 7448; 7449; 7450;
7515; 7517; 7528; 7529; 7531; 7804; 7846; 7852; 8029; 8174; 8569; 8605; 8718; 8771; 8856; 8878; 8935; 8994; 9098; 9138; 9261; 9343; 9365; 9389; 9479; 9518; 9545;
9603; 9627; 9940; 9970; 9971; 10048; 10248; 10250; 10417; 10434; 10562; 10599; 10855; 10910; 10987; 11076; 11102; 11213; 11315; 11319; 11340; 22895; 22918;
22933; 22974; 23059; 23136; 23352; 23426; 26353; 27018; 29124; 50626; 51176; 51365; 54205; 54575; 54576; 54577; 54578; 54600; 54657; 54659; 54675; 55503; 56302;
56616; 56848; 64386; 64407; 64805; 79001; 80309; 81620; 84515; 84570; 85480; 90441; 91419; 94025; 120892; 142680; 339403; 375790; 440414; 654443
chimpanzee subset 1 (k)
461366; 464075; 461558; 461559; 460916; 461242; 461185; 473031; 464075; 460032; 468012; 463867; 463216; 462770; 467483; 463488; 467617; 457169; 464383; 453487; 457306; 471424; 469302; 450626; 470792;
450803; 459027; 462010; 465598; 461270; 465095; 469331; 454274; 469331; 454274; 461375; 457551
chimpanzee subset 2 (k)
468459; 462191; 463542; 461766; 463993; 452017; 456689; 459919; 461558; 461560; 458868; 473375; 460914; 463396; 454730; 468295; 456893; 449638; 456919; 454930; 463116; 459713; 472460;
465979; 451509; 461406; 453132; 458081; 455205; 472748; 450103; 463547; 463481; 450503; 458076; 466665; 457320; 457306; 450191; 461366; 458229; 472992; 460243; 450955; 453319; 461185;
464967; 454409; 451875; 471965; 456067; 451148; 462444; 463608; 466759; 450892; 458137; 467890; 455309; 452295; 457537; 469081; 464466; 470894; 465522; 458087; 468072; 461736
chimpanzee subset 3 (k)
464023; 464802; 463895; 466148; 450133; 453307; 454959; 453472; 461396; 457785; 453202; 463744; 459126; 472953; 453487; 461466; 462198; 450553; 468459;
463556; 452429; 468334; 455397; 451999; 458500; 451291; 458156; 467547; 473829; 464042; 458090; 459484; 455644; 468931; 461902; 456887; 453100; 455756;
451317; 457765; 456457; 472017; 466899; 450124; 470617; 451969; 468440; 462191; 458064; 465021; 469969; 463758; 470810; 450128; 463542; 460544; 457428;
458589; 462643; 457127; 454989; 461766; 452089; 463993; 467413; 457278; 457277; 453745; 456052; 450909; 470228; 450857; 456896; 463579; 456184; 452120;
452017; 468012; 467521; 459689; 457047; 462097; 460888; 468637; 455668; 473591; 458862; 456689; 457777; 464074; 454636; 459919; 456124; 460906; 451158;
457500; 462304; 462413; 458908; 450194; 461559; 461558; 461560; 451401; 458868; 451783; 454741; 462776; 468166; 466217; 471760; 471703; 471681; 462769;
462771; 457128; 457094; 451717; 472007; 469301; 473375; 453425; 457602; 463503; 455140; 456143; 458269; 461216; 450992; 460914; 464733; 451544; 462833;
471249; 450196; 469912; 449517; 453676; 463396; 459933; 449565; 463288; 471205; 469657; 451580; 454730; 470596; 451958; 470789; 459807; 450396; 458607;
468295; 456893; 470334; 468971; 471375; 468241; 449638; 456919; 470204; 454930; 456011; 454519; 463116; 464031; 452006; 459713; 453350; 472771; 461661;
450137; 460668; 462763; 472757; 458790; 472460; 471543; 463671; 471200; 453114; 450866; 451587; 451510; 454094; 451511; 451509; 458701; 471221; 807869;
452434; 464278; 459769; 461406; 455134; 462395; 459669; 464351; 453652; 472467; 453132; 458081; 455205; 456587; 472748; 450103; 470996; 463547; 463481;
450503; 459203; 452834; 450445; 468310; 467503; 467459; 450195; 464422; 463253; 466776; 466665; 463467; 452533; 460918; 473325; 457325; 457320; 469501;
457306; 457994; 455004; 454579; 468209; 468208; 467985; 467984; 469572; 454115; 450191; 469946; 462303; 461375; 450574; 451939; 471709; 467939; 461366;
459234; 458229; 470721; 454013; 468650; 460710; 460956; 456060; 472992; 460243; 450955; 453319; 454928; 458784; 460176; 452867; 457968; 453352; 461185;
461504; 466634; 471218; 457479; 451858; 471358; 464967; 456094; 453191; 454409; 451875; 450130; 469189; 469784; 458417; 463306; 455259; 456067; 451148;
468770; 468721; 463298; 470790; 458014; 452162; 462444; 463608; 456632; 467367; 465331; 461206; 452744; 466759; 461690; 470831; 467058; 465864; 450892;
467263; 458137; 456013; 458157; 467890; 455309; 452295; 465780; 469081; 464466; 461425; 470894; 465522; 458087; 468943; 457592; 460119; 468072; 458449;
461736; 452031; 465392; 455173
human subset 1 (u)

| | | |
|---|---|---|
| 5757 | PTMA | prothymosin, alpha (gene sequence 28) |
| 3397 | ID1 | inhibitor of DNA binding 1, dominant negative helix-loop-helix protein |
| 1280 | COL2A1 | collagen, type II, alpha 1 (primary osteoarthritis, spondyloepiphyseal dysplasia, congenital) |
| 635 | BHMT | betaine-homocysteine methyltransferase |
| 1565 | CYP2D6 | cytochrome P450, family 2, subfamily D, polypeptide 6 |
| 1652 | DDT | D-dopachrome tautomerase |
| 5444 | PON1 | paraoxonase 1 |
| 30 | ACAA1 | acetyl-Coenzyme A acyltransferase 1 (peroxisomal 3-oxoacyl-Coenzyme A thiolase) |
| 30 | ACAA1 | acetyl-Coenzyme A acyltransferase 1 (peroxisomal 3-oxoacyl-Coenzyme A thiolase) |
| 341 | APOC1 | apolipoprotein C-I |
| 6582 | SLC22A2 | solute carrier family 22 (organic cation transporter), member 2 |
| 9356 | SLC22A6 | solute carrier family 22 (organic anion transporter), member 6 |
| 9104 | RGN | regucalcin (senescence marker protein-30) |
| 443 | ASPA | aspartoacylase (Canavan disease) |
| 4311 | MME | membrane metallo-endopeptidase (neutral endopeptidase, enkephalinase, CALLA, CD10) |
| 1145 | CHRNE | cholinergic receptor, nicotinic, epsilon |
| 596 | BCL2 | B-cell CLL/lymphoma 2 |
| 836 | CASP3 | caspase 3, apoptosis-related cysteine peptidase |
| 1270 | CNTF | ciliary neurotrophic factor |
| 2152 | F3 | coagulation factor III (thromboplastin, tissue factor) |
| 302 | ANXA2 | annexin A2 |
| 1520 | CTSS | cathepsin S |
| 1647 | GADD45A | growth arrest and DNA-damage-inducible, alpha |
| 126 | ADH1C | alcohol dehydrogenase 1C (class I), gamma polypeptide |
| 435 | ASL | argininosuccinate lyase |
| 2710 | GK | glycerol kinase |
| 51733 | UPB1 | ureidopropionase, beta |
| 221357 | GSTA5 | glutathione S-transferase A5 |
| 2950 | GSTP1 | glutathione S-transferase pi |
| 221357 | GSTA5 | glutathione S-transferase A5 |
| 5351 | PLOD1 | procollagen-lysine 1,2-oxoglutarate 5-dioxygenase 1 |
| 3417 | IDH1 | isocitrate dehydrogenase 1 (NADP+), soluble |
| 847 | CAT | catalase |
| 2937 | GSS | glutathione synthetase |
| 3091 | HIF1A | hypoxia-inducible factor 1, alpha subunit (basic helix-loop-helix transcription factor) |
| 2 | A2M | alpha-2-macroglobulin |
| 2243 | FGA | fibrinogen alpha chain |
| 1316 | KLF6 | Kruppel-like factor 6 |
| 308 | ANXA5 | annexin A5 |
| 1191 | CLU | clusterin |
| 8655 | DYNLL1 | dynein, light chain, LC8-type 1 |
| 1649 | DDIT3 | DNA-damage-inducible transcript 3 |
| 203054 | ADCK5 | aarF domain containing kinase 5 |
| 1647 | GADD45A | growth arrest and DNA-damage-inducible, alpha |
| 4673 | NAP1L1 | nucleosome assembly protein 1-like 1 |
| 2168 | FABP1 | fatty acid binding protein 1, liver |

human subset 2 (u)
259; 348; 350; 354; 567; 598; 634; 637; 638; 640; 662; 663; 664; 665; 836; 841; 842; 985; 1017; 1271; 1280; 1291; 1298; 1299; 1300; 1311; 1499; 1508; 1630; 1634; 1639;
1676; 1822; 1958; 1975; 1990; 2155; 2159; 2244; 2252; 2266; 2316; 2335; 2353; 2547; 2885; 3059; 3064; 3164; 3265; 3273; 3303; 3309; 3320; 3329; 3336; 3553; 3570;
3572; 3586; 3674; 3690; 3693; 3725; 3727; 3791; 3827; 3845; 3931; 3952; 3953; 3977; 4001; 4035; 4036; 4099; 4146; 4193; 4216; 4267; 4287; 4616; 4719; 4756; 4780;
4803; 4811; 4869; 4893; 5047; 5104; 5111; 5155; 5187; 5230; 5245; 5291; 5295; 5300; 5310; 5327; 5335; 5340; 5345; 5366; 5444; 5465; 5468; 5499; 5515; 5525; 5530;
5549; 5594; 5595; 5599; 5600; 5621; 5663; 5688; 5702; 5781; 5883; 5894; 5901; 5925; 6009; 6189; 6237; 6256; 6275; 6277; 6281; 6317; 6318; 6464; 6606; 6647; 6667;
6678; 6714; 6721; 6731; 6774; 7009; 7040; 7045; 7046; 7048; 7052; 7057; 7157; 7159; 7185; 7187; 7422; 7428; 7433; 7436; 7536; 7804; 7818; 7916; 8290; 8291; 8301;
8339; 8349; 8359; 8370; 8505; 8573; 8615; 8648; 8678; 8717; 8726; 8728; 8739; 8797; 8837; 9026; 9088; 9112; 9191; 9229; 9314; 9445; 9510; 9530; 9531; 9611; 9748;
9774; 9804; 9924; 10017; 10018; 10134; 10243; 10383; 10434; 10846; 10912; 10956; 10987; 11142; 11331; 23059; 23139; 23636; 23770; 26052; 27113; 29124; 30818;
51177; 51283; 51365; 54205; 54583; 54938; 55662; 56288; 57142; 57448; 57619; 57761; 63970; 64112; 64344; 64386; 64689; 79370; 79444; 83985; 84171; 84817; 84951;
90427; 90480; 112399; 121504; 126961; 149428; 391104
human subset 3 (u)
25; 27; 30; 43; 51; 60; 71; 118; 124; 125; 131; 133; 140; 142; 174; 176; 183; 185; 190; 207; 213; 217; 238; 244; 259; 285; 290; 291; 301; 302; 303; 304; 305; 306; 308; 309;
317; 324; 328; 329; 330; 331; 332; 336; 342; 348; 350; 351; 354; 355; 356; 358; 367; 383; 387; 392; 397; 405; 440; 466; 467; 468; 472; 488; 493; 498; 506; 567; 572; 573;
578; 580; 581; 590; 595; 596; 597; 598; 599; 604; 613; 632; 633; 634; 637; 638; 640; 641; 650; 652; 654; 660; 662; 663; 664; 665; 672; 712; 730; 732; 735; 768; 780; 823;
824; 831; 834; 835; 836; 837; 838; 839; 840; 841; 842; 843; 847; 891; 921; 924; 949; 959; 960; 969; 983; 985; 990; 998; 999; 1000; 1001; 1003; 1017; 1026; 1027; 1029;
1050; 1051; 1053; 1054; 1071; 1075; 1101; 1128; 1131; 1134; 1137; 1140; 1144; 1146; 1191; 1234; 1271; 1277; 1278; 1280; 1281; 1282; 1284; 1285; 1286; 1290; 1291;
1292; 1297; 1298; 1299; 1300; 1301; 1311; 1356; 1386; 1387; 1401; 1427; 1432; 1435; 1437; 1457; 1471; 1473; 1499; 1508; 1509; 1510; 1511; 1512; 1513; 1514; 1537;
1543; 1544; 1557; 1558; 1559; 1564; 1568; 1571; 1574; 1576; 1577; 1612; 1630; 1634; 1636; 1638; 1639; 1647; 1648; 1649; 1662; 1676; 1719; 1728; 1742; 1807; 1813;
1814; 1822; 1843; 1869; 1889; 1906; 1950; 1956; 1958; 1965; 1975; 1990; 1994; 2002; 2017; 2022; 2033; 2034; 2035; 2052; 2056; 2057; 2064; 2067; 2099; 2100; 2147;
2149; 2150; 2155; 2158; 2159; 2162; 2167; 2169; 2172; 2173; 2185; 2194; 2237; 2244; 2247; 2252; 2266; 2272; 2316; 2335; 2353; 2355; 2475; 2520; 2521; 2534; 2547;
2597; 2620; 2623; 2670; 2678; 2690; 2711; 2712; 2713; 2714; 2716; 2729; 2735; 2784; 2803; 2821; 2876; 2885; 2932; 2934; 2936; 2938; 2939; 2944; 2946; 2950; 2952;
2953; 3002; 3059; 3065; 3066; 3067; 3082; 3084; 3099; 3108; 3131; 3162; 3164; 3172; 3178; 3184; 3190; 3265; 3273; 3303; 3309; 3312; 3316; 3329; 3336; 3398; 3399;
3458; 3479; 3480; 3481; 3485; 3486; 3492; 3553; 3557; 3565; 3569; 3570; 3572; 3576; 3586; 3600; 3603; 3623; 3672; 3674; 3690; 3693; 3716; 3717; 3725; 3727; 3791;
3827; 3845; 3855; 3859; 3875; 3931; 3952; 3953; 3976; 3977; 4023; 4035; 4036; 4055; 4058; 4067; 4086; 4087; 4088; 4090; 4092; 4099; 4137; 4146; 4170; 4193; 4205;
4209; 4214; 4215; 4216; 4217; 4254; 4255; 4282; 4286; 4288; 4303; 4311; 4312; 4313; 4318; 4322; 4353; 4363; 4436; 4502; 4524; 4609; 4616; 4634; 4637; 4644; 4654;
4656; 4666; 4691; 4719; 4756; 4780; 4790; 4791; 4792; 4800; 4801; 4802; 4803; 4811; 4828; 4842; 4843; 4869; 4880; 4893; 4915; 4921; 4928; 4961; 4988; 5045; 5047;
5052; 5054; 5062; 5076; 5104; 5111; 5155; 5159; 5187; 5209; 5230; 5241; 5243; 5245; 5268; 5272; 5291; 5295; 5300; 5310; 5327; 5329; 5335; 5340; 5345; 5352; 5359;
5366; 5371; 5443; 5444; 5445; 5446; 5447; 5450; 5451; 5452; 5465; 5468; 5499; 5515; 5525; 5530; 5569; 5570; 5580; 5586; 5588; 5590; 5591; 5594; 5595; 5599; 5600;
5603; 5604; 5610; 5617; 5621; 5663; 5688; 5702; 5725; 5728; 5743; 5747; 5757; 5759; 5760; 5763; 5777; 5781; 5819; 5829; 5830; 5858; 5867; 5883; 5888; 5894; 5915;
5916; 5925; 5970; 5981; 5982; 5983; 5984; 5985; 6095; 6189; 6195; 6198; 6237; 6240; 6252; 6275; 6277; 6281; 6317; 6318; 6347; 6416; 6418; 6464; 6477; 6478; 6489;
6502; 6513; 6515; 6521; 6530; 6532; 6571; 6580; 6581; 6590; 6606; 6622; 6647; 6648; 6662; 6667; 6670; 6688; 6714; 6721; 6731; 6772; 6774; 6776; 6781; 6788; 6789;
6921; 6923; 6929; 7001; 7009; 7015; 7018; 7035; 7040; 7043; 7045; 7046; 7048; 7052; 7057; 7114; 7124; 7132; 7150; 7157; 7158; 7159; 7177; 7185; 7186; 7187; 7278;
7298; 7301; 7302; 7350; 7416; 7422; 7428; 7431; 7433; 7436; 7450; 7490; 7531; 7534; 7536; 7782; 7804; 7852; 7917; 7941; 8125; 8260; 8301; 8339; 8370; 8505; 8573;
8615; 8647; 8648; 8655; 8678; 8728; 8739; 8740; 8743; 8772; 8797; 8837; 8841; 8850; 8932; 8985; 9088; 9093; 9135; 9191; 9229; 9314; 9365; 9376; 9445; 9451; 9459;
9507; 9510; 9531; 9545; 9611; 9748; 9774; 9804; 9915; 9941; 9942; 9943; 9982; 10017; 10018; 10059; 10134; 10155; 10168; 10197; 10243; 10376; 10381; 10395; 10417;
10434; 10452; 10482; 10541; 10746; 10763; 10846; 10864; 10912; 10956; 10987; 11096; 11142; 11168; 11331; 22926; 22974; 23059; 23368; 23621; 23645; 23770; 23786;
26003; 26355; 26986; 27113; 28988; 29124; 30818; 50488; 51024; 51126; 51143; 51177; 51283; 51365; 51564; 51738; 51765; 54205; 54583; 54938; 55503; 55662; 55867;
56302; 56616; 56850; 57142; 57761; 59272; 59348; 63970; 64344; 64386; 64689; 64805; 79370; 79444; 80196; 83985; 84171; 84817; 84951; 90427; 90480; 91419;
112398; 112399; 121504; 126961; 140735; 149428; 162989; 163732; 171017; 192343; 285605; 326626; 338025; 386607; 391104; 406948; 406950; 440085; 654443
chimpanzee subset 1 (u)
460522; 469912; 470228; 463547; 460268; 460268; 463113; 451273; 465598; 454430; 460792; 457038; 453487; 457277; 456929; 463444; 451717; 457956; 451116; 452958; 461559; 450269; 461466; 464075; 452017;
472898; 456929; 452083
chimpanzee subset 2 (u)
468459; 450170; 458156; 458635; 470232; 463991; 450831; 464069; 460110; 456457; 473218; 456791; 469068; 463993; 455426; 452120; 459332; 457928; 462097;
451925; 452679; 461558; 461560; 453047; 458868; 466302; 460914; 464733; 459852; 452179; 450200; 469506; 461866; 469657; 454730; 468295; 456893; 461315;
460916; 473387; 449638; 456919; 452006; 459713; 469075; 463119; 465979; 453114; 459896; 453729; 459769; 462261; 464845; 453132; 458081; 458844; 455098;
473678; 455205; 471079; 461836; 454340; 472748; 463547; 460178; 471633; 469649; 454037; 450445; 458076; 467503; 458383; 451172; 467135; 466893; 460182;
452414; 452715; 463880; 468955; 464834; 457320; 457306; 455459; 457351; 451939; 471709; 458229; 451883; 456060; 462081; 472992; 460243; 453319; 464702;
453178; 460291; 464967; 466656; 458006; 471965; 451725; 471883; 469477; 462507; 461243; 454700; 454154; 466901; 459872; 467880; 453217; 457448; 464640;
452714; 470057; 464116; 468370; 450716; 467685; 459487; 452976; 464585; 452022; 466759; 451805; 467890; 456568; 455862; 457514; 469081; 459401; 457268;
453937; 470894; 465522; 466183; 459138; 461235; 469852; 453122; 460278; 465293; 466175; 455246; 453323; 452850; 457281
chimpanzee subset 3 (u)
464802; 457552; 460268; 454895; 450133; 454959; 461396; 471257; 451021; 457785; 461258; 453629; 450104; 460762; 450140; 453202; 461260; 452252; 459126;
467758; 472953; 453487; 461466; 462198; 452153; 461999; 465200; 451508; 451507; 473765; 458013; 468459; 466149; 469584; 463330; 465680; 462999; 451156;
457279; 463556; 452429; 468334; 455397; 451999; 450170; 451291; 465041; 462616; 470638; 460830; 468156; 452796; 473829; 458635; 470232; 463991; 453650;
458090; 450831; 464069; 449497; 461902; 456887; 473223; 451317; 457765; 462348; 451519; 463804; 451518; 450745; 460110; 456457; 459873; 451116; 461835;
452359; 473802; 465280; 450481; 468234; 456609; 468509; 465021; 455931; 469969; 468818; 467982; 451469; 469725; 470583; 469999; 454461; 470680; 464075;
450128; 473218; 463542; 459815; 459988; 459986; 462810; 456791; 469068; 467891; 457428; 460909; 462643; 457127; 471627; 463993; 457677; 467413; 453574;
457278; 472893; 453745; 450909; 450626; 450857; 463579; 455426; 452120; 454782; 452622; 459332; 456929; 452017; 457928; 461773; 468012; 468444; 464330;
451553; 470885; 471742; 458184; 456594; 462437; 462097; 452980; 451925; 455668; 473591; 458862; 459204; 456689; 457777; 463609; 455728; 454636; 463079;
451158; 471510; 461800; 465887; 452679; 462413; 464255; 462233; 472116; 464073; 454986; 451242; 461558; 461560; 470835; 453047; 459115; 468262; 458868;
451783; 451081; 465619; 454741; 470168; 471560; 471192; 462776; 460264; 455941; 470895; 462769; 451717; 458962; 464259; 462950; 453425; 463503; 449592;
455140; 470195; 458269; 461216; 464554; 466302; 460914; 464733; 451629; 451544; 459852; 452179; 459009; 456618; 449517; 453676; 466318; 459932; 463396;
450200; 459501; 449565; 463288; 469506; 461866; 471205; 469657; 449644; 454730; 468295; 456911; 456893; 461315; 460916; 473387; 466983; 468241; 449638;
456919; 464031; 452006; 459713; 451778; 453350; 472771; 450142; 455407; 455411; 469075; 457274; 471543; 468286; 463119; 452114; 450866; 473659; 460792;
451510; 454094; 451513; 459215; 471221; 460327; 451057; 452434; 459896; 453729; 459769; 461406; 467426; 452191; 456799; 467139; 455026; 462261; 470677;
457467; 450971; 472165; 453652; 464845; 456537; 472467; 460965; 453132; 458081; 458844; 462188; 455098; 450287; 473678; 451502; 455205; 468566; 471079;
461836; 454340; 472748; 450103; 460754; 460757; 453736; 459074; 463547; 463549; 463548; 463481; 451537; 456070; 460178; 471633; 472115; 456998; 450288;
464165; 454037; 450445; 462644; 453526; 459155; 462468; 458076; 467503; 458383; 451172; 468635; 466142; 464422; 460522; 467135; 451812; 465371; 466893;
453339; 460182; 451935; 452715; 466665; 461174; 463467; 452533; 460918; 468955; 452945; 457320; 457306; 455459; 455065; 457351; 465153; 460781; 465341;
471570; 451818; 454096; 466213; 472177; 461375; 449634; 450101; 451939; 459750; 458229; 470721; 454681; 472719; 472829; 458275; 464236; 453843; 468650;
468740; 451883; 471443; 460710; 470603; 456060; 453054; 462081; 472992; 460243; 453319; 466934; 472883; 467668; 464702; 464876; 453178; 453352; 461504;
471632; 460291; 464967; 454409; 472830; 466656; 453403; 450130; 462560; 462744; 465931; 451725; 471883; 461243; 454700; 468687; 460852; 451389; 459872;
460219; 463618; 467880; 453879; 455255; 457448; 464640; 451274; 452714; 459388; 473803; 457453; 470057; 468721; 468370; 450716; 453583; 470791; 471149;
467685; 459487; 465266; 456353; 460307; 452976; 454329; 464001; 451267; 464605; 462710; 464585; 452022; 466759; 451805; 457461; 458157; 467890; 453193;
451573; 456191; 455862; 463925; 469081; 459401; 454455; 457268; 453937; 470894; 466968; 470744; 465522; 466183; 469852; 465511; 460278; 465293; 467145;
466175; 455246; 453323; 452031; 452850; 457281; 471610; 465443
human subset 1 (vl)
human subset 2 (vl)
1649; 2074; 2534; 2885; 2965; 3554; 3556; 3570; 3572; 3577; 3579; 3635; 3636; 3725; 3727; 4067; 4088; 4790; 4913; 5111; 5295; 5296; 5328; 5340; 5430; 5657; 5744; 5921; 5925; 5970; 6351; 6382; 6885; 7157;
7525; 7538; 7850; 7920; 8290; 8563; 8651; 8841; 9021; 9612; 10449; 55054; 83734
human subset 3 (vl)
2; 50; 51; 102; 136; 142; 154; 177; 183; 196; 207; 239; 240; 246; 285; 329; 332; 351; 355; 367; 387; 440; 466; 468; 472; 595; 596; 597; 598; 624; 627; 632; 660; 708; 711;
765; 768; 820; 834; 837; 838; 841; 857; 861; 862; 867; 890; 904; 929; 940; 943; 944; 952; 958; 959; 960; 968; 972; 983; 998; 1025; 1026; 1027; 1051; 1052; 1113; 1114;
1118; 1147; 1161; 1230; 1280; 1294; 1312; 1386; 1401; 1432; 1435; 1437; 1439; 1440; 1470; 1499; 1508; 1509; 1514; 1557; 1559; 1571; 1588; 1601; 1634; 1636; 1649;
1673; 1803; 1869; 1890; 1903; 1906; 1950; 1956; 1958; 1977; 1991; 1994; 2000; 2026; 2033; 2056; 2064; 2067; 2068; 2071; 2074; 2118; 2147; 2149; 2150; 2152; 2160;
2208; 2237; 2244; 2247; 2261; 2266; 2309; 2323; 2475; 2492; 2495; 2520; 2521; 2532; 2534; 2547; 2623; 2771; 2778; 2885; 2919; 2921; 2932; 2950; 2952; 2965; 3002;
3055; 3077; 3082; 3091; 3156; 3162; 3169; 3170; 3171; 3184; 3205; 3248; 3265; 3274; 3297; 3320; 3383; 3397; 3439; 3456; 3458; 3479; 3485; 3500; 3514; 3551; 3552;
3553; 3554; 3556; 3557; 3558; 3559; 3562; 3565; 3569; 3570; 3572; 3574; 3575; 3576; 3577; 3579; 3581; 3586; 3587; 3589; 3592; 3593; 3596; 3603; 3605; 3606; 3627;
3635; 3636; 3643; 3658; 3725; 3727; 3778; 3815; 3827; 3909; 3931; 3932; 3952; 3976; 4035; 4049; 4050; 4052; 4055; 4067; 4072; 4088; 4089; 4091; 4092; 4153; 4170;
4193; 4233; 4254; 4282; 4312; 4313; 4314; 4316; 4317; 4318; 4321; 4322; 4323; 4330; 4502; 4583; 4586; 4590; 4615; 4683; 4772; 4773; 4778; 4780; 4790; 4793; 4830;
4843; 4846; 4881; 4893; 4913; 4924; 4982; 5008; 5027; 5054; 5058; 5074; 5111; 5196; 5243; 5271; 5273; 5292; 5295; 5296; 5321; 5327; 5328; 5329; 5337; 5338; 5340;
5465; 5468; 5473; 5564; 5578; 5580; 5581; 5583; 5590; 5592; 5594; 5595; 5599; 5600; 5601; 5604; 5624; 5657; 5663; 5700; 5730; 5731; 5732; 5733; 5734; 5740; 5742;
5743; 5744; 5745; 5771; 5788; 5791; 5803; 5804; 5879; 5887; 5915; 5921; 5925; 5970; 5971; 5979; 6019; 6174; 6195; 6240; 6261; 6280; 6285; 6287; 6288; 6289; 6337;
6346; 6347; 6348; 6351; 6352; 6354; 6356; 6364; 6372; 6374; 6382; 6387; 6399; 6401; 6403; 6416; 6502; 6513; 6518; 6564; 6574; 6688; 6714; 6774; 6778; 6794; 6916;
6927; 7037; 7040; 7042; 7046; 7066; 7072; 7073; 7097; 7098; 7099; 7100; 7124; 7132; 7133; 7157; 7177; 7186; 7187; 7188; 7189; 7253; 7295; 7371; 7412; 7421; 7422;
7424; 7450; 7496; 7520; 7525; 7538; 7850; 7852; 7913; 7980; 8061; 8563; 8570; 8607; 8651; 8772; 8837; 8856; 8870; 9021; 9140; 9370; 9407; 9518; 9536; 9641; 9672;
9970; 10381; 10388; 10449; 10597; 10850; 10855; 11283; 11337; 22861; 22914; 22993; 23516; 23621; 25913; 27035; 27087; 27189; 29108; 29127; 29949; 30061; 50604;
51561; 54106; 54474; 54577; 55054; 59082; 59340; 64407; 64422; 80142; 81285; 81631; 114548; 147945; 148738; 192343
chimpanzee subset 1 (vl)
465034; 462905; 462187; 457913; 452649; 450200; 463288; 471205; 452807; 456862; 455460
chimpanzee subset 2 (vl)
452017; 451061; 459460; 460935; 469506; 461866; 470645; 470644; 451403; 456893; 472771; 461406; 458081; 461836; 455855; 473393; 461762; 452715; 466665; 455004; 462888; 455322; 470455; 462563; 458742;
467150; 455414
chimpanzee subset 3 (vl)
454895; 453472; 455009; 457785; 471690; 462584; 450104; 453202; 455242; 451508; 466149; 465680; 463556; 452429; 468334; 458156; 467547; 455252; 457915;
473223; 470816; 451519; 451518; 460110; 463670; 451690; 461467; 462118; 470617; 457960; 464686; 473802; 468440; 462191; 450481; 456609; 469969; 464163;
470034; 457641; 450673; 471821; 460347; 458655; 457428; 462643; 457127; 471627; 458975; 454641; 458144; 463993; 450626; 450857; 453433; 461905; 452120;
454782; 452017; 459689; 458184; 458941; 464580; 462437; 462097; 461391; 468637; 455668; 458862; 463609; 454636; 456124; 470526; 454715; 451158; 471510;
461800; 457038; 456434; 451242; 461558; 461067; 461560; 456206; 470371; 468262; 458868; 465619; 469986; 461255; 470895; 451717; 451061; 458163; 462489;
463503; 452958; 471516; 470195; 456143; 461216; 461613; 466302; 471750; 450196; 469912; 473197; 473183; 449517; 459932; 472749; 470485; 450200; 459460;
460935; 459501; 461472; 450884; 471626; 449565; 463288; 469506; 461866; 464245; 461922; 471205; 470645; 470644; 469657; 451580; 460816; 471723; 449644;
472029; 461242; 451403; 455649; 467731; 456893; 461316; 460916; 449638; 452006; 459143; 451778; 472771; 459214; 468537; 467711; 455411; 466126; 457274;
463671; 452114; 451510; 454094; 451511; 451509; 451512; 451513; 458730; 471221; 460269; 464278; 455485; 469972; 459769; 461406; 456014; 455134; 455026;
469093; 464351; 452318; 472467; 451439; 452094; 458081; 455461; 468570; 462656; 461836; 455855; 472748; 450103; 470996; 454454; 460178; 452297; 459203;
454037; 450445; 453526; 459593; 467503; 467459; 450195; 458323; 464713; 473393; 461008; 455504; 457607; 450865; 463253; 473012; 461762; 452715; 466665;
456119; 450415; 449633; 457998; 466459; 451776; 455004; 454579; 468208; 459991; 471204; 471203; 465995; 450191; 455065; 471570; 457916; 467307; 459498;
458229; 452005; 468641; 463774; 460956; 456060; 457742; 472992; 459303; 450776; 471325; 471374; 457762; 466934; 457968; 464876; 453178; 457711; 450474;
450779; 457479; 451858; 471358; 449614; 455322; 470455; 450130; 462460; 463540; 458742; 455640; 451389; 459872; 462547; 462011; 471032; 471225; 468770;
473071; 458014; 454329; 458373; 455414; 461206; 468451; 451573; 463692; 465752; 466859; 468067; 454067; 451887; 459821; 467036; 468501; 457592; 470880;
466344; 469743; 469019; 469830
human subset 1 (ae)
human subset 2 (ae)
656; 657; 658; 659; 1031; 1857; 1869; 1877; 2268; 3091; 3692; 3875; 4036; 4087; 4088; 4171; 4193; 4609; 4646; 5300; 5310; 5317; 5318; 5327; 5339; 5411; 5581; 5599; 5602; 5702; 6464; 6714; 6885; 7046; 7048; 7133;
7399; 7486; 7508; 7528; 7529; 7531; 7532; 7533; 7534; 8646; 8717; 9191; 9241; 9611; 9612; 10049; 10075; 11187; 11284; 23181; 23468; 25928; 29950; 51776; 54840; 55602; 55620; 55914; 57534; 57862; 79661;
79800; 84687; 85415; 168667
human subset 3 (ae)
142; 207; 239; 242; 301; 328; 375; 388; 467; 472; 545; 581; 595; 596; 633; 648; 649; 650; 651; 653; 654; 655; 656; 657; 658; 659; 667; 672; 705; 836; 839; 840; 864; 867;
891; 894; 896; 898; 928; 930; 977; 983; 999; 1019; 1021; 1026; 1027; 1028; 1030; 1031; 1032; 1111; 1173; 1280; 1308; 1380; 1410; 1432; 1438; 1445; 1457; 1600; 1612;
1639; 1728; 1786; 1788; 1789; 1832; 1857; 1869; 1874; 1875; 1877; 1906; 1950; 1956; 1958; 1977; 1978; 2001; 2002; 2033; 2064; 2067; 2068; 2071; 2099; 2125; 2185;
2268; 2272; 2335; 2534; 2537; 2688; 2689; 2694; 2720; 2778; 2810; 2885; 2950; 3082; 3083; 3091; 3265; 3309; 3315; 3397; 3551; 3655; 3673; 3675; 3685; 3688; 3690;
3691; 3692; 3725; 3845; 3848; 3850; 3852; 3856; 3861; 3875; 3880; 3908; 3911; 3912; 3915; 3980; 4000; 4036; 4086; 4087; 4088; 4089; 4090; 4092; 4157; 4162; 4193;
4214; 4216; 4233; 4255; 4286; 4287; 4292; 4316; 4350; 4436; 4485; 4486; 4489; 4502; 4507; 4609; 4610; 4641; 4646; 4763; 4790; 4869; 4893; 4968; 5087; 5111; 5179;
5300; 5310; 5327; 5329; 5330; 5339; 5411; 5423; 5493; 5578; 5580; 5581; 5585; 5594; 5595; 5599; 5600; 5602; 5603; 5604; 5606; 5609; 5701; 5702; 5710; 5743; 5886;
5887; 5896; 5914; 5915; 5925; 5970; 6095; 6197; 6300; 6401; 6416; 6464; 6521; 6591; 6598; 6667; 6678; 6690; 6692; 6714; 6722; 6775; 6885; 6929; 7015; 7027; 7045;
7046; 7048; 7056; 7114; 7132; 7133; 7150; 7157; 7161; 7187; 7216; 7298; 7341; 7374; 7399; 7422; 7428; 7431; 7474; 7486; 7490; 7508; 7517; 7520; 7528; 7529; 7531;
7532; 7533; 7534; 8004; 8200; 8646; 8701; 8717; 8840; 8900; 9021; 9191; 9241; 9261; 9344; 9370; 9423; 9611; 9612; 9635; 9836; 10024; 10049; 10075; 10221; 10272;
10309; 10468; 10808; 11186; 11259; 11284; 23181; 23468; 23492; 23604; 23627; 23671; 25928; 25942; 29127; 29950; 29978; 30011; 51535; 51776; 54840; 55620; 55914;
56924; 57045; 57534; 57551; 57862; 79576; 84687; 85415; 89780; 114757; 153090; 162989; 168667; 204010; 286887
chimpanzee subset 1 (ae)
461905; 456094; 467980; 468310; 468770; 468863; 465021
chimpanzee subset 2 (ae)
450572; 460882; 458184; 454356; 452958; 458200; 459713; 455407; 460668; 454340; 465264; 472748; 452875; 459203; 450445; 451172; 457351; 458229; 462888; 472992; 460243; 457968; 457738; 472732; 460195;
454409; 458778; 472830; 460892; 454154; 457448; 468413; 468370; 467150; 463847; 465650; 467011; 463274; 456037; 459745; 461623; 471527; 467829; 459889; 465382
chimpanzee subset 3 (ae)
457785; 453202; 455242; 455103; 472953; 465200; 459054; 460745; 473829; 466023; 464042; 458090; 462787; 450572; 449497; 450745; 451690; 461835; 462689;
455914; 451775; 454020; 450481; 452025; 463534; 450717; 457690; 462643; 456888; 459332; 468012; 455696; 458174; 462420; 460882; 458184; 454158; 454356;
462437; 462097; 461391; 473591; 458862; 454636; 456124; 470526; 463079; 454896; 464073; 470835; 450178; 460254; 469986; 451717; 463503; 471126; 452958;
466302; 464733; 463488; 469912; 472749; 459742; 455111; 459807; 450396; 468295; 458200; 456893; 473387; 466998; 454664; 472122; 454581; 457385; 459713;
450142; 455407; 468537; 455411; 471543; 463119; 463671; 453114; 471095; 451509; 459215; 460377; 471221; 454566; 461406; 462261; 460152; 458081; 464186;
454340; 472748; 450103; 452875; 453893; 459203; 454037; 450445; 462644; 453526; 454515; 463632; 451172; 473012; 466489; 452715; 466665; 450110; 450191;
455065; 457351; 464169; 458702; 451939; 471709; 453342; 458229; 462709; 459834; 462888; 468650; 471443; 462081; 472992; 460243; 469898; 466934; 457968;
472883; 453178; 459882; 452217; 457738; 472732; 460195; 453191; 449614; 454409; 458778; 472830; 460892; 472305; 454154; 472862; 467260; 457448; 468413;
471032; 455083; 468370; 467150; 451878; 463847; 465650; 455531; 461879; 460392; 467011; 458843; 469871; 463274; 453759; 451887; 456037; 465527; 451837;
459745; 471527; 453326; 465382; 469698; 468337
human subset 1 (d)

| | | |
|---|---|---|
| 57018 | CCNL1 | cyclin L1 |
| 5608 | MAP2K6 | mitogen-activated protein kinase kinase 6 |
| 1026 | CDKN1A | cyclin-dependent kinase inhibitor 1A (p21, Cip1) |
| 783 | CACNB2 | calcium channel, voltage-dependent, beta 2 subunit |
| 1523 | CUTL1 | cut-like 1, CCAAT displacement protein (Drosophila) |
| 10140 | TOB1 | transducer of ERBB2, 1 |
| 23645 | PPP1R15A | protein phosphatase 1, regulatory (inhibitor) subunit 15A |
| 23266 | LPHN2 | latrophilin 2 |
| 3092 | HIP1 | huntingtin interacting protein 1 |
| 5504 | PPP1R2 | protein phosphatase 1, regulatory (inhibitor) subunit 2 |
| 3162 | HMOX1 | heme oxygenase (decycling) 1 |
| 3552 | IL1A | interleukin 1, alpha |
| 3725 | JUN | v-jun sarcoma virus 17 oncogene homolog (avian) |
| 4953 | ODC1 | ornithine decarboxylase 1 |
| 23236 | PLCB1 | phospholipase C, beta 1 (phosphoinositide-specific) |
| 5530 | PPP3CA | protein phosphatase 3 (formerly 2B), catalytic subunit, alpha isoform (calcineurin A alpha) |
| 2263 | FGFR2 | fibroblast growth factor receptor 2 (bacteria-expressed kinase, |
| | | keratinocyte growth factor receptor, craniofacial dysostosis 1, Crouzon syndrome, |
| | | Pfeiffer syndrome, Jackson-Weiss syndrome) |
| 467 | ATF3 | activating transcription factor 3 |
| 900 | CCNG1 | cyclin G1 |
| 5327 | PLAT | plasminogen activator, tissue |
| 23348 | DOCK9 | dedicator of cytokinesis 9 |
| 23412 | COMMD3 | COMM domain containing 3 |
| 54808 | DYM | dymeclin |
| 4781 | NFIB | nuclear factor I/B |
| 3669 | ISG20 | interferon stimulated exonuclease gene 20 kDa |
| 146542 | ZNF688 | zinc finger protein 688 |
| 8870 | IER3 | immediate early response 3 |
| 9518 | GDF15 | growth differentiation factor 15 |
| 220965 | FAM13C1 | family with sequence similarity 13, member C1 |
| 8507 | ENC1 | ectodermal-neural cortex (with BTB-like domain) |
| 26064 | RAI14 | retinoic acid induced 14 |
| 6446 | SGK | serum/glucocorticoid regulated kinase |
| 5607 | MAP2K5 | mitogen-activated protein kinase kinase 5 |
| 54407 | SLC38A2 | solute carrier family 38, member 2 |
| 9538 | EI24 | etoposide induced 2.4 mRNA |
| 80219 | COQ10B | coenzyme Q10 homolog B (yeast) |
| 23331 | RP3-477H23.1 | KIAA1043 protein |
| 7405 | UVRAG | UV radiation resistance associated gene |
| 9873 | FCHSD2 | FCH and double SH3 domains 2 |
| 1793 | DOCK1 | dedicator of cytokinesis 1 |
| 9693 | RAPGEF2 | Rap guanine nucleotide exchange factor (GEF) 2 |
| 54914 | KIAA1797 | KIAA1797 |
| 4193 | MDM2 | Mdm2, transformed 3T3 cell double minute 2, p53 binding protein (mouse) |
| 157680 | VPS13B | vacuolar protein sorting 13B (yeast) |
| 1730 | DIAPH2 | diaphanous homolog 2 (Drosophila) |
| 6840 | SVIL | supervillin |
| 29950 | SERTAD1 | SERTA domain containing 1 |
| 90865 | C9orf26 | chromosome 9 open reading frame 26 (NF-HEV) |
| 987 | LRBA | LPS-responsive vesicle trafficking, beach and anchor containing |
| 79899 | FLJ14213 | hypothetical protein FLJ14213 |
| 5218 | PFTK1 | PFTAIRE protein kinase 1 |
| 23612 | PHLDA3 | pleckstrin homology-like domain, family A, member 3 |
| 2530 | FUT8 | fucosyltransferase 8 (alpha (1,6) fucosyltransferase) |
| 10614 | HEXIM1 | hexamethylene bis-acetamide inducible 1 |
| 51278 | IER5 | immediate early response 5 |
| 23345 | SYNE1 | spectrin repeat containing, nuclear envelope 1 |
| 54622 | ARL15 | ADP-ribosylation factor-like 15 |
| 9611 | NCOR1 | nuclear receptor co-repressor 1 |
| 1316 | KLF6 | Kruppel-like factor 6 |
| 9510 | ADAMTS1 | ADAM metallopeptidase with thrombospondin type 1 motif, 1 |
| 468 | ATF4 | activating transcription factor 4 (tax-responsive enhancer element B67) |
| 1488 | CTBP2 | C-terminal binding protein 2 |
| 6574 | SLC20A1 | solute carrier family 20 (phosphate transporter), member 1 |
| 56288 | PARD3 | par-3 partitioning defective 3 homolog (C. elegans) |

human subset 2 (d)
1020; 1027; 1029; 1051; 1054; 1387; 1457; 1460; 1613; 1647; 1649; 1655; 2122; 2275; 2308; 2309; 2580; 2963; 3065; 3091; 3146; 3556; 3725; 3726; 3727; 4087; 4088;
4089; 4094; 4193; 4194; 4286; 4303; 4582; 4609; 4616; 4654; 4666; 4670; 4692; 4779; 4780; 4784; 4790; 4794; 4842; 5111; 5292; 5300; 5328; 5335; 5371; 5411; 5425;
5432; 5449; 5469; 5524; 5587; 5590; 5594; 5595; 5599; 5600; 5601; 5602; 5684; 5716; 5879; 5891; 5925; 5934; 5970; 6125; 6135; 6142; 6284; 6304; 6418; 6497; 6597;
6602; 6647; 6667; 6678; 6688; 6689; 6772; 6774; 6775; 6872; 6885; 6908; 6935; 6942; 7041; 7050; 7083; 7123; 7153; 7157; 7161; 7169; 7251; 7329; 7341; 7421; 7528;
7533; 7709; 7850; 7874; 8061; 8204; 8290; 8317; 8440; 8535; 8550; 8554; 8570; 8648; 8650; 8841; 8850; 8878; 8900; 9026; 9063; 9188; 9261; 9314; 9325; 9351; 9412;
9495; 9564; 9568; 9584; 9586; 9672; 9759; 9844; 9950; 9975; 10009; 10014; 10163; 10456; 10514; 10524; 10538; 10746; 10771; 10912; 10970; 10987; 11279; 22853;
23013; 23176; 23210; 23327; 23411; 23414; 23493; 23523; 23741; 23770; 25792; 25920; 25942; 27085; 27185; 28954; 29984; 50855; 51177; 51274; 51517; 51776; 51778;
53353; 54822; 54861; 55081; 55294; 55553; 55690; 56288; 56647; 57761; 60528; 63916; 64219; 64326; 79718; 80746; 84687; 91977; 121504; 122953; 161882; 200186;
255488; 284217
human subset 3 (d)
2; 12; 25; 60; 70; 100; 142; 160; 162; 176; 182; 196; 207; 210; 211; 239; 302; 328; 332; 355; 356; 367; 408; 409; 440; 466; 467; 468; 472; 545; 571; 572; 573; 580; 581; 595;
596; 598; 602; 604; 606; 627; 639; 644; 650; 660; 672; 673; 694; 710; 775; 776; 781; 782; 784; 785; 801; 811; 819; 836; 860; 861; 862; 864; 867; 891; 892; 894; 896; 898;
901; 904; 905; 920; 943; 960; 983; 985; 993; 998; 999; 1000; 1001; 1017; 1019; 1020; 1021; 1025; 1026; 1027; 1028; 1029; 1030; 1031; 1032; 1033; 1045; 1050; 1051;
1053; 1063; 1105; 1147; 1191; 1212; 1213; 1230; 1231; 1361; 1385; 1386; 1387; 1398; 1401; 1432; 1435; 1457; 1459; 1460; 1462; 1487; 1491; 1499; 1508; 1535; 1571;
1588; 1613; 1639; 1642; 1647; 1649; 1655; 1660; 1673; 1718; 1728; 1729; 1794; 1803; 1820; 1827; 1831; 1869; 1874; 1950; 1956; 1958; 1965; 1977; 1993; 1994; 2002;
2005; 2010; 2033; 2052; 2064; 2066; 2078; 2099; 2100; 2113; 2114; 2122; 2147; 2149; 2152; 2159; 2160; 2192; 2237; 2243; 2245; 2246; 2247; 2250; 2252; 2254; 2255;
2260; 2261; 2264; 2275; 2280; 2309; 2321; 2335; 2353; 2354; 2355; 2357; 2495; 2516; 2528; 2534; 2550; 2580; 2623; 2624; 2649; 2670; 2671; 2672; 2688; 2689; 2720,
2735; 2776; 2804; 2874; 2885; 2902; 2908; 2932; 2961; 2963; 3054; 3064; 3065; 3066; 3074; 3082; 3091; 3093; 3146; 3159; 3162; 3163; 3172; 3182; 3184; 3190; 3265,
3280; 3303; 3308; 3315; 3375; 3383; 3397; 3428; 3429; 3458; 3479; 3480; 3482; 3485; 3488; 3516; 3551; 3553; 3556; 3557; 3558; 3565; 3566; 3567; 3569; 3576; 3577;
3596; 3620; 3621; 3630; 3655; 3859; 3662; 3678; 3688; 3693; 3713; 3717; 3725; 3726; 3727; 3752; 3753; 3791; 3818; 3838; 3845; 3856; 3868; 3909; 3958; 3964; 4000;
4035; 4043; 4049; 4054; 4067; 4082; 4084; 4087; 4088; 4089; 4092; 4094; 4129; 4144; 4152; 4170; 4193; 4194; 4204; 4208; 4214; 4215; 4216; 4233; 4267; 4297; 4312;
4313; 4318; 4436; 4507; 4583; 4593; 4602; 4609; 4613; 4616; 4654; 4655; 4666; 4670; 4683; 4690; 4692; 4734; 4751; 4772; 4773; 4774; 4775; 4776; 4779; 4780; 4782;
4784; 4790; 4791; 4792; 4794; 4842; 4843; 4851; 4853; 4904; 4914; 4915; 4946; 4947; 5008; 5027; 5045; 5052; 5054; 5055; 5058; 5074; 5093; 5098; 5104; 5111; 5134;
5163; 5170; 5241; 5245; 5274; 5292; 5300; 5328; 5329; 5330; 5331; 5335; 5340; 5347; 5364; 5371; 5373; 5411; 5421; 5422; 5432; 5449; 5451; 5465; 5467; 5468; 5478;
5499; 5501; 5505; 5521; 5524; 5532; 5533; 5534; 5536; 5540; 5578; 5579; 5580; 5581; 5583; 5584; 5585; 5587; 5590; 5591; 5594; 5595; 5597; 5598; 5599; 5600; 5601;
5602; 5603; 5604; 5606; 5609; 5621; 5657; 5684; 5716; 5724; 5728; 5730; 5742; 5743; 5747; 5757; 5764; 5781; 5879; 5891; 5894; 5903; 5914; 5916; 5925; 5932; 5933;
5934; 5966; 5970; 5981; 5999; 6101; 6125; 6135; 6142; 6195; 6275; 6284; 6294; 6297; 6300; 6302; 6304; 6337; 6338; 6340; 6347; 6348; 6364; 6387; 6401; 6403; 6416;
6418; 6419; 6427; 6428; 6478; 6497; 6507; 6512; 6513; 6523; 6575; 6597; 6598; 6602; 6612; 6613; 6647; 6654; 6667; 6670; 6688; 6689; 6714; 6722; 6772; 6774; 6775;
6776; 6872; 6885; 6907; 6908; 6915; 6929; 6935; 6942; 7015; 7020; 7027; 7040; 7041; 7046; 7050; 7056; 7073; 7083; 7094; 7114; 7123; 7124; 7153; 7157; 7159; 7161;
7186; 7187; 7224; 7251; 7252; 7291; 7295; 7296; 7322; 7323; 7329; 7341; 7371; 7412; 7421; 7422; 7443; 7450; 7464; 7486; 7528; 7530; 7533; 7704; 7709; 7809; 7832;
7850; 7874; 7917; 8061; 8091; 8202; 8204; 8290; 8295; 8315; 8440; 8451; 8467; 8535; 8554; 8570; 8573; 8615; 8626; 8648; 8650; 8701; 8743; 8819; 8820; 8841; 8850;
8899; 8900; 8941; 9021; 9026; 9134; 9173; 9184; 9188; 9261; 9314; 9344; 9349; 9351; 9368; 9401; 9467; 9495; 9507; 9508; 9509; 9518; 9540; 9564; 9568; 9572; 9584;
9586; 9612; 9672; 9682; 9759; 9771; 9836; 9844; 9950; 9975; 9978; 10009; 10014; 10110; 10155; 10163; 10231; 10298; 10397; 10413; 10456; 10514; 10524; 10538;
10746; 10766; 10771; 10783; 10788; 10797; 10848; 10850; 10912; 10919; 10970; 10987; 10991; 11018; 11095; 11127; 11177; 11200; 11279; 22808; 22809; 22853; 22859;
23054; 23113; 23176; 23210; 23224; 23230; 23236; 23284; 23327; 23365; 23368; 23414; 23426; 23462; 23493; 23522; 23523; 23678; 23741; 23770; 25780; 25792; 25920;
25942; 26574; 26960; 27085; 27250; 28954; 28996; 29127; 29843; 29984; 30819; 50848; 50855; 51177; 51274; 51517; 51776; 53353; 54822; 54832; 54861; 55070; 55187;
55294; 55509; 55553; 55663; 55690; 55717; 56288; 56302; 56647; 56924; 57447; 57551; 57761; 60528; 63916; 63970; 64219; 64326; 64395; 64407; 64975; 79042; 79718;
80263; 80316; 80746; 80764; 83660; 83737; 84124; 84168; 84687; 89848; 116461; 117583; 121504; 122953; 124790; 125050; 153743; 161882; 171017; 255488; 283989;
284217; 442219; 544618
chimpanzee subset 1 (d)
468310; 472471; 468406; 456191; 456974; 460949; 470195; 470485; 456893; 459027; 462243; 472748; 452635; 450347; 455412; 464995; 462547; 468770; 471519; 461932; 463011; 451851; 466842; 459851; 451430;
451409; 472904; 450383; 456037; 471319; 463522; 457620; 449506; 454746; 457563; 463081; 456597; 461876; 468370; 450269; 470057; 468334; 459498
chimpanzee subset 2 (d)
463891; 465021; 469969; 468818; 467891; 455594; 456929; 452017; 456768; 461048; 464259; 452958; 452518; 460935; 456893; 468739; 455407; 468537; 457658;
473659; 451057; 452434; 455671; 453273; 455185; 459769; 468742; 461406; 462730; 467139; 458081; 462656; 453736; 452875; 472654; 454119; 470861; 454629;
464784; 452834; 454037; 450445; 465803; 463253; 453176; 452715; 467978; 466665; 457025; 464992; 457324; 460213; 451889; 451939; 471709; 470721; 459834;
465701; 462888; 450387; 458876; 467962; 455312; 468342; 460314; 465078; 451065; 467865; 459882; 451858; 458778; 470455; 453907; 469380; 459472; 467715;
455640; 453014; 460219; 467260; 455403; 450500; 464640; 453505; 465356; 467480; 454246; 463318; 453119; 460230; 465831; 454721; 456675; 457347; 451329;
450253; 464585; 466759; 465661; 472454; 456468; 462057; 468338; 455443; 466088; 464332; 472118; 458709; 455862; 464902; 473285; 453759; 464361; 457820;
458153; 451358; 454171; 457268; 461165; 460351; 459745; 453429; 460301; 470877; 466449; 466669; 450814; 469852; 450109; 458299; 457541; 471003; 453048;
469502; 462463
chimpanzee subset 3 (d)
453133; 464802; 450133; 453307; 457785; 468956; 458967; 453629; 453202; 464675; 460416; 455242; 453487; 465200; 466149; 469584; 465680; 454450; 463556;
452429; 468334; 460745; 458500; 451291; 465041; 470638; 458156; 462904; 463366; 458090; 449497; 463781; 452121; 461302; 454624; 451867; 459660; 453100;
455756; 472017; 451690; 461835; 462689; 455914; 461229; 470546; 450124; 457960; 450481; 460341; 456609; 468509; 452025; 463891; 463534; 465021; 455931;
469969; 450673; 464075; 462288; 452582; 467891; 457428; 462643; 457127; 461766; 456939; 463993; 468068; 450857; 453433; 455594; 459332; 451234; 456929;
452017; 457568; 468012; 462144; 471731; 459689; 458184; 454158; 462097; 452980; 461391; 465023; 455668; 473591; 465938; 458862; 457777; 454636; 459919;
458548; 463079; 451158; 471510; 457038; 452679; 458908; 451242; 461559; 462158; 471190; 467226; 471553; 461067; 462301; 456768; 453047; 456127; 459115;
456254; 461048; 465619; 460672; 464725; 454741; 460254; 460624; 455228; 471681; 470895; 464098; 464259; 462950; 461807; 463503; 452958; 461178; 452518;
462624; 470195; 453880; 458269; 461216; 464554; 466302; 460942; 462060; 463488; 450196; 469912; 457424; 453250; 449517; 453676; 463112; 459932; 459933;
461152; 472749; 450200; 460935; 459501; 461472; 449565; 454005; 463288; 471205; 470645; 464134; 450954; 459742; 462386; 451958; 450396; 456893; 468739;
461315; 471375; 454841; 473387; 455042; 469155; 457385; 452006; 461086; 472771; 470395; 455407; 468537; 455411; 465588; 455418; 457274; 457658; 465936;
461760; 471543; 468286; 463119; 463671; 465979; 451587; 451510; 454094; 459215; 464653; 463016; 459043; 451057; 452434; 455671; 464278; 453273; 467689;
455485; 469972; 452821; 455185; 459769; 455583; 468742; 461406; 467426; 462730; 467139; 455026; 470311; 452318; 453652; 456537; 472467; 455460; 451439;
452094; 459300; 453132; 458081; 465124; 459743; 451502; 455205; 460834; 462656; 450103; 453991; 460344; 453736; 453905; 452875; 465543; 454119; 470861;
460178; 462170; 464784; 450898; 456150; 466064; 459203; 450143; 452834; 464165; 454037; 450445; 462644; 453526; 454515; 458076; 465803; 469249; 466142;
464713; 464422; 460522; 463759; 467135; 463253; 453176; 460182; 459477; 451935; 452715; 455333; 467978; 470380; 466665; 461174; 457470; 472766; 457025;
464992; 457320; 457324; 452024; 460213; 451776; 453984; 459991; 450191; 455065; 470738; 460781; 461918; 469329; 458779; 464151; 458702; 451889; 458605;
463937; 459171; 451939; 459750; 458229; 462709; 470721; 459834; 454681; 465701; 462888; 468667; 468650; 450387; 458876; 471443; 456060; 467962; 472992;
455312; 469898; 450776; 468342; 465080; 460314; 464876; 453178; 451065; 450779; 467112; 462108; 467865; 459882; 457479; 451858; 453148; 472732; 458778;
451557; 469648; 470455; 453907; 462560; 458308; 463564; 459472; 452694; 461517; 467715; 455640; 461243; 453014; 472305; 460852; 471351; 460219; 462406;
467260; 459953; 464292; 459462; 450500; 464640; 454619; 454865; 464469; 467480; 457453; 461267; 462325; 468770; 454246; 454644; 463318; 467150; 456829;
453119; 460230; 458860; 465831; 454721; 456353; 456675; 462742; 451506; 457347; 451329; 458865; 450253; 473058; 459331; 464585; 462572; 466759; 455719;
466852; 450168; 452855; 458733; 465661; 470938; 472454; 463192; 462057; 468338; 455443; 451618; 453193; 464332; 472118; 450534; 458709; 464217; 455862;
470348; 464902; 473285; 453759; 454601; 464361; 450735; 458153; 451887; 451862; 451358; 450692; 469555; 454171; 457268; 461165; 460351; 459745; 453429;
453491; 460301; 457969; 466449; 466669; 450784; 450814; 453326; 469852; 450109; 458299; 457541; 457592; 456281; 471003; 457158; 473574; 458191; 463573;
462147; 457576; 453048; 471716; 462463; 472042

TABLE 6B
toxicology biomarkers (non-primate)

dog subset 1 (j)
479417; 481425; 482143; 478546; 478546; 481031; 490013; 483332; 477887; 489173; 484963; 485332; 486666; 487374; 487554; 488150; 489458; 476629
dog subset 2 (j)
488833; 403934; 489971; 476636; 479483; 477263; 479210; 403955; 484648; 488568; 480042; 476462; 480815; 480525; 403869; 489620; 485016; 484056; 490050;
477761; 479632; 607232; 608113; 476419; 482362; 479777; 474702
dog subset 3 (j)
479476; 477753; 489433; 442968; 403954; 475924; 611541; 488833; 449028; 403416; 487097; 403567; 489369; 488997; 610456; 490924; 403934; 491325; 474890;
479866; 477185; 478108; 474844; 612240; 477032; 479353; 610935; 486025; 490792; 474485; 403819; 403818; 483312; 487309; 475035; 482371; 482372; 607052;
403735; 610255; 442990; 609429; 611584; 480488; 474619; 483758; 485023; 481556; 491946; 403924; 474772; 608738; 485930; 476636; 474486; 479483; 494005;
477575; 442942; 479210; 403955; 484648; 488568; 479446; 483729; 476664; 480042; 403647; 609858; 487293; 490682; 488449; 490967; 476462; 490387; 474771;
480525; 403869; 489620; 476714; 485016; 484056; 487096; 484820; 485915; 487352; 476780; 475589; 490050; 477761; 475827; 607247; 490739; 609018; 607232;
483780; 488741; 483339; 479880; 608113; 486338; 478603; 480695; 612892; 476419; 403403; 487130; 478309; 609974; 491088; 477152; 484047; 607836; 479229;
479777; 474702; 482645; 489174; 489175
rat subset 1 (j)
116781; 315606; 297392; 305307; 288165; 288165; 313449; 302915; 303690; 122772; 155205; 298696; 362456; 83510; 81639; 301004
rat subset 2 (j)
305341; 64465; 25515; 316009; 363633; 24697; 25532; 363875; 366957; 24703; 304794; 304850; 308472; 373541; 24842; 362675; 25156; 54319; 287101; 309523; 299173; 298508; 362093; 171071; 310194; 309331
rat subset 3 (j)
287454; 25290; 78971; 25385; 117273; 360678; 360500; 305341; 58919; 24224; 307151; 25402; 500985; 54237; 307511; 360643; 64465; 362110; 114851; 497663;
116549; 307641; 29382; 84353; 79128; 24314; 170915; 314322; 25019; 25183; 29662; 81504; 297893; 84577; 500126; 297099; 368178; 502373; 24482; 116553; 24516;
363220; 29417; 301119; 305152; 24577; 298075; 311658; 316009; 300089; 363633; 170538; 116590; 29527; 25532; 363875; 366957; 24703; 304794; 315655; 309165;
304850; 29517; 81921; 140941; 140593; 24784; 85384; 25124; 25125; 308472; 313121; 297758; 289316; 25566; 24842; 362675; 298706; 25156; 54319; 361285; 306347;
84584; 297867; 89816; 290829; 287101; 309523; 295284; 64845; 25514; 298508; 497739; 83788; 287113; 362093; 114212; 124461; 288040; 192348; 298316; 171071;
361401; 361271; 58814; 362547; 303456; 311468; 497729; 361176; 310194; 309331; 295171; 497667; 303148
mouse subset 1 (j)
mouse subset 2 (j)
74734; 12540; 18817; 235611; 20020; 19246; 19341; 19353; 19354; 110157; 213464; 19821; 20924; 545600; 21973; 22059; 22062; 22324; 22350; 268930; 68533; 56031; 140858; 17872; 56524; 67773; 109113
mouse subset 3 (j)
11684; 11689; 11796; 14103; 11848; 192662; 14570; 74734; 12443; 12043; 12151; 12367; 12402; 12534; 12532; 217232; 12540; 107951; 12575; 12914; 12995; 13000;
13001; 13016; 12387; 13132; 18104; 328572; 14255; 14281; 14375; 14567; 14569; 14784; 433759; 15182; 15404; 15405; 15426; 15461; 16000; 16163; 16476; 16764;
17283; 64144; 17355; 70122; 54601; 17869; 433702; 18019; 235611; 29858; 20020; 18753; 26413; 19225; 19341; 19353; 19354; 110157; 213464; 19684; 19697; 19704;
19821; 20393; 20405; 20408; 20437; 20439; 105243; 20663; 20846; 20848; 20924; 26409; 21749; 320202; 21916; 21973; 22059; 22062; 54721; 22324; 22350; 17354;
13716; 17979; 93760; 233489; 23808; 268930; 60365; 11308; 268566; 18777; 56031; 53319; 20352; 53860; 140858; 50883; 23970; 68092; 14269; 56526; 17872; 192654;
326622; 50876; 320438; 217109; 75812; 239985; 67031; 52463; 67773; 109113; 27059; 246049; 215332
dog subset 1 (cd)
403800; 608289; 442987; 490461; 477955; 489210; 611782; 483312; 608325; 487381; 488036; 610454; 476216
dog subset 2 (cd)
487382; 477707; 481131; 475537; 609920; 485158; 491942; 442955; 488508; 486400; 491021; 491286; 489168; 481938; 490012; 479455; 479074; 474964; 403735; 487739; 403989; 403439; 403785; 478800;
486148; 403789; 477956; 483318; 489159; 442952; 484185; 485427; 403717; 488635; 403760; 482154; 403811; 478151; 478781; 478275; 488504; 487325; 403438; 444854; 448784; 481884; 477498; 490455;
612616; 490404; 492123; 607885; 487221; 403911; 442985; 478938; 487235; 609956; 485874; 480459; 485783; 481014; 475108; 477263; 490563; 486717; 477488; 483277; 477176; 490255; 609852; 609725;
479158; 403494; 490463; 607023; 403522; 480080; 609265; 488797; 481619; 490439; 485837; 483042; 490682; 485864; 490968; 484196; 474759; 403634; 403869; 476924; 485377; 481716; 485016;
608060; 484056; 480435; 491867; 607740; 612006; 481713; 492059; 480887; 488125; 480543; 490006; 485548; 611262; 490434; 484052; 479428; 475024; 475213; 478299; 480450; 491756; 488556;
480194; 609976; 480354; 479648; 479515; 486242; 484169; 487132; 485167; 475217; 491093; 487343; 488025; 483838; 485504; 474362; 483386; 479921; 483060; 487316; 481794; 474444; 485195; 484527;
477133; 477219; 479605; 476526; 477369; 474928; 612792; 483124; 607947; 611919; 486814; 484500; 612797; 484776; 482086; 478300; 481343; 484518; 610895; 477371; 480668; 475740; 474913; 490859;
479211; 611101; 488813; 480244; 478760; 476455; 481816; 488859; 481859
dog subset 3 (cd)
491292; 475923; 608134; 479191; 477236; 403817; 403910; 483701; 490878; 474808; 476600; 477753; 483870; 403435; 403407; 442968; 475176; 489461; 476535; 403731; 484490; 487718; 449028; 488770;
488124; 607482; 475867; 477226; 478194; 487382; 477707; 481722; 489221; 612941; 403474; 489369; 487965; 608847; 484610; 488609; 607448; 611695; 485430; 476485; 403646; 484382; 474391; 481595;
442858; 489775; 481131; 475537; 609920; 474890; 403429; 481563; 611216; 484790; 403826; 478835; 403840; 403582; 487950; 609516; 607922; 478806; 486977; 608125; 403856; 489188; 607605; 487649;
477185; 477032; 489664; 415128; 479353; 476623; 476132; 483106; 485158; 491942; 489462; 491319; 487984; 486276; 489117; 478881; 475115; 403657; 404306; 487421; 475590; 610283; 442955; 480113;
484943; 403883; 488508; 486102; 475582; 488808; 415125; 477295; 403727; 486025; 481464; 481070; 478232; 475032; 483808; 486400; 480808; 491021; 478770; 489168; 481938; 490012; 479455; 553102;
479074; 480348; 479864; 485884; 474964; 403735; 403612; 478854; 609748; 612371; 403801; 487739; 442951; 403989; 403785; 489957; 403790; 403545; 403850; 484828; 442990; 481885; 475574; 484990;
478800; 486148; 489208; 403789; 488429; 486493; 489905; 477956; 403788; 483318; 489159; 442952; 484185; 609429; 485427; 403995; 403717; 488635; 403760; 482154; 403811; 478151; 489126; 403616;
478781; 488643; 484932; 478275; 488504; 487325; 476160; 403438; 479417; 444854; 403768; 448784; 403924; 481884; 607700; 480036; 485677; 477535; 478813; 442859; 477498; 487441; 490455; 612616;
490404; 484147; 492123; 607885; 403560; 403911; 442860; 442985; 482310; 475823; 611916; 478938; 487235; 609956; 485874; 489692; 480344; 403556; 480459; 494005; 477575; 486758; 477746; 481014;
403832; 475108; 477263; 490563; 486717; 477488; 483277; 477176; 490255; 609852; 475389; 404008; 479997; 484648; 475875; 483881; 477045; 479158; 476915; 403494; 612326; 490460; 490463; 475852;
607023; 403981; 480600; 403522; 480080; 484763; 609265; 488797; 481619; 490439; 482437; 403673; 485837; 483042; 490682; 486507; 488405; 485864; 488449; 490968; 485402; 484196; 403412; 403998;
489932; 487486; 474759; 403922; 403634; 403869; 476924; 403575; 481716; 485016; 608060; 403802; 484056; 491867; 488409; 607740; 490323; 484762; 612006; 475424; 492059; 480887; 488125; 480543;
485548; 488487; 484052; 479428; 475024; 475213; 478299; 480450; 491756; 481241; 480354; 479648; 479515; 486242; 484169; 487132; 480331; 485167; 475217; 403702; 481396; 487343; 488025; 483838;
485504; 476150; 474362; 483386; 483060; 475930; 482271; 487316; 482135; 481794; 474444; 485195; 490548; 490685; 484527; 607929; 485404; 480695; 482513; 477369; 482853; 483124; 607947; 611919;
486814; 479812; 484500; 612797; 481337; 484776; 478300; 481343; 484518; 480054; 477371; 475740; 474913; 490859; 479211; 611101; 488813; 489289; 480244; 478138; 477128; 481816; 488859; 481859
rat subset 1 (cd)
rat subset 2 (cd)
362634; 312705; 94201; 140908; 114483; 64053; 58948; 313052; 59323; 84427; 54257; 24409; 66012; 29424; 502373; 171016; 360697; 116562; 287287; 54259; 114517;
25467; 24511; 25724; 363577; 84598; 24514; 25469; 29712; 29718; 117052; 64030; 313050; 25152; 25595; 297894; 300999; 315840; 24603; 25339; 59109; 317576;
29431; 24627; 497724; 289021; 29741; 25738; 24686; 25614; 24697; 117063; 116689; 25622; 360370; 24699; 114767; 362161; 25676; 24716; 94195; 60352; 81780;
29259; 303468; 305450; 362513; 85385; 362242; 313845; 85384; 83805; 24918; 25155; 24949; 57027; 313121; 89804; 313494; 25625; 24842; 116646; 25009; 25156;
296603; 54319; 314442; 317371; 317676; 29737; 294250; 309072; 315350; 29647; 246334; 286975; 252971; 29376; 246756; 57025; 84018; 500989; 294568; 296121;
64520; 363466; 116681; 65040; 56725; 25414; 54299; 360801; 83585; 291309; 84477; 114485; 287622; 303882; 294981; 306141; 116633; 29751; 295378; 65037; 117268;
316213; 314850; 117185; 292763; 362229; 286930; 117548; 312652; 305967; 497836; 295338; 292846; 81511; 500449; 499356; 361526; 84357; 287659; 301009; 114858;
300045; 499884; 498232; 301346; 292759; 290326; 296562; 363194; 79146; 287126; 305391; 85265; 311055; 292735; 316233; 305408; 170843
rat subset 3 (cd)
311860; 29437; 303742; 291245; 24165; 25026; 24176; 25238; 24185; 25374; 25290; 56611; 54226; 25385; 29183; 24210; 24215; 29600; 83625; 304127; 58919; 170929;
303836; 116599; 296321; 298566; 362634; 312705; 117517; 25400; 24248; 500985; 171136; 25729; 25109; 497761; 309217; 308501; 25660; 29185; 25406; 29186;
313498; 83502; 307618; 94201; 140908; 114483; 114851; 83571; 246060; 361516; 60463; 25412; 84032; 367313; 304021; 499431; 81646; 81648; 54245; 287942; 81649;
307403; 298518; 315707; 116549; 307505; 84353; 79129; 25086; 79128; 114489; 116483; 29167; 317189; 64053; 58948; 497670; 140694; 29238; 114856; 363241; 25313;
24329; 366492; 116636; 60465; 313052; 25315; 24336; 24337; 59323; 50646; 84489; 25022; 84482; 294515; 54251; 497666; 24948; 56718; 25150; 361388; 171104;
84427; 497769; 54257; 24407; 24409; 66012; 25202; 29424; 29560; 79239; 25735; 502373; 171016; 294254; 497838; 25462; 313641; 298205; 25712; 360697; 25718;
116562; 287287; 25084; 24497; 25205; 116558; 171333; 116553; 66014; 66029; 54259; 24954; 114517; 25467; 170921; 360606; 311144; 315346; 308995; 24511; 25724;
363577; 84598; 24514; 24516; 25469; 25470; 29712; 29718; 117052; 64030; 24525; 313050; 155918; 25608; 289227; 361378; 25152; 25595; 297894; 315606; 300999;
24577; 315840; 114553; 364018; 300955; 25744; 83619; 81736; 24603; 25339; 59109; 25054; 317576; 29431; 362282; 25267; 497724; 89813; 65144; 94203; 289021;
29741; 25738; 29337; 24666; 25636; 170538; 116590; 50689; 50557; 25614; 24697; 117063; 116689; 25622; 360370; 24699; 114767; 360406; 64633; 499636; 24703;
81757; 116546; 24706; 25676; 24708; 24716; 114216; 94195; 445415; 60352; 287562; 81780; 29259; 315996; 303468; 305450; 362513; 85385; 24778; 25139; 362242;
313845; 85384; 24790; 367846; 83805; 25124; 24918; 25559; 25155; 24949; 57027; 89804; 301965; 59086; 84574; 313494; 24835; 25625; 24842; 116646; 25708; 25009;
25156; 296603; 83785; 54319; 317371; 117538; 317676; 301348; 170672; 363142; 29737; 310678; 314764; 315350; 29647; 246334; 286975; 29376; 81527; 84018;
500989; 294568; 296121; 64520; 363466; 366962; 25414; 54299; 360801; 83585; 291309; 289997; 84477; 114485; 81732; 289820; 303882; 294981; 306141; 311030;
116633; 29751; 65037; 296787; 29374; 117268; 289596; 316213; 314850; 117185; 84014; 286988; 503027; 292763; 363140; 124461; 192348; 313819; 305967; 306543;
295338; 292846; 81511; 500449; 499356; 25435; 361526; 84357; 287659; 316344; 301009; 114858; 499884; 498232; 301346; 292759; 304887; 290326; 363194; 79146;
287126; 305391; 315546; 85265; 114017; 316233; 305408; 170843; 316758
mouse subset 1 (cd)
17748; 230899; 15368; 20201; 18186; 12444; 14784; 13627; 13629; 12260; 13383; 12508; 15979
mouse subset 2 (cd)
12262; 50909; 12567; 12568; 12571; 23859; 53310; 269587; 13869; 14598; 14786; 107746; 14799; 14806; 14811; 234889; 15194; 15381; 15461; 94175; 15980; 16183;
16185; 16189; 16331; 16403; 16367; 16399; 16412; 192897; 16428; 16451; 16452; 16492; 16518; 16513; 16515; 16542; 16590; 16818; 14725; 17756; 17180; 17295;
19882; 17829; 17920; 18125; 18160; 18162; 18211; 320634; 18479; 238871; 18588; 18596; 240752; 18708; 18709; 18803; 19084; 19122; 53380; 14083; 19246; 19255;
15170; 19247; 19248; 19262; 19264; 19267; 19288; 19400; 218397; 19713; 20202; 29857; 20304; 20343; 20391; 24055; 230126; 20416; 20648; 20662; 20663; 20779;
20850; 20963; 20964; 11491; 26409; 21687; 21894; 21937; 22059; 22070; 22141; 22321; 22324; 22325; 22350; 22375; 22376; 56743; 16497; 53761; 11489; 16370; 12361;
22061; 78473; 12703; 384783; 71609; 11490; 20975; 22687; 83767; 73178; 84092; 116870; 194590; 244310; 224997; 56724; 12927; 20185; 20602; 14544; 98910; 14389;
23857; 16923; 242687; 51789; 24063; 24064; 56399; 20346; 57257; 17289; 20218; 107971; 327826; 50877; 26411; 24128; 228836; 16561; 67784; 16554; 12488; 20399;
19260; 83382; 16797; 54390; 17060; 55942; 58194; 192157; 80987; 20418; 109075; 58800; 240754; 20353; 71984; 94212; 52033; 18146; 116871; 72322; 380785; 69581;
79044; 66999; 16475; 66860; 192192; 74094; 210710; 70693; 170771
mouse subset 3 (cd)
11350; 11459; 71885; 11472; 11486; 11535; 11555; 110355; 11651; 17025; 11655; 11689; 30948; 12306; 11820; 14103; 11839; 11889; 11941; 381290; 26362; 12013;
12443; 12047; 12053; 110279; 109778; 329547; 12259; 12262; 50909; 12268; 12322; 12359; 12402; 208650; 12445; 12447; 12480; 12481; 12527; 12482; 12483; 12487;
12493; 12505; 626721; 12517; 12550; 12562; 12567; 12568; 12571; 12575; 12576; 12577; 12581; 232925; 12772; 12824; 12825; 94216; 12835; 12837; 12817; 12912;
11909; 12918; 12928; 12929; 26416; 12978; 12986; 12988; 12995; 12385; 12387; 13057; 13106; 13132; 13138; 226414; 13191; 54004; 23859; 53310; 13385; 13429;
13490; 14357; 19252; 55949; 66656; 13645; 13649; 13836; 13685; 13043; 269587; 13849; 13857; 13866; 13869; 19229; 14182; 14184; 14183; 56458; 56484; 14254;
14255; 14257; 14265; 56717; 14360; 14388; 14598; 76854; 14786; 50915; 14799; 14806; 14809; 14811; 234889; 54195; 15194; 15251; 15369; 15378; 15381; 15461;
94175; 193740; 432551; 628438; 15974; 15978; 15980; 16001; 16183; 16189; 16190; 16191; 16195; 16198; 16161; 16163; 63859; 15930; 16331; 16337; 16403; 16367;
16398; 16399; 16400; 16401; 16402; 16408; 16412; 16416; 192897; 16428; 16451; 16452; 16476; 16492; 16493; 16518; 16513; 16515; 16542; 16590; 16653; 16818;
16822; 16846; 14725; 17085; 17159; 17756; 17180; 17196; 17295; 214162; 19882; 17750; 17829; 17869; 17920; 17969; 17970; 17973; 18016; 18024; 18033; 18125;
18160; 18162; 18211; 18212; 320634; 18479; 18534; 18588; 18595; 18596; 27273; 59020; 18654; 240752; 18708; 18709; 18803; 234779; 19042; 18747; 19084; 18753;
26413; 26417; 26419; 53380; 19211; 14083; 19246; 19255; 15170; 19247; 19248; 19262; 19264; 19267; 19268; 19288; 271457; 98710; 110157; 56044; 64143; 218772;
218397; 19645; 19713; 20197; 20202; 29857; 20293; 20304; 20343; 20350; 20391; 24055; 230126; 20416; 20525; 20528; 20648; 20662; 20663; 20683; 20687; 20779;
20846; 20850; 20927; 20963; 20964; 11491; 21687; 21752; 21803; 21804; 21825; 21894; 21926; 21937; 22059; 22070; 22229; 22321; 22324; 22325; 22339; 22340; 22350;
22376; 215280; 56743; 22637; 170484; 20347; 16497; 382523; 216238; 16370; 12361; 22061; 78473; 384783; 18187; 20975; 22687; 83767; 73178; 84092; 116870;
194590; 17444; 12927; 20185; 20602; 14544; 98910; 218977; 14389; 23857; 16923; 74117; 66713; 242687; 51789; 24063; 24064; 56324; 56399; 20346; 17289; 20348;
12464; 20218; 53419; 107971; 327826; 50877; 19765; 108767; 399510; 26411; 56289; 246694; 23970; 14269; 17776; 16554; 78560; 19260; 83382; 16797; 54390; 17060;
13631; 55942; 58194; 192157; 56210; 80987; 20418; 58800; 240754; 20353; 71984; 78255; 52033; 116871; 72322; 380785; 69581; 79044; 66999; 16475; 28030; 74094;
70693; 170771; 16772
dog subset 1 (cpm)
607526; 484957; 489430; 607514; 610296; 480411; 488210; 610120; 610819; 482044; 488660; 480322; 483841; 490823; 477284; 475772; 485848; 479526; 608025;
490864; 484822; 611442; 403649; 476117; 491194; 484867; 486511; 475840; 478470; 403767; 403848; 481713; 489584; 490730; 493997; 403802; 488605; 487486;
474355; 403998; 484064; 485402; 481228; 486138; 477542; 403994; 480080; 483617; 490299; 474867; 478013; 609421; 478980; 475235; 403855; 475234; 403602;
403426; 482840; 607205; 478699; 480422; 610775; 476153; 442860; 480424; 609604; 403822; 474884; 478623; 480352; 482891; 489432; 480229; 485938; 489652;
611285; 607183; 478781; 485869; 476626; 442954; 482154; 403983; 480659; 477021; 476412; 484828; 403977; 403545; 489957; 403660; 403974; 403782; 484067;
442951; 403975; 403882; 612371; 477431; 403845; 475472; 487924; 403875; 484199; 610668; 448806; 489664; 485599; 403400; 479065; 486077; 403512; 484789;
477796; 476485; 483804; 488770; 476438; 486699; 489392; 484063; 491265; 481651; 480425
dog subset 2 (cpm)
403407; 607262; 403658; 486607; 486241; 479329; 486028; 607139; 490628; 483464; 403651; 403824; 487085; 487649; 488922; 477032; 479649; 611100; 486077;
608543; 491825; 607007; 404306; 403515; 442980; 442955; 403883; 488508; 403640; 404015; 476993; 486102; 474468; 484634; 478293; 481491; 403462; 608247;
475582; 415125; 489095; 475474; 481759; 475032; 481056; 478227; 478735; 483312; 474819; 479668; 480726; 480438; 612371; 487230; 481689; 483664; 491618;
482839; 481328; 488126; 403870; 403851; 403850; 611642; 403976; 489550; 442990; 403623; 483665; 482264; 486148; 488429; 486493; 607096; 489905; 489928;
477956; 488368; 477598; 442952; 484185; 610004; 403967; 478666; 608024; 480034; 477029; 478151; 404021; 483345; 403449; 449478; 476186; 481478; 481124;
478781; 479072; 481712; 475720; 486728; 484456; 477886; 474442; 612436; 611884; 488504; 480488; 491431; 483961; 481826; 490497; 487416; 483732; 489432;
403885; 491924; 484768; 403924; 607030; 490344; 607700; 480036; 488693; 608738; 477535; 442859; 480291; 484519; 403402; 488958; 475083; 611921; 403476;
480424; 442986; 478001; 490840; 483955; 483266; 487235; 609956; 606755; 482840; 403426; 476446; 485874; 489692; 403602; 611266; 485903; 480869; 403659;
488561; 609421; 494005; 609091; 478013; 474176; 477575; 486758; 485783; 476104; 478529; 489749; 475108; 477263; 486717; 477488; 490255; 479939; 404008;
403955; 484922; 481656; 479158; 476915; 477090; 483729; 612604; 476450; 475851; 490457; 483954; 403981; 480600; 448787; 448786; 403522; 491148; 448792;
445451; 480603; 480604; 487265; 477945; 482977; 449622; 403999; 480080; 611673; 475471; 481619; 490439; 474657; 479675; 612159; 608433; 478471; 485864;
490967; 478845; 490968; 484196; 475239; 481740; 482047; 488596; 481519; 481628; 477039; 490163; 485867; 474355; 487486; 487731; 476314; 474759; 484006;
403634; 487437; 403869; 484695; 480673; 480123; 403968; 480167; 490228; 482139; 476714; 478874; 476060; 403510; 475085; 488646; 612662; 485016; 608060;
481590; 403802; 482932; 494000; 484056; 476336; 480621; 399544; 478902; 490532; 477237; 480645; 489818; 475864; 489584; 481330; 483900; 475230; 403767;
477345; 485103; 484496; 444849; 482512; 487119; 612455; 485563; 483413; 490006; 609233; 475603; 485548; 478152; 478595; 476779; 612984; 479580; 611694;
488487; 477955; 482603; 610928; 490183; 486851; 474672; 478428; 490926; 442949; 403500; 489223; 476565; 475660; 488442; 483299; 483640; 607954; 474912;
486242; 487323; 476766; 480996; 483011; 480135; 488818; 608092; 483267; 485043; 485031; 485000; 480818; 479921; 480134; 487443; 612823; 475317; 477869;
608156; 612825; 607207; 607310; 490901; 484902; 481788; 481295; 475764; 484948; 484955; 489949; 607929; 475859; 474427; 480322; 484133; 606873; 608555;
483310; 483784; 479409; 611955; 484427; 481266; 612219; 492016; 607947; 612153; 475126; 477581; 484623; 610496; 481110; 609940; 478664; 476136; 479760;
490757; 483716; 475628; 485936; 482666; 482621; 608333; 478097; 490648; 490975; 612102; 607587; 489887; 491076; 474392; 479642; 611876; 487212; 609732;
481022; 608593
dog subset 3 (cpm)
477699; 480425; 481651; 480669; 491292; 475536; 491130; 489421; 484063; 489828; 606765; 475923; 403580; 608134; 484526; 478757; 477236; 478321; 488796;
403817; 490385; 403910; 442979; 486344; 485064; 403551; 475638; 403828; 403168; 611198; 403783; 403835; 474841; 490878; 449021; 403550; 478550; 477323;
403736; 481685; 403656; 607616; 403913; 476322; 403435; 606756; 476094; 476833; 479464; 442936; 479427; 478982; 489392; 475680; 476437; 442969; 478604;
476438; 403945; 403407; 488469; 442968; 480364; 403954; 483631; 485208; 485189; 607262; 492296; 480693; 480066; 612911; 474503; 479450; 403669; 477101;
481142; 488578; 403804; 484490; 478284; 483763; 481744; 403523; 488143; 449028; 403416; 403618; 403658; 403461; 403762; 403905; 486607; 480635; 477162;
487826; 490695; 477270; 491750; 477226; 403437; 475526; 492019; 475966; 478194; 476728; 442974; 489221; 479556; 479555; 480672; 611933; 474832; 610586;
476694; 442947; 403908; 482737; 403567; 403724; 488473; 403980; 487746; 485187; 483845; 611782; 480059; 488609; 403931; 612338; 403157; 607076; 607898;
611674; 403765; 403764; 487438; 415130; 475931; 486241; 478435; 403756; 403469; 403468; 478552; 488642; 610454; 489476; 479329; 484483; 609449; 611606;
442858; 489775; 481131; 491325; 474890; 403429; 481563; 486028; 478608; 492302; 609767; 477796; 476435; 607139; 490178; 442971; 480578; 490628; 484791;
484790; 448802; 484789; 484080; 491011; 485600; 485618; 608461; 483464; 442941; 403651; 403824; 403826; 478835; 403496; 480684; 478836; 403668; 403582;
403467; 481734; 609516; 403739; 445450; 487151; 487450; 487085; 442963; 475192; 475193; 485707; 608910; 475492; 489802; 487948; 479866; 474855; 608125;
488921; 486333; 403856; 611795; 403923; 608246; 487649; 477185; 478108; 474844; 488922; 488008; 478424; 403821; 491220; 477032; 483489; 479649; 611100;
486077; 483662; 608543; 479065; 608843; 403400; 485599; 489664; 491825; 494010; 415122; 415128; 482506; 482071; 479353; 609307; 476623; 474873; 403904;
610668; 612950; 607439; 480032; 485095; 609048; 488036; 485158; 491942; 489462; 491277; 491319; 484949; 478767; 403701; 488824; 611608; 607887; 607007;
485839; 487388; 490138; 403424; 450187; 403862; 490431; 403657; 404306; 481528; 487421; 487870; 475590; 403515; 442980; 488191; 480466; 483769; 610283;
609166; 479597; 475837; 474578; 442955; 404002; 403883; 481105; 488508; 610756; 476460; 403640; 403639; 403169; 489287; 487761; 403641; 607474; 488942;
488940; 490153; 403875; 404015; 476993; 478711; 490261; 403619; 487924; 486102; 474468; 483378; 484634; 478293; 481491; 478970; 403798; 403462; 478984;
608247; 480129; 610360; 476063; 492144; 609483; 475473; 475472; 607724; 483680; 608459; 486735; 403915; 403868; 477340; 612454; 492174; 475582; 488808;
415125; 489095; 475474; 487341; 491749; 403727; 481464; 488560; 403845; 475566; 490735; 490792; 484445; 608412; 475032; 481088; 474485; 607745; 480906;
486079; 475172; 484158; 480495; 474624; 486400; 449477; 481056; 478227; 442957; 403943; 403511; 486197; 478735; 484792; 491952; 487947; 483312; 607755;
478770; 491286; 494007; 478047; 478279; 478575; 474819; 611366; 477556; 479714; 490630; 489372; 611700; 482032; 489739; 485828; 479074; 487309; 478952;
611148; 403441; 608175; 480348; 475781; 474836; 474838; 481732; 607786; 474861; 474862; 481731; 474860; 483594; 489373; 403170; 611838; 442987; 479864;
403897; 490657; 485742; 484434; 485884; 479668; 610982; 403735; 476411; 475124; 403612; 480726; 403979; 611362; 480438; 478854; 612371; 487230; 450240;
487402; 481689; 403909; 609146; 403975; 480475; 611265; 489673; 609779; 403547; 609830; 481558; 403801; 476216; 610255; 442951; 483664; 484067; 488516;
491618; 608158; 610316; 607374; 608559; 481051; 482839; 403782; 403974; 481328; 488126; 403660; 403989; 403870; 403439; 403851; 609293; 403785; 489957;
403790; 403985; 612271; 403545; 403850; 478906; 478905; 611642; 403628; 610823; 478404; 611309; 403977; 403976; 484828; 489550; 442990; 403623; 403622;
403584; 488762; 608274; 481837; 403796; 483245; 478432; 483665; 475554; 484990; 490405; 492247; 478800; 481500; 475633; 482264; 486148; 489210; 489208;
403789; 488429; 486493; 477021; 607096; 489905; 489928; 488437; 489929; 477956; 403788; 483318; 608977; 488368; 477598; 475253; 442952; 484185; 610004;
609429; 609409; 480522; 486639; 485401; 403984; 490441; 484464; 482154; 403811; 403967; 475624; 478666; 608024; 486513; 492244; 480173; 475034; 442954;
475882; 476626; 442953; 480034; 476995; 442939; 481037; 477029; 479680; 478151; 491320; 403616; 450207; 474514; 404021; 403449; 449478; 489800; 478320;
476186; 488896; 481478; 403626; 481124; 478781; 477692; 479072; 607183; 481712; 484866; 475720; 483736; 486728; 484456; 477886; 474442; 477700; 612436;
480144; 610902; 476196; 487612; 490570; 611884; 403450; 488504; 480488; 611285; 479260; 476160; 489652; 483980; 485938; 483961; 483981; 489368; 403537;
403693; 478061; 476215; 483758; 481826; 490497; 403438; 487416; 403507; 490008; 415126; 483732; 475461; 477120; 489428; 403733; 403445; 489432; 489429;
403885; 486408; 611789; 403763; 403823; 606853; 609986; 487114; 494006; 491924; 484768; 478230; 479190; 448784; 483653; 488024; 403924; 477024; 490344;
485987; 481122; 611315; 607700; 480036; 481277; 485677; 488693; 477718; 608738; 488220; 480618; 477535; 483925; 486495; 442859; 486858; 480291; 484519;
481711; 475450; 403402; 488958; 480558; 489028; 611645; 477498; 403822; 403784; 480676; 483148; 475083; 484571; 474472; 485963; 488683; 610693; 482025;
487329; 486951; 484041; 611921; 484670; 448785; 448778; 449621; 485203; 485202; 488746; 480656; 403476; 609604; 607885; 481692; 609801; 480424; 477166;
485757; 487221; 491597; 442986; 442985; 476828; 476153; 474820; 610775; 611276; 611916; 403621; 403879; 478001; 480422; 490840; 476177; 478699; 607205;
488192; 483951; 478684; 483955; 485399; 488084; 481772; 483266; 487235; 609956; 607090; 606755; 480048; 482840; 403426; 476446; 485874; 489692; 479473;
403602; 611266; 488735; 489971; 485903; 480869; 494001; 403659; 475234; 403855; 475235; 481756; 403606; 489030; 609421; 489571; 403556; 480805; 490904;
489968; 494005; 609934; 490722; 478686; 484903; 609091; 478013; 479577; 474176; 477575; 486758; 477746; 607019; 474652; 612821; 478347; 611939; 489547;
488241; 485783; 476104; 478529; 483273; 487700; 476402; 403408; 480338; 490123; 475040; 480246; 474865; 474867; 489749; 480256; 480258; 487336; 403832;
403797; 403589; 484429; 403544; 442942; 475108; 475509; 477263; 486717; 477488; 490255; 487159; 479939; 477003; 476628; 404008; 403955; 478088; 484922;
481656; 403568; 477729; 484648; 442976; 477045; 479158; 476915; 478127; 477090; 479446; 483729; 403838; 478667; 490310; 480181; 478328; 403565; 491196;
612604; 607481; 480987; 479886; 487353; 480580; 476450; 610596; 476823; 491278; 481735; 479915; 490461; 490463; 475851; 490457; 491615; 480865; 486270;
484701; 483954; 486839; 403650; 448788; 403981; 480600; 448787; 448786; 403522; 491148; 448792; 445451; 480603; 480604; 403586; 448790; 445452; 611927;
487265; 482986; 477945; 485893; 482977; 449622; 403999; 480080; 403994; 611673; 484763; 489508; 475471; 403687; 610540; 477385; 403647; 481619; 490439;
478668; 489610; 482437; 483777; 487272; 609304; 491184; 486022; 475738; 486908; 477158; 479675; 490325; 476258; 479408; 486507; 488405; 612159; 611255;
608433; 479781; 478471; 403743; 485864; 403475; 481228; 612861; 403993; 403455; 403472; 481272; 403454; 488449; 490967; 478845; 490968; 485088; 484196;
403653; 475239; 474864; 481740; 612975; 485056; 486303; 610402; 612538; 484064; 482140; 489736; 477072; 488213; 403488; 403703; 482047; 403998; 489932;
488596; 490796; 481519; 481628; 477039; 490163; 485867; 474355; 487486; 484087; 488930; 476748; 607489; 403601; 477841; 487731; 482535; 403816; 403633;
481289; 403940; 484958; 476314; 488834; 448807; 482905; 403417; 488605; 403922; 484006; 476147; 403634; 487437; 403869; 489620; 611956; 404019; 612709;
480673; 490867; 480019; 403968; 480167; 478701; 490339; 482139; 476714; 403405; 478874; 476060; 479107; 403510; 475085; 610057; 488646; 612662; 442965;
485016; 608060; 403982; 481590; 474681; 486588; 403802; 482932; 494000; 484056; 484038; 476336; 480621; 399544; 491867; 488409; 476847; 607180; 609253;
478902; 490532; 611171; 477237; 480645; 489818; 475864; 485384; 489398; 489584; 480127; 481330; 483900; 484762; 612006; 481713; 474842; 481714; 474839;
474845; 403848; 475230; 403767; 483724; 485103; 484496; 485915; 481256; 444849; 486511; 486332; 489788; 492059; 482512; 487119; 612455; 485563; 483413;
475982; 485022; 609418; 476512; 488125; 490006; 475603; 485548; 478152; 487512; 482093; 612082; 611262; 489684; 476779; 488195; 479484; 490434; 475579;
479580; 611694; 483957; 611453; 491974; 488487; 477955; 482603; 488471; 610928; 476927; 475659; 483329; 477302; 609840; 490183; 482098; 475024; 486851;
478428; 490926; 442949; 486470; 484788; 608207; 489451; 490370; 476019; 479423; 484802; 611442; 489223; 491756; 480208; 476565; 479307; 486992; 490831;
490263; 608079; 484959; 403947; 483299; 403625; 475352; 482101; 475756; 484907; 488651; 482192; 481453; 607954; 474912; 480698; 489796; 480069; 487777;
484354; 612510; 478950; 487323; 476766; 480996; 483011; 487524; 488653; 612928; 488818; 442964; 477247; 611211; 478648; 486638; 483625; 485043; 474831;
485031; 488179; 478185; 485523; 484309; 491290; 485000; 448789; 485090; 608864; 608025; 491231; 480818; 479465; 489940; 481245; 480134; 612823; 478731;
475317; 474986; 480227; 477869; 479907; 485848; 608156; 612825; 474445; 475829; 477298; 607310; 608840; 479684; 490901; 489453; 484902; 487556; 479177;
483107; 484793; 474444; 445454; 608707; 480432; 403958; 474851; 481788; 481295; 477901; 609213; 484948; 484955; 489949; 483841; 486663; 607929; 481150;
480667; 474427; 481766; 480322; 489449; 611355; 488660; 606873; 608555; 483310; 486986; 479409; 490608; 477593; 483697; 490187; 489390; 610524; 478168;
483036; 479432; 491219; 611955; 484427; 486725; 609119; 484349; 481266; 612219; 477207; 479847; 487398; 611250; 476905; 611869; 487475; 492016; 607947;
488970; 611552; 478779; 489925; 481145; 482870; 477581; 484930; 491221; 611766; 485144; 484046; 484623; 481153; 491876; 610496; 478507; 480320; 478024;
491743; 481110; 474891; 609940; 478664; 403587; 476136; 610828; 403720; 403502; 612367; 483657; 607575; 612748; 611205; 480822; 485775; 483716; 482666;
610101; 484401; 608333; 608821; 481768; 478097; 480847; 489511; 490975; 607587; 480470; 487620; 609101; 479460; 477767; 489495; 612980; 477199; 612656;
611109; 477112; 490113; 489887; 491076; 476287; 474392; 607671; 474582; 403810; 480286; 490576; 489393; 611876; 487212; 611852; 609732; 481022; 608593;
607571; 607880; 607220; 486310; 480630; 448787
rat subset 1 (cpm)
287456; 313786; 361567; 300340; 497942; 300649; 192225; 83836; 292041; 302247; 171154; 314961; 289521; 304962; 302248; 207120; 171070; 362091; 65138; 192152; 362248; 25511; 246118; 305353; 314456;
29455; 79252; 83504; 300920; 364420; 367391; 292645; 303313; 266689; 266770; 25434; 501661; 80848; 301265; 294250; 362558; 314245; 290805; 83785; 289337; 83580; 59086; 24818; 24811; 25559; 25555;
300095; 25693; 289734; 25651; 29259; 29397; 497775; 84491; 24967; 85420; 56782; 289219; 312086; 296851; 84024; 85253; 25619; 25692; 288937; 291085; 24648; 85496; 25267; 65051; 60325; 63843; 24599;
301299; 290947; 64300; 498638; 25335; 60331; 29310; 292083; 170577; 25008; 29469; 25473; 140433; 309368; 292855; 29747; 292892; 171333; 84405; 116558; 25205; 25084; 60582; 24494; 24493; 25151;
25718; 25464; 29595; 313641; 24986; 64474; 25661; 54250; 24366; 365183; 25598; 25043; 306792; 24310; 25699; 79129; 171056; 50654; 25425; 64529; 24271; 25654; 361821; 117029; 309361; 25406; 308501;
25752; 170929; 25728; 25383; 81638; 308100; 499761; 497803; 24795
rat subset 2 (cpm)
54226; 408242; 25388; 25245; 64625; 114496; 29884; 25073; 25599; 66019; 116563; 25627; 25707; 29393; 84352; 116596; 315707; 114122; 84353; 24291; 117184;
64529; 290257; 66021; 25312; 25433; 24329; 24331; 299606; 313052; 24337; 59323; 304929; 24946; 29243; 50646; 315191; 282583; 83727; 83728; 295279; 114091;
54250; 25022; 25114; 24367; 361810; 294515; 25150; 25236; 25686; 81666; 291132; 81504; 296654; 24464; 25617; 299331; 313641; 24473; 24483; 24484; 84351; 25663;
25466; 25704; 140924; 24500; 64546; 171334; 116553; 252963; 24506; 54259; 296953; 293624; 25467; 360606; 311144; 315346; 64350; 308995; 25021; 24511; 309684;
311061; 25713; 84598; 24514; 25326; 288001; 85245; 117036; 313050; 300438; 83781; 245955; 60584; 81680; 116666; 24914; 299858; 116565; 361795; 59107; 297604;
78958; 81515; 25211; 294446; 29409; 25595; 299996; 25635; 81517; 25684; 313662; 309168; 25335; 81687; 81521; 24566; 24577; 304447; 81523; 114553; 364018;
308690; 25744; 81736; 25493; 81525; 316241; 310738; 25494; 81526; 289747; 24617; 24618; 65051; 114209; 246328; 304690; 298947; 29741; 24650; 25692; 25619;
25738; 29337; 85253; 287527; 296371; 24664; 84400; 56782; 170538; 85421; 85420; 360748; 116590; 50689; 24686; 25268; 81750; 25052; 314615; 291983; 25614;
24697; 116689; 25622; 24699; 25270; 64633; 363875; 361381; 298012; 25676; 24708; 25056; 309165; 117042; 29287; 81778; 295213; 498209; 287562; 25542; 116637;
81780; 287561; 29397; 24770; 89808; 25615; 83841; 24772; 25544; 29259; 310552; 362513; 85385; 24784; 291464; 24791; 266602; 25353; 83805; 25125; 367264; 24918;
362896; 25155; 24806; 313121; 25217; 24826; 81809; 116487; 29591; 81810; 29610; 56083; 83580; 304109; 362533; 313494; 316099; 25625; 497808; 24842; 311786;
295230; 25360; 24856; 500363; 305311; 298706; 301442; 25575; 25128; 314981; 83586; 81817; 25156; 296603; 116643; 83785; 114111; 24874; 54319; 25696; 29169;
116669; 363247; 24884; 56011; 29753; 56010; 25578; 362558; 117022; 60628; 286926; 80848; 305918; 54266; 81510; 292734; 60664; 364786; 309295; 140934; 296257;
362170; 252971; 58945; 290794; 29376; 499619; 360725; 293104; 117271; 366518; 266610; 289185; 81527; 246331; 84607; 29576; 113894; 83477; 361765; 360526;
360640; 89829; 60323; 299864; 310132; 298914; 116500; 59114; 116457; 79252; 301246; 360801; 116673; 315423; 192363; 171407; 64535; 501506; 497768; 297508;
316130; 83427; 295378; 313702; 295686; 64185; 498335; 297406; 501203; 29428; 303634; 83823; 361857; 301226; 362859; 312444; 315461; 114562; 155151; 363140;
84473; 314879; 302248; 361196; 294864; 293455; 116724; 360658; 293721; 367072; 314612; 292658; 300066; 500564; 81511; 366952; 170912; 288378; 297433; 288985;
155140; 295490; 360734; 311926; 304294; 64531; 171444; 290753; 79429; 282835; 314721; 298646; 294679; 362738; 309106; 360627; 291279; 291369; 308572; 85251;
304314; 84686; 114481; 307865; 287949; 367455; 498854; 499069; 300289; 315388; 287726; 316758
rat subset 3 (cpm)
24153; 24795; 497803; 79248; 311860; 65029; 60581; 25014; 308100; 24163; 24161; 29437; 81822; 303742; 63836; 79558; 24165; 29650; 24170; 25026; 25591; 24176;
25645; 29663; 29466; 24177; 290923; 58968; 81722; 25582; 24179; 81638; 24182; 29427; 24185; 25233; 24186; 79559; 29624; 64621; 25377; 89807; 89805; 81641;
25380; 56611; 79124; 25673; 29722; 29339; 60371; 25649; 54225; 25292; 292697; 25239; 25728; 287774; 54226; 408242; 246097; 25385; 29215; 117273; 311193;
116648; 25387; 25388; 24152; 25698; 304917; 25389; 79255; 300711; 65262; 171374; 25107; 29462; 25108; 83625; 24223; 64639; 116502; 24887; 65036; 58919; 24224;
24888; 60434; 25245; 24225; 25295; 25181; 64625; 114496; 308755; 287552; 29373; 25667; 25296; 25644; 85272; 367786; 296321; 497672; 114486; 367901; 295703;
298566; 117517; 298288; 313421; 296545; 313495; 25398; 25678; 117059; 64202; 24245; 316010; 29144; 25166; 64314; 25402; 114555; 64044; 25404; 367218; 50662;
29345; 114494; 64033; 288620; 25109; 24932; 54236; 24930; 60350; 365367; 500318; 25408; 56822; 497809; 361584; 305081; 25073; 117106; 25668; 171369; 84349;
25406; 29364; 245962; 24251; 287435; 25599; 171055; 25621; 83502; 307618; 94201; 362110; 114851; 83571; 66019; 24252; 24253; 25423; 24255; 140583; 24258;
309361; 24265; 116563; 308015; 24854; 54241; 25627; 57301; 60463; 117027; 117029; 308163; 287673; 301066; 140473; 59264; 25707; 313173; 29393; 84352; 25412;
84032; 290905; 306628; 85490; 85250; 294337; 361821; 367313; 301012; 294279; 499431; 294027; 24267; 58954; 366505; 116596; 24269; 296959; 54242; 113936;
304407; 304024; 497663; 406169; 54245; 287942; 25419; 29331; 81649; 78965; 116630; 25610; 315707; 116549; 307641; 114122; 25307; 366219; 288067; 25308; 29201;
64032; 287478; 84353; 311163; 24291; 117184; 64529; 171293; 290257; 25425; 29175; 50654; 171056; 79129; 66021; 24297; 246070; 25086; 50549; 353498; 24294;
79128; 64036; 114489; 140926; 29139; 24310; 501537; 29467; 304859; 54249; 24312; 25252; 64053; 58948; 497670; 114123; 25312; 140694; 25751; 25253; 24318;
25432; 25195; 29515; 25433; 399489; 94204; 315219; 29733; 24323; 24326; 50672; 94268; 84358; 25313; 24329; 24330; 366492; 117045; 116636; 287986; 24331;
299606; 291091; 363854; 60328; 60465; 24333; 60334; 170915; 29452; 313052; 24335; 24337; 29496; 59323; 498013; 292747; 25149; 293701; 24356; 304063; 360635;
303828; 29251; 25439; 116677; 25584; 304929; 24946; 29243; 290757; 60327; 289055; 24360; 25598; 50646; 315191; 282583; 83727; 83728; 25047; 171075; 295279;
498277; 304966; 114091; 83791; 361338; 84490; 317322; 361597; 361969; 24366; 25317; 54250; 116499; 60662; 170700; 29348; 29349; 25444; 25443; 84488; 84489;
25022; 25114; 24367; 79113; 360457; 54251; 497666; 64507; 25661; 25318; 64511; 314322; 308411; 292409; 25319; 60670; 25150; 24377; 25019; 288259; 58936; 25172;
54254; 24384; 64043; 24387; 297417; 29319; 25236; 25686; 81664; 81666; 60429; 60449; 501069; 291132; 301086; 84475; 288181; 81504; 498416; 497769; 24408;
24413; 81503; 171551; 29468; 84027; 296654; 29438; 25260; 353256; 171528; 500987; 282821; 25694; 266805; 25734; 29424; 297893; 155012; 29199; 289057; 24446;
79237; 29560; 25059; 24974; 414789; 294274; 365542; 24986; 309621; 294269; 294270; 294228; 309600; 24973; 25709; 499092; 25460; 24451; 79239; 79240; 25098;
25099; 25100; 25735; 24464; 58917; 502373; 171016; 60421; 294254; 25617; 24471; 161476; 299331; 497838; 313641; 24473; 25075; 25187; 24476; 24477; 25464;
360647; 292060; 25261; 25585; 298205; 288264; 24481; 25712; 116465; 24482; 25718; 24483; 25151; 25662; 24484; 360622; 25285; 25641; 289560; 302822; 84351;
24493; 24494; 25663; 25466; 60582; 116562; 25704; 140924; 246144; 287287; 25084; 24497; 24498; 24499; 25205; 54258; 29385; 24500; 25325; 117539; 304091;
360303; 245983; 84405; 64546; 171333; 171334; 116553; 252963; 171060; 25670; 116996; 500590; 301289; 29197; 29200; 245920; 24506; 54259; 64194; 24954; 60663;
363520; 114517; 24508; 290749; 296953; 293624; 25467; 25118; 170921; 360606; 311144; 315346; 64350; 308995; 25021; 296456; 499271; 24511; 309684; 25724;
311061; 25713; 362800; 84598; 24514; 25326; 24516; 24518; 81679; 25472; 83535; 83731; 54263; 65206; 64030; 292855; 25048; 288001; 85245; 50687; 309368; 309816;
140433; 298941; 25473; 305078; 117036; 25328; 192362; 24944; 24530; 313050; 170496; 300438; 25608; 24536; 171134; 83781; 60584; 81680; 65172; 24538; 116666;
171105; 24914; 24539; 299858; 89787; 312781; 293649; 116565; 25008; 361795; 290623; 59107; 83838; 297604; 78958; 81515; 25211; 312689; 294446; 29357; 25631;
50554; 367100; 81516; 29409; 29253; 25595; 170577; 24548; 292083; 282839; 29310; 25635; 25726; 78967; 60430; 29333; 81517; 314856; 116667; 365057; 29417;
25684; 313662; 60427; 85483; 81683; 246759; 291234; 309168; 25672; 24590; 300339; 81686; 171045; 25335; 63849; 81687; 117061; 25481; 117033; 171052; 81707;
304608; 303413; 291327; 64046; 81521; 24566; 362657; 81522; 303887; 24577; 301059; 81523; 362291; 314910; 85482; 114553; 364018; 500904; 300955; 308690;
291044; 24592; 25744; 307231; 366998; 81736; 309452; 25493; 81525; 316241; 361794; 25336; 310738; 25494; 191575; 294503; 314969; 24599; 24600; 25496; 29492;
81526; 64563; 366274; 83717; 25341; 24613; 497800; 25601; 289747; 25342; 113995; 29665; 25265; 29597; 117264; 497733; 83572; 24617; 60325; 29431; 24618;
313262; 64013; 65051; 25737; 25121; 24627; 25266; 497724; 116551; 85496; 54410; 287526; 94203; 25154; 114209; 24648; 246328; 116589; 291085; 288937; 361241;
266775; 116459; 304690; 361632; 170911; 24649; 298947; 29741; 24650; 29692; 24653; 25692; 25619; 25738; 29337; 25097; 85253; 287527; 25629; 25515; 296371;
29240; 24664; 84024; 296851; 312086; 25682; 25664; 50669; 56782; 78975; 25636; 360774; 24680; 25023; 170538; 29340; 81749; 84006; 29355; 85421; 85420; 25522;
360748; 116590; 50689; 50658; 29513; 170851; 58960; 303200; 24683; 24686; 25268; 81750; 25052; 24718; 288291; 29245; 314615; 29192; 29670; 29672; 94198; 29425;
85492; 24968; 24967; 291983; 29630; 29614; 58949; 50557; 81752; 84023; 292661; 497767; 29527; 25614; 24924; 24697; 116689; 25622; 24699; 307498; 25270; 25739;
290868; 64633; 363875; 310034; 361381; 298012; 499870; 297561; 24703; 84509; 24706; 25676; 24708; 81758; 25056; 315655; 309165; 24715; 288003; 498496; 359726;
81762; 300807; 312937; 287524; 117042; 28298; 499622; 301438; 81771; 83840; 29287; 25348; 365320; 25271; 83576; 116547; 94195; 81778; 295213; 25742; 302642;
288663; 361015; 498209; 246074; 25122; 287562; 25542; 116637; 81780; 287561; 29397; 24770; 117518; 29538; 117551; 498172; 60665; 305236; 89808; 25216; 25615;
24771; 83841; 24772; 25544; 29259; 25651; 315996; 310552; 361814; 362264; 24773; 192155; 50683; 29517; 362513; 85385; 25197; 24778; 50567; 83511; 24898; 25553;
25648; 29715; 24784; 25549; 84386; 25012; 81782; 24787; 24790; 367846; 24791; 366126; 266602; 24796; 25353; 305614; 83805; 78968; 300095; 501099; 24797; 25033;
54305; 497823; 25555; 25354; 114093; 25124; 25125; 367264; 24918; 362896; 314621; 25155; 292301; 24806; 57027; 313121; 24811; 24812; 25217; 363521; 24816;
24817; 25640; 24818; 84492; 89804; 297758; 361403; 306862; 25563; 29436; 64678; 24826; 59086; 84574; 81809; 25717; 116487; 29591; 81810; 29610; 56083; 83580;
292406; 29220; 64517; 81811; 81812; 361655; 304109; 89806; 116510; 29543; 25358; 25628; 313785; 362533; 310553; 29260; 289337; 316099; 24835; 25625; 497808;
24842; 362675; 24848; 362862; 311786; 362788; 25360; 24856; 498736; 89814; 305311; 116484; 298706; 308800; 301442; 25575; 83472; 25128; 314981; 58851; 83586;
81817; 25600; 25156; 296603; 25361; 116643; 83529; 24873; 83785; 114111; 24874; 54319; 117064; 25696; 29169; 116669; 317371; 117538; 308937; 114850; 24883;
289754; 363247; 24884; 24919; 56011; 29753; 56010; 25578; 301348; 79426; 361627; 353227; 362558; 295238; 117022; 60628; 363142; 29737; 294250; 94342; 114612;
361796; 301265; 286926; 80848; 25445; 54266; 81510; 84584; 501661; 360426; 292734; 266770; 288719; 288617; 315350; 60664; 364786; 309295; 140934; 296257;
83807; 362170; 94193; 171137; 117516; 65137; 246334; 252971; 290794; 29376; 499619; 311341; 84497; 314649; 246756; 89842; 293104; 306886; 287437; 246775;
57025; 361170; 366518; 266610; 498206; 289185; 171106; 301365; 60464; 81527; 246331; 84607; 117279; 29576; 498545; 362719; 170897; 113894; 295052; 306332;
83477; 300054; 294568; 361765; 360640; 89829; 309527; 316019; 65129; 54190; 60323; 305000; 282837; 29606; 498609; 310132; 363466; 58857; 64193; 312897;
314384; 116691; 29415; 84589; 59114; 246253; 313477; 300057; 29702; 288905; 66015; 305253; 287899; 79252; 301246; 59103; 246770; 94167; 85264; 408210; 308016;
363984; 116673; 315423; 171407; 65035; 60351; 64535; 29414; 170588; 116721; 25560; 58852; 64665; 316130; 294961; 311862; 305353; 299609; 306330; 500929;
246118; 296554; 83427; 363014; 192260; 315139; 295686; 64185; 363113; 29158; 295357; 362248; 498335; 297406; 299809; 117514; 361945; 29428; 83726; 303634;
113886; 83823; 361857; 60336; 500228; 282832; 314850; 362505; 64537; 362782; 50993; 29180; 361798; 301226; 362859; 312916; 170819; 315461; 300475; 114562;
155151; 207120; 297699; 363140; 314870; 499751; 314879; 286904; 361813; 302248; 360557; 24934; 304962; 294864; 293455; 116724; 360658; 171396; 367072; 84551;
300266; 365395; 360355; 297989; 29392; 65033; 313834; 363058; 287479; 314612; 292658; 362437; 500054; 292848; 300066; 500564; 362247; 302898; 298579; 83835;
361057; 311783; 50566; 81511; 307940; 246216; 54348; 282817; 299827; 361162; 170912; 304670; 288378; 360564; 308564; 85431; 292279; 297433; 500836; 317382;
288985; 161452; 299117; 293469; 317468; 302705; 155140; 309651; 295490; 360734; 59301; 311926; 60399; 84426; 361677; 311332; 292569; 292873; 296179; 171444;
79429; 308589; 282835; 298646; 288469; 361495; 309650; 294679; 302668; 282826; 192206; 360627; 24332; 291279; 291369; 303614; 56266; 311865; 66018; 309512;
85251; 170845; 289582; 314459; 305355; 311567; 302783; 289311; 502531; 192205; 304314; 84686; 498997; 114481; 246117; 361749; 287949; 497816; 287362; 140638;
367455; 498854; 499069; 363328; 300289; 315388; 170671; 287726; 365368; 294251; 316758; 360570; 25542
mouse subset 1 (cpm)
66172; 75736; 234911; 66102; 54698; 104871; 80707; 68045; 217847; 13196; 81702; 11426; 226641; 18074; 229228; 24000; 279028; 56436; 22134; 19124; 18618; 22152; 21899; 104859; 23886; 11504; 16591;
17082; 12738; 21933; 17167; 232889; 380713; 170716; 223920; 71790; 13602; 14070; 65969; 27226; 53761; 16975; 109929; 22427; 22339; 53791; 21825; 21824; 21803; 21454; 21354; 20927; 20608; 20788;
110877; 29856; 20344; 20343; 20292; 19271; 104009; 16912; 18761; 19073; 19044; 269823; 330260; 18979; 18815; 18792; 18791; 20725; 20719; 20704; 18613; 18605; 18595; 268591; 18788; 57385; 18126;
210009; 108156; 20288; 17393; 110616; 17201; 17199; 17169; 16992; 14725; 16803; 16779; 16776; 16773; 18050; 16542; 16533; 16407; 104099; 54131; 16161; 16159; 16198; 16195; 16190; 16181; 16176;
16175; 16004; 16001; 15894; 15558; 110611; 14268; 14173; 110135; 14079; 14069; 13717; 13610; 11421; 13166; 13057; 13051; 13040; 13036; 13030; 76703; 12814; 12834; 12774;
12675; 12505; 12483; 12511; 12047; 11816; 11810; 11808; 11608; 224530; 100705; 11303; 20716
mouse subset 2 (cpm)
11820; 16617; 216869; 12062; 12122; 12124; 51800; 20778; 16149; 12591; 11773; 17228; 12803; 12842; 12843; 26410; 12988; 13003; 12387; 66473; 109660; 13030;
13035; 13058; 13406; 15200; 13649; 109901; 50701; 269587; 13866; 13869; 13982; 14067; 14071; 14058; 19229; 14114; 14115; 14118; 14119; 14129; 14134; 14173;
14182; 14183; 14186; 99571; 14229; 56484; 14360; 14734; 14677; 14682; 14756; 14784; 227753; 15439; 14828; 15519; 15550; 21923; 16002; 16009; 16150; 16177;
16180; 16184; 16186; 16199; 16160; 16162; 16163; 16164; 16334; 16331; 27056; 54123; 16367; 16400; 16401; 16402; 381924; 16408; 16409; 16412; 16414; 16420;
16421; 16451; 16452; 16453; 16623; 16644; 16647; 226519; 432548; 16818; 16835; 16854; 19039; 16878; 16880; 70361; 16948; 16971; 14725; 16976; 16994; 268977;
17000; 57278; 17096; 17105; 17118; 17136; 17756; 17181; 17202; 17242; 17287; 17288; 17150; 26403; 17393; 17395; 17698; 15235; 17869; 17904; 17926; 17969; 17970;
17984; 18016; 18033; 18035; 18036; 18037; 18049; 18073; 18133; 18413; 18787; 18489; 268591; 16922; 241196; 30955; 18708; 18709; 18763; 18791; 18792; 18793;
18803; 234779; 18815; 18816; 18830; 18968; 18976; 116847; 19073; 18753; 18760; 18761; 19090; 26413; 26417; 19122; 19123; 19128; 547443; 19152; 19171; 14083;
19246; 15170; 19247; 19264; 19299; 271457; 19353; 19358; 19359; 218397; 19645; 19659; 19697; 627889; 20085; 20194; 20196; 394252; 20248; 20293; 20302; 20303;
20304; 20306; 20292; 20296; 20312; 15529; 53378; 20315; 20339; 20343; 20345; 20319; 230126; 20416; 105243; 67804; 20650; 20692; 20730; 20750; 20779; 20848;
20849; 20850; 20852; 20963; 21333; 26409; 21356; 21819; 21808; 21810; 21812; 21813; 21814; 21817; 21824; 21825; 21844; 21885; 21894; 21922; 21929; 21937; 21938;
22059; 621054; 22030; 12462; 22095; 22139; 22147; 22165; 54721; 546265; 22218; 22287; 22242; 12818; 22278; 22282; 22324; 22325; 269523; 22339; 22341; 22346;
22350; 22359; 22370; 22371; 22596; 22612; 54401; 22627; 22628; 22631; 16975; 16178; 12767; 21789; 65969; 21821; 17123; 12587; 108075; 18710; 241226; 16151;
230233; 13011; 11800; 12703; 56455; 21951; 384783; 54709; 69150; 13626; 12123; 230979; 14082; 433375; 18187; 18186; 216233; 22403; 18412; 12042; 12234; 18452;
53886; 53859; 12702; 16801; 55960; 18414; 14924; 16413; 67474; 26941; 19099; 11504; 67384; 20016; 20602; 20970; 74360; 67298; 266781; 74383; 14181; 56805;
21353; 59027; 50498; 66905; 14694; 57257; 23897; 71890; 22193; 12331; 30926; 55985; 12468; 67268; 14007; 14674; 67903; 103213; 11811; 13660; 216197; 76614;
12539; 12721; 56289; 53975; 73192; 18074; 56541; 12465; 74105; 260302; 14376; 69632; 216148; 101540; 66797; 106039; 100017; 237038; 16797; 170770; 94230;
69865; 69165; 319655; 72999; 83430; 59308; 67838; 94217; 108086; 24071; 107975; 67620; 76829; 54635; 78903; 11610; 68558; 72183; 24058; 14230; 12032; 64113;
13418; 71960; 12822; 108723; 217124; 74330; 170571; 224024; 246154; 76905; 117158; 218624; 446099; 209590; 71753; 66599; 16772; 225471
mouse subset 3 (cpm)
232345; 20716; 11303; 11305; 11350; 76507; 107476; 110446; 224530; 11423; 11434; 11431; 11459; 11461; 71885; 60595; 11477; 11486; 11487; 11518; 11535; 11545;
11555; 11556; 11764; 14797; 11576; 11593; 11595; 11596; 11604; 11606; 11608; 11609; 11629; 11651; 11652; 11657; 11658; 11690; 11648; 11699; 11727; 11600; 11601;
16790; 16952; 12306; 11746; 11747; 11785; 20219; 11797; 11799; 11806; 11807; 11808; 238055; 11812; 11813; 11815; 11816; 11818; 11820; 16617; 14102; 14103;
11847; 11848; 228359; 11859; 109689; 216869; 50518; 11898; 11905; 11910; 11911; 11920; 11946; 11947; 54140; 26361; 12000; 26362; 12010; 12015; 12018; 12028;
12038; 12443; 12043; 12048; 12050; 12062; 12064; 12096; 12111; 12122; 12124; 12144; 104184; 12156; 110075; 12159; 12161; 12162; 12169; 329547; 12189; 12229;
12258; 12259; 12266; 12273; 230558; 110382; 69379; 230099; 12286; 12305; 12308; 12317; 12323; 12796; 12330; 12362; 12366; 12367; 12363; 12370; 12389; 12393;
12406; 12428; 12444; 12466; 12480; 12504; 12507; 12525; 12475; 12478; 21940; 12519; 12524; 21941; 233274; 12490; 12491; 20778; 12492; 12494; 21939; 21947;
12505; 16423; 12506; 12508; 12514; 16149; 12518; 15985; 12520; 12550; 12562; 12567; 107951; 12575; 12576; 12591; 12606; 12608; 13032; 12638; 12649; 12652;
12675; 12715; 11773; 23844; 12759; 67300; 17228; 12768; 12772; 12771; 12774; 12458; 12775; 12776; 59289; 16995; 12803; 12804; 12842; 12843; 12824; 12825; 12826;
12827; 12831; 12832; 12833; 12834; 12835; 12836; 12815; 12817; 12821; 12846; 23849; 69928; 26410; 12870; 109697; 232680; 12873; 56373; 12876; 12740; 12892;
12914; 12915; 12928; 12929; 12944; 12950; 12960; 26416; 12977; 12981; 12985; 12988; 12995; 13000; 13001; 13003; 13010; 58214; 13014; 13019; 14219; 83429; 12387;
12388; 66473; 109660; 13030; 13033; 13035; 13036; 13038; 13040; 13051; 13057; 13058; 13077; 13095; 13106; 624900; 110115; 13072; 13132; 13136; 13138; 13163;
13179; 11421; 13193; 13198; 13211; 11537; 13361; 13383; 23859; 53310; 13385; 192166; 13406; 13429; 13430; 13482; 13489; 13491; 13492; 13498; 109620; 15200;
13555; 230857; 72962; 13609; 13614; 13617; 13618; 13636; 13637; 13645; 13649; 13653; 13836; 13684; 13685; 208643; 109901; 50701; 66222; 15568; 13728; 13043;
13805; 13806; 56205; 328572; 13819; 269587; 13856; 13866; 13867; 13869; 78943; 14113; 13982; 13983; 26379; 23871; 23872; 18612; 104156; 14061; 14062; 14063;
14066; 14067; 14069; 14071; 14058; 109821; 74145; 14060; 14080; 14079; 19229; 14114; 14104; 14115; 14118; 14119; 14125; 14127; 14128; 14129; 14131; 246256;
14134; 110196; 14151; 14156; 14735; 14159; 14161; 110135; 14164; 14173; 14175; 14176; 14177; 14178; 14179; 14180; 14165; 14168; 14182; 14184; 14183; 14186;
99571; 14191; 14198; 14205; 15229; 14254; 14257; 14264; 14268; 14272; 110606; 14281; 14282; 14284; 14293; 14319; 14345; 14360; 14381; 14375; 14450; 14456;
14460; 14463; 14473; 14537; 14580; 14583; 14598; 14603; 14734; 14677; 14678; 14682; 14683; 14695; 54214; 14723; 14733; 14756; 23832; 12766; 71223; 14784; 14783;
50915; 107746; 14810; 14815; 14825; 330122; 14827; 56637; 227753; 14871; 14886; 14939; 15270; 15116; 15117; 15118; 15162; 15194; 433759; 12628; 15216; 50702;
15234; 15242; 15251; 15277; 14972; 547349; 14964; 14998; 15002; 14960; 14968; 14969; 15040; 14963; 15007; 15288; 433238; 15366; 15368; 15369; 15370; 15375;
15376; 15377; 15378; 225307; 15439; 15458; 15461; 94175; 15469; 15499; 193740; 14828; 15507; 69253; 15519; 432551; 15550; 15551; 15560; 15565; 21923; 15874;
15891; 15894; 15896; 78369; 15900; 15901; 15903; 15974; 15975; 15977; 15978; 15979; 16000; 16001; 16002; 16004; 16008; 16009; 16010; 16011; 16012; 29817;
209268; 16150; 16175; 16176; 16177; 16180; 16181; 16183; 16184; 16185; 16186; 16188; 16189; 16190; 16191; 16193; 16194; 16195; 16196; 227288; 12765; 16199;
16153; 16154; 16155; 16156; 621887; 16159; 16160; 16161; 16162; 16163; 16164; 16165; 16168; 16170; 21942; 16171; 16173; 16323; 15945; 16334; 16331; 231070;
16337; 23920; 16179; 16403; 16362; 16363; 27056; 54123; 16367; 16398; 16399; 16400; 16401; 16402; 104099; 381924; 16408; 16409; 16410; 16411; 16412; 16414;
16416; 192897; 16419; 16420; 16421; 320910; 16451; 16452; 16453; 16476; 16478; 16480; 16523; 16514; 16531; 140493; 16534; 16542; 16590; 18050; 16623; 16621;
16644; 16647; 16691; 16728; 16773; 16774; 16775; 16776; 16777; 16779; 16780; 226519; 16783; 16782; 16784; 432548; 16816; 16818; 16819; 16835; 16846; 16847;
107753; 16854; 16878; 16880; 16885; 15450; 70361; 240028; 16948; 16956; 16971; 14725; 16974; 16973; 16976; 16992; 16994; 72054; 268977; 16998; 17000; 57278;
17096; 17105; 17113; 17118; 17126; 17127; 17128; 17130; 17131; 17136; 17161; 17756; 17169; 17194; 17196; 17199; 17200; 17201; 17202; 17203; 84004; 17210; 17221;
17242; 17246; 17258; 26401; 26408; 17283; 17287; 17288; 17295; 17150; 17311; 12265; 17319; 17329; 17342; 17345; 26403; 110784; 17380; 83995; 17390; 17392;
17393; 17394; 17395; 17384; 17385; 17381; 17386; 17387; 58223; 17523; 17533; 17535; 17698; 15235; 17769; 238505; 17829; 623609; 140474; 17869; 17874; 17926;
17932; 17937; 27354; 17969; 17970; 17972; 17973; 17984; 66108; 18003; 18015; 18016; 18018; 18022; 18033; 18034; 18035; 18036; 18037; 18038; 18045; 18049; 18073;
18102; 18119; 110109; 18125; 18126; 18127; 18128; 18129; 18133; 54631; 18216; 18431; 27047; 18383; 18386; 18387; 18390; 18413; 18430; 94045; 18439; 18441;
18442; 233571; 18550; 18472; 18787; 18788; 18479; 18489; 18491; 67466; 268591; 18538; 18549; 238871; 18590; 18596; 228026; 18605; 209558; 18613; 20317; 18654;
18667; 18671; 16922; 20704; 20724; 20719; 20725; 20723; 241197; 20713; 241196; 18704; 18706; 18712; 30955; 18708; 18709; 628161; 18763; 18783; 18791; 18792;
18793; 18803; 234779; 18806; 18815; 18816; 103968; 18817; 18830; 18968; 18970; 18976; 18979; 330260; 269823; 19015; 19016; 18646; 19073; 108079; 18747; 19085;
18750; 18751; 18753; 18754; 18755; 18759; 320795; 18760; 18761; 18762; 19090; 26413; 26417; 26419; 19094; 26420; 26415; 26395; 26396; 26397; 19109; 19122;
19123; 19128; 547443; 19699; 19146; 19144; 19152; 19164; 19167; 26442; 19170; 19173; 19177; 16913; 16912; 19171; 19186; 19191; 19204; 19211; 19217; 19219;
19222; 19224; 19225; 14083; 19242; 19246; 15170; 19247; 19264; 19290; 19299; 110078; 97541; 271457; 19353; 19356; 19358; 19359; 19361; 19365; 110157; 19384;
218772; 218397; 19645; 19651; 19659; 19684; 19697; 19702; 106344; 93726; 24014; 19877; 19883; 19888; 68275; 627889; 19951; 624713; 627640; 20111; 20085;
629957; 20133; 20181; 20182; 20661; 20201; 20202; 20194; 20196; 20203; 20229; 20239; 246103; 394252; 20248; 20249; 20276; 20293; 20302; 20303; 20304; 20306;
20292; 20296; 20295; 20297; 20299; 56221; 20311; 56066; 20312; 20969; 15529; 20971; 53378; 20315; 20339; 20343; 20344; 20345; 20350; 26398; 20319; 20383; 67996;
20387; 20388; 20389; 20393; 230126; 20416; 20440; 20481; 20525; 17254; 20538; 13162; 15567; 11987; 20541; 105243; 214084; 20568; 20614; 20650; 20652; 20656;
20660; 20683; 20687; 20692; 20375; 20730; 20737; 20750; 20742; 20779; 20787; 20788; 20807; 20604; 20605; 20606; 20607; 20608; 20609; 19143; 20846; 20848; 20849;
20850; 20852; 20869; 20963; 20997; 21333; 11491; 26409; 21354; 21355; 21356; 66826; 21390; 21405; 21410; 21454; 21682; 21687; 21749; 21750; 22041; 21418; 21786;
21788; 22042; 21819; 21803; 21804; 21808; 21809; 21810; 21812; 21813; 21814; 21817; 21824; 21825; 21826; 21828; 50492; 21832; 21833; 21843; 21844; 21846; 21857;
21858; 21859; 21869; 15898; 21885; 21897; 24088; 142980; 21898; 53791; 21922; 21926; 21929; 21930; 21937; 21938; 81877; 22059; 22062; 21990; 22027; 22029;
22030; 22031; 622602; 22095; 22139; 22151; 22164; 22165; 22166; 54721; 22173; 546265; 22218; 22287; 22235; 22242; 12818; 22260; 22278; 22282; 22288; 22324;
22325; 22329; 269523; 22333; 22337; 22339; 22340; 22341; 22346; 22350; 22353; 22359; 22370; 22371; 22376; 215280; 22390; 22421; 22431; 22433; 22596; 22612;
22632; 54401; 22627; 22628; 22631; 22637; 22695; 20841; 235320; 16975; 65111; 16178; 12767; 20347; 16497; 53761; 224727; 434585; 53817; 193742; 27226; 21789;
65969; 14283; 17123; 12587; 17979; 14070; 53357; 108075; 223920; 22022; 22021; 16370; 18710; 241226; 16151; 230233; 13011; 56449; 11800; 228355; 21943; 56505;
22061; 108112; 12703; 21951; 384783; 54709; 211429; 13350; 13611; 71609; 56453; 13626; 19766; 69583; 22035; 11490; 11502; 230979; 14082; 21934; 433375; 107527;
16182; 80903; 18187; 18186; 216233; 12633; 22403; 21807; 211914; 20698; 18412; 12427; 11767; 12042; 14106; 83767; 12234; 53886; 53859; 12702; 12642; 54199;
12737; 54189; 16801; 26909; 58249; 18554; 53978; 17082; 18414; 194590; 30960; 14924; 30939; 17681; 73086; 17164; 19157; 14739; 56382; 26941; 11450; 230597;
79456; 13666; 50931; 240913; 545532; 216725; 11504; 67384; 20016; 64292; 57080; 11758; 11307; 56640; 229933; 56489; 20970; 74360; 67298; 266781; 329504; 12355;
20186; 14181; 23797; 170789; 11768; 27416; 20928; 22259; 50498; 14251; 66905; 72413; 51796; 239273; 23928; 21899; 27403; 16598; 22143; 22152; 227613; 14694;
22601; 52858; 66513; 23897; 22193; 67252; 12331; 56403; 23876; 30926; 68576; 19124; 55985; 12468; 12461; 56338; 50706; 14007; 80901; 13018; 14674; 16562; 67903;
103213; 59046; 17768; 12769; 327826; 20301; 15442; 13116; 19325; 24111; 110147; 11811; 216197; 26754; 93672; 30806; 12539; 12721; 229228; 20901; 56289; 73914;
227619; 73192; 106648; 106504; 18074; 195046; 27007; 226641; 12465; 74105; 260302; 240725; 69632; 50934; 12419; 56708; 65086; 230073; 23821; 17087; 100226;
75292; 54725; 74637; 216148; 101540; 101185; 626834; 106039; 100017; 56407; 66409; 18782; 74277; 18130; 54450; 23892; 237038; 16797; 244667; 28000; 53416;
66824; 56480; 75029; 69865; 212999; 69165; 76281; 69511; 50490; 224480; 319655; 116849; 20371; 72999; 16842; 18080; 67869; 170743; 170744; 270669; 83430;
68816; 59308; 67838; 58991; 94217; 94226; 99439; 81897; 22417; 54473; 54485; 108857; 72667; 317652; 241639; 107975; 54635; 56632; 78903; 11610; 75788; 57775;
240058; 68558; 623773; 70008; 68626; 50501; 14230; 13706; 64113; 13418; 66313; 64385; 96979; 71785; 74493; 12822; 83431; 66899; 93835; 16396; 624153; 27217;
14841; 229317; 246691; 108723; 217124; 72780; 74330; 193813; 77125; 224024; 112407; 216799; 246154; 66113; 76905; 117158; 218624; 446099; 106759; 209590;
71753; 227638; 66599; 246779; 16772; 77697; 360013; 20302
dog subset 1 (dm)
485189; 487265; 488340; 403822; 474589
dog subset 2 (dm)
480578; 484790; 484789; 487649; 489593; 489464; 487341; 403718; 478905; 480291; 490921; 487329; 487221; 403987; 403779; 403955; 480686; 609901; 482007; 476423; 483299; 481627; 612868; 476135;
488173; 611038; 485546; 480379; 485811; 610361; 491947; 485154; 479482; 483643; 487346; 484055; 485365; 483127
dog subset 3 (dm)
484526; 478321; 493972; 403910; 483701; 480605; 442936; 490679; 491939; 607262; 474503; 403416; 607482; 442974; 403980; 480578; 484790; 484789; 485600; 485618; 487151; 487649; 485599; 494010;
415128; 489593; 489464; 404306; 403641; 488940; 487341; 490792; 489143; 489141; 403718; 484024; 403735; 478854; 403801; 442951; 403782; 403974; 403660; 403785; 403985; 478905; 403628; 403584;
403693; 431695; 478230; 442859; 480291; 477498; 403784; 490921; 487329; 487221; 479577; 477575; 486758; 477746; 607019; 403589; 484429; 442942; 403987; 403779; 403955; 480686; 483729; 403586;
486022; 609901; 485864; 488449; 488213; 477841; 403816; 403922; 403869; 482007; 403802; 476423; 483299; 490812; 483760; 481627; 478054; 609213; 488173; 491947; 483643; 488830
rat subset 1 (dm)
rat subset 2 (dm)
54241; 60463; 117029; 315707; 83721; 79113; 81668; 29385; 25493; 60355; 24613; 24627; 24695; 56813; 363875; 29622; 192215; 25570; 116692; 59114; 83633; 59315; 282840; 116647; 171039; 192253; 65034;
246212; 311532; 25501; 171297; 361604; 117096; 80977; 297872; 499017; 24805; 60564; 60565
rat subset 3 (dm)
63836; 29650; 24925; 24176; 25238; 140670; 81638; 79121; 25388; 79255; 24224; 25404; 54241; 60463; 117029; 301066; 140473; 58954; 315707; 171056; 24297; 25086; 83721; 24329; 360635; 116677; 79113;
314322; 292409; 29705; 29709; 81666; 81668; 24414; 502373; 497838; 25712; 25718; 24493; 24494; 60582; 287287; 24498; 29385; 25325; 25670; 314856; 291234; 26955; 362657; 81736; 25493; 24600; 60355;
24613; 24627; 25522; 116590; 50689; 84023; 292661; 29527; 24695; 56813; 363875; 29622; 309165; 117518; 117551; 25648; 192215; 83805; 25124; 306862; 361655; 116510; 24835; 24842; 25570; 83785;
116692; 59114; 497738; 83633; 307479; 170819; 282840; 116647; 171297; 80977; 305434
mouse subset 1 (dm)
109689; 20312; 54393; 18126; 18189
mouse subset 2 (dm)
67300; 12772; 12774; 12988; 13542; 13543; 14191; 14715; 12765; 18035; 18195; 18386; 238871; 19227; 19228; 19353; 19730; 277973; 22045; 19158; 26941; 242425; 104079; 18232; 107503; 192167; 19894;
244281; 68070; 11426; 217692; 228765; 18231; 245537; 83671; 216856; 80976; 269589; 83672; 20980; 60510; 54524
mouse subset 3 (dm)
60595; 11487; 11554; 11555; 110355; 71770; 11608; 11799; 11845; 170735; 216869; 11911; 12043; 110279; 12273; 12389; 67300; 12772; 12774; 12776; 59289; 23849;
12988; 13051; 13077; 13106; 13542; 13543; 13649; 18612; 14063; 14191; 14281; 14293; 14394; 14406; 14682; 14715; 14816; 15461; 432551; 15978; 16001; 16175;
16176; 16181; 16189; 16193; 12765; 16153; 16168; 17246; 17345; 626051; 17769; 18033; 18035; 18125; 18127; 18195; 18386; 238871; 18762; 26413; 26417; 26419;
19094; 19219; 19222; 19225; 19227; 19228; 19353; 19730; 19697; 20295; 20299; 11987; 277973; 20779; 20846; 21418; 21843; 21857; 21926; 22059; 22045; 22339;
19158; 26941; 18191; 18190; 242425; 76217; 93672; 107503; 192167; 245537; 80976; 76071
dog subset 1 (k)
476724; 478471; 482355; 442971; 403816; 404021; 475472; 475473; 476131; 445451; 478666; 478432; 479098; 481688; 442971; 610935; 479912; 480809; 474938; 480356; 612911; 403979; 486625; 607923;
608021; 403435; 475851; 480143; 488850; 485587; 476133; 477858; 475666; 474637; 480893; 485435; 475172; 481608; 403635; 489687; 403635; 489687; 478478; 490326; 483426
dog subset 2 (k)
403945; 484472; 479329; 403651; 403824; 487649; 488922; 486077; 607439; 607007; 404306; 442955; 488508; 475472; 475474; 475032; 474485; 483312; 486671; 403612; 491618; 476412; 403789; 403788;
609429; 609409; 444857; 478151; 403616; 450207; 483345; 611737; 478781; 610902; 479737; 476215; 609832; 489432; 403885; 477535; 442859; 488958; 480424; 477166; 484431; 482840; 476446; 403602;
611266; 475234; 489816; 609421; 485783; 483729; 403787; 480143; 475851; 491615; 403999; 478471; 485864; 481628; 477039; 403444; 487486; 487731; 403869; 479098; 475085; 608060; 484056; 476336;
480621; 477237; 480645; 489584; 608147; 481713; 403767; 485103; 483121; 477480; 490263; 483640; 481479; 478728; 489832; 609018; 477901; 479244; 606923; 479868; 403553; 480062; 609854;
611189; 608531; 475258; 607530; 487128; 482745; 490046; 479616; 610442; 479383
dog subset 3 (k)
491292; 475536; 489828; 475923; 477587; 403580; 478250; 608134; 478321; 478489; 490385; 475251; 490878; 607537; 403021; 476322; 403435; 476094; 479325;
479246; 479259; 403945; 403407; 403954; 475240; 486545; 474503; 479450; 403669; 487718; 483763; 403523; 449028; 403618; 607482; 403658; 403762; 403905;
484472; 486127; 477162; 476728; 442974; 489221; 479555; 479354; 480416; 476694; 483745; 480118; 403567; 487432; 487746; 485187; 403931; 403646; 403469;
610454; 474391; 489476; 479329; 485790; 442858; 489775; 474890; 403429; 481563; 403858; 484790; 448802; 484789; 403651; 403824; 403496; 487948; 478806;
487786; 479441; 403856; 611795; 487649; 475928; 477185; 474844; 488922; 479649; 486077; 608543; 608843; 403400; 494010; 474177; 415122; 415120; 415128;
610195; 479740; 415127; 403904; 607439; 610935; 490813; 478767; 479935; 607007; 491296; 403657; 404306; 481528; 442980; 480466; 491860; 610283; 442955;
480113; 477373; 403883; 488508; 493995; 403639; 607474; 489090; 478711; 474468; 475473; 475472; 475474; 487341; 403845; 609612; 479746; 475032; 481088;
474485; 478394; 403755; 480495; 609822; 609630; 491952; 486915; 483312; 478047; 474819; 474939; 479912; 479911; 611366; 477556; 481801; 486671; 479074;
487551; 478952; 403441; 609564; 611838; 490657; 485742; 484434; 485884; 478450; 610982; 403612; 403612; 480726; 611362; 403909; 609146; 403975; 480475;
609779; 403801; 442951; 491618; 610316; 608559; 403785; 403985; 403850; 403628; 610823; 442990; 403796; 488540; 476412; 489208; 403789; 488429; 486493;
477021; 489905; 488437; 477956; 403788; 608977; 609423; 609429; 609409; 483002; 475624; 444857; 492244; 481037; 478151; 403616; 450207; 474514; 483345;
484521; 442861; 611737; 403626; 481124; 478781; 477886; 476070; 610902; 478092; 480488; 484622; 481839; 477871; 610519; 479737; 403693; 478061; 476215;
403438; 442978; 480229; 479417; 489428; 403733; 403445; 489432; 489429; 403885; 486408; 403823; 491924; 403768; 474729; 804480; 478230; 448784; 483653;
613013; 611315; 477535; 479552; 478813; 442859; 488958; 479051; 480558; 606932; 480676; 478754; 491866; 476749; 478343; 482025; 488746; 403476; 612316;
480424; 477166; 403879; 484431; 482840; 403426; 476446; 485874; 488167; 403602; 611266; 475234; 489816; 481756; 609421; 494005; 609934; 484903; 609091;
477575; 477746; 480454; 476402; 403408; 403797; 403589; 442942; 475108; 403955; 477045; 479446; 483729; 489798; 477308; 478667; 477499; 480580; 480141;
403787; 480143; 475851; 475852; 491615; 610295; 448788; 403981; 480600; 448786; 403522; 491148; 448792; 403586; 490356; 487265; 449622; 403999; 480080;
403994; 609304; 479275; 479279; 478478; 480178; 403559; 486507; 612159; 479781; 478471; 403743; 485864; 609351; 488449; 490967; 403892; 484196; 485079;
477072; 403703; 403998; 481628; 477039; 485867; 403444; 487486; 487731; 403633; 481289; 403940; 403417; 403922; 487437; 403869; 403536; 490080; 480167;
487509; 479098; 403574; 476060; 608979; 475085; 608060; 403982; 486588; 403802; 482932; 484056; 476336; 480621; 399544; 476447; 611171; 477237; 480645;
489584; 608147; 483900; 403767; 485103; 482509; 489632; 403482; 482366; 484796; 607106; 490263; 490935; 485588; 483640; 484822; 476708; 482364; 481479;
488653; 612928; 478728; 489832; 478185; 609018; 485470; 608707; 609605; 477901; 488072; 612771; 481150; 479595; 479244; 477300; 611038; 606923; 612558;
477186; 479868; 478211; 474434; 403553; 609854; 478507; 475258; 403629; 607530; 482745; 482744; 477463; 484401; 490046; 607858; 442958; 479775; 609866;
479616; 477163; 610442; 607671; 479383; 474422; 486608; 491232; 479574
rat subset 1 (k)
rat subset 2 (k)
287774; 81613; 25599; 29393; 84352; 315707; 114122; 64529; 29467; 25312; 25433; 24329; 313052; 59323; 24366; 54250; 24367; 25150; 25019; 81504; 57298; 171016; 294254; 24484; 292892; 24516; 24518;
300250; 313050; 25608; 24536; 245955; 114903; 25631; 288532; 365057; 25335; 81687; 25744; 81736; 25494; 65051; 25737; 25344; 25692; 85253; 287527; 84024; 29441; 56782; 24686; 309165; 24615; 85247;
81778; 25742; 25544; 25353; 83805; 29591; 81810; 25085; 304109; 24842; 29545; 314981; 296603; 54319; 25696; 29169; 56011; 29753; 362558; 64158; 294250; 80848; 54266; 312492; 498183; 292645; 113894;
60323; 116457; 307309; 288564; 25514; 312916; 84398; 363544; 116724; 113906; 289144; 315097; 85311; 361769; 25309; 366196; 307126; 114246; 302963; 292071; 294846
rat subset 3 (k)
116632; 116631; 311860; 65029; 29437; 25365; 81822; 29275; 303742; 29650; 24172; 25591; 25690; 24185; 24192; 266802; 25380; 56611; 25673; 79125; 290527;
287774; 54226; 117273; 25612; 315305; 79255; 300711; 65262; 171374; 304127; 64639; 24887; 58919; 24888; 25245; 25295; 25181; 81613; 83470; 29373; 25644;
296137; 117517; 313421; 497751; 24242; 64202; 29153; 29154; 25402; 58918; 367218; 50662; 29345; 24932; 25660; 171369; 25406; 24251; 29186; 287435; 25599;
171103; 83502; 307618; 114851; 83571; 24253; 81645; 60463; 117027; 117029; 29393; 84352; 290905; 304024; 25419; 25420; 24277; 81649; 78965; 315707; 64462;
116549; 114122; 29317; 24291; 64529; 290257; 29175; 50654; 24296; 24297; 361523; 246070; 293985; 29296; 171522; 25086; 65210; 171352; 286929; 24309; 29139;
29467; 24314; 65162; 29475; 25312; 25253; 25433; 266606; 25313; 24329; 24330; 287986; 299606; 363854; 314436; 25043; 60465; 170915; 313052; 25315; 65030;
24337; 59323; 25149; 303828; 29251; 304929; 290757; 306761; 60327; 315191; 498277; 361969; 24366; 54250; 24367; 79113; 25661; 171049; 29292; 25150; 24377;
25019; 363735; 24383; 24387; 25283; 25259; 84475; 59075; 59076; 64317; 29328; 81504; 24413; 296654; 24421; 501110; 24423; 57298; 64352; 499689; 24424; 29438;
25260; 316218; 64055; 29424; 24443; 155012; 24446; 25460; 25098; 25099; 25100; 25735; 79256; 58917; 171016; 294254; 294254; 25617; 161476; 25075; 24476; 24477;
25464; 360647; 25261; 25712; 25718; 24484; 25285; 289560; 287287; 24498; 25325; 117539; 116553; 29197; 24504; 292892; 170921; 311144; 315346; 308995; 296456;
24511; 309684; 60583; 24516; 24518; 29553; 292855; 25048; 300250; 360625; 50687; 24944; 313050; 25608; 24536; 56646; 171134; 245955; 25474; 25476; 114903;
24539; 299858; 311337; 81515; 297176; 25631; 29456; 312538; 316273; 29728; 291885; 288532; 314856; 116667; 365057; 25332; 246759; 60331; 291234; 315606;
300339; 81686; 171045; 25335; 63849; 81687; 117061; 25481; 81707; 81521; 362657; 288770; 85482; 25744; 25492; 83619; 81736; 25494; 499194; 191575; 291084;
25496; 54278; 302552; 25502; 25341; 497733; 24617; 56816; 65051; 25737; 25344; 29692; 25692; 25619; 25738; 25096; 85253; 287527; 84024; 29441; 114503; 25682;
56782; 170538; 29340; 29355; 85421; 116590; 114495; 29245; 29192; 81752; 84023; 29527; 25614; 363875; 24706; 315655; 309165; 116468; 288414; 288003; 304528;
474169; 83840; 295214; 24615; 85247; 81778; 445415; 25742; 287562; 116637; 81780; 287561; 29397; 117518; 298006; 117551; 305236; 171371; 89808; 24772; 25544;
29259; 25651; 362264; 24898; 25548; 113893; 29219; 64347; 291794; 24786; 24790; 24791; 24796; 25353; 305614; 83805; 81800; 25124; 25125; 83783; 25155; 171046;
64678; 59086; 29591; 81810; 56083; 25085; 304109; 29543; 25358; 25628; 29260; 24835; 497808; 24842; 29268; 24856; 25232; 29545; 24860; 25575; 83808; 304944;
314981; 296603; 25361; 24873; 83785; 114111; 54319; 25696; 29169; 116669; 84495; 24919; 56011; 29753; 362558; 64158; 60628; 80848; 54266; 500526; 292645;
500592; 84385; 113894; 155183; 316101; 60323; 287739; 83504; 316061; 116457; 29455; 140665; 312331; 307309; 501070; 65035; 60351; 288564; 25514; 290409;
64537; 290408; 312916; 361466; 361020; 314870; 117287; 295050; 171039; 84398; 361532; 311546; 363544; 116724; 313658; 84016; 113906; 117089; 315097; 161452;
85311; 25309; 113992; 366196; 114246; 116469; 288753; 308589; 302963; 289076; 64803; 309004; 316561; 292071; 296178; 294846; 299828; 300160; 246234; 25592
mouse subset 1 (k)
57875; 20750; 93695; 12759; 21857; 16854; 110135; 14161; 17219; 20296; 16644; 12505; 15945; 22223; 326623; 12759; 18104; 14187; 14862; 100494; 14859; 14776; 11910; 15507; 217845; 67623; 15360;
20775; 12306; 20194; 20200; 20531; 14915; 52118; 72082; 28254; 207151; 102570; 140483; 18242; 18263; 12583; 19733; 239134; 14473; 17279; 13370; 13370; 20617; 30935; 210622
mouse subset 2 (k)
11818; 545936; 16149; 12842; 12843; 12988; 13003; 13030; 13198; 13406; 15200; 13649; 269587; 13869; 110135; 14173; 99571; 14360; 14375; 14784; 14864; 14917; 94175; 193740; 16009; 54131; 16399;
16416; 16476; 16478; 16678; 16818; 16846; 16847; 19039; 233187; 14725; 17127; 17220; 26408; 17393; 17395; 18016; 18033; 18073; 268591; 18538; 18673; 18791; 18793; 18815; 18816; 18979; 18984;
19073; 19122; 19697; 20198; 20200; 20194; 20203; 20339; 20750; 20779; 21812; 21813; 21823; 21825; 21844; 22059; 22223; 12818; 22325; 22350; 22359; 22370; 54401; 22627; 16975; 22142; 53761; 65969;
17123; 26903; 17165; 232889; 18412; 16801; 17164; 19099; 67847; 56705; 74097; 18777; 26754; 70601; 17064; 76779; 80888; 12301; 276919; 85031; 24069; 13063; 66586; 70024; 64177; 240047; 67177; 77018;
52521
mouse subset 3 (k)
17961; 17960; 11350; 76507; 11423; 11459; 11475; 11461; 11464; 71885; 11487; 11522; 11545; 11622; 11651; 627189; 11682; 16952; 12306; 11747; 11749; 11750;
11744; 11818; 11820; 11848; 27053; 11908; 11911; 11920; 11946; 11947; 12013; 12015; 12028; 12443; 12048; 110279; 12062; 12096; 12111; 545936; 12153; 12156;
12161; 12235; 12266; 12273; 110382; 12279; 12313; 12317; 12333; 12334; 12367; 12371; 12393; 12406; 12504; 12487; 21939; 12505; 12508; 626721; 12514; 16149;
12531; 12550; 12562; 12575; 12576; 12608; 243764; 12772; 12771; 12774; 12842; 12843; 12826; 12892; 11909; 12944; 12954; 12955; 12961; 12966; 26416; 12977;
12988; 104318; 12995; 13001; 13003; 13008; 66473; 13030; 13035; 13038; 13040; 13076; 13077; 13088; 13095; 72303; 13098; 56448; 13106; 13110; 13113; 337924;
13170; 13179; 13198; 18104; 13371; 107585; 13406; 13482; 99586; 15200; 30931; 13645; 13649; 13653; 208643; 50701; 15568; 13712; 13717; 13043; 328572; 269587;
13849; 13850; 13866; 13869; 13871; 13983; 104156; 14061; 14067; 109821; 58992; 74145; 14114; 14131; 14161; 110135; 14173; 99571; 14191; 14268; 14275; 14337;
14360; 14381; 14375; 14390; 622339; 14580; 14629; 14723; 14729; 54368; 12766; 14773; 14778; 14784; 14815; 227753; 14862; 14864; 14866; 14865; 14863; 14871;
107477; 14916; 14917; 15194; 15186; 12628; 15234; 217082; 15366; 15375; 15376; 15377; 15378; 225307; 15458; 94175; 193740; 193740; 14828; 69253; 15551; 15874;
15891; 15894; 15896; 15901; 15978; 16001; 16009; 16011; 29817; 16189; 16193; 16153; 16154; 16163; 16173; 16322; 54131; 16398; 16399; 16401; 16402; 104099;
16408; 16410; 16412; 16414; 16416; 16419; 16447; 16476; 16478; 16527; 18050; 16621; 16678; 268482; 16728; 16784; 16818; 16846; 16847; 16852; 107753; 19039;
16855; 16859; 233187; 16956; 16971; 14725; 17005; 17096; 56150; 17127; 17755; 17216; 17215; 17217; 17218; 17220; 17246; 26401; 26408; 17295; 17314; 17329;
110616; 17345; 214162; 83995; 17390; 17392; 17393; 17394; 17395; 17384; 17385; 17387; 17698; 17750; 66902; 17710; 17769; 17829; 623609; 17938; 27354; 18016;
18027; 18024; 18033; 18073; 68039; 18102; 18105; 18128; 18227; 209815; 18245; 18247; 18383; 18550; 18787; 50873; 268591; 18538; 18671; 18673; 18791; 18792;
18793; 18803; 18805; 18815; 18816; 18979; 18984; 18996; 19015; 19073; 18753; 18754; 320795; 18760; 26413; 26419; 26399; 19144; 19164; 19217; 19219; 19225;
14083; 19353; 218772; 19684; 19697; 19718; 69263; 106344; 53877; 20193; 20198; 20200; 20194; 20203; 20293; 20303; 20304; 20306; 20292; 20295; 20299; 56066;
16963; 20312; 20315; 20339; 20343; 20344; 67996; 13162; 20505; 104069; 20617; 20618; 20641; 545845; 20683; 20692; 20737; 20750; 20742; 20779; 70356; 20846;
20848; 20887; 20963; 21423; 22041; 22042; 21803; 21812; 21813; 21817; 21823; 21825; 21844; 21858; 21859; 110595; 21869; 21898; 21926; 21938; 22059; 22017;
17229; 22139; 22174; 22223; 22227; 22287; 243085; 80914; 12818; 22325; 22329; 22337; 22339; 22341; 22350; 22359; 22370; 22371; 22594; 74335; 22632; 54401;
22627; 16975; 22142; 12767; 65969; 17123; 17346; 232889; 85030; 18171; 18412; 54353; 29806; 16801; 17164; 20624; 16591; 382113; 19099; 23886; 19340; 18025;
67847; 320256; 12355; 20186; 56705; 74097; 51796; 100689; 18777; 380924; 15442; 67955; 26754; 72948; 67053; 73914; 57320; 70601; 69639; 19894; 17064; 64383;
72119; 76779; 74053; 80888; 12070; 16842; 85031; 13063; 394431; 66586; 64177; 194352; 66593; 56632; 240047; 64214; 70839; 27973; 77629; 67177; 66634; 77018;
52521; 68876; 66725; 142681; 242093; 11603
dog subset 1 (u)
610128; 609779; 403826; 479171; 415120; 607589; 475234; 477023; 477023; 476437; 403655; 476050; 480893; 611064; 477120; 479471; 403416; 403567; 483464; 490153; 403435; 403400; 478489;
479704; 480872; 486398; 476006; 611366; 487440; 478889; 403474; 442962; 480348; 477699; 475473; 487151; 476094; 442971; 609233; 607439; 482095; 475407; 403619
dog subset 2 (u)
481685; 476438; 403945; 478284; 403618; 484472; 486607; 486080; 609594; 487580; 607408; 608552; 403567; 488473; 487432; 442941; 403826; 403668; 607622;
611118; 476665; 477032; 486077; 483976; 403904; 483106; 487449; 611608; 481528; 477600; 403515; 607661; 476993; 475472; 403915; 475474; 481084; 403845;
490792; 474485; 483312; 479074; 403897; 403735; 403612; 480726; 480438; 478854; 609748; 403974; 612271; 403545; 403628; 403789; 403788; 608977; 609429;
609409; 482154; 478666; 479680; 403616; 450207; 449478; 474663; 481124; 478781; 611884; 487325; 403693; 476262; 609832; 480229; 485069; 478880; 487635;
478813; 403402; 488958; 608196; 403872; 480424; 477166; 442986; 489488; 480964; 484431; 477085; 487235; 484963; 606755; 482840; 485874; 403602; 611266;
475234; 403606; 403609; 403608; 490280; 403486; 488561; 477575; 486758; 477746; 607019; 485783; 403408; 404305; 475980; 477488; 483696; 484648; 442976;
476915; 475545; 403677; 476413; 491278; 403787; 480143; 475851; 483954; 490439; 403896; 403559; 486507; 612159; 485864; 481228; 403944; 490967; 477614;
403998; 481519; 481628; 477039; 485867; 487486; 403869; 490397; 612709; 490867; 403802; 494000; 477035; 476336; 476031; 489584; 480127; 481713; 483121;
476780; 488309; 483169; 611231; 477749; 480887; 478430; 475684; 611262; 476779; 489156; 612984; 488471; 486251; 490050; 480450; 488649; 480194; 481657;
476916; 607954; 486916; 479515; 486876; 483999; 474404; 612867; 481080; 490739; 491231; 609018; 484073; 484198; 474419; 477901; 610925; 486716; 479868;
482513; 484370; 484819; 490341; 612153; 609135; 607927; 489998; 475258; 488971; 484518; 609221; 477953; 474598; 475718; 478438; 485819; 476429; 490046;
485594; 477693; 485969; 489953; 486830; 491012; 607313; 476693; 480286; 483190
dog subset 3 (u)
491292; 480052; 477023; 489828; 483322; 403580; 608134; 488796; 403817; 403805; 490385; 403551; 403828; 403783; 491804; 490878; 403550; 610941; 483021;
479270; 481685; 607616; 403913; 475630; 476322; 403435; 478447; 476094; 479325; 482620; 479139; 482558; 479464; 489433; 442989; 442936; 478982; 476438;
403945; 403407; 486469; 442968; 403732; 403588; 474823; 403954; 483631; 486666; 483185; 475240; 486545; 612911; 474503; 479450; 403878; 403731; 403669;
478284; 483763; 611781; 481744; 610190; 403523; 488143; 449028; 403416; 488770; 403618; 488124; 607482; 403762; 403905; 484472; 486607; 486080; 477162;
490695; 491750; 609594; 487580; 607408; 608552; 403437; 478194; 489221; 479555; 479354; 611933; 474832; 483745; 480118; 479148; 482737; 403567; 403724;
487899; 486896; 488473; 487432; 488472; 403474; 485338; 612338; 607675; 486241; 403468; 477698; 488997; 480527; 403934; 442858; 480169; 611010; 489775;
474890; 403429; 481563; 490607; 478608; 609296; 483776; 403827; 403757; 485972; 489473; 486162; 486163; 442971; 484789; 442941; 403651; 403824; 403826;
478835; 403496; 403842; 403841; 478836; 403668; 481873; 607622; 611118; 476665; 442963; 478806; 479866; 607506; 403856; 611795; 403923; 477185; 477032;
486077; 483662; 488577; 608543; 479065; 608843; 475121; 494010; 415122; 415128; 479740; 415127; 484142; 483976; 403904; 610668; 485520; 483106; 607439;
479448; 487449; 609048; 610935; 489462; 475067; 403701; 487984; 611608; 489117; 485839; 487388; 403424; 403657; 404306; 481528; 480361; 477600; 403515;
480466; 491860; 610283; 609166; 474578; 442955; 480113; 404002; 484943; 403883; 612282; 403640; 403639; 488942; 488940; 607661; 404015; 476993; 478711;
491723; 487924; 479309; 476278; 486102; 483378; 476063; 475472; 403915; 475474; 481084; 403845; 490792; 608412; 478232; 475032; 474485; 403755; 476889;
480906; 480495; 486400; 403721; 478448; 609822; 481126; 403511; 611942; 442961; 483312; 478575; 474819; 475596; 479912; 611366; 477556; 490630; 487309;
475035; 487551; 403441; 482861; 475781; 481732; 609564; 442987; 403897; 485884; 478450; 480979; 403735; 403612; 480726; 479406; 611362; 478854; 609748;
403547; 403801; 610255; 442951; 483664; 488516; 491618; 403974; 403660; 403785; 403985; 612271; 403545; 403850; 403628; 403584; 488762; 488540; 489210;
403789; 403788; 608977; 442952; 484185; 609429; 609409; 482154; 478666; 477602; 477601; 479680; 403616; 450207; 403449; 449478; 403626; 481124; 478781;
486728; 477886; 475456; 480144; 610902; 481520; 490570; 611884; 480488; 487325; 403537; 403693; 478061; 480481; 476262; 476215; 403507; 442978; 415126;
491946; 477120; 489428; 403733; 403885; 403763; 609986; 403453; 494002; 403768; 478230; 403924; 485069; 476644; 607030; 478312; 611940; 490224; 613013;
608445; 478880; 487635; 478813; 442859; 486858; 480291; 474897; 475450; 475312; 403402; 488958; 479051; 477498; 403822; 608196; 610169; 403872; 484147;
478987; 476822; 484041; 488746; 475375; 403476; 612814; 609228; 480424; 477166; 442986; 442985; 489488; 487139; 480964; 403621; 403879; 484431; 476177;
488192; 477085; 487235; 484963; 606755; 482840; 476446; 485874; 403602; 611266; 485702; 611500; 611484; 403659; 475234; 403855; 475235; 489816; 489413;
490360; 484478; 403606; 403609; 403608; 490280; 403486; 610744; 494005; 490174; 478013; 479577; 474176; 477575; 486758; 477746; 607019; 612821; 478347;
611397; 488241; 485783; 403408; 404305; 475980; 485097; 403832; 442942; 475108; 610128; 611633; 486717; 477488; 611060; 486710; 479210; 483696; 403568;
484648; 477045; 486508; 476915; 483729; 479112; 489798; 477308; 478667; 477499; 478328; 403677; 487353; 480580; 476413; 476823; 474178; 403787; 480143;
475851; 483954; 403981; 489508; 608403; 490439; 487293; 477672; 489228; 482437; 403997; 490939; 487272; 491184; 486908; 484068; 476261; 403896; 478478;
403559; 476258; 403464; 486507; 488405; 611255; 485864; 481228; 403944; 488449; 490967; 490968; 486112; 475045; 477240; 477914; 479873; 485079; 484926;
477614; 403412; 477072; 403998; 490796; 481519; 481628; 477039; 485867; 487486; 403922; 403634; 477229; 403869; 478274; 490397; 490080; 612709; 480673;
490867; 490228; 607417; 487509; 403574; 474681; 403802; 494000; 477991; 477035; 476336; 399544; 609253; 480645; 475864; 476031; 487539; 489584; 483900;
474842; 403848; 403534; 610872; 476780; 488309; 477749; 480887; 478430; 488390; 475684; 609233; 489156; 612984; 611306; 611694; 488471; 478040; 477052;
479730; 490050; 479865; 489451; 488649; 480194; 481657; 476916; 475756; 610290; 488651; 486916; 476708; 479515; 486876; 483999; 608366; 485591; 609126;
607809; 488818; 612867; 477649; 481080; 403689; 491421; 480512; 490739; 608864; 608025; 475607; 609018; 611055; 483780; 474775; 612902; 490410; 484073;
484198; 474419; 477901; 487713; 610925; 474712; 486716; 488660; 477186; 479868; 490874; 489390; 476419; 484819; 477742; 488395; 612751; 612153; 607412;
609135; 479728; 477138; 477038; 607927; 489998; 486589; 403587; 492140; 475258; 488971; 484518; 482745; 609221; 612103; 482744; 477463; 491871; 474598;
485819; 480847; 476429; 490046; 485594; 442958; 477693; 485969; 477470; 489953; 486830; 491012; 607313; 476693; 474422; 484495; 480286; 480569; 483190;
613012; 486723; 608488; 611335
rat subset 1 (u)
rat subset 2 (u)
25377; 25728; 287774; 408242; 24223; 24888; 81613; 64625; 114496; 364403; 140932; 300811; 84480; 140923; 25402; 64044; 58918; 313173; 25412; 294337; 362584;
362285; 84353; 64529; 25311; 29139; 29167; 114214; 29515; 24330; 300253; 24331; 260320; 29243; 24366; 29348; 24367; 293860; 25661; 314322; 25019; 81504;
288077; 29424; 79240; 502373; 171016; 294254; 25617; 299331; 497838; 25462; 24494; 24499; 25205; 25325; 24516; 24518; 288001; 24525; 24530; 25608; 24536;
81680; 497722; 299858; 29409; 297894; 314856; 308106; 60331; 299626; 301458; 81735; 83619; 310738; 25494; 300303; 24605; 65051; 25737; 287422; 24644; 25344;
85243; 298696; 24650; 25692; 25738; 85253; 287527; 84024; 25664; 24668; 24672; 312754; 24674; 84400; 116590; 50689; 24686; 29192; 29674; 29677; 25622; 309151;
24703; 84509; 24708; 29288; 361568; 25271; 24615; 85247; 81778; 498209; 85385; 64301; 24786; 24790; 24791; 83805; 300095; 498351; 25125; 24822; 59086; 116487;
29591; 81810; 56083; 24842; 362788; 83785; 24874; 25696; 362558; 295238; 294250; 310678; 312492; 89816; 64647; 502913; 83507; 29647; 56042; 313929; 114558;
246756; 293104; 303068; 117271; 364420; 117279; 297101; 64520; 83631; 65040; 114505; 290364; 79252; 293524; 54299; 293017; 266601; 408200; 114552; 64547;
293852; 64845; 296554; 25514; 63885; 291005; 362891; 312916; 266709; 114766; 363544; 313819; 65274; 290652; 171574; 65199; 85311; 25309; 308913; 292759;
309434; 81918; 83765; 313876; 305230; 246273; 291279; 64345; 302963; 497835; 500348; 296468; 361648; 309380; 287474; 303517; 246142; 288916; 497816; 361994
rat subset 3 (u)
311860; 304883; 501072; 50681; 81822; 303742; 24170; 360354; 25026; 25370; 25591; 24177; 58968; 24179; 81638; 58850; 24185; 24186; 266802; 306283; 25377;
89805; 81641; 85333; 25380; 56611; 25291; 25673; 79125; 78963; 24205; 79116; 60371; 78971; 63879; 25649; 25728; 287774; 54226; 408242; 246097; 25385; 25240;
24208; 29221; 117273; 311193; 362456; 25242; 25612; 315305; 25389; 79255; 300711; 29693; 29600; 65262; 171374; 24223; 64639; 297994; 116502; 64557; 24887;
65036; 58919; 24224; 170929; 24888; 60434; 303836; 25295; 25181; 81613; 64625; 114496; 364403; 308755; 29373; 25296; 25644; 367786; 140932; 300811; 84480;
140923; 497672; 298566; 117517; 313421; 497751; 313495; 25678; 29153; 29154; 25403; 25166; 64314; 25402; 114555; 83584; 64026; 64044; 58918; 24248; 54236;
303747; 25073; 84349; 25406; 29187; 54237; 360621; 64465; 83502; 497718; 116777; 307618; 114851; 83571; 24252; 24253; 25410; 25301; 25423; 29195; 25229; 24260;
79557; 25590; 24261; 54240; 25753; 24854; 117029; 313173; 29393; 84352; 25412; 84032; 290905; 306628; 85250; 294337; 361821; 305104; 362584; 362285; 25654;
497663; 25419; 81649; 78965; 116630; 116549; 25307; 366219; 84353; 64529; 171293; 25424; 290257; 25425; 29175; 300047; 24296; 24297; 246070; 293985; 29277;
25086; 171352; 286929; 306722; 25311; 29139; 24310; 290484; 29167; 25112; 29467; 300710; 114214; 24312; 24314; 497670; 65135; 24318; 29238; 29515; 114856;
399489; 94204; 24323; 25313; 24329; 24330; 54318; 300253; 24331; 363854; 314436; 60465; 170915; 29452; 313052; 25315; 24335; 24336; 24337; 292673; 25149;
29251; 25439; 116677; 260320; 24946; 29243; 60327; 79451; 25598; 25440; 80841; 50646; 84490; 24366; 54250; 29348; 24367; 293860; 25661; 314322; 56718; 25320;
317385; 25150; 25019; 24383; 499156; 25172; 24387; 25235; 289481; 25283; 140589; 60449; 501069; 292804; 24404; 81504; 84027; 296654; 116686; 24421; 501110;
24423; 57298; 29438; 25260; 29487; 171528; 288077; 297893; 84577; 24443; 24446; 112400; 25059; 294274; 24451; 79240; 25735; 79256; 117282; 502373; 171016;
294254; 25617; 161476; 497838; 25462; 25587; 25585; 25712; 24482; 25718; 24483; 25662; 24484; 24494; 60582; 287287; 24498; 24499; 25205; 25325; 25670; 116996;
24504; 25118; 84598; 24514; 24516; 24518; 288001; 24525; 300242; 360625; 294853; 24530; 25608; 24536; 60584; 81680; 24539; 299858; 297604; 311337; 81515;
25671; 29357; 25631; 59328; 81516; 29409; 297894; 60430; 314856; 81518; 116667; 303604; 308106; 365057; 60427; 25332; 81683; 291234; 24590; 300339; 81686;
81687; 171052; 303413; 24565; 81709; 362657; 24577; 299626; 24585; 304447; 25017; 337868; 29148; 288770; 25135; 301458; 81735; 83619; 81736; 309452; 25493;
29508; 25336; 25337; 310738; 25494; 499194; 24599; 300303; 114593; 24605; 25054; 360265; 81738; 25601; 497733; 117254; 24617; 29432; 293992; 65051; 25737;
497724; 287422; 117276; 24644; 25154; 25344; 116589; 361241; 85243; 298696; 24650; 25692; 25738; 85253; 287527; 497831; 117540; 315713; 24664; 84024; 296851;
312086; 29441; 171068; 117058; 25664; 24668; 24672; 312754; 24674; 114906; 24678; 170538; 207122; 85420; 25522; 360748; 116590; 50689; 29513; 170851; 24683;
24686; 29192; 29674; 29677; 29497; 50557; 29527; 25614; 29222; 83801; 116689; 25622; 312703; 25532; 309151; 499870; 24703; 24706; 24708; 309165; 89809; 116468;
288414; 288003; 304528; 300807; 29288; 81771; 83840; 361568; 365320; 116644; 24615; 85247; 81778; 498209; 287562; 307947; 85385; 140941; 140593; 25280; 24778;
25551; 24779; 83511; 25553; 25549; 24904; 29504; 84386; 64301; 29219; 24786; 24787; 24790; 367846; 366126; 83805; 300095; 498351; 25124; 25125; 24918; 81801;
65189; 311622; 64525; 81807; 171046; 29338; 24822; 301965; 29436; 59086; 25717; 116487; 29591; 81810; 56083; 24835; 25625; 64550; 24842; 296099; 29268; 311786;
362788; 500363; 29261; 25232; 24860; 83529; 83785; 24874; 81818; 25696; 116669; 24883; 29753; 25578; 362558; 60628; 94342; 301265; 25379; 363518; 89816; 64647;
83507; 29647; 56042; 313929; 58945; 114558; 303068; 117271; 301133; 246775; 266610; 364420; 117279; 84578; 301164; 498883; 288583; 287101; 360481; 54190;
83631; 65040; 114505; 83504; 83500; 290364; 29702; 363509; 66015; 79252; 293524; 140665; 54299; 293017; 266601; 25243; 301062; 316064; 64535; 114552; 64547;
114114; 293852; 116698; 64845; 500929; 246118; 58834; 25514; 308416; 497739; 170724; 25491; 63885; 89776; 291005; 362891; 312916; 304135; 266709; 313323;
114766; 304962; 311546; 363544; 314465; 29392; 171071; 290652; 312682; 113961; 171350; 83527; 65199; 288584; 362228; 252902; 85311; 84582; 59301; 317589;
25309; 308913; 292759; 114246; 309434; 116469; 288753; 116493; 83765; 246273; 302668; 64345; 302963; 497835; 64803; 500348; 296468; 282845; 361648; 309380;
287474; 303517; 246142; 288916; 299828; 308457; 497816; 140734; 361994; 114491; 171018; 500448
mouse subset 1 (u)
19231; 15901; 12824; 12116; 56448; 13202; 18979; 113868; 113868; 54150; 11812; 625716; 28254; 28248; 20518; 18399; 19733; 11484; 17380; 11448; 12043; 12367; 12803; 20714; 14066; 12306; 13040; 13197;
11522; 109900; 14933; 103149; 14857; 14869; 14857; 18822; 15926; 12359; 14854; 15251; 232345; 17837; 14161; 23849; 11747; 12759; 56455; 13198; 268822; 13197; 53605; 14080
mouse subset 2 (u)
11699; 11816; 11818; 16617; 12010; 12048; 545936; 12122; 12124; 12143; 224630; 12175; 624862; 12177; 12367; 12370; 12371; 12804; 12824; 12833; 12840; 12841;
12813; 12845; 12387; 13030; 13176; 13179; 13191; 13347; 13498; 13653; 109901; 14068; 14058; 110135; 14178; 99571; 192176; 14268; 14281; 14375; 14784; 15163;
15194; 15370; 15461; 94175; 193740; 14828; 15519; 432551; 628438; 16176; 16194; 16195; 16153; 16399; 16416; 16419; 16476; 16478; 16542; 16644; 16653; 16816;
16846; 16847; 16880; 16906; 16971; 14725; 17136; 17180; 17246; 26407; 110616; 17873; 227197; 18007; 18024; 18049; 18073; 18176; 268591; 18538; 18626; 432633;
18673; 74769; 18708; 23988; 18763; 18791; 18803; 18815; 18816; 18979; 19016; 19045; 19052; 226849; 19055; 116847; 26413; 26417; 26419; 19094; 19122; 19164;
26444; 19182; 19247; 19367; 110157; 19384; 19645; 19744; 20091; 20130; 20181; 20198; 20200; 20194; 394252; 20248; 20416; 20595; 545845; 20683; 20692; 20779;
20788; 66661; 20848; 110213; 21803; 21810; 21812; 21813; 21817; 21825; 22059; 209456; 22029; 22031; 22339; 22346; 22354; 22359; 22668; 16975; 65111; 53761;
382523; 26903; 233489; 319182; 97122; 319158; 26430; 12361; 56041; 17977; 56208; 71609; 13626; 11492; 12123; 21933; 12633; 268930; 116870; 21945; 224997;
16600; 16432; 11504; 67384; 29810; 20185; 20874; 72567; 67952; 103135; 12049; 12125; 27061; 268566; 227613; 18777; 23984; 23882; 216440; 26754; 18769; 12034;
76779; 17776; 18226; 14232; 103967; 170770; 56461; 67220; 67118; 85031; 13063; 71984; 319594; 93742; 68585; 12211; 27428; 228775; 64113; 53417; 240047; 74498;
66813; 329581; 73658; 67573; 52700; 217169; 171543; 102060; 112407; 319161; 171388
mouse subset 3 (u)
11350; 11352; 113868; 11423; 11430; 11461; 71885; 11518; 11529; 11535; 11542; 11545; 11576; 11595; 11606; 11608; 11614; 11651; 11657; 11669; 11682; 11752;
11699; 11601; 16790; 11739; 16952; 12306; 11745; 11747; 11749; 11783; 11789; 11792; 11797; 11796; 11798; 11799; 11807; 11816; 11818; 11820; 16617; 14102; 14103;
11826; 11835; 11846; 11848; 228359; 11857; 11863; 27053; 11908; 11910; 11911; 11920; 11938; 381290; 11946; 11947; 12010; 12015; 12017; 12018; 12021; 12028;
12038; 12443; 12043; 12047; 12048; 12050; 12053; 110279; 12096; 12111; 545936; 12122; 12124; 12143; 12144; 12156; 12159; 12161; 12169; 224630; 12175; 624862;
12177; 12189; 12259; 110382; 12279; 230099; 12305; 12333; 12334; 12380; 12362; 12366; 12367; 12363; 12368; 12369; 12370; 12371; 12359; 12507; 12516; 20778;
21947; 12505; 12515; 12534; 23834; 12540; 12550; 12558; 12560; 12562; 12575; 12576; 12606; 12608; 110794; 12611; 13032; 12643; 12669; 12671; 11435; 11438;
11443; 11447; 11449; 12759; 12774; 12804; 12842; 12843; 12824; 12825; 12826; 12827; 12828; 12829; 12832; 12833; 12834; 12839; 12840; 12841; 12813; 12814; 12845;
12870; 11909; 12914; 12944; 12970; 26416; 12977; 12981; 12995; 13010; 58214; 12387; 13030; 13033; 13034; 13035; 13036; 13038; 66445; 13076; 13077; 13095; 72303;
72082; 13106; 13113; 337924; 69635; 13176; 13179; 11421; 13190; 13191; 13197; 13198; 77591; 13347; 13361; 18104; 13385; 64705; 13489; 13490; 13498; 19252;
13555; 230857; 13614; 13645; 13649; 13653; 13665; 109901; 15568; 13712; 13043; 13805; 328572; 13819; 269587; 13849; 13856; 13857; 13866; 13870; 13982; 13983;
14061; 14062; 14063; 14068; 14071; 14058; 74145; 11770; 14079; 16204; 12140; 19229; 14104; 14156; 110135; 14173; 14178; 99571; 14198; 192176; 14268; 14281;
14284; 56717; 14459; 233908; 14360; 14375; 622339; 14453; 14460; 14580; 14598; 14600; 14626; 14629; 14632; 14695; 54214; 14751; 14775; 14784; 56637; 227753;
14782; 14862; 14864; 14871; 14872; 14939; 15163; 433759; 15182; 15186; 15234; 211323; 15277; 14998; 217082; 15368; 15370; 15378; 225307; 15387; 15461; 94175;
193740; 14828; 624853; 69253; 432551; 628438; 15902; 15903; 15978; 16000; 16001; 16002; 16008; 16009; 16176; 16181; 16189; 16193; 16194; 16195; 16153; 16168;
16170; 16322; 16399; 16416; 16419; 16451; 16452; 16476; 16478; 16542; 16644; 16653; 110310; 268482; 16668; 16816; 16846; 16847; 16878; 16880; 16956; 16971;
14725; 17000; 17005; 17096; 17125; 17126; 17127; 17129; 17131; 17136; 17180; 17210; 17246; 17258; 17261; 26401; 26406; 26407; 26408; 17311; 17314; 17319; 17342;
17345; 54601; 17380; 83995; 17390; 17395; 17386; 17523; 17250; 17685; 17750; 17769; 17869; 17873; 17897; 17904; 17918; 17927; 17928; 17938; 17975; 227197;
18007; 18024; 18033; 18034; 18035; 18044; 18045; 18046; 18049; 18073; 68039; 18125; 18126; 18159; 18176; 18212; 18214; 18390; 18550; 18477; 18787; 224105;
18504; 268591; 18538; 18596; 18626; 170768; 432633; 18667; 18671; 18673; 20724; 20723; 74769; 18708; 23988; 18763; 18791; 18793; 18803; 18815; 18816; 26432;
433328; 18854; 18976; 18979; 330260; 269823; 18984; 18985; 18986; 18987; 19016; 19045; 19052; 226849; 19055; 18767; 18768; 18753; 109333; 18761; 18762; 19090;
26413; 26417; 26419; 19094; 26415; 26395; 19106; 19109; 19122; 19164; 26444; 19182; 19205; 19211; 19225; 14083; 19231; 69202; 15170; 19247; 19305; 19341; 19367;
19361; 110157; 218772; 19411; 19645; 19697; 19687; 19718; 69263; 106344; 19883; 20091; 20111; 20130; 20133; 104001; 20198; 20200; 20194; 394252; 20248; 20293;
26398; 56086; 20416; 20437; 20439; 20449; 27401; 20525; 20527; 20533; 20538; 15567; 214084; 20517; 20519; 20568; 20595; 20617; 545845; 20656; 20682; 20683;
20687; 20375; 20779; 20788; 66661; 20846; 20848; 20850; 20855; 56274; 58231; 67923; 67673; 21423; 21672; 110213; 21752; 22041; 21788; 21803; 21809; 21810;
21812; 21813; 21817; 21825; 21926; 21937; 21969; 22059; 27223; 209456; 17229; 22029; 22030; 22031; 22147; 22171; 22174; 22227; 22333; 22339; 22346; 22352;
22354; 22359; 22371; 22431; 22627; 22631; 22668; 22785; 16975; 12767; 224727; 27226; 11737; 56292; 233489; 319182; 319158; 26430; 12361; 56041; 27413; 17977;
56455; 56208; 11492; 12123; 50930; 22035; 14082; 21933; 12633; 15183; 18519; 17191; 26433; 268930; 83945; 54189; 21945; 224997; 16600; 16591; 19879; 16432;
13666; 73341; 240913; 11504; 29810; 19340; 20185; 20874; 72567; 67952; 11864; 208194; 102448; 108737; 14181; 12049; 12125; 74006; 27061; 21849; 19192; 268566;
22143; 22152; 50768; 100689; 18777; 53333; 53319; 67628; 26405; 18008; 23984; 108114; 23882; 216440; 26754; 23794; 18769; 101739; 12034; 226641; 72119; 76779;
21981; 23821; 17872; 14232; 94044; 70231; 18458; 170770; 13169; 56461; 50932; 66437; 67877; 235661; 67220; 67118; 85031; 56233; 58991; 70415; 13063; 71984;
64177; 319594; 194352; 66593; 54645; 68585; 228775; 70008; 53417; 240047; 74498; 70839; 66813; 329581; 80751; 73658; 67573; 52700; 217169; 171543; 102060;
68876; 112406; 112407; 319161; 68097; 171388; 67379; 56222; 269800; 68857
dog subset 1 (vl)
481576; 610868; 612911; 489188; 479597; 485540; 492281; 403974; 403985; 403850; 480255; 442942; 479564; 609604
dog subset 2 (vl)
607439; 477747; 475032; 483312; 476881; 481328; 488126; 612271; 403545; 478906; 478905; 485209; 609429; 609409; 477886; 610902; 442859; 610981; 477166; 487235; 609956; 403426; 403602; 479483;
403987; 479158; 476915; 483729; 448786; 482986; 403869; 490532; 481330; 474845; 477534; 490006; 478040; 442949; 486242; 477412
dog subset 3 (vl)
477699; 474487; 483322; 478321; 403410; 490385; 403910; 403168; 403783; 475251; 490878; 479476; 477753; 489458; 607616; 479464; 442936; 403407; 486469;
403588; 403954; 475240; 486545; 474503; 479450; 449028; 403416; 488770; 403618; 403658; 403461; 403762; 491750; 489450; 403503; 611933; 442947; 403724;
488473; 403980; 487746; 487044; 489369; 483845; 486564; 607076; 403646; 487438; 612608; 403756; 403469; 403468; 489476; 479329; 488997; 403934; 491325;
474890; 403429; 477165; 477796; 487225; 484791; 403826; 403467; 445450; 478806; 403856; 611795; 403923; 481276; 608246; 477032; 486077; 483662; 415122;
415128; 494003; 479353; 403904; 610668; 607439; 478767; 485839; 484199; 403424; 403657; 404306; 481528; 487870; 442980; 480466; 492129; 477709; 404002;
403883; 612282; 493995; 476105; 477747; 403641; 488942; 488940; 490153; 476063; 475472; 488808; 475474; 442938; 478232; 481367; 478969; 475032; 474485;
480906; 442957; 403943; 483312; 478575; 611366; 477556; 476881; 490630; 485828; 611148; 403441; 480348; 479182; 442987; 490657; 485742; 484434; 478450;
482372; 486073; 403735; 403812; 475124; 480438; 403975; 609779; 481558; 403801; 610255; 488516; 482839; 403782; 403974; 481328; 488126; 403660; 403989;
403870; 403785; 403985; 612271; 403545; 612582; 403850; 478906; 478905; 611642; 403628; 610823; 611309; 403977; 403976; 442990; 488762; 481837; 403796;
478432; 485209; 484990; 475134; 609429; 609409; 403984; 403811; 478666; 480173; 479680; 478151; 403616; 403449; 481124; 607183; 481712; 475720; 486728;
477886; 481360; 610902; 476196; 487612; 490570; 479260; 403537; 403693; 403438; 403507; 489428; 403733; 403445; 489432; 489429; 403885; 611789; 403763;
403823; 486335; 403768; 483653; 477024; 611315; 483925; 485930; 486495; 478813; 442859; 484519; 480558; 403822; 403784; 490455; 403872; 610981; 484391;
482025; 611921; 448778; 403476; 607885; 477166; 403879; 607205; 483951; 481772; 487235; 609956; 480048; 482840; 403426; 476446; 488167; 479473; 403602;
403606; 482196; 486295; 490904; 494005; 609934; 490722; 479577; 477575; 486758; 477746; 607019; 474652; 478347; 476104; 403408; 478703; 403740; 610774;
403797; 403589; 485919; 403544; 442942; 403987; 403779; 490563; 490255; 609852; 403955; 481656; 477045; 479158; 476915; 483729; 484454; 403494; 487353;
476823; 490463; 491615; 480749; 476879; 403650; 448788; 403981; 480600; 448786; 403522; 491148; 448790; 611927; 482986; 449622; 480840; 403999; 403994;
489508; 489228; 482437; 489640; 403561; 483066; 611255; 485864; 490967; 485088; 482771; 486303; 403703; 403998; 488596; 481628; 607489; 610625; 477841;
448807; 482905; 403417; 488605; 403922; 403634; 487437; 403869; 490080; 480673; 490867; 480019; 403968; 608979; 403982; 486588; 403802; 482932; 399544;
478902; 490532; 481330; 483900; 610538; 475230; 483724; 477534; 485022; 476512; 490006; 611694; 488471; 403482; 481708; 442949; 474636; 403625; 611678;
484822; 480698; 478950; 487323; 488653; 608025; 477278; 445454; 608707; 479479; 489449; 611355; 486124; 489390; 609119; 492016; 608988; 489925; 486549;
490265; 478837; 609203; 481110; 403502; 608094; 477412; 490512; 607858; 478564; 609101; 485238; 479619; 490576; 475830
rat subset 1 (vl)
50655; 365601; 25389; 307403; 81503; 24333; 301382; 24494; 24498; 361042; 29527; 25541; 60325
rat subset 2 (vl)
29467; 306274; 25150; 81504; 361580; 25663; 25466; 24499; 25205; 54258; 29385; 54259; 65038; 24516; 24518; 81515; 25631; 81736; 29541; 25737; 29741; 25619; 85253; 363633; 314615; 24695; 25676;
24708; 309165; 116637; 25216; 313121; 24842; 24884; 79426; 117022; 361796; 310678; 360972; 252971; 84578; 89829; 360801; 170465; 363278
rat subset 3 (vl)
24153; 79250; 50681; 29650; 29316; 25591; 24176; 81722; 24179; 25690; 24185; 287454; 25290; 81639; 89805; 60371; 54226; 246097; 24208; 117273; 25612; 315305;
79255; 300711; 58919; 24224; 170929; 24888; 25245; 24225; 25295; 367786; 29681; 298657; 313495; 316010; 25166; 114555; 64044; 25404; 50662; 362489; 500985;
114494; 315291; 60350; 25660; 497809; 25668; 171369; 84349; 25406; 287435; 25599; 54237; 64465; 362110; 114851; 83571; 24253; 24258; 24259; 289032; 309361;
310071; 57301; 25412; 301012; 24267; 25419; 81649; 78965; 116630; 497822; 25610; 84353; 64529; 171293; 246070; 29277; 25086; 25147; 79128; 29139; 24310; 29467;
25253; 399489; 315219; 306792; 24323; 25313; 24329; 24330; 117045; 299606; 363854; 302811; 24334; 170915; 24335; 24337; 292673; 291703; 306274; 360635; 29251;
25439; 116677; 25584; 290757; 171075; 84490; 24366; 54250; 84489; 24367; 294515; 499148; 56718; 25449; 25319; 25320; 317385; 25150; 25019; 25172; 81664; 60429;
81504; 114105; 171551; 84027; 29438; 25260; 361580; 171528; 25734; 29199; 24446; 29560; 25675; 24451; 25098; 25099; 25100; 79256; 297099; 79242; 502373; 25461;
299331; 25464; 25261; 298205; 24481; 25712; 24482; 25662; 84351; 24493; 24494; 25663; 25466; 60582; 116562; 25704; 287287; 24498; 24499; 25205; 294797; 54258;
29385; 24500; 25325; 117539; 360303; 84405; 64546; 116553; 116996; 301289; 29197; 245920; 54259; 65038; 24954; 64831; 24516; 24518; 83731; 64030; 288001;
24530; 313050; 25608; 60584; 299858; 25008; 361795; 59107; 297604; 81515; 171577; 25631; 50554; 367100; 81516; 24548; 60430; 314856; 60427; 81683; 300339;
81686; 171045; 25335; 63849; 81687; 117033; 171052; 81707; 498194; 301059; 85482; 307231; 311658; 366998; 83619; 81736; 81525; 191575; 24599; 24600; 24603;
24605; 29541; 84595; 25341; 289747; 29665; 24617; 29431; 64513; 25737; 288937; 266775; 24649; 29741; 24653; 25692; 25619; 25096; 25097; 85253; 25664; 246358;
83803; 24680; 170538; 29340; 81749; 25522; 116590; 50689; 50658; 170851; 25268; 314615; 29192; 117263; 25526; 25637; 81752; 24929; 84023; 25527; 497767; 29527;
24695; 56813; 117063; 24699; 114767; 497784; 363875; 298012; 24706; 25676; 24708; 309165; 365510; 24716; 81771; 365320; 94195; 25742; 25122; 287562; 116637;
81780; 287561; 29397; 29538; 60665; 25216; 24772; 501550; 25544; 25651; 24778; 65197; 81826; 366126; 83805; 25125; 362896; 314621; 24886; 24817; 64678; 59086;
81809; 29591; 81811; 312510; 361655; 310553; 29260; 289337; 24835; 25625; 497808; 24842; 29268; 311786; 362788; 311245; 25360; 116484; 304944; 25361; 24873;
83785; 114111; 116669; 363247; 24884; 79426; 117022; 60628; 306817; 286926; 25445; 360972; 171137; 65137; 252971; 266610; 117279; 84385; 294235; 89829;
361321; 246253; 64565; 29455; 59103; 363984; 116673; 65035; 246118; 83820; 170465; 362505; 64537; 286904; 58974; 360557; 24934; 306009; 29392; 500054; 117108;
282817; 315298; 363983; 155140; 363278; 170704; 289076; 171415; 311865; 170816; 64862; 287362; 499069; 310681
mouse subset 1 (vl)
11428; 11793; 11910; 12978; 14825; 13806; 22592; 16176; 16193; 74551; 19225; 20280; 18788
mouse subset 2 (vl)
13198; 319955; 14360; 14784; 14884; 16177; 16180; 16194; 16195; 227288; 12765; 16331; 16332; 16476; 16478; 17096; 17127; 18033; 18207; 18538; 18708; 18709; 18792; 18815; 20020; 19152; 19227;
218397; 19645; 19697; 20303; 20969; 26409; 22059; 22612; 22695; 16178; 193742; 382523; 107829; 12703; 15183; 12702; 20602; 52538; 77040
mouse subset 3 (vl)
232345; 11429; 11430; 11487; 11541; 11545; 11555; 11596; 11606; 11622; 11651; 11684; 11689; 11687; 11601; 11797; 11799; 11820; 14102; 11835; 11848; 27053;
11908; 11911; 11920; 12443; 12043; 12047; 12048; 12062; 12064; 12096; 12169; 12261; 12353; 230099; 12796; 12362; 12363; 12370; 12389; 12395; 12402; 12428;
12455; 12475; 12487; 21941; 21949; 12494; 21939; 21947; 12505; 12514; 16149; 12534; 12540; 107951; 12575; 12576; 12608; 12609; 12652; 12653; 71884; 12675;
71991; 12768; 12824; 12836; 12846; 11909; 12944; 26416; 12977; 12981; 12984; 12985; 12387; 13030; 13033; 13095; 72082; 13106; 13075; 13132; 13179; 11421; 13198;
13482; 13555; 72962; 13610; 13614; 13645; 13649; 13653; 13684; 50701; 15568; 56501; 13807; 328572; 13856; 13866; 13870; 13871; 13872; 319955; 18612; 14061;
14062; 14063; 14066; 109821; 14128; 14156; 110135; 14173; 14184; 99571; 56484; 14256; 56717; 14309; 14319; 14459; 233908; 13349; 14360; 14375; 14460; 14678;
14683; 14784; 20310; 330122; 56637; 14871; 14884; 14939; 15162; 15216; 15234; 15251; 15357; 15368; 15375; 15376; 15377; 15405; 15446; 15461; 15466; 15499;
15519; 15894; 15901; 15974; 15977; 15978; 16000; 16008; 16150; 16175; 16176; 16177; 16180; 16181; 16183; 16184; 16189; 16193; 16194; 16195; 16196; 16197;
227288; 12765; 16199; 16153; 16154; 16156; 16159; 16160; 16163; 16170; 16171; 16173; 15945; 16331; 16332; 16337; 64602; 16476; 16478; 16531; 16590; 16644;
16774; 16816; 16818; 16846; 16878; 16971; 16992; 16994; 268977; 17000; 17096; 17075; 17127; 17128; 17130; 17131; 17194; 17210; 17246; 17295; 17311; 17319;
83995; 17390; 17392; 17393; 17394; 17395; 17381; 17386; 17387; 433938; 17750; 623609; 17874; 27354; 18018; 18019; 18022; 18024; 18033; 18036; 18102; 18126;
18127; 18160; 18176; 18207; 18220; 18383; 18413; 18439; 18787; 18479; 114774; 18538; 18671; 20725; 241197; 18712; 18708; 18709; 18783; 18791; 18792; 18793;
18805; 18806; 18815; 19016; 57349; 19079; 18750; 18753; 18754; 18755; 18762; 19091; 26413; 26417; 26419; 19094; 26420; 26395; 19123; 19152; 19164; 19179; 19215;
19216; 19217; 19218; 19219; 19223; 19224; 19225; 19227; 19228; 19255; 19264; 19267; 19283; 19353; 19359; 218772; 218397; 19645; 19697; 19698; 19713; 20111;
20133; 20190; 20202; 20203; 20209; 20210; 20276; 20293; 20303; 20304; 20306; 20292; 20297; 20311; 20969; 20315; 66226; 20339; 20344; 26398; 27401; 20525; 56485;
56643; 20515; 20375; 20779; 20848; 20852; 20869; 21391; 21405; 22042; 21803; 21808; 21812; 21832; 21841; 21843; 24088; 142980; 21898; 53791; 21926; 21937;
21938; 22059; 17229; 22030; 22031; 622602; 22034; 22095; 22166; 80914; 22329; 22337; 22339; 22341; 22371; 22596; 22612; 22695; 16178; 12767; 110052; 21789;
14283; 107829; 56505; 12703; 14082; 12633; 18171; 15937; 12702; 67526; 11450; 231382; 23886; 64292; 56489; 20970; 12355; 22152; 320558; 52538; 20301; 15442;
106648; 56486; 195046; 27007; 213053; 23821; 101185; 237038; 76898; 234836; 66824; 26934; 329244; 53945; 58181; 83430; 81897; 66809; 77040; 225192; 64214;
67841; 96979; 170639; 67443; 216799; 446099; 69585
dog subset 1 (ae)
483397; 483318; 489368; 477029; 479353; 476447; 484922; 486513; 477601; 611278; 490695; 480454; 484822; 403665; 481563
dog subset 2 (ae)
607768; 489077; 478484; 475359; 478650; 485839; 487341; 480348; 477210; 477601; 478781; 480144; 610902; 484622; 403693; 403924; 481884; 484963; 606755;
607140; 486613; 482840; 482083; 403907; 609934; 477746; 478464; 475980; 490439; 485864; 481628; 477039; 487437; 488593; 493997; 476521; 611171; 477237;
480645; 489818; 475864; 607289; 611262; 488649; 479515; 486242; 608937; 480931; 483399; 484373; 491614; 477593; 482331; 484500; 478802; 442943; 607991;
485041; 478082; 490521; 490776; 611615; 478877; 491076; 403518
dog subset 3 (ae)
490385; 490878; 479476; 489486; 476322; 482558; 480487; 403670; 612911; 479450; 477101; 403523; 449028; 403416; 403905; 487097; 486127; 477162; 487826;
474944; 477270; 607768; 489077; 478484; 403437; 403567; 487899; 486896; 487366; 489369; 485338; 611782; 608847; 484610; 611695; 607898; 475992; 488997;
442858; 481131; 609920; 474890; 403429; 475359; 611216; 609767; 607139; 403826; 403739; 490269; 479441; 403856; 609266; 487649; 477185; 610344; 484142;
483106; 610935; 476715; 482996; 478650; 485839; 611243; 611103; 403424; 403657; 404306; 481528; 487870; 475590; 608908; 491860; 403883; 612282; 493995;
476105; 403640; 483324; 486102; 487341; 403845; 475032; 478170; 449477; 403873; 403943; 487351; 483312; 611366; 403441; 403432; 480348; 403735; 480726;
403979; 609779; 482839; 478800; 489208; 488437; 477956; 403788; 483318; 477210; 609429; 444857; 486522; 486513; 490976; 477601; 442954; 475882; 480034;
491145; 480124; 478781; 475456; 480144; 610902; 476196; 481520; 490570; 489652; 489368; 403693; 478061; 476262; 403438; 442978; 415126; 480229; 477019;
489432; 611597; 494002; 484768; 444854; 403768; 474729; 403924; 480646; 481884; 480618; 442859; 608196; 403872; 484666; 488669; 477166; 477889; 484963;
606755; 482840; 476446; 487491; 482083; 403907; 494001; 490021; 490904; 494005; 609934; 484903; 477575; 486758; 477746; 607019; 478464; 612821; 478347;
489547; 485004; 475896; 475980; 442942; 484922; 481656; 483420; 480526; 477045; 476915; 483729; 478328; 491786; 607023; 403999; 489508; 490439; 490939;
486940; 486407; 486507; 612159; 608433; 487501; 485864; 612861; 478845; 485079; 403412; 607720; 481519; 481628; 477039; 474355; 403634; 487437; 477229;
403869; 489620; 490867; 607417; 478874; 486315; 488593; 403802; 494000; 477991; 484721; 493997; 609253; 476521; 478902; 611171; 477237; 480645; 489818;
475864; 485850; 607289; 482346; 611262; 482048; 477302; 442949; 488649; 490263; 489943; 403625; 489497; 479515; 486242; 612510; 487524; 608844; 608937;
480931; 482039; 479336; 477322; 607929; 484373; 491614; 477593; 481247; 610682; 485782; 478848; 482331; 478372; 486549; 484500; 491909; 612797; 477639;
478802; 442943; 485041; 478082; 487490; 490546; 490521; 480632; 490776; 491076; 403518; 482208; 609341; 491356; 613012; 475280
rat subset 1 (ae)
293618; 25724; 78967; 79128; 84495; 361381; 294853; 117038; 25296; 114495; 29455; 292770
rat subset 2 (ae)
81507; 310914; 140590; 54238; 303811; 399489; 287114; 79113; 29560; 305506; 294853; 29357; 25631; 312538; 314856; 24577; 315840; 298696; 24650; 304822; 287925; 25692; 64204; 368070; 29340;
25272; 29677; 85385; 83805; 313121; 29591; 81810; 497808; 290805; 312560; 24919; 56011; 29753; 56010; 25576; 25578; 117275; 246756; 83631; 25495; 54299; 360801; 362293; 293619; 308576;
300266; 266803; 361526; 259271; 306455; 363334; 365661; 307594; 314310; 367090; 301446; 84686; 308516
rat subset 3 (ae)
25591; 24185; 287454; 25380; 79116; 64310; 64373; 25389; 300711; 24887; 58919; 24224; 25181; 307151; 83470; 29373; 25667; 315824; 25644; 85272; 81507; 310914;
140590; 497672; 25402; 83584; 64026; 156726; 500985; 64033; 25729; 365367; 64315; 54237; 83502; 94201; 114483; 114851; 83571; 246060; 25164; 54238; 140583;
116563; 25412; 294027; 289395; 25420; 81649; 315707; 116549; 266729; 306722; 29167; 24314; 84350; 444984; 444985; 303811; 399489; 307794; 116651; 287114;
24323; 25313; 24329; 24330; 117045; 116636; 366142; 314436; 170915; 24337; 292673; 291703; 303687; 50646; 79113; 25661; 25150; 29319; 316033; 60429; 313017;
81504; 29438; 24446; 58947; 29560; 502373; 25617; 24471; 25261; 84351; 114517; 170921; 360606; 296456; 24511; 25724; 305506; 24516; 24525; 300250; 294853;
360626; 309368; 140433; 298941; 117036; 303369; 60374; 25671; 29357; 25631; 50554; 59328; 81516; 292083; 78967; 314856; 116667; 308106; 25332; 60331; 497834;
25335; 24561; 81709; 24566; 300999; 24577; 65261; 315840; 24592; 81736; 300303; 24605; 81528; 304947; 25737; 29237; 298696; 24650; 25692; 85240; 64204; 368070;
29240; 302934; 24680; 170538; 29340; 29355; 116590; 50689; 25272; 29513; 170851; 303200; 363855; 25581; 29677; 83499; 29527; 361381; 298012; 84600; 24705;
24706; 24708; 309165; 300807; 501560; 60352; 25544; 85385; 24779; 361825; 24790; 24791; 266602; 311331; 83805; 501099; 367264; 313121; 171046; 301965; 361178;
116487; 29591; 81810; 83580; 25625; 497808; 64550; 24842; 362675; 362788; 29261; 301442; 304577; 83785; 24874; 81818; 64566; 290805; 24883; 312560; 363247;
24919; 56011; 29753; 56010; 25576; 25578; 117275; 246756; 65154; 295052; 89829; 83631; 25495; 246253; 114523; 54299; 360801; 308016; 300219; 362293; 78969;
114031; 24373; 288444; 363140; 308576; 300266; 503215; 113910; 363228; 266803; 363067; 315298; 361526; 317396; 84357; 366975; 259271; 363334; 365661; 296078;
363294; 307594; 286993; 314310; 298342; 84686; 308516; 303181; 170520; 192126; 300455
mouse subset 1 (ae)
192897; 84004; 432548; 13132; 22594; 19358; 16691; 16668; 17751; 12159; 26399; 23886; 20733
mouse subset 2 (ae)
12166; 12167; 12168; 12580; 13544; 13555; 13560; 14191; 15251; 16418; 16668; 14725; 17126; 17127; 17216; 17246; 17869; 17920; 23988; 18763; 18772; 67451; 18791;
18810; 18949; 18754; 26419; 26414; 19182; 20416; 20779; 26409; 21812; 21813; 21938; 22427; 22591; 22632; 54401; 22627; 22628; 22629; 22631; 12667; 71609; 21945;
18121; 20185; 20602; 59026; 56460; 59047; 64451; 12419; 66042; 55942; 65964; 66408; 70925; 106766; 59079; 225164; 52708; 72774; 241066; 217124; 52428
mouse subset 3 (ae)
11545; 11651; 11684; 11686; 16952; 11792; 11840; 11852; 11910; 11920; 12028; 12443; 12043; 12111; 12151; 12153; 12156; 110075; 12160; 12161; 12162; 12166;
12167; 12168; 12189; 53414; 12367; 12368; 12369; 12399; 12402; 12444; 12445; 12447; 12527; 12478; 12476; 12534; 12550; 12567; 12571; 12575; 12576; 12577; 12580;
12581; 12649; 11773; 12824; 12821; 12902; 12955; 26416; 12982; 12988; 12995; 13131; 69635; 13191; 18104; 13433; 13435; 13436; 109620; 13544; 13555; 104394;
13559; 13560; 13614; 13645; 13649; 13653; 13684; 13685; 13711; 13712; 328572; 13866; 13870; 13871; 13872; 13982; 14027; 19229; 14191; 14198; 14268; 14360;
14603; 12091; 14683; 55948; 14784; 15234; 54426; 15251; 15461; 14828; 15507; 15901; 16150; 16403; 16398; 16400; 16410; 16412; 16416; 192897; 16418; 16476;
16653; 16678; 110308; 16691; 16668; 16669; 16773; 16776; 16777; 226519; 16882; 16905; 14725; 17125; 17126; 17127; 17128; 17129; 17131; 17199; 84004; 17246;
26401; 26407; 17295; 17314; 17342; 110616; 17350; 17393; 268395; 17685; 15235; 19882; 17750; 66902; 17869; 17913; 17920; 18015; 18033; 18176; 18294; 18514;
18538; 18619; 23988; 18763; 18791; 18793; 18796; 18810; 18949; 18970; 19041; 18750; 18753; 18754; 320795; 26413; 26417; 26419; 19094; 26414; 26415; 26395;
26397; 26400; 19181; 19182; 19185; 19225; 19358; 19359; 19373; 19401; 218772; 19645; 19697; 19883; 110651; 29857; 20339; 26398; 20416; 20533; 20583; 20587;
20683; 20692; 20730; 20732; 20779; 20807; 20849; 26409; 21423; 21752; 21781; 21810; 21812; 21813; 21824; 21937; 21938; 21969; 22059; 22062; 22031; 56191; 22171;
22218; 22256; 22339; 22346; 22352; 22418; 22427; 22431; 22591; 74335; 22596; 22632; 54401; 22627; 22628; 22629; 22631; 14563; 12667; 13411; 71609; 22402; 12427;
12702; 21945; 18121; 17164; 11450; 18208; 20185; 20602; 229933; 329504; 78733; 59026; 211770; 83554; 14313; 15505; 56289; 59047; 64451; 12419; 13143; 26434;
56363; 66042; 20466; 26934; 55942; 54609; 58194; 223828; 65964; 66408; 106766; 59079; 214230; 65960; 225164; 216965; 52708; 67050; 217124; 52428; 22416;
114886; 69601; 67379; 73230
dog subset 1 (d)
485730; 480454; 474890; 487113; 476419; 490193; 489814; 478605; 442987; 403782; 609429; 475666; 485773; 403486; 415125; 612911; 479303; 482840; 612820; 477977; 480222; 474709; 488729;
489908; 481708; 484822; 487247; 479368; 403647; 486596; 489301; 478853; 486337; 607998; 485204; 486934; 482670; 481557; 403693; 481981; 477965; 484500; 403810; 475463; 608532; 475223;
448804; 606811; 484037; 489204; 479515; 487151; 474503; 483066; 477953
dog subset 2 (d)
475537; 403429; 481563; 479866; 477185; 474844; 476745; 607439; 480472; 488148; 608080; 477295; 609164; 487309; 480348; 403170; 488126; 609429; 484927;
609409; 480144; 610902; 476196; 403693; 478939; 415126; 491946; 448784; 403924; 485069; 611940; 613013; 476725; 488693; 403692; 478813; 484920; 442859;
477498; 477166; 481772; 484963; 403426; 485874; 611484; 403907; 482686; 478109; 403753; 480533; 491303; 609091; 479577; 477575; 486758; 477746; 607019;
474652; 478464; 480338; 481014; 403955; 490863; 476915; 478127; 483729; 479949; 475080; 476672; 490457; 485650; 608403; 489610; 476710; 486548; 403559;
486507; 612159; 611255; 488449; 490967; 478845; 491950; 611193; 477966; 481224; 489932; 608918; 483343; 480525; 403869; 489620; 481598; 485406; 478874;
486588; 611171; 607083; 481330; 479854; 483724; 478385; 479955; 481319; 608556; 477480; 478350; 485022; 475684; 480381; 478040; 477052; 477302; 486251;
480147; 476891; 490263; 481657; 478340; 479884; 477664; 480354; 479648; 481627; 477216; 475266; 487323; 607662; 612913; 480233; 485643; 490941; 487343;
480134; 489442; 483728; 611951; 612902; 478023; 484198; 481295; 477901; 608946; 474677; 483335; 476189; 489012; 481997; 486403; 608367; 484819; 480709;
491230; 478372; 609080; 488970; 485823; 611862; 489756; 607927; 488836; 484776; 478802; 476136; 478300; 477011; 478553; 475465; 476871; 483716; 477953;
477864; 485819; 489511; 477251; 480949; 480060; 488176; 484651; 491076; 610340; 611936; 612305; 488237
dog subset 3 (d)
477699; 480425; 491292; 403580; 478250; 477236; 490385; 484375; 486344; 403828; 485769; 475251; 490878; 474808; 476600; 479476; 403435; 482558; 442936;
486469; 442968; 403588; 485189; 607262; 475240; 486545; 612911; 474503; 479450; 477101; 487718; 483763; 611781; 610190; 403523; 449028; 403416; 403618;
612349; 488124; 403461; 481947; 475867; 477162; 491750; 403437; 475526; 475966; 403516; 484724; 610342; 491030; 486563; 609361; 480416; 476694; 474684;
403567; 487746; 487044; 487366; 489369; 485338; 475007; 611782; 608847; 484610; 478442; 486564; 476126; 403931; 487438; 488997; 484780; 403934; 442858;
480169; 611010; 481131; 475537; 609920; 491325; 474890; 403429; 481563; 475359; 611216; 609346; 486028; 490607; 610263; 488891; 477796; 442971; 489104;
480578; 484791; 484790; 608910; 607922; 478806; 479866; 403856; 611795; 477185; 478108; 474844; 488922; 612240; 479991; 477032; 486077; 489664; 415128;
494003; 476745; 483106; 476067; 607439; 480472; 480032; 489573; 610935; 489128; 478767; 607887; 492057; 485839; 611243; 403657; 404306; 481528; 480361;
487870; 481565; 480466; 491860; 488574; 480113; 403883; 488508; 403640; 403639; 489287; 487761; 488148; 488942; 490153; 476993; 474468; 476063; 475473;
491901; 607724; 608459; 403915; 477340; 612454; 475582; 488808; 489095; 608080; 608169; 403727; 403845; 490792; 484445; 608412; 609642; 449024; 475032;
488340; 480906; 484626; 480495; 479885; 490156; 403873; 481126; 478581; 489469; 483312; 494007; 478047; 478575; 475597; 609164; 492246; 479074; 487309;
475035; 478100; 403441; 480348; 609531; 403170; 442946; 442987; 479864; 485884; 607578; 478450; 480979; 403735; 403612; 474680; 403979; 403909; 403975;
609779; 403801; 610255; 442951; 484067; 488516; 610316; 479122; 482839; 403974; 488126; 403660; 403989; 403785; 489957; 403790; 403985; 403850; 478906;
442990; 475574; 478381; 483665; 478800; 481500; 486493; 477956; 608977; 609423; 484185; 609429; 484927; 609409; 403758; 487743; 482154; 475624; 608024;
486513; 490978; 480173; 404021; 479193; 480124; 481124; 479072; 607183; 483736; 477886; 612436; 612631; 480144; 610902; 476196; 490570; 403451; 475770;
480218; 403537; 403693; 478939; 612973; 479155; 478061; 480481; 476262; 403438; 609832; 479417; 489428; 403733; 403885; 494002; 474729; 483653; 481662;
483995; 403924; 482978; 485069; 611940; 613013; 476725; 611315; 485677; 488693; 608738; 480021; 483925; 485930; 479552; 479678; 480272; 403692; 478813;
485061; 484920; 442859; 486858; 480291; 477498; 403822; 480676; 483148; 474472; 490404; 484147; 476749; 478343; 611921; 448778; 488746; 475375; 403476;
609604; 607885; 481417; 480424; 477166; 475041; 476828; 479875; 403621; 484431; 478684; 481772; 484963; 403426; 476446; 487491; 476034; 485874; 403602;
489971; 476636; 611484; 490013; 403907; 480869; 478109; 403753; 490360; 481756; 403606; 606861; 403609; 403557; 478053; 491303; 479248; 609027; 612559;
612199; 489082; 490904; 489968; 494005; 609934; 490722; 478686; 484903; 609091; 479577; 474176; 477575; 486758; 478311; 489532; 477746; 607019; 474652;
478464; 612821; 478347; 489547; 485004; 485783; 480338; 481014; 487336; 403832; 403740; 403544; 442942; 475108; 610128; 475509; 477488; 403955; 490863;
484648; 474539; 480526; 486508; 476915; 480175; 477221; 478127; 474606; 483729; 479112; 488666; 403565; 479949; 475080; 476672; 487353; 403787; 490457;
485028; 607023; 403676; 485650; 403650; 489972; 489973; 403981; 480600; 448790; 449622; 403999; 403994; 489508; 608403; 476549; 612817; 403687; 489610;
403748; 482437; 492299; 482838; 476710; 486407; 486548; 403559; 483042; 486507; 488405; 611255; 485864; 612861; 488449; 490967; 478845; 490968; 491950;
611017; 611193; 485056; 485079; 477966; 481224; 403412; 488213; 607720; 403998; 489932; 481628; 608918; 474355; 477841; 483343; 474759; 403922; 480525;
403869; 490397; 489620; 480673; 490867; 492072; 485406; 403973; 609156; 474536; 612742; 478874; 608979; 403982; 486588; 403802; 490848; 399544; 481625;
493997; 611171; 489398; 607083; 488559; 481330; 479854; 474842; 483724; 485915; 478385; 479747; 486271; 481319; 607683; 475451; 608556; 478350; 485022;
480887; 478430; 488125; 475684; 480381; 482346; 607359; 484718; 478040; 477052; 488199; 477302; 488529; 442949; 486251; 487057; 611442; 477857; 476891;
490263; 481657; 489943; 403683; 479884; 483299; 482101; 485657; 480354; 488651; 482192; 481453; 484822; 610825; 479648; 481627; 477216; 475266; 486242;
487323; 482530; 607662; 487524; 612913; 480233; 485643; 474494; 490941; 610835; 403689; 487343; 609864; 484513; 482049; 479465; 480134; 489442; 483728;
611951; 612902; 481233; 478023; 609003; 479177; 483107; 445454; 484198; 474851; 481295; 477901; 476617; 484948; 474675; 480287; 486338; 477082; 608946;
610349; 477202; 481809; 474677; 483335; 490729; 476319; 485773; 482166; 476189; 479409; 490874; 481997; 474434; 403420; 479254; 486403; 486981; 608367;
484819; 611329; 480709; 491230; 478372; 480595; 477305; 609080; 477818; 485823; 486549; 477629; 611862; 403598; 478974; 489756; 607927; 488836; 484776;
478802; 476136; 478300; 487586; 477011; 479036; 475465; 490283; 476871; 483716; 477847; 477953; 482744; 477864; 487490; 609390; 480632; 485819; 489511;
477251; 480949; 480060; 481414; 607858; 491225; 611457; 488176; 475811; 491839; 484651; 486438; 478331; 477199; 479741; 612601; 491076; 487187; 610637;
478879; 611936; 606799; 486723; 488237; 483315
rat subset 1 (d)
rat subset 2 (d)
140908; 83571; 24253; 25301; 497663; 116549; 64391; 25112; 29467; 287765; 294924; 313582; 84482; 294515; 81659; 81674; 297893; 29560; 499092; 25466; 24516;
24517; 24518; 29357; 25631; 50554; 54267; 314856; 304798; 24577; 299626; 337868; 288770; 308690; 360610; 83619; 81524; 81736; 316241; 25737; 24649; 298696;
25619; 25738; 315713; 368070; 289758; 361365; 25517; 497991; 362102; 85421; 25522; 116590; 50689; 50658; 25272; 29670; 363875; 362787; 24708; 81758; 309165;
81763; 290641; 295213; 316164; 307947; 171379; 363002; 24786; 24790; 24791; 366126; 25124; 25125; 367264; 317256; 313121; 117526; 25705; 366964; 84574;
316742; 316099; 24842; 362675; 500450; 292925; 301442; 24873; 24919; 25576; 313666; 117022; 360471; 25445; 304157; 310678; 360908; 316369; 501403; 498183;
300772; 171137; 313929; 29419; 84578; 301164; 113894; 295052; 83422; 317399; 114505; 315769; 116501; 312849; 25414; 83633; 362251; 500131; 116673; 363287;
361251; 299258; 501506; 84580; 60571; 192218; 291259; 291005; 362859; 312916; 302582; 304286; 298604; 303135; 309757; 314930; 155430; 94165; 499882; 290652
296639; 311796; 363067; 500870; 307940; 366232; 293660; 307799; 114845; 301009; 295426; 311926; 499884; 170837; 303968; 293165; 171444; 81918; 361666;
246273; 282826; 362271; 365755; 312649; 84686; 116674; 310615; 364681
rat subset 3 (d)
24153; 24795; 311860; 81822; 29275; 24165; 25591; 308578; 29663; 58968; 29146; 25690; 24185; 25374; 287454; 56611; 79116; 246097; 25385; 24208; 25387; 25388;
25612; 315305; 25389; 79255; 300711; 304127; 64639; 297994; 64557; 24887; 58919; 24224; 24888; 303836; 24225; 309871; 116599; 29373; 367786; 497672; 114486;
29618; 295703; 24239; 29716; 25399; 50688; 25297; 58942; 24242; 64202; 81715; 25402; 367218; 50662; 362489; 156726; 500985; 114839; 64033; 25729; 29157;
315291; 304758; 24932; 497809; 25406; 54237; 171102; 64465; 83502; 497718; 116777; 94201; 140908; 114483; 362110; 114851; 83571; 246060; 25164; 54238; 289993;
66019; 24252; 24253; 25410; 257649; 309361; 24854; 116561; 54241; 57301; 60463; 113936; 81646; 497663; 54245; 25419; 81649; 78965; 116549; 307641; 114122;
29382; 24962; 84353; 64529; 79129; 25086; 25147; 64391; 29167; 64470; 25112; 29467; 287765; 304859; 298298; 24314; 307483; 360509; 25253; 314616; 83514;
399489; 307794; 25313; 24329; 24330; 54318; 117045; 497806; 363854; 314436; 304786; 25437; 170915; 25315; 24337; 59323; 170909; 25149; 24356; 304063; 294924;
29251; 25439; 25584; 29243; 290757; 315191; 84490; 361969; 363460; 25317; 54250; 60662; 29348; 25444; 25443; 84489; 25114; 313582; 294515; 54251; 25661;
314322; 308411; 292409; 25319; 83826; 25150; 81657; 81659; 25172; 25159; 362125; 24387; 27100; 24388; 316033; 140589; 81666; 192243; 303248; 81504; 24408;
24413; 84027; 306516; 81674; 363519; 29424; 297893; 84577; 294673; 24446; 29560; 289623; 499092; 117062; 24451; 79239; 25735; 83498; 79256; 117282; 502373;
29577; 294254; 266759; 24471; 24476; 25464; 25261; 304988; 360415; 25712; 24482; 25718; 25151; 25662; 25285; 297767; 84351; 24494; 25466; 60582; 116562;
287287; 25084; 24497; 24498; 54258; 116553; 66029; 306626; 24506; 114517; 24508; 291100; 315346; 24511; 60583; 24514; 24516; 24517; 24518; 65195; 25471; 25048;
85245; 24525; 303530; 83781; 116641; 60374; 299858; 116565; 25008; 83838; 81515; 294446; 362391; 29357; 25631; 50554; 81516; 54267; 25750; 171347; 291439;
60430; 314856; 304798; 29386; 116667; 303604; 308106; 315606; 300339; 81686; 81687; 81709; 81725; 498982; 24577; 298894; 299626; 337868; 288770; 85482;
300955; 308690; 307231; 311658; 25492; 361400; 305897; 360610; 83619; 29228; 81524; 81736; 309452; 25493; 316241; 24599; 25496; 29492; 59109; 25054; 25502;
289747; 29665; 497733; 117254; 24617; 60325; 29431; 64513; 500242; 116782; 65051; 25737; 58934; 116551; 81745; 25154; 25344; 116459; 24649; 298696; 25619;
85240; 29322; 25738; 85253; 25515; 316009; 315713; 302915; 368070; 361365; 25517; 171068; 25682; 25664; 24668; 360258; 60660; 362102; 24675; 171378; 29748;
65179; 29471; 24680; 25023; 170538; 29340; 81749; 84006; 29355; 85421; 25522; 360748; 116590; 50689; 114509; 50658; 25272; 29513; 170851; 303200; 363855;
24686; 314615; 29670; 58949; 50557; 25526; 497767; 29527; 25614; 29222; 24924; 25622; 363875; 362787; 24703; 294429; 24705; 24708; 291787; 81758; 305584;
309165; 89809; 29480; 312937; 81763; 290641; 81771; 24615; 295213; 64196; 60352; 362890; 316164; 25122; 24767; 24768; 287562; 29538; 24772; 25544; 25651;
307947; 500281; 494445; 361814; 140593; 29483; 366432; 24778; 29502; 171379; 361825; 363002; 499417; 287993; 24786; 313845; 24790; 367846; 366126; 83805;
501099; 25124; 25125; 367264; 24918; 317256; 313121; 302711; 117526; 24816; 171046; 25705; 366964; 301965; 306862; 361178; 59086; 84574; 29591; 316742; 83580;
361655; 313494; 316099; 24835; 24842; 362675; 311786; 362788; 140933; 292925; 25653; 85489; 116484; 58819; 81920; 301442; 304944; 25361; 24873; 83785; 362779;
116669; 313235; 290805; 24919; 25576; 353227; 313666; 192675; 29619; 117022; 360471; 94342; 25445; 84017; 84584; 304157; 310678; 288471; 316369; 361181;
307766; 501403; 300772; 171137; 29647; 56042; 246334; 313929; 29419; 246575; 498576; 84578; 301164; 291076; 295052; 501164; 89829; 362485; 361662; 317399;
114505; 29282; 116501; 59114; 300057; 117186; 66015; 305253; 287899; 29455; 25414; 83633; 252917; 362251; 500131; 360801; 116673; 313539; 363287; 361251;
299258; 300084; 501506; 84580; 171497; 116698; 140666; 292756; 299923; 363014; 60571; 192218; 315159; 291259; 360161; 500228; 362505; 291005; 361798; 362859;
312916; 252919; 315461; 300475; 314126; 114212; 302582; 25482; 282840; 304286; 65096; 116464; 316228; 303135; 299151; 309243; 24654; 170641; 367072; 314465;
314930; 84016; 155437; 155430; 94165; 499882; 290652; 313874; 296639; 311796; 363067; 114512; 500870; 64031; 366232; 315298; 300193; 293660; 56817; 116479;
307799; 114845; 301009; 311926; 499884; 363087; 170837; 308775; 313825; 60462; 293165; 308365; 171444; 309016; 81918; 116469; 361666; 296078; 171114; 286993;
246273; 282826; 362271; 312516; 289076; 296551; 292534; 365755; 295323; 302511; 312649; 287944; 315776; 311567; 362393; 84686; 307482; 289083; 116674;
303580; 171018; 364681; 316758
mouse subset 1 (d)
56706; 26399; 12575; 12296; 13047; 22057; 17872; 99633; 105387; 215114; 628035; 15368; 16175; 16476; 18263; 18795; 19055; 14183; 11910; 12450; 18791; 105445;
12238; 69190; 18028; 57444; 69234; 15937; 23886; 71721; 13803; 75646; 20393; 23938; 67760; 13663; 236010; 209683; 78610; 207278; 330662; 76089; 230393; 17246;
328525; 54004; 225115; 55942; 77125; 80877; 72446; 18647; 27280; 53618; 192231; 15939; 64009; 218639; 20185; 23849; 11504; 11911; 13017; 20515; 93742
mouse subset 2 (d)
12568; 12576; 12608; 12611; 12914; 12995; 13001; 13144; 13197; 13198; 13207; 14013; 14201; 56458; 56484; 231580; 68705; 433759; 15251; 433238; 16180; 16476;
16477; 16478; 17126; 17127; 17128; 17132; 17246; 17248; 17342; 54601; 17829; 17869; 17873; 17927; 17938; 76936; 17984; 18023; 18024; 18032; 18033; 18037; 18125;
18538; 18712; 23988; 18792; 18803; 18854; 18949; 18972; 20021; 18736; 19014; 110854; 18760; 18762; 26413; 26417; 26419; 19094; 26420; 26414; 19167; 53380;
19353; 26448; 19645; 19651; 19697; 627490; 76808; 20196; 20230; 56086; 20481; 20586; 83797; 545845; 20683; 20692; 20375; 20846; 20848; 20849; 270627; 26409;
21374; 21417; 21411; 21804; 21815; 619653; 21922; 21973; 22059; 22062; 22004; 22088; 546265; 22218; 22337; 22632; 22629; 22642; 16178; 252870; 14283; 268903;
382523; 12545; 17974; 12418; 17165; 56469; 17977; 18222; 15183; 18519; 18412; 12427; 17344; 56200; 17164; 16600; 56404; 65962; 108098; 12927; 242425; 170791;
20970; 208727; 140580; 27277; 353187; 56805; 15184; 242687; 23897; 18432; 81601; 26405; 66505; 23882; 216197; 26754; 245671; 231876; 56381; 20362; 107817;
83814; 93759; 22762; 15214; 104248; 58521; 14232; 68379; 58202; 20466; 105837; 244667; 19700; 11854; 56513; 67220; 16599; 80987; 65964; 59006; 94217; 58800;
20623; 73916; 50754; 20679; 107975; 93742; 66165; 228775; 68626; 140579; 18744; 621205; 81004; 381802; 217124; 170947; 319161; 81703; 22761; 74343; 218215
mouse subset 3 (d)
232345; 20716; 11350; 11461; 11464; 11486; 11545; 11771; 11764; 11595; 16449; 11622; 11651; 17025; 11655; 11684; 12306; 11792; 11799; 14102; 14103; 11835;
109689; 216869; 27053; 11908; 11910; 11911; 11920; 12013; 12015; 12017; 12021; 12028; 12443; 12043; 12048; 12051; 12053; 12064; 12142; 109778; 12156; 12169;
12189; 12226; 12258; 12288; 12289; 12293; 12295; 12297; 12298; 12313; 12317; 12328; 12367; 12393; 12395; 12399; 12402; 51813; 12444; 12445; 12447; 12452; 12455;
72949; 12504; 21941; 12505; 12534; 12530; 12540; 12550; 12558; 12560; 12567; 12568; 12571; 107951; 12575; 12576; 12577; 12580; 12581; 72391; 12591; 12606;
12608; 110794; 108000; 12648; 12675; 12759; 74325; 67300; 12768; 12772; 56373; 12912; 11909; 12914; 12928; 12944; 26416; 12977; 12995; 13000; 13001; 13003;
13016; 107869; 12387; 13030; 13057; 13106; 13075; 13144; 13191; 13194; 13197; 13198; 13207; 13211; 74754; 18104; 13367; 94176; 13482; 13496; 14605; 13555;
104394; 13645; 13649; 13653; 13665; 13684; 15569; 15568; 13712; 13714; 13726; 328572; 13849; 13866; 13869; 13876; 13982; 13983; 23871; 23872; 14013; 14061;
14062; 14066; 14058; 109821; 14114; 14156; 14161; 14163; 14164; 14173; 14176; 14178; 14180; 14165; 14182; 14184; 14186; 14201; 14225; 56484; 14254; 14268;
14281; 14282; 14284; 14293; 14319; 26423; 14360; 54393; 231580; 14460; 14461; 14536; 14580; 11692; 14581; 12091; 14632; 14682; 224139; 216860; 14784; 14810;
14815; 56637; 68153; 68705; 15161; 15194; 433759; 15182; 15212; 15234; 15251; 53323; 433238; 15361; 15368; 15369; 15378; 15384; 15387; 15461; 15205; 193740;
15525; 15507; 15874; 15894; 15901; 15951; 52668; 15978; 16000; 16001; 16004; 16008; 16011; 19664; 16150; 16176; 16180; 16181; 16183; 16189; 16190; 16191; 16193;
227288; 16163; 15930; 26356; 16334; 16403; 16362; 16364; 16402; 16412; 16419; 16447; 16452; 16476; 16477; 16478; 56543; 16509; 16542; 16621; 16647; 16653;
16691; 16666; 16774; 16854; 56048; 16905; 16971; 16976; 16992; 16998; 17096; 17118; 17119; 17126; 17127; 17128; 17131; 17132; 109731; 232087; 17190; 17210;
17246; 17248; 17257; 17260; 26401; 26406; 26407; 17295; 214162; 83995; 17390; 17395; 17685; 66902; 623609; 18198; 17863; 17869; 18109; 17873; 17927; 17938;
76936; 27354; 17973; 17984; 18018; 18019; 18027; 18021; 73181; 18023; 18024; 18029; 18032; 18033; 18034; 18035; 18037; 18125; 18126; 18128; 18129; 18211; 18212;
18245; 18247; 18413; 18439; 18550; 18477; 18787; 18788; 18479; 114774; 23983; 268591; 18538; 18567; 228026; 18607; 18667; 18673; 20713; 18712; 23988; 18792;
18793; 18796; 18797; 18803; 18815; 18817; 235611; 18854; 54128; 18949; 18968; 20021; 18736; 18986; 19015; 19016; 622262; 19045; 627816; 72930; 110854; 19056;
19057; 19060; 19065; 18750; 18751; 18753; 18754; 18755; 18759; 320795; 18760; 18762; 19090; 26413; 26417; 50772; 23939; 26419; 19094; 26420; 26414; 26415;
26395; 26397; 26400; 19122; 19152; 19167; 53380; 19204; 19211; 19215; 19224; 19225; 14083; 19231; 19242; 19247; 19353; 26448; 110157; 19386; 19401; 19411;
19645; 225182; 19650; 19651; 19696; 19697; 19687; 19736; 19888; 627490; 76808; 20111; 20198; 20196; 224903; 29857; 67125; 20230; 20276; 20277; 20278; 20293;
20297; 20315; 20339; 20344; 26398; 56086; 74729; 20382; 20383; 20439; 20481; 20512; 242607; 20525; 20537; 20516; 20586; 20587; 83797; 20610; 170930; 545845;
20662; 20683; 20687; 20375; 20779; 20807; 20846; 20848; 20849; 20850; 270627; 26409; 21372; 21374; 21390; 21423; 21417; 21411; 21752; 21418; 21781; 21803;
21804; 21812; 21815; 21824; 21843; 619653; 21894; 21922; 21926; 21973; 22059; 209456; 22062; 22030; 22031; 22067; 22088; 22094; 22150; 22166; 50493; 56550;
66105; 546265; 22218; 80914; 22329; 22337; 22339; 22367; 22371; 107684; 22427; 22632; 22629; 235320; 22642; 140475; 12227; 16178; 252870; 224727; 14283; 15364;
17979; 268903; 382523; 100683; 72399; 17974; 99375; 93762; 12418; 56469; 12361; 56041; 22061; 17977; 18222; 13411; 22035; 60406; 15209; 15183; 18519; 19134;
12427; 12570; 12702; 12448; 17082; 12237; 56200; 17164; 16600; 65019; 65962; 26941; 79456; 24056; 240913; 545532; 216725; 23886; 12927; 242425; 217166; 170791;
20602; 20970; 230674; 208727; 329504; 140580; 27277; 353187; 56438; 56805; 15184; 27219; 21849; 242687; 53901; 70584; 17988; 22601; 23897; 18432; 81601; 26405;
57259; 66505; 59126; 17768; 20301; 23882; 110147; 216197; 26754; 76257; 30806; 16568; 217578; 50883; 245671; 17532; 107503; 231876; 330814; 56406; 78309;
20362; 107817; 319565; 271564; 18795; 319387; 83814; 69632; 21981; 22762; 74053; 15213; 15214; 104248; 170755; 58521; 14232; 240168; 68379; 58202; 20466;
56321; 105837; 18569; 19700; 26934; 223870; 11854; 80906; 16456; 56513; 67220; 16599; 80987; 65964; 94217; 58800; 320528; 20623; 76375; 50754; 381319; 20679;
269870; 107975; 207425; 93742; 194352; 66165; 214230; 29811; 216965; 228775; 68626; 140579; 18744; 621205; 23885; 64214; 107733; 66078; 81004; 229644; 67395;
381802; 69009; 70549; 16396; 67235; 69538; 217124; 319262; 72823; 319161; 81703; 71059; 22761; 269800; 218215; 76265; 16772

### B. Additional Liver Toxicity Datasets

Additional datasets generated in rat, mouse, and dog have been similarly evaluated. These include data on TCDD (dioxin) toxicity in mouse liver reported in Boverhof, et al. (2005) “Temporal and dose-dependent hepatic gene expression patterns in mice provide new insights into TCDD-Mediated hepatotoxicity” Toxicol. Sci. 85:1048-1063; on acetaminophen (APAP) toxicity (hl) in rat liver reported in Heinloth, et al. (2004) “Gene expression profiling of rat livers reveals indicators of potential adverse effects” Toxicol. Sci. 80:193-202; on a number of compounds (ez) including dimethylnitrosamine, 2-nitrofluorene, aflatoxin B1, and 4-(methylnitrosamino)-1-(3-pyridyl)-1-butanone in rat liver reported in Ellinger-Ziegelbauer, et al. (2004) “Characteristic expression profiles induced by genotoxic carcinogens in rat liver” Toxicol. Sci. 77:19-34; and lipopolysaccharide (LPS) toxicity (hg) in dog liver reported in Higgins, et al. (2003) “Gene expression analysis of the acute phase response using a canine microarray” Toxicol. Sci. 74:470-484.

Table 3A lists Entrez gene ID numbers for primate counterpart biomarker genes derived from these datasets based on liver toxicity studies. Human and chimpanzee counterparts are identified, and other species can be similarly listed where sufficient information is available on the genome. The human subset 1 provides counterpart human genes, expressly listing the Entrez gene ID, the accepted symbol, and a short description of the gene corresponding to the marker. The primate subset 2 are Entrez gene IDs of markers which are reported to interact directly (e.g., by physical association or 2-hybrid interaction) with markers of subset 1, either reported from human or another species counterpart. The primate subset 3 are Entrez gene IDs of markers which have been associated by being referenced in a published abstract with one of the markers of subset 1.

Table 3B lists Entrez gene ID numbers for counterparts in selected non-primate species. These are provided in dog, rat, and mouse. Similar counterparts can be generated for other species, as their genome sequences become more complete and counterpart equivalents can be determined.

Table 4 lists markers which are found from analysis of multiple datasets. These markers have independent studies which commonly point to them as important, but not limited only to liver toxicity. Similar analyses can be performed for the additional markers, including those in Tables 5A/B and 6A/B.

Particularly preferred discriminatory markers are those which are listed in the various lists of Table 4, including combinations thereof, for evaluating or testing toxicity pathways. These are human markers which different independent studies commonly indicate are relevant discriminatory markers in liver toxicity. Primate counterparts will also be important, as the primate test systems are most likely relevant to clinical trials.

Other preferred embodiments are the Table 3A subset 2 and 3 for primate markers from each of the respective studies. The markers may be selected individually or in combinations within or across the studies (e.g., one or more from different datasets). Combinations of one or more of these markers can be generated from others from other tables. The Table 2A subsets 2 and 3 are similarly preferred, being primate markers. Individual markers, or combinations comprising markers from Table 2A subset 1 and Table 3A subset 1 are of interest, including combinations across various table subsets. Combinations including one or more markers from, e.g., the Table 3A subsets 2 and 3 with a member of Table 3A subset 1, or Table 2A or 2B, are also valuable.

Markers from non-primate species may also be important, e.g., Tables 2B and 3B. Individual or combinations within or across the subsets 2 and 3 are preferred. Combinations across the subsets 1 also have value.

### C. Supplementary Liver Toxicity Datasets

Additional datasets generated in rat and mouse have been similarly evaluated, but not limiting them as markers only to liver toxicity. These include data on furan toxicity (f) in rat liver reported in Hamadeh, et al. (2004) “Integration of clinical and gene expression endpoints to explore furan-mediated hepatotoxicity” Mutat. Res. 549:169-183 (PMID 15120969); methapyriline toxicity (mp) in rat liver reported in Hamadeh, et al. (2002) “Methapyrilene toxicity: anchorage of pathologic observations to gene expression alterations” Toxicol. Pathol. 30:470-482 (PMID 1218793); various carcinogen compounds (i) on mouse liver reported in Iida, et al. (2005) “Unique patterns of gene expression changes in liver after treatment of mice for 2 weeks with different known carcinogens and non-carcinogens” Carcinogenesis 26:689-699 (PMID 15618236); and paclitaxel compounds (1) on rat liver reported in Lee, et al. (2004) “cDNA microarray gene expression analysis and toxicological phenotype for anticancer drug” J. Vet. Med. Sci. 66:1339-1345 (PMID 15585946). Data is presented in Tables 5A/B and 6A/B as described above, analogous to Tables 2A/B and 3A/B, with various preferred embodiments derived from subsets 3 and 2, and 1

### D. Additional Toxicity Datasets

Additional datasets have been identified and analyzed, besides the liver toxicity datasets described above. In particular, these include data on mouse aortic vascular smooth muscle cells (VSMC) in culture exposed to benzo[a]pyrene (BaP), a polycyclic aromatic hydrocarbon present in tobacco smoke inducing oxidative stress (j), which is reported in Johnson, et al. (2003) “Genomic profiles and predictive biological networks in oxidant-induced atherogenesis” Physiol. Genomics 13:263-275 (PMID: 12657712); data on cardiotoxicity responses to exposure to ditoxin, doxorubicin, isoproterenol, and/or LPS (cd) reported in Hirakawa, et al. (2005) “Method for determining cardiotoxicity” US Pat App 20050138675; a cardio polymorphisms dataset (cpm) reported in Cargill, et al. (2005) “Genetic polymorphisms associated with cardiovascular disorders and drug response, methods of detection and uses thereof” US Pat App 20050272054. A neurotoxicity dataset (dm) from sarin treatment of rats is reported in Damodaran, et al. (2006) “Gene expression profiles of the rat brain both immediately and 3 months following acute sarin exposure” Biochem. Pharmacol. 71:497-520 (PMID: 16376859). Two rat kidney toxicity datasets have been included, responses to treatment with sevoflurane products (k) reported by Kharasch, et al. (2006) “Gene expression profiling of nephrotoxicity from the sevoflurane degradation product fluoromethyl-2,2-difluoro-1-(trifluoromethyl)vinyl ether (“Compound A″) in rats” Toxicol. Sci. 90:419-431 (PMID 16384817); and with ochratoxin A (u) reported by Luhe, et al. (2003) “A new approach to studying ochratoxin A (OTA)-induced nephrotoxicity: expression profiling in vivo and in vitro employing cDNA microarrays” Toxicol. Sci. 73:315-328 (PMID 12700408). Analysis is included of human PBMC response to cigarette smoke condensate compounds (vl) as reported in van Leeuwen, et al. (2005) “Differential gene expression in human peripheral blood mononuclear cells induced by cigarette smoke and its constituents” Toxicol. Sci. 86:200-210 (PMID 15829617). Human keratinocyte culture response to various compounds (ae) is also analyzed, as reported by Bae, et al. (2003) “Gene expression patterns as potential molecular biomarkers for malignant transformation in human keratinocytes treated with MNNG, arsenic, or a metal mixture” Toxicol. Sci. 74:32-42 (PMID 12773770). And response of rat lung is analyzed (d) based on data reported by Dillman, et al. (2005) “Genomic analysis of rodent pulmonary tissue following bis-(2-chloroethyl) sulfide exposure” Chem Res Toxicol. 18:28-34 (PMID: 15651846).

These data are presented in Tables 5A/B and 6A/B, as in Tables 2A/B and 3A/B. Markers described in these tables, individual or in combinations, and including members of the various subsets, are embodiments of the invention. The markers are not limited to toxicity in the organ, cell type, or pathway where identified, e.g., the markers may be valuable in toxicity in other organs, cell types, or pathways.

Table 5A lists Entrez gene ID numbers for primate counterpart biomarker genes derived from these datasets based on toxicity studies. Human and chimpanzee counterparts are identified, and other species can be similarly listed where sufficient information is available on the genome. The human subset 1 provides counterpart human genes, expressly listing the Entrez gene ID, the accepted symbol, and a short description of the gene corresponding to the marker. The primate subset 2 are Entrez gene IDs of markers which are reported to interact directly (e.g., by physical association or 2-hybrid interaction) with markers of subset 1, either reported from human or another species counterpart. The primate subset 3 are Entrez gene IDs of markers which have been associated by being referenced in a published abstract with one of the markers of subset 1.

Table 5B lists Entrez gene ID numbers for counterparts in selected non-primate species. These are provided in dog, rat, and mouse. Similar counterparts can be generated for other species, as their genome sequences become more complete and counterpart equivalents can be determined.

Other preferred embodiments are the Table 5A subset 2 and 3 for primate markers from each of the respective studies. The markers may be selected individually or in combinations within or across the studies (e.g., one or more from different datasets). Combinations of one or more of these markers can be generated from others from other tables. The Table 5A subsets 2 and 3 are similarly preferred, being primate markers. Individual markers, or combinations comprising markers from Table 5A subset 1 and Table 6A subset 1 are of interest, including combinations across various table subsets. Combinations including one or more markers from, e.g., the Table 3A or 6A subsets 2 and 3 with a member of Tables 3A or 6A subset 1, or Table 2A or 2B, are also valuable. Combinations of markers from various Tables and subsets are incorporated herein.

Markers from non-primate species may also be important, e.g., Tables 2B, 3B, 5B and 6B. Individual or combinations within or across the subsets 2 and 3 are preferred. Combinations across the subsets 1 also have value.

### V. Pattern ID from Data

Genomic profiling platforms have allowed collecting huge quantities of data from individual samples. With whole genome expression profiling, measurements of expression levels of most genes can be generated, providing, e.g., some 30K data points per sample. However, the interpretation of the expression data to a physiological outcome or response is difficult to achieve. If, e.g., only 10 organs were involved in a specific outcome, then some 30K genes over 10 organs would generate some 300K data points, which “reduce” down to a few alternative outcomes. The reduction of a 300K dimensional space to reduce down to 2-25 outcomes requires some dramatic pattern recognition capability.

The mathematical transformation from a 30K or 300K dimensional vector space down to the “critical” 3-5 outcome patterns is possible through the mathematics allowing identification of “eigen vectors”. The mathematical means to determine which markers are involved in the characteristic patterns are a first step. The goal it so identify what handful of “surrogate markers” can reproducibly and reliably correlate with the desired outcomes.

The data may be gene expression patterns, dynamic feature patterns, proteomic feature patterns, metabolomic measurement patterns, SNP evaluation patterns, or allele combination patterns. An advantage of using genetic based analyses is the lack of necessity to determine specifically where to evaluate the sample. Thus, genetic analysis will point to likely candidate markers whose number are sufficiently small to enormously reduce the complexity. With a much smaller universe of genes to investigate, coupled with large amounts of information on expression patterns, locations, disease contexts, systems biology, and the like, the difficulties of where, when, and how to look for the surrogate markers are greatly reduced.

The methodology to apply such to gene expression array analysis is described, e.g., in Najarian U.S. Pat. No. 6,996,476; Saffer, et al. U.S. Pat. No. 6,990,238; Parks and Moore U.S. Pat. No. 6,954,722; Lipshutz, et al. U.S. Pat. No. 6,953,663, and references contained therein, each of which is incorporated herein by reference. Outside of the context of gene expression, per se, e.g., in genotype, haplotype, or other high density characterization or based on other analysis means (e.g., proteomic or metabolomic analyses), earlier general mathematical methods analogous to those cited may be based on, e.g., Jackson (2003) A User's Guide to Principal Components Wiley-Interscience, ISBN 0471471348; Jolliffe (2002) Principal Component Analysis (2d Ed.) Springer, ISBN 0387954422; Diamantaras and Kung (1996) Principal Component Neural Networks: Theory and Applications Wiley-Interscience, ISBN 0471054364; Pearson (1901) “On Lines and Planes of Closest Fit to Systems of Points in Space” in The London, Edinburgh, and Dublin Philosophical Magazine and Journal of Science vol. 2:559-572, Sixth Series, July-December 1901; Hotelling (1933) “Analysis of a Complex of Statistical Variables into Principal Components” in The Journal of Educational Psychology 26:417-441 and 26:498-520; and Hotelling (1936) “Simplified Calculation of Principal Components” in Psychometrika 1:27-35.

Two basic categories of dimensionality reduction analyses are the supervised and unsupervised forms. See, e.g., Balakrishnama and Ganaphthiraju “Linear Discriminant Analysis: A Brief Tutorial” from the Department of Electrical and Computer Engineering, Institute for Signal and Information Processing, Mississippi State University. There are many algorithms for either method. Principal Component Analysis is an example of an unsupervised method, e.g., no assumptions are made regarding how many “patterns” are contained in the data. In contrast, the linear classifier (or linear discriminant) method is an example of supervised dimensionality reduction. For example, in a cancer compared to normal context, there are a priori categories (cancer, normal) and the effort is to identify a minimum set of genes or features that would discriminate between the two.

### VI. Selecting “Discriminating” Biomarker Subsets from Substantial Genome Pattern

The complexity of large dimensionality datasets from substantially genome-wide analysis overwhelms the data analysis capabilities of many studies. Thus, the conclusions from these studies often fail to recognize much of the information resulting from the study. In particular, upon proper analysis of these forms of experimentation, many additional lessons can be derived. In particular, the use and application of the methods of the present invention to existing data can lead to specific hypotheses which may be easily tested. Alternatively, the reduction in complexity of the system can allow more practical and modest testing procedures to focus more efficiently on the important and relevant markers.

In fact, often animal models fail to correlate to human disease. Thus, the identification of animal markers often fails to transfer to surrogate markers in humans. However, with more sophisticated data analysis and systems understanding, the relationship between different genes can be much better predicted. However, reducing the complexity and scale of screening or testing will be very important.

### A. Identifying Discriminating Marker Subsets

Screening for biomarkers in a complex system often fails to identify critical subsets of markers. In some cases, the parameter of analysis is not relevant. For example, RNA levels may not be relevant if the activity of the protein product is regulated by other than transcriptional means, e.g., post-translational phosphorylation regulation. Alternatively, measurable parameters, e.g., expression levels of specific tRNAs, often may be only marginally or irrelevant to a particular physiological state. In other cases, the manifestation of symptoms may not correspond to the initiation of the physiological cause. For instance, a metabolic deficiency may manifest itself in varied and different symptoms, or manifesting itself in different forms depending upon the individual.

Thus, a screening strategy which does not assume a cause casts a wider net for causes than a focused study based on unfounded assumptions. Genetic analysis can often identify markers which in complex systems otherwise would never have been presumed to have effects. However, once a genetic component is identified, that element may lead to identification of related elements, whether the interaction is physical or functional. Entities which modulate the effect of that initial element are candidates for further study, and alternative diagnostic markers, surrogate markers, or even targets for therapy.

Identification of relevant markers can focus efforts to screen for appropriate aspects of those markers. Screening a limited number of markers for the correct modality for assay, e.g., genetic allele pairings, complement of related overlapping functions, RNA transcription, protein expression, post-translational features, and the like can be readily performed. In particular, dynamic aspects of expression or functional activity can be monitored. See, e.g., Troyanskaya (2005) Brief Bioinform. 6:34-43; Abraham, et al. (2004) “High content screening applied to large-scale cell biology” Trends Biotechnol. 22:15-22; Sauer (2004) Curr. Opin. Biotechnol. 15:58-63; Daub, et al. (2003) Bioinformatics 19:2332-343; Nikitin, et al. (2003) Bioinformatics 19:2155-167; and similar publications.

An example of related analysis occurs in genetic studies. Selectable traits are correlated with particular identifiable genes or haplotypes. However, a converse problem can be conceived where the selectable trait has some correlation with the presence or absence of a haplotype, but the “penetrance” of the haplotype is less than striking. Penetrance is described, e.g., in Houlston and Peto (2004) Oncogene 23:6471-6476; Hirschhorn, et al. (2002) Genetics in Medicine 4:45-61; Human Genome Program, U.S. Department of Energy (2003) “Genomics and Its Impact on Science and Society: A 2003 Primer”. In such a situation, additional genetic features which affect penetrance, e.g., other “accompanying” haplotypes or genes, can be identified using the correct tools. See, e.g., Terwilliger and Weiss (1998) Curr. Op. Biotechn. 9:578-594. The penetrance might be correlated with “combinations” of genetic markers rather than with single markers. The present invention helps to suggest the forms of those accompanying markers.

### B. Selecting “Classifier” Subsets of Markers

Alternatively, where enormous amounts of data have been generated, one can select and scrutinize particular markers for those which provide the most advantageous features. Thus, features which make diagnostic evaluation more reliable will be preferred over less reliable features. These features include, among others, commercially relevant considerations on accuracy, reliability, minimum frequencies of false results, whether positive or negative, cost, speed, ease of evaluation, etc.

Regulatory concerns may also be important, e.g., speed and cost of regulatory approval, cost effectiveness, and others.

A combination of these two methodologies, using smaller homogeneous population for initial marker identification, with scrutiny of selected fractions, e.g., less than 80, 70, 60, 50, 40, or 30% of markers will provide efficiencies in the screening.

One first correlation often will be to identify genetic markers which correlate with a particular outcome, e.g., efficacy, safety, toxicity, ADR. The various common haplotype or allele combinations can be evaluated to see which combinations are more problematic than others, e.g., whether various specific allele combinations correlate with greater or lesser response. With the identification of the markers, pathway analysis will point to other markers which are likely to similarly affect the main toxicity mechanism pathways. Those alternative markers will often also be tested to determine whether those also correlate with various outcomes, and alternatives may be screened for favorable diagnostic or clinical characteristics.

Attractive features for preferred biomarkers include, e.g., (1) stable background across individuals, time, and conditions; (2) high signal with low noise; (3) easy sample accessibility (preferably minimally invasive, perhaps blood, urine, tear, or surface sampling); (4) accurate, simple, rapid platform for analysis; (5) high statistical accuracy and correlation with outcome; (6) high reproducibility in measurements; (7) inexpensive and simple analytical methods.

### VII: Pathway Identification to Critical Points

Knowledge databases linking data within a species or across species will be useful. There are many such databases, e.g., products similar to those provided by Ingenuity, Entelos, BioVista, Jubilant BioSystems, ARIADNE Genomics, and the like. Similar offerings should be available from the NCBI website and others. See, e.g., http://www.genomicglossaries.com/content/lifesciences_databasesdirectory.asp, which refer to various specific databases, e.g., Metabolic Pathways of Biochemistry (www.gwu.edu), Kyoto Encyclopedia of Genes and Genomes (KEGG, www.genome.jp/kegg), ExPASys (www.expasy.ch), Enzymes and Metabolic Pathways Project (www.empproject.com), metacyc encyclopedia of metabolic pathways (metacyc.org), Alliance for Cellular Signaling (AfCS, www.cellularsignaling.org), biopathways consortium (www.biopathways.org), and similar sites.

From haplotypes which are correlated with defined pathways and networks, other members of those networks will be identified and their relevance to the pathway can be similarly inferred. From here, different strategies will be applied, depending partly on the difficulty of phenotype readout. Some phenotypes may be directly observed in experimental systems, either in vivo, or in vitro or subsystems. Other phenotypes will be analyzed to determine either surrogate markers, or similar signatures of important physiological or other precursor indicators of phenotype.

These further analyses may take many forms, e.g., directed to metabolic evaluations (metabolomic endpoints), structural endpoints (determining whether structural components of the respective subcellular or organ structure have effects), regulatory effects (e.g., using RNAi, antisense, genetic targeting, or other means to affect gene expression levels), gene mutational analyses, multiple endpoint analyses over time or other parameters, solvent effects, and others. These will provide a simplistic form of determining what effects are direct, indirect, or highly correlative with phenotype. Many different combinations may be used, and may provide alternative parameters with multidimensional readouts. This will generally provide a more reliable and stable diagnostic approach.

Perturbation analyses can be performed to identify particular nodes in the networks which are especially sensitive or stable to perturbation. See, e.g., Ramanathan, et al. (2005) “Perturbational Profiling of a Cell Line Model of Tumorigenesis by using Metabolic Measurements” Proc. Nat'l Acad. Sci. USA 102:5992-5997. The perturbation analyses described herein can be applied to signatures incorporating different parameters of measurement.

### VIII. Correlation to Identify Biomarkers/Signatures in Organ or System Response

Correlation of biomarkers to phenotype occurs in various contexts. In particular, surrogate biomarkers are used as diagnostic of a phenotype, and may be useful, e.g., in in vitro systems (which correlate with the in vivo whole organism context), or in model systems to predict phenotype in a whole animal. See, e.g., McKim, et al. (2005) “A Biochemical Approach to In Vitro Toxicity Testing” Pharmaceutical Discovery, January 2005. An approach is described which successfully bridged the gap between cells and whole-animal effects by focusing on key functional endpoints that are not organ- or species-specific. The capability has been validated in animal studies. Certain biomarkers or endpoints may be used to predict cross-species results.

In the present invention, curve fitting of signatures back to phenotype of human response to administered drug (adverse drug response) is based on human responses. Experimental systems can be evaluated for surrogate markers which fit the human response outcome. Thus, cross species issues are minimized for whether results in a non-human species are relevant to humans.

Test systems may include in vitro and in vivo (animal models). These often will incorporate genetic features which increase susceptibility to, or simulate, a disease or physiological context. The systems may be used to test or screen chemical or biologic compounds for response with appropriate classifier markers, or for status of a toxicity pathway. Where the classifier markers are highly correlative with human response, the test system has high reliability. Thus, the test system may use a different specie background with selective components of human markers, or human with specific allelic combinations correlative with individuals in the human population. Moreover, the diversity of responses may be evaluated in the test system.

Conversely, test systems may have poor correlation with outcome in the intact organism. In such circumstances, the curve fitting to find features or appropriate parameters for accurate prediction should relate to outcome in the intact physiological context. Thus, using primate, and preferably human subjects, will allow determination of the genetic markers which promote penetrance.

### IX. Extension of Identified Biomarkers to Related Alternatives

Upon initial identification of correlating biomarkers, different signatures can be derived therefrom. For example, once identified in one species, it is likely that counterpart structural or functional entities can be found in closely related species. Systems biology and/or genomic data can be used to identify those counterparts, or other experimental methods may be applied to find counterparts in different species.

It will also often be true that functions closely related, including upstream or downstream functionalities, will also have statistical correlation with phenotype, and may move away from or towards critical checkpoints. Thus, it will be useful, once biomarkers are identified, to specifically check whether functionally adjacent biomarkers will show similar or greater correlation.

In addition, by looking at haplotype or allelic pair statistics, it should be possible to identify what pairings may attenuate or otherwise affect phenotype. Where multiple entities (e.g., different allelic variants) have similar or overlapping reaction specificities (e.g., for a substrate), it will be useful to see whether the phenotype also is dependent upon co-expression with related entities. In the situation of multisubunit complexes, the spectrum of alternative combinations of the respective components should be important, and explicitly investigated. For standard haplotype or allele combinations, some pairings may exhibit greater or lesser penetrance, and, e.g., phenotype should correlate more directly with combinations of paired alleles than with either allele alone.

When important signatures are identified, the dynamics of interactions or of metabolic conversions should be evaluated (e.g., metabolic fingerprints). The timing signature of the various biomarkers or metabolites should be carefully evaluated to identify temporal features which can be important in appearance of phenotype. Recognizing such features may also allow for dynamic monitoring of phenotype emergence.

The signatures may be in the intact organism, or may be model systems, e.g., in situ, in vitro, or subsystems. Once the appropriate network is identified, components thereof are identified as features to be evaluated for signatures. These features (specific species counterparts or variants) may also be targeted, e.g., for perturbation analysis, by substituting or incorporating various allelic forms into cells or systems as described, by transformation, deletion, etc. Allelic variants may take many forms, e.g., incorporating specific changes characteristic of natural variants, regulatory changes, enzymatic activity modulators, and the like. Such cell lines have been generated directed to cytochrome P450 pathway alleles or variants, e.g., incorporated into hepatocytes. See, e.g., Miners, et al. (2004) Ann. Rev. Pharmacol. Toxicol. 44:1-25; Rushmore and Kong (2002) Curr. Drug Metab. 3:481-490; Arora and Iversen (2001) Curr. Opin. Mol. Ther. 3:249-257; and Goldstein and de Morais (1994) Pharmacogenetics 4:285-299.

Often most relevant systems may be subsystems or cell lines placed in the context of a relevant disease model. The cell lines or systems may be genetically modified, e.g., incorporating different genes, differently regulated genes, combinations of human or relevant allelic forms, and the like. Human or other stem cells or their derivatives, e.g., differentiated organ systems may be useful. The systems may include physiologically or developmentally affected cells, cell lines, organs, tissues, or the like. And the systems may incorporate technologies relating to “laboratory on a chip” microfluidics or highly parallel or robotic systems. Individual chips, cells, lines, or systems may represent different individuals whose variability may be analogous to populational variability. Series of cells or systems may incorporate ranges of expression levels, attenuating, suppressing, competing, selective agents, and the like to evaluate ranges of responses to such perturbations.

### X. Experimental Models Using Identified Target Analytes

### A. Predictive Models

A predictive model may be built that combines several of these “patterns”, and can be used experimentally to predict from the preclinical model the phenotype which will result upon actual administration to an intact animal. This is a “curve fitting” exercise, which correlates the pattern features to the effect endpoints, which can be a human population (e.g., in an ADR correlation). This can be used to validate a model, and help establish that the model does, using the identified signatures, correlate with human outcomes.

### B. Testing Historic Compounds

Testing of compounds which have been used historically will typically be simple in terms of in vitro or small animal testing. Similarly, where a history of use in humans has existed and records or tracking of those persons has been performed, e.g., in an ADR report, the human outcomes are known. These may be in ADR databases, or archived in clinical trials. Thus, defined populations with such data can be extremely useful to evaluate genetically, as described above, and by the methods described.

### C. Testing or Screening NCEs

New clinical candidates (NCEs) or new compounds can be tested in the experimental systems described to get accurate, low cost readouts on important clinical outcomes. In early stages of program development, these experimental systems may be used to prioritize candidates, predict preclinical or clinical outcomes, or determine when medicinal chemistry efforts have accumulated sufficient candidates of acceptable properties. The systems described herein may allow better informed decision making for early stage research efforts. Certainly advances made in early decision making will minimize wastes from later expensive testing which confirms unsuitability for clinical development.

### XI. Curve Fitting from Experimental Analytes to Outcomes

Various methods have been used to predict whole animal toxicity effects using in vitro experimental systems. See, e.g., McKim, et al. (January 2005) Pharmaceutical Discovery pp 30-36; (see www.Ceetox.com).

In a rat system, in vitro methods and measured parameters after exposure to various selected compounds have been correlated with outcomes from in vivo animal studies. These parameters have then been used to predict the outcome of administration of similar compounds to whole animal exposure.

In cellular in vitro assays, typically selected cell types are seeded into microtiter plates and allowed to grow and equilibrate for a period of time. Certain cells may be selected for increased or decreased function of relevant biomarkers, e.g., transmembrane pumps, cytochromes, important metabolizing enzymes, etc. Various mutants may be evaluated, including cells lacking or overexpressing particular functions, transformed with multiple copies of specific variants or alleles, or important combinations of functions, e.g., increased intake with decreased excretion.

The period for equilibration typically ranges from 12-48 hrs, depending, e.g., upon cell type, density, and other factors. Thereafter, the test compounds are applied to the cells across broad concentration ranges to near solubility limits, typically 2-4 logs, and generally across 1-300 microM. Negative and positive controls are included.

Following such exposure, the cells and supernatants are evaluated, typically for general cellular processes. These typically include some or all of membrane integrity, mitochondrial function, cell proliferation, glutathione biochemistry, membrane lipid peroxidation, apoptosis, DNA integrity, transporter function, adsorption or excretion activity, and general biological functions. Further pharmacology evaluation will typically include ADME, e.g., solubility measurement in the delivery medium, Pgp interaction, comparison of toxicity relative to related chemicals (structural or functional), subcellular targets of toxicity, and some component of structure-toxicity relationships. Other effects to evaluate include enzyme induction, metabolic activation, endocrine disruption, lipidosis, etc.

### XII. Experimental Model Predictions to Human Effects

Various diagnostic markers derived from the methods described herein will be useful as surrogate markers. These can applied in specific model systems as signatures of phenotypic outcomes, e.g., phenotypes. When the signatures are accepted, e.g., from biological reasoning or by experimental demonstration, the phenotypes may be inferred from the appropriate signature. The models will then be useful as early screening tools for evaluating phenotype.

### A. Efficacy in Individuals

The predictions may be directed to subsetting potential patients into groups with predetermined likelihood of efficacious therapeutic treatment. Thus, e.g., some set of diagnostic features might subset target individuals into defined groups where there is high, questionable, or low likelihood of specific outcome (phenotype). While this may reflect better definition of clinical problem (e.g., distinguishing between various “forms” of a syndrome or of a “group” of similarly defined symptomatic conditions), it may provide for subsetting into groups likely to respond to a proposed therapeutic regimen.

### B. Specific Drug Effects

Similarly, the predictions may be directed to subsetting potential patients into groups with predetermined likelihood of a particular effect of therapeutic treatment. Thus, e.g., some diagnostic signature might subset target individuals into defined groups where there is high, questionable, or low likelihood of specific outcome, particularly a positive or negative result, be it direct or indirect. This will particularly be valuable in the context of recognizing genetic or other predispositions to adverse side effects or intolerance to a particular proposed treatment. These factors may even be incorporated into the label of particular therapeutic entities or procedures.

All references cited herein are incorporated herein by reference to the same extent as if each individual publication or patent application was specifically and individually indicated to be incorporated by reference in its entirety for all purposes.

Having now fully described this invention, it will be appreciated by those skilled in the art that the same can be performed within a wide range of equivalent parameters, amounts, and conditions without departing from the spirit and scope of the invention and without undue experimentation. While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

The specific embodiments described herein are offered by way of example, and the invention is to be limited only by the terms of the appended claims, along with the full scope of equivalents to which such claims are entitled.

1. A method for inferring clinical outcome to the administration of a medicament to a human or animal subject from a nucleic acid sample of the subject, the method comprising identifying, in the nucleic acid sample, at least one diploid pair of haplotype alleles indicative of said clinical outcome, wherein the diploid pair of haplotype alleles is identified by (a) analyzing diploid pairs of haplotype alleles of individuals selected from a homogeneous population and (b) correlating clinical outcomes from medical data of said population to the analysis in step (a).

2. The method of claim 1 further comprising identifying in said nucleic acid sample one or more additional haplotypes, wherein expression of said one or more haplotypes and said at least one diploid pair of haplotype alleles indicative of said clinical outcome define an expression profile and wherein identifying said expression profile increases the statistical accuracy of inferring said clinical outcome compared to identifying said at least one diploid pair of haplotype alleles alone.

3. The method of claim 2 wherein said expression profile comprises changes in the expression of said haplotypes in the same individual over a predetermined period of time.

4. The method of claim 2 wherein said one or more additional haplotypes (1) are identified in an interaction database to interact physically with said at least one diploid pair of haplotype alleles indicative of said clinical outcome; (2) are commonly referred to in a scientific literature reference with said at least one diploid pair of haplotype alleles indicative of said clinical outcome; (3) are expressed with profiles that match the expression profile of said at least one diploid pair of haplotype alleles indicative of said clinical outcome in similar tissues; (4) are co-localized in expression analyses in similar tissues as said at least one diploid pair of haplotype alleles indicative of said clinical outcome; or (5) are located physically on a chromosome close to said at least one diploid pair of haplotype alleles indicative of said clinical outcome.

5. The method of claim 2 wherein said one or more additional haplotypes play a role in a biological pathway upstream or downstream from said diploid pair of haplotype alleles indicative of said clinical outcome.

6. The method of claim 1 wherein step (a) comprises identifying a gene from a non-human species and mapping said gene to a human counterpart which is a member of a diploid pair of haplotype alleles analyzed in step (a).

7. A computer-implemented method of identifying diploid pairs of haplotype alleles useful for predicting clinical outcome of an individual to therapeutic treatment, comprising: (a) obtaining gene expression profile data from a homogeneous population of individuals having received the treatment with a recorded result from the treatment; (b) analyzing diploid pairs of haplotype alleles of individuals selected from said homogeneous population (c) correlating clinical outcomes from medical data of said population to identify diploid pairs of haplotype alleles which correlate with said clinical outcome, and (d) viewing the results of step (c) to facilitate the identification of said diploid pairs of haplotype alleles indicative of said clinical outcome.

8. A method for generating a database of biomarkers useful for predicting a clinical outcome of an individual to therapeutic treatment, comprising: (a) collecting a homogeneous population of individuals having received the treatment with a recorded result from the treatment; (b) analyzing diploid pairs of haplotype alleles of individuals selected from said homogeneous population (c) correlating clinical outcomes from medical data of said population to identify diploid pairs of haplotype alleles which correlate with said clinical outcome, and (d) recording diploid pairs of haplotype alleles from step (c) in a computer readable database.

9. A business method for commercializing a database of biomarkers useful for predicting a clinical outcome of an individual to therapeutic treatment, comprising: (a) collecting a homogeneous population of individuals having received the treatment with a recorded result from the treatment; (b) analyzing diploid pairs of haplotype alleles of individuals selected from said homogeneous population (c) correlating clinical outcomes from medical data of said population to identify diploid pairs of haplotype alleles which correlate with said clinical outcome, (d) recording diploid pairs of haplotype alleles from step (c) in a computer readable database; and (e) marketing said database.

10. The method of claim 1 wherein analyzing diploid pairs of haplotype alleles of individuals selected from a homogeneous population comprises analyzing diploid data based on heterozygote models.

11. The method of claim 1 wherein analyzing diploid pairs of haplotype alleles of individuals selected from a homogeneous population comprises separating the alleles in diploid pairs and comparing results of the analysis of the separated pairs to the analysis of diploid pairs.

12. The method of claim 1 wherein analyzing diploid pairs of haplotype alleles of individuals selected from a homogeneous population comprises RNA analysis, postranslational analysis, or immunoassay based analysis.

13. A method for detecting the state of a toxicity pathway in a primate, said method comprising evaluating the form or function of one or more discriminatory biomarker selected from Table 2A subset 1, subset 2 or subset 3; Table 3A subset 1, subset 2 or subset 3 Table 4, subset 1 or subset 2; Table 5A subset 1, subset 2 or subset 3; or Table 6A subset 1, subset 2 or subset 3.

14. The method of claim 13, wherein: a) said toxicity pathway is expressed in liver; cardiovascular tissue, neural tissue, kidney, PBMC (peripheral blood mononuclear cell), skin, and/or lung b) said form of evaluating is a protein evaluation, including an immunoassay, modification, quantitation, mass spectroscopy, NMR, imaging, or characteristic temporal pattern determination; c) said evaluating is from a blood, hair, skin, saliva, oral mucosa, tears, mucus, or other accessible body fluid sample; d) said discriminatory biomarker is from a subset 1 of Tables 3A, 5A or 6A; e) said discriminatory biomarker is from a subset 2 or 3 of Tables 3A, 5A or 6A; and/or f) said discriminatory biomarker is derived from two or more datasets from Tables 3A, 4A, 5A and 6A.

15. A toxicology pathway evaluation kit comprising instructions for use and a diagnostic means directed to an evaluating discriminatory biomarker for use in a method according to claim 14.

16. A biological system to test candidate compounds for inducing a liver or other toxicity pathway, wherein said system comprises multiple biological samples exhibiting common combinations of alleles corresponding to biomarkers for use in the method of claim 14, thereby representing response among individuals represented by said combinations.

17. A method for detecting the state of a toxicity pathway in a primate, said method comprising evaluating the form or function of at least 3 or more discriminatory biomarkers selected from the subsets 3of Tables 3A, 5A and 6A.

18. A method of identifying additional relevant genes as candidate test targets for toxicity pathway evaluation, comprising providing a first list of candidate targets and identifying a second list of additional candidate targets: 1) which in an interaction database have been reported to interact physically with said targets of list 1 and/or list 2, including a physical interaction or 2-hybrid physical interaction; 2) which have been commonly referred to in a reference with a target of list 1 and/or list 2, said reference being in the abstract of a paper contained in a literature database; 3) whose gene expression profiles match the expression profiles of those members of list 1 and/or list 2 in similar tissues; 4) which have been co-localized in expression analyses in similar tissues; or 5) which are closely located physically on a chromosome.

19. The method of claim 18, wherein said toxicity is evaluated in a mammal, and said method comprising evaluating a plurality of features at multiple time points with use of an extended primate group whose members are selected based on their propensity to be phenotypically homogeneous, as indicated by medical record or other selection criteria, or are genetically homogeneous.

20. The method of claim 18, wherein list 1 and/or list 2 contain 1, 2, 3, 4, 5, 6, 7, 8, 9, 10, 15, 20, 25, 50, 75, 100, 200, 500, 1000 or more genes.

21. A method according to claim 1, wherein said clinical outcome comprises an adverse reaction, a favorable therapeutic result, an unfavorable therapeutic result, an efficacy result or an absence of therapeutic response to the administration of said medicament or therapeutic treatment.

22. A method according to claim 7, wherein said clinical outcome comprises an adverse reaction, a favorable therapeutic result, an unfavorable therapeutic result, an efficacy result or an absence of therapeutic response to the administration of said medicament or therapeutic treatment.

23. A method according to claim 8, wherein said clinical outcome comprises an adverse reaction, a favorable therapeutic result, an unfavorable therapeutic result, an efficacy result or an absence of therapeutic response to the administration of said medicament or therapeutic treatment.

24. A method according to claim 9, wherein said clinical outcome comprises an adverse reaction, a favorable therapeutic result, an unfavorable therapeutic result, an efficacy result or an absence of therapeutic response to the administration of said medicament or therapeutic treatment.
